(19) United States
(12) Patent Application Publication
(10) Pub. No.: US 2024/0083886 A1
(43) Pub. Date: Mar. 14, 2024

(54) HETEROCYCLIC GLP-1 AGONISTS

(71) Applicant: Gasherbrum Bio, Inc., South San Fracisco, CA (US)

(72) Inventors: Qinghua MENG, Shanghai (CN); Weiqiang XING, Shanghai (CN); Xichen LIN, Shanghai (CN); Andrew JENNINGS, San Diego, CA (US)

(21) Appl. No.: 18/248,858

(22) PCT Filed: Oct. 13, 2021
(86) PCT No.: PCT/CN2021/123515

§ 371 Date: Apr. 12, 2023

(30) Foreign Application Priority Data
Oct. 13, 2020 (CN) 120605
Sep. 14, 2021 (CN) 118149

(51) Int. Cl. C07D 405/14; C07D 471/04; C07D 413/14; C07D 487/04; C07D 417/14

(52) U.S. Cl. CPC C07D 405/14; C07D 471/04; C07D 413/14; C07D 487/04; C07D 417/14

(57) ABSTRACT

Relates to GLP-1 agonists of Formula (I): including pharmaceutically acceptable salts and solvates thereof, and pharmaceutical compositions including the same.

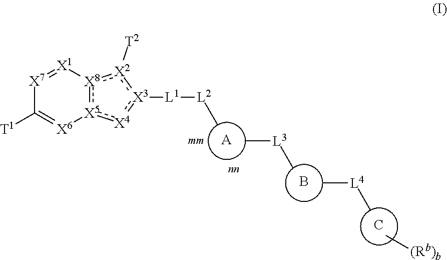

### CROSS REFERENCE TO RELATED APPLICATIONS

This application claims the benefit of International Patent Application Number PCT/CN2020/120605, filed on Oct. 13, 2020; and International Application Number PCT/CN2021/118149, filed on Sep. 14, 2021 each of which is incorporated herein by reference in its entirety.

### TECHNICAL FIELD

This disclosure relates to GLP-1 agonists, pharmaceutical compositions, and methods of use thereof.

### BACKGROUND

Incretin metabolic hormones, including glucagon-like peptide-1 (GLP-1) and glucose-dependent insulinotropic polypeptide (GIP), are important in the regulation of glucose homeostasis. Medicaments targeting this family of intestinal peptides, such as GLP-1 agonists, have been shown to suppress glucagon production, decrease gastric motility, and increase satiety.

Diabetes mellitus refers to a group of metabolic disorders characterized by persistent hyperglycemia. The most common form, type 2 diabetes mellitus (T2DM) is an acquired condition that accounts for more than 90% of diabetes cases. Typical onset occurs in obese or otherwise sedentary adults and begins with insulin resistance. Though lifestyle changes can be useful in management of this disorder, patients with T2DM may be required to take antidiabetic medications, including dipeptidyl peptidase-4 inhibitors, SGLT2 inhibitors, and sulfonylureas, among others.

In healthy individuals, the incretin hormones glucose-dependent insulinotropic polypeptide (GIP) and glucagon-like peptide 1 (GLP-1) provide tandem modulation of insulin secretory response to glucose ingestion. While this incretin effect is significantly diminished (if at all present) in cases of T2DM, GLP-1 retains insulinotropic properties, even as endocrine pancreatic response to GIP is effectively halted. As such, incretin mimetics and other GLP-1-based therapies can help stimulate insulin production in T2DM patients.

### SUMMARY

The present application describes heterocyclic GLP-1 agonists, as well as pharmaceutical compositions comprising the compounds disclosed herein. Also provided are methods for treating GLP-1-associated diseases, disorders, and conditions.

Accordingly, provided herein are compounds of Formula I:

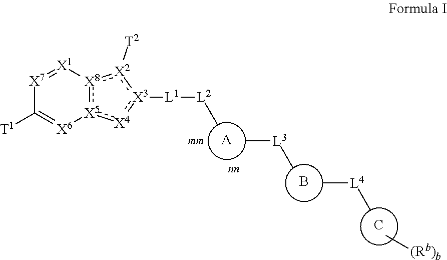

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

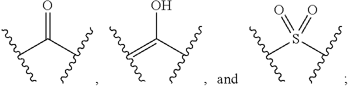

5- to 10-membered heterocycloalkyl, wherein the heterocycloalkyl comprises an endocyclic group selected from the group consisting of:

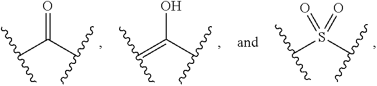

wherein the heterocycloalkyl is optionally substituted with 1-4 R^{v}; and
   (C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

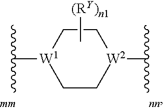

wherein n1 is 0, 1, or 2; W¹ is CR^{Y1} or N; and W² is CR^{Y2} or N;

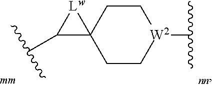

wherein W² is CR^{Y2} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};
wherein mm represents the point of attachment to L², and nn represents the point of attachment to L³
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is selected from the group consisting of: —O—; —S—; —C(R^{a}R^{a})—; —N(H)—; —N(C₁₋₃ alkyl)-; —C(═O)—; and —S(O)₁₋₂—,
each occurrence of R^{a} is independently selected from the group consisting of: hydrogen, halogen, CN, —OH, (C₁-C₆)alkyl, (C₁-C₃)haloalkyl, (C₃-C₈)cycloalkyl, (C₁-C₃)alkoxy, and (C₁-C₃)haloalkoxy; or
a pair of R^{a} taken together with the carbon atom to which each is attached forms a (C₃-C₈)cycloalkyl ring; or
when Ring A is

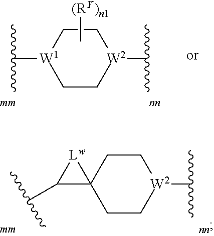

W² is CR^{Y2}; and L³ is —C(R^{a}R^{a})—, one R^{a} combines with R^{Y2} to form a double bond between W² and L³, wherein the remaining R^{a} is selected from the group consisting of: hydrogen, halogen, CN, (C₁-C₆)alkyl, (C₁-C₃)haloalkyl, and (C₃-C₈)cycloalkyl;
Ring B is selected from the group consisting of: (B-I), (B-II), (B-III), (B-IV), and (B-V):

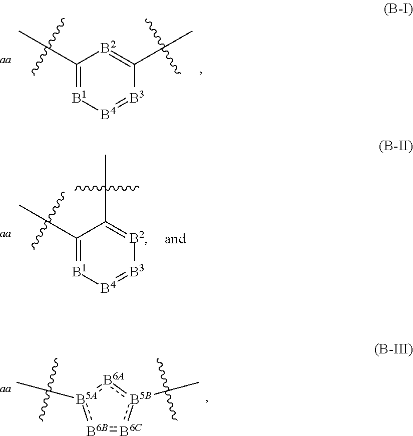

wherein aa represents the point of attachment to L³.
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

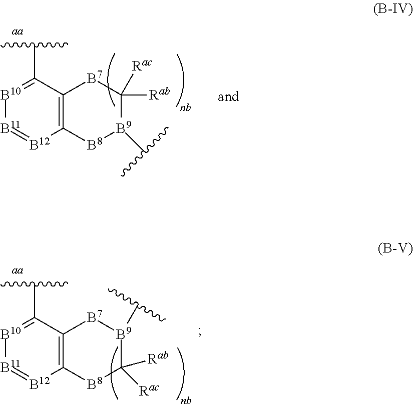

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Further provided herein are compounds of Formula I:

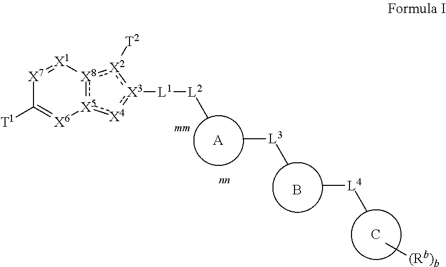

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

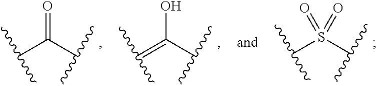

5- to 10-membered heterocycloalkyl, wherein the heterocycloalkyl comprises an endocyclic group selected from the group consisting of:

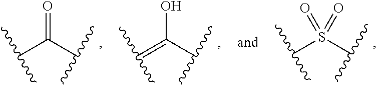

wherein the heterocycloalkyl is optionally substituted with 1-4 R^{v}; and
   (C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
or when q is 2 or 3, a pair of R^{x} on adjacent carbon atoms, taken together, form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, —O(C₁-C₆)alkylene-O(C₁-C₆)alkyl, phenyl, —S(O)₀₋₂—C₁-C₆ alkyl, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

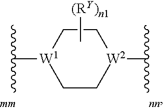

wherein n1 is 0, 1, or 2; W¹ is CR^{Y1} or N; and W² is CR^{Y2} or N;

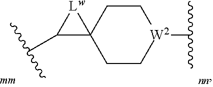

wherein W² is CR^{Y2} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};
wherein mm represents the point of attachment to L², and nn represents the point of attachment to L³
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is selected from the group consisting of: —O—; —S—; —C(R^{a}R^{a})—; —N(H)—; —N(C₁₋₃ alkyl)-; —C(═O)—; and —S(O)₁₋₂—,
each occurrence of R^{a} is independently selected from the group consisting of: hydrogen, halogen, CN, —OH, (C₁-C₆)alkyl, (C₁-C₃)haloalkyl, (C₃-C₈)cycloalkyl, (C₁-C₃)alkoxy, and (C₁-C₃)haloalkoxy; or
a pair of R^{a} taken together with the carbon atom to which each is attached forms a (C₃-C₈)cycloalkyl ring; or
when Ring A is

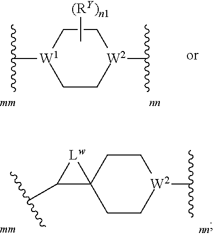

W² is CR^{Y2}; and L³ is —C(R^{a}R^{a})—, one R^{a} combines with R^{Y2} to form a double bond between W² and L³, wherein the remaining R^{a} is selected from the group consisting of: hydrogen, halogen, CN, (C₁-C₆)alkyl, (C₁-C₃)haloalkyl, and (C₃-C₈)cycloalkyl;
Ring B is selected from the group consisting of: (B-I), (B-II), (B-III), (B-IV), and (B-V):

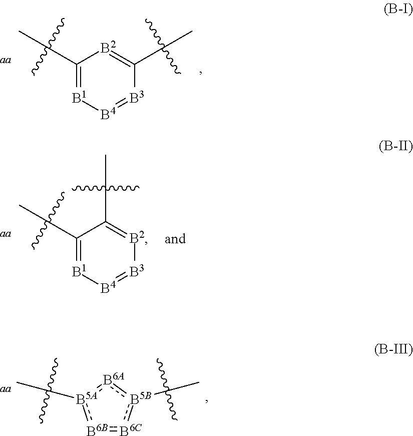

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

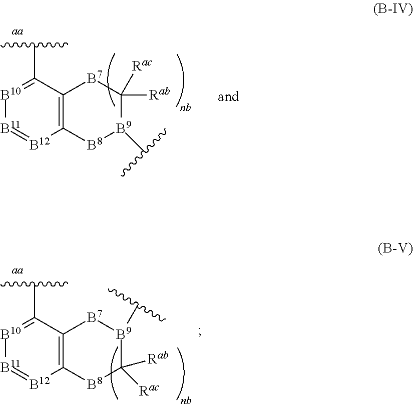

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Further provided herein are compounds of Formula I:

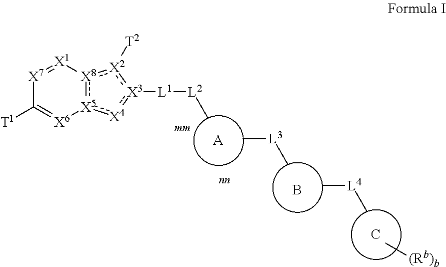

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

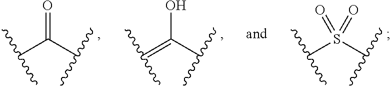

5- to 10-membered heterocycloalkyl, wherein the heterocycloalkyl comprises an endocyclic group selected from the group consisting of:

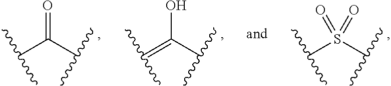

wherein the heterocycloalkyl is optionally substituted with 1-4 R^{v}; and
   (C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
or when q is 2 or 3, a pair of R^{x} on adjacent carbon atoms, taken together, form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, —O(C₁-C₆)alkylene-O(C₁-C₆)alkyl, phenyl, —S(O)₀₋₂—C₁-C₆ alkyl, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

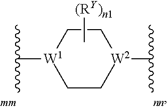

wherein n1 is 0, 1, or 2; W¹ is CR^{Y1} or N; and W² is CR^{Y2} or N;

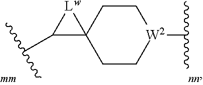

wherein W² is CR^{Y2} or N, L^{w} is (C₁-C₃)alkylene;
phenylene optionally substituted with 1-4 R^{Y};
5- to 6-membered heteroarylene optionally substituted with 1-3 R^{Y};
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y};

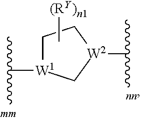

wherein n1 is 0, or 2; W¹ is CR^{Y1} or N; and W² is CR^{Y2} or N; wherein mm represents the point of attachment to L², and nn represents the point of attachment to L³
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is selected from the group consisting of: —O—; —S—; —C(R^{a}R^{a})—; —N(H)—; —N(C₁₋₃ alkyl)-; —C(═O)—; and —S(O)₁₋₂—,
each occurrence of R^{a} is independently selected from the group consisting of: hydrogen, halogen, CN, —OH, (C₁-C₆)alkyl, (C₁-C₃)haloalkyl, (C₃-C₈)cycloalkyl, (C₁-C₃)alkoxy, and (C₁-C₃)haloalkoxy; or
a pair of R^{a} taken together with the carbon atom to which each is attached forms a (C₃-C₈)cycloalkyl ring; or
when Ring A is

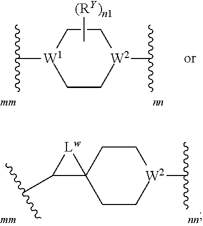

W² is CR^{Y2}; and L³ is —C(R^{a}R^{a})—, one R^{a} combines with R^{Y2} to form a double bond between W² and L³, wherein the remaining R^{a} is selected from the group consisting of: hydrogen, halogen, CN, (C₁-C₆)alkyl, (C₁-C₃)haloalkyl, and (C₃-C₈)cycloalkyl;
Ring B is selected from the group consisting of: (B-I), (B-II), (B-III), (B-IV), and (B-V):

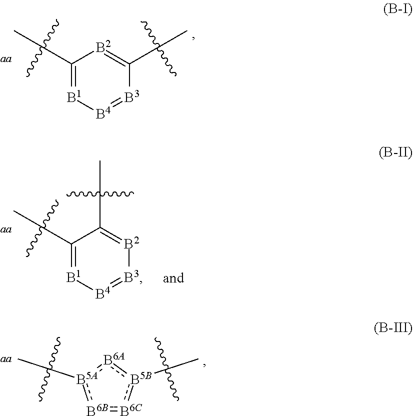

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

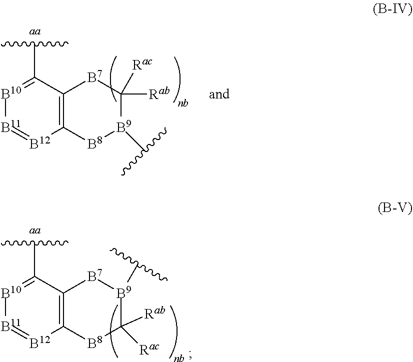

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Also provided herein are pharmaceutical compositions comprising a compound of Formula I, or a pharmaceutically acceptable salt or solvate thereof, and a pharmaceutically acceptable excipient.

Also provided herein are methods for treating type 2 diabetes mellitus in a patient in need thereof, the methods comprising administering to the patient a therapeutically effective amount of a compound of Formula I, or a pharmaceutically acceptable salt or solvate thereof, or a pharmaceutical composition thereof.

Also provided herein are methods for treating type 2 diabetes mellitus in a patient, the methods comprising administering to a patient identified or diagnosed as having type 2 diabetes mellitus a therapeutically effective amount of a compound of Formula I, or a pharmaceutically acceptable salt or solvate thereof, or a pharmaceutical composition thereof.

Also provided herein are methods for treating diabetes mellitus in a patient, the methods comprising determining that the patient has type 2 diabetes mellitus; and administering to the patient a therapeutically effective amount of a compound of Formula I, or a pharmaceutically acceptable salt or solvate thereof, or a pharmaceutical composition thereof. In some embodiments, the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof. In some embodiments, the level of HbA1c is greater than or about 6.5%. In some embodiments, the level of fasting plasma glucose is greater than or about 126 mg/dL. In some embodiments, the level of non-fasting plasma glucose is greater than or about 200 mg/dL.

In some embodiments, the methods further comprise obtaining a sample from the patient. In some embodiments, the sample is a body fluid sample. In some embodiments, the patient is about 40 to about 70 years old and is overweight or obese. In some embodiments, the patient has a body mass index (BMI) greater than or about 22 kg/m². In some embodiments, the patient has a BMI greater than or about 30 kg/m². In some embodiments, the methods for the treatment of type 2 diabetes mellitus comprise a reduction in fasting plasma glucose levels. In some embodiments, the fasting plasma glucose levels are reduced to about or below 100 mg/dL.

In some embodiments, the methods for the treatment of type 2 diabetes mellitus comprise a reduction in HbA1c levels. In some embodiments, the HbA1c levels are reduced to about or below 5.7%.

In some embodiments, the methods for the treatment of type 2 diabetes mellitus comprise a reduction in glucagon levels.

In some embodiments, the methods for the treatment of type 2 diabetes mellitus comprise an increase in insulin levels.

In some embodiments, the methods for the treatment of type 2 diabetes mellitus comprise a decrease in BMI. In some embodiments, the BMI is decreased to about or below 25 kg/m².

In some embodiments, the compound of Formula I, or a pharmaceutically acceptable salt or solvate thereof, or a pharmaceutical composition thereof, is administered orally.

In some embodiments, the methods of treatment for type 2 diabetes mellitus further comprise administering an additional therapy or therapeutic agent to the patient. In some embodiments, the additional therapy or therapeutic agent is selected from the group consisting of an antidiabetic agent, an anti-obesity agent, a GLP-1 receptor agonist, an agent to treat non-alcoholic steatohepatitis (NASH), anti-emetic agent, gastric electrical stimulation, dietary monitoring, physical activity, or any combinations thereof. In some embodiments, the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof. In some embodiments, the biguanide is metformin. In some embodiments, the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof. In some embodiments, the GLP-1 receptor agonist is selected from the group consisting of liraglutide, exenatide, dulaglutide, albiglutide, taspoglutide, lixisenatide, semaglutide, or any combinations thereof. In some embodiments, the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof. In some embodiments, the compound of Formula I, or a pharmaceutically acceptable salt or solvate thereof, or a pharmaceutical composition thereof, and the additional therapeutic agent are administered as separate dosages sequentially in any order.

Also provided herein are methods for modulating insulin levels in a patient in need of such modulating, the method comprising administering to the patient an effective amount of a compound of Formula I, or a pharmaceutically acceptable salt or solvate thereof, or a pharmaceutical composition thereof. In some embodiments, the modulation results in an increase of insulin levels.

Also provided herein are methods for modulating glucose levels in a patient in need of such modulating, the method comprising administering to the patient an effective amount of a compound of Formula I, or a pharmaceutically acceptable salt or solvate thereof, or a pharmaceutical composition thereof. In some embodiments, the modulation results in a decrease of glucose levels.

Also provided herein are methods for treating a GLP-1 associated disease, disorder, or condition, the method comprising administering to a patient in need thereof an effective amount of a compound of Formula I, or a pharmaceutically acceptable salt or solvate thereof, or a pharmaceutical composition thereof. In some embodiments, the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof. In some embodiments, the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof. In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

Other features and advantages of the invention will be apparent from the following detailed description and figures, and from the claims.

### DETAILED DESCRIPTION

Provided herein are heterocyclic GLP-1 agonists for use in the management of T2DM and other conditions where activation of GLP-1 activity is useful.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

As used herein, the term “halo” or “halogen” means —F (sometimes referred to herein as “fluoro” or “fluoros”), —Cl (sometimes referred to herein as “chloro” or “chloros”), —Br (sometimes referred to herein as “bromo” or “bromos”), and —I (sometimes referred to herein as “iodo” or “iodos”).

As used herein, the term “alkyl” refers to saturated linear or branched-chain monovalent hydrocarbon radicals, containing the indicated number of carbon atoms. For example, “(C₁-C₆)alkyl” refers to saturated linear or branched-chain monovalent hydrocarbon radicals of one to six carbon atoms. Non-limiting examples of alkyl include methyl, ethyl, 1-propyl, isopropyl, 1-butyl, isobutyl, sec-butyl, tert-butyl, 2-methyl-2-propyl, pentyl, neopentyl, and hexyl.

As used herein, the term “alkylene” refers to a divalent alkyl containing the indicated number of carbon atoms. For example, “(C₁-C₃)alkylene” refers to a divalent alkyl having one to three carbon atoms (e.g., —CH₂—, —CH(CH₃)—, —CH₂CH₂—, or —CH₂CH₂CH₂—). Similarly, the terms “cycloalkylene”, “heterocycloalkylene”, “arylene”, and “heteroarylene” mean divalent cycloalkyl, heterocycloalkyl, aryl, and heteroaryl, respectively.

As used herein, the term “alkenyl” refers to a linear or branched rnono-unsaturated hydrocarbon chain containing the indicated number of carbon atoms. For example, “(C₂-C₆)alkenyl” refers a linear or branched mono unsaturated hydrocarbon chain of two to six carbon atoms. Non-limiting examples of alkenyl include ethenyl, propenyl, butenyl, or pentenyl.

As used herein, the term “alkynyl” refers to a linear or branched di-unsaturated hydrocarbon chain, containing the indicated number of carbon atoms. For example, “(C₂-C₆)alkynyl” refers to a linear or branched di-unsaturated hydrocarbon chain having two to six carbon atoms. Non-limiting examples of alkynyl include ethynyl, propynyl, butynyl, or pentynyl.

As used herein, the term “cycloalkyl” refers to a saturated or partially unsaturated cyclic hydrocarbon, containing the indicated number of carbon atoms. For example, “(C₃-C₆)cycloalkyl” refers to a saturated or partially unsaturated cyclic hydrocarbon having three to six ring carbon atoms. Non-limiting examples of cycloalkyl include cyclopropyl, cyclobutyl, cyclopentyl, and cyclohexyl. Cycloalkyl may be partially unsaturated. Non-limiting examples of partially unsaturated cycloalkyl include cyclohexenyl, cyclopentenyl, cycloheptenyl, cyclooctenyl, and the like. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like.

As used herein, the term “heterocycloalkyl” refers to a mon-, bi-, tri-, or polycyclic nonaromatic ring system containing indicated number of ring atoms (e.g., 3-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, the heteroatoms selected from O, N, S, or S(O)₁₋₂ (e.g., carbon atoms and 1-3, 1-6, or 1-9 heteroatoms of N, O, S, or S(O)₁₋₂ if monocyclic, bicyclic, or tricyclic, respectively), wherein 0, 1, 2 or 3 atoms of each ring may be substituted by a substituent. Examples of heterocycloalkyl groups include piperazinyl, pyrrolidinyl, dioxanyl, morpholinyl, tetrahydrofuranyl, and the like. Heterocycloalkyl groups may be partially unsaturated. Non-limiting examples of partially unsaturated heterocycloalkyl include dihydropyrrolyl, dihydropyridinyl, tetrahydropyridinyl, dihydrofuranyl, dihydropyranyl, and the like. Heterocycloalkyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocycloalkyl also includes spirocyclic rings (e.g., spirocyclic bicycle wherein two rings are connected through just one atom). Non-limiting examples of spirocyclic heterocycloalkyl include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, the term “aryl” refers to a mono-, bi-, tri- or polycyclic hydrocarbon group containing the indicated numbers of carbon atoms, wherein at least one ring in the system is aromatic (e.g., C₆ monocyclic, C₁₀ bicyclic, or C₁₄ tricyclic aromatic ring system). Examples of aryl groups include phenyl, naphthyl, tetrahydronaphthyl, and the like.

As used herein, the term “heteroaryl” refers to a mono-, bi-, tri- or polycyclic group having indicated numbers of ring atoms (e.g., 5-6 ring atoms; e.g., 5, 6, 9, 10, or 14 ring atoms); wherein at least one ring in the system is aromatic (but does not have to be a ring which contains a heteroatom, e.g. tetrahydroisoquinolinyl, e.g., tetrahydroquinolinyl), and at least one ring in the system contains one or more heteroatoms independently selected from the group consisting of N, O, and S. Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others.

As used herein, the term “haloalkyl” refers to an alkyl radical as defined herein, wherein one or more hydrogen atoms is replaced with one or more halogen atoms. Non-limiting examples include fluoromethyl, difluoromethyl, trifluoromethyl, 2-fluoroethyl, 2,2-difluoroethyl, 2,2,2-trifluoroethyl, chloromethyl, dichloromethyl, chloroethyl, trichloroethyl, bromomethyl, and iodomethyl.

As used herein, the term “alkoxy” refers to an —O-alkyl radical, wherein the radical is on the oxygen atom. For example, “C₁₋₆ alkoxy” refers to an —O—(C₁₋₆ alkyl) radical, wherein the radical is on the oxygen atom. Examples of alkoxy include methoxy, ethoxy, propoxy, isopropoxy, butoxy and tert-butoxy. Accordingly, as used herein, the term “haloalkoxy” refers to an —O-haloalkyl radical, wherein the radical is on the oxygen atom.

As used herein, “ [symbol] ” indicates an optional single or double bond, as allowed by valence. As used herein, “ [symbol] ” indicates the point of attachment to the parent molecule.

As used herein, the term “compound,” is meant to include all stereoisomers, geometric isomers, tautomers, and isotopes of the structures depicted. Compounds herein identified by name or structure as one particular tautomeric form are intended to include other tautomeric forms unless otherwise specified.

As used herein, when a ring is described as being “aromatic”, it means the ring has a continuous, delocalized π-electron system. Typically, the number of out of plane π-electrons corresponds to the Hückel rule (4n+2). Examples of such rings include: benzene, pyridine, pyrimidine, pyrazine, pyridazine, pyridone, pyrrole, pyrazole, oxazole, thioazole, isoxazole, isothiazole, and the like. When a ring system comprising at least two rings is described as “aromatic”, it means the ring system comprises one or more aromatic ring(s). Accordingly, when a ring system comprising at least two rings is described as “non-aromatic”, none of the constituent rings of the ring system is aromatic.

As used herein, when a ring is described as being “partially unsaturated”, it means the ring has one or more additional degrees of unsaturation (in addition to the degree of unsaturation attributed to the ring itself, e.g., one or more double bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like. When a ring system comprising at least two rings is described as “partially unsaturated”, it means the ring system comprises one or more partially unsaturated ring(s), provided that none of the constituent rings of the ring system is aromatic.

As used herein, the term “carboxylic acid bioisostere” means a group which has chemical and physical similarities producing broadly similar biological properties to a carboxylic acid (see Lipinski, Annual Reports in Medicinal Chemistry, 1986, 21, p 283 “Bioisosterism In Drug Design”; Yun, Hwahak Sekye, 1993, 33, pages 576-579 “Application Of Bioisosterism To New Drug Design”; Zhao, Huaxue Tongbao, 1995, pages 34-38 25 “Bioisosteric Replacement And Development Of Lead Compounds In Drug Design”; Graham, Theochem, 1995, 343, pages 105-109 “Theoretical Studies Applied To Drug Design: ab initio Electronic Distributions In Bioisosteres”). Examples of suitable carboxylic acid bioisostere include: sulfo, phosphono, alkylsulfonylcarbamoyl, tetrazolyl, arylsulfonylcarbamoyl, heteroarylsulfonylcarbamoyl, N-methoxycarbamoyl, 3-hydroxy-3-cyclobutene-1,2-dione, 3,5-dioxo-1,2,4-oxadiazolidinyl or heterocyclic phenols such as 3-hydroxyisoxazolyl and 3-hydoxy-1-methylpyrazolyl.

The term “tautomer” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the invention, and the naming of the compounds does not exclude any tautomer.

The term “GLP-1R” or “GLP-1 receptor” as used herein is meant to include, without limitation, nucleic acids, polynucleotides, oligonucleotides, sense and antisense polynucleotide strands, complementary sequences, peptides, polypeptides, proteins, homologous, and/or orthologous GLP-1R molecules, isoforms, precursors, mutants, variants, derivatives, splice variants, alleles, different species, and active fragments thereof.

The term “GLP-1 associated disease” as used herein is meant to include, without limitation, all those diseases, disorders, or conditions in which modulating glucagon-like peptide-1 (GLP-1) receptor signaling can alter the pathology and/or symptoms and/or progression of the disease, disorder, or condition.

The term “GLP-1 agonist” or “GLP-1 RA” as used herein refers to an agonist of the glucagon-like peptide-1 (GLP-1) receptor. GLP-1 RAs enhance glucose-dependent insulin secretion; suppress inappropriately elevated glucagon levels, both in fasting and postprandial states; and slow gastric emptying. Karla et al., Glucagon-like peptide-1 receptor agonists in the treatment of type 2 diabetes: Past, present, and future, Indian J Endocrinol Metab. 2016 March-April; 20(2): 254-267. GLP-1 RAs have been shown to treat type 2 diabetes. Examples of GLP-1 RAs include, but are not limited to, albiglutide (TANZEUM®), dulaglutide (LY2189265, TRULICITY®), efpeglenatide, exenatide (BYETTA®, BYDUREON®, Exendin-4), liraglutide (VICTOZA®, NN2211), lixisenatide (LYXUMIA®), semaglutide (OZEMPIC®), tirzepatide, ZP2929, NNC0113-0987, BPI-3016, and TT401. See, also, for example, additional GLP-1 receptor agonists described in U.S. Pat. Nos. 10,370,426; 10,308,700; 10,259,823; 10,208,019; 9,920,106; 9,839,664; 8,129,343; 8,536,122; 7,919,598; 6,414,126; 6,628,343; and RE45313.

The term “pharmaceutically acceptable” as used herein indicates that the compound, or salt or composition thereof is compatible chemically and/or toxicologically with the other ingredients comprising a formulation and/or the patient being treated therewith.

The term “therapeutic compound” as used herein is meant to include, without limitation, all compounds of Formula I, or pharmaceutically acceptable salts or solvates thereof (e.g., a compound of Formula (I-A2), or a pharmaceutically acceptable salt or solvate thereof), and all compositions (e.g., pharmaceutical compositions) wherein a compound of Formula I, or a pharmaceutically acceptable salt or solvate thereof (e.g., a compound of Formula (I-A2), or a pharmaceutically acceptable salt or solvate thereof) is a component of the composition.

The term “administration” or “administering” refers to a method of giving a dosage of a compound or pharmaceutical composition to a vertebrate or invertebrate, including a mammal, a bird, a fish, or an amphibian. The method of administration can vary depending on various factors, e.g., the components of the pharmaceutical composition, the site of the disease, and the severity of the disease.

The terms “effective amount” or “effective dosage” or “pharmaceutically effective amount” or “therapeutically effective amount,” as used herein, refer to a sufficient amount of a chemical entity (e.g., a compound of Formula I, or a pharmaceutically acceptable salt or solvate thereof (e.g., a compound of Formula (I-A2), or a pharmaceutically acceptable salt or solvate thereof)) being administered which will relieve to some extent one or more of the symptoms of the disease or condition being treated, and can include curing the disease. “Curing” means that the symptoms of active disease are eliminated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. In some embodiments, a “therapeutically effective amount” of a compound as provided herein refers to an amount of the compound that is effective as a monotherapy or combination therapy.

The term “excipient” or “pharmaceutically acceptable excipient” means a pharmaceutically-acceptable material, composition, or vehicle, such as a liquid or solid filler, diluent, carrier, solvent, or encapsulating material. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, P A, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, F L, 2009.

The term “pharmaceutical composition” refers to a mixture of a compound of Formula I, or a pharmaceutically acceptable salt or solvate thereof (e.g., a compound of Formula (I-A2), or a pharmaceutically acceptable salt or solvate thereof) as described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to, rectal, oral, intravenous, aerosol, parenteral, ophthalmic, pulmonary, and topical administration.

The terms “treat,” “treating,” and “treatment,” in the context of treating a disease, disorder, or condition, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof.

The term “preventing”, as used herein, is the prevention of the onset, recurrence or spread, in whole or in part, of the disease or condition as described herein, or a symptom thereof.

The terms “subject”, “patient” or “individual”, as used herein, are used interchangeably and refers to any animal, including mammals such as mice, rats, other rodents, rabbits, dogs, cats, swine, cattle, sheep, horses, primates, and humans. In some embodiments, the term refers to a subject, particularly a mammalian subject, for whom diagnosis, prognosis, or therapy is desired or needed. In some embodiments, the patient is a human. In some embodiments, the subject has experienced and/or exhibited at least one symptom of the disease, disorder, or condition to be treated and/or prevented.

The terms “treatment regimen” and “dosing regimen” are used interchangeably to refer to the dose and timing of administration of each therapeutic agent in a combination of the invention.

The term “pharmaceutical combination”, as used herein, refers to a pharmaceutical treatment resulting from the mixing or combining of more than one active ingredient and includes both fixed and non-fixed combinations of the active ingredients.

The term “combination therapy” as used herein refers to a dosing regimen of two different therapeutically active agents (i.e., the components or combination partners of the combination), wherein the therapeutically active agents are administered together or separately in a manner prescribed by a medical care taker or according to a regulatory agency as defined herein.

The term “modulation”, as used herein, refers to a regulation or an adjustment (e.g., increase or decrease) and can include, for example agonism, partial agonism or antagonism.

### Compounds

Accordingly, provided herein are compounds of Formula I:

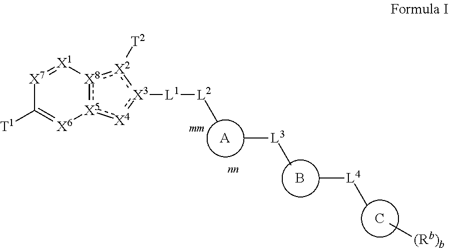

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

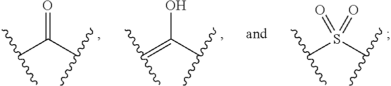

5- to 10-membered heterocycloalkyl, wherein the heterocycloalkyl comprises an endocyclic group selected from the group consisting of:

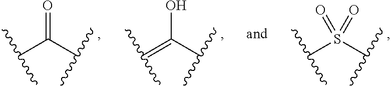

wherein the heterocycloalkyl is optionally substituted with 1-4 R^{v}; and
   (C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

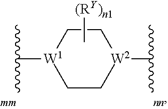

wherein n1 is 0, 1, or 2; W¹ is CR^{Y1} or N; and W² is CR^{Y2} or N;

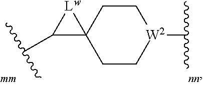

wherein W² is CR^{Y2} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{e} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is selected from the group consisting of: —O—; —S—; —C(R^{a}R^{a})—; —N(H)—; —N(C₁₋₃ alkyl)-; —C(═O)—; and —S(O)₁₋₂—,
each occurrence of R^{a} is independently selected from the group consisting of: hydrogen, halogen, CN, —OH, (C₁-C₆)alkyl, (C₁-C₃)haloalkyl, (C₃-C₈)cycloalkyl, (C₁-C₃)alkoxy, and (C₁-C₃)haloalkoxy; or
a pair of R^{a} taken together with the carbon atom to which each is attached forms a (C₃-C₈)cycloalkyl ring; or
when Ring A is

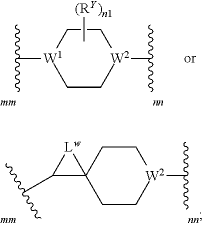

W² is CR^{Y2}; and L³ is —C(R^{a}R^{a})—, one R^{a} combines with R^{Y2} to form a double bond between W² and L³, wherein the remaining R^{a} is selected from the group consisting of: hydrogen, halogen, CN, (C₁-C₆)alkyl, (C₁-C₃)haloalkyl, and (C₃-C₈)cycloalkyl;
Ring B is selected from the group consisting of: (B-I), (B-II), (B-III), (B-IV), and (B-V):

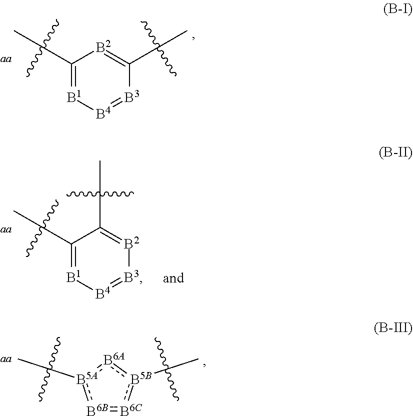

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A} B^{5B}, B^{6A}, B^{6B}, and B^{6C} is C or CR¹, and the ring including B^{5A}, B^{5B}, B^{6A}, B^{6B}, and B^{6C} is heteroaryl;

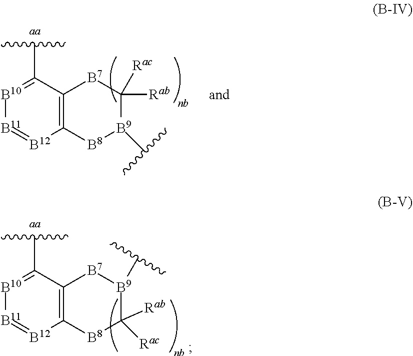

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Further provided herein are compounds of Formula I:

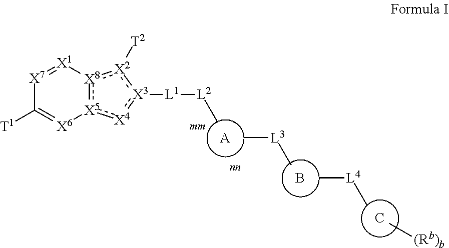

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

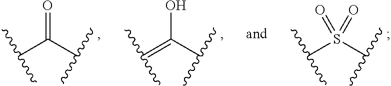

5- to 10-membered heterocycloalkyl, wherein the heterocycloalkyl comprises an endocyclic group selected from the group consisting of:

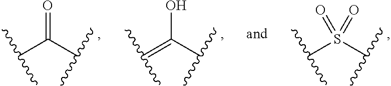

wherein the heterocycloalkyl is optionally substituted with 1-4 R^{v}; and
   (C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
or when q is 2 or 3, a pair of R^{x} on adjacent carbon atoms, taken together, form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, —O(C₁-C₆)alkylene-O(C₁-C₆)alkyl, phenyl, —S(O)₀₋₂—C₁-C₆ alkyl, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

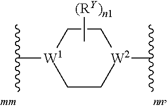

wherein n1 is 0, 1, or 2; W¹ is CR^{Y1} or N; and W² is CR^{Y2} or N;

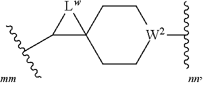

wherein W² is CR^{Y} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};
wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is selected from the group consisting of: —O—; —S—; —C(R^{a}R^{a})—; —N(H)—; —N(C₁₋₃ alkyl)-; —C(═O)—; and —S(O)₁₋₂—,
each occurrence of R^{a} is independently selected from the group consisting of: hydrogen, halogen, CN, —OH, (C₁-C₆)alkyl, (C₁-C₃)haloalkyl, (C₃-C₈)cycloalkyl, (C₁-C₃)alkoxy, and (C₁-C₃)haloalkoxy; or
a pair of R^{a} taken together with the carbon atom to which each is attached forms a (C₃-C₈)cycloalkyl ring; or
when Ring A is

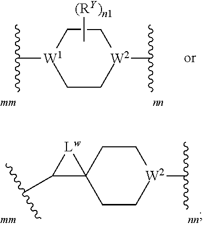

W² is CR^{Y2}; and L³ is —C(R^{a}R^{a})—, one R^{a} combines with R^{Y2} to form a double bond between W² and L³, wherein the remaining R^{a} is selected from the group consisting of: hydrogen, halogen, CN, (C₁-C₆)alkyl, (C₁-C₃)haloalkyl, and (C₃-C₈)cycloalkyl;
Ring B is selected from the group consisting of: (B-I), (B-II), (B-III), (B-IV), and (B-V):

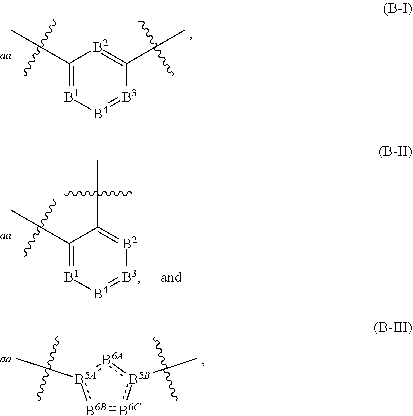

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

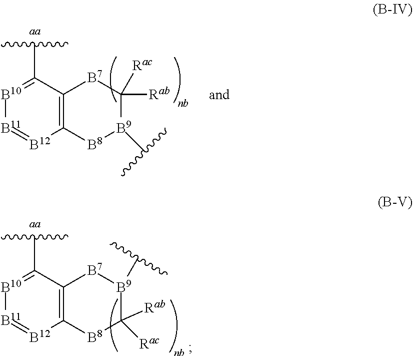

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

Further provided herein are compounds of Formula I:

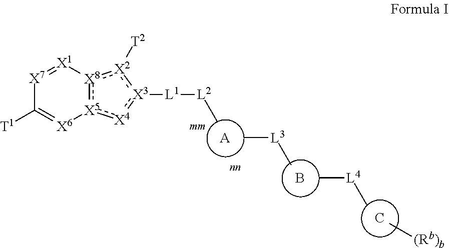

or a pharmaceutically acceptable salt or solvate thereof, wherein:
[symbol] indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
   —N(R^{s})C(═O)R^{z};
   —N(R^{s})C(═O)OR^{z};
   —N(R^{s})C(═O)N(R^{s})R^{z};
   —N(R^{s})S(O)₁₋₂—R^{z};
   —N(R^{s})S(═NR^{s})(═O)R^{z};
   —S(O)₁₋₂R^{z};
   —P(═O)R^{z1}R^{z2};
   —C(═O)OH;
   —C(═O)N(R^{s})R^{z};
   —S(O)₁₋₂N(R^{s})R^{z};
   —S(═NR^{s})(═O)N(R^{s})R^{z};
   5- to 10-membered heteroaryl optionally substituted with 1-4 R^{v}, and wherein the heteroaryl optionally comprises an endocyclic group selected from the group consisting of:

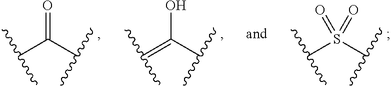

5- to 10-membered heterocycloalkyl, wherein the heterocycloalkyl comprises an endocyclic group selected from the group consisting of:

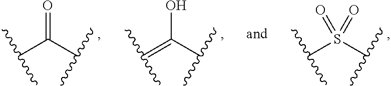

wherein the heterocycloalkyl is optionally substituted with 1-4 R^{v}; and
   (C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
or when q is 2 or 3, a pair of R^{x} on adjacent carbon atoms, taken together, form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, —O(C₁-C₆)alkylene-O(C₁-C₆)alkyl, phenyl, —S(O)₀₋₂—C₁-C₆ alkyl, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of:

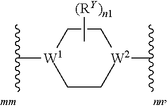

wherein n1 is 0, 1, or 2; W¹ is CR^{Y1} or N; and W² is CR^{Y2} or N;

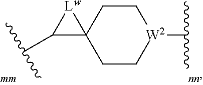

wherein W² is CR^{Y} or N, L^{w} is (C₁-C₃)alkylene;
   phenylene optionally substituted with 1-4 R^{Y};
   5- to 6-membered heteroarylene optionally substituted with 1-3 R^{Y};
   partially unsaturated monocyclic (C₅-C₈)cycloalkylene optionally substituted with 1-4 R^{Y}; and
   partially unsaturated monocyclic 5- to 8-membered heterocycloalkylene optionally substituted with 1-4 R^{Y};

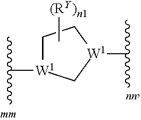

wherein n1 is 0, 1, or 2; W¹ is CR^{Y1} or N; and W² is CR^{Y2} or N; wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is selected from the group consisting of: —O—; —S—; —C(R^{a}R^{a})—; —N(H)—; —N(C₁₋₃ alkyl)-; —C(═O)—; and —S(O)₁₋₂—,
each occurrence of R^{a} is independently selected from the group consisting of: hydrogen, halogen, CN, —OH, (C₁-C₆)alkyl, (C₁-C₃)haloalkyl, (C₃-C₈)cycloalkyl, (C₁-C₃)alkoxy, and (C₁-C₃)haloalkoxy; or
a pair of R^{a} taken together with the carbon atom to which each is attached forms a (C₃-C₈)cycloalkyl ring; or
when Ring A is

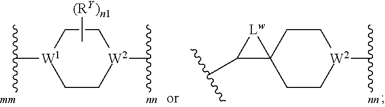

W² is CR^{Y2}; and L³ is —C(R^{a}R^{a})—, one R^{a} combines with R^{Y2} to form a double bond between W² and L³, wherein the remaining R^{a} is selected from the group consisting of: hydrogen, halogen, CN, (C₁-C₆)alkyl, (C₁-C₃)haloalkyl, and (C₃-C₈)cycloalkyl;
Ring B is selected from the group consisting of: (B-I), (B-II), (B-III), (B-IV), and (B-V):

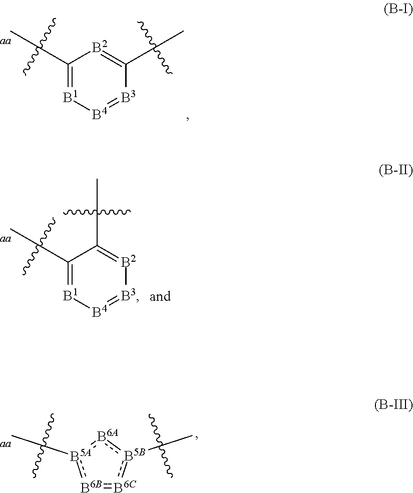

wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl;

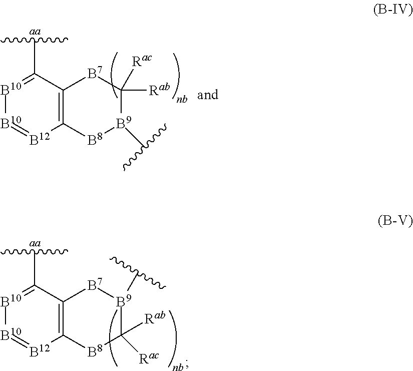

wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

In some embodiments of Formula I, T¹ is -T³. In some embodiments of Formula I, T¹ is -L^{a}-(CR^{x}R^{x})_{q}-T³.

In some embodiments of Formula I, -L^{a} is a bond. In some embodiments of Formula I, -L^{a} is —NH—, —N(C₁₋₃ alkyl)-, —O—, or —S—. As a non-limiting example of the foregoing embodiments, -L^{a} can be —O—.

In some embodiments of Formula I, q is 1. In some embodiments of Formula I, q is 2 or 3.

In some embodiments of Formula I, each R^{x} is hydrogen or (C₁-C₆)alkyl. As a non-limiting example of the foregoing embodiments, each R^{x} can be hydrogen. In some embodiments, one pair of R^{x} on the same carbon, taken together with the carbon atom to which each is attached, forms a (C₃-C₈)cycloalkyl ring. As a non-limiting example of the foregoing embodiments, one pair of R^{x} on the same carbon, taken together with the carbon atom to which each is attached, can form a cyclopropyl. In some embodiments, each remaining R^{x} is hydrogen.

In some embodiments of Formula I, T¹ is —(CR^{x}R^{x})-T³. In some embodiments, each R^{x} is hydrogen. In some embodiments, the pair of R^{x} taken together with the carbon atom to which each is attached, forms a (C₃-C₈)cycloalkyl ring. For example, a pair of R^{x} taken together with the carbon atom to which each is attached, can form a cyclopropyl ring.

In some embodiments of Formula I, T¹ is —(CR^{x}R^{x})_{q}-T³; and q is 2 or 3. In some embodiments, each R^{x} is hydrogen.

In some embodiments of Formula I, T¹ is —O—(CR^{x}R^{x})_{q}-T³; and q is 1, 2, or 3. In some embodiments, q is 1. In some embodiments, q is 2 or 3. In some embodiments, each R^{x} is hydrogen. In some embodiments, q is 1; and each R^{x} is hydrogen.

In some embodiments of Formula I, T³ is selected from the group consisting of: —N(R^{s})C(═O)R^{z}, —N(R^{s})C(═O)OR^{z}, —N(R^{s})C(═O)N(R^{s})R^{z}, —N(R^{s})S(O)₁₋₂—R^{z}, and —N(R^{s})S(═NR^{s})(═O)R^{z}.

In some embodiments of Formula I, T³ is —N(R^{s})C(═O)R^{z}. In some embodiments, T³ is —NHC(═O)R^{z}.

In some embodiments of Formula I, T³ is —N(R^{s})S(O)₁₋₂—R^{z} (e.g., —N(R^{s})S(O)₂—R^{z}). In some embodiments, T³ is —NHS(O)₂R^{z}.

In some embodiments of Formula I, T³ is —N(R^{s})C(═O)N(R^{s})R^{z}. In some embodiments, T³ is —NHC(═O)NHR^{z}.

In some embodiments of Formula I, T³ is —N(R^{s})C(═O)OR^{z}. In some embodiments, T³ is —NHC(═O)OR^{z}.

In some embodiments of Formula I, T³ is —S(O)₁₋₂R^{z}. In some embodiments, T³ is —S(O)₂R^{z}.

In some embodiments of Formula I, T³ is selected from the group consisting of: —C(═O)N(R^{s})R^{z}, —S(O)₁₋₂N(R^{s})R^{z}, and —S(═NR^{s})(═O)N(R^{s})R^{z}.

In some embodiments of Formula I, T³ is —C(═O)N(R^{s})R^{z}. In some embodiments, T³ is —C(═O)NR^{z}.

In some embodiments of Formula I, T³ is —S(O)₂N(R^{s})R^{z}. In some embodiments, T³ is —S(O)₂NR^{z}.

In some embodiments of Formula I, T³ is —S(═NR^{s})(═O)N(R^{s})R^{z}. In some embodiments, T³ is —S(═NH)(═O)N(R^{s})R^{z}. For example, T³ can be —S(═NH)(═O)N(H)R^{z}.

In some embodiments of Formula I, R^{z} is hydrogen.

In some embodiments of Formula I, R^{z} is (C₁-C₆)alkyl optionally substituted with 1-4 R^{v}.

In some embodiments of Formula I, R^{z} is (C₁-C₃)alkyl. As non-limiting examples of the foregoing embodiments, R^{z} can be methyl, ethyl, or isopropyl.

In some embodiments of Formula I, R^{z} is (C₁-C₃)alkyl substituted with 1-3 R^{v}.

In some embodiments, R^{z} is (C₁-C₃)alkyl substituted with from 1-3 substituents each independently selected from the group consisting of halo and (C₁-C₃)alkoxy. For example, R^{z} can be (C₁-C₃)alkyl substituted with 1-3 independently selected halo, such as —CH₂CF₃. As another non-limiting example, R^{z} can be (C₁-C₃)alkyl substituted with (C₁-C₃)alkoxy, such as —CH₂CH₂OMe.

In some embodiments of Formula I, R^{z} is —R^{z3} or -L^{b}-R^{z3}. In some embodiments, R^{z} is —R^{z3} or —CH₂—R^{z3}. In some embodiments, R^{z3} is selected from the group consisting of: (C₃-C₆)cycloalkyl and 3- to 6-membered heterocycloalkyl, each of which is optionally substituted with 1-4 R^{v}. In some embodiments, R^{z3} is selected from the group consisting of: (C₃-C₆)cycloalkyl and 3- to 6-membered heterocycloalkyl, each optionally substituted with 1-2 R^{v}. As non-limiting examples of the foregoing embodiments, R^{z3} can be cyclopropyl, cyclobutyl, difluorocyclobutyl (e.g.,

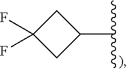 [SMILES: *C1CC(F)(F)C1]

cycopentyl, oxetanyl (e.g.,

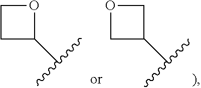 [SMILES: *C1CCO1 ; *C1COC1]

tetrahydropyranyl (e.g.,

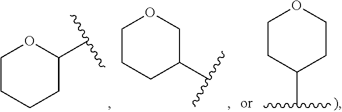

or tetrahydro-2H-thiopyran 1,1-dioxide (e.g.,

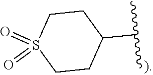 [SMILES: *C1CCS(=O)(=O)CC1]

In some embodiments of Formula I, R^{z3} is selected from the group consisting of: (C₆-C₁₀)aryl and 5- to 10-membered heteroaryl, each of which is optionally substituted with 1-4 R^{v}. In some embodiments, R^{z3} is phenyl optionally substituted with 1-2 R^{v}. For example, R^{z3} can be unsubstituted phenyl.

In some embodiments of Formula I, T³ is —NHC(═O)R^{z} or —NHS(O)₁₋₂—R^{z}; and R^{z} is (C₁-C₆)alkyl optionally substituted with 1-4 R^{v}. In some embodiments, R^{z} is (C₁-C₃)alkyl (e.g., methyl, ethyl, or isopropyl). In some embodiments, R^{z} is (C₁-C₃)alkyl substituted with (C₁-C₃)alkoxy (e.g., —CH₂CH₂OMe). In some embodiments, R^{z} is (C₁-C₃)alkyl substituted with 1-3 halo (e.g., —CH₂CF₃). In some embodiments, T³ is —NHC(═O)R^{z}. In some embodiments, T³ is —NHS(O)₂—R^{z}.

In some embodiments of Formula I, T³ is —NHC(═O)R^{z} or —NHS(O)₁₋₂—R^{z}; and R^{z} is —R^{z3} or —CH₂—R^{z3}. In some embodiments, R^{z3} is selected from the group consisting of: phenyl, (C₃-C₆)cycloalkyl, and 3- to 6-membered heterocycloalkyl, each optionally substituted with 1-2 R^{v}. As non-limiting examples, R^{z3} can be phenyl, cyclopropyl, cyclobutyl, difluorocyclobutyl (e.g.,

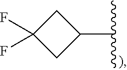 [SMILES: *C1CC(F)(F)C1]

cycopentyl, oxetanyl (e.g.,

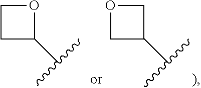 [SMILES: *C1CCO1 ; *C1COC1]

tetrahydropyranyl (e.g.,

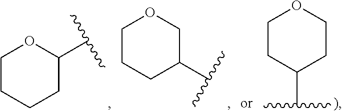

or tetrahydro-2H-thiopyran 1,1-dioxide (e.g.,

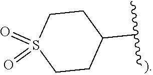 [SMILES: *C1CCS(=O)(=O)CC1]

In some embodiments, T³ is —NHC(═O)R^{z}. In some embodiments, T³ is —NHS(O)₂—R^{z}.

In some embodiments of Formula I, T³ is —S(O)₂R^{z}; and R^{z} is (C₁-C₆)alkyl optionally substituted with 1-4 R^{v}. In some embodiments, R^{z} is (C₁-C₆)alkyl. For example, R^{z} can be (C₁-C₃)alkyl

In some embodiments of Formula I, T³ is selected from the group consisting of: —C(═O)N(R^{s})R^{z}, —S(O)₁₋₂N(R^{s})R^{z}, and —S(═NR^{s})(═O)N(R^{s})R^{z}; and R^{z} is hydrogen or (C₁-C₆)alkyl optionally substituted with 1-4 R^{v}. In some embodiments, T³ is —C(═O)N(R^{s})R^{z}. For example, T³ can be —C(═O)NHR^{z}. In some embodiments, T³ is —S(O)₂N(R^{s})R^{z}. For example, T³ can be —S(O)₂NHR^{z}. In some embodiments, T³ is —S(═NR^{s})(═O)N(R^{s})R^{z}. For example, T³ can be —S(═NH)(═O)N(R^{s})R^{z} (e.g., —S(═NH)(═O)N(H)R^{z}). In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula I, T³ is —N(R^{s})C(═O)N(R^{s})R^{z} or —N(R^{s})C(═O)OR^{z}; and R^{z} is hydrogen or (C₁-C₆)alkyl optionally substituted with 1-4 R^{v}. In some embodiments, T³ is —NHC(═O)NHR^{z}. In some embodiments, T³ is —N(H)C(═O)OR^{z}. In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula I, T³ is —C(═O)OH.

In some embodiments of Formula I, T³ is —P(═O)R^{z1}R^{z2}. In some embodiments, R^{z1} and R^{z2} are each independently selected (C₁-C₃)alkyl. For example, each R^{z1} can be independently methyl or ethyl.

In some embodiments of Formula I, T³ is 5- to 10-membered heteroaryl optionally substituted with 1-4 R^{v}, and wherein the heteroaryl optionally comprises an endocyclic group selected from the group consisting of:

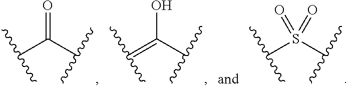

In some embodiments of Formula I, T³ is 5- to 6-membered heteroaryl optionally substituted with 1-4 R^{v}. In some embodiments, T³ is 5-membered heteroaryl having 2-4 ring heteroatoms selected from the group consisting of N, O, and S, wherein the heteroaryl is optionally substituted with 1-2 R^{v}. As non-limiting examples of the foregoing embodiments, T³ can be selected from the group consisting of:

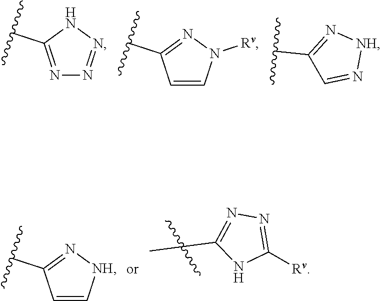

In some embodiments

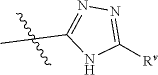

can be

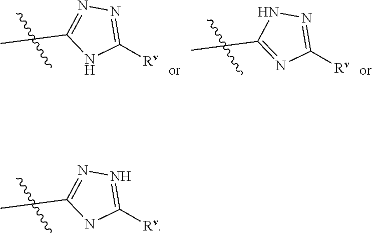

In some embodiments of Formula I, T³ is 5- to 6-membered heteroaryl that comprises an endocyclic group selected from the group consisting of:

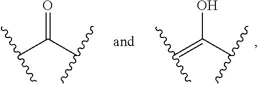

wherein the heteroaryl is further optionally substituted with 1-4 R^{v}. As non-limiting examples of the foregoing embodiments, T³ can be selected from the group consisting of:

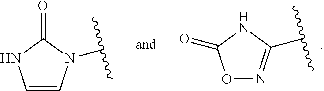 [SMILES: *n1cc[nH]c1=O ; *c1noc(=O)[nH]1]

In some embodiments of Formula I, T³ is 5- to 10-membered heterocycloalkyl, wherein the heterocycloalkyl comprises an endocyclic group selected from the group consisting of:

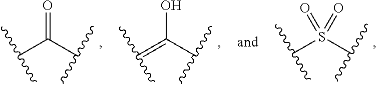

wherein the heterocycloalkyl is optionally substituted with 1-4 R^{v}. In some embodiments, T³ is 5- to 6-membered heterocycloalkyl which comprises an endocyclic group selected from the group consisting of:

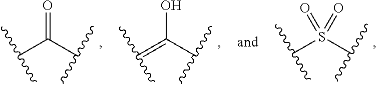

wherein the heterocycloalkyl is optionally substituted with 1-4 R^{v}.

In some embodiments, T³ is

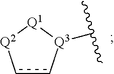

Q¹ is C(═O) or S(O)₂; Q² is O, NH, —CH₂—, or —CH₂—CH₂—; Q³ is N or CH; and [symbol] is a single bond or a double bond, provided that T³ is non-aromatic. As non-limiting examples of the foregoing embodiments, T³ can be selected from the group consisting of:

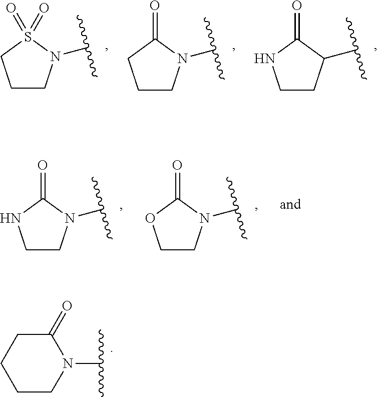

In some embodiments of Formula I, T³ is (C₁-C₆)haloalkyl substituted with —OH and further optionally substituted with 1-2 R^{v}. In some embodiments, T³ is (C₁-C₆)haloalkyl substituted with —OH and further optionally substituted with 1-2 R^{v}. In some embodiments, T³ is (C₁-C₆)haloalkyl substituted with —OH. As a non-limiting example of the foregoing embodiments, T¹ can be

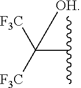 [SMILES: *C(O)(C(F)(F)F)C(F)(F)F]

In some embodiments of Formula I, T¹ is —N(R^{s})C(═O)R^{z} or —N(R^{s})S(O)₁₋₂—R^{z}; and R^{z} is (C₁-C₆)alkyl optionally substituted with 1-4 R^{v}. In some embodiments, R^{z} is (C₁-C₃)alkyl (e.g., methyl, ethyl, or isopropyl). In some embodiments, R^{z} is (C₁-C₃)alkyl substituted with from 1-3 substituents each independently selected from the group consisting of halo and (C₁-C₃)alkoxy. For example, R^{z} can be —CH₂CF₃ or —CH₂CH₂OMe. In some embodiments, T¹ is —NHC(═O)R^{z}. In some embodiments, T¹ is —NHS(O)₂—R^{z}.

In some embodiments of Formula I, T¹ is —N(R^{s})C(═O)R^{z} or —N(R^{s})S(O)₁₋₂—R^{z}; and R^{z} is —R^{z3} or —CH₂—R^{z3}. In some embodiments, R^{z3} is selected from the group consisting of: phenyl, (C₃-C₆)cycloalkyl, and 3- to 6-membered heterocycloalkyl, each optionally substituted with 1-2 R^{v}. As non-limiting examples of the foregoing embodiments, R^{z3} can be phenyl, cyclopropyl, cyclobutyl, difluorocyclobutyl (e.g.,

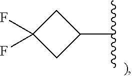 [SMILES: *C1CC(F)(F)C1]

cycopentyl, oxetanyl (e.g.,

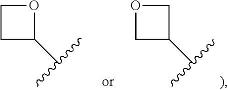 [SMILES: *C1CCO1 ; *C1COC1]

or tetrahydropyranyl (e.g.,

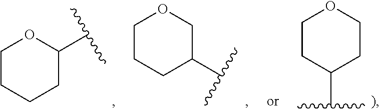 [SMILES: *C1CCCCO1 ; *C1CCCOC1 ; *C1CCOCC1]

or tetrahydro-2H-thiopyran 1,1-dioxide (e.g.,

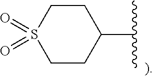 [SMILES: *C1CCS(=O)(=O)CC1]

In some embodiments, T¹ is —NHC(═O)R^{z}. In some embodiments, T¹ is —NHS(O)₂—R^{z}.

In some embodiments of Formula I, T¹ is —(CR^{x}R^{x})_{q}—S(O)₂R^{z}; and q is 1, 2, or 3. In some embodiments, R^{z} is (C₁-C₆)alkyl optionally substituted with 1-4 R^{v}. In some embodiments, R^{z} is (C₁-C₃)alkyl. In some embodiments, q is 1. In some embodiments, q is 2. In some embodiments, each R^{x} is hydrogen.

In some embodiments of Formula I, T¹ is selected from the group consisting of: —C(═O)N(R^{s})R^{z}, —S(O)₁₋₂N(R^{s})R^{z}, and —S(═NR^{s})(═O)N(R^{s})R^{z}. In some embodiments, T¹ is —C(═O)N(R^{s})R^{z}. For example, T¹ can be —C(═O)NR^{z}. In some embodiments, T¹ is —S(O)₂N(R^{s})R^{z}. For example, T¹ can be —S(O)₂NR^{z}. In some embodiments, T is —S(═NR^{s})(═O)N(R^{s})R^{z}. For example, T¹ can be —S(═NH)(═O)N(R^{s})R^{z} (e.g., —S(═NH)(═O)N(H)R^{z}). In some embodiments, R^{z} is hydrogen or (C₁-C₆)alkyl optionally substituted with 1-4 R^{v}. In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula I, T is —N(R^{s})C(═O)N(R^{s})R^{z} or —N(R^{s})C(═O)OR^{z}. In some embodiments, T¹ is —NHC(═O)NR^{z}. In some embodiments, T¹ is —N(H)C(═O)OR^{z}. In some embodiments, R^{z} is hydrogen or (C₁-C₆)alkyl optionally substituted with 1-4 R^{v} In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula I, T¹ is —(CR^{x}R^{x})_{q}—C(═O)OH; and q is 1, 2, or 3. In some embodiments, q is 1. In some embodiments, q is 2. In some embodiments, each R^{x} is hydrogen. In some embodiments (e.g., when q is 1), one pair of R^{x} on the same carbon, taken together with the carbon atom to which each is attached, forms a (C₃-C₈)cycloalkyl ring; and each remaining R^{x} when present is hydrogen. As a non-limiting example of the foregoing embodiments, the pair R^{x} on the same carbon, taken together with the carbon atom to which each is attached, can form a cyclopropyl ring. As non-limiting examples of the foregoing embodiments, T¹ can be

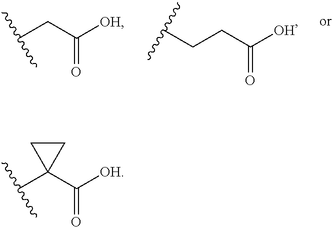 [SMILES: *CC(=O)O ; *CCC(=O)O ; *C1(C(=O)O)CC1]

In some embodiments of Formula I, T¹ is —C(═O)OH.

In some embodiments of Formula I, T¹ is —O—(CR^{x}R^{x})_{q}—C(═O)OH; and q is 1, 2, or 3. In some embodiments, q is 1. In some embodiments, each R^{x} is hydrogen.

In some embodiments of Formula I, T¹ is —P(═O)R^{z1}R^{z2}. In some embodiments, R^{z1} and R^{z2} are independently selected (C₁-C₃)alkyl. For example, R^{z1} and R^{z2} can be independently methyl or ethyl.

In some embodiments of Formula I, T¹ is 5-membered heteroaryl having 2-4 ring heteroatoms selected from the group consisting of N, O, and S, wherein the heteroaryl is optionally substituted with 1-2 R^{v}. As non-limiting examples of the foregoing embodiments, T¹ can be selected from the group consisting of:

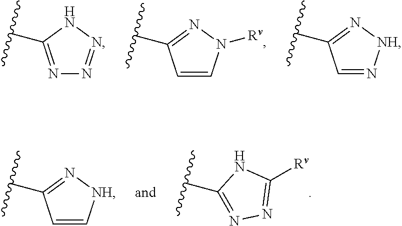

In some embodiments of Formula I, T¹ is 5- to 6-membered heteroaryl that comprises an endocyclic group selected from the group consisting of:

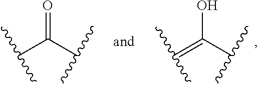

wherein the heteroaryl is further optionally substituted with 1-4 R^{v}. As non-limiting examples of the foregoing embodiments, T¹ can be selected from the group consisting of:

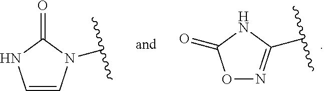 [SMILES: *n1cc[nH]c1=O ; *c1noc(=O)[nH]1]

In some embodiments, T¹ is

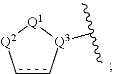

Q¹ is C(═O) or S(O)₂; Q² is O, NH, —CH₂—, or —CH₂—CH₂—; Q³ is N or CH; and [symbol] is a single bond or a double bond, provided that T¹ is non-aromatic. As non-limiting examples of the foregoing embodiments, T¹ can be selected from the group consisting of:

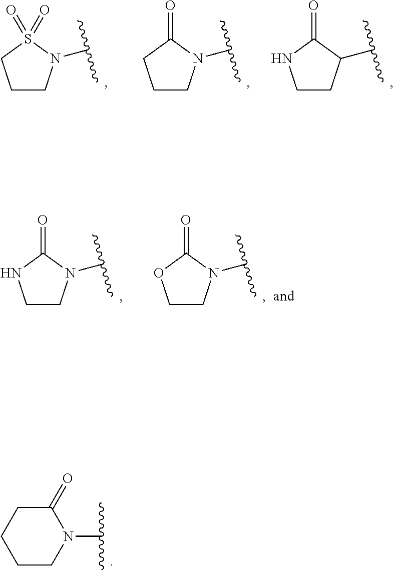 [SMILES: *N1CCCS1(=O)=O ; *N1CCCC1=O ; *C1CCNC1=O ; *N1CCNC1=O ; *N1CCOC1=O ; *N1CCCCC1=O]

In some embodiments of Formula I, T¹ is (C₁-C₆)haloalkyl substituted with —OH and further optionally substituted with 1-2 R^{v}. In some embodiments, T¹ is (C₁-C₆)haloalkyl substituted with —OH. As a non-limiting example of the foregoing embodiments, T¹ can be

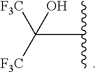 [SMILES: *C(O)(C(F)(F)F)C(F)(F)F]

In some embodiments of Formula I, X² is N; and X⁴ is N.

In some embodiments of Formula I, X⁸ is C; and X⁵ is C.

In some embodiments of Formula I, X³ is C.

In some embodiments of Formula I, X² is N; X³ is C; X⁴ is N; X⁵ is C; and X⁸ is C.

In some embodiments of Formula I, each of X¹, X⁷, and X⁶ is independently CH or CR^{w}. In some embodiments, X² is N; X³ is C; X⁴ is N; X⁵ is C; and X⁸ is C.

In some embodiments of Formula I, each of X¹, X⁷, and X⁶ is CH. In some embodiments, X² is N; X³ is C; X⁴ is N; X⁵ is C; and X⁸ is C.

In some embodiments of Formula I, one of X¹, X⁷, and X⁶ is CR^{w}; and each remaining of X¹, X⁷, and X⁶ is CH. In some embodiments, X² is N; X³ is C; X⁴ is N; X⁵ is C; and X⁸ is C.

In some embodiments of Formula I, X⁶ is CR^{w}; and X¹ and X⁷ are CH. As a non-limiting example of the foregoing embodiments, X⁶ can be C—F. In some embodiments, X² is N; X³ is C; X⁴ is N; X⁵ is C; and X⁸ is C.

In some embodiments of Formula I, one of X¹, X⁷, and X⁶ is N; and each remaining of X¹, X⁷, and X⁶ is CH or CR^{w}. In some embodiments of Formula I, X¹ is N; and X⁶ and X⁷ are CH. In some embodiments of Formula I, X⁶ is N; and X¹ and X⁷ are CH. In some embodiments of Formula I, X⁷ is N; and X¹ and X⁶ are CH. In some embodiments, X² is N; X³ is C; X⁴ is N; X⁵ is C; and X⁸ is C.

In some embodiments of Formula I, the

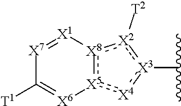

moiety is

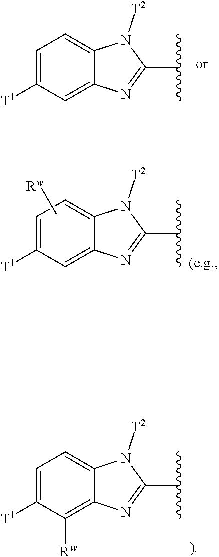

For example, the

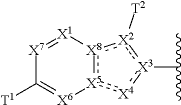

moiety can be

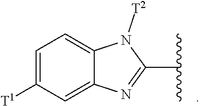

As another non-limiting example, the

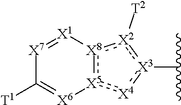

moiety can be

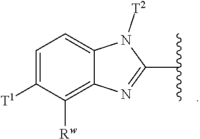

In some embodiments of Formula I, the

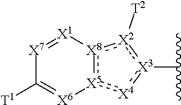

moiety is

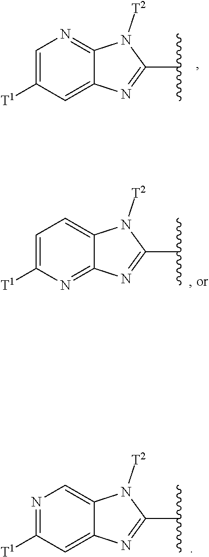

For example, the

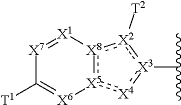

moiety can be

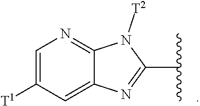

As another non-limiting example, the

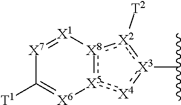

moiety can be

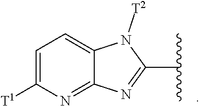

As another non-limiting example, the

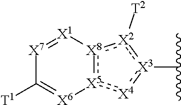

moiety can be

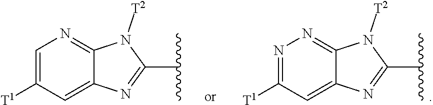

In some embodiments of Formula I, T² is (C₁-C₆)alkyl which is substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

In some embodiments of Formula I, T² is (C₁-C₆)alkyl which is substituted with 3- to 6-membered heterocycloalkyl. In some embodiments of Formula I, T² is (C₁-C₃)alkyl which is substituted with 4- to 6-membered heterocycloalkyl. In some embodiments of Formula I, T² is (C₁-C₃)alkyl which is substituted with oxetanyl. As a non-limiting example of the foregoing embodiments, T² can be

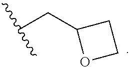 [SMILES: *CC1CCO1]

For example, T² can be

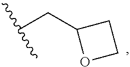 [SMILES: *CC1CCO1]

wherein the stereogenic center in T² has (S)-configuration.

In some embodiments of Formula I, L¹ is (C₁-C₃)alkylene which is optionally substituted with 1-3 R^{L}. As a non-limiting example of the foregoing embodiments, L¹ can be CH₂.

In some embodiments of Formula I, L¹ is a bond.

In some embodiments of Formula I, L² is a bond.

In some embodiments of Formula I, L¹ is CH₂; and L² is a bond.

In some embodiments of Formula I, L¹ is a bond; and L² is a bond. For avoidance of doubt, when L¹ is a bond; and L² is a bond, X³ is directly attached to Ring A.

In some embodiments of Formula I, Ring A is

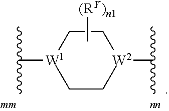

In some embodiments, W¹ is N. In some embodiments, W² is CR^{Y2}. In some embodiments, R^{Y2} is hydrogen. For example, W² can be CH. In some embodiments, W² is N. In some embodiments, n1 is 0. In some embodiments, n1 is 1.

As a non-limiting example of the foregoing embodiments, Ring A can be

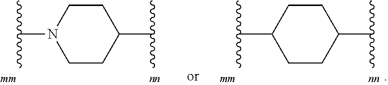 [SMILES: *C1CCN(*)CC1 ; *C1CCC(*)CC1]

In some embodiments of Formula I, Ring A is

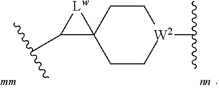

In some embodiments, L^{w} is CH₂. In some embodiments, W² is N. As a non-limiting example of the foregoing embodiments, Ring A can be

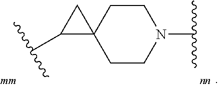 [SMILES: *C1CC12CCN(*)CC2]

In some embodiments of Formula I, Ring A is selected from the group consisting of:
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y}.

In some embodiments of Formula I, Ring A is

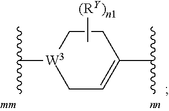

W³ is N or CH; and n1 is 0, 1, or 2. In some embodiments, n1 is 0. In some embodiments, W³ is N. In some embodiments, W³ is CH. As a non-limiting example of the foregoing embodiments, Ring A can be

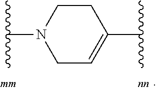 [SMILES: *C1=CCN(*)CC1]

As a further non-limiting example of the foregoing embodiments, Ring A can be

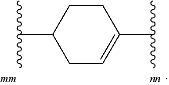 [SMILES: *C1=CCC(*)CC1]

In some embodiments of Formula I, Ring A is

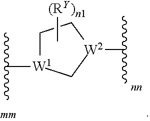

In some embodiments, W¹ is N. In some embodiments, W² is CR^{Y2}. In some embodiments, R^{Y2} is hydrogen. For example, W² can be CH. As a non-limiting example of the foregoing embodiments, Ring A can be

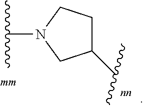 [SMILES: *C1CCN(*)C1]

In some embodiments of Formula I, L² is a bond; L¹ is CH₂; and Ring A is

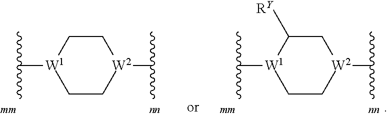

As a non-limiting example of the foregoing disclosure, Ring A can be

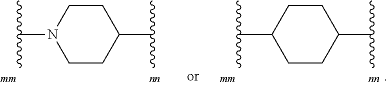 [SMILES: *C1CCN(*)CC1 ; *C1CCC(*)CC1]

In some embodiments of Formula I, L² is a bond; L¹ is CH₂; and Ring A is

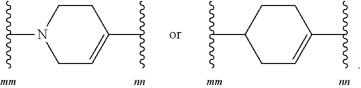 [SMILES: *C1=CCN(*)CC1 ; *C1=CCC(*)CC1]

In some embodiments of Formula I, L² is a bond; L¹ is a bond; and Ring A is

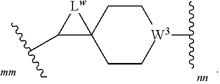

As a non-limiting example of the foregoing embodiments, Ring A can be

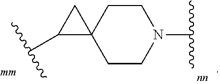 [SMILES: *C1CC12CCN(*)CC2]

In some embodiments of Formula I, L³ is —O—.

In some embodiments of Formula I, B⁴ is CR¹ (e.g., CH). In some embodiments of Formula I, B⁴ is N.

In some embodiments of Formula I, B is CR¹ (e.g., CH). In some embodiments of Formula I, B¹ is N.

In some embodiments of Formula I, B³ is CR¹ (e.g., CH). In some embodiments of Formula I, B³ is N.

In some embodiments of Formula I, B² is CR¹ (e.g., CH). In some embodiments of Formula I, B² is N.

In some embodiments of Formula I, Ring B is

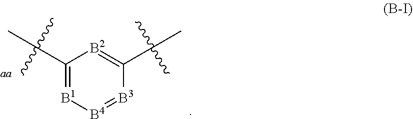

In some embodiments, B² is N. In some embodiments, B⁴ is CR¹ (e.g., CH). In some embodiments, B¹ is CR¹ (e.g., CH). In some embodiments, B³ is CR¹ (e.g., CH). In some embodiments, B² is N; and B¹, B³, and B⁴ are independently CR¹. For example, B² can be N; and B¹, B³, and B⁴ can each be CH.

In some embodiments of Formula I, Ring B is

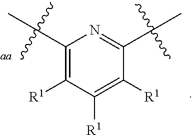

As a non-limiting example of the foregoing embodiments, Ring B can be

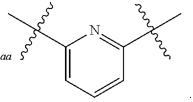 [SMILES: *c1cccc(*)n1]

In some embodiments of Formula I, Ring B is

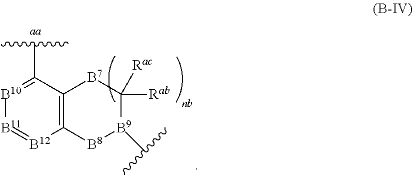

In some embodiments, B⁹ is CR^{aa} (e.g., CH or C(C₁-C₃ alkyl) such as CMe). In some embodiments, B⁷ is —O—. In some embodiments, B⁸ is —O—. In some embodiments, B⁷ is —O—; and B⁸ is —O—. In some embodiments, nb is 1. In some embodiments, R^{ab} is H. In some embodiments, R^{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, nb is 0. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B¹² can be CH. In some embodiments, B¹⁰, B¹¹, and B¹² are independently selected CR¹. As a non-limiting example of the foregoing embodiments, B¹⁰, B¹¹, and B¹² are each CH.

In some embodiments of Formula I, Ring B is

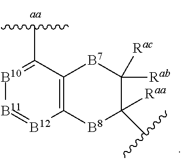

In some embodiments, B⁷ is —O—. In some embodiments, B⁸ is —O—. In some embodiments, B⁷ is —O—; and B⁸ is —O—. In some embodiments, R^{aa} is H. In some embodiments, R^{aa} is (C₁-C₃)alkyl. As a non-limiting example of the foregoing embodiments, R^{aa} can be methyl. In some embodiments, R^{ab} is H. In some embodiments, R^{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B¹² can be CH. In some embodiments, B¹⁰, B¹¹ and B¹² are independently selected CR¹. As a non-limiting example of the foregoing embodiments, B¹⁰, B¹¹, and B¹² are each CH. In some embodiments, the carbon atom to which L⁴ and R^{aa} are both attached has (R)-configuration. In some embodiments, the carbon atom to which L⁴ and R^{aa} are both attached has (S)-configuration.

In some embodiments of Formula I, Ring B is

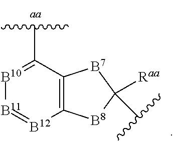

In some embodiments, B⁷ is —O—. In some embodiments, B⁸ is —O—. In some embodiments, B⁷ is —O—; and B⁸ is —O—. In some embodiments, R^{aa} is H. In some embodiments, R^{aa} is (C₁-C₃)alkyl. As a non-limiting example of the foregoing embodiments, R^{aa} can be methyl. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B¹² can be CH. In some embodiments, B¹⁰, B¹¹, and B¹² are independently selected CR¹. As a non-limiting example of the foregoing embodiments, B¹⁰, B¹¹, and B¹² are each CH. In some embodiments, the carbon atom to which L⁴ and R^{aa} are both attached has (R)-configuration. In some embodiments, the carbon atom to which L⁴ and R^{aa} are both attached has (S)-configuration.

In some embodiments of Formula I, Ring B is

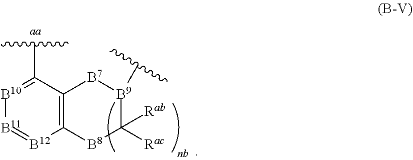

In some embodiments, B⁹ is CR^{aa} (e.g., CH or C(C₁-C₃ alkyl) such as CMe). In some embodiments, B⁷ is —O—. In some embodiments, B⁸ is —O—. In some embodiments, B⁷ is —O—; and B⁸ is —O—. In some embodiments, nb is 1. In some embodiments, R^{ab} is H. In some embodiments, R^{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, nb is 0. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B¹² can be CH. In some embodiments, B¹⁰, B¹¹, and B¹² are independently selected CR¹. As a non-limiting example of the foregoing embodiments, B¹⁰, B¹¹, and B¹² are each CH.

In some embodiments of Formula I, Ring B is

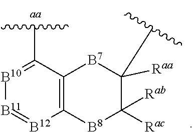

In some embodiments, B⁷ is —O—. In some embodiments, B⁸ is —O—. In some embodiments, B⁷ is —O—; and B⁸ is —O—. In some embodiments, R^{aa} is H. In some embodiments, R^{aa} is (C₁-C₃)alkyl. As a non-limiting example of the foregoing embodiments, R^{aa} can be methyl. In some embodiments, R^{ab} is H. In some embodiments, R_{ac} is H. In some embodiments, R^{aa}, R^{ab}, and R^{ac} are each H. In some embodiments, R^{aa} is (C₁-C₃)alkyl; and R^{ab} and R^{ac} are H. In some embodiments, B¹⁰ is CR¹. As a non-limiting example of the foregoing embodiments, B¹⁰ can be CH. In some embodiments, B¹¹ is CR¹. As a non-limiting example of the foregoing embodiments, B¹¹ can be CH. In some embodiments, B¹² is CR¹. As a non-limiting example of the foregoing embodiments, B¹² can be CH. In some embodiments, B¹⁰, B¹¹, and B¹² are independently selected CR¹. As a non-limiting example of the foregoing embodiments, B¹⁰, B¹¹, and B¹² are each CH. In some embodiments, the carbon atom to which L⁴ and R^{aa} are both attached has (R)-configuration. In some embodiments, the carbon atom to which L⁴ and R^{aa} are both attached has (S)-configuration.

In some embodiments of Formula I, Ring B is

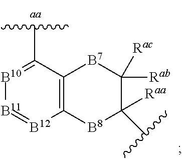

B⁷ and B⁸ are —O—; and R^{aa} is H or (C₁-C₃)alkyl. In some embodiments, B¹⁰, B¹¹, and B¹² are each independently selected CR¹. As non-limiting examples of the foregoing embodiments, B¹⁰, B¹¹, and B¹² can be CH. In some embodiments, R^{aa} is H. In some embodiments, R^{aa} is (C₁-C₃)alkyl (e.g., methyl). In some embodiments, R^{ab} and R^{ac} are H.

In some embodiments of Formula I, Ring B is

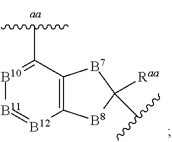

B⁷ and B⁸ are —O—; and R^{aa} is H or (C₁-C₃)alkyl. In some embodiments, B¹⁰, B¹¹, and B¹² are each independently selected CR¹. As non-limiting examples of the foregoing embodiments, B¹⁰, B¹¹, and B¹² can be CH. In some embodiments, R^{aa} is H. In some embodiments, R^{aa} is (C₁-C₃)alkyl (e.g., methyl).

In some embodiments of Formula I, Ring B is

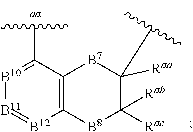

B⁷ and B⁸ are —O—; and R^{aa} is H or (C₁-C₃)alkyl. In some embodiments, B¹⁰, B¹¹, and B¹² are each independently selected CR¹. As non-limiting examples of the foregoing embodiments, B¹⁰, B¹¹, and B¹² can be CH. In some embodiments, R^{aa} is H. In some embodiments, R^{aa} is (C₁-C₃)alkyl (e.g., methyl). In some embodiments, R^{ab} and R^{ac} are H.

As non-limiting examples, Ring B can be

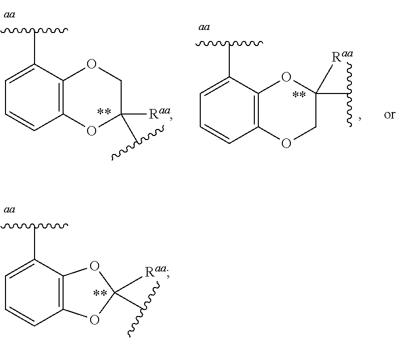

and the carbon atom labelled with ** has (R)-configuration.

As another non-limiting example, Ring B can be

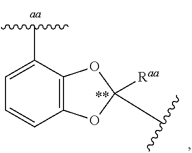

and the carbon atom labelled with ** has (S)-configuration.

In some embodiments of Formula I, L⁴ is a bond.

In some embodiments of Formula I, Ring B is (B-IV) or (B-V); and L⁴ is a bond.

In some embodiments of Formula I, L⁴ is —Z¹—Z²—*, wherein * represents the point of attachment to Ring C.

In some embodiments of Formula I, Z¹ is —O—. In some embodiments of Formula I, Z¹ is a bond.

In some embodiments of Formula I, Z² is —CH₂— optionally substituted with 1-2 R^{c}. As a non-limiting example of the foregoing embodiments, Z² can be —CH₂—.

In some embodiments, L⁴ is —O—CH₂—*. In some embodiments, L⁴ is —CH₂—.

In some embodiments of Formula I, L⁴ is a bond.

In some embodiments of Formula I, L⁴ is —O—Z²—*, wherein * represents the point of attachment to Ring C; and Z² is —CH₂— optionally substituted with 1-2 R^{c}.

In some embodiments of Formula I, L³ is —O—; and L⁴ is —CH₂—.

In some embodiments of Formula I, Ring C is selected from the group consisting of: phenyl, 5- to 6-membered heteroaryl, and 5- to 10-membered bicycloheteroaryl. In some embodiments, Ring C is selected from the group consisting of: phenyl and 6-membered heteroaryl. As a non-limiting example of the foregoing embodiments, Ring C can be phenyl. As another non-limiting example, Ring C can be a 6-membered heteroaryl (e.g., pyridyl).

In some embodiments of Formula I, b is 1-3. In some embodiments, b is 2. In some embodiments, b is 1. In some embodiments, b is 0.

In some embodiments of Formula I, Ring C is phenyl; and b is 2.

In some embodiments of Formula I, Ring C is phenyl; and b is 1.

In some embodiments of Formula I, Ring C is phenyl; and b is 0.

In some embodiments of Formula I, Ring C is a 6-membered heteroaryl (e.g., pyridyl); and b is 1 or 2 (e.g., 1).

In some embodiments of Formula I,

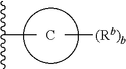

is

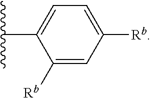

In some embodiments, each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN. In some embodiments, each occurrence of R^{b} is independently selected from the group consisting of —F, —Cl, CF₃, and CN.

In some embodiments of Formula I,

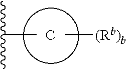

is

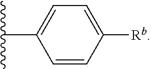

In some embodiments, R^{b} is independently selected from the group consisting of: (C₁-C₆)alkyl, (C₁-C₆)haloalkyl, (C₁-C₆)alkoxy, (C₁-C₆)haloalkoxy, halogen, and CN. In some embodiments, R^{b} is independently selected from the group consisting of —F, —Cl, CF₃, and CN.

In some embodiments of Formula I,

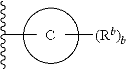

is

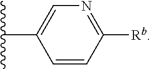

In some embodiments, each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN. In some embodiments, each occurrence of R^{b} is independently selected from the group consisting of —F, —Cl, CF₃, and CN.

In some embodiments, the compound of Formula I is a compound of Formula (I-A2) or a pharmaceutically acceptable salt thereof:

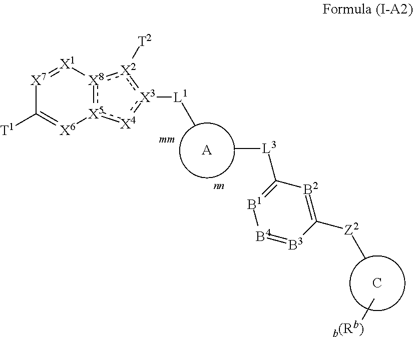

wherein L³ is selected from the group consisting of: —O—; —S—; —C(R^{a}R^{a})—; —N(H)—; —N(C₁₋₃ alkyl)-; —C(═O)—; and —S(O)₁₋₂—; and
Z² is selected from the group consisting of: NH, N(C₁-C₆ alkyl), O, C(═O), S(O)₀₋₂, and —CR^{cA}R^{cB}—,
wherein R^{cA} and R^{cB} are independently selected from the group consisting of H and R^{c}.

In some embodiments Formula (I-A2), L³ is —O—.

In some embodiments Formula (I-A2), Z² is —CR^{cA}R^{cB}—. As a non-limiting example of the forgoing embodiments, Z² can be —CH₂—.

In some embodiments Formula (I-A2), the ring containing B¹, B², B³, and B⁴ is

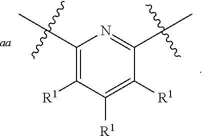

As a non-limiting example of the foregoing embodiments, the ring containing B¹, B², B³, and B⁴ can be

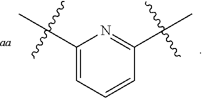 [SMILES: *c1cccc(*)n1]

In some embodiments of Formula (I-A2), the

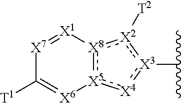

moiety is

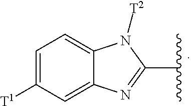

In some embodiments of Formula (I-A2), the

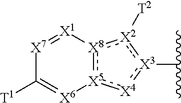

moiety is

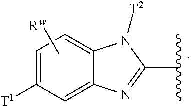

In some embodiments of Formula (I-A2), the

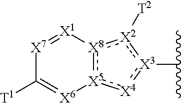

moiety is

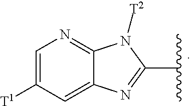

In some embodiments of Formula (I-A2), the

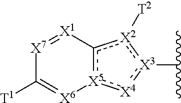

moiety is

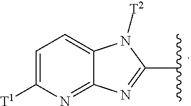

In some embodiments of Formula (I-A2), the

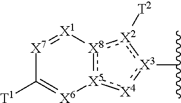

moiety is

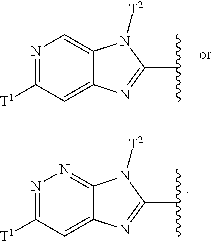

In some embodiments of Formula (I-A2), T¹ is —NR^{s}C(═O)R^{z} or —NR^{s}S(O)₁₋₂—R^{z}; and R^{z} is (C₁-C₆)alkyl optionally substituted with 1-4 R^{v}. In some embodiments, R^{z} is (C₁-C₃)alkyl (e.g., methyl, ethyl, or isopropyl). In some embodiments, R^{z} is (C₁-C₃)alkyl substituted with (C₁-C₃)alkoxy (e.g., —CH₂CH₂OMe). In some embodiments, R^{z} is (C₁-C₃)alkyl substituted with 1-3 independently selected halo (e.g., —CH₂CF₃).

In some embodiments of Formula (I-A2), T¹ is —NR^{s}C(═O)R^{z} or —NR^{s}S(O)₁₋₂—R^{z}; and R^{z} is —R^{z3} or —CH₂—R^{z3}. In some embodiments, R^{z3} is selected from the group consisting of: phenyl, (C₃-C₆)cycloalkyl, and 3- to 6-membered heterocycloalkyl, each optionally substituted with 1-2 R^{v}. For example, R^{z3} can be R^{z3} can be phenyl, cyclopropyl, cyclobutyl, difluorocyclobutyl (e.g.,

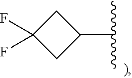 [SMILES: *C1CC(F)(F)C1]

cycopentyl, oxetanyl (e.g.,

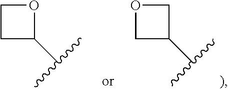 [SMILES: *C1CCO1 ; *C1COC1]

tetrahydropyranyl (e.g.,

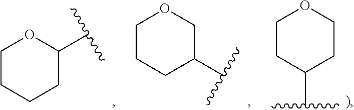 [SMILES: *C1CCCCO1 ; *C1CCCOC1 ; *C1CCOCC1]

or tetrahydro-2H-thiopyran 1,1-dioxide (e.g.,

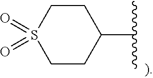 [SMILES: *C1CCS(=O)(=O)CC1]

In some embodiments, T³ is —NHC(═O)R^{z}. In some embodiments, T³ is —NHS(O)₂—R^{z}.

In some embodiments of Formula (I-A2), T¹ is —(CR^{x}R^{x})_{q}—S(O)₂R^{z}; and q is 1, 2, or 3. In some embodiments, R^{z} is (C₁-C₆)alkyl optionally substituted with 1-4 R^{v}. In some embodiments of Formula, R^{z} is (C₁-C₃)alkyl. In some embodiments, q is 1. In some embodiments, q is 2 or 3. In some embodiments, each R^{x} is hydrogen. In some embodiments, q is 1; and each R^{x} is hydrogen.

In some embodiments of Formula (I-A2), T¹ is selected from the group consisting of: —C(═O)N(R^{s})R^{z}, —S(O)₁₋₂N(R^{s})R^{z}, and —S(═NR^{s})(═O)N(R^{s})R^{z}. In some embodiments, T¹ is —C(═O)N(R^{s})R^{z}. For example, T¹ can be —C(═O)NR^{z}. In some embodiments, T¹ is —S(O)₂N(R^{s})R^{z}. For example, T¹ can be —S(O)₂NR^{z}. In some embodiments, T¹ is —S(═NR^{s})(═O)N(R^{s})R^{z}. For example, T¹ can be —S(═NH)(═O)N(R^{s})R^{z} (e.g., —S(═NH)(═O)N(H)R^{z}). In some embodiments, R^{z} is hydrogen or (C₁-C₆)alkyl optionally substituted with 1-4 R^{v}. In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula (I-A2), T¹ is —N(R^{s})C(═O)N(R^{s})R^{z} or —N(R^{s})C(═O)OR^{z}. In some embodiments, T¹ is —NHC(═O)NR^{z}. In some embodiments, T¹ is —N(H)C(═O)OR^{z}. In some embodiments, R^{z} is hydrogen or (C₁-C₆)alkyl optionally substituted with 1-4 R^{v}. In some embodiments, R^{z} is hydrogen. In some embodiments, R^{z} is (C₁-C₆)alkyl optionally substituted with 1-4 R^{v}. For example, R^{z} can be (C₁-C₆)alkyl, such as methyl, ethyl, or isopropyl.

In some embodiments of Formula (I-A2), T¹ is —(CR^{x}R^{x})_{q}—C(═O)OH; and q is 1, 2, or 3. In some embodiments, q is 1. In some embodiments, q is 2. In some embodiments, each R^{x} is hydrogen. In some embodiments, one pair of R^{x} on the same carbon, taken together with the carbon atom to which each is attached, forms a (C₃-C₈)cycloalkyl ring; and each remaining R^{x} when present is hydrogen. As a non-limiting example of the foregoing embodiments, R^{x} on the same carbon, taken together with the carbon atom to which each is attached, can form a cyclopropyl ring.

In some embodiments, T¹ is

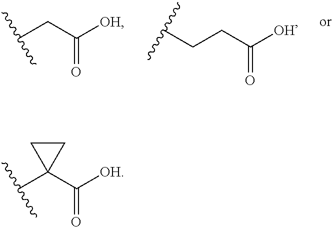 [SMILES: *CC(=O)O ; *CCC(=O)O ; *C1(C(=O)O)CC1]

In some embodiments of Formula (I-A2), T¹ is C(═O)OH.

In some embodiments of Formula (I-A2), T¹ is —O—(CR^{x}R^{x})_{q}—C(═O)OH; and q is 1, 2, or 3. In some embodiments, q is 1. In some embodiments, each R^{x} is hydrogen.

In some embodiments of Formula (I-A2), T¹ is —P(═O)R^{z1}R^{z2}. In some embodiments, R^{z1} and R^{z2} are independently selected (C₁-C₃)alkyl (e.g., methyl or ethyl).

In some embodiments of Formula (I-A2), T¹ is 5-membered heteroaryl having 2-4 ring heteroatoms selected from the group consisting of N, O, and S, wherein the heteroaryl is optionally substituted with 1-2 R^{v}. As non-limiting examples of the foregoing embodiments, T¹ can be selected from the group consisting of:

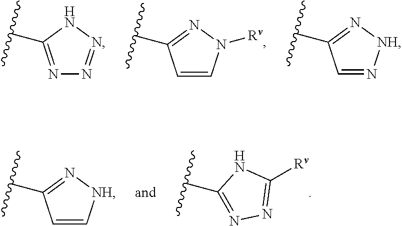

In some embodiments of Formula (I-A2), T¹ is 5- to 6-membered heteroaryl that comprises an endocyclic group selected from the group consisting of:

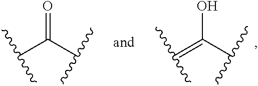

wherein the heteroaryl is further optionally substituted with 1-4 R^{v}. As non-limiting examples of the foregoing embodiments, T¹ can be selected from the group consisting of:

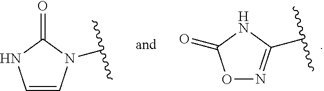 [SMILES: *n1cc[nH]c1=O ; *c1noc(=O)[nH]1]

In some embodiments of Formula (I-A2), T¹ is

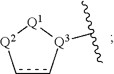

Q¹ is C(═O) or S(O)₂; Q² is O, NH, —CH₂—, or —CH₂—CH₂—; Q³ is N or CH; and [symbol] is a single bond or a double bond, provided that T⁴ is non-aromatic. As non-limiting examples of the foregoing embodiments, T¹ can be selected from the group consisting of:

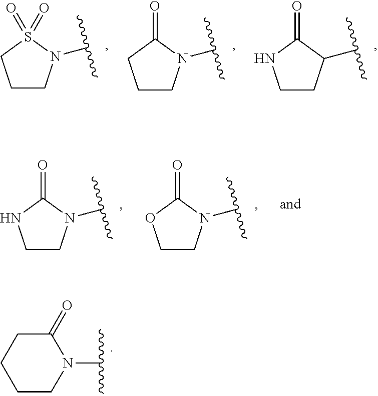

In some embodiments of Formula (I-A2), T¹ is (C₁-C₆)haloalkyl substituted with —OH and further optionally substituted with 1-2 R^{v}. In some embodiments, T¹ is (C₁-C₆)haloalkyl substituted with —OH. As a non-limiting example of the foregoing embodiments, T¹ can be

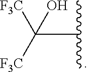 [SMILES: *C(O)(C(F)(F)F)C(F)(F)F]

In some embodiments of Formula (I-A2), T² is (C₁-C₆)alkyl which is substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

In some embodiments of Formula (I-A2), T² is (C₁-C₃)alkyl which is substituted with 4- to 6-membered heterocycloalkyl.

In some embodiments of Formula (I-A2), T² is (C₁-C₃)alkyl which is substituted with oxetanyl. As a non-limiting example of the foregoing embodiments, T² can be

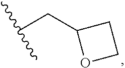 [SMILES: *CC1CCO1]

optionally wherein the stereogenic center in T² has (S)-configuration.

In some embodiments of Formula (I-A2), L¹ is CH₂; and Ring A is

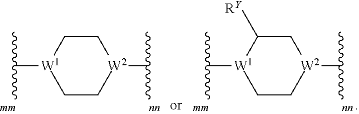

As a non-limiting example of the foregoing embodiments, Ring A can be

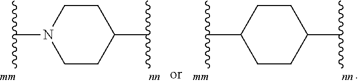 [SMILES: *C1CCN(*)CC1 ; *C1CCC(*)CC1]

In some embodiments of Formula (I-A2), L¹ is a bond; and Ring A is

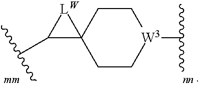

As a non-limiting example of the foregoing embodiments, Ring A can be

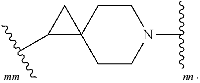 [SMILES: *C1CC12CCN(*)CC2]

In some embodiments of Formula (I-A2), L¹ is CH₂; and Ring A is

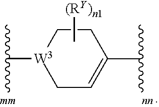

W³ is N or CH; and n1 is 0, 1, or 2. As a non-limiting example of the foregoing embodiments, Ring A can be

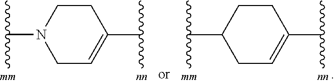 [SMILES: *C1=CCN(*)CC1 ; *C1=CCC(*)CC1]

In some embodiments of Formula (I-A2), Ring C is selected from the group consisting of: phenyl and 6-membered heteroaryl; and b is 1 or 2.

In some embodiments of Formula (I-A2),

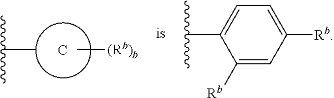

In some embodiments of Formula (I-A2),

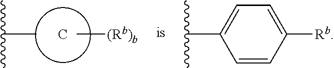

In some embodiments of Formula (I-A1), (I-A2), (I-A3), (I-A4), or (I-A5),

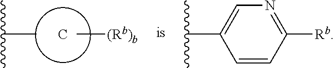

In some embodiments of Formula (I-A2), each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN.

In some embodiments of Formula (I-A2), each occurrence of R^{b} is independently selected from the group consisting of —F, —Cl, CF₃, and CN.

In some embodiments, the compound of Formula I is selected from the group consisting of the compounds in Table C1 or Table C2, or a pharmaceutically acceptable salt or solvate thereof.

TABLE C1

| Compound | |
|---|---|
| No. | Structure |
| 101 | 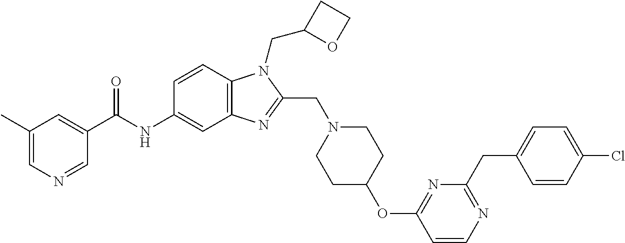 [SMILES: Cc1cncc(C(=O)Nc2ccc3c(c2)nc(CN2CCC(Oc4ccnc(Cc5ccc(Cl)cc5)n4)CC2)n3CC2CCO2)c1] |
| 102 | 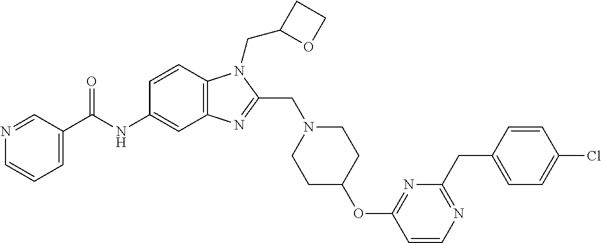 [SMILES: O=C(Nc1ccc2c(c1)nc(CN1CCC(Oc3ccnc(Cc4ccc(Cl)cc4)n3)CC1)n2CC1CCO1)c1cccnc1] |
| 103 | 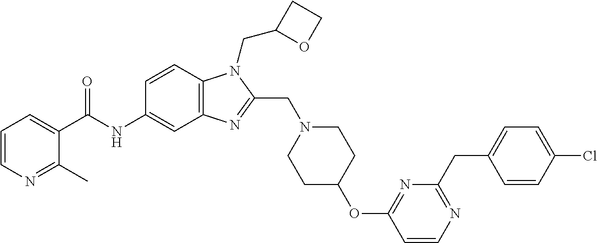 [SMILES: Cc1ncccc1C(=O)Nc1ccc2c(c1)nc(CN1CCC(Oc3ccnc(Cc4ccc(Cl)cc4)n3)CC1)n2CC1CCO1] |
| 104 | 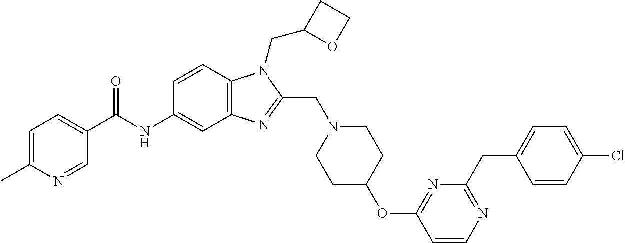 [SMILES: Cc1ccc(C(=O)Nc2ccc3c(c2)nc(CN2CCC(Oc4ccnc(Cc5ccc(Cl)cc5)n4)CC2)n3CC2CCO2)cn1] |
| 105 | 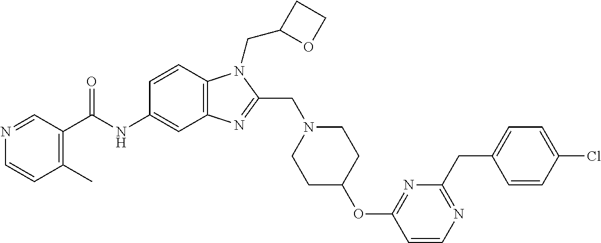 [SMILES: Cc1ccncc1C(=O)Nc1ccc2c(c1)nc(CN1CCC(Oc3ccnc(Cc4ccc(Cl)cc4)n3)CC1)n2CC1CCO1] |
| 106 | 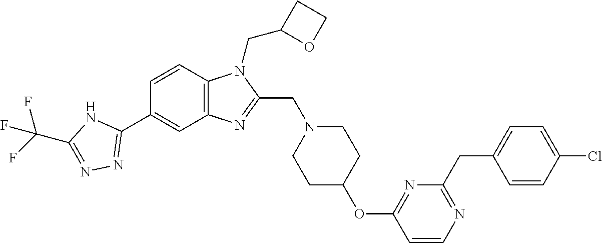 [SMILES: FC(F)(F)c1nnc(-c2ccc3c(c2)nc(CN2CCC(Oc4ccnc(Cc5ccc(Cl)cc5)n4)CC2)n3CC2CCO2)[nH]1] |
| 107 | 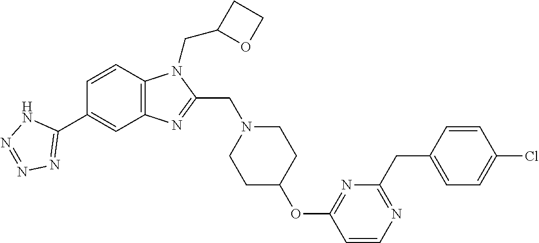 [SMILES: Clc1ccc(Cc2nccc(OC3CCN(Cc4nc5cc(-c6nnn[nH]6)ccc5n4CC4CCO4)CC3)n2)cc1] |
| 108 | 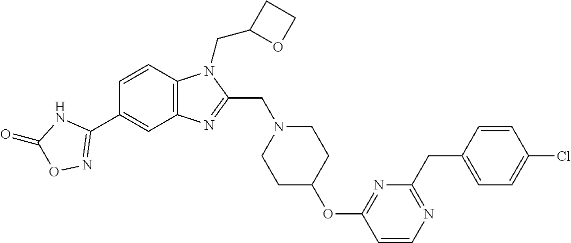 [SMILES: O=c1[nH]c(-c2ccc3c(c2)nc(CN2CCC(Oc4ccnc(Cc5ccc(Cl)cc5)n4)CC2)n3CC2CCO2)no1] |
| 109 | 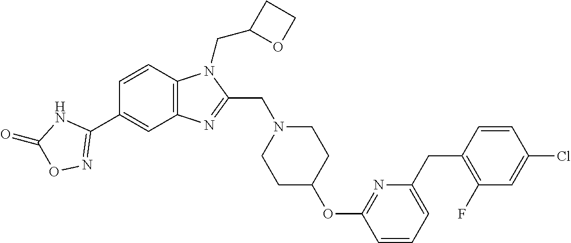 [SMILES: O=c1[nH]c(-c2ccc3c(c2)nc(CN2CCC(Oc4cccc(Cc5ccc(Cl)cc5F)n4)CC2)n3CC2CCO2)no1] |
| 110 | 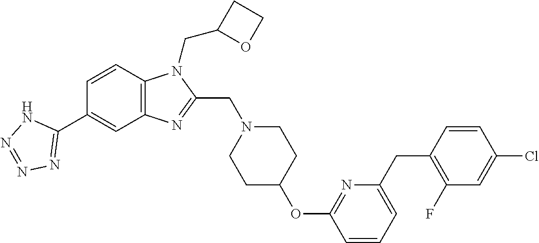 [SMILES: Fc1cc(Cl)ccc1Cc1cccc(OC2CCN(Cc3nc4cc(-c5nnn[nH]5)ccc4n3CC3CCO3)CC2)n1] |
| 111 | 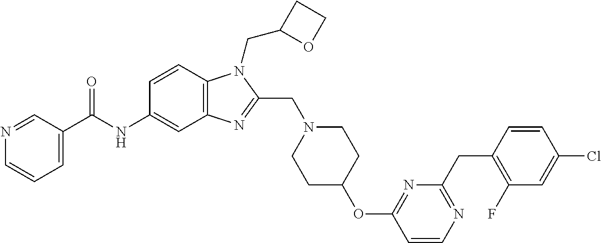 [SMILES: O=C(Nc1ccc2c(c1)nc(CN1CCC(Oc3ccnc(Cc4ccc(Cl)cc4F)n3)CC1)n2CC1CCO1)c1cccnc1] |
| 112 | 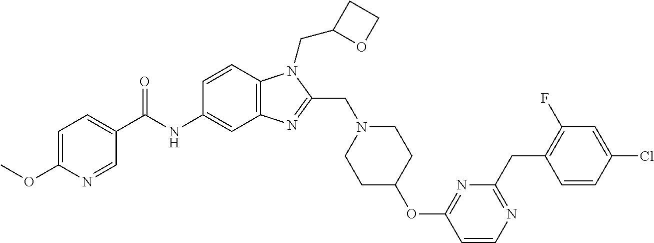 [SMILES: COc1ccc(C(=O)Nc2ccc3c(c2)nc(CN2CCC(Oc4ccnc(Cc5ccc(Cl)cc5F)n4)CC2)n3CC2CCO2)cn1] |
| 113 | 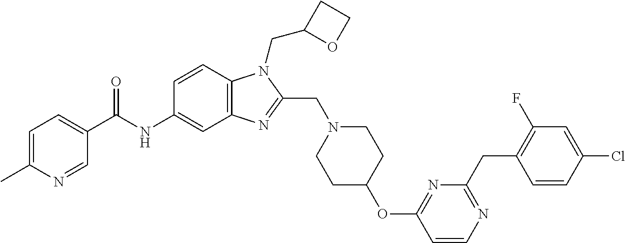 [SMILES: Cc1ccc(C(=O)Nc2ccc3c(c2)nc(CN2CCC(Oc4ccnc(Cc5ccc(Cl)cc5F)n4)CC2)n3CC2CCO2)cn1] |
| 114 | 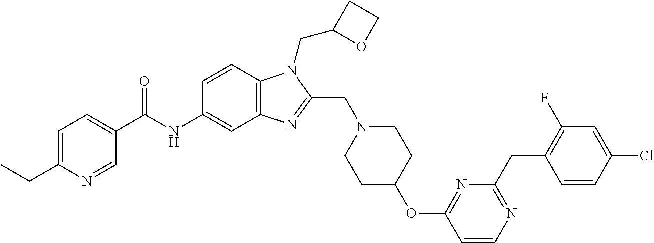 [SMILES: CCc1ccc(C(=O)Nc2ccc3c(c2)nc(CN2CCC(Oc4ccnc(Cc5ccc(Cl)cc5F)n4)CC2)n3CC2CCO2)cn1] |
| 115 | 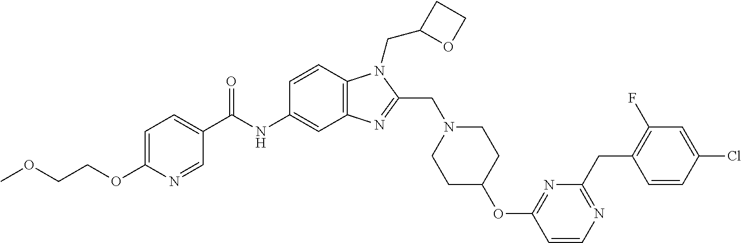 [SMILES: COCCOc1ccc(C(=O)Nc2ccc3c(c2)nc(CN2CCC(Oc4ccnc(Cc5ccc(Cl)cc5F)n4)CC2)n3CC2CCO2)cn1] |
| 116 | 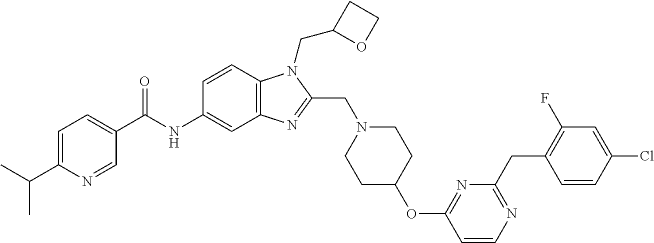 [SMILES: CC(C)c1ccc(C(=O)Nc2ccc3c(c2)nc(CN2CCC(Oc4ccnc(Cc5ccc(Cl)cc5F)n4)CC2)n3CC2CCO2)cn1] |
| 117 | 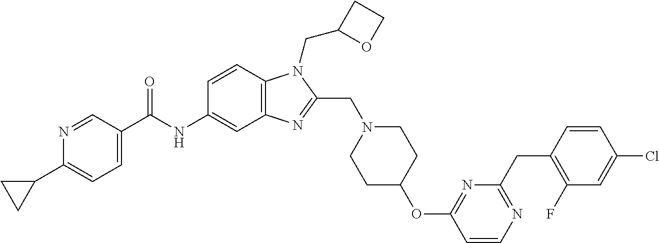 [SMILES: O=C(Nc1ccc2c(c1)nc(CN1CCC(Oc3ccnc(Cc4ccc(Cl)cc4F)n3)CC1)n2CC1CCO1)c1ccc(C2CC2)nc1] |
| 118 | 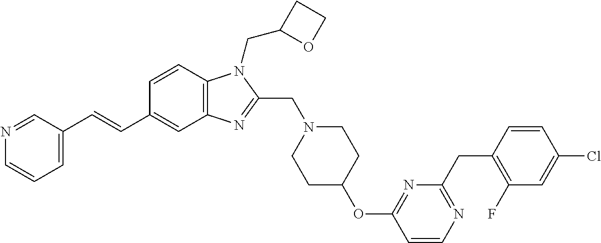 [SMILES: Fc1cc(Cl)ccc1Cc1nccc(OC2CCN(Cc3nc4cc(/C=C/c5cccnc5)ccc4n3CC3CCO3)CC2)n1] |
| 119 | 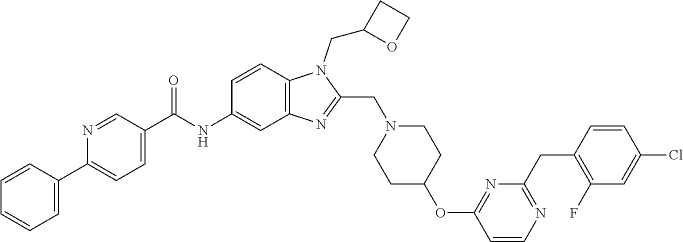 [SMILES: O=C(Nc1ccc2c(c1)nc(CN1CCC(Oc3ccnc(Cc4ccc(Cl)cc4F)n3)CC1)n2CC1CCO1)c1ccc(-c2ccccc2)nc1] |
| 120 | 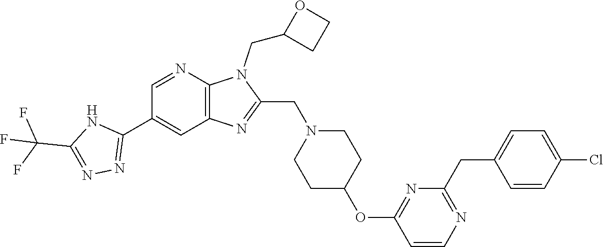 [SMILES: FC(F)(F)c1nnc(-c2cnc3c(c2)nc(CN2CCC(Oc4ccnc(Cc5ccc(Cl)cc5)n4)CC2)n3CC2CCO2)[nH]1] |
| 121 | 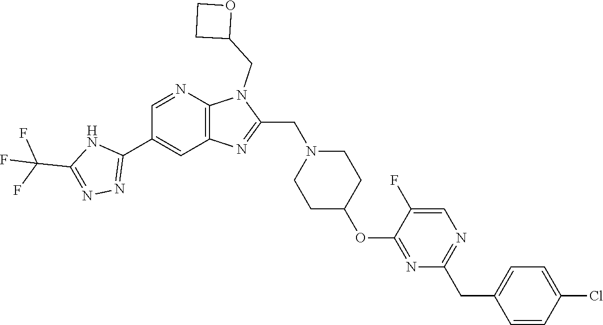 [SMILES: Fc1cnc(Cc2ccc(Cl)cc2)nc1OC1CCN(Cc2nc3cc(-c4nnc(C(F)(F)F)[nH]4)cnc3n2CC2CCO2)CC1] |
| 122 | 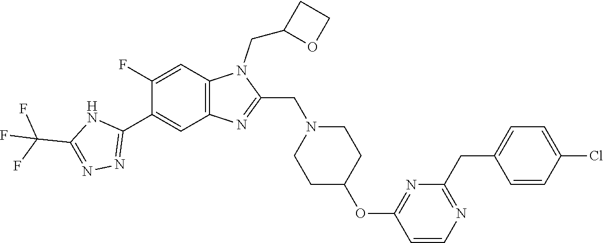 [SMILES: Fc1cc2c(cc1-c1nnc(C(F)(F)F)[nH]1)nc(CN1CCC(Oc3ccnc(Cc4ccc(Cl)cc4)n3)CC1)n2CC1CCO1] |
| 123 | 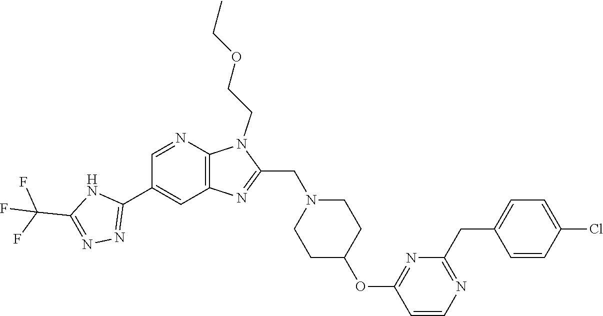 [SMILES: CCOCCn1c(CN2CCC(Oc3ccnc(Cc4ccc(Cl)cc4)n3)CC2)nc2cc(-c3nnc(C(F)(F)F)[nH]3)cnc21] |
| 124 | 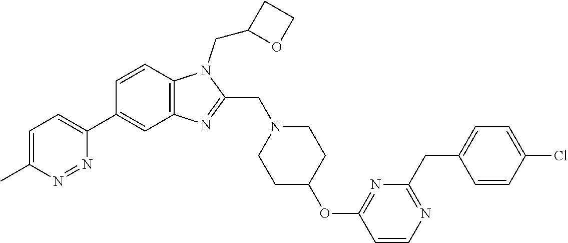 [SMILES: Cc1ccc(-c2ccc3c(c2)nc(CN2CCC(Oc4ccnc(Cc5ccc(Cl)cc5)n4)CC2)n3CC2CCO2)nn1] |
| 125 | 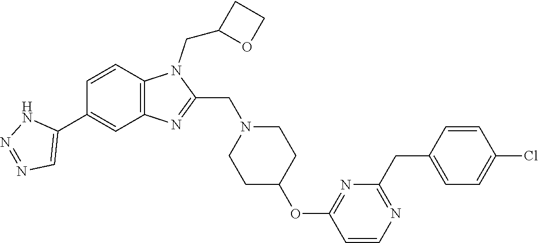 [SMILES: Clc1ccc(Cc2nccc(OC3CCN(Cc4nc5cc(-c6cnn[nH]6)ccc5n4CC4CCO4)CC3)n2)cc1] |
| 126 | 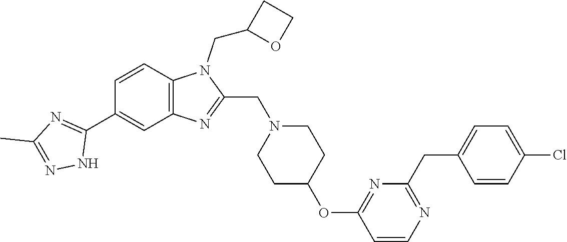 [SMILES: Cc1n[nH]c(-c2ccc3c(c2)nc(CN2CCC(Oc4ccnc(Cc5ccc(Cl)cc5)n4)CC2)n3CC2CCO2)n1] |
| 127 | 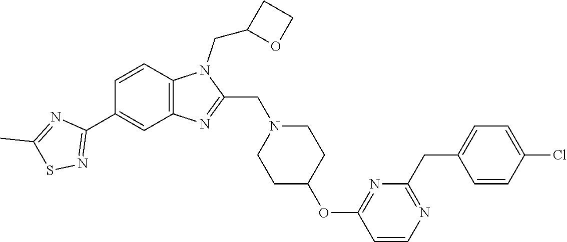 [SMILES: Cc1nc(-c2ccc3c(c2)nc(CN2CCC(Oc4ccnc(Cc5ccc(Cl)cc5)n4)CC2)n3CC2CCO2)ns1] |
| 128 | 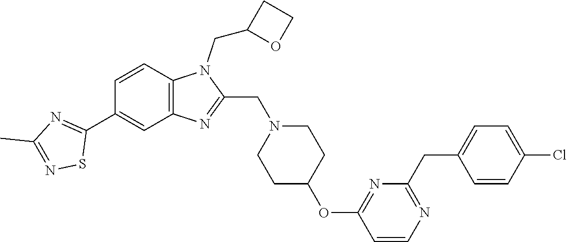 [SMILES: Cc1nsc(-c2ccc3c(c2)nc(CN2CCC(Oc4ccnc(Cc5ccc(Cl)cc5)n4)CC2)n3CC2CCO2)n1] |
| 129 | 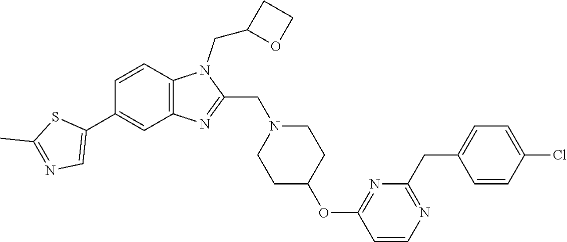 [SMILES: Cc1ncc(-c2ccc3c(c2)nc(CN2CCC(Oc4ccnc(Cc5ccc(Cl)cc5)n4)CC2)n3CC2CCO2)s1] |
| 130 | 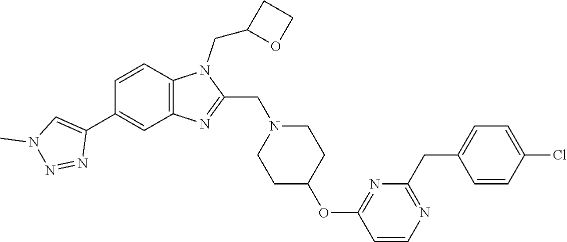 [SMILES: Cn1cc(-c2ccc3c(c2)nc(CN2CCC(Oc4ccnc(Cc5ccc(Cl)cc5)n4)CC2)n3CC2CCO2)nn1] |
| 131 | 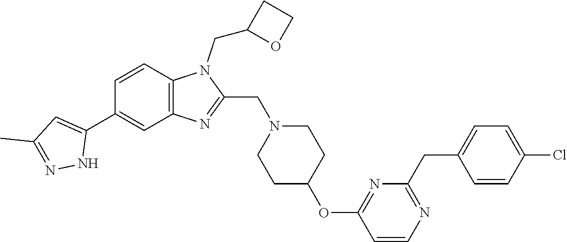 [SMILES: Cc1cc(-c2ccc3c(c2)nc(CN2CCC(Oc4ccnc(Cc5ccc(Cl)cc5)n4)CC2)n3CC2CCO2)[nH]n1] |
| 132 | 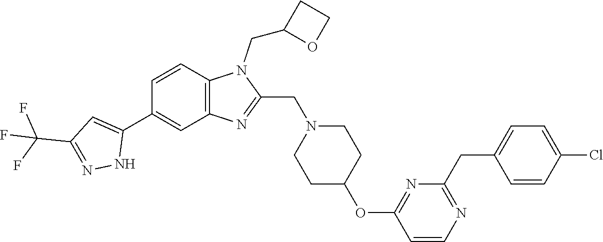 [SMILES: FC(F)(F)c1cc(-c2ccc3c(c2)nc(CN2CCC(Oc4ccnc(Cc5ccc(Cl)cc5)n4)CC2)n3CC2CCO2)[nH]n1] |
| 133 | 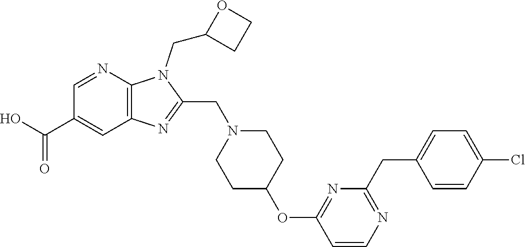 [SMILES: O=C(O)c1cnc2c(c1)nc(CN1CCC(Oc3ccnc(Cc4ccc(Cl)cc4)n3)CC1)n2CC1CCO1] |
| 134 | 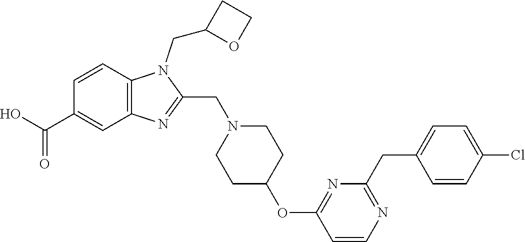 [SMILES: O=C(O)c1ccc2c(c1)nc(CN1CCC(Oc3ccnc(Cc4ccc(Cl)cc4)n3)CC1)n2CC1CCO1] |
| 135 | 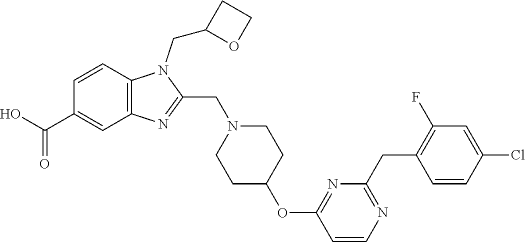 [SMILES: O=C(O)c1ccc2c(c1)nc(CN1CCC(Oc3ccnc(Cc4ccc(Cl)cc4F)n3)CC1)n2CC1CCO1] |
| 136 | 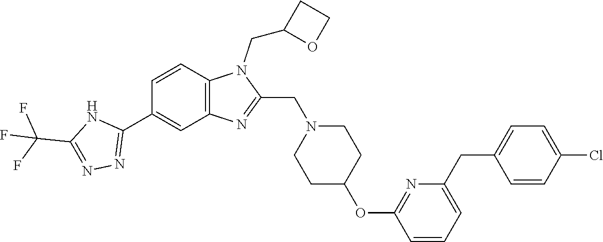 [SMILES: FC(F)(F)c1nnc(-c2ccc3c(c2)nc(CN2CCC(Oc4cccc(Cc5ccc(Cl)cc5)n4)CC2)n3CC2CCO2)[nH]1] |
| 137 | 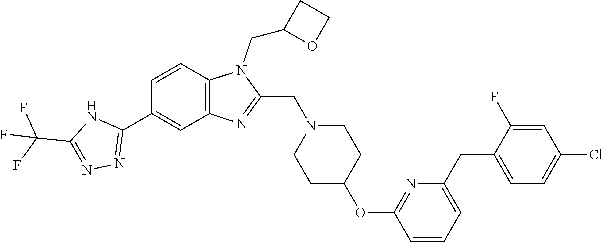 [SMILES: Fc1cc(Cl)ccc1Cc1cccc(OC2CCN(Cc3nc4cc(-c5nnc(C(F)(F)F)[nH]5)ccc4n3CC3CCO3)CC2)n1] |
| 138 | 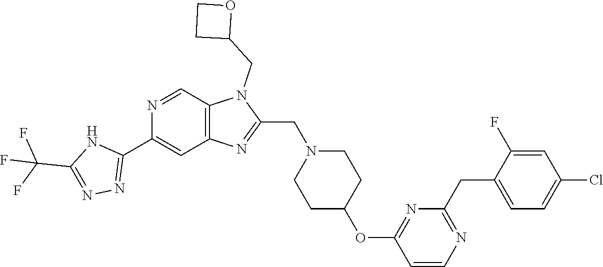 [SMILES: Fc1cc(Cl)ccc1Cc1nccc(OC2CCN(Cc3nc4cc(-c5nnc(C(F)(F)F)[nH]5)ncc4n3CC3CCO3)CC2)n1] |
| 139 | 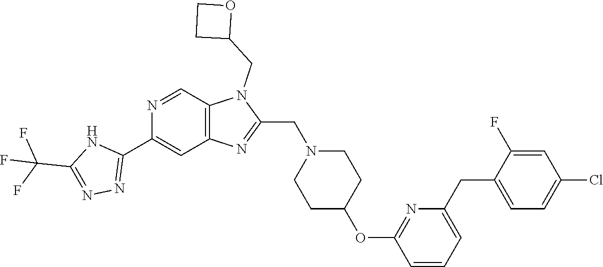 [SMILES: Fc1cc(Cl)ccc1Cc1cccc(OC2CCN(Cc3nc4cc(-c5nnc(C(F)(F)F)[nH]5)ncc4n3CC3CCO3)CC2)n1] |
| 140 | 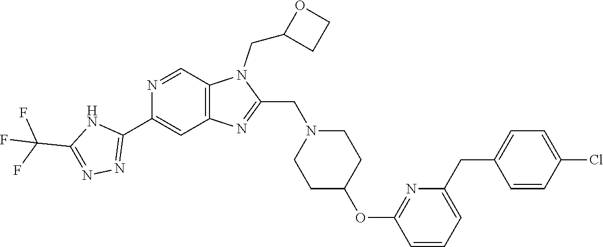 [SMILES: FC(F)(F)c1nnc(-c2cc3nc(CN4CCC(Oc5cccc(Cc6ccc(Cl)cc6)n5)CC4)n(CC4CCO4)c3cn2)[nH]1] |
| 141 | 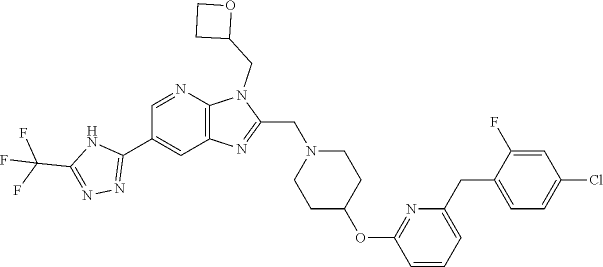 [SMILES: Fc1cc(Cl)ccc1Cc1cccc(OC2CCN(Cc3nc4cc(-c5nnc(C(F)(F)F)[nH]5)cnc4n3CC3CCO3)CC2)n1] |
| 142 | 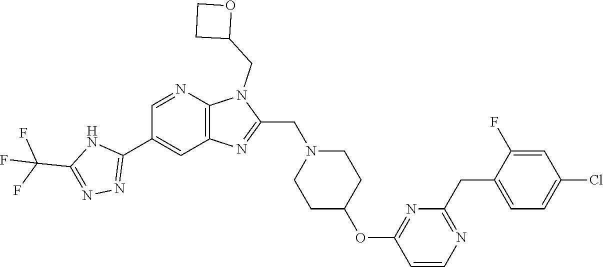 [SMILES: Fc1cc(Cl)ccc1Cc1nccc(OC2CCN(Cc3nc4cc(-c5nnc(C(F)(F)F)[nH]5)cnc4n3CC3CCO3)CC2)n1] |
| 143 | 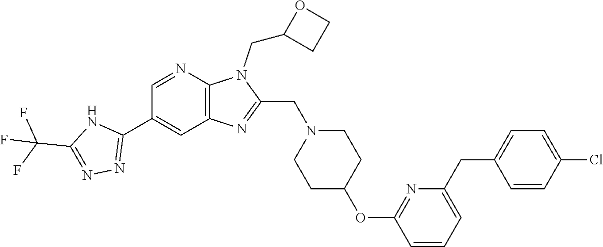 [SMILES: FC(F)(F)c1nnc(-c2cnc3c(c2)nc(CN2CCC(Oc4cccc(Cc5ccc(Cl)cc5)n4)CC2)n3CC2CCO2)[nH]1] |
| 144 | 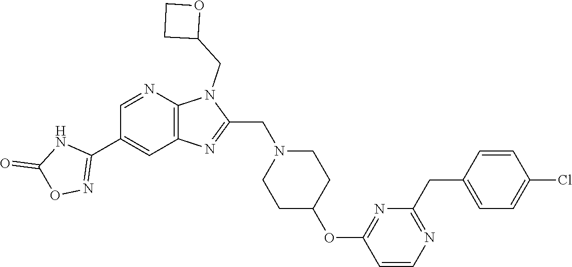 [SMILES: O=c1[nH]c(-c2cnc3c(c2)nc(CN2CCC(Oc4ccnc(Cc5ccc(Cl)cc5)n4)CC2)n3CC2CCO2)no1] |
| 145 | 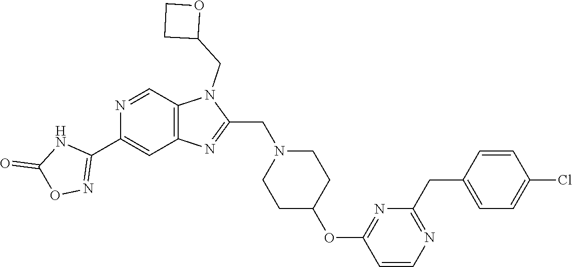 [SMILES: O=c1[nH]c(-c2cc3nc(CN4CCC(Oc5ccnc(Cc6ccc(Cl)cc6)n5)CC4)n(CC4CCO4)c3cn2)no1] |
| 146 | 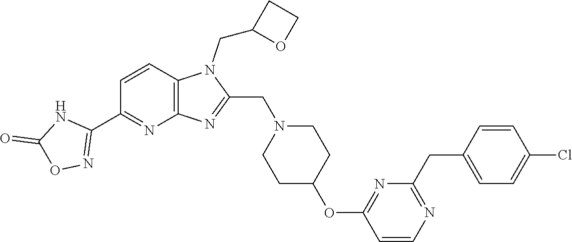 [SMILES: O=c1[nH]c(-c2ccc3c(n2)nc(CN2CCC(Oc4ccnc(Cc5ccc(Cl)cc5)n4)CC2)n3CC2CCO2)no1] |
| 147 | 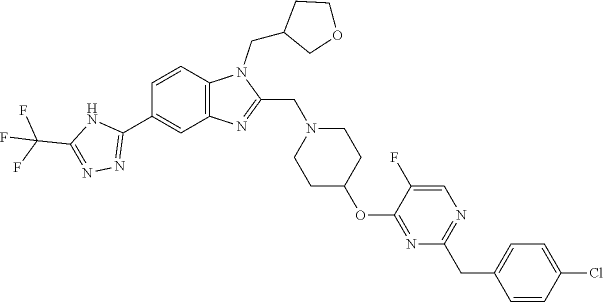 [SMILES: Fc1cnc(Cc2ccc(Cl)cc2)nc1OC1CCN(Cc2nc3cc(-c4nnc(C(F)(F)F)[nH]4)ccc3n2CC2CCOC2)CC1] |
| 148 | 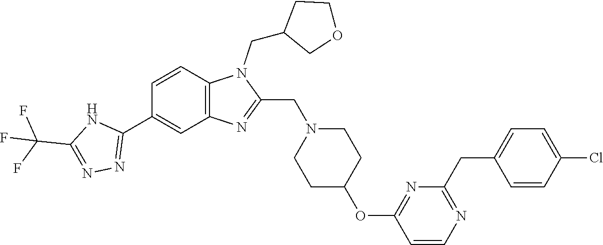 [SMILES: FC(F)(F)c1nnc(-c2ccc3c(c2)nc(CN2CCC(Oc4ccnc(Cc5ccc(Cl)cc5)n4)CC2)n3CC2CCOC2)[nH]1] |
| 149 | 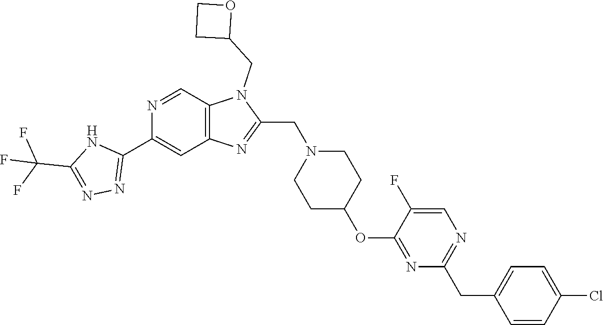 [SMILES: Fc1cnc(Cc2ccc(Cl)cc2)nc1OC1CCN(Cc2nc3cc(-c4nnc(C(F)(F)F)[nH]4)ncc3n2CC2CCO2)CC1] |
| 150 | 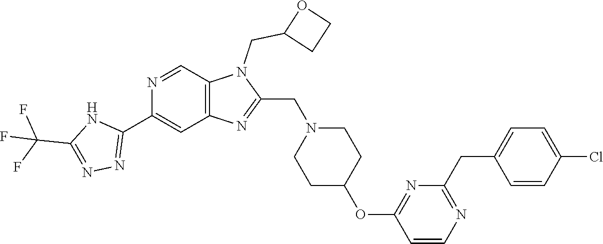 [SMILES: FC(F)(F)c1nnc(-c2cc3nc(CN4CCC(Oc5ccnc(Cc6ccc(Cl)cc6)n5)CC4)n(CC4CCO4)c3cn2)[nH]1] |
| 151 | 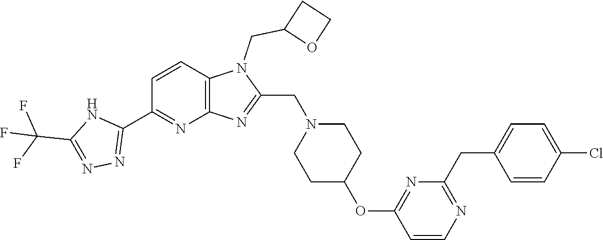 [SMILES: FC(F)(F)c1nnc(-c2ccc3c(n2)nc(CN2CCC(Oc4ccnc(Cc5ccc(Cl)cc5)n4)CC2)n3CC2CCO2)[nH]1] |
| 152 | 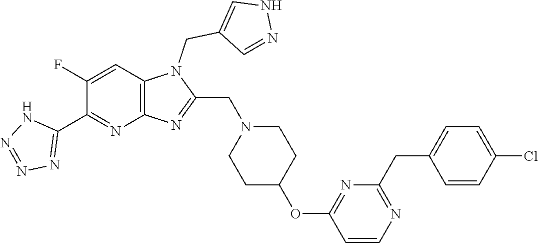 [SMILES: Fc1cc2c(nc1-c1nnn[nH]1)nc(CN1CCC(Oc3ccnc(Cc4ccc(Cl)cc4)n3)CC1)n2Cc1cn[nH]c1] |
| 153 | 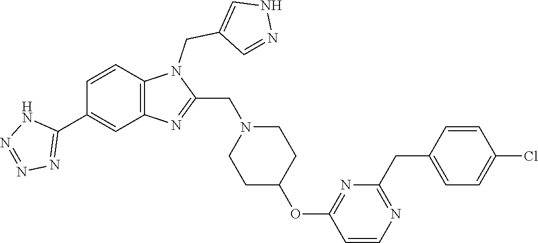 [SMILES: Clc1ccc(Cc2nccc(OC3CCN(Cc4nc5cc(-c6nnn[nH]6)ccc5n4Cc4cn[nH]c4)CC3)n2)cc1] |
| 154 | 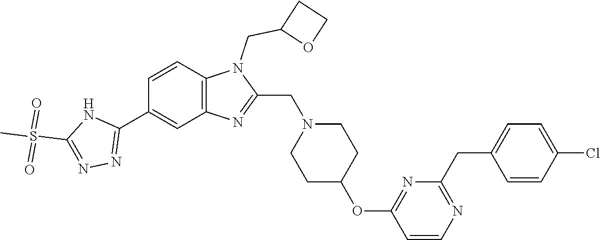 [SMILES: CS(=O)(=O)c1nnc(-c2ccc3c(c2)nc(CN2CCC(Oc4ccnc(Cc5ccc(Cl)cc5)n4)CC2)n3CC2CCO2)[nH]1] |
| 155 | 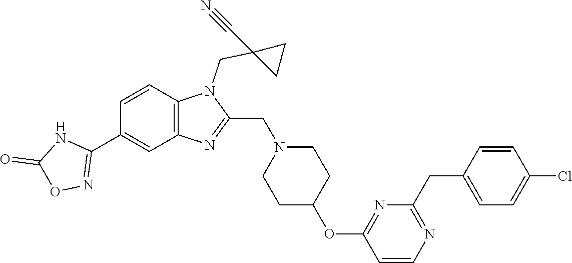 [SMILES: N#CC1(Cn2c(CN3CCC(Oc4ccnc(Cc5ccc(Cl)cc5)n4)CC3)nc3cc(-c4noc(=O)[nH]4)ccc32)CC1] |
| 156 | 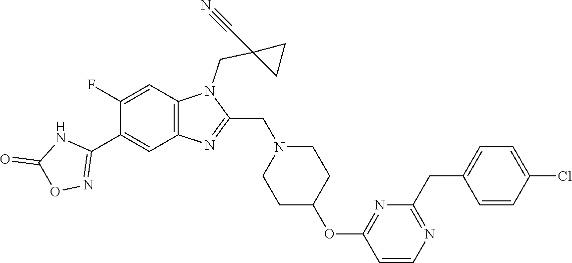 [SMILES: N#CC1(Cn2c(CN3CCC(Oc4ccnc(Cc5ccc(Cl)cc5)n4)CC3)nc3cc(-c4noc(=O)[nH]4)c(F)cc32)CC1] |
| 157 | 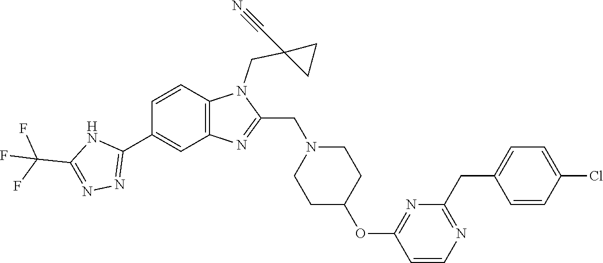 [SMILES: N#CC1(Cn2c(CN3CCC(Oc4ccnc(Cc5ccc(Cl)cc5)n4)CC3)nc3cc(-c4nnc(C(F)(F)F)[nH]4)ccc32)CC1] |
| 158 | 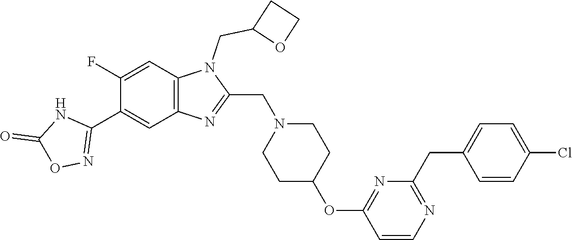 [SMILES: O=c1[nH]c(-c2cc3nc(CN4CCC(Oc5ccnc(Cc6ccc(Cl)cc6)n5)CC4)n(CC4CCO4)c3cc2F)no1] |
| 159 | 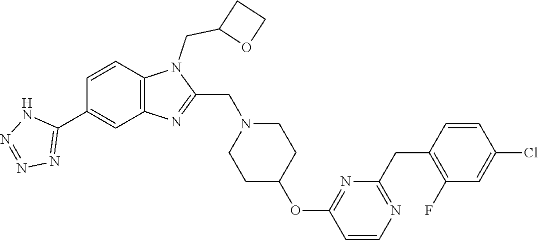 [SMILES: Fc1cc(Cl)ccc1Cc1nccc(OC2CCN(Cc3nc4cc(-c5nnn[nH]5)ccc4n3CC3CCO3)CC2)n1] |
| 160 | 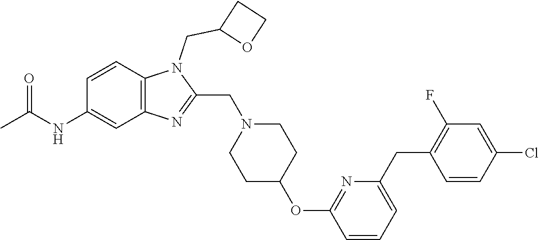 [SMILES: CC(=O)Nc1ccc2c(c1)nc(CN1CCC(Oc3cccc(Cc4ccc(Cl)cc4F)n3)CC1)n2CC1CCO1] |
| 161 | 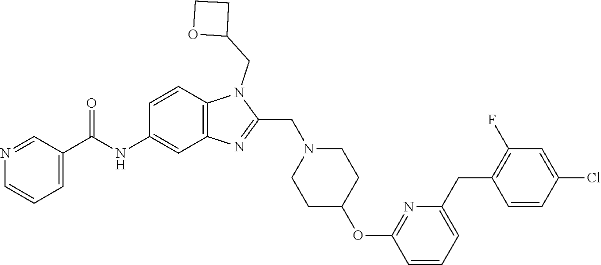 [SMILES: O=C(Nc1ccc2c(c1)nc(CN1CCC(Oc3cccc(Cc4ccc(Cl)cc4F)n3)CC1)n2CC1CCO1)c1cccnc1] |
| 162 | 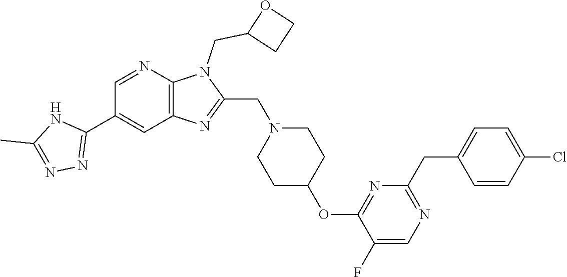 [SMILES: Cc1nnc(-c2cnc3c(c2)nc(CN2CCC(Oc4nc(Cc5ccc(Cl)cc5)ncc4F)CC2)n3CC2CCO2)[nH]1] |
| 163 | 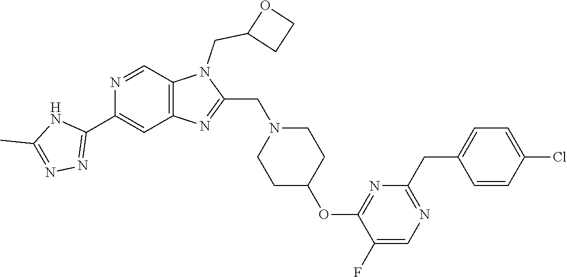 [SMILES: Cc1nnc(-c2cc3nc(CN4CCC(Oc5nc(Cc6ccc(Cl)cc6)ncc5F)CC4)n(CC4CCO4)c3cn2)[nH]1] |
| 164 | 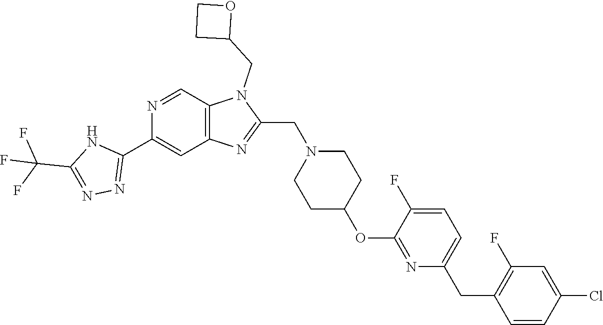 [SMILES: Fc1cc(Cl)ccc1Cc1ccc(F)c(OC2CCN(Cc3nc4cc(-c5nnc(C(F)(F)F)[nH]5)ncc4n3CC3CCO3)CC2)n1] |
| 165 | 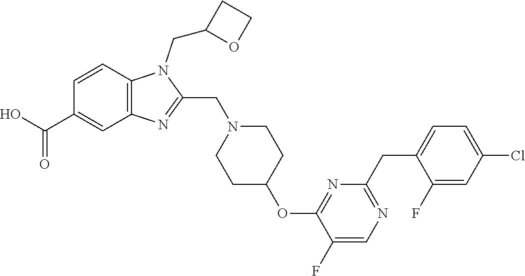 [SMILES: O=C(O)c1ccc2c(c1)nc(CN1CCC(Oc3nc(Cc4ccc(Cl)cc4F)ncc3F)CC1)n2CC1CCO1] |
| 166 | 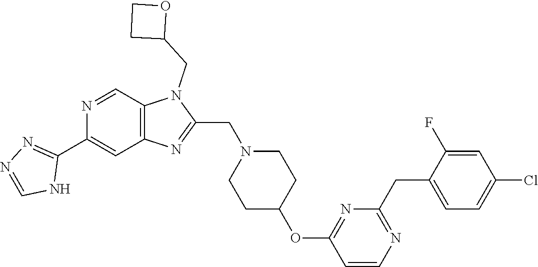 [SMILES: Fc1cc(Cl)ccc1Cc1nccc(OC2CCN(Cc3nc4cc(-c5nnc[nH]5)ncc4n3CC3CCO3)CC2)n1] |
| 167 | 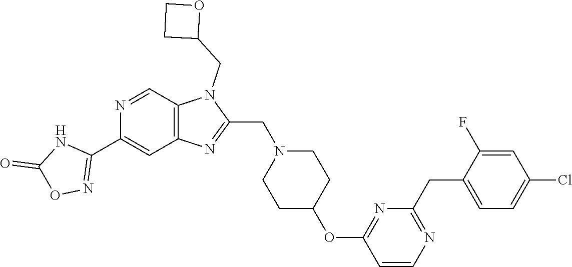 [SMILES: O=c1[nH]c(-c2cc3nc(CN4CCC(Oc5ccnc(Cc6ccc(Cl)cc6F)n5)CC4)n(CC4CCO4)c3cn2)no1] |
| 168 | 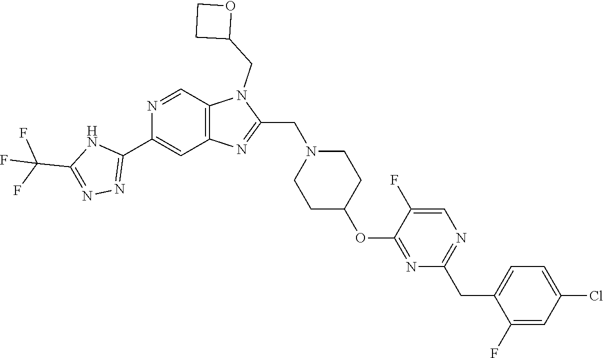 [SMILES: Fc1cc(Cl)ccc1Cc1ncc(F)c(OC2CCN(Cc3nc4cc(-c5nnc(C(F)(F)F)[nH]5)ncc4n3CC3CCO3)CC2)n1] |
| 169 | 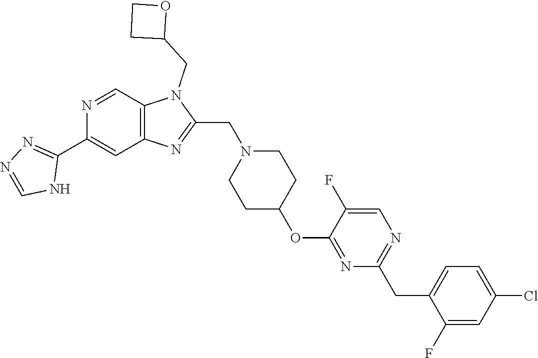 [SMILES: Fc1cc(Cl)ccc1Cc1ncc(F)c(OC2CCN(Cc3nc4cc(-c5nnc[nH]5)ncc4n3CC3CCO3)CC2)n1] |
| 170 | 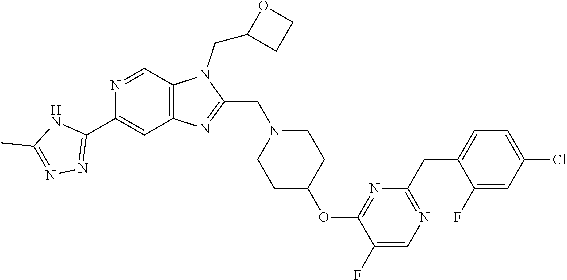 [SMILES: Cc1nnc(-c2cc3nc(CN4CCC(Oc5nc(Cc6ccc(Cl)cc6F)ncc5F)CC4)n(CC4CCO4)c3cn2)[nH]1] |
| 171 | 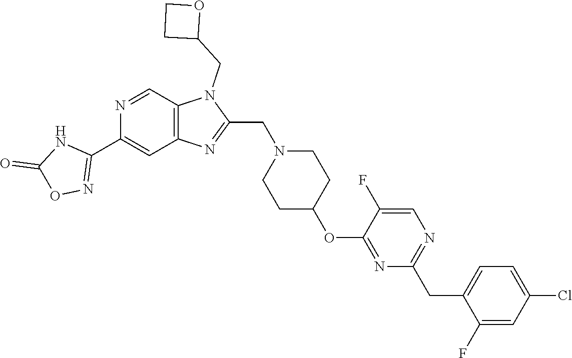 [SMILES: O=c1[nH]c(-c2cc3nc(CN4CCC(Oc5nc(Cc6ccc(Cl)cc6F)ncc5F)CC4)n(CC4CCO4)c3cn2)no1] |
| 172 | 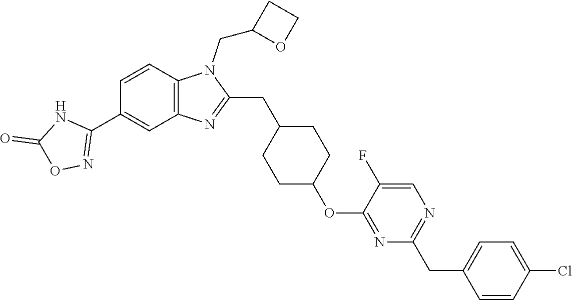 [SMILES: O=c1[nH]c(-c2ccc3c(c2)nc(CC2CCC(Oc4nc(Cc5ccc(Cl)cc5)ncc4F)CC2)n3CC2CCO2)no1] |
| 173 | 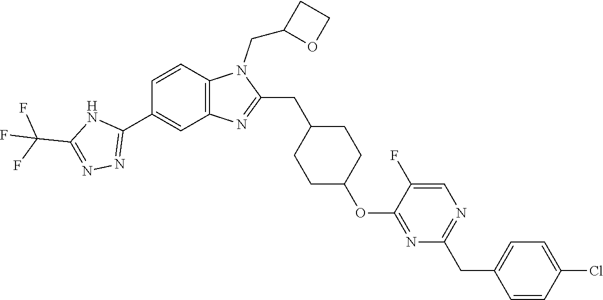 [SMILES: Fc1cnc(Cc2ccc(Cl)cc2)nc1OC1CCC(Cc2nc3cc(-c4nnc(C(F)(F)F)[nH]4)ccc3n2CC2CCO2)CC1] |
| 174 | 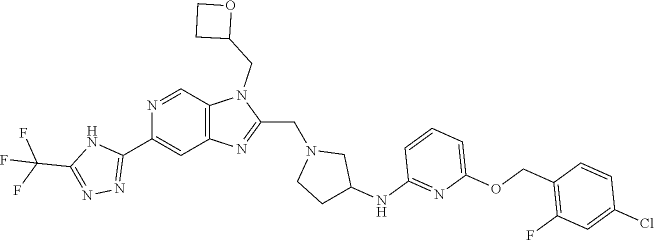 [SMILES: Fc1cc(Cl)ccc1COc1cccc(NC2CCN(Cc3nc4cc(-c5nnc(C(F)(F)F)[nH]5)ncc4n3CC3CCO3)C2)n1] |
| 175 | 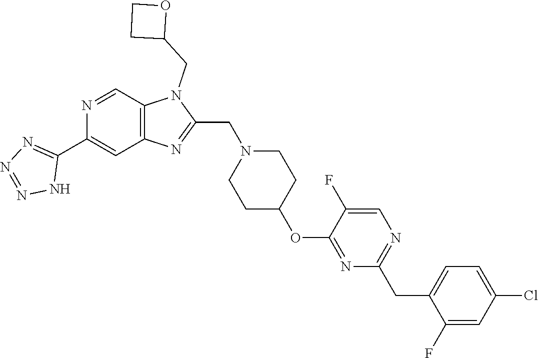 [SMILES: Fc1cc(Cl)ccc1Cc1ncc(F)c(OC2CCN(Cc3nc4cc(-c5nnn[nH]5)ncc4n3CC3CCO3)CC2)n1] |
| 176 | 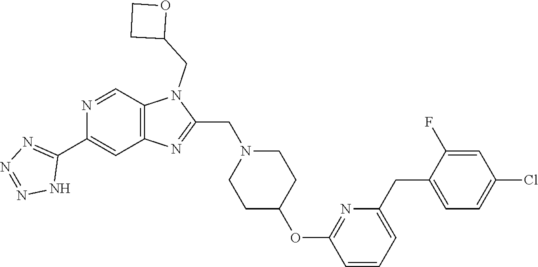 [SMILES: Fc1cc(Cl)ccc1Cc1cccc(OC2CCN(Cc3nc4cc(-c5nnn[nH]5)ncc4n3CC3CCO3)CC2)n1] |
| 177 | 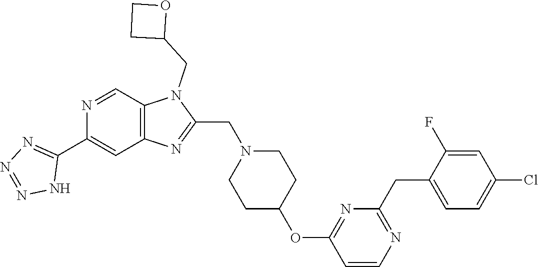 [SMILES: Fc1cc(Cl)ccc1Cc1nccc(OC2CCN(Cc3nc4cc(-c5nnn[nH]5)ncc4n3CC3CCO3)CC2)n1] |
| 178 | 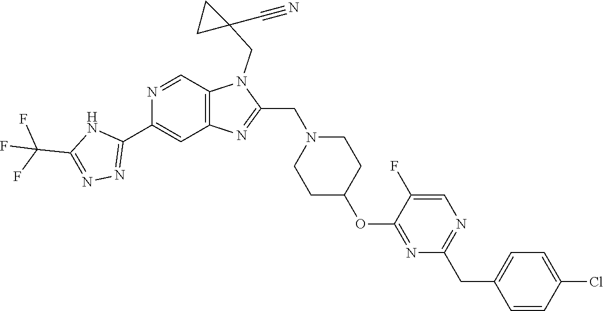 [SMILES: N#CC1(Cn2c(CN3CCC(Oc4nc(Cc5ccc(Cl)cc5)ncc4F)CC3)nc3cc(-c4nnc(C(F)(F)F)[nH]4)ncc32)CC1] |
| 179 | 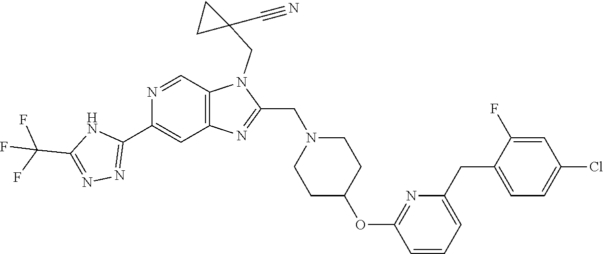 [SMILES: N#CC1(Cn2c(CN3CCC(Oc4cccc(Cc5ccc(Cl)cc5F)n4)CC3)nc3cc(-c4nnc(C(F)(F)F)[nH]4)ncc32)CC1] |
| 180 | 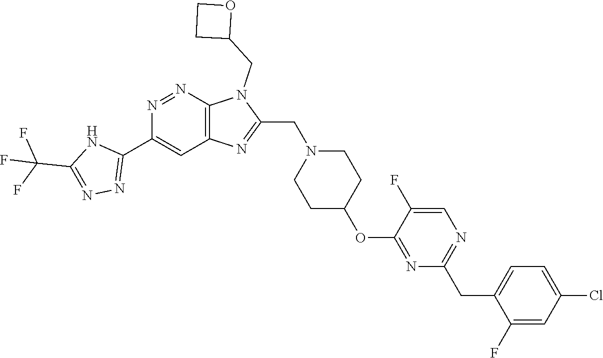 [SMILES: Fc1cc(Cl)ccc1Cc1ncc(F)c(OC2CCN(Cc3nc4cc(-c5nnc(C(F)(F)F)[nH]5)nnc4n3CC3CCO3)CC2)n1] |
| 181 | 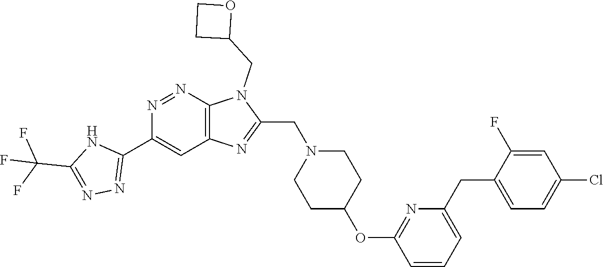 [SMILES: Fc1cc(Cl)ccc1Cc1cccc(OC2CCN(Cc3nc4cc(-c5nnc(C(F)(F)F)[nH]5)nnc4n3CC3CCO3)CC2)n1] |
| 182 | 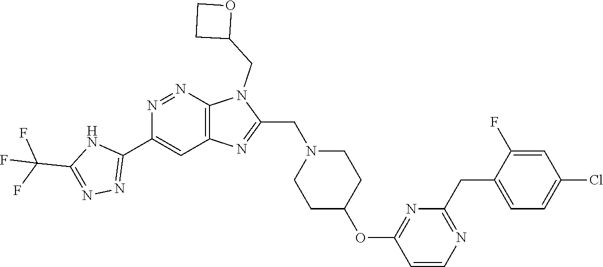 [SMILES: Fc1cc(Cl)ccc1Cc1nccc(OC2CCN(Cc3nc4cc(-c5nnc(C(F)(F)F)[nH]5)nnc4n3CC3CCO3)CC2)n1] |
| 183 | 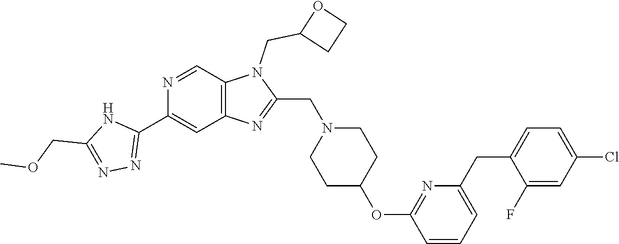 [SMILES: COCc1nnc(-c2cc3nc(CN4CCC(Oc5cccc(Cc6ccc(Cl)cc6F)n5)CC4)n(CC4CCO4)c3cn2)[nH]1] |
| 184 | 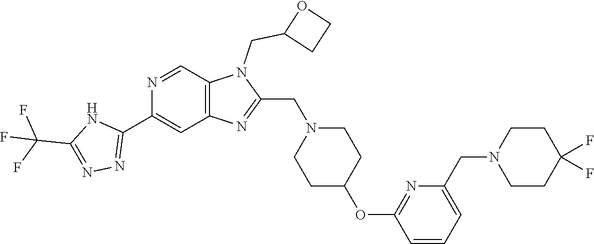 [SMILES: FC1(F)CCN(Cc2cccc(OC3CCN(Cc4nc5cc(-c6nnc(C(F)(F)F)[nH]6)ncc5n4CC4CCO4)CC3)n2)CC1] |
| 185 | 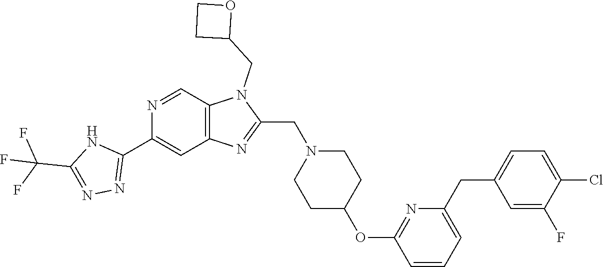 [SMILES: Fc1cc(Cc2cccc(OC3CCN(Cc4nc5cc(-c6nnc(C(F)(F)F)[nH]6)ncc5n4CC4CCO4)CC3)n2)ccc1Cl] |
| 186 | 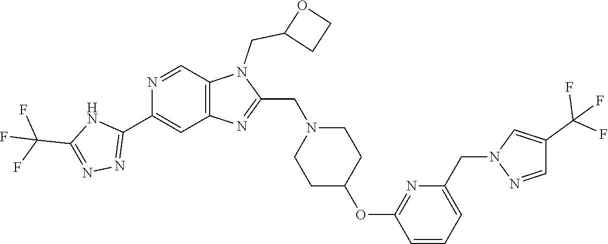 [SMILES: FC(F)(F)c1cnn(Cc2cccc(OC3CCN(Cc4nc5cc(-c6nnc(C(F)(F)F)[nH]6)ncc5n4CC4CCO4)CC3)n2)c1] |
| 187 | 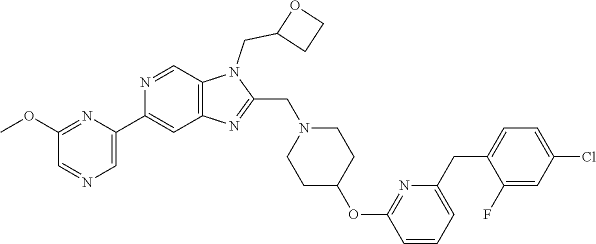 [SMILES: COc1cncc(-c2cc3nc(CN4CCC(Oc5cccc(Cc6ccc(Cl)cc6F)n5)CC4)n(CC4CCO4)c3cn2)n1] |
| 188 | 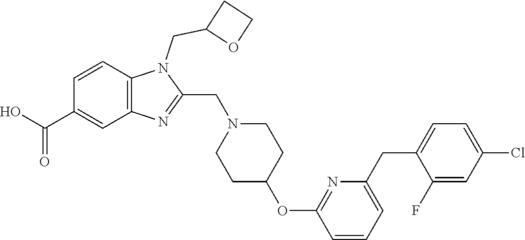 [SMILES: O=C(O)c1ccc2c(c1)nc(CN1CCC(Oc3cccc(Cc4ccc(Cl)cc4F)n3)CC1)n2CC1CCO1] |
| 189 | 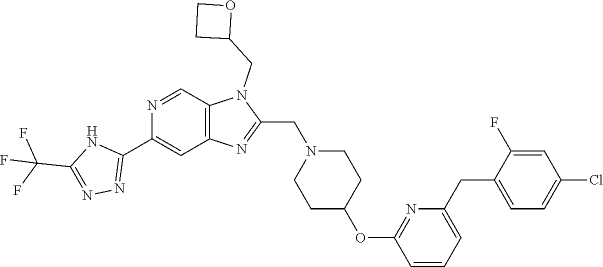 [SMILES: Fc1cc(Cl)ccc1Cc1cccc(OC2CCN(Cc3nc4cc(-c5nnc(C(F)(F)F)[nH]5)ncc4n3CC3CCO3)CC2)n1] |
| 190 | 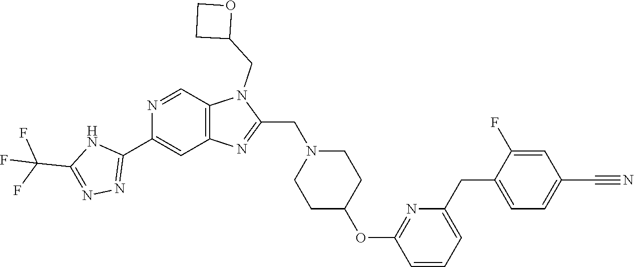 [SMILES: N#Cc1ccc(Cc2cccc(OC3CCN(Cc4nc5cc(-c6nnc(C(F)(F)F)[nH]6)ncc5n4CC4CCO4)CC3)n2)c(F)c1] |
| 191 | 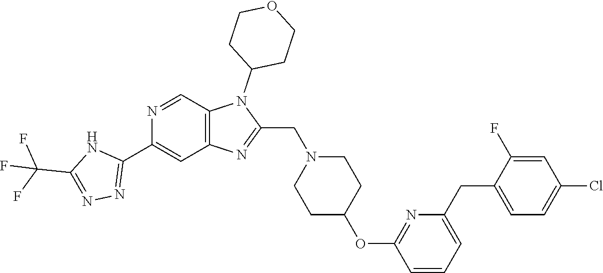 [SMILES: Fc1cc(Cl)ccc1Cc1cccc(OC2CCN(Cc3nc4cc(-c5nnc(C(F)(F)F)[nH]5)ncc4n3C3CCOCC3)CC2)n1] |
| 192 | 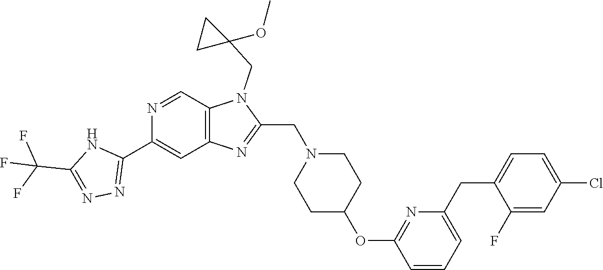 [SMILES: COC1(Cn2c(CN3CCC(Oc4cccc(Cc5ccc(Cl)cc5F)n4)CC3)nc3cc(-c4nnc(C(F)(F)F)[nH]4)ncc32)CC1] |
| 193 | 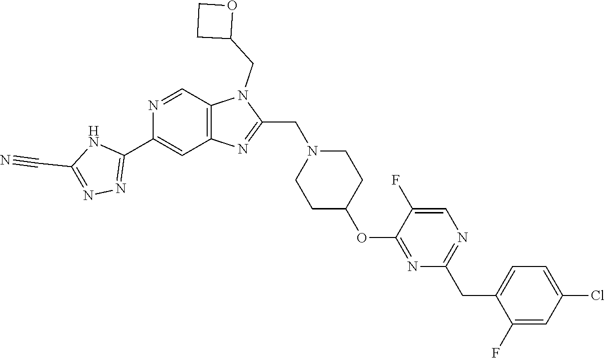 [SMILES: N#Cc1nnc(-c2cc3nc(CN4CCC(Oc5nc(Cc6ccc(Cl)cc6F)ncc5F)CC4)n(CC4CCO4)c3cn2)[nH]1] |

TABLE C2

| Compound | |
|---|---|
| No. | Structure |
| 101a | 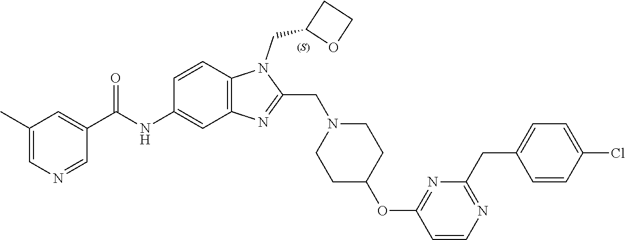 |
| 102a | 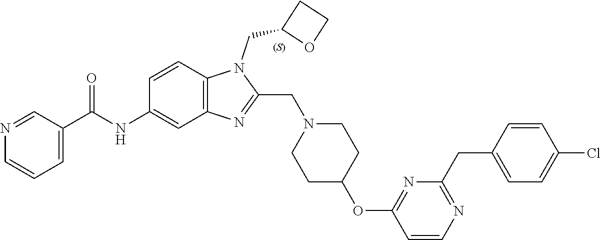 |
| 103a | 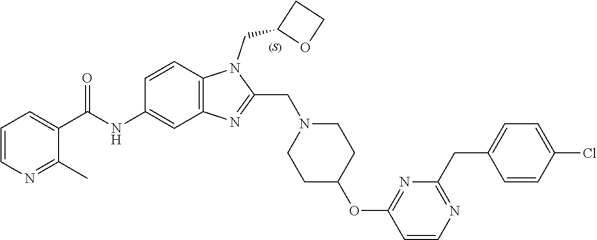 |
| 104a | 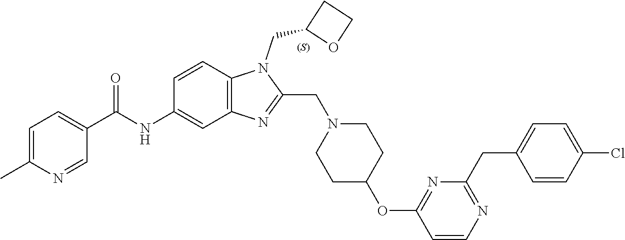 |
| 105a | 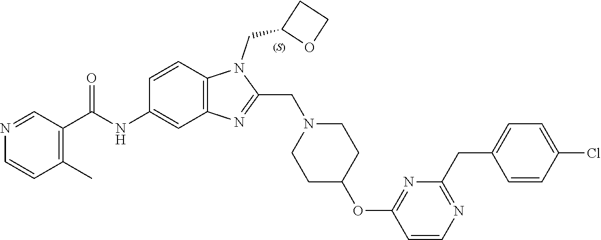 |
| 106a | 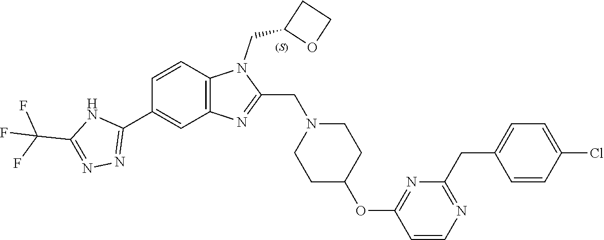 |
| 107a | 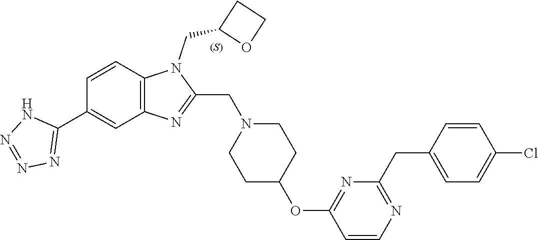 |
| 108a | 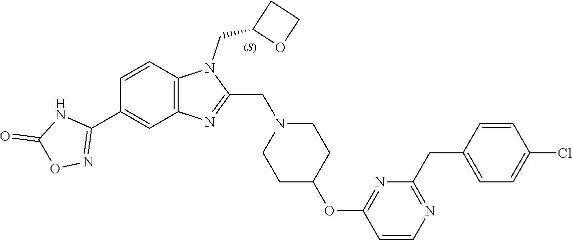 |
| 109a | 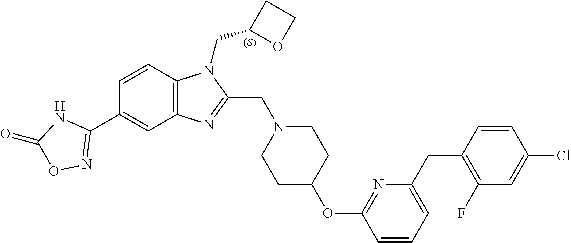 |
| 110a | 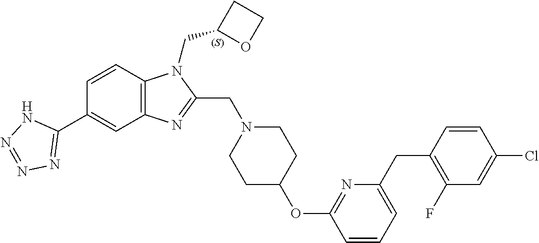 |
| 111a | 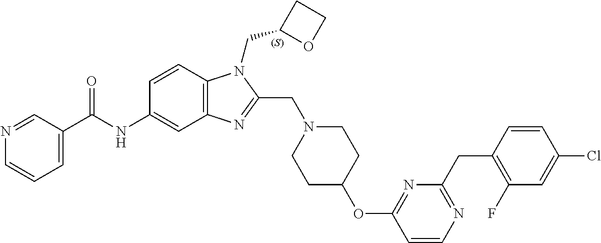 |
| 112a | 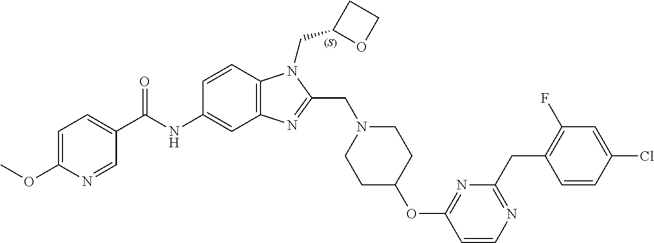 |
| 113a | 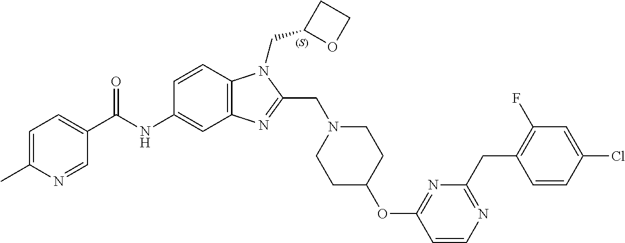 |
| 114a | 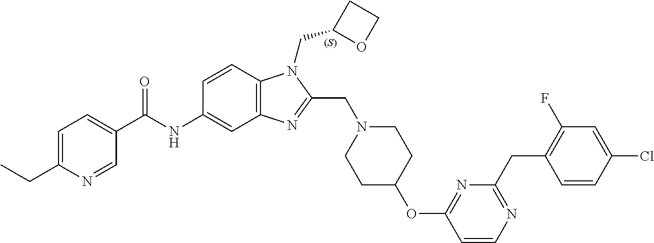 |
| 115a | 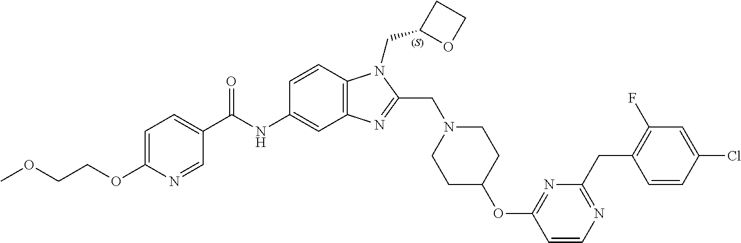 |
| 116a | 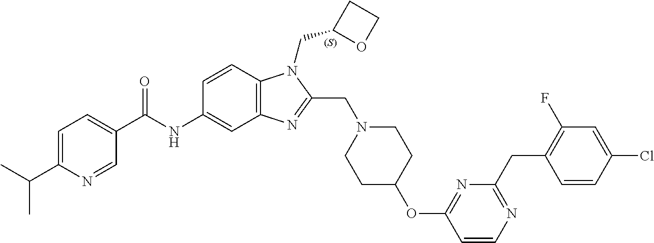 |
| 117a | 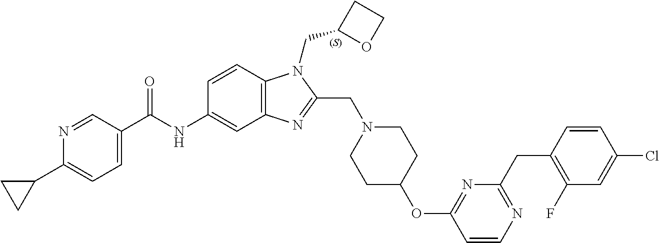 |
| 118a | 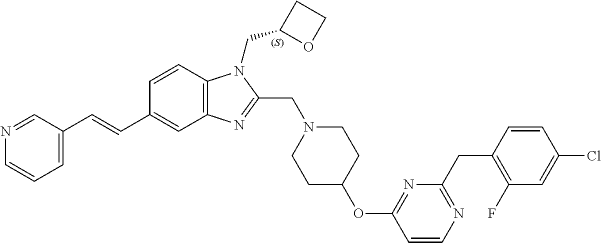 |
| 119a | 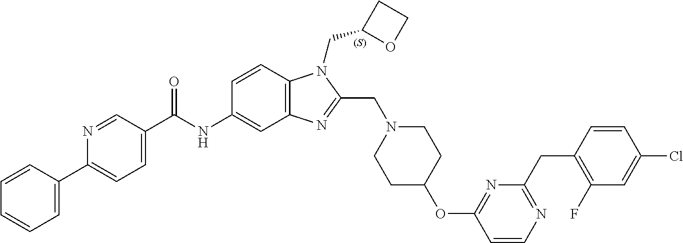 |
| 120a | 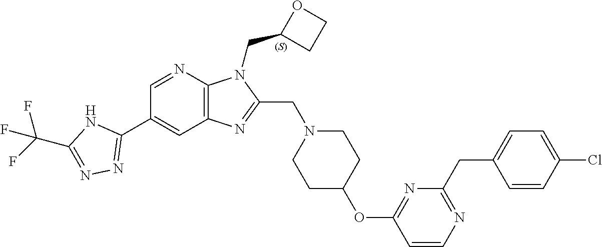 |
| 121a | 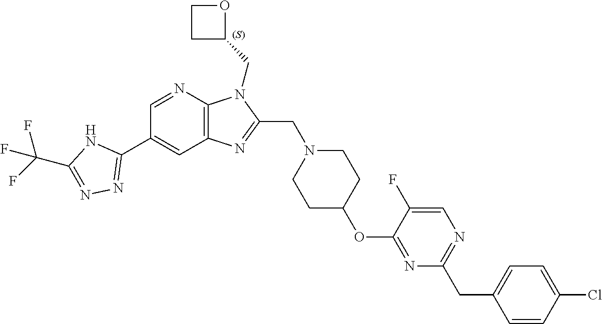 |
| 122a | 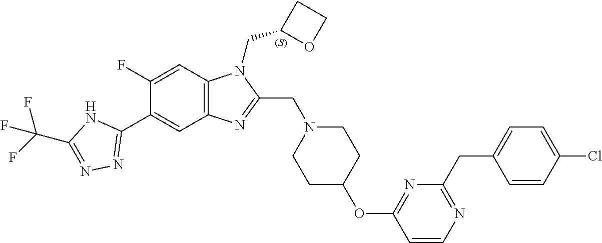 |
| 124a | 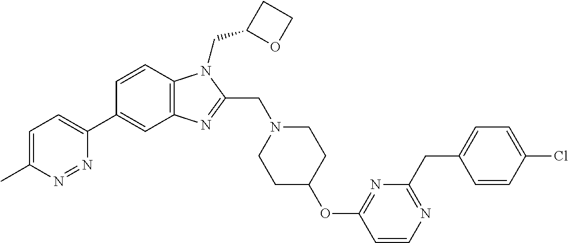 [SMILES: Cc1ccc(-c2ccc3c(c2)nc(CN2CCC(Oc4ccnc(Cc5ccc(Cl)cc5)n4)CC2)n3C[C@@H]2CCO2)nn1] |
| 125a | 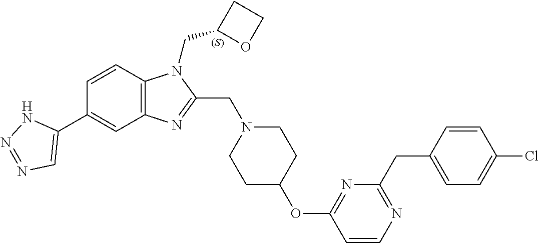 |
| 126a | 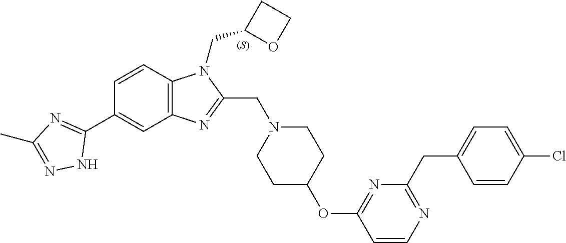 |
| 127a | 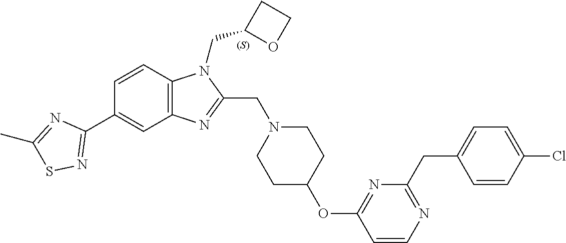 |
| 128a | 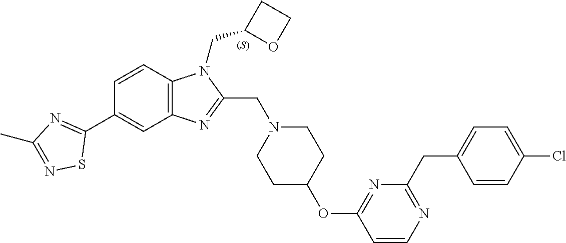 |
| 129a | 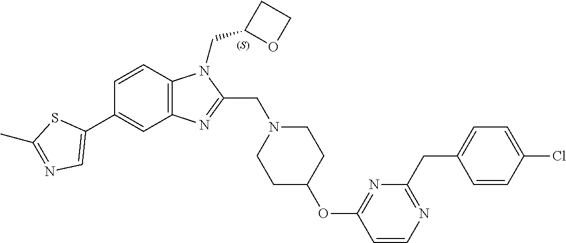 |
| 130a | 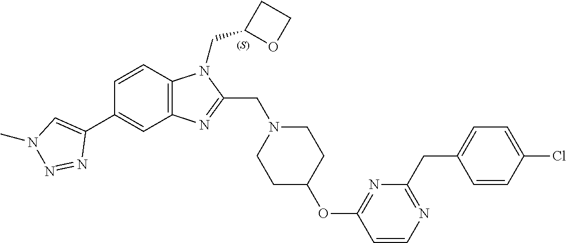 |
| 131a | 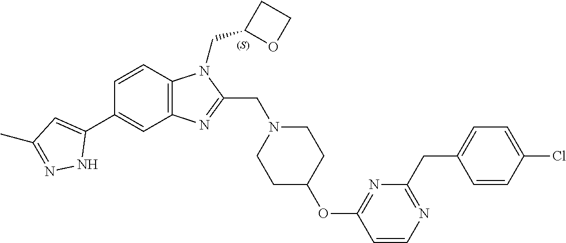 |
| 132a | 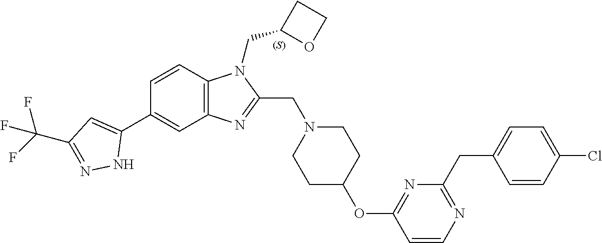 |
| 133a | 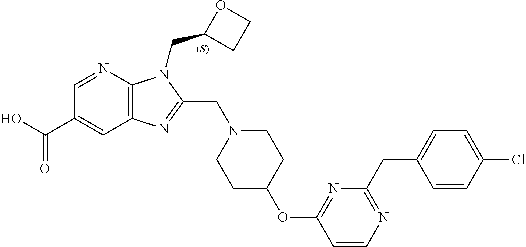 |
| 134a | 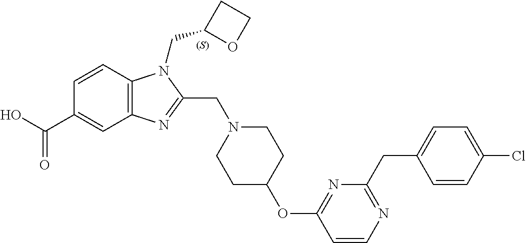 |
| 135a | 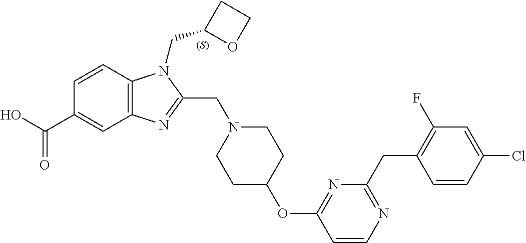 |
| 136a | 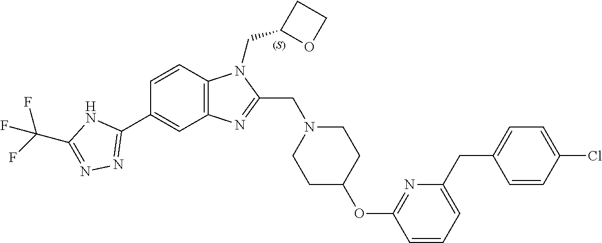 |
| 137a | 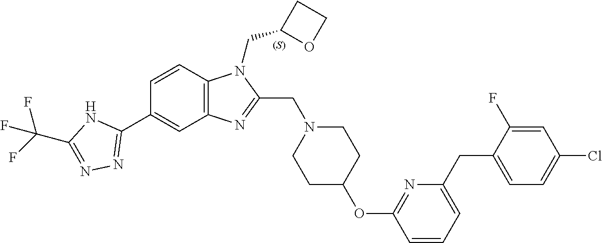 |
| 138a | 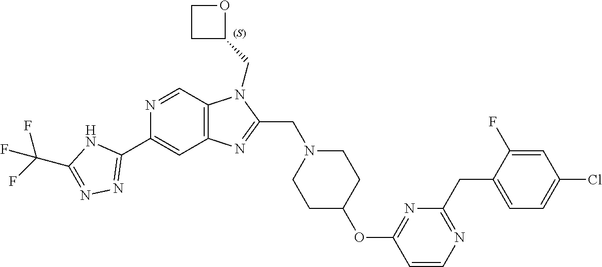 |
| 139a | 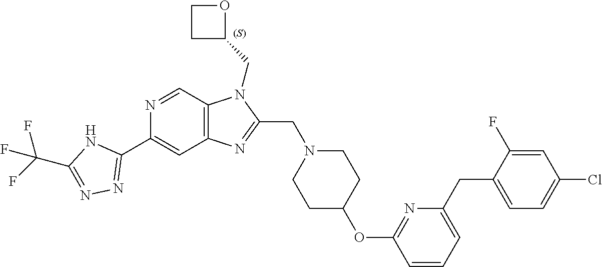 |
| 140a | 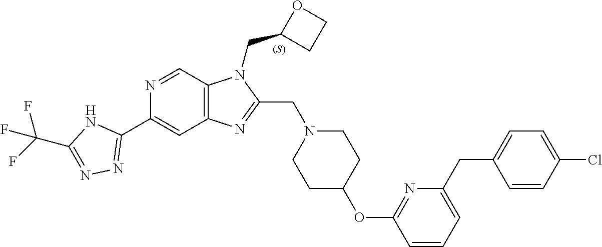 |
| 141a | 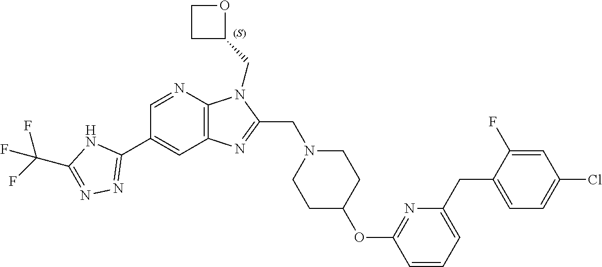 |
| 142a | 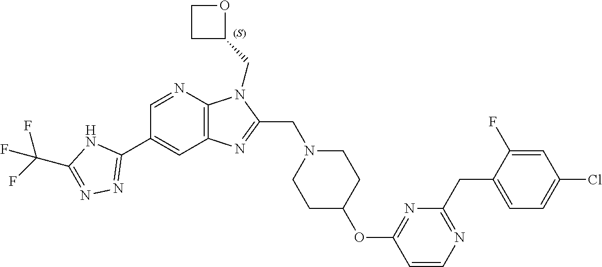 |
| 143a | 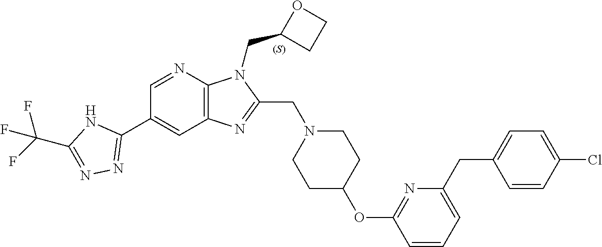 |
| 144a | 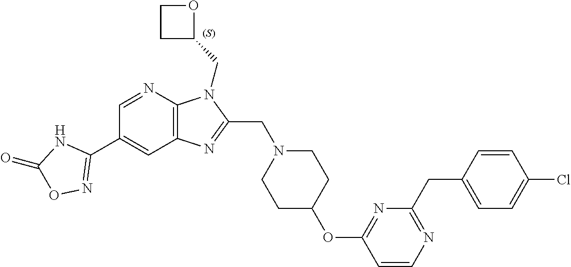 |
| 145a | 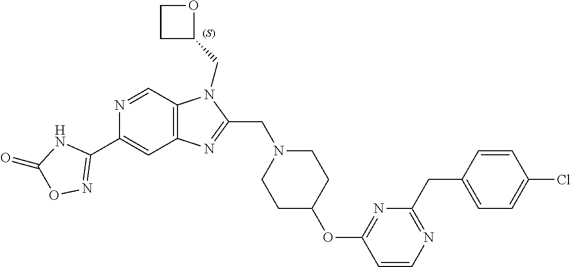 |
| 146a | 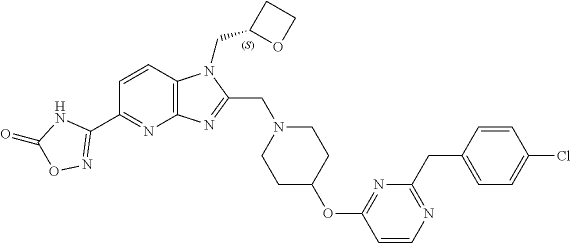 |
| 147a | 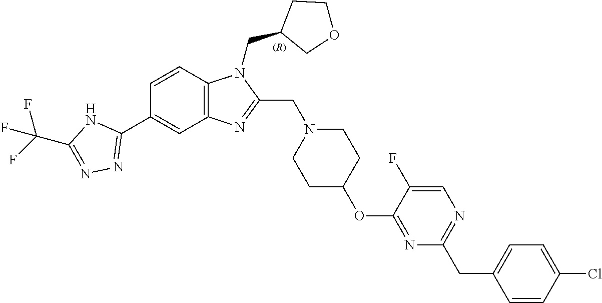 |
| 148a | 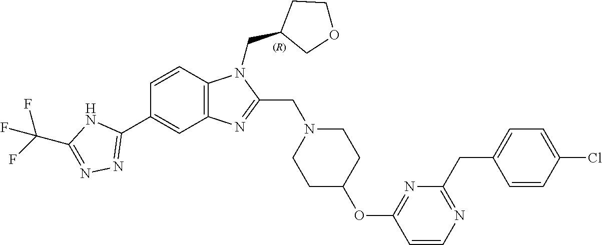 |
| 149a | 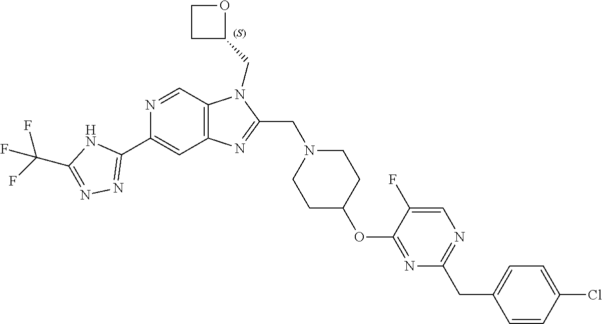 |
| 150a | 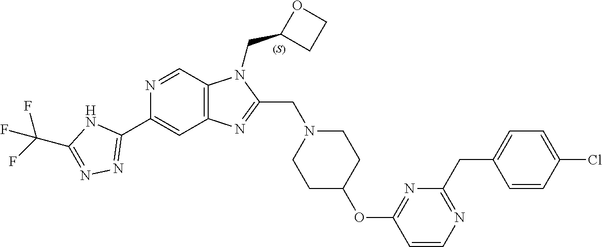 |
| 151a | 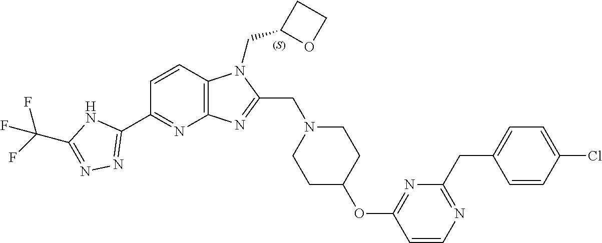 |
| 154a | 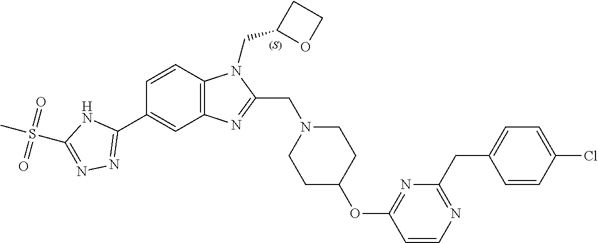 |
| 158a | 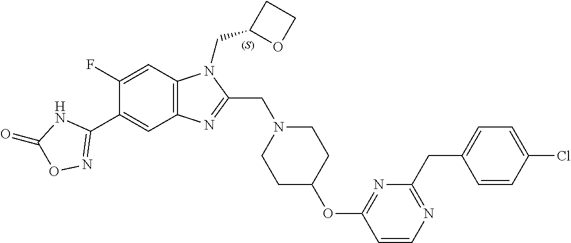 |
| 159a | 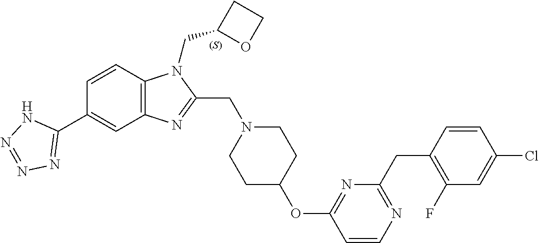 |
| 160a | 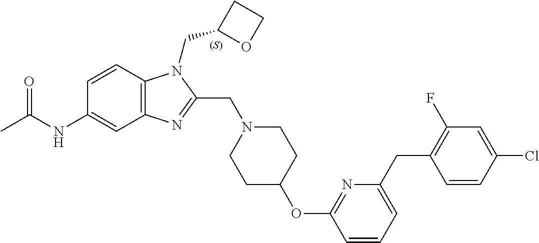 |
| 161a | 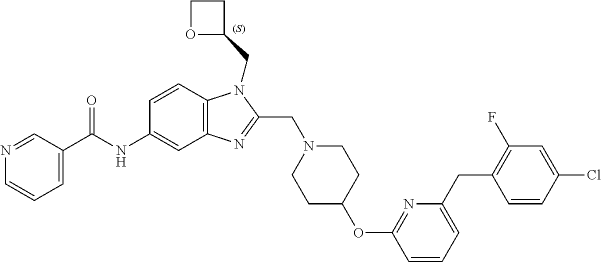 |
| 162a | 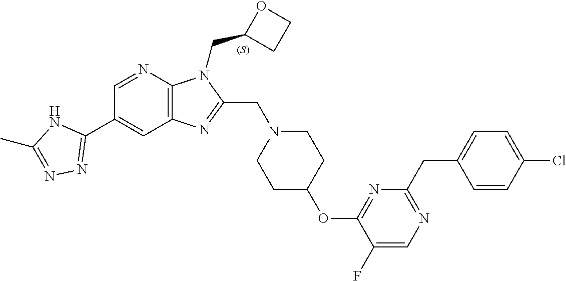 |
| 163a | 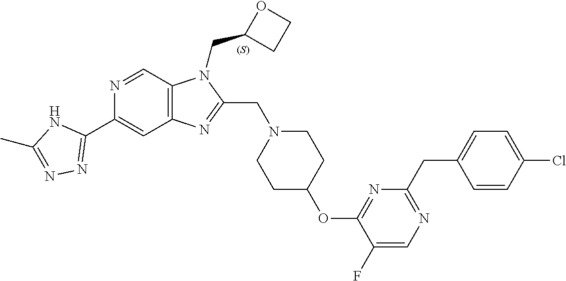 |
| 164a | 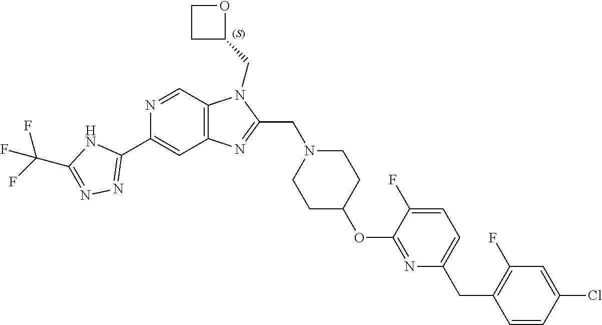 |
| 165a | 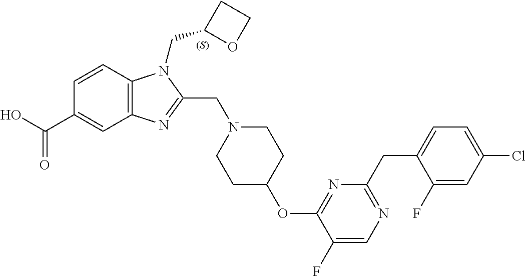 |
| 166a | 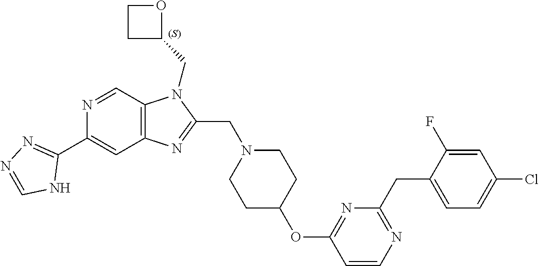 |
| 167a | 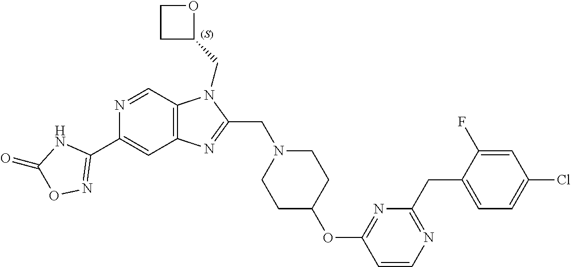 |
| 168a | 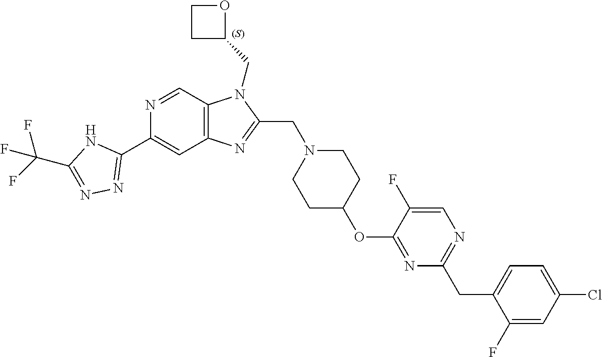 |
| 169a | 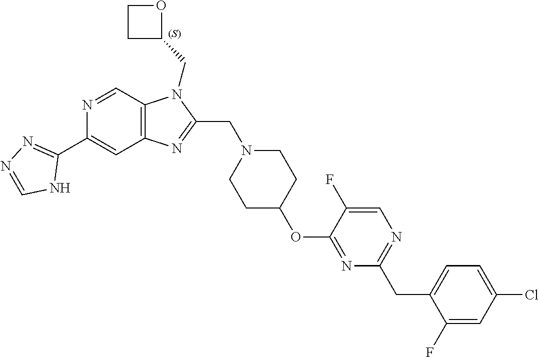 |
| 170a | 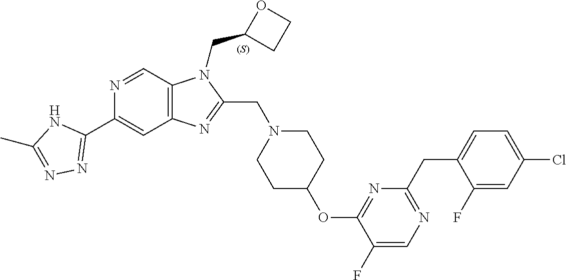 |
| 171a | 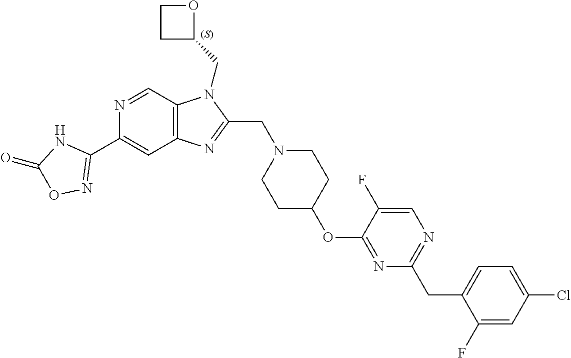 |
| 172a | 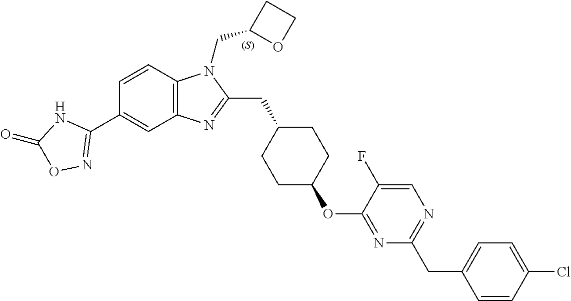 |
| 173a | 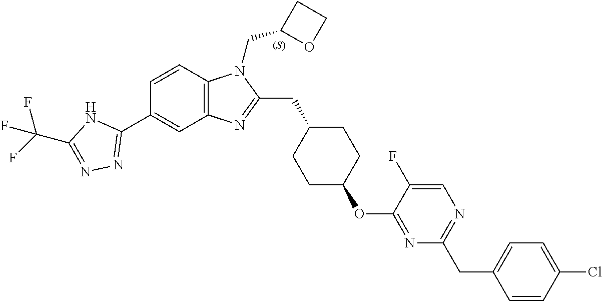 |
| 174a | 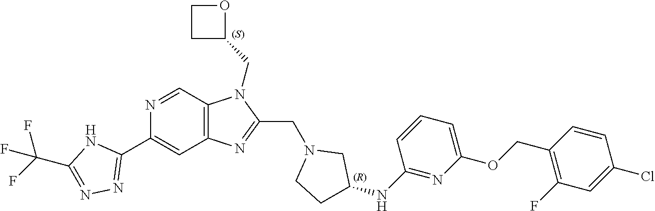 |
| 175a | 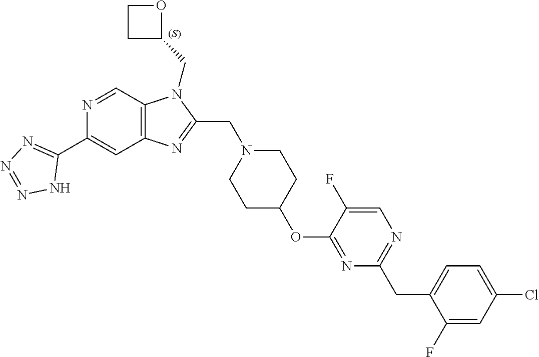 |
| 176a | 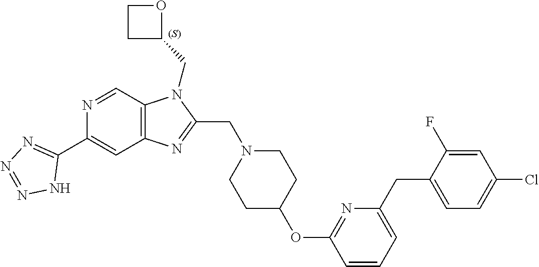 |
| 177a | 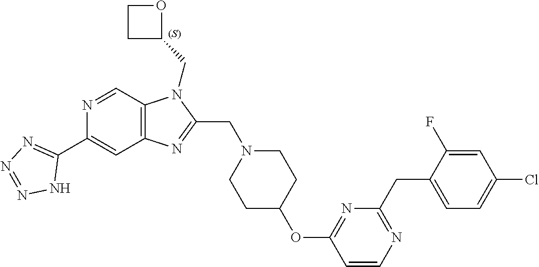 |
| 180a | 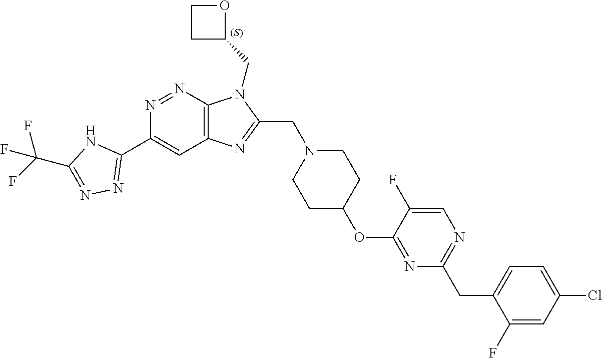 |
| 181a | 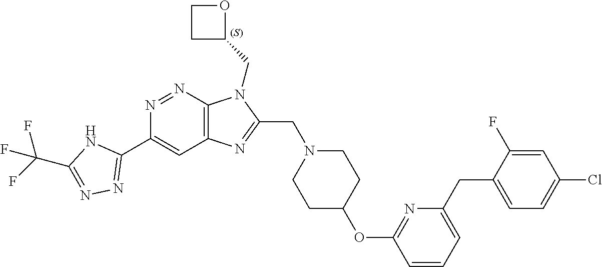 |
| 182a | 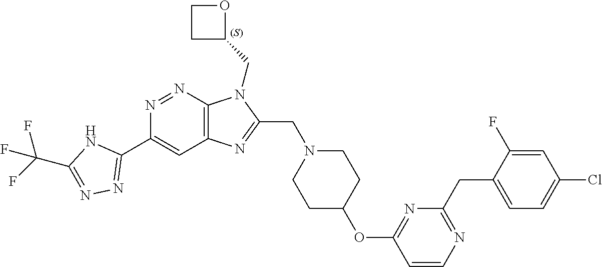 |
| 183a | 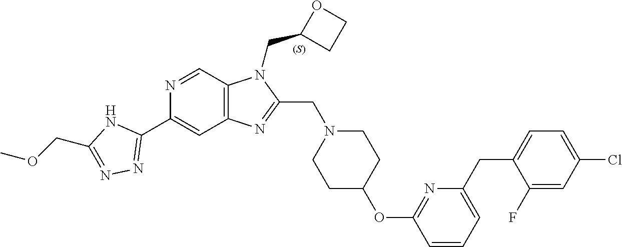 |
| 184a | 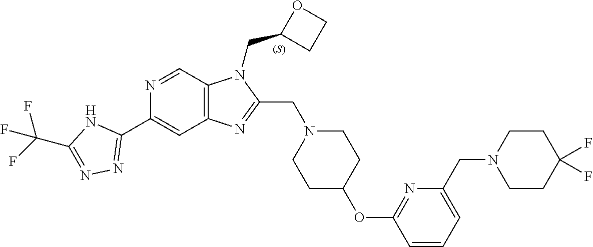 |
| 185a | 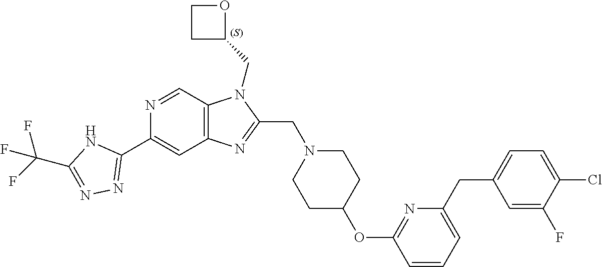 |
| 186a | 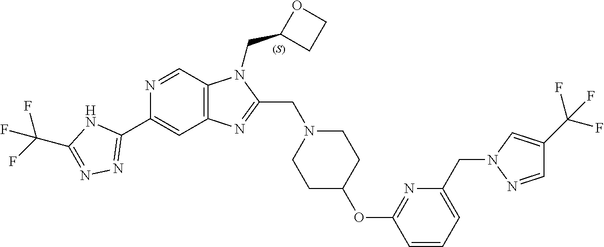 |
| 187a | 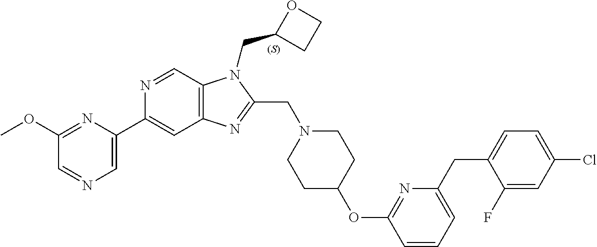 |
| 188a | 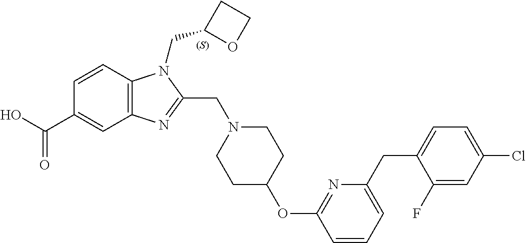 |
| 189a | 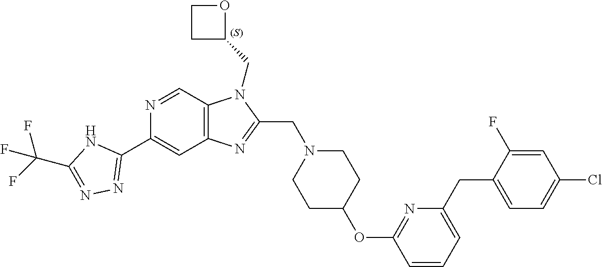 |
| 190a | 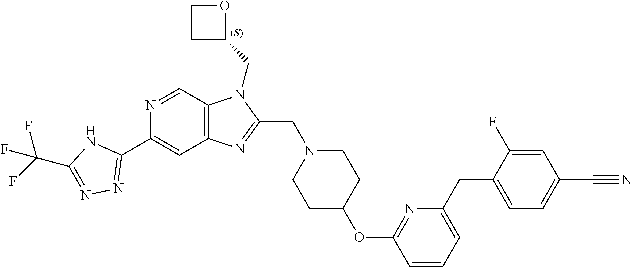 |
| 193a | 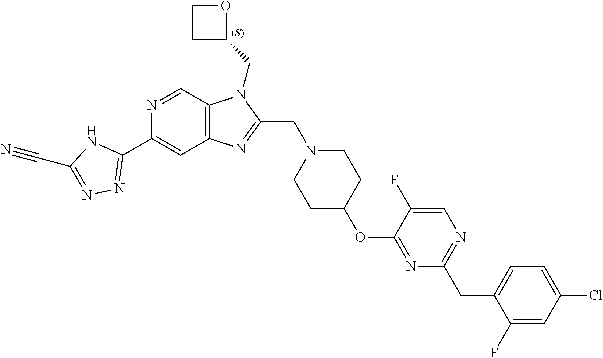 |

The compounds of Formula I include pharmaceutically acceptable salts thereof. In addition, the compounds of Formula I also include other salts of such compounds which are not necessarily pharmaceutically acceptable salts, and which may be useful as intermediates for preparing and/or purifying compounds of Formula I and/or for separating enantiomers of compounds of Formula IL Non-limiting examples of pharmaceutically acceptable salts of compounds of Formula I include trifluoroacetic acid salts.

It will further be appreciated that the compounds of Formula I or their salts may be isolated in the form of solvates, and accordingly that any such solvate is included within the scope of the present invention. For example, compounds of Formula I and salts thereof can exist in unsolvated as well as solvated forms with pharmaceutically acceptable solvents such as water, ethanol, and the like.

Pharmaceutical Compositions and Administration

When employed as pharmaceuticals, the compounds of Formula I, including pharmaceutically acceptable salts or solvates thereof can be administered in the form of a pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula I, or a pharmaceutically acceptable salt or solvate thereof, in combination with one or more pharmaceutically acceptable excipients (carriers). For example, a pharmaceutical composition prepared using a compound of Formula I, or a pharmaceutically acceptable salt or solvate thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula I, or a pharmaceutically acceptable salt or solvate thereof with a pharmaceutically acceptable excipient. Pharmaceutical compositions containing a compound of Formula I, or a pharmaceutically acceptable salt or solvate thereof as the active ingredient can be prepared by intimately mixing the compound of Formula I, or a pharmaceutically acceptable salt or solvate thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In some embodiments, the compound or pharmaceutical composition can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, UK. 2012).

In some embodiments, the compounds and pharmaceutical compositions described herein or a pharmaceutical composition thereof can be administered to patient in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal (e.g., intranasal), nasogastric, oral, parenteral, percutaneous, peridural, rectal, respiratory (inhalation), subcutaneous, sublingual, submucosal, topical, transdermal, transmucosal, transtracheal, ureteral, urethral and vaginal. In some embodiments, a preferred route of administration is parenteral (e.g., intratumoral).

In some embodiments, a compound of Formula I, or a pharmaceutically acceptable salt or solvate thereof (e.g., a compound of Formula (I-A2), or a pharmaceutically acceptable salt or solvate thereof) as described herein or pharmaceutical compositions thereof can be formulated for parenteral administration, e.g., formulated for injection via the intraarterial, intrasternal, intracranial, intravenous, intramuscular, sub-cutaneous, or intraperitoneal routes. For example, such compositions can be prepared as injectables, either as liquid solutions or suspensions; solid forms suitable for use to prepare solutions or suspensions upon the addition of a liquid prior to injection can also be prepared; and the preparations can also be emulsified. The preparation of such formulations will be known to those of skill in the art in light of the present disclosure. In some embodiments, devices are used for parenteral administration. For example, such devices may include needle injectors, microneedle injectors, needle-free injectors, and infusion techniques.

In some embodiments, the pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In some embodiments, the form must be sterile and must be fluid to the extent that it may be easily injected. In some embodiments, the form should be stable under the conditions of manufacture and storage and must be preserved against the contaminating action of microorganisms, such as bacteria and fungi.

In some embodiments, the carrier also can be a solvent or dispersion medium containing, for example, water, ethanol, polyol (for example, glycerol, propylene glycol, and liquid polyethylene glycol, and the like), suitable mixtures thereof, and vegetable oils. In some embodiments, the proper fluidity can be maintained, for example, by the use of a coating, such as lecithin, by the maintenance of the required particle size in the case of dispersion, and by the use of surfactants. In some embodiments, the prevention of the action of microorganisms can be brought about by various antibacterial and antifungal agents, for example, parabens, chlorobutanol, phenol, sorbic acid, thimerosal, and the like. In some embodiments, isotonic agents, for example, sugars or sodium chloride are included. In some embodiments, prolonged absorption of the injectable compositions can be brought about by the use in the compositions of agents delaying absorption, for example, aluminum monostearate and gelatin.

In some embodiments, sterile injectable solutions are prepared by incorporating a compound of Formula I, or a pharmaceutically acceptable salt or solvate thereof (e.g., a compound of Formula (I-A2), or a pharmaceutically acceptable salt or solvate thereof) in the required amount in the appropriate solvent with various of the other ingredients enumerated above, as required, followed by filtered sterilization. In some embodiments, dispersions are prepared by incorporating the various sterilized active ingredients into a sterile vehicle which contains the basic dispersion medium and the required other ingredients from those enumerated above. In some embodiments, sterile powders are used for the preparation of sterile injectable solutions. In some embodiments, the methods of preparation are vacuum-drying and freeze-drying techniques, which yield a powder of the active ingredient, plus any additional desired ingredient from a previously sterile-filtered solution thereof.

In some embodiments, pharmacologically acceptable excipients usable in a rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol, Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In some embodiments, suppositories can be prepared by mixing a compound of Formula I, or a pharmaceutically acceptable salt or solvate thereof (e.g., a compound of Formula (I-A2), or a pharmaceutically acceptable salt or solvate thereof) or pharmaceutical compositions as described herein with suitable non-irritating excipients or carriers such as cocoa butter, polyethylene glycol or a suppository wax which are solid at ambient temperature but liquid at body temperature and therefore melt in the rectum and release the active compound. In some embodiments, compositions for rectal administration are in the form of an enema.

In some embodiments, a compound of Formula I, or a pharmaceutically acceptable salt or solvate thereof (e.g., a compound of Formula (I-A2), or a pharmaceutically acceptable salt or solvate thereof) as described herein or a pharmaceutical composition thereof is formulated for local delivery to the digestive or GI tract by way of oral administration (e.g., solid or liquid dosage forms).

In some embodiments, solid dosage forms for oral administration include capsules, tablets, pills, powders, and granules. In some embodiments, a compound of Formula I, or a pharmaceutically acceptable salt or solvate thereof (e.g., a compound of Formula (I-A2), or a pharmaceutically acceptable salt or solvate thereof) is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For example, in the case of capsules, tablets and pills, the dosage form may also comprise buffering agents. In some embodiments, solid compositions of a similar type may also be employed as fillers in soft and hard-filled gelatin capsules using such excipients as lactose or milk sugar as well as high molecular weight polyethylene glycols and the like.

In some embodiments, the pharmaceutical compositions will take the form of a unit dosage form such as a pill or tablet and thus the composition may contain, along with a compound of Formula I, or a pharmaceutically acceptable salt or solvate thereof (e.g., a compound of Formula (I-A2), or a pharmaceutically acceptable salt or solvate thereof) as provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In some embodiments, another solid dosage form, a powder, marume, solution or suspension (e.g., in propylene carbonate, vegetable oils, PEG's, poloxamer 124 or triglycerides) is encapsulated in a capsule (gelatin or cellulose base capsule). In some embodiments, unit dosage forms in which one or more compounds and pharmaceutical compositions as provided herein or additional active agents are physically separated are also contemplated; e.g., capsules with granules (or tablets in a capsule) of each drug; two-layer tablets; two-compartment gel caps, etc. In some embodiments, enteric coated or delayed release oral dosage forms are also contemplated.

In some embodiments, other physiologically acceptable compounds may include wetting agents, emulsifying agents, dispersing agents or preservatives that are particularly useful for preventing the growth or action of microorganisms. For example, various preservatives are well known and include, for example, phenol and ascorbic acid.

In some embodiments, the excipients are sterile and generally free of undesirable matter. For example, these compositions can be sterilized by conventional, well-known sterilization techniques. In some embodiments, for various oral dosage form excipients such as tablets and capsules, sterility is not required. For example, the United States Pharmacopeia/National Formulary (USP/NF) standard can be sufficient.

In some embodiments, a compound of Formula I, or a pharmaceutically acceptable salt or solvate thereof (e.g., a compound of Formula (I-A2), or a pharmaceutically acceptable salt or solvate thereof) as described herein or a pharmaceutical composition thereof is formulated for ocular administration. In some embodiments, ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, a compound of Formula I, or a pharmaceutically acceptable salt or solvate thereof (e.g., a compound of Formula (I-A2), or a pharmaceutically acceptable salt or solvate thereof) as described herein or a pharmaceutical composition thereof is formulated for topical administration to the skin or mucosa (e.g., dermally or transdermally). In some embodiments, topical compositions can include ointments and creams. In some embodiments, ointments are semisolid preparations that are typically based on petrolatum or other petroleum derivatives. In some embodiments, creams containing the selected active agent are typically viscous liquid or semisolid emulsions, often either oil-in-water or water-in-oil. For example, cream bases are typically water-washable, and contain an oil phase, an emulsifier and an aqueous phase. For example, the oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. In some embodiments, the emulsifier in a cream formulation is generally a nonionic, anionic, cationic or amphoteric surfactant. In some embodiments, as with other carriers or vehicles, an ointment base should be inert, stable, nonirritating and non-sensitizing.

In any of the foregoing embodiments, pharmaceutical compositions as described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

In some embodiments, the dosage for a compound of Formula I, or a pharmaceutically acceptable salt or solvate thereof (e.g., a compound of Formula (I-A2), or a pharmaceutically acceptable salt or solvate thereof), is determined based on a multiple factors including, but not limited to, type, age, weight, sex, medical condition of the patient, severity of the medical condition of the patient, route of administration, and activity of the compound or pharmaceutically acceptable salt or solvate thereof. In some embodiments, proper dosage for a particular situation can be determined by one skilled in the medical arts. In some embodiments, the total daily dosage may be divided and administered in portions throughout the day or by means providing continuous delivery.

In some embodiments, a compound of Formula I, or a pharmaceutically acceptable salt or solvate thereof (e.g., a compound of Formula (I-A2), or a pharmaceutically acceptable salt or solvate thereof), is administered at a dose from about 0.01 to about 1000 mg. For example, from about 0.1 to about 30 mg, about 10 to about 80 mg, about 0.5 to about 15 mg, about 50 mg to about 200 mg, about 100 mg to about 300 mg, about 200 to about 400 mg, about 300 mg to about 500 mg, about 400 mg to about 600 mg, about 500 mg to about 800 mg, about 600 mg to about 900 mg, or about 700 mg to about 1000 mg. In some embodiments, the dose is a therapeutically effective amount.

In some embodiments, a compound of Formula I, or a pharmaceutically acceptable salt or solvate thereof (e.g., a compound of Formula (I-A2), or a pharmaceutically acceptable salt or solvate thereof) as described herein is administered at a dosage of from about 0.0002 mg/Kg to about 100 mg/Kg (e.g., from about 0.0002 mg/Kg to about 50 mg/Kg; from about 0.0002 mg/Kg to about 25 mg/Kg; from about 0.0002 mg/Kg to about 10 mg/Kg; from about 0.0002 mg/Kg to about 5 mg/Kg; from about 0.0002 mg/Kg to about 1 mg/Kg; from about 0.0002 mg/Kg to about 0.5 mg/Kg; from about 0.0002 mg/Kg to about 0.1 mg/Kg; from about 0.001 mg/Kg to about 50 mg/Kg; from about 0.001 mg/Kg to about 25 mg/Kg; from about 0.001 mg/Kg to about 10 mg/Kg; from about 0.001 mg/Kg to about 5 mg/Kg; from about 0.001 mg/Kg to about 1 mg/Kg; from about 0.001 mg/Kg to about 0.5 mg/Kg; from about 0.001 mg/Kg to about 0.1 mg/Kg; from about 0.01 mg/Kg to about 50 mg/Kg; from about 0.01 mg/Kg to about 25 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 50 mg/Kg; from about 0.1 mg/Kg to about 25 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg). In some embodiments, a compound of Formula I, or a pharmaceutically acceptable salt or solvate thereof (e.g., a compound of Formula (I-A2), or a pharmaceutically acceptable salt or solvate thereof) as described herein is administered as a dosage of about 100 mg/Kg.

In some embodiments, the foregoing dosages of a compound of Formula I, or a pharmaceutically acceptable salt or solvate thereof (e.g., a compound of Formula (I-A2), or a pharmaceutically acceptable salt or solvate thereof), can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound of Formula I, or a pharmaceutically acceptable salt or solvate thereof (e.g., a compound of Formula (I-A2), or a pharmaceutically acceptable salt or solvate thereof) as described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a compound of Formula I, or a pharmaceutically acceptable salt or solvate thereof (e.g., a compound of Formula (I-A2), or a pharmaceutically acceptable salt or solvate thereof) is administered to a patient for a period of time followed by a separate period of time where administration of the compound of Formula I, or a pharmaceutically acceptable salt or solvate thereof (e.g., a compound of Formula (I-A2), or a pharmaceutically acceptable salt or solvate thereof) is stopped. In some embodiments, a compound of Formula I, or a pharmaceutically acceptable salt or solvate thereof (e.g., a compound of Formula (I-A2), or a pharmaceutically acceptable salt or solvate thereof) is administered for a first period and a second period following the first period, with administration stopped during the second period, followed by a third period where administration of the compound of Formula I, or a pharmaceutically acceptable salt or solvate thereof (e.g., a compound of Formula (I-A2), or a pharmaceutically acceptable salt or solvate thereof) is started and then a fourth period following the third period where administration is stopped. For example, the period of administration of a compound of Formula I, or a pharmaceutically acceptable salt or solvate thereof (e.g., a compound of Formula (I-A2), or a pharmaceutically acceptable salt or solvate thereof) followed by a period where administration is stopped is repeated for a determined or undetermined period of time. In some embodiments, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In some embodiments, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, a compound of Formula I, or a pharmaceutically acceptable salt or solvate thereof (e.g., a compound of Formula (I-A2), or a pharmaceutically acceptable salt or solvate thereof), is orally administered to the patient one or more times per day (e.g., one time per day, two times per day, three times per day, four times per day per day or a single daily dose).

In some embodiments, a compound of Formula I, or a pharmaceutically acceptable salt or solvate thereof (e.g., a compound of Formula (I-A2), or a pharmaceutically acceptable salt or solvate thereof), is administered by parenteral administration to the patient one or more times per day (e.g., 1 to 4 timesone time per day, two times per day, three times per day, four times per day or a single daily dose).

In some embodiments, a compound of Formula I, or a pharmaceutically acceptable salt or solvate thereof (e.g., a compound of Formula (I-A2), or a pharmaceutically acceptable salt or solvate thereof), is administered by parenteral administration to the patient weekly.

Methods of Treatment

In some embodiments, this disclosure features methods for treating a patient (e.g., a human) having a disease, disorder, or condition in which modulation of GLP-1R (e.g., repressed or impaired and/or elevated or unwanted GLP-1R) is beneficial for the treatment of the underlying pathology and/or symptoms and/or progression of the disease, disorder, or condition. In some embodiments, the methods described herein can include or further include treating one or more conditions associated, co-morbid or sequela with any one or more of the conditions described herein.

Provided herein is a method for treating a GLP-1 associated disease, disorder, or condition, the method comprising administering to a patient in need thereof an effective amount of a compound of Formula I, or a pharmaceutically acceptable salt or solvate thereof (e.g., a compound of Formula (I-A2), or a pharmaceutically acceptable salt or solvate thereof), or a pharmaceutical composition as disclosed herein.

In some embodiments, the disease, disorder, or condition includes, but is not limited to type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, and Polycystic Ovary Syndrome (PCOS).

In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

In some embodiments, the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.

In some embodiments, the compounds and pharmaceutical compositions and methods for treating a patient described herein induce one or more of blood glucose reduction (e.g., reduce blood glucose levels), reduce blood hemoglobin A1c (HbA1c) levels, promote insulin synthesis, stimulate insulin secretion, increase the mass of β-cells, modulate gastric acid secretion, modulate gastric emptying, decrease the body mass index (BMI), and/or decrease glucagon production (e.g., level). In certain embodiments, the compounds and pharmaceutical compositions and methods for treating a patient described herein stabilize serum glucose and serum insulin levels (e.g., serum glucose and serum insulin concentrations). Also provided herein are methods for modulating glucose or insulin levels in a patient in need of such modulating, the method comprising administering to the patient an effective amount of a compound of Formula I, or a pharmaceutically acceptable salt or solvate thereof (e.g., a compound of Formula (I-A2), or a pharmaceutically acceptable salt or solvate thereof), or a pharmaceutical composition as disclosed herein.

In some embodiments, provided herein is a method for reducing the risk (e.g., by about at least 20%, at least 30%, at least 40%, at least 50%, at least 60%, at least 70%, or at least 80%) of major adverse cardiovascular events (MACE) in a patient in need thereof, the method comprising administering to the patient an effective amount of a compound of Formula I, or a pharmaceutically acceptable salt or solvate thereof (e.g., a compound of Formula (I-A2), or a pharmaceutically acceptable salt or solvate thereof), or a pharmaceutical composition as disclosed herein. In certain of these embodiments, the patient is an adult that has been diagnosed with type 2 diabetes (T2D). In certain embodiments, the patient is an adult that has been diagnosed with a heart disease. In certain embodiments, the patient is an adult that has been diagnosed with type 2 diabetes (T2D) and a heart disease. In certain embodiments, the patient is an adult that has type 2 diabetes (T2D). In certain embodiments, the patient is an adult that has a heart disease. In certain embodiments, the patient has type 2 diabetes (T2D) and a heart disease.

Indications

Obesity

In some embodiments, the condition, disease or disorder is obesity and conditions, diseases or disorders that are associated with or related to obesity. Non-limiting examples of obesity and obesity related conditions include symptomatic obesity, simple obesity, childhood obesity, morbid obesity, and abdominal obesity (central obesity characterized by abdominal adiposity). Non-limiting examples of symptomatic obesity include endocrine obesity (e.g., Cushing syndrome, hypothyroidism, insulinoma, obese type II diabetes, pseudohypoparathyroidism, hypogonadism), hypothalamic obesity, hereditary obesity (e.g., Prader-Willi syndrome, Laurence-Moon-Biedl syndrome), and drug-induced obesity (e.g., steroid, phenothiazine, insulin, sulfonylurea agent, or β-blocker-induced obesity).

In some embodiments, the condition, disease or disorder is associated with obesity. Examples of such conditions, diseases or disorders include, without limitation, glucose tolerance disorders, diabetes (e.g., type 2 diabetes, obese diabetes), lipid metabolism abnormality, hyperlipidemia, hypertension, cardiac failure, hyperuricemia, gout, fatty liver (including non-alcoholic steatohepatitis (NASH)), coronary heart disease (e.g., myocardial infarction, angina pectoris), cerebral infarction (e.g., brain thrombosis, transient cerebral ischemic attack), bone or articular disease (e.g., knee osteoarthritis, hip osteoarthritis, spondylitis deformans, lumbago), sleep apnea syndrome, obesity hypoventilation syndrome (Pickwickian syndrome), menstrual disorder (e.g., abnormal menstrual cycle, abnormality of menstrual flow and cycle, amenorrhea, abnormal catamenial symptom), visceral obesity syndrome, and metabolic syndrome. In some embodiments, the chemical compound and pharmaceutical compositions described herein can be used to treat patients exhibiting symptoms of both obesity and insulin deficiency.

Diabetes

In some embodiments, the condition, disease or disorder is diabetes. Non-limiting examples of diabetes include type 1 diabetes mellitus, type 2 diabetes mellitus (e.g., diet-treated type 2-diabetes, sulfonylurea-treated type 2-diabetes, a far-advanced stage type 2-diabetes, long-term insulin-treated type 2-diabetes), diabetes mellitus (e.g., non-insulin-dependent diabetes mellitus, insulin-dependent diabetes mellitus), gestational diabetes, obese diabetes, autoimmune diabetes, and borderline type diabetes. In some embodiments, the condition, disease or disorder is type 2 diabetes mellitus (e.g., diet-treated type 2-diabetes, sulfonylurea-treated type 2-diabetes, a far-advanced stage type 2-diabetes, long-term insulin-treated type 2-diabetes).

Provided herein is a method of treating a diabetes mellitus in a patient, the method comprising (a) determining that the patient has type 2 diabetes mellitus, and (b) administering to the patient a therapeutically effective amount of a compound of Formula I, or a pharmaceutically acceptable salt or solvate thereof (e.g., a compound of Formula (I-A2), or a pharmaceutically acceptable salt or solvate thereof, or a pharmaceutically acceptable salt or solvate thereof) or a pharmaceutical composition as disclosed herein.

Provided herein is a method for treating type 2 diabetes mellitus in a patient, the method comprising administering to a patient identified or diagnosed as having type 2 diabetes mellitus a therapeutically effective amount of a compound of Formula I, or a pharmaceutically acceptable salt or solvate thereof (e.g., a compound of Formula (I-A2), or a pharmaceutically acceptable salt or solvate thereof, or a pharmaceutically acceptable salt or solvate thereof), or a pharmaceutical composition as disclosed herein.

Also provided herein is a method of treating type 2 diabetes mellitus in a patient in need thereof, the method comprising administering to the patient a therapeutically effective amount of a compound of Formula I, or a pharmaceutically acceptable salt or solvate thereof (e.g., a compound of Formula (I-A2), or a pharmaceutically acceptable salt or solvate thereof, or a pharmaceutically acceptable salt or solvate thereof), or a pharmaceutical composition as disclosed herein.

In some embodiments, the compounds and pharmaceutical compositions and methods for treating a patient with a condition, disease, or disorder (e.g., type 2 diabetes mellitus) described herein reduce fasting plasma glucose levels. In some embodiments, the compounds and pharmaceutical compositions and methods for treating a patient with a condition, disease, or disorder (e.g., type 2 diabetes mellitus) described herein reduce non-fasting plasma glucose levels. In some embodiments, the compounds and pharmaceutical compositions and methods for treating a patient with a condition, disease, or disorder (e.g., type 2 diabetes mellitus) described herein reduce HbA1c levels. In some embodiments, the compounds and pharmaceutical compositions and methods for treating a patient with a condition, disease, or disorder (e.g., type 2 diabetes mellitus) described herein reduce glucagon levels. In some embodiments, the compounds and pharmaceutical compositions and methods for treating a patient with a condition, disease, or disorder (e.g., type 2 diabetes mellitus) described herein increase insulin levels. In some embodiments, the compounds and pharmaceutical compositions and methods for treating a patient with a condition, disease, or disorder (e.g., type 2 diabetes mellitus) described herein reduce BMI.

In some embodiments, a reduction in fasting plasma glucose levels of about 5% to about 95% indicates treatment of type 2 diabetes mellitus. In some embodiments, a reduction in fasting plasma glucose levels of about 15% to about 80% indicates treatment of type 2 diabetes mellitus. In some embodiments, a reduction in fasting plasma glucose levels of about 25% to about 60% indicates treatment of type 2 diabetes mellitus. In some embodiments, a reduction in fasting plasma glucose levels to about or below 126 mg/dL, about or below 110 mg/dL, or about or below 90 mg/dL indicates treatment of the type 2 diabetes mellitus.

In some embodiments, a reduction in non-fasting plasma glucose levels of about 5% to about 95% indicates treatment of type 2 diabetes mellitus. In some embodiments, a reduction in non-fasting plasma glucose levels of about 15% to about 80% indicates treatment of type 2 diabetes mellitus. In some embodiments, a reduction in non-fasting plasma glucose levels of about 25% to about 60% indicates treatment of type 2 diabetes mellitus. In some embodiments, a reduction in non-fasting plasma glucose levels to about or below 200 mg/dL, about or below 150 mg/dL, or about or below 130 mg/dL indicates treatment of type 2 diabetes mellitus.

In some embodiments, a reduction in HbA1c levels of about 5% to about 95% indicates treatment of type 2 diabetes mellitus. In some embodiments, a reduction in HbA1c levels of about 15% to about 80% indicates treatment of type 2 diabetes mellitus. In some embodiments, a reduction in HbA1c levels of about 25% to about 60% indicates treatment of type 2 diabetes mellitus. In some embodiments, reduction in HbA1c levels to about or below 6.5%, about or below 6.0%, or about or below 5.0% indicates treatment of type 2 diabetes mellitus.

In some embodiments, a reduction in glucagon levels of about 5% to about 95% indicates treatment of type 2 diabetes mellitus. In some embodiments, a reduction in glucagon levels of about 15% to about 80% indicates treatment of type 2 diabetes mellitus. In some embodiments, a reduction in glucagon levels of about 25% to about 60% indicates treatment of type 2 diabetes mellitus. In some embodiments, an increase in insulin levels of about 5% to about 95% indicates treatment of type 2 diabetes mellitus. In some embodiments, an increase in insulin levels of about 15% to about 80% indicates treatment of type 2 diabetes mellitus. In some embodiments, an increase in insulin levels of about 25% to about 60% indicates treatment of type 2 diabetes mellitus.

In some embodiments, a reduction in BMI of about 5% to about 95% indicates treatment of type 2 diabetes mellitus. In some embodiments, a reduction in BMI of about 15% to about 80% indicates treatment of the type 2 diabetes mellitus. In some embodiments, a reduction in BMI of about 25% to about 60% indicates treatment of type 2 diabetes mellitus. In some embodiments, a reduction in BMI of about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% indicates treatment of type 2 diabetes mellitus. In some embodiments, a reduction in BMI to about or below 40, about or below 30, or about or below 20 indicates treatment of type 2 diabetes mellitus.

In some embodiments, the condition, disease or disorder is associated with diabetes (e.g., a complication of diabetes). Non-limiting examples of disorders associated with diabetes include obesity, obesity-related disorders, metabolic syndrome, neuropathy, nephropathy (e.g., diabetic nephropathy), retinopathy, diabetic cardiomyopathy, cataract, macroangiopathy, osteopenia, hyperosmolar diabetic coma, infectious disease (e.g., respiratory infection, urinary tract infection, gastrointestinal infection, dermal soft tissue infections, inferior limb infection), diabetic gangrene, xerostomia, hypacusis, cerebrovascular disorder, diabetic cachexia, delayed wound healing, diabetic dyslipidemia peripheral blood circulation disorder, cardiovascular risk factors. (e.g., coronary artery disease, peripheral artery disease, cerebrovascular disease, hypertension, and risk factors related to unmanaged cholesterol and/or lipid levels, and/or inflammation), NASH, bone fracture, and cognitive dysfunction

Other non-limiting examples of disorders related to diabetes include pre-diabetes, hyperlipidemia (e.g., hypertriglyceridemia, hypercholesterolemia, high LDL-cholesterolemia, low HDL-cholesterolemia, postprandial hyperlipemia), metabolic syndrome (e.g., metabolic disorder where activation of GLP-1R is beneficial, metabolic syndrome X), hypertension, impaired glucose tolerance (IGT), insulin resistance, and sarcopenia.

In some embodiments, the condition, disease or disorder is diabetes and obesity (diabesity). In some embodiments, the compounds described herein are also useful in improving the therapeutic effectiveness of metformin.

Disorders of Metabolically Important Tissues

In some embodiments, the condition, disease or disorder is a disorder of a metabolically important tissue. Non-limiting examples of metabolically important tissues include liver, fat, pancreas, kidney, and gut.

In some embodiments, the condition, disease or disorder is a fatty liver disease. Fatty liver diseases include, but are not limited to, non-alcoholic fatty acid liver disease (NAFLD), steatohepatitis, non-alcoholic steatohepatitis (NASH), fatty liver disease resulting from hepatitis, fatty liver disease resulting from obesity, fatty liver disease resulting from diabetes, fatty liver disease resulting from insulin resistance, fatty liver disease resulting from hypertriglyceridemia, Abetalipoproteinemia, glycogen storage diseases, Weber-Christian disease, Wolmans disease, acute fatty liver of pregnancy, and lipodystrophy.

Non-alcoholic fatty liver disease (NAFLD) represents a spectrum of disease occurring in the absence of alcohol abuse and is typically characterized by the presence of steatosis (fat in the liver). NAFLD is believed to be linked to a variety of conditions, e.g., metabolic syndrome (including obesity, diabetes and hypertriglyceridemia) and insulin resistance. It can cause liver disease in adults and children and may ultimately lead to cirrhosis (Skelly et al., J Hepatol 2001; 35: 195-9; Chitturi et al., Hepatology 2002; 35(2):373-9). The severity of NAFLD ranges from the relatively benign isolated predominantly macrovesicular steatosis (i.e., nonalcoholic fatty liver or NAFL) to non-alcoholic steatohepatitis (NASH) (Angulo et al., J Gastroenterol Hepatol 2002; 17 Suppl:S186-90). In some embodiments, the patient is a pediatric patient. The term “pediatric patient” as used herein refers to a patient under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age. In some embodiments, the patient is an adult patient.

Other non-limiting examples of disorders in metabolically important tissues include joint disorders (e.g., osteoarthritis, secondary osteoarthritis), steatosis (e.g. in the liver); gall stones; gallbladder disorders; gastroesophageal reflux; sleep apnea; hepatitis; fatty liver; bone disorder characterized by altered bone metabolism, such as osteoporosis, including post-menopausal osteoporosis, poor bone strength, osteopenia, Paget's disease, osteolytic metastasis in cancer patients, osteodistrophy in liver disease and the altered bone metabolism caused by renal failure or haemodialysis, bone fracture, bone surgery, aging, pregnancy, protection against bone fractures, and malnutritionpolycystic ovary syndrome; renal disease (e.g., chronic renal failure, glomerulonephritis, glomerulosclerosis, nephrotic syndrome, hypertensive nephrosclerosis, end-stage renal disease); muscular dystrophy, angina pectoris, acute or chronic diarrhea, testicular dysfunction, respiratory dysfunction, frailty, sexual dysfunction (e.g., erectile dysfunction), and geriatric syndrome. In some embodiments, the compounds and pharmaceutical compositions described herein can be used for treating surgical trauma by improving recovery after surgery and/or by preventing the catabolic reaction caused by surgical trauma.

Cardiovascular and Vascular Diseases

In some embodiments, the condition, disease or disorder is a cardiovascular disease. Non-limiting examples of cardiovascular disease include congestive heart failure, atherosclerosis, arteriosclerosis, coronary heart disease, coronary artery disease, congestive heart failure, coronary heart disease, hypertension, cardiac failure, cerebrovascular disorder (e.g., cerebral infarction), vascular dysfunction, myocardial infarction, elevated blood pressure (e.g., 130/85 mm Hg or higher), and prothrombotic state (exemplified by high fibrinogen or plasminogen activator inhibitor in the blood).

In some embodiments, the condition, disease or disorder is related to a vascular disease. Non-limiting examples of vascular diseases include peripheral vascular disease, macrovascular complications (e.g., stroke), vascular dysfunction, peripheral artery disease, abdominal aortic aneurysm, carotid artery disease, cerebrovascular disorder (e.g., cerebral infarction), pulmonary embolism, chronic venous insufficiency, critical limb ischemia, retinopathy, nephropathy, and neuropathy.

Neurological Diseases

In some embodiments, the condition, disease or disorder is a neurological disorder (e.g., neurodegenerative disorder) or a psychiatric disorder. Non-limiting examples of neurological disorders include brain insulin resistance, mild cognitive impairment (MCI), Alzheimer's disease (AD), Parkinson's disease (PD), anxiety, dementia (e.g., senile dementia), traumatic brain injury, Huntington's chores, tardive dyskinesia, hyperkinesia, mania, Morbus Parkinson, steel-Richard syndrome, Down's syndrome, myasthenia gravis, nerve trauma, brain trauma, vascular amyloidosis, cerebral hemorrhage I with amyloidosis, brain inflammation, Friedrich's ataxia, acute confusion disorder, amyotrophic lateral sclerosis (ALS), glaucoma, and apoptosis-mediated degenerative diseases of the central nervous system (e.g., Creutzfeld-Jakob Disease, bovine spongiform encephalopathy (mad cow disease), and chronic wasting syndrome). See, e.g., US20060275288A1.

Non-limiting examples of psychiatric disorders include drug dependence/addiction (narcotics and amphetamines and attention deficit/hyperactivity disorder (ADHD). The compounds and pharmaceutical compositions described herein can be useful in improving behavioral response to addictive drugs, decreasing drug dependence, prevention drug abuse relapse, and relieving anxiety caused by the absence of a given addictive substance. See, e.g., US20120021979A1.

In some embodiments, the compounds and pharmaceutical compositions described herein are useful in improving learning and memory by enhancing neuronal plasticity and facilitation of cellular differentiation, and also in preserving dopamine neurons and motor function in Morbus Parkinson.

Insulin-Related

In some embodiments, the condition, disease or disorder is impaired fasting glucose (IFG), impaired fasting glycemia (IFG), hyperglycemia, insulin resistance (impaired glucose homeostasis), hyperinsulinemia, elevated blood levels of fatty acids or glycerol, a hypoglycemic condition, insulin resistant syndrome, paresthesia caused by hyperinsulinemia, hyperlipidaemia, hypercholesteremia, impaired wound healing, leptin resistance, glucose intolerance, increased fasting glucose, dyslipidemia (e.g., hyperlipidemia, atherogenic dyslipidemia characterized by high triglycerides and low HDL cholesterol), glucagonoma, hyperuricacidemia, hypoglycemia (e.g., nighttime hypoglycemia), and concomitant comatose endpoint associated with insulin.

In some embodiments, the compounds and pharmaceutical compositions described herein can reduce or slow down the progression of borderline type, impaired fasting glucose or impaired fasting glycemia into diabetes.

Autoimmune Disorders

In some embodiments, the condition, disease or disorder is an autoimmune disorder. Non-limiting examples of autoimmune disorders include multiple sclerosis, experimental autoimmune encephalomyelitis, autoimmune disorder is associated with immune rejection, graft versus host disease, uveitis, optic neuropathies, optic neuritis, transverse myelitis, inflammatory bowel disease, rheumatoid arthritis, ankylosing spondylitis, systemic lupus erythematosus, myasthenia gravis, and Graves disease. See, e.g., US20120148586A1.

Stomach and Intestine-Related Disorders

In some embodiments, the condition, disease or disorder is a stomach or intestine related disorder. Non-limiting examples of these disorders include ulcers of any etiology (e.g. peptic ulcers, Zollinger-Ellison syndrome, drug-induced ulcers, ulcers related to infections or other pathogens), digestion disorders, malabsorption, short bowel syndrome, cul-de-sac syndrome, inflammatory bowel diseases (Crohn's disease and ulcerative colitis), celiac sprue, hypogammaglobulinemic sprue, chemotherapy and/or radiation therapy-induced mucositis and diarrhea, gastrointestinal inflammation, short bowel syndrome, colitis ulcerosa, gastric mucosal injury (e.g., gastric mucosal injury caused by aspirin), small intestinal mucosal injury, and cachexia (e.g., cancerous cachexia, tuberculous cachexia, cachexia associated with blood disease, cachexia associated with endocrine disease, cachexia associated with infectious disease, and cachexia caused by acquired immunodeficiency syndrome).

Body Weight

In some embodiments, the compounds and pharmaceutical compositions described herein can be used to reduce body weight (e.g., excess body weight), prevent body weight gain, induce weight loss, decrease body fat, or reduce food intake in a patient (e.g., a patient in need thereof). In some embodiments, the weight increase in a patient may be attributed to excessive ingestion of food or unbalanced diets, or may be weight increase derived from a concomitant drug (e.g., insulin sensitizers having a PPARγ agonist-like action, such as troglitazone, rosiglitazone, englitazone, ciglitazone, pioglitazone and the like). In some embodiments, the weight increase may be weight increase before reaching obesity, or may be weight increase in an obese patient. In some embodiments, the weight increase may also be medication-induced weight gain or weight gain subsequent to cessation of smoking.

In some embodiments, the condition, disease or disorder is an eating disorder, such as hyperphagia, binge eating, bulimia, or compulsive eating.

Inflammatory Diseases

In some embodiments, the condition, disease or disorder is an inflammatory disorder. Non-limiting examples of inflammatory disorders include chronic rheumatoid arthritis, spondylitis deformans, arthritis deformans, lumbago, gout, post-operational or post-traumatic inflammation, bloating, neuralgia, laryngopharyngitis, cystitis, pneumonia, pancreatitis, enteritis, inflammatory bowel disease (including inflammatory large bowel disease), inflammation in metabolically important tissues including liver, fat, pancreas, kidney and gut, and a proinflammatory state (e.g., elevated levels of proinflammatory cytokines or markers of inflammation-like C-reactive protein in the blood).

Cancer

In some embodiments, the condition, disease or disorder is cancer. Suitable examples of cancer include breast cancer (e.g., invasive ductal breast cancer, noninvasive ductal breast cancer, inflammatory breast cancer), prostate cancer (e.g., hormone-dependent prostate cancer, hormone-independent prostate cancer), pancreatic cancer (e.g., ductal pancreatic cancer), gastric cancer (e.g., papillary adenocarcinoma, mucous adenocarcinoma, adenosquamous carcinoma), lung cancer (e.g., non-small cell lung cancer, small-cell lung cancer, malignant mesothelioma), colon cancer (e.g., gastrointestinal stromal tumor), rectal cancer (e.g., gastrointestinal stromal tumor), colorectal cancer (e.g., familial colorectal cancer, hereditary non-polyposis colorectal cancer, gastrointestinal stromal tumor), small intestinal cancer (e.g., non-Hodgkin's lymphoma, gastrointestinal stromal tumor), esophageal cancer, duodenal cancer, tongue cancer, pharyngeal cancer (e.g., nasopharyngeal cancer, oropharynx cancer, hypopharyngeal cancer), salivary gland cancer, brain tumor (e.g., pineal astrocytoma, pilocytic astrocytoma, diffuse astrocytoma, anaplastic astrocytoma), neurilemmoma, liver cancer (e.g., primary liver cancer, extrahepatic bile duct cancer), renal cancer (e.g., renal cell cancer, transitional cell cancer of the renal pelvis and ureter), bile duct cancer, endometrial cancer, uterine cervical cancer, ovarian cancer (e.g., epithelial ovarian cancer, extragonadal germ cell tumor, ovarian germ cell tumor, ovarian tumor of low malignant potential), bladder cancer, urethral cancer, skin cancer (e.g., intraocular (ocular) melanoma, Merkel cell carcinoma), hemangioma, malignant lymphoma, malignant melanoma, thyroid cancer (e.g., medullary thyroid cancer), parathyroid cancer, nasal cavity cancer, sinus cancer, bone tumor (e.g., osteosarcoma, Ewing tumor, uterine sarcoma, soft tissue sarcoma), angiofibroma, sarcoma of the retina, penis cancer, testicular tumor, pediatric solid tumor (e.g., Wilms' tumor, childhood kidney tumor), Kaposi's sarcoma, Kaposi's sarcoma caused by AIDS, tumor of maxillary sinus, fibrous histiocytoma, leiomyosarcoma, rhabdomyosarcoma, and leukemia (e.g., acute myeloid leukemia, acute lymphoblastic leukemia).

Hypothalamic-Pituitary Disorders

In some embodiments, the condition, disease or disorder is related to the hypothalamic-pituitary-gonadal axis. For example, the condition, disease or disorder is related to the hypothalamus-pituitary-ovary axis. In another example, the condition, disease or disorder is related to the hypothalamus-pituitary-testis axis. Hypothalamic-pituitary-gonadal axis diseases include, but are not limited to, hypogonadism, polycystic ovary syndrome, hypothyroidism, hypopituitarism, sexual dysfunction, and Cushing's disease.

In some embodiments, the condition, disease or disorder associated with diabetes is related to the hypothalamic-pituitary-gonadal axis.

Pulmonary Disease

In some embodiments, the condition, disease or disorder is related to a pulmonary disease. Pulmonary diseases include, but are not limited to, asthma, idiopathic pulmonary fibrosis, pulmonary hypertension, obstructive sleep apnoea-hypopnoea syndrome, and chronic obstructive pulmonary disease (COPD) (e.g., emphysema, chronic bronchitis, and refractory (non-reversible) asthma).

In some embodiments, the condition, disease or disorder associated with diabetes is a pulmonary disease.

Combination Therapy

In some embodiments, this disclosure contemplates both monotherapy regimens as well as combination therapy regimens.

In some embodiments, the methods described herein can further include administering one or more additional therapies (e.g., one or more additional therapeutic agents and/or one or more therapeutic regimens) in combination with administration of the compounds described herein.

In some embodiments, the methods described herein include administering a compound described herein in combination with one or more of a diet therapy (e.g., dietary monitoring, diet therapy for diabetes), an exercise therapy (e.g., physical activity), blood sugar monitoring, gastric electrical stimulation (e.g., TANTALUS®), and diet modifications.

In some embodiments, the compounds of X, or a pharmaceutically acceptable salt or solvate thereof as described herein can be administered in combination with one or more additional therapeutic agents.

Representative additional therapeutic agents include, but are not limited to, anti-obesity agents, therapeutic agents for diabetes, therapeutic agents for diabetic complications, therapeutic agents for hyperlipidemia, antihypertensive agents, diuretics, chemotherapeutics, immunotherapeutics, anti-inflammatory drugs, antithrombotic agents, anti-oxidants, therapeutic agents for osteoporosis, vitamins, antidementia drugs, erectile dysfunction drugs, therapeutic drugs for urinary frequency or urinary incontinence, therapeutic agents for NAFLD, therapeutic agents for NASH, therapeutic agents for dysuria and anti-emetic agents.

In some embodiments, the one or more additional therapeutic agents include those useful, for example, as anti-obesity agents. Non-limiting examples include monoamine uptake inhibitors (e.g., tramadol, phentermine, sibutramine, mazindol, fluoxetine, tesofensine), serotonin 2C receptor agonists (e.g., lorcaserin), serotonin 6 receptor antagonists, histamine H3 receptor modulator, GABA modulator (e.g., topiramate), including GABA receptor agonists (e.g., gabapentin, pregabalin), neuropeptide Y antagonists (e.g., velneperit), cannabinoid receptor antagonists (e.g., rimonabant, taranabant), ghrelin antagonists, ghrelin receptor antagonists, ghrelin acylation enzyme inhibitors, opioid receptor antagonists (e.g., GSK-1521498), orexin receptor antagonists, melanocortin 4 receptor agonists, 11β-hydroxysteroid dehydrogenase inhibitors (e.g., AZD-4017, BVT-3498, INCB-13739), pancreatic lipase inhibitors (e.g., orlistat, cetilistat), β3 agonists (e.g., N-5984), diacylglycerol acyltransferase 1 (DGAT1) inhibitors, acetylCoA carboxylase (ACC) inhibitors, stearoyl-CoA desaturated enzyme inhibitors, microsomal triglyceride transfer protein inhibitors (e.g., R-256918), sodium-glucose cotransporter 2 (SGLT-2) inhibitors (e.g., JNJ-28431754, dapagliflozin, AVE2268, TS-033, YM543, TA-7284, ASP1941, remogliflozin), NFK inhibitors (e.g., HE-3286), PPAR agonists (e.g., GFT-505, DRF-11605, gemfibrozil and fenofibrate), phosphotyrosine phosphatase inhibitors (e.g., sodium vanadate, trodusquemin), GPR119 agonists (e.g., PSN-821, MBX-2982, APD597), glucokinase activators (e.g., piragliatin, AZD-1656, AZD6370, TTP-355, compounds described in W0006/112549, W0007/028135, W0008/047821, W0008/050821, W0008/136428 and W0008/156757), leptin, leptin derivatives (e.g., metreleptin), leptin resistance improving drugs, CNTF (ciliary neurotrophic factor), BDNF (brain-derived neurotrophic factor), cholecystokinin agonists, amylin preparations (e.g., pramlintide, AC-2307), neuropeptide Y agonists (e.g., PYY3-36, derivatives of PYY3-36, obineptide, TM-30339, TM-30335), oxyntomodulin (OXM) preparations, appetite suppressants (e.g. ephedrine), FGF21 preparations (e.g., animal FGF21 preparations extracted from the pancreas of bovine or swine; human FGF21 preparations genetically synthesized using Escherichia coli or yeast; fragments or derivatives of FGF21), anorexigenic agents (e.g., P-57), human proislet peptide (HIP), farnesoid X receptor (FXR) agonist, phentermine, zonisamide, norepinephrine/dopamine reuptake inhibitor, GDF-15 analog, methionine aminopeptidase 2 (MetAP2) inhibitor, diethylpropion, phendimetrazine, benzphetamine, fibroblast growth factor receptor (FGFR) modulator, and AMP-activated protein kinase (AMPK) activator.

In some embodiments, the one or more additional therapeutic agents include those useful, for example, as anti-diabetic agents. Non-limiting examples include insulin and insulin preparations (e.g., animal insulin preparations extracted from the pancreas of bovine or swine; human insulin preparations genetically synthesized using Escherichia coli or yeast; zinc insulin; protamine zinc insulin; fragment or derivative of insulin (e.g., INS-1), oral insulin preparation, synthetic human insulin), insulin sensitizers (e.g., pioglitazone or a salt thereof), biguanides (e.g., metformin, buformin or a salt thereof (e.g., hydrochloride, fumarate, succinate)), glucagon analogs (e.g., any of glucagon analogs described, e.g., in WO 2010/011439), agents which antagonize the actions of or reduce secretion of glucagon, sulfonylurea agents (e.g., chlorpropamide, tolazamide, gliclazide, glimepiride, tolbutamide, glibenclamide, gliclazide, acetohexamide, glyclopyramide, glybuzole, glyburide), thiazolidinedione agents (e.g. rosiglitazone or pioglitazone), α-glucosidase inhibitors (e.g., voglibose, acarbose, miglitol, emiglitate), insulin secretagogues, such as prandial glucose regulators (sometimes called “short-acting secretagogues”), e.g., meglitinides (e.g. repaglinide and nateglinide), cholinesterase inhibitors (e.g., donepezil, galantamine, rivastigmine, tacrine), NMDA receptor antagonists, dual GLP-1/GIP receptor agonists (e.g., LBT-2000, ZPD1-70), GLP-1R agonists (e.g., exenatide, liraglutide, albiglutide, dulaglutide, abiglutide, taspoglutide, lixisenatide, semaglutide, AVE-0010, S4P and Boc5), and dipeptidyl peptidase IV (DPP-4) inhibitors (e.g., vildagliptin, dutogliptin, gemigliptin, alogliptin, saxagliptin, sitagliptin, linagliptin, berberine, adogliptin, B¹¹³⁵⁶, GRC8200, MP-513, PF-00734200, PHX1149, SK-0403, ALS2-0426, TA-6666, TS-021, KRP-104, trelagliptin).

In some embodiments, the one or more additional therapeutic agents include those useful, for example, for treating NAFL and NASH. Non-limiting examples include FXR agonists, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21), a niacin analogue, a leukotriene D4 (LTD4) receptor antagonist, an afcetyl-CoA carboxylase (ACC) inhibitor, a ketohexokinase (KHK) inhibitor, an apoptosis signal-regulating kinase 1 (ASK1) inhibitor, an ileal bile acid transporter (IBAT) inhibitor, glycyrrhizin, schisandra extract, ascorbic acid, glutathione, silymarin, lipoic acid, and d-alpha-tocopherol, ascorbic acid, glutathione, vitamin B-complex, glitazones/thiazolidinediones (e.g., troglitazone, rosiglitazone, pioglitazone), metformin, cysteamine, sulfonylureas, alpha-glucosidase inhibitors, meglitinides, vitamin E, tetrahydrolipstatin, milk thistle protein, anti-virals, and anti-oxidants.

In some embodiments, the one or more additional therapeutic agents include those useful, for example, for treating diabetic complications. Non-limiting examples include aldose reductase inhibitors (e.g., tolrestat, epalrestat, zopolrestat, fidarestat, CT-112, ranirestat, lidorestat), neurotrophic factor and increasing agents thereof (e.g., NGF, NT-3, BDNF, neurotrophic production/secretion promoting agents described in WO01/14372 (e.g., 4-(4-chlorophenyl)-2-(2-methyl-1-imidazolyl)-5-[3-(2-methylphenoxyl)propyl]oxazole), compounds described in WO2004/039365), PKC inhibitors (e.g., ruboxistaurin mesylate), AGE inhibitors (e.g., ALT946, N-phenacylthiazolium bromide (ALT766), EXO-226, pyridorin, pyridoxamine), serotonin and noradrenalin reuptake inhibitors (e.g., duloxetine), sodium channel inhibitors (e.g., lacosamide), active oxygen scavengers (e.g., thioctic acid), cerebral vasodilators (e.g., tiapuride, mexiletine), somatostatin receptor agonists (e.g., BIM23190), and apoptosis signal regulating kinase-1 (ASK-1) inhibitors.

In some embodiments, the one or more additional therapeutic agents include those useful, for example, for treating hyperlipidemia. Non-limiting examples include HMG-COA reductase inhibitors (e.g., pravastatin, simvastatin, lovastatin, atorvastatin, fluvastatin, rosuvastatin, pitavastatin or a salt thereof (e.g., sodium salt, calcium salt)), squalene synthase inhibitors (e.g., compounds described in WO97/10224, e.g., N-[[(3R,5S)-1-(3-acetoxy-2,2-dimethylpropyl)-7-chloro-5-(2,3-dimethoxyphenyl)-2-oxo-1,2,3,5-tetrahydro-4, 1-benzoxazepin-3-yl]acetyl]piperidin-4-acetic acid), fibrate compounds (e.g., bezafibrate, clofibrate, simfibrate, clinofibrate), anion exchange resin (e.g., colestyramine), nicotinic acid drugs (e.g., nicomol, niceritrol, niaspan), phytosterols (e.g., soysterol, gamma oryzanol (γ-oryzanol)), cholesterol absorption inhibitors (e.g., zechia), CETP inhibitors (e.g., dalcetrapib, anacetrapib) and ω-3 fatty acid preparations (e.g., ω-3-fatty acid ethyl esters 90).

In some embodiments, the one or more additional therapeutic agents include those useful, for example, as anti-hypertensive agents. Non-limiting examples include angiotensin converting enzyme inhibitors (e.g., captopril, enalapril, delapril), angiotensin II antagonists (e.g., candesartan cilexetil, candesartan, losartan, losartan potassium, eprosartan, valsartan, telmisartan, irbesartan, tasosartan, olmesartan, olmesartan medoxomil, azilsartan, azilsartan medoxomil), calcium antagonists (e.g., manidipine, nifedipine, amlodipine, efonidipine, nicardipine, cilnidipine) and β-blockers (e.g., metoprolol, atenolol, propranolol, carvedilol, pindolol).

In some embodiments, the one or more additional therapeutic agents include those useful, for example, as diuretics. Non-limiting examples include_xanthine derivatives (e.g., theobromine sodium salicylate, theobromine calcium salicylate), thiazide preparations (e.g., ethiazide, cyclopenthiazide, trichloromethiazide, hydrochlorothiazide, hydroflumethiazide, benzylhydrochlorothiazide, penfluthiazide, polythiazide, methyclothiazide), antialdosterone preparations (e.g., spironolactone, triamterene), carbonic anhydrase inhibitors (e.g., acetazolamide) and chlorobenzenesulfonamide agents (e.g., chlortalidone, mefruside, indapamide).

In some embodiments, the one or more additional therapeutic agents include those useful, for example, as immunotherapeutic agents. Non-limiting examples include microbial or bacterial compounds (e.g., muramyl dipeptide derivative, picibanil), polysaccharides having immunoenhancing activity (e.g., lentinan, sizofiran, krestin), cytokines obtained by genetic engineering approaches (e.g., interferon, interleukin (IL) such as IL-1, IL-2, IL-12), and colony-stimulating factors (e.g., granulocyte colony-stimulating factor, erythropoietin).

In some embodiments, the one or more additional therapeutic agents include those useful, for example, as anti-thrombotic agents. Non-limiting examples include heparins (e.g., heparin sodium, heparin calcium, enoxaparin sodium, dalteparin sodium) warfarin (e.g., warfarin potassium); anti-thrombin drugs (e.g., aragatroban, dabigatran) FXa inhibitors (e.g., rivaroxaban, apixaban, edoxaban, YM150, compounds described in WO02/06234, WO2004/048363, WO2005/030740, WO2005/058823, and WO2005/113504) thrombolytic agents (e.g., urokinase, tisokinase, alteplase, nateplase, monteplase, pamiteplase), and platelet aggregation inhibitors (e.g., ticlopidine hydrochloride, clopidogrel, prasugrel, E5555, SHC530348, cilostazol, ethyl icosapentate, beraprost sodium, and sarpogrelate hydrochloride).

In some embodiments, the one or more additional therapeutic agents include those useful, for example, for treating osteoporosis. Non-limiting examples include alfacalcidol, calcitriol, elcatonin, calcitonin salmon, estriol, ipriflavone, pamidronate disodium, alendronate sodium hydrate, incadronate disodium, and risedronate disodium. Suitable examples of vitamins include vitamin B¹ and vitamin B¹². Suitable examples of erectile dysfunction drugs include apomorphine and sildenafil citrate. Suitable examples of therapeutic agents for urinary frequency or urinary incontinence include flavorxate hydrochloride, oxybutynin hydrochloride and propiverine hydrochloride. Suitable examples of therapeutic agents for dysuria include acetylcholine esterase inhibitors (e.g., distigmine). Suitable examples of anti-inflammatory agents include nonsteroidal anti-inflammatory drugs such as aspirin, acetaminophen, indomethacin.

Other exemplary additional therapeutic agents include agents that modulate hepatic glucose balance (e.g., fructose 1,6-bisphosphatase inhibitors, glycogen phosphorylase inhibitors, glycogen synthase kinase inhibitors, glucokinase activators), agents designed to treat the complications of prolonged hyperglycemia, such as aldose reductase inhibitors (e.g. epalrestat and ranirestat), agents used to treat complications related to micro-angiopathies, anti-dyslipidemia agents, such as HMG-CoA reductase inhibitors (statins, e.g. rosuvastatin), cholesterol-lowering agents, bile acid sequestrants (e.g., cholestyramine), cholesterol absorption inhibitors (e.g. plant sterols such as phytosterols), cholesteryl ester transfer protein (CETP) inhibitors, inhibitors of the ileal bile acid transport system (IBAT inhibitors), bile acid binding resins, nicotinic acid (niacin) and analogues thereof, anti-oxidants (e.g., probucol), omega-3 fatty acids, antihypertensive agents, including adrenergic receptor antagonists, such as beta blockers (e.g. atenolol), alpha blockers (e.g. doxazosin), and mixed alpha/beta blockers (e.g. labetalol), adrenergic receptor agonists, including alpha-2 agonists (e.g. clonidine), angiotensin converting enzyme (ACE) inhibitors (e.g. lisinopril), calcium channel blockers, such as dihydropridines (e.g. nifedipine), phenylalkylamines (e.g. verapamil), and benzothiazepines (e.g. diltiazem), angiotensin II receptor antagonists (e.g. candesartan), aldosterone receptor antagonists (e.g. eplerenone), centrally acting adrenergic drugs, such as central alpha agonists (e.g. clonidine), diuretic agents (e.g. furosemide), haemostasis modulators, including antithrombotics (e.g., activators of fibrinolysis), thrombin antagonists, factor VIIa inhibitors, anticoagulants (e.g., vitamin K antagonists such as warfarin), heparin and low molecular weight analogues thereof, factor Xa inhibitors, and direct thrombin inhibitors (e.g. argatroban), antiplatelet agents (e.g., cyclooxygenase inhibitors (e.g. aspirin)), adenosine diphosphate (ADP) receptor inhibitors (e.g. clopidogrel), phosphodiesterase inhibitors (e.g. cilostazol), glycoprotein IIB/IIA inhibitors (e.g. tirofiban), adenosine reuptake inhibitors (e.g. dipyridamole), noradrenergic agents (e.g. phentermine), serotonergic agents (e.g. sibutramine), diacyl glycerolacyltransferase (DGAT) inhibitors, feeding behavior modifying agents, pyruvate dehydrogenase kinase (PDK) modulators, serotonin receptor modulators, monoamine transmission-modulating agents, such as selective serotonin reuptake inhibitors (SSRI) (e.g. fluoxetine), noradrenaline reuptake inhibitors (NARI), noradrenaline-serotonin reuptake inhibitors (SNRI), and monoamine oxidase inhibitors (MAOI) (e.g. toloxatone and amiflamine), compounds described in W^{0007/013694}, WO2007/018314, WO2008/093639 and WO2008/099794, GPR40 agonists (e.g., fasiglifam or a hydrate thereof, compounds described in WO2004/041266, WO2004/106276, WO2005/063729, WO2005/063725, WO2005/087710, WO2005/095338, WO2007/013689 and WO2008/001931), SGLT1 inhibitors, adiponectin or agonist thereof, IKK inhibitors (e.g., AS-2868), somatostatin receptor agonists, ACC2 inhibitors, cachexia-ameliorating agents, such as a cyclooxygenase inhibitors (e.g., indomethacin), progesterone derivatives (e.g., megestrol acetate), glucocorticoids (e.g., dexamethasone), metoclopramide agents, tetrahydrocannabinol agents, agents for improving fat metabolism (e.g., eicosapentaenoic acid), growth hormones, IGF-1, antibodies against a cachexia-inducing factor TNF-α, LIF, IL-6, and oncostatin M, metabolism-modifying proteins or peptides such as glucokinase (GK), glucokinase regulatory protein (GKRP), uncoupling proteins 2 and 3 (UCP2 and UCP3), peroxisome proliferator-activated receptor α (PPARα), MC4r agonists, insulin receptor agonist, PDE 5 inhibitors, glycation inhibitors (e.g., ALT-711), nerve regeneration-promoting drugs (e.g., Y-128, VX853, prosaptide), antidepressants (e.g., desipramine, amitriptyline, imipramine), antiepileptic drugs (e.g., lamotrigine, trileptal, keppra, zonegran, pregabalin, harkoseride, carbamazepine), antiarrhythmic drugs (e.g., mexiletine), acetylcholine receptor ligands (e.g., ABT-594), endothelin receptor antagonists (e.g., ABT-627), narcotic analgesics (e.g., morphine), α2 receptor agonists (e.g., clonidine), local analgesics (e.g., capsaicin), antianxiety drugs (e.g., benzothiazepine), phosphodiesterase inhibitors (e.g., sildenafil), dopamine receptor agonists (e.g., apomorphine), cytotoxic antibodies (e.g., T-cell receptor and IL-2 receptor-specific antibodies), B cell depleting therapies (e.g., anti-CD20 antibody (e.g., rituxan), i-BLyS antibody), drugs affecting T cell migration (e.g., anti-integrin alpha 4/beta 1 antibody (e.g., tysabri), drugs that act on immunophilins (e.g., cyclosporine, tacrolimus, sirolimus, rapamicin), interferons (e.g., IFN-β), immunomodulators (e.g., glatiramer), TNF-binding proteins (e.g., circulating receptors), immunosupressants (e.g., mycophenolate), and metaglidasen, AMG-131, balaglitazone, MBX-2044, rivoglitazone, aleglitazar, chiglitazar, lobeglitazone, PLX-204, PN-2034, GFT-505, THR-0921, exenatide, exendin-4, memantine, midazolam, ketoconazole, ethyl icosapentate, clonidine, azosemide, isosorbide, ethacrynic acid, piretanide, bumetanide, etoposide.

In some embodiments, the one or more additional therapeutic agents include those useful, for example, as anti-emetic agents. As used herein, an “anti-emetic” agent refers to any agent that counteracts (e.g., reduces or removes) nausea or emesis (vomiting). While not wishing to be bound by theory, it is believed that administering one or more anti-emetic agents in combination with the formula (I) compounds described herein may allow higher dosages of the formula (I) compounds to be administered, e.g., because the patient may be able to have a normal food intake and thereby respond faster to the treatment.

Non-limiting examples of anti-emetic agents include 5HT3-receptor antagonists (serotonin receptor antagonists), neuroleptics/anti-psychotics, antihistamines, anticholinergic agents, steroids (e.g., corticosteroids), NK1-receptor antagonists (e.g., Neurokinin 1 substance P receptor antagonists), antidopaminergic agents/dopamine receptor antagonists, benzodiazepines, cannabinoids.

For example, the antiemetic agent can be selected from the group consisting of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In some embodiments, the anti-emetic agent is a 5HT3-receptor antagonist (serotonin receptor antagonist). Non-limiting examples of 5HT3-receptor antagonists (serotonin receptor antagonists) include: Granisetron (Kytril), Dolasetron, Ondansetron (Zofran), Tropisetron, Ramosetron, Palonosetron, Alosetron, azasetron, Bemesetron, Zatisetron, Batanopirde, MDL-73147EF; Metoclopramide, N-3389 (endo-3,9-dimethyl-3,9-diazabicyclo[3,3,1]non-7-yl-1H-indazole-3-carboxamide dihydrochloride), Y-25130 hydrochloride, MDL 72222, Tropanyl-3,5-dimethylbenzoate, 3-(4-Allylpiperazin-1-yl)-2-quinoxalinecarbonitrile maleate, Zacopride hydrochloride, and Mirtazepine. Other non-limiting examples of 5HT3-receptor antagonists (serotonin receptor antagonists) include: cilansetron, clozapine, cyproheptadine, dazopride, hydroxyzine, lerisetron, metoclopramide, mianserin, olanzapine, palonosetron (+netupitant), quetiapine, qamosetron, ramosteron, ricasetron, risperidone, ziprasidone, and zatosetron.

In certain embodiments, the 5HT-3-receptor antagonist is Granisetron, Dolasetron, Ondansetron hydrochloride, Tropisetron, Ramosetron, Palonosetron, Alosetron, Bemesetron, Zatisetron, Batanopirde, MDL-73147EF, Metoclopramide, N-3389, Y-25130 hydrochloride, MDL 72222, Tropanyl-3,5-dimethylbenzoate 3-(4-AIIyI-piperazin-1-yl)-2-quinoxalinecarbonitrile maleate, Zacopride hydrochloride and Mirtazepine.

In certain embodiments, the 5HT-3-receptor antagonist is Granisetron, Dolasetron, Ondansetron hydrochloride, Tropisetron, Ramosetron, Palonosetron, Alosetron, Bemesetron, and Zatisetron.

In certain embodiments, the 5HT-3-receptor antagonist is Granisetron, Dolasetron and Ondansetron.

In certain embodiments, the 5HT-3-receptor antagonist is Granisetron.

In certain embodiments, the 5HT-3-receptor antagonist is Ondansetron.

In some embodiments, the anti-emetic agent is an antihistamine. Non-limiting examples of antihistamines include: piperazine derivatives (e.g., cyclizine, meclizine, and cinnarizine); Promethazine; Dimenhydrinate (Dramamine, Gravol); Diphenhydramine; Hydroxyzine; Buclizine; and Meclizine hydrochloride (Bonine, Antivert), doxylamine, and mirtazapine.

In some embodiments, the anti-emetic agent is an anticholinergic agent (Inhibitors of the acetylcholine receptors). Non-limiting examples of anticholinergic agents include: atropine, Scopolamine, Glycopyrron, Hyoscine, Artane (Trihexy-5 trihexyphenidyl hydrochloride), Cogentin (benztropine mesylate), Akineton (biperiden hydrochloride), Disipal (Norflex orphenadrine citrate), diphenhydramine, hydroxyzine, hyoscyamine, and Kemadrin (procyclidine hydrochloride).

In some embodiments, the anti-emetic agent is a steroid (e.g., a corticosteroid). Non-limiting examples of steroids include: betamethasone, Dexamethasone, Methylprednisolone, Prednisone®, and Trimethobenzamide (Tigan).

In some embodiments, the anti-emetic agent is an NK1-receptor antagonists (e.g., Neurokinin 1 substance P receptor antagonists). Non-limiting examples of NK1-receptor antagonists include: aprepitant, casopitant, ezlopitant, fosaprepitant, maropitant, netupitant, rolapitant, and vestipitant.

Other non-limiting examples of NK1-receptor antagonists include: MPC-4505, GW597599, MPC-4505, GR205171, L-759274, SR 140333, CP-96,345, BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-1, CJ-11974 j. Benserazide and carbidopa k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g] [1,7]naphthyridine-6,13-dione], PD 154075, ([(2-benzofuran)-CH2OCO]—(R)-alpha-MeTrp-(S)—NHCH(CH3) Ph), FK888, and (D-Pro4, D-Trp7,9,10, Phe11)SP4-11.

In some embodiments, the anti-emetic agent is an anti-dopaminergic agents/dopamine receptor antagonist (e.g., dopamine receptor antagonist, e.g., D2 or D3 antagonists). Non-limiting examples include phenothiazines (e.g., promethazine, chlorpromazine, prochlorperazine, perphenazine, hydroxyzine, thiethylperazine, metopimazine); benzamides (e.g., Metoclopramide, domperidone), butyrophenones (e.g., haloperidol, droperidol); alizapride, bromopride, clebopride, domperidone, itopride, metoclopramide, trimethobenzamide, and amisulpride.

In some embodiments, the anti-emetic agent is a non-psychoactive cannabinoids (e.g., Cannabidiol (CBD), Cannabidiol dimethylheptyl (CBD-DMH), Tetra-hydro-cannabinol (THC), Cannabinoid agonists such as WIN 55-212 (a CB1 and CB2 receptor agonist), Dronabinol (Marinol®), and Nabilone (Cesamet)).

Other exemplary anti-emetic agents include: c-9280 (Merck); benzodiazepines (diazepam, midazolam, lorazepam); neuroleptics/anti-psychotics (e.g., dixyrazine, haloperidol, and Prochlorperazine (Compazine®)); cerium oxalate; propofol; sodium citrate; dextrose; fructose (Nauzene); orthophosphoric acid; fructose; glucose (Emetrol); bismuth subsalicylate (Pepto Bismol); ephedrine; vitamin B⁶; peppermint, lavender, and lemon essential oils; and ginger.

Still other exemplary anti-emetic agents include those disclosed in US 20120101089A1; U.S. Pat. No. 10,071,088 B²; U.S. Pat. No. 6,673,792 B¹; U.S. Pat. No. 6,197,329 B¹; U.S. Pat. No. 10,828,297 B²; U.S. Pat. No. 10,322,106 B²; U.S. Pat. No. 10,525,033 B²; WO 2009080351 A1; WO 2019203753 A2; WO 2002020001 A2; U.S. Pat. No. 8,119,697 B²; U.S. Pat. No. 5,039,528; US20090305964A1; and WO 2006/111169, each of which is incorporated by reference in its entirety.

In some embodiments, the additional therapeutic agent or regimen is administered to the patient prior to contacting with or administering the compounds and pharmaceutical compositions (e.g., about one hour prior, or about 6 hours prior, or about 12 hours prior, or about 24 hours prior, or about 48 hours prior, or about 1 week prior, or about 1 month prior).

In some embodiments, the additional therapeutic agent or regimen is administered to the patient at about the same time as contacting with or administering the compounds and pharmaceutical compositions. By way of example, the additional therapeutic agent or regimen and the compounds and pharmaceutical compositions are provided to the patient simultaneously in the same dosage form. As another example, the additional therapeutic agent or regimen and the compounds and pharmaceutical compositions are provided to the patient concurrently in separate dosage forms.

Patient Selection

In some embodiments, the methods described herein further include the step of identifying a patient (e.g., a subject) in need of such treatment (e.g., by way of blood assay, body mass index, or other conventional method known in the art).

In some embodiments, the methods described herein further include the step of identifying a patient (e.g., patient) that has a disease, disorder, or condition as provided here (e.g., a GLP-1 associated disease, disorder, or condition).

In some embodiments, the methods described herein further include the step of identifying a patient (e.g., patient) that has type 2 diabetes mellitus. In some embodiments, determining if the patient has type 2 diabetes mellitus includes performing an assay to determine the level of hemoglobin A1c (HbA1c), fasting plasma glucose, non-fasting plasma glucose, or any combination thereof. In some embodiments, the level of HbA1c is about 6.5% to about 24.0%. In some embodiments, the level of HbA1c is greater than or about 6.5%. In some embodiments, the level of HbA1c is greater than or about 8.0%. In some embodiments, the level of HbA1c is greater than or about 10.0%. In some embodiments, the level of HbA1c is greater than or about 12.0%. In some embodiments, the level of HbA1c is greater than or about 14.0%. In some embodiments, the level of HbA1c is greater than or about 16.0%. In some embodiments, the level of HbA1c is greater than or about 18.0%. In some embodiments, the level of HbA1c is greater than or about 20.0%. In some embodiments, the level of HbA1c is greater than or about 22.0%. In some embodiments, the level of HbA1c is greater than or about 24.0%.

In some embodiments, the level of fasting plasma glucose is greater than or about 120 mg/dL to greater than or about 750 mg/dL. In some embodiments, the level of fasting plasma glucose is greater than or about 200 mg/dL to greater than or about 500 mg/dL. In some embodiments, the level of fasting plasma glucose is greater than or about 300 mg/dL to greater than or about 700 mg/dL.

In some embodiments, the level of non-fasting plasma glucose is greater than or about 190 mg/dL to greater than or about 750 mg/dL. In some embodiments, the level of non-fasting plasma glucose is greater than or about 250 mg/dL to greater than or about 450 mg/dL. In some embodiments, the level of non-fasting plasma glucose is greater than or about 400 mg/dL to greater than or about 700 mg/dL.

In some embodiments, determining if the patient has type 2 diabetes mellitus further includes determining the patient's BMI. In some embodiments, the BMI of the patient is greater than or about 22 kg/m² to greater than or about 100 kg/m². In some embodiments, the BMI of the patient is greater than or about 30 kg/m² to greater than or about 90 kg/m². In some embodiments, the BMI of the patient is greater than or about 40 kg/m² to greater than or about 80 kg/m². In some embodiments, the BMI of the patient is greater than or about 50 kg/m² to greater than or about 70 kg/m².

In some embodiments, additional factors (e.g. risk factors) used for determining if the patient has type 2 diabetes mellitus further includes age and ethnicity of the patient. In some embodiments, the patient's age is greater than or about 10 years. In some embodiments, the patient's age is greater than or about 15 years. In some embodiments, the patient's age is greater than or about 20 years. In some embodiments, the patient's age is greater than or about 25 years. In some embodiments, the patient's age is greater than or about 30 years. In some embodiments, the patient's age is greater than or about 35 years. In some embodiments, the patient's age is greater than or about 40 years. In some embodiments, the patient's age is greater than or about 42 years. In some embodiments, the patient's age is greater than or about 44 years. In some embodiments, the patient's age is greater than or about 46 years. In some embodiments, the patient's age is greater than or about 48 years. In some embodiments, the patient's age is greater than or about 50 years. In some embodiments, the patient's age is greater than or about 52 years. In some embodiments, the patient's age is greater than or about 54 years. In some embodiments, the patient's age is greater than or about 56 years. In some embodiments, the patient's age is greater than or about 58 years. In some embodiments, the patient's age is greater than or about 60 years. In some embodiments, the patient's age is greater than or about 62 years. In some embodiments, the patient's age is greater than or about 64 years. In some embodiments, the patient's age is greater than or about 66 years. In some embodiments, the patient's age is greater than or about 68 years. In some embodiments, the patient's age is greater than or about 70 years. In some embodiments, the patient's age is greater than or about 72 years. In some embodiments, the patient's age is greater than or about 74 years. In some embodiments, the patient's age is greater than or about 76 years. In some embodiments, the patient's age is greater than or about 78 years. In some embodiments, the patient's age is greater than or about 80 years. In some embodiments, the patient's age is greater than or about 85 years. In some embodiments, the patient's age is greater than or about 90 years. In some embodiments, the patient's age is greater than or about 95 years. In some embodiments, the ethnicity of the patient may be African American, American Indian or Alaska Native, Asian American, Hispanics or Latinos, or Native Hawaiian or Pacific Islander.

### EXAMPLES

The invention is further described in the following examples, which do not limit the scope of the invention described in the claims.

General information: All evaporations were carried out in vacuo with a rotary evaporator. Analytical samples were dried in vacuo (1-5 mmHg) at rt. Thin layer chromatography (TLC) was performed on silica gel plates, spots were visualized by UV light (214 and 254 nm). Purification by column and flash chromatography was carried out using silica gel (100-200 mesh). Solvent systems were reported as mixtures by volume. NMR spectra were recorded on a Bruker 400 or Varian (400 MHz) spectrometer. ¹H chemical shifts are reported in δ values in ppm with the deuterated solvent as the internal standard. Data are reported as follows: chemical shift, multiplicity (s=singlet, d=doublet, t=triplet, q=quartet, br=broad, m=multiplet), coupling constant (Hz), integration. LCMS spectra were obtained on SHIMADZU LC20-MS2020 or Agilent 1260 series 6125B mass spectrometer or Agilent 1200 series, 6110 or 6120 mass spectrometer with electrospray ionization and excepted as otherwise indicated.

### Example 1: N-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)-5-methylpyridine-3-carboxamide (Compound 101a)

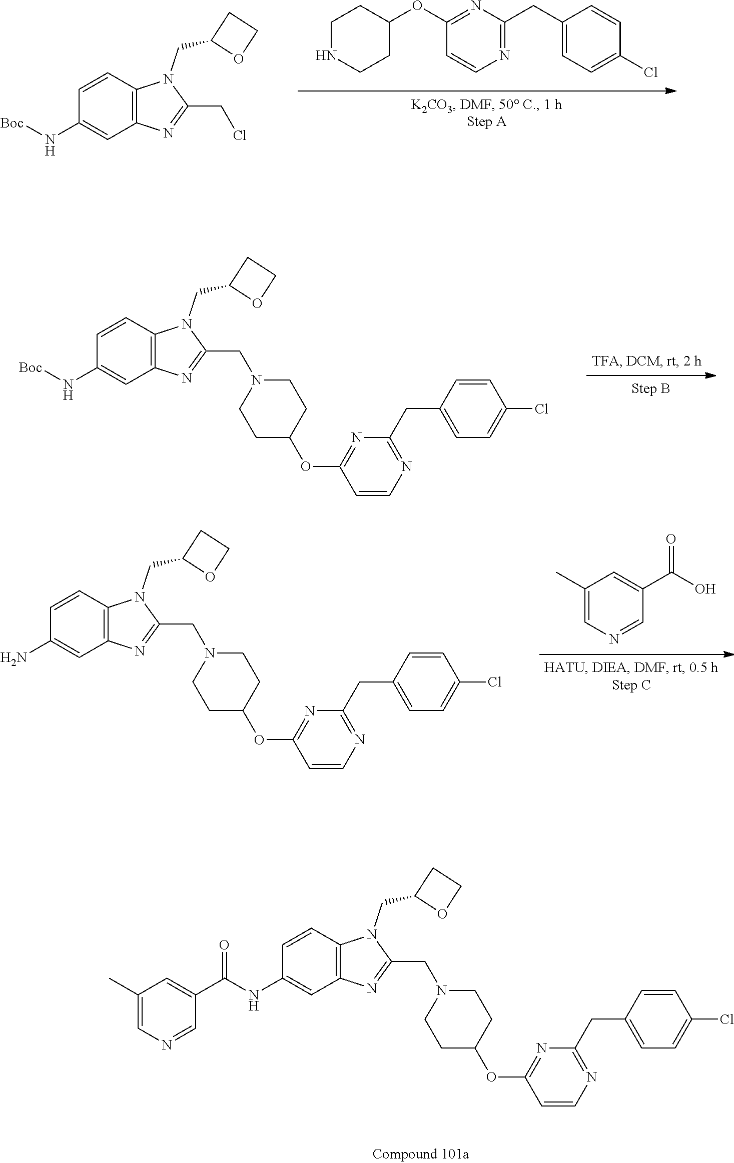

### Step A: tert-butyl (S)-(2-((4-((2-(4-chlorobenzyl)pyrimidin-4-yl)oxy)piperidin-1-yl)methyl)-1-(oxetan-2-ylmethyl)-1H-benzo[d]imidazol-5-yl)carbamate

To a solution of tert-butyl (S)-(2-(chloromethyl)-1-(oxetan-2-ylmethyl)-1H-benzo[d]imidazol-5-yl)carbamate (5.32 g, 15.15 mmol) in DMF (40 mL) was added K₂CO₃ (6.27 g, 45.45 mmol) and 2-(4-chlorobenzyl)-4-(piperidin-4-yloxy)pyrimidine (4.6 g, 15.15 mmol). The resulting mixture was stirred at 50° C. for 1 hours. After the reaction was completed, the reaction mixture was filtered and the filtrate concentrated. The crude product was purified by column chromatography on silica gel (DCM:MeOH=80:1) to give tert-butyl (S)-(2-((4-((2-(4-chlorobenzyl)pyrimidin-4-yl)oxy)piperidin-1-yl)methyl)-1-(oxetan-2-ylmethyl)-1H-benzo[d]imidazol-5-yl)carbamate (2.33 g, yield: 78%) as brown oil.

MS Calcd.: 618.3; MS Found: 619.3 [M+H]⁺.

### Step B: (S)-2-((4-((2-(4-chlorobenzyl)pyrimidin-4-yl)oxy)piperidin-1-yl)methyl)-1-(oxetan-2-ylmethyl)-1H-benzo[d]imidazol-5-amine

To a solution of tert-butyl (S)-(2-((4-((2-(4-chlorobenzyl)pyrimidin-4-yl)oxy)piperidin-1-yl)methyl)-1-(oxetan-2-ylmethyl)-1H-benzo[d]imidazol-5-yl)carbamate (1.64 g, 2.65 mmol) in DCM (15 mL) was added TFA (3 mL). The solution was stirred at room temperature for 2 hours. The reaction mixture was concentrated under vacuum to give (S)-2-((4-((2-(4-chlorobenzyl)pyrimidin-4-yl)oxy)piperidin-1-yl)methyl)-1-(oxetan-2-ylmethyl)-1H-benzo[d]imidazol-5-amine (1.04 g) as brown oil.

MS Calcd.: 518.2; MS Found: 519.2 [M+H]⁺.

### Step C: N-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)-5-methylpyridine-3-carboxamide (Compound 101a)

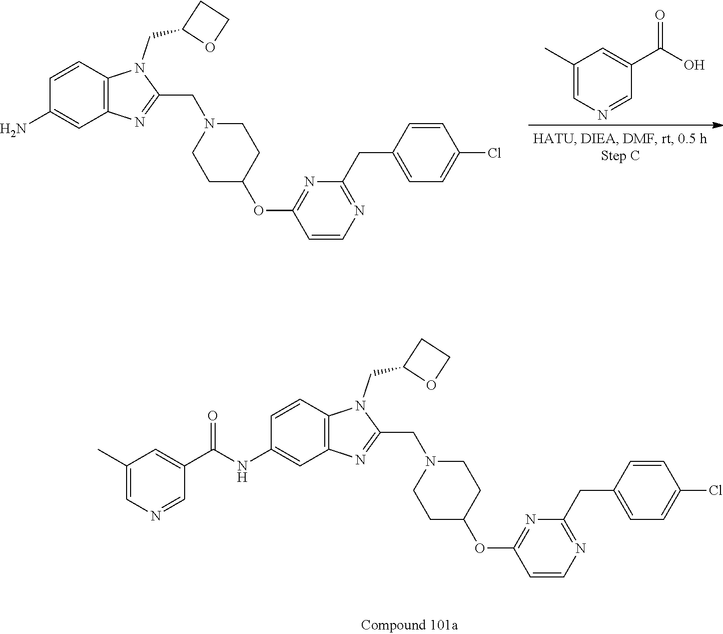

To a solution of 5-methylnicotinic acid (64 mg, 0.47 mmol) in DMF (2 ml) was added DIEA (151 mg, 1.17 mmol), HATU (194 mg, 0.51 mmol) and (S)-2-((4-((2-(4-chlorobenzyl)pyrimidin-4-yl)oxy)piperidin-1-yl)methyl)-1-(oxetan-2-ylmethyl)-1H-benzo[d]imidazol-5-amine (200 mg, 0.39 mmol) was added. The resulting mixture was stirred at room temperature for 0.5 h. After the reaction was completed, the reaction mixture was purified directly by Prep-HPLC to give Compound 101a (7.68 mg, yield: 3%) as a white solid.

MS Calcd.: 637.3; MS Found: 638.3 [M+H]⁺.

¹H NMR (400 MHz, DMSO-d₆): 10.36 (s, 1H), 8.93 (d, J=2.0 Hz, 1H), 8.60 (d, J=1.2 Hz, 1H), 8.41 (d, J=5.6 Hz, 1H), 8.12-8.15 (m, 1H), 8.04 (d, J=1.6 Hz, 1H), 7.53-7.61 (m, 2H), 7.31-7.36 (m, 4H), 6.73 (d, J=6.0 Hz, 1H), 4.98-5.12 (m, 2H), 4.66-4.71 (m, 1H), 4.47-4.58 (m, 2H), 4.35-4.40 (m, 1H), 4.08 (s, 2H), 3.89 (d, J=13.6 Hz, 1H), 3.77 (d, J=13.6 Hz, 1H), 2.65-2.79 (m, 3H), 2.30-2.47 (m, 6H), 1.88-1.96 (m, 2H), 1.58-1.67 (m, 2H).

### Example 2: N-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)pyridine-3-carboxamide (Compound 102a)

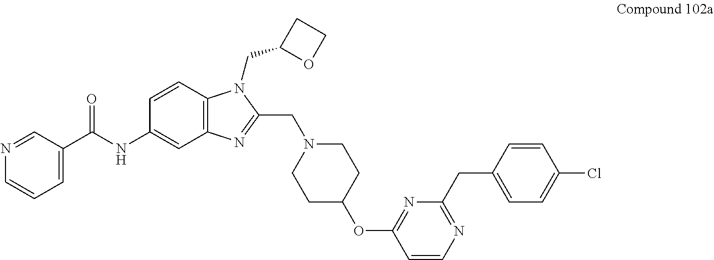 [SMILES: O=C(Nc1ccc2c(c1)nc(CN1CCC(Oc3ccnc(Cc4ccc(Cl)cc4)n3)CC1)n2C[C@@H]1CCO1)c1cccnc1]

The synthesis of Compound 102a was similar to that of Compound 101a except that nicotinic acid was used instead of 5-methylnicotinic acid. Compound 102a (5.75 mg, yield: 2%) was obtained as a white solid.

MS Calcd.: 623.2; MS Found: 624.3 [M+H]⁺.

¹H NMR (400 MHz, DMSO-d₆): 10.61 (s, 1H), 8.76 (d, J=4.4 Hz, 1H), 8.41 (d, J=6.0 Hz, 1H), 8.18-8.20 (m, 2H), 8.06-8.10 (m, 1H), 7.67-7.72 (m, 2H), 7.59 (d, J=8.8 Hz, 1H), 7.32-7.37 (m, 4H), 6.74 (d, J=5.6 Hz, 1H), 4.99-5.13 (m, 2H), 4.66-4.72 (m, 1H), 4.47-4.59 (m, 2H), 4.36-4.41 (m, 1H), 4.09 (s, 2H), 3.90 (d, J=13.6 Hz, 1H), 3.78 (d, J=13.6 Hz, 1H), 2.66-2.80 (m, 3H), 2.30-2.52 (m, 3H), 1.89-1.96 (m, 2H), 1.58-1.78 (m, 2H).

### Example 3: N-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)-2-methylpyridine-3-carboxamide (Compound 103a)

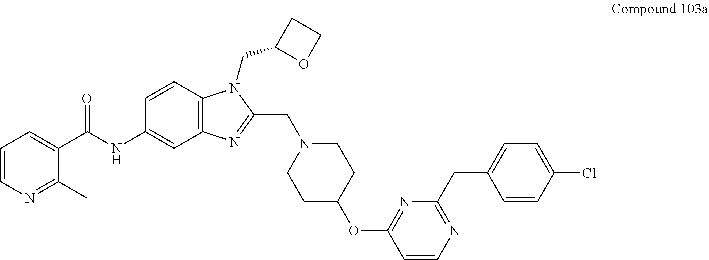 [SMILES: Cc1ncccc1C(=O)Nc1ccc2c(c1)nc(CN1CCC(Oc3ccnc(Cc4ccc(Cl)cc4)n3)CC1)n2C[C@@H]1CCO1]

The synthesis of Compound 103a was similar to that of Compound 101a except that 2-methylnicotinic acid was used instead of 5-methylnicotinic acid. Compound 103a (10.14 mg, yield: 4%) as obtained as a white solid.

¹H NMR (400 MHz, DMSO-d₆): 10.38 (s, 1H), 8.55 (dd, J=4.8 Hz, 1.6 Hz, 1H), 8.41 (d, J=6.0 Hz, 1H), 8.03 (d, J=1.6 Hz, 1H), 7.88 (dd, J=7.6 Hz, 1.6 Hz, 1H), 7.58 (d, J=8.4 Hz, 1H), 7.50 (dd, J=8.8 Hz, 1.6 Hz, 1H), 7.31-7.40 (m, 5H), 6.73 (d, J=5.6 Hz, 1H), 4.95-5.12 (m, 2H), 4.63-4.72 (m, 1H), 4.46-4.60 (m, 2H), 4.33-4.38 (m, 1H), 4.08 (s, 2H), 3.88 (d, J=13.6 Hz, 1H), 3.77 (d, J=13.2 Hz, 1H), 2.64-2.80 (m, 3H), 2.58 (s, 3H), 2.30-2.45 (m, 3H), 1.89-1.98 (m, 2H), 1.55-1.70 (m, 2H).

### Example 4: N-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)-6-methylpyridine-3-carboxamide (Compound 104a)

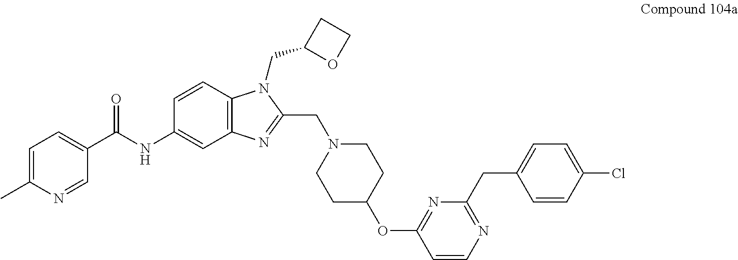 [SMILES: Cc1ccc(C(=O)Nc2ccc3c(c2)nc(CN2CCC(Oc4ccnc(Cc5ccc(Cl)cc5)n4)CC2)n3C[C@@H]2CCO2)cn1]

The synthesis of Compound 104a was similar to that of Compound 101a except that 6-methylnicotinic acid was used instead of 5-methylnicotinic acid. Compound 104a (13.26 mg, yield: 5%) as obtained as a white solid.

MS Calcd.: 637.3; MS Found: 638.3 [M+H]⁺.

¹H NMR (400 MHz, DMSO-d₆): 10.44 (s, 1H), 8.57 (s, 1H), 8.54 (d, J=4.8 Hz, 1H), 8.41 (d, J=5.6 Hz, 1H), 8.03 (s, 1H), 7.59 (d, J=7.2 Hz, 1H), 7.46-7.52 (m, 2H), 7.30-7.37 (m, 4H), 6.73 (d, J=5.6 Hz, 1H), 4.97-5.12 (m, 2H), 4.65-4.71 (m, 1H), 4.45-4.58 (m, 2H), 4.33-4.39 (m, 1H), 4.09 (s, 2H), 3.88 (d, J=13.2 Hz, 1H), 3.78 (d, J=13.6 Hz, 1H), 2.67-2.78 (m, 3H), 2.30-2.45 (m, 6H), 1.89-1.98 (m, 2H), 1.56-1.70 (m, 2H).

### Example 5: N-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)-4-methylpyridine-3-carboxamide (Compound 105a)

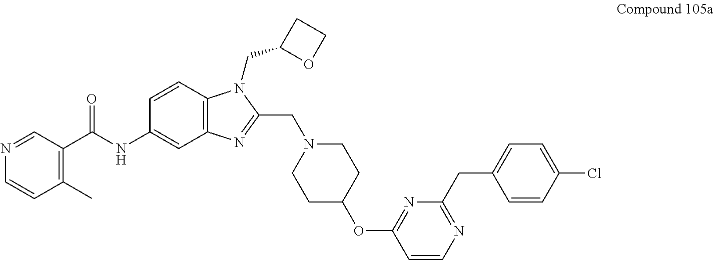 [SMILES: Cc1ccncc1C(=O)Nc1ccc2c(c1)nc(CN1CCC(Oc3ccnc(Cc4ccc(Cl)cc4)n3)CC1)n2C[C@@H]1CCO1]

The synthesis of Compound 105a was similar to that of Compound 101a except that 4-methylnicotinic acid was used instead of 5-methylnicotinic acid. Compound 105a (12.70 mg, yield: 8.6%) as obtained a white solid.

MS Calcd.: 637.3; MS Found: 638.3 [M+H]+.

¹H NMR (400 MHz, DMSO-d₆): 10.42 (s, 1H), 8.65 (s, 1H), 8.53 (d, J=4.8 Hz, 1H), 8.41 (d, J=6.0 Hz, 1H), 8.04 (d, J=2.0 Hz, 1H), 7.57-7.60 (m, 1H), 7.49-7.53 (m, 1H), 7.31-7.40 (m, 5H), 6.73 (d, J=5.6 Hz, 1H), 4.80-5.12 (m, 2H), 4.62-4.70 (m, 1H), 4.46-4.59 (m, 2H), 4.33-4.40 (m, 1H), 4.08 (s, 2H), 3.88 (d, J=13.2 Hz, 1H), 3.77 (d, J=13.2 Hz, 1H), 2.64-2.80 (m, 3H), 2.40-2.45 (m, 4H), 2.30-2.39 (m, 2H), 1.85-1.98 (m, 2H), 1.55-1.69 (m, 2H).

### Example 6: 2-{[4-({2-[(4-chlorophenyl)methyl]pyrimidin-4-yl}oxy)piperidin-1-yl]methyl}-1-{[(2S)-oxetan-2-yl]methyl}-5-[5-(trifluoromethyl)-4H-1,2,4-triazol-3-yl]-1H-1,3-benzodiazole (Compound 106a)

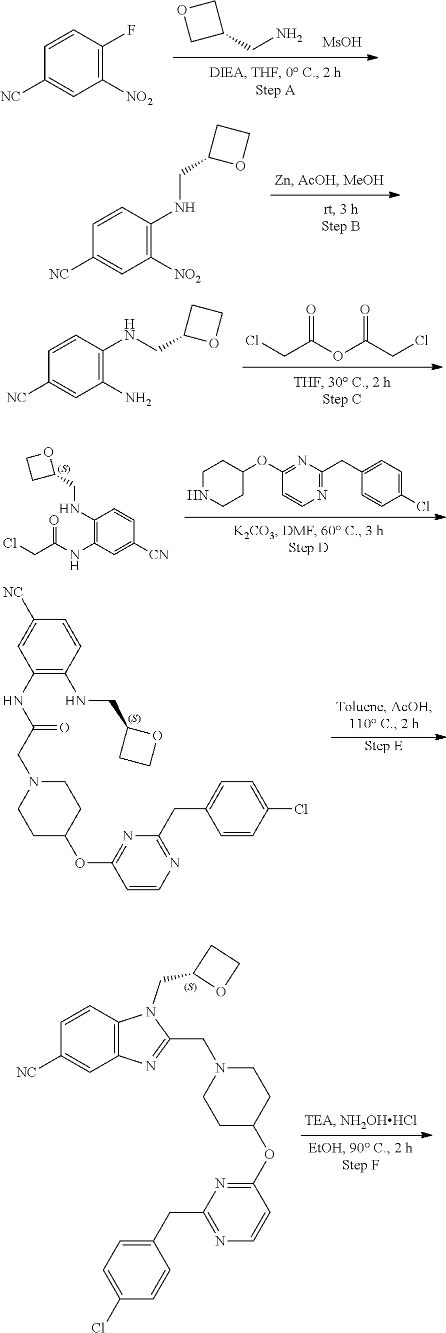

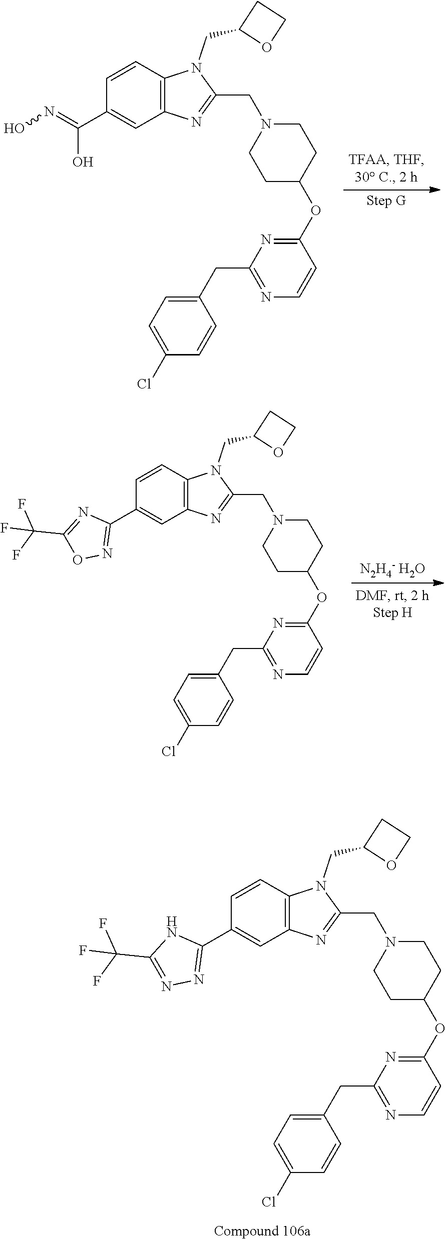

### Step A: (S)-3-nitro-4-((oxetan-2-ylmethyl)amino)benzonitrile

To a solution 4-fluoro-3-nitrobenzonitrile (2.5 g, 15.1 mmol) in THF (25 mL) was added (S)-oxetan-2-ylmethanamine (3.04 g, 16.61 mmol) and DIEA (5.84 g, 45.3 mmol) successively at 0° C. The reaction mixture was stirred at 0° C. for 2 hours. After the reaction was completed, the reaction was quenched with H₂O and extracted with EA (30 mL*3). The organic layers were combined, dried over Na₂SO₄ and concentrated. The residue was purified by silica gel column chromatography (PE/EA=1/1) to give (S)-3-nitro-4-((oxetan-2-ylmethyl)amino)benzonitrile (2.4 g, 80% yield) as a yellow solid.

MS Calcd.: 233.1; MS Found: 234.0 [M+H]⁺.

### Step B: (S)-3-amino-4-((oxetan-2-ylmethyl)amino)benzonitrile

To a solution of (S)-3-nitro-4-((oxetan-2-ylmethyl)amino)benzonitrile (2.4 g, 10.3 mmol) and Zn (6.7 g, 103 mmol) in MeOH (24 mL) was added AcOH (1.85 g) at room temperature. The reaction mixture was stirred at rt for 3 hours. After the reaction was completed, the reaction was filtered and the solid washed with NaHCO₃ (aq). The filtrate was quenched with H₂O and extracted with EA (20 mL*3). The organic layers were combined, dried over Na₂SO₄ and concentrated to give (S)-3-amino-4-((oxetan-2-ylmethyl)amino)benzonitrile (1.4 g, 58.3% yield) as yellow solid.

MS Calcd.: 203.1; MS Found: 204.1 [M+H]⁺.

### Step C: (S)-2-chloro-N-(5-cyano-2-((oxetan-2-ylmethyl)amino)phenyl) acetamide

To a solution of (S)-3-nitro-4-((oxetan-2-ylmethyl)amino)benzonitrile (1 g, 4.9 mmol) in THE (10 mL) was added 2-chloroacetic anhydride (1 g, 5.9 mmol). The reaction was stirred at 30° C. for 2 hours. After the reaction was completed, the residue was purified by silica gel column chromatography (DCM/MEOH=10/1) to give (S)-2-chloro-N-(5-cyano-2-((oxetan-2-ylmethyl)amino)phenyl)acetamide (230 mg, 23% yield) as a yellow solid.

MS Calcd.: 279.1; MS Found: 280.1 [M+H]⁺.

### Step D: (S)-2-(4-((2-(4-chlorobenzyl)pyrimidin-4-yl)oxy)piperidin-1-yl)-N-(5-cyano-2-((oxetan-2-ylmethyl)amino)phenyl)acetamide

A mixture of 2-(4-chlorobenzyl)-4-(piperidin-4-yloxy)pyrimidine (216 mg, 0.72 mmol) and K₂CO₃ (298 mg, 2.16 mmol) in DMF (2 ml) was added in (S)-2-chloro-N-(5-cyano-2-((oxetan-2-ylmethyl)amino)phenyl)acetamide (200 mg, 0.72 mmol). The reaction was stirred at 60° C. for 3 hours. After the reaction was completed, the reaction was quenched with H₂O and extracted with EA (5 mL*3). The organic layers were combined, dried over Na₂SO₄ and concentrated. The residue was purified by silica gel column chromatography (DCM/MeOH=20/1) to give (S)-2-(4-((2-(4-chlorobenzyl)pyrimidin-4-yl)oxy)piperidin-1-yl)-N-(5-cyano-2-((oxetan-2-ylmethyl)amino)phenyl)acetamide (250 mg, 60% yield) as yellow solid.

MS Calcd.: 546.2; MS Found: 547.5 [M+H]⁺

### Step E: (S)-2-((4-((2-(4-chlorobenzyl)pyrimidin-4-yl)oxy)piperidin-1-yl)methyl)-1-(oxetan-2-ylmethyl)-1H-benzo[d]imidazole-5-carbonitrile

To a solution of 2-(4-(2-(4-chloro-2-fluorophenyl)-2-methylbenzo[d][1,3]dioxol-4-yl)piperidin-1-yl)-N-(5-cyano-4-fluoro-2-((((S)-oxetan-2-yl)methyl) amino)phenyl)acetamide (250 mg, 0.46 mmol) in Toluene (3 ml) was added in AcOH (1 mL). The reaction was stirred at 110° C. for 2 hours. After the reaction was completed, the reaction was quenched with H₂O and extracted with EA (5 mL*3). The organic layers were combined, dried over Na₂SO₄ and concentrated to give (S)-2-((4-((2-(4-chlorobenzyl)pyrimidin-4-yl)oxy)piperidin-1-yl)methyl)-1-(oxetan-2-ylmethyl)-1H-benzo[d]imidazole-5-carbonitrile (130 mg, yield: 52%) as a yellow oil.

MS Calcd.: 528.2; MS Found: 529.2[M+H]⁺.

### Step F: (S)-2-((4-((2-(4-chlorobenzyl)pyrimidin-4-yl)oxy)piperidin-1-yl)methyl)-N-hydroxy-1-(oxetan-2-ylmethyl)-1H-benzo[d]imidazole-5-carbimidic acid

To a solution of (S)-2-((4-((2-(4-chlorobenzyl)pyrimidin-4-yl)oxy)piperidin-1-yl)methyl)-1-(oxetan-2-ylmethyl)-1H-benzo[d]imidazole-5-carbonitrile (250 mg, 0.47 mmol) in EtOH (4 mL) was added NH₂OH·HCl (197 mg, 2.84 mmol) and TEA (475 mg, 4.7 mmol) successively. The reaction was stirred at 90° C. for 2 h. After the reaction was completed, the reaction was quenched with H₂O and extracted with EA (5 mL*3). The organic layers were combined, dried over Na₂SO₄ and concentrated. the reaction was purified by HPLC to give (S,E)-2-((4-((2-(4-chlorobenzyl)pyrimidin-4-yl)oxy)piperidin-1-yl)methyl)-N-hydroxy-1-(oxetan-2-ylmethyl)-1H-benzo[d]imidazole-5-carbimidic acid (130 mg, yield: 52%) as a colourless oil.

MS Calcd.: 562.2; MS Found: 281.7[M/2+H]+.

### Step G: (S)-3-(2-((4-((2-(4-chlorobenzyl)pyrimidin-4-yl)oxy)piperidin-1-yl)methyl)-1-(oxetan-2-ylmethyl)-1H-benzo[d]imidazol-5-yl)-5-(trifluoromethyl)-1,2,4-oxadiazole

To a solution of (S)-2-((4-((2-(4-chlorobenzyl)pyrimidin-4-yl)oxy)piperidin-1-yl)methyl)-N-hydroxy-1-(oxetan-2-ylmethyl)-1H-benzo[d]imidazole-5-carbimidic acid (130 mg, 0.23 mmol) in THE (3 mL) was added TFAA (194 mg, 0.93 mmol). The reaction was stirred at 30° C. for 2 h. After the reaction was completed, the reaction was quenched with H₂O and extracted with EA (5 mL*3). The organic layers were combined, dried over Na₂SO₄ and concentrated. to give (S)-3-(2-((4-((2-(4-chlorobenzyl)pyrimidin-4-yl)oxy)piperidin-1-yl)methyl)-1-(oxetan-2-ylmethyl)-1H-benzo[d]imidazol-5-yl)-5-(trifluoromethyl)-1,2,4-oxadiazole (180 mg, yield: 138%) as colorless oil.

MS Calcd.: 639.2; MS Found: 640.8[M+H]⁺

### Step H: 2-{[4-({2-[(4-chlorophenyl)methyl]pyrimidin-4-yl}oxy)piperidin-1-yl]methyl}-1-{[(2S)-oxetan-2-yl]methyl}-5-[5-(trifluoromethyl)-4H-1,2,4-triazol-3-yl]-1H-1,3-benzodiazole (Compound 106a)

To a solution of (S)-3-(2-((4-((2-(4-chlorobenzyl)pyrimidin-4-yl)oxy)piperidin-1-yl)methyl)-1-(oxetan-2-ylmethyl)-1H-benzo[d]imidazol-5-yl)-5-(trifluoromethyl)-1,2,4-oxadiazole (180 mg, 0.28 mmol) in DMF (2 mL) was added N₂H₄·H₂O (42.3 mg, 0.85 mmol). The reaction was stirred at rt for 2 h. After the reaction was completed, the reaction was purified by Prep-HPLC to give Compound 106a (25.47 mg, yield: 14%) as a white solid.

MS Calcd.: 638.2; MS Found: 639.5 [M+H]⁺.

¹H NMR (400 MHz, DMSO-d₆): δ 8.42 (d, J=5.2 Hz, 1H), 8.29 (br s, 1H), 7.82-7.96 (m, 2H), 7.31-7.36 (m, 4H), 6.74 (d, J=5.6 Hz, 1H), 5.01-5.13 (m, 2H), 4.74-4.81 (m, 1H), 4.60-4.67 (m, 1H), 4.48-4.53 (m, 1H), 4.37-4.42 (m, 1H), 4.09 (s, 2H), 3.90-4.01 (m, 1H), 3.79-3.90 (m, 1H), 2.67-2.83 (m, 3H), 2.32-2.50 (m, 3H), 1.87-2.10 (m, 2H), 1.55-1.75 (s, 2H).

### Example 7: 2-{[4-({2-[(4-chlorophenyl)methyl]pyrimidin-4-yl}oxy)piperidin-1-yl]methyl}-1-{[(2S)-oxetan-2-yl]methyl}-5-(1H-1,2,3,4-tetrazol-5-yl)-1H-1,3-benzodiazole (Compound 107a)

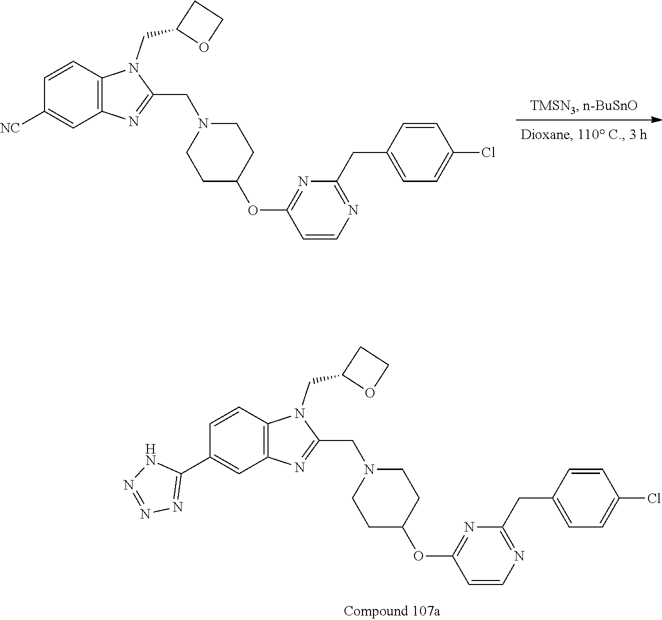 [SMILES: N#Cc1ccc2c(c1)nc(CN1CCC(Oc3ccnc(Cc4ccc(Cl)cc4)n3)CC1)n2C[C@@H]1CCO1 ; Clc1ccc(Cc2nccc(OC3CCN(Cc4nc5cc(-c6nnn[nH]6)ccc5n4C[C@@H]4CCO4)CC3)n2)cc1]

To a solution of (S)-2-((4-((2-(4-chlorobenzyl)pyrimidin-4-yl)oxy)piperidin-1-yl)methyl)-5-isocyano-1-(oxetan-2-ylmethyl)-1H-benzo[d]imidazole (150 mg, 0.28 mmol) in Dioxane (2 mL) was added TMSN₃ (153.9 mg, 1.35 mmol) and n-BuSnO (133.5 mg, 0.54 mmol). The resulting mixture was stirred at 110° C. for 3 hours. The solvent was removed in vacuo. The residue was purified by Prep-HPLC to give Compound 107a (23.15 mg, yield: 14%) as a white solid.

MS Calcd.: 571.2; MS Found: 572.3 [M+H]⁺.

¹H NMR (400 MHz, DMSO-d₆): 8.41 (d, J=5.6 Hz, 1H), 8.22 (d, J=0.8 Hz, 1H), 7.92 (dd, J=8.8 Hz, 0.8 Hz, 1H), 7.78 (d, J=8.4 Hz, 1H), 7.31-7.36 (m, 4H), 6.73 (d, J=5.6 Hz, 1H), 4.98-5.14 (m, 2H), 4.72-4.78 (m, 1H), 4.58-4.63 (m, 1H), 4.47-4.53 (m, 1H), 4.36-4.42 (m, 1H), 4.08 (s, 2H), 3.94 (d, J=13.6 Hz, 1H), 3.81 (d, J=13.6 Hz, 1H), 2.67-2.82 (m, 3H), 2.32-2.46 (m, 3H), 1.88-1.97 (m, 2H), 1.58-1.67 (m, 2H).

### Example 8: 3-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)-4,5-dihydro-1,2,4-oxadiazol-5-one (Compound 108a)

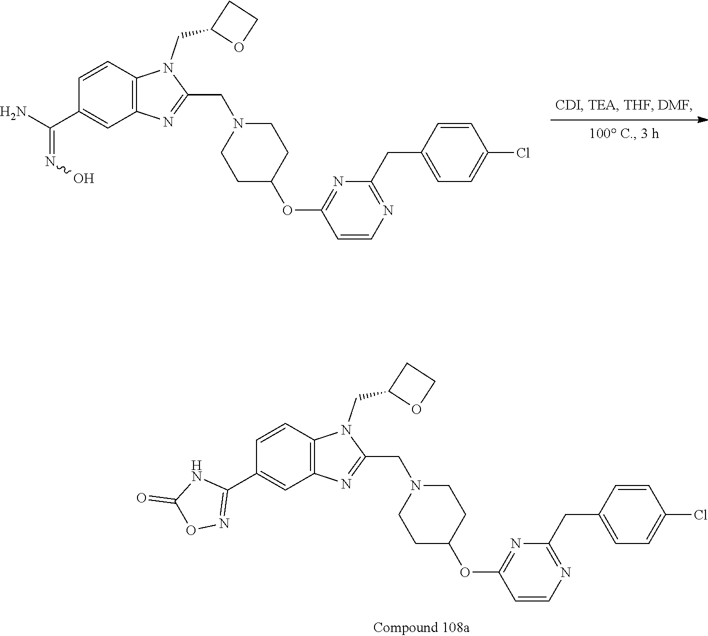 [SMILES: NC(=NO)c1ccc2c(c1)nc(CN1CCC(Oc3ccnc(Cc4ccc(Cl)cc4)n3)CC1)n2C[C@@H]1CCO1 ; O=c1[nH]c(-c2ccc3c(c2)nc(CN2CCC(Oc4ccnc(Cc5ccc(Cl)cc5)n4)CC2)n3C[C@@H]2CCO2)no1]

To a solution of (S)-2-((4-((2-(4-chlorobenzyl)pyrimidin-4-yl)oxy)piperidin-1-yl)methyl)-N′-hydroxy-1-(oxetan-2-ylmethyl)-1H-benzo[d]imidazole-5-carboximidamide (170 mg, 0.3 mmol) in THF/DMF (4 mL/4 mL) was added TEA (90 mg, 0.9 mmol) and CDI (102.86 mg, 1.86 mmol). The resulting mixture was stirred at 100° C. for 3 hours. The solvent was removed in vacuo. The residue was purified by Prep-HPLC to give Compound 108a (16.66 mg, yield: 9.36%) as a white solid.

MS Calcd.: 587.2; MS Found: 588.3 [M+H]⁺.

¹H NMR (400 MHz, DMSO-d₆): 8.41 (d, J=6.0 Hz, 1H), 8.04 (d, J=0.8 Hz, 1H), 7.80 (d, J=8.4 Hz, 1H), 7.69 (dd, J=8.4 Hz, 1.2 Hz, 1H), 7.30-7.38 (m, 4H), 6.73 (d, J=6.0 Hz, 1H), 4.98-5.13 (m, 2H), 4.72-4.79 (m, 1H), 4.58-4.64 (m, 1H), 4.46-4.52 (m, 1H), 4.35-4.41 (m, 1H), 4.08 (s, 2H), 3.93 (d, J=13.6 Hz, 1H), 3.80 (d, J=13.6 Hz, 1H), 2.66-2.80 (m, 3H), 2.32-2.46 (m, 3H), 1.88-1.98 (m, 2H), 1.55-1.69 (m, 2H).

### Example 9: 3-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)-4,5-dihydro-1,2,4-oxadiazol-5-one (Compound 109a)

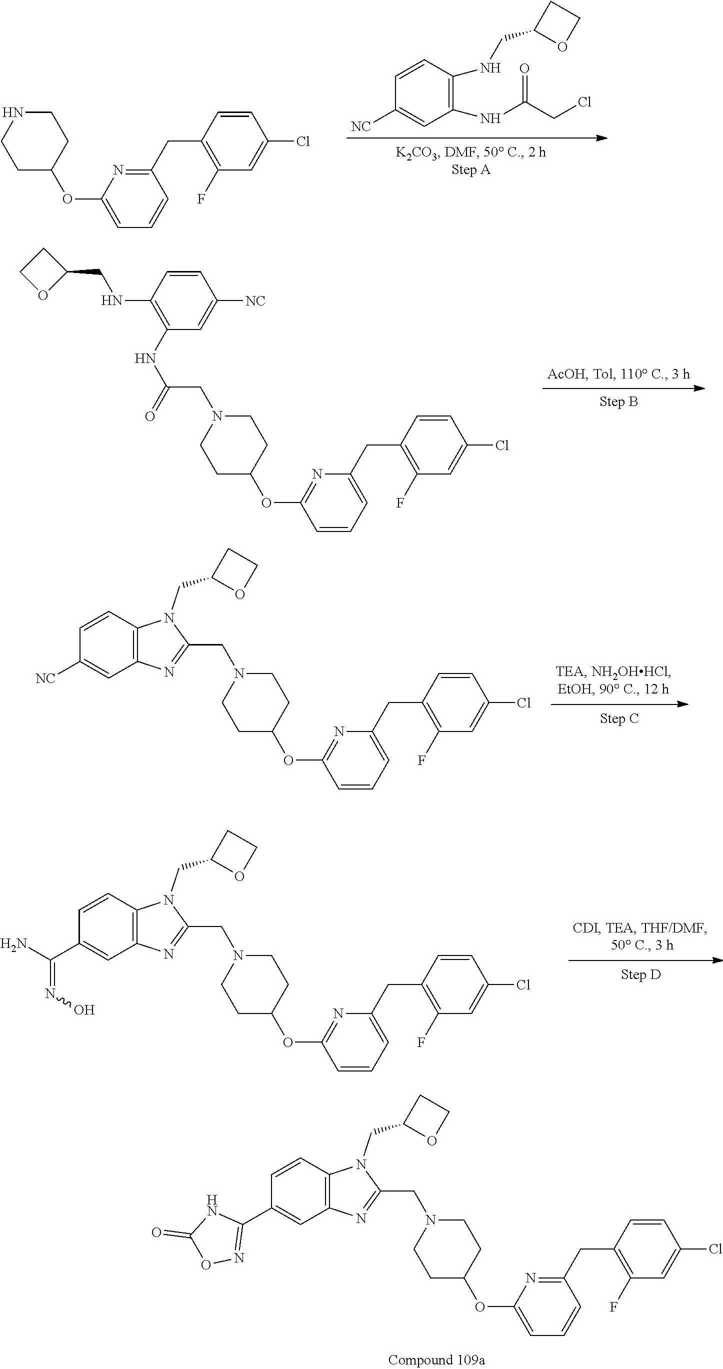

### Step A: (S)-2-(4-((6-(4-chloro-2-fluorobenzyl)pyridin-2-yl)oxy)piperidin-1-yl)-N-(5-isocyano-2-((oxetan-2-ylmethyl)amino)phenyl)acetamide

To a solution of 2-(4-chloro-2-fluorobenzyl)-6-(piperidin-4-yloxy)pyridine (640 mg, 2.0 mmol) in DMF (5 mL) was added K₂CO₃ (828 mg, 6.0 mmol) and (S)-2-chloro-N-(5-cyano-2-((oxetan-2-ylmethyl)amino)phenyl)acetamide (558 mg, 2.0 mmol). The resulting mixture was stirred at 50° C. for 2 hours. The reaction was filtered and concentrated to give (S)-2-(4-((6-(4-chloro-2-fluorobenzyl)pyridin-2-yl)oxy)piperidin-1-yl)-N-(5-isocyano-2-((oxetan-2-ylmethyl)amino)phenyl)acetamide (470 mg, crude) as brown oil.

MS Calcd.: 563.2; MS Found: 564.2 [M+H]⁺.

### Step B: (S)-2-((4-((6-(4-chloro-2-fluorobenzyl)pyridin-2-yl)oxy)piperidin-1-yl)methyl)-5-isocyano-1-(oxetan-2-ylmethyl)-1H-benzo[d]imidazole

To a solution of (S)-2-(4-((6-(4-chloro-2-fluorobenzyl)pyridin-2-yl)oxy)piperidin-1-yl)-N-(5-isocyano-2-((oxetan-2-ylmethyl)amino)phenyl) acetamide (540 mg, 0.96 mmol) in Toluene (6 mL) was added AcOH (1 mL). The resulting mixture was stirred at 110° C. for 3 hours. After the reaction was completed, the reaction mixture was filtered and the filtrate concentrated. The crude product was purified by column chromatography on silica gel (DCM:MeOH=70:1) to give (S)-2-((4-((6-(4-chloro-2-fluorobenzyl)pyridin-2-yl)oxy)piperidin-1-yl)methyl)-5-isocyano-1-(oxetan-2-ylmethyl)-1H-benzo[d]imidazole (330 mg, yield: 64%) as a purple solid.

MS Calcd.: 545.2; MS Found: 546.2 [M+H]⁺.

### Step C: (S)-2-((4-((6-(4-chloro-2-fluorobenzyl)pyridin-2-yl)oxy)piperidin-1-yl)methyl)-N′-hydroxy-1-(oxetan-2-ylmethyl)-1H-benzo[d]imidazole-5-carboximidamide

To a solution of (S)-2-((4-((6-(4-chloro-2-fluorobenzyl)pyridin-2-yl)oxy)piperidin-1-yl)methyl)-5-isocyano-1-(oxetan-2-ylmethyl)-1H-benzo[d]imidazole (195 mg, 0.36 mmol) in EtOH (7 mL) was added TEA (144 mg, 4.12 mmol) and NH₂OH·HCl (149 mg, 2.16 mmol). The resulting mixture was stirred at 90° C. for 12 hours. After the reaction was completed, the reaction mixture was filtered and the filtrate concentrated. The crude product was purified by column chromatography on silica gel (DCM:MeOH=20:1) to give (S)-2-((4-((6-(4-chloro-2-fluorobenzyl)pyridin-2-yl)oxy)piperidin-1-yl)methyl)-N′-hydroxy-1-(oxetan-2-ylmethyl)-1H-benzo[d]imidazole-5-carboximidamide (370 mg, crude) as a yellow solid.

MS Calcd.: 578.2; MS Found: 579.3 [M+H]⁺.

### Step D: 3-(2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazol-5-yl)-4,5-dihydro-1,2,4-oxadiazol-5-one (Compound 109a)

To a solution of (S)-2-((4-((6-(4-chloro-2-fluorobenzyl)pyridin-2-yl)oxy)piperidin-1-yl)methyl)-N′-hydroxy-1-(oxetan-2-ylmethyl)-1H-benzo[d]imidazole-5-carboximidamide (220 mg, 0.38 mmol) in THF/DMF (1.5 mL/1.5 mL) was added TEA (114 mg, 1.14 mmol) and CDI (92 mg, 0.57 mmol). The resulting mixture was stirred at 50° C. for 3 hours. The solvent was removed in vacuo. The residue was purified by Prep-HPLC to give Compound 109a (26.48 mg, yield: 12%) as a white solid.

MS Calcd.: 604.2; MS Found: 605.3 [M+H]⁺.

¹H NMR (400 MHz, DMSO-d₆): 8.04 (d, J=1.6 Hz, 1H), 7.80 (d, J=8.8 Hz, 1H), 7.69 (dd, J=8.4 Hz, 1.6 Hz, 1H), 7.58-7.65 (m, 1H), 7.34-7.39 (m, 2H), 7.23 (dd, J=8.4 Hz, 2.0 Hz, 1H), 6.82 (d, J=7.2 Hz, 1H), 6.58 (d, J=8.4 Hz, 1H), 5.06-5.14 (m, 1H), 4.72-4.84 (m, 2H), 4.57-4.63 (m, 1H), 4.46-4.52 (m, 1H), 4.35-4.41 (m, 1H), 4.01 (s, 2H), 3.92 (d, J=13.2 Hz, 1H), 3.78 (d, J=13.6 Hz, 1H), 2.65-2.80 (m, 3H), 2.37-2.46 (m, 1H), 2.20-2.35 (m, 2H), 1.80-1.89 (m, 2H), 1.47-1.58 (m, 2H).

### Example 10: 2-{[4-({6-[(4-chloro-2-fluorophenyl)methyl]pyridin-2-yl}oxy)piperidin-1-yl]methyl}-1-{[(2S)-oxetan-2-yl]methyl}-5-(1H-1,2,3,4-tetrazol-5-yl)-1H-1,3-benzodiazole (Compound 110a)

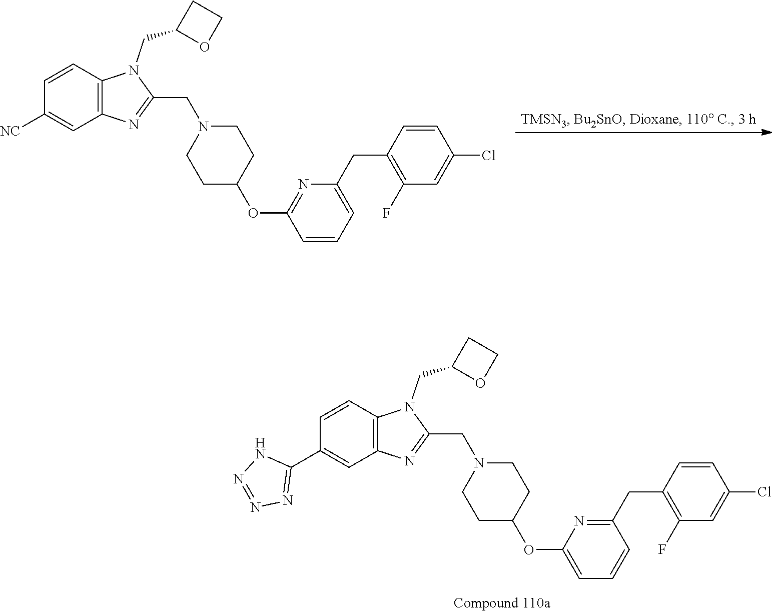 [SMILES: N#Cc1ccc2c(c1)nc(CN1CCC(Oc3cccc(Cc4ccc(Cl)cc4F)n3)CC1)n2C[C@@H]1CCO1 ; Fc1cc(Cl)ccc1Cc1cccc(OC2CCN(Cc3nc4cc(-c5nnn[nH]5)ccc4n3C[C@@H]3CCO3)CC2)n1]

To a solution of (S)-2-((4-((6-(4-chloro-2-fluorobenzyl)pyridin-2-yl)oxy)piperidin-1-yl)methyl)-5-isocyano-1-(oxetan-2-ylmethyl)-1H-benzo[d]imidazole (200 mg, 0.37 mmol) in Dioxane (3 mL) was added TMSN₃ (211 mg, 1.84 mmol) and n-Bu₂SnO (184 mg, 0.74 mmol). The resulting mixture was stirred at 110° C. for 3 hours. The solvent was removed in vacuo. The residue was purified by Prep-HPLC to give Compound 110a (23.15 mg, yield: 14%) as a white solid.

MS Calcd.: 588.2; MS Found: 589.2 [M+H]⁺.

¹H NMR (400 MHz, DMSO-d₆): 8.27 (s, 1H), 7.94 (dd, J=8.4 Hz, 1.6 Hz, 1H), 7.85 (d, J=8.4 Hz, 1H), 7.60 (t, J=7.6 Hz, 1H), 7.35-7.41 (m, 2H), 7.24 (dd, J=8.4 Hz, 2.0 Hz, 1H), 6.82 (d, J=7.2 Hz, 1H), 6.58 (d, J=8.4 Hz, 1H), 5.05-5.14 (m, 1H), 4.72-4.87 (m, 2H), 4.60-4.67 (m, 1H), 4.48-4.53 (m, 1H), 4.37-4.42 (m, 1H), 3.95-4.03 (m, 3H), 3.86 (d, J=13.6 Hz, 1H), 2.67-2.87 (m, 3H), 2.30-2.49 (m, 3H), 1.84-1.94 (m, 2H), 1.51-1.63 (m, 2H).

### Example 11: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)pyridine-3-carboxamide (Compound 111a)

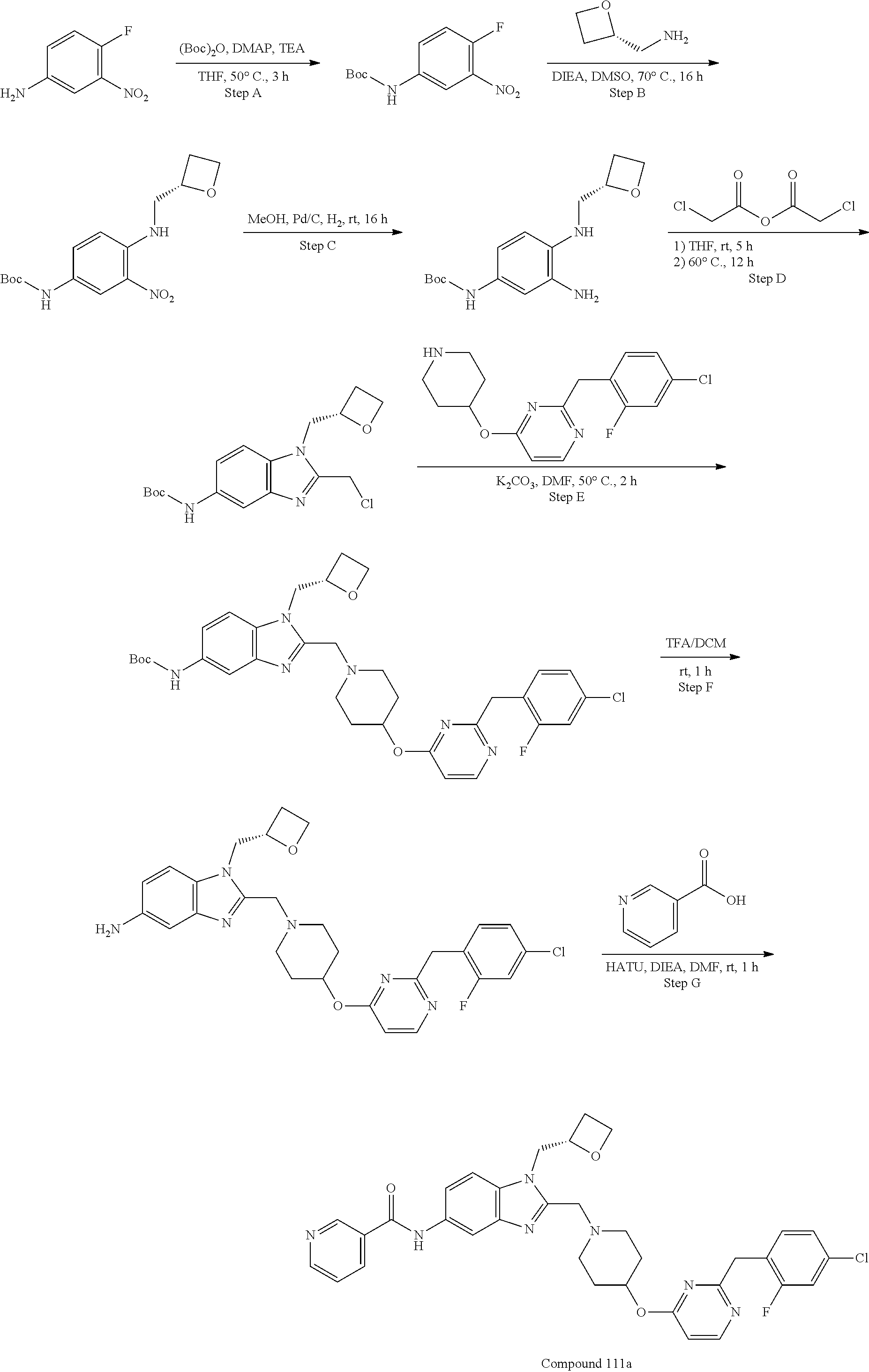

### Step A: tert-butyl (4-fluoro-3-nitrophenyl)carbamate

A mixture of 4-fluoro-3-nitroaniline (30 g, 192 mmol), (Boc)₂O (50.3 g, 231 mmol), DMAP (2.3 g, 18.85 mmol) and TEA (79.4 mL, 570 mmol) in THF (500 mL) was stirred at 50° C. for 3 hours. After the reaction was completed, the mixture was filtered and concentrated in vacuum to give the crude product. The residue was purified by column chromatography (PE:EA=5:1) to give tert-butyl (4-fluoro-3-nitrophenyl)carbamate (41.2 mg, yield: 83%) as a yellow solid.

MS Calcd.: 256.09; MS Found: 255.1 [M−H]⁻.

### Step B: tert-butyl (S)-(3-nitro-4-((oxetan-2-ylmethyl)amino)phenyl)carbamate

A mixture of (S)-oxetan-2-ylmethanamine (12 g, 65.4 mmol), DIEA (21.17 g, 164.07 mmol) and tert-butyl (4-fluoro-3-nitrophenyl)carbamate (14 g, 54.69 mmol) in DMSO (200 mL) was stirred at 70° C. for 16 hours. After the reaction was completed, the reaction was quenched with 500 mL H₂O. Then the mixture was extracted with ethyl acetate (600 mL×3). The organic layer was combined and dried with Na₂SO₄. Then the mixture was filtered and concentrated in vacuum to give the crude product. The residue was purified by column chromatography (PE:EA=3:1) to give tert-butyl (S)-(3-nitro-4-((oxetan-2-ylmethyl)amino)phenyl)carbamate (11.8 g, yield: 67%) as a red solid.

### Step C: tert-butyl (S)-(3-amino-4-((oxetan-2-ylmethyl)amino)phenyl)carbamate

A mixture of tert-butyl (S)-(3-nitro-4-((oxetan-2-ylmethyl)amino)phenyl)carbamate (11.8 mg, 36.53 mmol) and Pd/C (10% w/w, 1.1 g) in MeOH (120 mL) was stirred at room temperature for 16 hours under H₂. After the reaction was completed, the mixture was filtered and the filtrate concentrated in vacuum to give tert-butyl (S)-(3-amino-4-((oxetan-2-ylmethyl)amino)phenyl)carbamate (14.3 mg, crude) as a purple solid.

MS Calcd.: 293.2; MS Found: 294.2 [M+H]⁺.

### Step D: tert-butyl (S)-(2-(chloromethyl)-1-(oxetan-2-ylmethyl)-1H-benzo[d]imidazol-5-yl)carbamate

A mixture of tert-butyl (S)-(3-amino-4-((oxetan-2-ylmethyl)amino)phenyl)carbamate (13.3 g) and 2-chloroacetic anhydride (7.76 g, 45.37 mmol) in THE (130 mL) was stirred at room temperature for 5 hours. Then the reaction was stirred at 60° C. for 12 hours. After the reaction was completed, the reaction was concentrated and the residue was purified by column chromatography (PE:EA=1:2) to give tert-butyl (S)-(2-(chloromethyl)-1-(oxetan-2-ylmethyl)-1H-benzo[d]imidazol-5-yl)carbamate (14.2 mg, yield: 83%) as purple oil.

MS Calcd.: 351.1; MS Found: 352.1 [M+H]⁺.

### Step E: tert-butyl (S)-(2-((4-((2-(4-chloro-2-fluorobenzyl)pyrimidin-4-yl)oxy)piperidin-1-yl)methyl)-1-(oxetan-2-ylmethyl)-1H-benzo[d]imidazol-5-yl)carbamate

To a solution of tert-butyl (S)-(2-(chloromethyl)-1-(oxetan-2-ylmethyl)-1H-benzo[d]imidazol-5-yl)carbamate (518 mg, 1.47 mmol) in DMF (10 mL) was added K₂CO₃ (608 mg, 4.41 mmol) and 2-(4-chloro-2-fluorobenzyl)-4-(piperidin-4-yloxy)pyrimidine (640 mg). The resulting mixture was stirred at 50° C. for 2 hours. The reaction was filtered and concentrated under reduced pressure. The residue was purified by column chromatography on silica gel to give tert-butyl (S)-(2-((4-((2-(4-chloro-2-fluorobenzyl)pyrimidin-4-yl)oxy)piperidin-1-yl)methyl)-1-(oxetan-2-ylmethyl)-1H-benzo[d]imidazol-5-yl)carbamate (790 mg, yield: 84%) as a yellow solid.

MS Calcd.: 636.3; MS Found: 637.1 [M+H]⁺.

### Step F: (S)-2-((4-((2-(4-chloro-2-fluorobenzyl)pyrimidin-4-yl)oxy)piperidin-1-yl)methyl)-1-(oxetan-2-ylmethyl)-1H-benzo[d]imidazol-5-amine

To a solution of tert-butyl (S)-(2-((4-((2-(4-chloro-2-fluorobenzyl)pyrimidin-4-yl)oxy)piperidin-1-yl)methyl)-1-(oxetan-2-ylmethyl)-1H-benzo[d]imidazol-5-yl)carbamate (790 mg, 1.24 mmol) in DCM (5 mL) was added TFA (1 mL). The resulting mixture was stirred at room temperature for 1 hours. The solvent was removed under vacuum to obtain (S)-2-((4-((2-(4-chloro-2-fluorobenzyl)pyrimidin-4-yl)oxy)piperidin-1-yl)methyl)-1-(oxetan-2-ylmethyl)-1H-benzo[d]imidazol-5-amine (50 mg, crude) as a yellow solid.

MS Calcd.: 536.2; MS Found: 537.2 [M+H]⁺.

### Step G: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)pyridine-3-carboxamide (Compound 111a)

To a solution of (S)-2-((4-((2-(4-chloro-2-fluorobenzyl)pyrimidin-4-yl)oxy)piperidin-1-yl)methyl)-1-(oxetan-2-ylmethyl)-1H-benzo[d]imidazol-5-amine (80 mg, 0.15 mmol) in DMF (2 mL) was added nicotinic acid (27 mg, 0.22 mmol), DIEA (58 mg, 0.45 mmol) and HATU (74 mg, 0.19 mmol). The resulting mixture was stirred at room temperature for 1 hour, LC-MS showed the reaction was completed. The residue was purified by Prep-HPLC to give Compound 111a (10.99 mg, yield: 12%) as a white solid.

MS Calcd.: 641.2; MS Found: 642.5 [M+H]⁺.

¹H NMR (400 MHz, DMSO-d₆): δ 10.41 (s, 1H), 9.14 (s, 1H), 8.76 (d, J=4.0 Hz, 1H), 8.40 (d, J=6.0 Hz, 1H), 8.32 (d, J=8.0 Hz, 1H), 8.05 (s, 1H), 7.50-7.61 (m, 3H), 7.35-7.42 (m, 2H), 7.24-7.28 (m, 1H), 6.73 (d, J=6.0 Hz, 1H), 5.06-5.12 (m, 1H), 4.84-4.92 (m, 1H), 4.64-4.71 (m, 1H), 4.47-4.59 (m, 2H), 4.32-4.41 (m, 1H), 4.15 (s, 2H), 3.88 (d, J=13.2 Hz, 1H), 3.75 (d, J=13.6 Hz, 1H), 2.65-2.80 (m, 3H), 2.38-2.50 (m, 1H), 2.20-2.29 (m, 2H), 1.85-1.93 (m, 2H), 1.54-1.64 (m, 2H).

### Example 12: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)-6-methoxypyridine-3-carboxamide (Compound 112a)

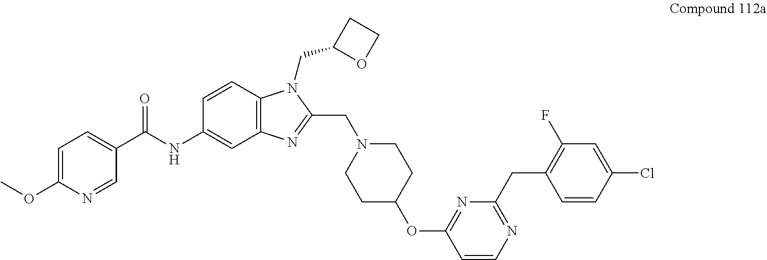 [SMILES: COc1ccc(C(=O)Nc2ccc3c(c2)nc(CN2CCC(Oc4ccnc(Cc5ccc(Cl)cc5F)n4)CC2)n3C[C@@H]2CCO2)cn1]

The synthesis of Compound 112a was similar to that of Compound 111a except that 6-methoxypyridine-3-carboxylic acid was used instead of pyridine-3-carboxylic acid. Compound 112a (39.39 mg, yield: 52%) was obtained as a white solid.

MS Calcd.: 671.2; MS Found: 672.3 [M+H]⁺.

¹H NMR (400 MHz, DMSO-d₆) δ 10.21 (s, 1H), 8.81 (d, J=2.4 Hz, 1H), 8.40 (d, J=6.0 Hz, 1H), 8.26 (dd, J=8.8 Hz, 2.4 Hz, 1H), 8.02 (d, J=2.0 Hz, 1H), 7.51-7.59 (m, 2H), 7.37-7.42 (m, 2H), 7.25 (dd, J=8.0 Hz, 2.0 Hz, 1H), 6.96 (d, J=8.8 Hz, 1H), 6.73 (d, J=6.0 Hz, 1H), 5.05-5.12 (m, 1H), 4.85-4.93 (m, 1H), 4.63-4.71 (m, 1H), 4.47-4.57 (m, 2H), 4.34-4.40 (m, 1H), 4.15 (s, 2H), 3.94 (s, 3H), 3.87 (d, J=13.6 Hz, 1H), 3.74 (d, J=13.6 Hz, 1H), 2.65-2.79 (m, 3H), 2.37-2.48 (m, 1H), 2.20-2.29 (m, 2H), 1.84-1.94 (m, 2H), 1.54-1.63 (m, 2H).

### Example 13: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)-6-methylpyridine-3-carboxamide (Compound 113a)

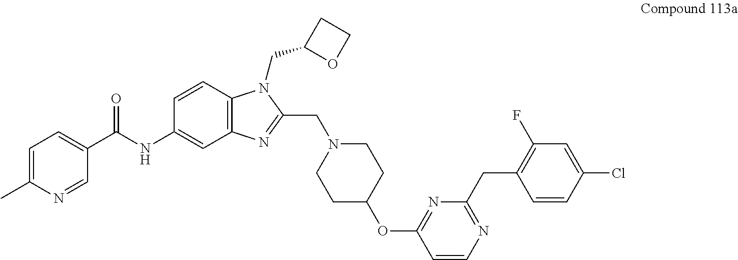 [SMILES: Cc1ccc(C(=O)Nc2ccc3c(c2)nc(CN2CCC(Oc4ccnc(Cc5ccc(Cl)cc5F)n4)CC2)n3C[C@@H]2CCO2)cn1]

The synthesis of Compound 113a was similar to that of Compound 111a except that 6-methylpyridine-3-carboxylic acid was used instead of pyridine-3-carboxylic acid. Compound 113a (41.13 mg, yield: 67%) was obtained as a white solid.

MS Calcd.: 655.3; MS Found: 656.3 [M+H]⁺.

¹H NMR (400 MHz, DMSO-d₆): 10.32 (s, 1H), 9.03 (d, J=2.0 Hz, 1H), 8.41 (d, J=5.6 Hz, 1H), 8.23 (dd, J=8.4 Hz, 2.4 Hz, 1H), 8.05 (s, 1H), 7.52-7.61 (m, 2H), 7.38-7.44 (m, 3H), 7.26 (dd, J=8.4 Hz, 2.0 Hz, 1H), 6.74 (d, J=5.6 Hz, 1H), 5.07-5.13 (m, 1H), 4.86-4.94 (m, 1H), 4.66-4.72 (m, 1H), 4.48-4.58 (m, 2H), 4.36-4.41 (m, 1H), 4.16 (s, 2H), 3.89 (d, J=13.2 Hz, 1H), 3.75 (d, J=13.6 Hz, 1H), 2.68-2.80 (m, 3H), 2.57 (s, 3H), 2.38-2.48 (m, 1H), 2.20-2.35 (m, 2H), 1.85-1.93 (m, 2H), 1.53-1.68 (m, 2H).

### Example 14: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)-6-ethylpyridine-3-carboxamide (Compound 114a)

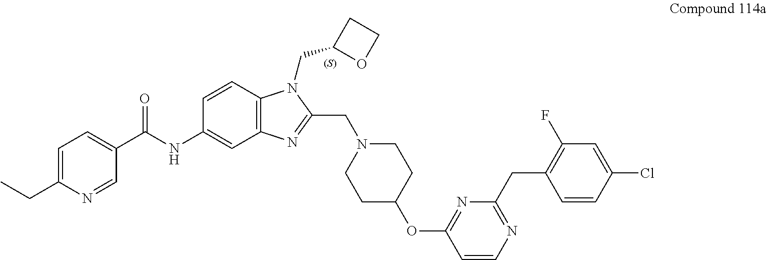

The synthesis of Compound 114a was similar to that of Compound 111a except that 6-ethylpyridine-3-carboxylic acid was used instead of pyridine-3-carboxylic acid. Compound 114a (21.7 mg, yield: 34.8%) was obtained as a white solid.

MS Calcd.: 669.3; MS Found: 670.4 [M+H]⁺.

¹H NMR (400 MHz, DMSO-d₆): δ 10.32 (s, 1H), 9.04 (d, J=2.0 Hz, 1H), 8.40 (d, J=5.6 Hz, 1H), 8.23 (dd, J=8.4 Hz, 2.4 Hz, 1H), 8.04 (d, J=1.6 Hz, 1H), 7.51-7.61 (m, 2H), 7.37-7.44 (m, 3H), 7.25 (dd, J=8.4 Hz, 2.0 Hz, 1H), 6.73 (d, J=5.6 Hz, 1H), 5.06-5.12 (m, 1H), 4.85-4.91 (m, 1H), 4.65-4.71 (m, 1H), 4.47-4.58 (m, 2H), 4.34-4.40 (m, 1H), 4.15 (s, 2H), 3.87 (d, J=13.2 Hz, 1H), 3.75 (d, J=13.6 Hz, 1H), 2.84 (q, J=7.6 Hz, 2H), 2.64-2.78 (m, 3H), 2.39-2.46 (m, 1H), 2.20-2.31 (m, 2H), 1.85-1.92 (m, 2H), 1.52-1.64 (m, 2H), 1.27 (t, J=7.6 Hz, 3H).

### Example 15: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)-6-(2-methoxyethoxy)pyridine-3-carboxamide (Compound 115a)

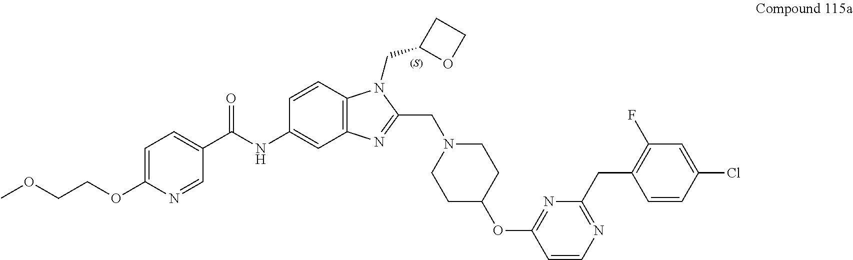

The synthesis of Compound 115a was similar to that of Compound 111a except that 6-(2-methoxyethoxy)nicotinic acid was used instead of pyridine-3-carboxylic acid. Compound 115a (22.5 mg, yield: 28.1%) was finally obtained as a white solid.

MS Calcd.: 715.3; MS Found: 716.5 [M+H]⁺.

¹H NMR (400 MHz, DMSO-d₆) δ10.21 (s, 1H), 8.79 (d, J=2.4 Hz, 1H), 8.41 (d, J=6.0 Hz, 1H), 8.26 (dd, J=8.8 Hz, 2.8 Hz, 1H), 8.02 (d, J=1.6 Hz, 1H), 7.51-7.60 (m, 2H), 7.37-7.42 (m, 2H), 7.25 (dd, J=8.0 Hz, 2.0 Hz, 1H), 6.96 (d, J=8.8 Hz, 1H), 6.73 (d, J=5.6 Hz, 1H), 5.05-5.11 (m, 1H), 4.85-4.91 (m, 1H), 4.65-4.70 (m, 1H), 4.46-4.57 (m, 4H), 4.34-4.40 (m, 1H), 4.15 (s, 2H), 3.88 (d, J=13.6 Hz, 1H), 3.75 (d, J=13.2 Hz, 1H), 3.68-3.70 (m, 2H), 3.31 (s, 3H), 2.64-2.78 (m, 3H), 2.39-2.49 (m, 1H), 2.21-2.31 (m, 2H), 1.85-1.92 (m, 2H), 1.52-1.63 (m, 2H).

### Example 16: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)-6-(propan-2-yl)pyridine-3-carboxamide (Compound 116a)

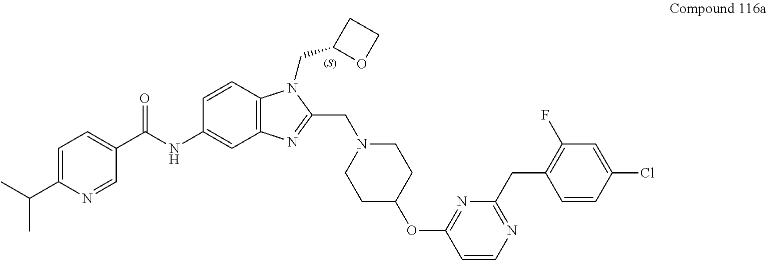

The synthesis of Compound 116a was similar to that of Compound 111a except that 6-isopropylnicotinic acid was used instead of pyridine-3-carboxylic acid. Compound 116a (15.69 mg, yield: 24%) was finally obtained as a white solid.

MS Calcd.: 683.3; MS Found: 684.4 [M+H]⁺.

¹H NMR (400 MHz, DMSO-d₆): 10.32 (s, 1H), 9.04 (d, J=2.0 Hz, 1H), 8.41 (d, J=6.0 Hz, 1H), 8.23 (dd, J=8.4 Hz, 2.4 Hz, 1H), 8.04 (d, J=1.6 Hz, 1H), 7.53-7.60 (m, 2H), 7.37-7.46 m, 3H), 7.25 (dd, J=8.4 Hz, 2.0 Hz, 1H), 6.73 (d, J=5.6 Hz, 1H), 5.05-5.133 (m, 1H), 4.85-4.93 (m, 1H), 4.63-4.72 (m, 1H), 4.45-4.59 (m, 2H), 4.34-4.40 (m, 1H), 4.15 (s, 2H), 3.87 (d, J=13.6 Hz, 1H), 3.75 (d, J=13.6 Hz, 1H), 3.08-3.16 (m, 1H), 2.64-2.78 (m, 3H), 2.39-2.49 (m, 1H), 2.21-2.30 (m, 2H), 1.87-1.91 (m, 2H), 1.52-1.63 (m, 2H), 1.28 (d, J=6.8 Hz, 6H).

### Example 17: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)-6-cyclopropylpyridine-3-carboxamide (Compound 117a)

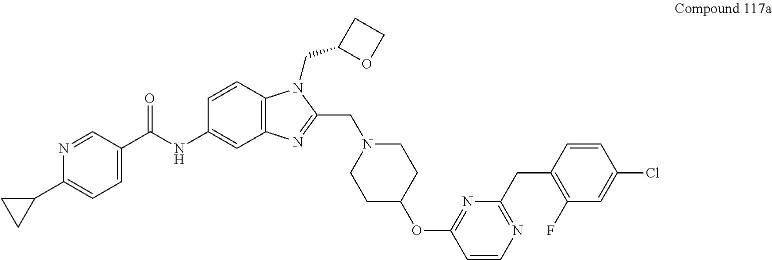 [SMILES: O=C(Nc1ccc2c(c1)nc(CN1CCC(Oc3ccnc(Cc4ccc(Cl)cc4F)n3)CC1)n2C[C@@H]1CCO1)c1ccc(C2CC2)nc1]

The synthesis of Compound 117a was similar to that of Compound 111a except that 6-cyclopropylnicotinic acid was used instead of pyridine-3-carboxylic acid. Compound 117a (39.34 mg, yield: 51%) was finally obtained as a white solid.

MS Calcd.: 681.3; MS Found: 682.4 [M+H]⁺.

¹H NMR (400 MHz, DMSO-d₆): 10.26 (s, 1H), 8.95 (d, J=1.6 Hz, 1H), 8.40 (d, J=6.0 Hz, 1H), 8.17 (dd, J=8.4 Hz, 2.4 Hz, 1H), 8.03 (s, 1H), 7.50-7.59 (m, 2H), 7.37-7.48 (m, 3H), 7.25 (dd, J=8.0 Hz, 1.6 Hz, 1H), 6.73 (d, J=6.0 Hz, 1H), 5.05-5.11 (m, 1H), 4.83-4.93 (m, 1H), 4.62-4.71 (m, 1H), 4.45-4.59 (m, 2H), 4.32-4.39 (m, 1H), 4.15 (s, 2H), 3.88 (d, J=13.6 Hz, 1H), 3.74 (d, J=14.0 Hz, 1H), 2.66-2.81 (m, 3H), 2.35-2.49 (m, 1H), 2.18-2.32 (m, 3H), 1.84-1.92 (m, 2H), 1.52-1.64 (m, 2H), 1.00-1.07 (m, 4H).

### Example 18: 2-{[4-({2-[(4-chloro-2-fluorophenyl)methyl]pyrimidin-4-yl}oxy)piperidin-1-yl]methyl}-1-{[(2S)-oxetan-2-yl]methyl}-5-[(1E)-2-(pyridin-3-yl)ethenyl]-1H-1,3-benzodiazole (Compound 118a)

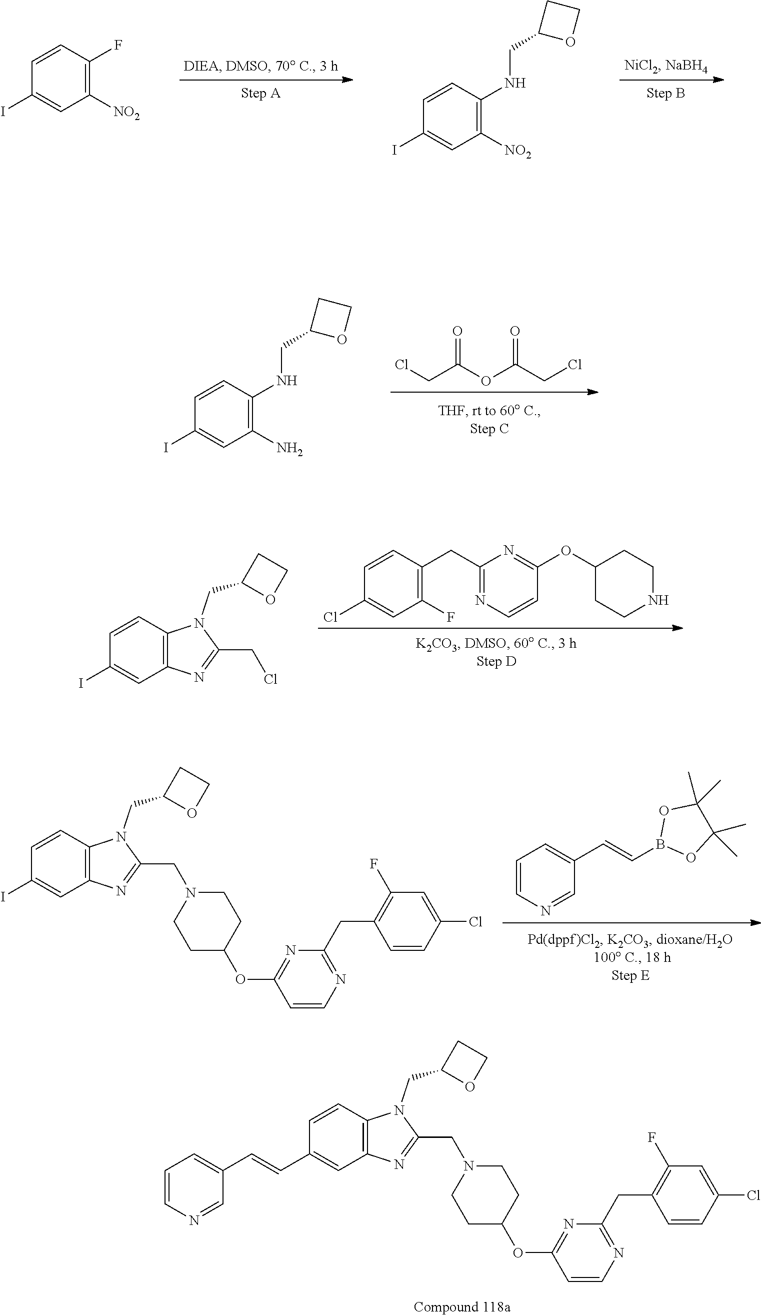

### Step A: (S)-4-iodo-2-nitro-N-(oxetan-2-ylmethyl)aniline

To a solution of 1-fluoro-4-iodo-2-nitrobenzene (1 g, 3.75 mmol) and (S)-oxetan-2-ylmethanamine MsOH salt (820 mg, 4.48 mmol) in DMSO (10 mL) was added DIEA (1.44 g, 11.2 mmol) at room temperature. The reaction was stirred at 70° C. for 3 hours. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography to give (S)-4-iodo-2-nitro-N-(oxetan-2-ylmethyl)aniline (1.3 g, 96% yield) as a yellow solid.

MS Calcd.: 334.0; MS Found: 334.9 [M+H]⁺.

### Step B: (S)-4-iodo-N1-(oxetan-2-ylmethyl)benzene-1,2-diamine

To a solution of (S)-4-iodo-2-nitro-N-(oxetan-2-ylmethyl)aniline (700 mg, 2.10 mmol) in MeOH (10 mL) was added NiCl₂ (543 mg, 4.20 mmol) at 0° C. Then NaBH₄ (238 mg, 6.3 mmol) was added slowly. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography to give (S)-4-iodo-N1-(oxetan-2-ylmethyl)benzene-1,2-diamine (540 mg, 85% yield) as a yellow solid.

MS Calcd.: 304.0; MS Found: 305.0 [M+H]⁺.

### Step C: (S)-2-(chloromethyl)-5-iodo-1-(oxetan-2-ylmethyl)-1H-benzo[d]imidazole

To a solution of (S)-4-iodo-N1-(oxetan-2-ylmethyl)benzene-1,2-diamine (440 mg, 1.45 mmol) in THE (5 mL) was added 2-chloroacetic anhydride (270 mg, 1.59 mmol). The reaction was stirred at room temperature for 1 hour. The reaction was monitored by LCMS which showed the starting material was consumed. Then the reaction was stirred at 60° C. for 16 hours. After the reaction was completed, the reaction was filtered and concentrated to give crude product which was purified by column chromatography to give (S)-2-(chloromethyl)-5-iodo-1-(oxetan-2-ylmethyl)-1H-benzo[d]imidazole (530 mg, crude, 80% purity) as yellow oil.

MS Calcd.: 362.0; MS Found: 362.8 [M+H]⁺.

### Step D: (S)-2-((4-((2-(4-chloro-2-fluorobenzyl)pyrimidin-4-yl)oxy)piperidin-1-yl)methyl)-5-iodo-1-(oxetan-2-ylmethyl)-1H-benzo[d]imidazole

A mixture of 2-(4-chloro-2-fluorobenzyl)-4-(piperidin-4-yloxy)pyrimidine (115 mg), (S)-2-(chloromethyl)-5-iodo-1-(oxetan-2-ylmethyl)-1H-benzo[d]imidazole (106 mg, 0.292 mmol) and K₂CO₃ (121 mg, 0.876 mmol) in DMSO (4.0 ml) was at 60° C. for 3 h. Upon cooling down, the mixture was diluted with H₂O (50 mL) and extracted with EA (15 mL×3). The combined organic layers were washed with brine (25 mL×1), dried over anhydrous Na₂SO₄ and concentrated under vacuum. The residue was purified by Prep-TLC to furnish (S)-2-((4-((2-(4-chloro-2-fluorobenzyl)pyrimidin-4-yl)oxy)piperidin-1-yl)methyl)-5-iodo-1-(oxetan-2-ylmethyl)-1H-benzo[d]imidazole (90 mg, yield: 47.6%) as a white solid.

MS Calcd.: 647.1; MS Found: 648.5 [M+H]⁺.

### Step E: 2-{[4-({2-[(4-chloro-2-fluorophenyl)methyl]pyrimidin-4-yl}oxy)piperidin-1-yl]methyl}-1-{[(2S)-oxetan-2-yl]methyl}-5-[(1E)-2-(pyridin-3-yl)ethenyl]-1H-1,3-benzodiazole (Compound 118a)

A mixture of (E)-3-(2-(4,4,5,5-tetramethyl-1,3,2-dioxaborolan-2-yl)vinyl)pyridine (48 mg, 0.209 mmol), Pd(dppf)Cl₂ (5.0 mg, 0.007 mmol), K₂CO₃ (29 mg, 0.209 mmol), H₂O (1.0 mL) and (S)-2-((4-((2-(4-chloro-2-fluorobenzyl) pyrimidin-4-yl)oxy)piperidin-1-yl)methyl)-5-iodo-1-(oxetan-2-ylmethyl)-1H-benzo[d]imidazole (45 mg, 0.070 mmol) in dioxane (4.0 ml) was stirred at 100° C. for 18 hours under Ar atmosphere. Upon cooling down, the solids were filtrated out and washed with EA. The filtrate was dried over anhydrous Na₂SO₄ and concentrated under vacuum. The residue was purified by Prep-HPLC (0.1% TFA) to furnish Compound 118a (8.3 mg) as a white solid.

MS Calcd.: 624.2; MS Found: 625.2 [M+H]⁺.

¹H NMR (400 MHz, MeOD): δ 9.01 (s, 1H), 8.79 (d, J=8.4 Hz, 1H), 8.66 (d, J=5.2 Hz, 1H), 8.44 (d, J=6.0 Hz, 1H), 8.02-7.98 (m, 2H), 7.81-7.70 (m, 3H), 7.42-7.32 (m, 2H), 7.22-7.15 (m, 2H), 6.80 (d, J=6.0 Hz, 1H), 5.31-5.19 (m, 2H), 4.79 (s, 2H), 4.76-4.60 (m, 3H), 4.47-4.40 (m, 1H), 4.21 (s, 2H), 3.62-3.45 (m, 4H), 2.84-2.75 (m, 1H), 2.56-2.47 (m, 1H), 2.23-2.03 (m, 4H).

### Example 19: N-(2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazol-5-yl)-6-phenylpyridine-3-carboxamide (Compound 119a)

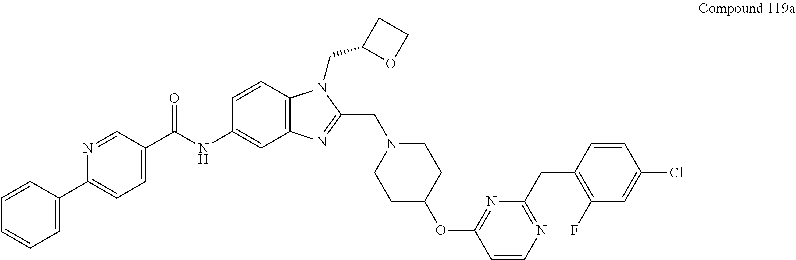 [SMILES: O=C(Nc1ccc2c(c1)nc(CN1CCC(Oc3ccnc(Cc4ccc(Cl)cc4F)n3)CC1)n2C[C@@H]1CCO1)c1ccc(-c2ccccc2)nc1]

The synthesis of Compound 119a was similar to that of Compound 111a except that 6-phenylnicotinic acid was used instead of pyridine-3-carboxylic acid. Compound 119a (30.47 mg, yield: 38%) was obtained as a white solid.

MS Calcd.: 717.3; MS Found: 718.4 [M+H]⁺.

¹H NMR (400 MHz, DMSO-d6): 10.43 (s, 1H), 9.22 (d, J=2.0 Hz, 1H), 8.40-8.44 (m, 2H), 8.13-8.21 (m, 3H), 8.08 (s, 1H), 7.50-7.62 (m, 5H), 7.38-7.43 (m, 2H), 7.25 (dd, J=8.0 Hz, 1.6 Hz, 1H), 6.74 (d, J=5.6 Hz, 1H), 5.06-5.13 (m, 1H), 4.85-4.93 (m, 1H), 4.64-4.72 (m, 1H), 4.47-4.59 (m, 2H), 4.33-4.41 (m, 1H), 4.15 (s, 2H), 3.89 (d, J=13.6 Hz, 1H), 3.75 (d, J=13.2 Hz, 1H), 2.65-2.83 (m, 3H), 2.38-2.49 (m, 1H), 2.20-2.32 (m, 2H), 1.83-1.94 (m, 2H), 1.53-1.66 (m, 2H).

### Example 20: 2-[(4-chlorophenyl)methyl]-4-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyrimidine (Compound 120a)

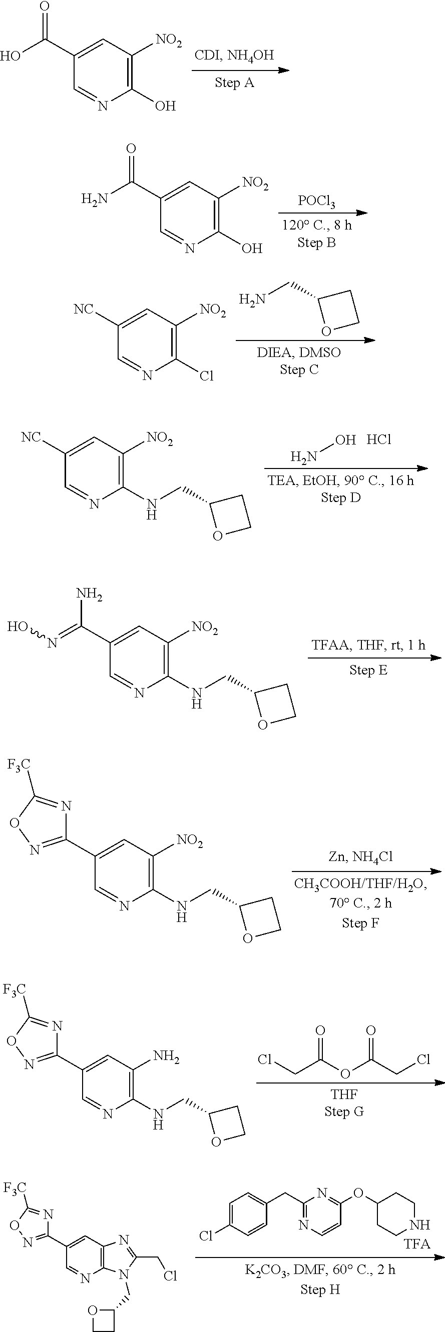

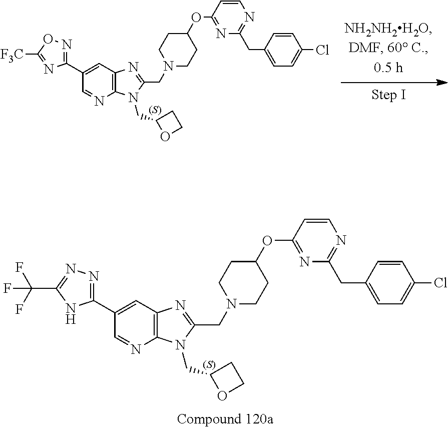

### Step A: 6-hydroxy-5-nitronicotinamide

To a solution of 6-hydroxy-5-nitronicotinic acid (5 g, 27.2 mmol) in DMF (25 mL) was added CDI (4.85 g, 29.9 mmol) at room temperature. The reaction was stirred at 50° C. for 2 hours. Then ammonia (35 mL, 25%-28% w/w aqueous solution) was added and the reaction was stirred at room temperature for 1 h. The reaction mixture was filtered and washed with water. The solid was dried to afford 6-hydroxy-5-nitronicotinamide (4.2 g, crude 86% yield) as a yellow solid.

### Step B: 6-chloro-5-nitronicotinonitrile

6-hydroxy-5-nitronicotinamide (4.2 g, 23.0 mmol) was dissolved in POCl₃ (40 mL). The reaction was stirred at 120° C. for 8 hours. The reaction was concentrated under reduced pressure to give crude product. The crude product was dissolved in ethyl acetate (100 mL) and washed with NaHCO₃ solution. The organic layer was washed with brine (50 mL×2), dried over sodium sulfate, filtered and concentrated in vacuum to give crude product. The crude product was purified by column chromatography (PE:EA=10:1) to afford 6-chloro-5-nitronicotinonitrile (2.7 g, 64% yield) as yellow solid.

### Step C: (S)-5-nitro-6-((oxetan-2-ylmethyl)amino)nicotinonitrile

To a solution of 6-chloro-5-nitronicotinonitrile (1 g, 5.5 mmol) and (S)-oxetan-2-ylmethanamine MsOH salt (1.2 g, 6.5 mmol) in DMSO (10 mL) was added DIEA (2.11 g, 16.5 mmol) at room temperature. The reaction was stirred at 70° C. for 1 hour. After the reaction was completed, the reaction was quenched with water (50 mL) and extracted with ethyl acetate (100 mL×3). The organic layer was combined and washed with brine (100 mL×2), dried over sodium sulfate, filtered and concentrated in vacuum. The residue was purified by column chromatography (PE:EA=3:1) to give (S)-3-nitro-4-((oxetan-2-ylmethyl)amino) benzonitrile (1.4 g, crude) as an orange solid.

MS Calcd.: 234.1; MS Found: 235.1 [M+H]⁺.

### Step D: (S)—N′-hydroxy-5-nitro-6-((oxetan-2-ylmethyl)amino)nicotinimidamide

To a solution of (S)-3-nitro-4-((oxetan-2-ylmethyl)amino) benzonitrile (1.4 g, 6.0 mmol) in EtOH (15 mL) was added Hydroxylamine hydrochloride (2.06 g, 30.0 mmol) and TEA (3.6 g, 36.0 mmol). The reaction was stirred at 90° C. for 16 hours. After the reaction was completed, the reaction was quenched with water (50 mL) and extracted with ethyl acetate (100 mL×3). The organic layers were combined and washed with brine (50 mL×2), dried over sodium sulfate, filtered and concentrated in vacuum. The residue was purified by column chromatography (DCM:MeOH=10:1) to give (S)—N′-hydroxy-5-nitro-6-((oxetan-2-ylmethyl)amino)nicotinimidamide (1.1 g, 69% yield) as yellow solid.

MS Calcd.: 267.1; MS Found: 268.1 [M+H]⁺.

### Step E: (S)-3-nitro-N-(oxetan-2-ylmethyl)-5-(5-(trifluoromethyl)-1,2,4-oxadiazol-3-yl)pyri dine-2-amine

To a solution of (S)—N′-hydroxy-5-nitro-6-((oxetan-2-ylmethyl)amino) nicotinimidamide (1.0 g, 3.7 mmol) in THE (10 mL) was added TFAA (3.14 g, 14.8 mmol) at room temperature. The reaction was stirred at room temperature for 1 hours. After the reaction was completed, the reaction was quenched with NaHCO₃ solution (50 mL) and extracted with ethyl acetate (100 mL×3). The organic layer was combined and washed with brine (50 mL×2), dried over sodium sulfate, filtered and concentrated in vacuum. The residue was purified by column chromatography (PE:EA=5:1) to give (S)-3-nitro-N-(oxetan-2-ylmethyl)-5-(5-(trifluoromethyl)-1,2,4-oxadiazol-3-yl)pyridine-2-amine (1.0 g, 76% yield) as yellow solid.

MS Calcd.: 345.1; MS Found: 346.1 [M+H]⁺.

### Step F: (S)—N2-(oxetan-2-ylmethyl)-5-(5-(trifluoromethyl)-1,2,4-oxadiazol-3-yl)pyridine-2,3-diamine

To a solution of (S)-3-nitro-N-(oxetan-2-ylmethyl)-5-(5-(trifluoromethyl)-1,2,4-oxadiazol-3-yl)pyridine-2-amine (950 mg, 2.75 mmol) in THF/H₂O (10 mL/1 mL) was added Zn (1.8 g, 27.5 mmol), NH₄Cl (2.9 g, 55.0 mmol) and CH₃COOH (0.5 ml). The reaction was stirred at 70° C. for 2 hours. After the reaction was completed, the reaction was filtered and concentrated in vacuum. The residue was purified by column chromatography (PE:EA=1:1) to give (S)—N2-(oxetan-2-ylmethyl)-5-(5-(trifluoromethyl)-1,2,4-oxadiazol-3-yl)pyridine-2,3-diamine (600 mg, 69% yield) as yellow solid.

MS Calcd.: 315.1; MS Found: 316.1 [M+H]⁺.

### Step G: (S)-3-(2-(chloromethyl)-3-(oxetan-2-ylmethyl)-3H-imidazo[4,5-b]pyridin-6-yl)-5-(trifluoromethyl)-1,2,4-oxadiazole

To a solution of (S)—N2-(oxetan-2-ylmethyl)-5-(5-(trifluoromethyl)-1,2,4-oxadiazol-3-yl)pyridine-2,3-diamine (600 mg, 1.9 mmol) in THE (6 mL) was added 2-chloroacetic anhydride (356 mg, 2.1 mmol) at room temperature. The reaction was stirred at room temperature for 1 hour. Then the reaction was stirred at 60° C. for 16 hours. After the reaction was completed, the reaction was concentrated in vacuum. The residue was purified by column chromatography (PE:EA=2:1) to give (S)-3-(2-(chloromethyl)-3-(oxetan-2-ylmethyl)-3H-imidazo[4,5-b]pyridin-6-yl)-5-(trifluoromethyl)-1,2,4-oxadiazole (440 mg, 62% yield) as yellow solid.

MS Calcd.: 373.1; MS Found: 374.0 [M+H]⁺.

### Step H: (S)-3-(2-((4-((2-(4-chlorobenzyl)pyrimidin-4-yl)oxy)piperidin-1-yl)methyl)-3-(oxetan-2-ylmethyl)-3H-imidazo[4,5-b]pyridin-6-yl)-5-(trifluoromethyl)-1,2,4-oxadiazole

To a solution of 2-(4-chlorobenzyl)-4-(piperidin-4-yloxy)pyrimidine (145 mg) in DMF (2 mL) was added K₂CO₃ (111 mg, 0.804 mmol). After a few minutes, (S)-3-(2-(chloromethyl)-3-(oxetan-2-ylmethyl)-3H-imidazo[4,5-b]pyridin-6-yl)-5-(trifluoromethyl)-1,2,4-oxadiazole (100 mg, 0.268 mmol) was added. The resulting mixture was heated to 60° C. for 2 hours. The reaction was quenched with water, extracted with ethyl acetate. The organic layers were combined, dried over Na₂SO₄, filtered and concentrated under vacuum. The residue was purified by silica gel column and eluted with DCM:MeOH=20:1 to give (S)-3-(2-((4-((2-(4-chlorobenzyl)pyrimidin-4-yl)oxy)piperidin-1-yl)methyl)-3-(oxetan-2-ylmethyl)-3H-imidazo[4,5-b]pyridin-6-yl)-5-(trifluoromethyl)-1,2,4-oxadiazole (100 mg, 58.5% yield) as a white solid.

MS Calcd.: 640.2; MS Found: 641.3 [M+H]⁺.

### Step I: 2-[(4-chlorophenyl)methyl]-4-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyrimidine (Compound 120a)

To a solution of (S)-3-(2-((4-((2-(4-chlorobenzyl)pyrimidin-4-yl)oxy)piperidin-1-yl)methyl)-3-(oxetan-2-ylmethyl)-3H-imidazo[4,5-b]pyridin-6-yl)-5-(trifluoromethyl)-1,2,4-oxadiazole (100 mg, 0.156 mmol) in DMF (2 mL) was added hydrazine hydrate (0.1 mL, 85% wt.). The solution was heated to 60° C. for 30 minutes. The reaction mixture was filtered, the filtrate was purified by Prep-HPLC to afford Compound 120a (18.6 mg, yield: 18.6%) as a white solid.

MS Calcd.: 639.2; MS Found: 640.3 [M+H]⁺.

¹H NMR (400 MHz, DMSO-d₆): δ 15.42 (brs, 1H), 9.00 (d, J=2.0 Hz, 1H), 8.61 (d, J=1.6 Hz, 1H), 8.42 (d, J=6.0 Hz, 1H), 7.31-7.36 (m, 4H), 6.73 (d, J=6.0 Hz, 1H), 5.14-5.21 (m, 1H), 5.01-5.10 (m, 1H), 4.80-4.88 (m, 1H), 4.68-4.74 (m, 1H), 4.47-4.53 (m, 1H), 4.32-4.39 (m, 1H), 4.08 (s, 2H), 3.92-4.06 (m, 2H), 2.66-2.88 (m, 3H), 2.34-2.55 (m, 3H), 1.91-2.01 (m, 2H), 1.61-1.74 (m, 2H).

### Example 21: 2-[(4-chlorophenyl)methyl]-5-fluoro-4-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyrimidine (Compound 121a)

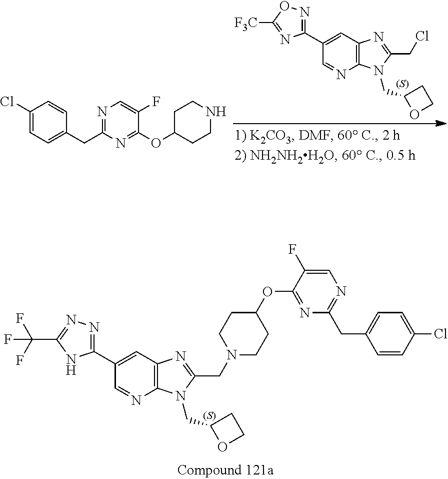

A mixture of 2-(4-chlorobenzyl)-5-fluoro-4-(piperidin-4-yloxy)pyrimidine (223 mg), (S)-3-(2-(chloromethyl)-3-(oxetan-2-ylmethyl)-3H-imidazo[4,5-b]pyridin-6-yl)-5-(trifluoromethyl)-1,2,4-oxadiazole (160 mg, 0.429 mmol) and K₂CO₃ (177.6 mg, 1.287 mmol) in DMF (3 mL) was heated to 60° C. for 2 hours. Then hydrazine hydrate (0.1 mL, 85% wt.) was added. The reaction mixture was stirred at 60° C. for 30 minutes. The reaction mixture was filtered, the filtrate was purified by Prep-HPLC to afford Compound 121a (32.33 mg, yield: 11.4%) as a white solid.

MS Calcd.: 657.2; MS Found: 658.3 [M+H]⁺.

¹H NMR (400 MHz, DMSO-d₆): δ 8.97 (d, J=2.0 Hz, 1H), 8.50 (d, J=2.8 Hz, 1H), 8.41 (d, J=1.6 Hz, 1H), 7.31-7.37 (m, 4H), 5.07-5.20 (m, 2H), 4.76-4.81 (m, 1H), 4.63-4.71 (m, 1H), 4.45-4.53 (m, 1H), 4.34-4.40 (m, 1H), 4.09 (s, 2H), 3.88-3.96 (m, 2H), 2.65-2.82 (m, 3H), 2.47-2.54 (m, 1H), 2.38-2.45 (m, 2H), 1.93-2.02 (m, 2H), 1.65-1.77 (m, 2H).

### Example 22: 2-{[4-({2-[(4-chlorophenyl)methyl]pyrimidin-4-yl}oxy)piperidin-1-yl]methyl}-6-fluoro-1-{[(2S)-oxetan-2-yl]methyl}-5-[5-(trifluoromethyl)-4H-1,2,4-triazol-3-yl]-1H-1,3-benzodiazole (Compound 122a)

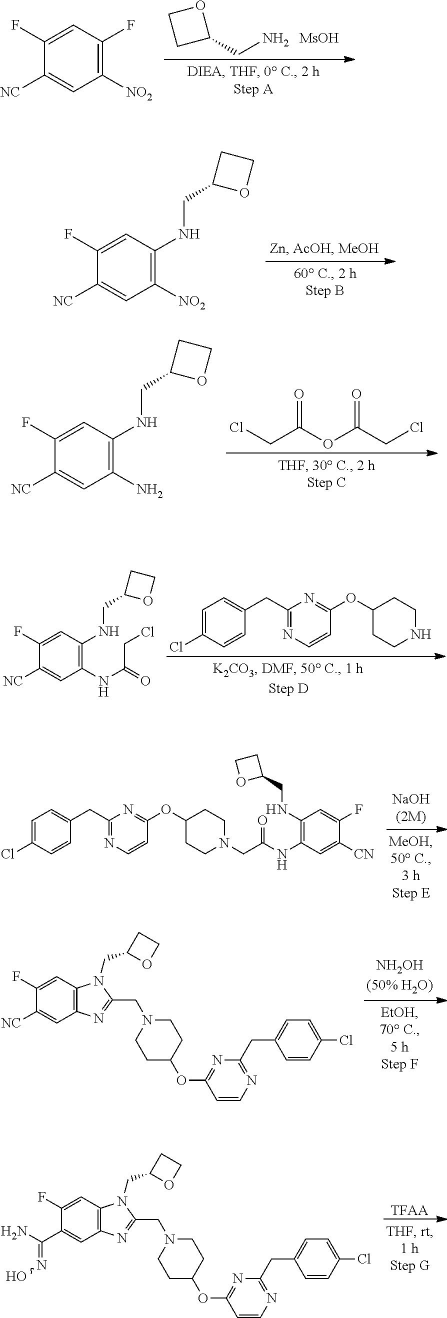

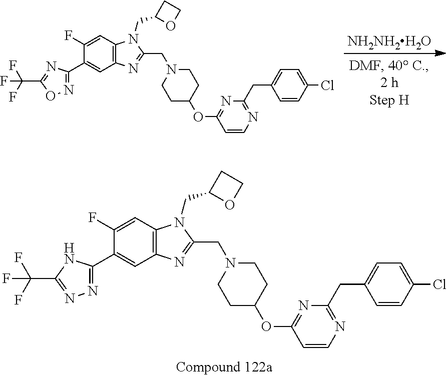 [SMILES: Fc1cc2c(cc1-c1noc(C(F)(F)F)n1)nc(CN1CCC(Oc3ccnc(Cc4ccc(Cl)cc4)n3)CC1)n2C[C@@H]1CCO1 ; "NH2NH2·H2O" ; Fc1cc2c(cc1-c1nnc(C(F)(F)F)[nH]1)nc(CN1CCC(Oc3ccnc(Cc4ccc(Cl)cc4)n3)CC1)n2C[C@@H]1CCO1]

### Step A: (S)-2-fluoro-5-nitro-4-((oxetan-2-ylmethyl)amino)benzonitrile

To a solution 2,4-difluoro-5-nitrobenzonitrile (3 g, 6.3 mmol) in THF (30 mL) was added (S)-oxetan-2-ylmethanamine MsOH salt (2.69 g, 14.7 mmol) and DIEA (6 g, 49 mmol) successively at 0° C. The reaction mixture was stirred at 0° C. for 2 hours. After the reaction was completed, the reaction was quenched with H₂O and extracted with EA (30 mL*3). The organic layers were combined, dried over Na₂SO₄ and concentrated. The residue was purified by silica gel column chromatography (PE/EA=1/1) to give (S)-2-fluoro-5-nitro-4-((oxetan-2-ylmethyl)amino) benzonitrile (2.1 g, 70% yield) as a yellow solid.

MS Calcd.: 251.1; MS Found: 251.9 [M+H]⁺.

### Step B: (S)-5-amino-2-fluoro-4-((oxetan-2-ylmethyl)amino)benzonitrile

To a solution of (S)-2-fluoro-5-nitro-4-((oxetan-2-ylmethyl)amino)benzonitrile (2.1 g, 8.4 mmol) and Zn (5.5 g) in MeOH (21 mL) was added AcOH (1.51 g) at room temperature. The reaction mixture was stirred at 60° C. for 2 hours. After the reaction was completed, the reaction was filtered and the solid was washed with NaHCO₃ (aq). The filtrate was quenched with H₂O and extracted with EA (20 mL*3). The organic layers were combined, dried over Na₂SO₄ and concentrated to give (S)-5-amino-2-fluoro-4-((oxetan-2-ylmethyl)amino)benzonitrile (1.8 g, 85.7% yield) as a yellow solid.

MS Calcd.: 221.1; MS Found: 222.1 [M+H]⁺.

### Step C: (S)-2-chloro-N-(5-cyano-4-fluoro-2-((oxetan-2-ylmethyl)amino)phenyl) acetamide

To a solution of (S)-5-amino-2-fluoro-4-((oxetan-2-ylmethyl)amino) benzonitrile (1.8 g, 8.14 mmol) in THE (18 mL) was added 2-chloroacetic anhydride (1.7 g, 9.77 mmol). The reaction was stirred at 30° C. for 2 hours. After the reaction was completed, the reaction mixture was concentrated and the residue was purified by silica gel column chromatography (DCM/MeOH=10/1) to give (S)-2-chloro-N-(5-cyano-4-fluoro-2-((oxetan-2-ylmethyl)amino)phenyl) acetamide (610 mg, 30% yield) as yellow oil.

MS Calcd.: 297.1; MS Found: 298.1 [M+H]⁺.

### Step D: (S)-2-(4-((2-(4-chlorobenzyl)pyrimidin-4-yl)oxy)piperidin-1-yl)-N-(5-cyano-4-fluoro-2-((oxetan-2-ylmethyl)amino)phenyl)acetamide

To a mixture of 2-(4-chlorobenzyl)-4-(piperidin-4-yloxy)pyrimidine (1.2 g, 3.83 mmol) in DMF (20 mL) was added K₂CO₃ (1.6 g, 11.49 mmol), (S)-2-chloro-N-(5-cyano-4-fluoro-2-((oxetan-2-ylmethyl)amino)phenyl)acetamide (910 mg, 3.06 mmol) and the mixture was heated to 50° C. for 1 hour. The mixture filtered and the filtrate was concentrated in vacuum. The residue was purified by silica gel column (DCM/MeOH=30/1) to give (S)-2-(4-((2-(4-chlorobenzyl)pyrimidin-4-yl)oxy)piperidin-1-yl)-N-(5-cyano-4-fluoro-2-((oxetan-2-ylmethyl) amino)phenyl)acetamide (1.42 g, yield: 80%) as a yellow solid.

MS Calcd.: 564.2; MS Found: 565.2 [M+H]⁺.

### Step E: The synthesis of (S)-2-((4-((2-(4-chlorobenzyl)pyrimidin-4-yl)oxy)piperidin-1-yl)methyl)-6-fluoro-1-(oxetan-2-ylmethyl)-1H-benzo[d]imidazole-5-carbonitrile

To a solution of (S)-2-(4-((2-(4-chlorobenzyl)pyrimidin-4-yl)oxy)piperidin-1-yl)-N-(5-cyano-4-fluoro-2-((oxetan-2-ylmethyl)amino)phenyl)acetamide (1.3 g, 2.3 mmol) in MeOH (20 mL) was added 2 M NaOH (4.6 mL) and the mixture solution was stirred at 50° C. for 3 hours. The reaction was quenched with water and extracted with DCM/MeOH (20/1), The organic layers were combined and dried over Na₂SO₄, filtered and concentrated under vacuum. The residue was purified by a silica gel column (DCM/MeOH=30/1) to give (S)-2-((4-((2-(4-chlorobenzyl)pyrimidin-4-yl)oxy)piperidin-1-yl)methyl)-6-fluoro-1-(oxetan-2-ylmethyl)-1H-benzo[d]imidazole-5-carbonitrile (810 mg, yield: 64%) as a yellow solid.

MS Calcd.: 546.2; MS Found: 547.2 [M+H]⁺.

### Step F: (S)-2-((4-((2-(4-chlorobenzyl)pyrimidin-4-yl)oxy)piperidin-1-yl)methyl)-6-fluoro-N′-hydroxy-1-(oxetan-2-ylmethyl)-1H-benzo[d]imidazole-5-carboximidamide

To a solution of (S)-2-((4-((2-(4-chlorobenzyl)pyrimidin-4-yl)oxy)piperidin-1-yl)methyl)-6-fluoro-1-(oxetan-2-ylmethyl)-1H-benzo[d]imidazole-5-carbonitrile (760 mg, 1.4 mmol) in EtOH (10 mL) was added NH₂OH (50% in H₂O) (230 mg, 7.0 mmol) and The resulting mixture was stirred at 70° C. for 5 hours. The reaction was quenched with water, extracted with DCM. The organic layers were combined, dried over Na₂SO₄, filtered and concentrated to afford (S)-2-((4-((2-(4-chlorobenzyl)pyrimidin-4-yl)oxy)piperidin-1-yl)methyl)-6-fluoro-N′-hydroxy-1-(oxetan-2-ylmethyl)-1H-benzo[d]imidazole-5-carboximidamide (810 mg) as a white solid. The crude product was used for the next step without further purification.

MS Calcd.: 579.2; MS Found: 580.2 [M+H]⁺.

### Step G: (S)-3-(2-((4-((2-(4-chlorobenzyl)pyrimidin-4-yl)oxy)piperidin-1-yl)methyl)-6-fluoro-1-(oxetan-2-ylmethyl)-1H-benzo[d]imidazol-5-yl)-5-(trifluoromethyl)-1,2,4-oxadiazole

To a solution of (S)-2-((4-((2-(4-chlorobenzyl)pyrimidin-4-yl)oxy)piperidin-1-yl)methyl)-6-fluoro-N′-hydroxy-1-(oxetan-2-ylmethyl)-1H-benzo[d]imidazole-5-carboximidamide (400 mg) in THE (8.0 mL) was added TFAA (435 mg, 2.07 mmol) and The resulting mixture was stirred at rt for 1 hour. The reaction was quenched with water and extracted with DCM/MeOH (20/1). The organic layers were combined and dried over Na₂SO₄, filtered and concentrated under vacuum. The residue was purified by silica gel column (DCM/MeOH=30/1) to give (S)-3-(2-((4-((2-(4-chlorobenzyl)pyrimidin-4-yl)oxy)piperidin-1-yl)methyl)-6-fluoro-1-(oxetan-2-ylmethyl)-1H-benzo[d]imidazol-5-yl)-5-(trifluoromethyl)-1,2,4-oxadiazole (400 mg, yield: 88%) as a white solid.

MS Calcd.: 657.2; MS Found: 658.2 [M+H]⁺.

### Step H: 2-{[4-({2-[(4-chlorophenyl)methyl]pyrimidin-4-yl}oxy)piperidin-1-yl]methyl}-6-fluoro-1-{[(2S)-oxetan-2-yl]methyl}-5-[5-(trifluoromethyl)-4H-1,2,4-triazol-3-yl]-1H-1,3-benzodiazole (Compound 122a)

To a solution of (S)-3-(2-((4-((2-(4-chlorobenzyl)pyrimidin-4-yl)oxy)piperidin-1-yl)methyl)-6-fluoro-1-(oxetan-2-ylmethyl)-1H-benzo[d]imidazol-5-yl)-5-(trifluoromethyl)-1,2,4-oxadiazole (170 mg, 0.26 mmol) in DMF (3 ml) was added NH₂NH₂·H₂O (26 mg, 0.52 mmol) and the resulting mixture was stirred at 40° C. for 2 hours. The mixture was purified by Prep-HPLC to give 2-{[4-({2-[(4-chlorophenyl)methyl]pyrimidin-4-yl}oxy)piperidin-1-yl]methyl}-6-fluoro-1-{[(2S)-oxetan-2-yl]methyl}-5-[5-(trifluoromethyl)-4H-1,2,4-triazol-3-yl]-1H-1,3-benzodiazole (37.37 mg, yield: 22%) as white solid.

MS Calcd.: 656.2; MS Found: 657.3 [M+H]⁺.

¹H NMR (400 MHz, DMSO-d₆): 8.41 (d, J=5.6 Hz, 1H), 8.08 (d, J=6.4 Hz, 1H), 7.66 (d, J=7.2 Hz, 1H), 7.30-7.37 (m, 4H), 6.73 (d, J=6.0 Hz, 1H), 5.07-5.13 (m, 1H), 4.98-5.07 (m, 1H), 4.69-4.76 (m, 1H), 4.48-4.60 (m, 2H), 4.39-4.44 (m, 1H), 4.08 (s, 2H), 3.91 (d, J=14.0 Hz, 1H), 3.77 (d, J=13.6 Hz, 1H), 2.66-2.81 (m, 3H), 2.30-2.49 (m, 3H), 1.89-1.97 (m, 2H), 1.58-1.70 (m, 2H).

### Example 23: 2-[(4-chlorophenyl)methyl]-4-[(1-{[3-(2-ethoxyethyl)-6-[5-(trifluoromethyl)-4H-1,2,4-triazol-3-yl]-3H-imidazo[4,5-b]pyridin-2-yl]methyl}piperidin-4-yl)oxy]pyrimidine (Compound 123)

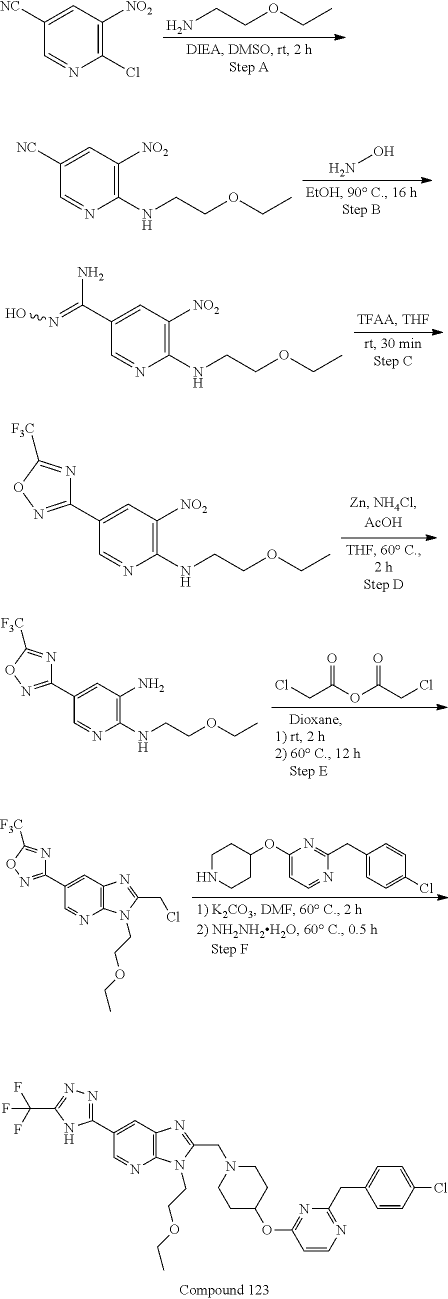

### Step A: 6-((2-ethoxyethyl)amino)-5-nitronicotinonitrile

To a solution 6-chloro-5-nitronicotinonitrile (1.0 g, 5.45 mmol) and 2-ethoxyethan-1-amine (514 mg, 5.78 mmol) in DMSO (15 mL) was added DIEA (2.1 g, 16.38 mmol). The reaction mixture was stirred at rt for 2 hours. After the reaction was completed, the reaction was quenched with H₂O and extracted with EA (20 mL*3). The organic layers were combined, dried over Na₂SO₄ and concentrated. The residue was purified by silica gel column chromatography (PE/EA=2/1) to give 6-((2-ethoxyethyl)amino)-5-nitronicotinonitrile (1.1 g, 73% yield) as a yellow solid.

MS Calcd.: 236.1; MS Found: 237.1 [M+H]⁺.

### Step B: 6-((2-ethoxyethyl)amino)-N′-hydroxy-5-nitronicotinimidamide

To a solution of 6-((2-ethoxyethyl)amino)-5-nitronicotinonitrile (1 g, 4.2 mmol) and NH₂OH·HCl (1.75 g, 25.2 mmol) in EtOH (10 mL) was added TEA (4.2 g 42 mmol) at room temperature. The reaction mixture was stirred at 90° C. for 16 hours. After the reaction was completed, the reaction was quenched with H₂O and extracted with EA (20 mL*3). The organic layers were combined, dried over Na₂SO₄ and concentrated to give 6-((2-ethoxyethyl)amino)-N′-hydroxy-5-nitronicotinimidamide (1 g, 88% yield) as a yellow solid.

MS Calcd.: 269.1; MS Found: 270.1 [M+H]⁺.

### Step C: N-(2-ethoxyethyl)-3-nitro-5-(5-(trifluoromethyl)-1,2,4-oxadiazol-3-yl)pyridin-2-amine

To a solution of 6-((2-ethoxyethyl)amino)-N′-hydroxy-5-nitronicotinimidamide (1 g, 3.7 mmol) and TFAA (3.9 g, 18.6 mmol) in THF (10 mL). The reaction was stirred at rt for 30 min. After the reaction was completed, the residue was quenched with H₂O and extracted with EA (20 mL*3). The organic layers were combined, dried over Na₂SO₄ and concentrated. The residue was purified by silica gel column chromatography (PE/EA=10/1) to give N-(2-ethoxyethyl)-3-nitro-5-(5-(trifluoromethyl)-1,2,4-oxadiazol-3-yl)pyridin-2-amine (1.3 g, 95% yield) as a yellow solid.

MS Calcd.: 347.1; MS Found: 348.1 [M+H]⁺.

### Step D: N2-(2-ethoxyethyl)-5-(5-(trifluoromethyl)-1,2,4-oxadiazol-3-yl)pyridine-2,3-diamine

A mixture of N-(2-ethoxyethyl)-3-nitro-5-(5-(trifluoromethyl)-1,2,4-oxadiazol-3-yl)pyridin-2-amine (1.2 g, 3.46 mmol) and Zn (1.12 g, 17.3 mmol) in THE (12 mL) was added in NH₄Cl (1.85 g, 34.6 mmol) and 6 drop of AcOH. The reaction was stirred at 60° C. for 2 hours. After the reaction was completed, the reaction was quenched with H₂O and extracted with EA (15 mL*3). The organic layers were combined, dried over Na₂SO₄ and concentrated to give N2-(2-ethoxyethyl)-5-(5-(trifluoromethyl)-1,2,4-oxadiazol-3-yl)pyridine-2,3-diamine (1 g, 83% yield) as a brown solid.

MS Calcd.: 317.1; MS Found: 318.1 [M+H]+

### Step E: 3-(2-(chloromethyl)-3-(2-ethoxyethyl)-3H-imidazo[4,5-b]pyridin-6-yl)-5-(trifluoromethyl)-1,2,4-oxadiazole

To a solution of N2-(2-ethoxyethyl)-5-(5-(trifluoromethyl)-1,2,4-oxadiazol-3-yl)pyridine-2,3-diamine (500 mg) in Dioxane (5 mL) was added 2-chloroacetic anhydride (323 mg, 1.89 mmol). The reaction was stirred at rt for 2 hours. After the reaction was completed, the reaction was stirred at 60° C. for 12 hours. The reaction was quenched with H₂O and extracted with EA (5 mL*3). The organic layers were combined, and concentrated. The residue was purified by silica gel column chromatography (PE/EA=5/1) to give 3-(2-(chloromethyl)-3-(2-ethoxyethyl)-3H-imidazo[4,5-b]pyridin-6-yl)-5-(trifluoromethyl)-1,2,4-oxadiazole (630 mg, yield: 100%) as yellow oil.

MS Calcd.: 375.1; MS Found: 376.1[M+H]+.

### Step F: 2-[(4-chlorophenyl)methyl]-4-[(1-{[3-(2-ethoxyethyl)-6-[5-(trifluoromethyl)-4H-1,2,4-triazol-3-yl]-3H-imidazo[4,5-b]pyridin-2-yl]methyl}piperidin-4-yl)oxy]pyrimidine (Compound 123)

To a solution of 2-(4-chlorobenzyl)-4-(piperidin-4-yloxy)pyrimidine (320 mg, 1.06 mmol) and K₂CO₃ (220 mg, 1.59 mmol) in DMF (2 mL) was added 3-(2-(chloromethyl)-3-(2-ethoxyethyl)-3H-imidazo[4,5-b]pyridin-6-yl)-5-(trifluoromethyl)-1,2,4-oxadiazole (200 mg, 0.53 mmol). The reaction was stirred at 60° C. for 2 h. NH₂NH₂·H₂O (80 mg, 1.59 mmol) was added and the mixture was stirred at 60° C. for 0.5 h. The reaction mixture was purified by Prep-HPLC to give Compound 123 (7.85 mg, yield: 2%) as a white solid.

MS Calcd.: 641.2; MS Found: 642.4 [M+H]⁺.

¹H NMR (400 MHz, DMSO-d₆): δ 8.99 (d, J=2.0 Hz, 1H), 8.57 (d, J=2.0 Hz, 1H), 8.41 (d, J=6.0 Hz, 1H), 7.31-7.36 (m, 4H), 6.73 (d, J=5.6 Hz, 1H), 4.98-5.06 (m, 1H), 4.63 (t, J=5.6 Hz, 2H), 4.08 (s, 2H), 3.92 (s, 2H), 3.81 (t, J=5.6 Hz, 2H), 3.40-3.47 (m, 2H), 2.70-2.79 (m, 2H), 2.30-2.43 (m, 2H), 1.91-1.98 (m, 2H), 1.60-1.70 (m, 2H), 1.04 (t, J=6.8 Hz, 3H).

### Example 24: 3-(2-{[4-({2-[(4-chlorophenyl)methyl]pyrimidin-4-yl}oxy)piperidin-1-yl]methyl}-3-{[(2S)-oxetan-2-yl]methyl}-3H-imidazo[4,5-b]pyridin-6-yl)-4,5-dihydro-1,2,4-oxadiazol-5-one (Compound 144a)

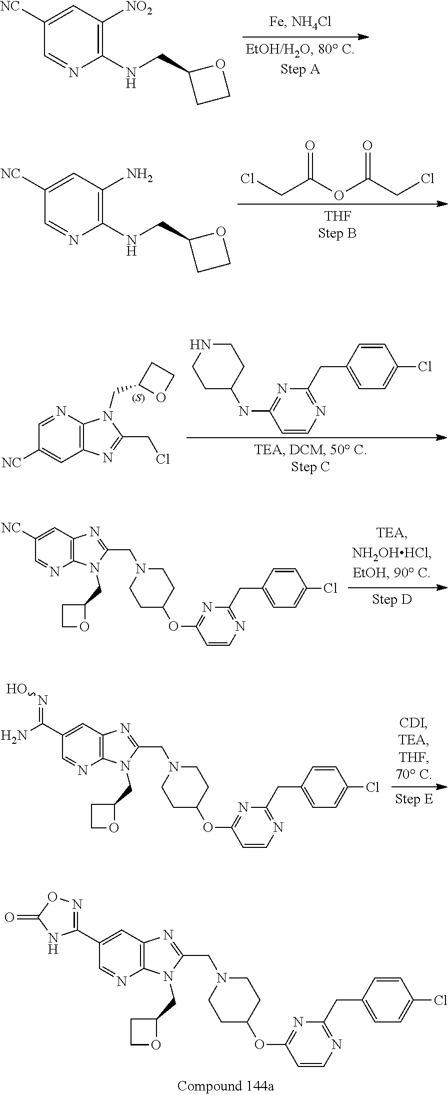

### Step A: (S)-5-amino-6-((oxetan-2-ylmethyl)amino)nicotinonitrile

A mixture of (S)-5-nitro-6-((oxetan-2-ylmethyl)amino)nicotinonitrile (3.0 g, 12.8 mmol), Fe (3.6 g, 64.1 mmol) and NH₄Cl (6.9 g, 128.2 mmol) in EtOH (60 mL) and water (20 mL) was stirred at 80° C. for 1 hour. The mixture was poured into cold water (200 mL) and extracted with EtOAc (2×200 mL), the combined organic layer was washed with brine, dried over sodium sulfate, filtered and the residue was concentrated to obtain (S)-5-amino-6-((oxetan-2-ylmethyl)amino)nicotinonitrile (2.4 g, yield: 91%) as white solid. MS Calcd: 204.1; MS Found: 205.3 [M+H]⁺.

### Step B: (S)-2-(chloromethyl)-3-(oxetan-2-ylmethyl)-3H-imidazo[4,5-b]pyridine-6-carbonitrile

To a solution of (S)-5-amino-6-((oxetan-2-ylmethyl)amino)nicotinonitrile (2.4 g) in THF (20 mL) was added 2-chloroacetic anhydride (2.0 g, 11.7 mmol). The mixture was stirred at room temperature for 2 hours. Then the mixture was stirred at 60° C. for 16 hours. Then, another portion of 2-chloroacetic anhydride was added, the mixture was stirred at 60° C. for another 16 h. The mixture was poured into sodium bicarbonate aqueous solution (200 mL) and extracted with EtOAc (2×200 mL), the combined organic layer was washed with brine, dried over sodium sulfate, filtered and the residue was purified by column chromatography on silica gel to obtain (S)-2-(chloromethyl)-3-(oxetan-2-ylmethyl)-3H-imidazo[4,5-b]pyridine-6-carbonitrile (1.1 g, yield: 36%) as yellow oil. MS Calcd: 262.1; MS Found: 263.1 [M+H]⁺.

### Step C: (S)-2-((4-((2-(4-chlorobenzyl)pyrimidin-4-yl)oxy)piperidin-1-yl)methyl)-3-(oxetan-2-ylmethyl)-3H-imidazo[4,5-b]pyridine-6-carbonitrile

To a solution of 2-(4-chlorobenzyl)-4-(piperidin-4-yloxy)pyrimidine (526 mg, 1.7 mmol) in DMF (5 mL) was added TEA (345 mg, 3.4 mmol) and (S)-2-(chloromethyl)-3-(oxetan-2-ylmethyl)-3H-imidazo[4,5-b]pyridine-6-carbonitrile (150 mg, 0.57 mmol). The resulting mixture was stirred at 50° C. for 2 hours. After the reaction was completed, the mixture was diluted with EA (50 mL), washed with H₂O (40 mL). The organic layer was dried over anhydrous sodium sulfate, filtered, and evaporated to dryness. The residue was purified by column chromatography on silica gel (DCM/MeOH=1/50) to give (S)-2-((4-((2-(4-chlorobenzyl)pyrimidin-4-yl)oxy)piperidin-1-yl)methyl)-3-(oxetan-2-ylmethyl)-3H-imidazo[4,5-b]pyridine-6-carbonitrile (200 mg, yield: 66%) as yellow oil. MS Calcd.: 529.2; MS Found: 530.2 [M+H]⁺.

### Step D: (S)-2-((4-((2-(4-chlorobenzyl)pyrimidin-4-yl)oxy)piperidin-1-yl)methyl)-N′-hydroxy-3-(oxetan-2-ylmethyl)-3H-imidazo[4,5-b]pyridine-6-carboximidamide

To a solution of (S)-2-((4-((2-(4-chlorobenzyl)pyrimidin-4-yl)oxy)piperidin-1-yl)methyl)-3-(oxetan-2-ylmethyl)-3H-imidazo[4,5-b]pyridine-6-carbonitrile (150 mg, 0.3 mmol) in EtOH (4 mL) was added TEA (114 mg, 1.1 mmol) and NH₂OH·HCl (116.76 mg, 1.68 mmol). The resulting mixture was stirred at 90° C. for 1 hours. After the reaction was completed, the mixture was diluted with EA (30 mL), washed with H₂O (20 mL). The organic layer was dried over anhydrous sodium sulfate, filtered, and evaporated to dryness. The reaction mixture was concentrated to give (S)-2-((4-((2-(4-chlorobenzyl)pyrimidin-4-yl)oxy)piperidin-1-yl)methyl)-N′-hydroxy-3-(oxetan-2-ylmethyl)-3H-imidazo[4,5-b]pyridine-6-carboximidamide (130 mg, crude) as a yellow solid. MS Calcd.: 562.2; MS Found: 563.1 [M+H]⁺.

### Step E: 3-(2-{[4-({2-[(4-chlorophenyl)methyl]pyrimidin-4-yl}oxy)piperidin-1-yl]methyl}-3-{[(2S)-oxetan-2-yl]methyl}-3H-imidazo[4,5-b]pyridin-6-yl)-4,5-dihydro-1,2,4-oxadiazol-5-one

To a solution of (S)-2-((4-((2-(4-chlorobenzyl)pyrimidin-4-yl)oxy)piperidin-1-yl)methyl)-N′-hydroxy-3-(oxetan-2-ylmethyl)-3H-imidazo[4,5-b]pyridine-6-carboximidamide (100 mg, 0.18 mmol) in THE (2 mL) was added TEA (54 mg, 0.54 mmol) and CDI (43.24 mg, 0.27 mmol). The resulting mixture was stirred at 70° C. for 3 hours. The solvent was removed in vacuo. The residue was purified by Prep-HPLC to give 3-(2-{[4-({2-[(4-chlorophenyl)methyl]pyrimidin-4-yl}oxy)piperidin-1-yl]methyl}-3-{[(2S)-oxetan-2-yl]methyl}-3H-imidazo[4,5-b]pyridin-6-yl)-4,5-dihydro-1,2,4-oxadiazol-5-one Compound 144a (29 mg, yield: 28%) as a white solid.

MS Calcd.: 588.2; MS Found: 589.1 [M+H]⁺.

¹H NMR (400 MHz, DMSO-d₆): δ 8.77 (d, J=1.6 Hz, 1H), 8.41 (d, J=6.0 Hz, 1H), 8.35 (d, J=2.0 Hz, 1H), 7.30-7.37 (m, 4H), 6.73 (d, J=5.6 Hz, 1H), 5.12-5.20 (m, 1H), 4.97-5.10 (m, 1H), 4.77-4.85 (m, 1H), 4.65-4.73 (m, 1H), 4.45-4.52 (m, 1H), 4.31-4.38 (m, 1H), 4.08 (s, 2H), 3.95 (s, 2H), 2.62-2.80 (m, 3H), 2.33-2.51 (m, 3H), 1.90-1.98 (m, 2H), 1.60-1.60 (m, 2H).

### Example 25: 2-[(4-chlorophenyl)methyl]-6-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyridine (Compound 143a)

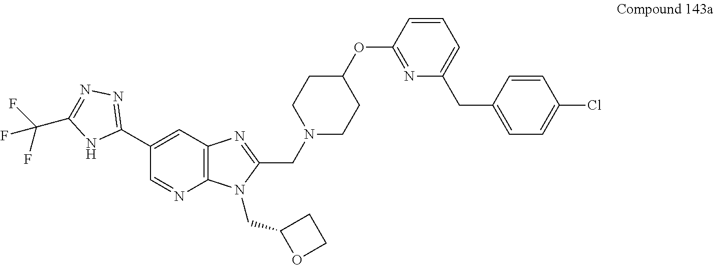 [SMILES: FC(F)(F)c1nnc(-c2cnc3c(c2)nc(CN2CCC(Oc4cccc(Cc5ccc(Cl)cc5)n4)CC2)n3C[C@@H]2CCO2)[nH]1]

Starting from 2-(4-chlorobenzyl)-6-(piperidin-4-yloxy)pyridine, 2-[(4-chlorophenyl)methyl]-6-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyridine Compound 143a (37 mg) as a white solid with the similar method of Compound 120a.

MS Calcd: 638.2; MS Found: 639.4 [M+H]⁺.

¹H NMR (400 MHz, DMSO-d₆): δ 8.99 (d, J=2.0 Hz, 1H), 8.56 (d, J=2.0 Hz, 1H), 7.59 (dd, J=8.4, 7.6 Hz, 1H), 7.29-7.38 (m, 4H), 6.82 (d, J=7.2 Hz, 1H), 6.58 (d, J=8.0 Hz, 1H), 5.15-5.21 (m, 1H), 4.92-5.00 (m, 1H), 4.81-4.86 (m, 1H), 4.68-4.75 (m, 1H), 4.47-4.53 (m, 1H), 4.34-4.40 (m, 1H), 3.90-4.00 (m, 4H), 2.72-2.83 (m, 2H), 2.65-2.72 (m, 1H), 2.45-2.54 (m, 1H), 2.34-2.43 (m, 2H), 1.90-1.97 (m, 2H), 1.55-1.70 (m, 2H). ¹⁹F-NMR (377 MHz): −62.99.

### Example 26: 2-[(4-chloro-2-fluorophenyl)methyl]-4-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyrimidine (Compound 142a)

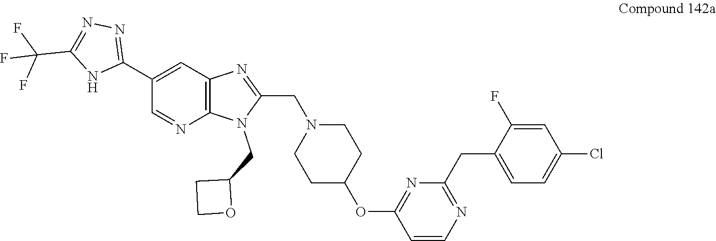 [SMILES: Fc1cc(Cl)ccc1Cc1nccc(OC2CCN(Cc3nc4cc(-c5nnc(C(F)(F)F)[nH]5)cnc4n3C[C@@H]3CCO3)CC2)n1]

Starting from 2-(4-chloro-2-fluorobenzyl)-4-(piperidin-4-yloxy)pyrimidine hydrochloride, 2-[(4-chloro-2-fluorophenyl)methyl]-4-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyrimidine Compound 142a (9 mg) as a white solid with the similar method of Compound 120a.

MS Calcd: 657.2; MS Found: 658.7 [M+H]⁺.

¹H NMR (400 MHz, DMSO-d₆): δ 15.41 (brs, 1H), 8.99 (d, J=1.6 Hz, 1H), 8.59 (d, J=1.6 Hz, 1H), 8.41 (d, J=6.0 Hz, 1H), 7.35-7.42 (m, 2H), 7.21-7.26 (m, 1H), 6.73 (d, J=5.6 Hz, 1H), 5.14-5.21 (m, 1H), 4.79-4.92 (m, 2H), 4.68-4.73 (m, 1H), 4.46-4.52 (m, 1H), 4.32-4.40 (m, 1H), 4.15 (s, 2H), 3.90-4.00 (m, 2H), 2.65-2.81 (m, 3H), 2.42-2.54 (m, 1H), 2.27-2.37 (m, 2H), 1.85-1.93 (m, 2H), 1.55-1.70 (m, 2H). ¹⁹F-NMR (377 MHz): −63.64, −114.06.

### Example 27: 2-[(4-chloro-2-fluorophenyl)methyl]-6-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyridine (Compound 141a)

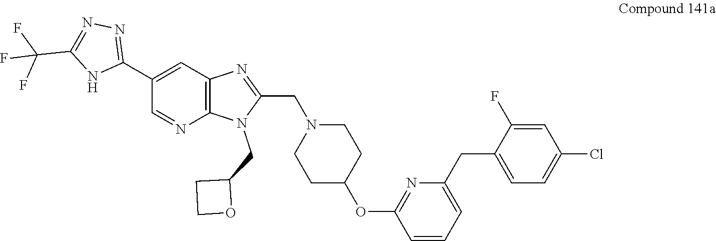 [SMILES: Fc1cc(Cl)ccc1Cc1cccc(OC2CCN(Cc3nc4cc(-c5nnc(C(F)(F)F)[nH]5)cnc4n3C[C@@H]3CCO3)CC2)n1]

Starting from 2-(4-chloro-2-fluorobenzyl)-6-(piperidin-4-yloxy)pyridine hydrochloride, 2-[(4-chloro-2-fluorophenyl)methyl]-6-({1-[(3-{[(2S)-oxetan-2-yl]methyl}-6-[5-(trifluoromethyl)-4H-1,2,4-triazol-3-yl]-3H-imidazo[4,5-b]pyridin-2-yl)methyl]piperidin-4-yl}oxy)pyridine Compound 141a (20 mg) as a white solid with the similar method of Compound 120a.

MS Calcd: 656.2; MS Found: 657.4 [M+H]⁺.

¹H NMR (400 MHz, DMSO-d₆): δ 15.38 (brs, 1H), 8.99 (d, J=1.6 Hz, 1H), 8.60 (d, J=2.0 Hz, 1H), 7.60 (t, J=8.4 Hz, 1H), 7.34-7.40 (m, 2H), 7.23 (dd, J=8.4 Hz, 2.0 Hz, 1H), 6.82 (d, J=7.2 Hz, 1H), 6.58 (d, J=8.0 Hz, 1H), 5.14-5.22 (m, 1H), 4.80-4.88 (m, 2H), 4.68-4.75 (m, 1H), 4.47-4.53 (m, 1H), 4.33-4.40 (m, 1H), 4.01 (s, 2H), 3.90-4.00 (m, 2H), 2.65-2.82 (m, 3H), 2.43-2.55 (m, 1H), 2.27-2.36 (m, 2H), 1.83-1.92 (m, 2H), 1.50-1.64 (m, 2H). ¹⁹F-NMR (377 MHz): −63.65, −114.57.

### Example 28: 2-{[4-({2-[(4-chlorophenyl)methyl]pyrimidin-4-yl}oxy)piperidin-1-yl]methyl}-5-(3-methyl-1H-pyrazol-5-yl)-1-{[(2S)-oxetan-2-yl]methyl}-1H-1,3-benzodiazole (Compound 131a)

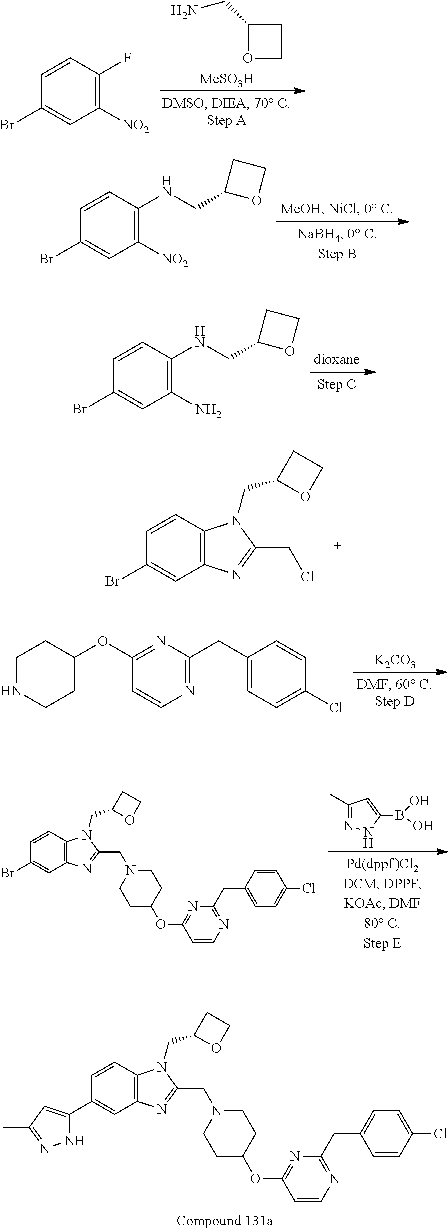

### Step A: (S)-4-bromo-2-nitro-N-(oxetan-2-ylmethyl)aniline

A mixture of 4-bromo-1-fluoro-2-nitrobenzene (10.0 g, 45.4 mmol), (S)-oxetan-2-ylmethanamine methanesulfonic acid (14.1 g, 77.0 mmol) and DIEA (17.5 g, 136.3 mmol) in DMSO (100 mL) was stirred at 70° C. for 5 hours. After the reaction was completed, the mixture was diluted with EA (50 mL×3), washed with H₂O (40 mL). The organic layer was dried over anhydrous sodium sulfate, filtered, and evaporated to dryness. The residue was purified by column chromatography on silica gel (PE/EA=5/1) to give (S)-4-bromo-2-nitro-N-(oxetan-2-ylmethyl)aniline (11.5 g, 98% yield) as a red solid. MS Calcd.: 286.0; MS Found: 287.0 [M+H]⁺.

### Step B: (S)-4-bromo-N1-(oxetan-2-ylmethyl)benzene-1,2-diamine

To a solution of (S)-4-bromo-2-nitro-N-(oxetan-2-ylmethyl)aniline (5.0 g, 17.5 mmol) in MeOH (100 mL) was added NiCl₂ (4.5 g, 34.9 mmol), The resulting mixture was stirred at 0° C. for 20 min, then added with NaBH₄ (2.0 g, 52.4 mmol). The resulting mixture was stirred at 0° C. for 30 min, quenched with water (200 mL), extracted with ethyl acetate (150 mL×3). The organic layers were combined, dried over Na₂SO₄. The residue was purified by column chromatography on silica gel (PE/EA=3/1) to give (S)-4-bromo-N1-(oxetan-2-ylmethyl)benzene-1,2-diamine (3.0 g, 67% yield) as a yellow solid. MS Calcd.: 256.0; MS Found: 257.0 [M+H].

### Step C: (S)-5-bromo-2-(chloromethyl)-1-(oxetan-2-ylmethyl)-1H-benzo[d]imidazole

To a solution of (S)-4-bromo-N1-(oxetan-2-ylmethyl)benzene-1,2-diamine (3.0 g, 11.7 mmol) and 2-chloroacetic anhydride (2.4 g, 14.1 mmol) in dioxane (30 mL) was stirred at rt for 3 hours and then at 60° C. for 16 hours. The reaction mixture was quenched with water (80 mL), extracted with ethyl acetate (70 mL×3). The organic layers were combined, dried over Na₂SO₄, filtered, and concentrated under vacuum. The residue was purified by column chromatography on silica gel (PE/EA=3/1) to give (S)-5-bromo-2-(chloromethyl)-1-(oxetan-2-ylmethyl)-1H-benzo[d]imidazole (2.8 g, 71% yield) as a yellow solid. MS Calcd.: 314.0; MS Found: 315.0 [M+H]⁺.

### Step D: (S)-5-bromo-2-((4-((2-(4-chlorobenzyl)pyrimidin-4-yl)oxy)piperidin-1-yl)methyl)-1-(oxetan-2-ylmethyl)-1H-benzo[d]imidazole

To a mixture of (S)-5-bromo-2-(chloromethyl)-1-(oxetan-2-ylmethyl)-1H-benzo[d]imidazole (500 mg, 1.6 mmol) in DMF (10 mL) was added K₂CO₃ (2.1 g, 15.9 mmol) and 2-(4-chlorobenzyl)-4-(piperidin-4-yloxy)pyrimidine (1.0 g, 3.3 mmol). The resulting mixture was heated to 60° C. for 5 hours. After cooled down to room temperature, the mixture was quenched with water (50 mL), extracted with ethyl acetate (70 mL). The organic layers were combined, dried over Na₂SO₄, filtered and concentrated under vacuum. The residue was purified by column chromatography on silica gel (PE/EA=1/1) to give (S)-5-bromo-2-((4-((2-(4-chlorobenzyl)pyrimidin-4-yl)oxy)piperidin-1-yl)methyl)-1-(oxetan-2-ylmethyl)-1H-benzo[d]imidazole (1.0 g, 95% yield) as a yellow oil. MS Calcd.: 581.2; MS Found: 582.1M+H]⁺.

### Step E: 2-{[4-({2-[(4-chlorophenyl)methyl]pyrimidin-4-yl}oxy)piperidin-1-yl]methyl}-5-(3-methyl-1H-pyrazol-5-yl)-1-{[(2S)-oxetan-2-yl]methyl}-1H-1,3-benzodiazole

A mixture of (S)-5-bromo-2-((4-((2-(4-chlorobenzyl)pyrimidin-4-yl)oxy)piperidin-1-yl)methyl)-1-(oxetan-2-ylmethyl)-1H-benzo[d]imidazole (50 mg, 0.085 mmol), Pd(dppf)Cl₂·DCM (7 mg, 0.008 mmol), KOAc (25 mg, 0.26 mmol), DPPF (1.3 mg, 0.004 mmol), (3-methyl-1H-pyrazol-5-yl)boronic acid (21 mg, 0.17 mmol) in DMF (2 mL) was stirred at 80° C. for 4 hours under N₂. After the reaction was completed, the reaction mixture was purified directly by Prep-HPLC to give 2-{[4-({2-[(4-chlorophenyl)methyl]pyrimidin-4-yl}oxy)piperidin-1-yl]methyl}-5-(3-methyl-1H-pyrazol-5-yl)-1-{[(2S)-oxetan-2-yl]methyl}-1H-1,3-benzodiazole Compound 131a (3.0 mg, 5% yield) as a white solid.

MS Calcd.: 583.2; MS Found: 584.4 [M+H]⁺.

¹H NMR (400 MHz, DMSO-d₆): δ 8.41 (d, J=5.6 Hz, 1H), 7.92 (s, 1H), 7.59-7.65 (m, 2H), 7.30-7.38 (m, 4H), 6.73 (d, J=6.0 Hz, 1H), 6.43 (s, 1H), 4.98-5.12 (m, 2H), 4.66-4.72 (m, 1H), 4.53-4.60 (m, 1H), 4.46-4.53 (m, 1H), 4.35-4.41 (m, 1H), 4.08 (s, 2H), 3.90 (d, J=13.6 Hz, 1H), 3.76 (d, J=13.2 Hz, 1H), 2.65-2.80 (m, 3H), 2.40-2.50 (m, 1H), 2.30-2.40 (m, 2H), 2.25 (s, 3H), 1.88-1.97 (m, 2H), 1.55-1.69 (m, 2H).

### Example 29: 2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazole-5-carboxylic acid (Compound 134a)

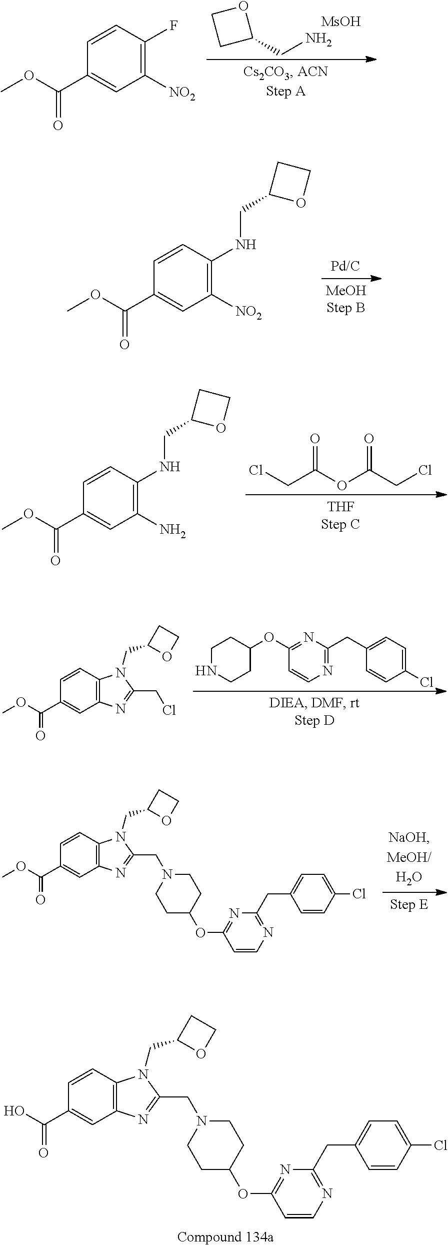

### Step A: Methyl (S)-3-nitro-4-((oxetan-2-ylmethyl)amino)benzoate

To a solution of methyl 4-fluoro-3-nitrobenzoate (500 mg, 2.5 mmol) and (S)-oxetan-2-ylmethanamine MsOH salt (460 mg, 2.5 mmol) in ACN (10 mL) was added Cs₂CO₃ (2.45 g, 7.5 mmol) at room temperature. The reaction was stirred at 70° C. for 3 hours. After the reaction was completed, the reaction was quenched with water (50 mL) and extracted with ethyl acetate (100 mL×3). The organic layers were combined and washed with brine (100 mL×2), dried over sodium sulfate, filtered, and concentrated in vacuum. The residue was purified by column chromatography to give methyl (S)-3-nitro-4-((oxetan-2-ylmethyl) amino) benzoate (500 mg, 72% yield) as an orange solid. MS Calcd.: 266.1; MS Found: 267.3 [M+H]⁺.

### Step B: Methyl (S)-3-amino-4-((oxetan-2-ylmethyl)amino)benzoate

To a solution of methyl (S)-3-nitro-4-((oxetan-2-ylmethyl) amino) benzoate (100 mg, 0.14 mmol) in MeOH (2 mL) was added wet Pd/C (20 mg, 50 wt %). The reaction vessel was purged with H₂ for three times and the reaction was stirred at room temperature under H₂ (15 psi) for 1 hour. After the reaction was completed, the reaction was filtered and concentrated under reduced pressure to afford methyl (S)-3-amino-4-((oxetan-2-ylmethyl)amino)benzoate (100 mg, crude) as colorless oil. MS Calcd.: 236.1; MS Found: 237.0 [M+H]⁺.

### Step C: Methyl (S)-2-(chloromethyl)-1-(oxetan-2-ylmethyl)-1H-benzo[d]imidazole-5-carboxylate

To a solution of methyl (S)-3-amino-4-((oxetan-2-ylmethyl) amino) benzoate (100 mg, crude) in THE (3 mL) was added 2-chloroacetic anhydride (79.7 mg, 0.47 mmol). The reaction was stirred at room temperature for 1 hours, then stirred at 80° C. for 72 hours. After the reaction was completed, the reaction was concentrated and purified by column chromatography to give methyl (S)-2-(chloromethyl)-1-(oxetan-2-ylmethyl)-1H-benzo[d]imidazole-5-carboxylate (50 mg, 40% yield in two steps) as a grey solid. MS Calcd.: 294.1; MS Found: 295.1 [M+H]⁺.

### Step D: methyl (S)-2-((4-((2-(4-chlorobenzyl)pyrimidin-4-yl)oxy)piperidin-1-yl)methyl)-1-(oxetan-2-ylmethyl)-1H-benzo[d]imidazole-5-carboxylate

To a solution of 2-(4-chlorobenzyl)-4-(piperidin-4-yloxy)pyrimidine (409 mg) in DMF (2 mL) was added DIEA (133 mg, 1.02 mmol) and methyl (S)-2-(chloromethyl)-1-(oxetan-2-ylmethyl)-1H-benzo[d]imidazole-5-carboxylate (100 mg, 0.340 mmol) at room temperature. The reaction was stirred at room temperature for 2 hours, quenched with water (10 mL) and extracted with ethyl acetate (20 mL×3). The organic layer was combined and washed with brine (10 mL×5), dried over sodium sulfate, filtered and concentrated in vacuum. The residue was purified by column chromatography (DCM/MeOH=10/1) to give the methyl (S)-2-((4-((2-(4-chlorobenzyl)pyrimidin-4-yl)oxy)piperidin-1-yl)methyl)-1-(oxetan-2-ylmethyl)-1H-benzo[d]imidazole-5-carboxylate (80 mg, 42.1% yield) as yellow oil. MS Calcd.: 561.2. MS Found: 562.2[M+H]⁺.

### Step E: 2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazole-5-carboxylic acid (Compound 134a)

To a solution of methyl (S)-2-((4-((2-(4-chlorobenzyl)pyrimidin-4-yl)oxy)piperidin-1-yl)methyl)-1-(oxetan-2-ylmethyl)-1H-benzo[d]imidazole-5-carboxylate (60 mg, 0.107 mmol) in MeOH (1 mL) and H₂O (0.1 mL) was added NaOH (5 mg, 0.125 mmol) at room temperature. The reaction was stirred at room temperature for 2 hours. After the reaction was completed, the reaction mixture was purified directly by Prep-HPLC to give 2-{[4-({2-[(4-chlorophenyl)methyl]pyrimidin-4-yl}oxy)piperidin-1-yl]methyl}-1-{[(2S)-oxetan-2-yl]methyl}-1H-1,3-benzodiazole-5-carboxylic acid Compound 134a (6.54 mg, 11.2% yield) as a white solid.

MS Calcd.: 547.2; MS Found: 548.2 [M+H]⁺.

¹H NMR (400 MHz, DMSO-d₆): δ 8.40 (d, J=6.0 Hz, 1H), 8.04 (s, 1H), 7.77 (dd, J=8.4 Hz, J=1.6 Hz, 1H), 7.40 (d, J=8.40 Hz, 1H), 7.31-7.38 (m, 4H), 6.73 (d, J=6.0 Hz, 1H), 4.95-5.13 (m, 2H), 4.68-4.73 (m. 1H), 4.45-4.60 (m, 2H), 4.34-4.40 (m, 1H), 4.08 (s, 2H), 3.89 (d, J=13.2 Hz, 1H), 3.75 (d, J=13.6 Hz, 1H), 2.64-2.80 (m. 3H), 2.38-2.53 (m. 1H), 2.28-2.38 (m, 2H), 1.87-1.97 (m, 2H), 1.55-1.70 (m, 2H).

### Example 30: 2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazole-5-carboxylic acid (Compound 135a)

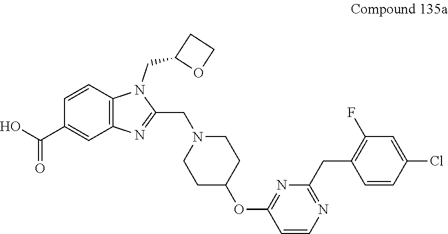 [SMILES: O=C(O)c1ccc2c(c1)nc(CN1CCC(Oc3ccnc(Cc4ccc(Cl)cc4F)n3)CC1)n2C[C@@H]1CCO1]

2-{[4-({2-[(4-chloro-2-fluorophenyl)methyl]pyrimidin-4-yl}oxy)piperidin-1-yl]methyl}-1-{[(2S)-oxetan-2-yl]methyl}-1H-1,3-benzodiazole-5-carboxylic acid Compound 135a (83 mg) was obtained with the similar method of Compound 134a as white solid.

MS Calcd.: 565.2; MS Found: 566.1 [M+H]⁺.

¹H NMR (400 MHz, DMSO-d₆): δ 8.48 (d, J=6.0 Hz, 1H), 8.39 (d, J=0.8 Hz, 1H), 7.91 (dd, J=8.4 Hz, J=1.6 Hz, 1H), 7.79-7.83 (m, 1H), 7.37-7.45 (m, 2H), 7.25 (dd, J=8.0 Hz, J=1.6 Hz, 1H), 6.81 (d, J=6.0 Hz, 1H), 5.12-5.23 (m, 1H), 5.00-5.07 (m, 1H), 4.78-4.94 (m, 3H), 4.64-4.71 (m, 1H), 4.46-4.53 (m, 1H), 4.30-4.38 (m, 1H), 4.17 (s, 2H), 3.35-3.65 (m, 4H), 2.66-2.78 (m, 1H), 2.27-2.38 (m, 1H), 2.13-2.24 (m, 2H), 1.95-2.10 (m, 2H). ¹⁹F-NMR (377 MHz): −114.01.

### Example 31: 2-{[4-({6-[(4-chloro-2-fluorophenyl)methyl]pyridin-2-yl}oxy)piperidin-1-yl]methyl}-1-{[(2S)-oxetan-2-yl]methyl}-5-[5-(trifluoromethyl)-4H-1,2,4-triazol-3-yl]-1H-1,3-benzodiazole (Compound 137a)

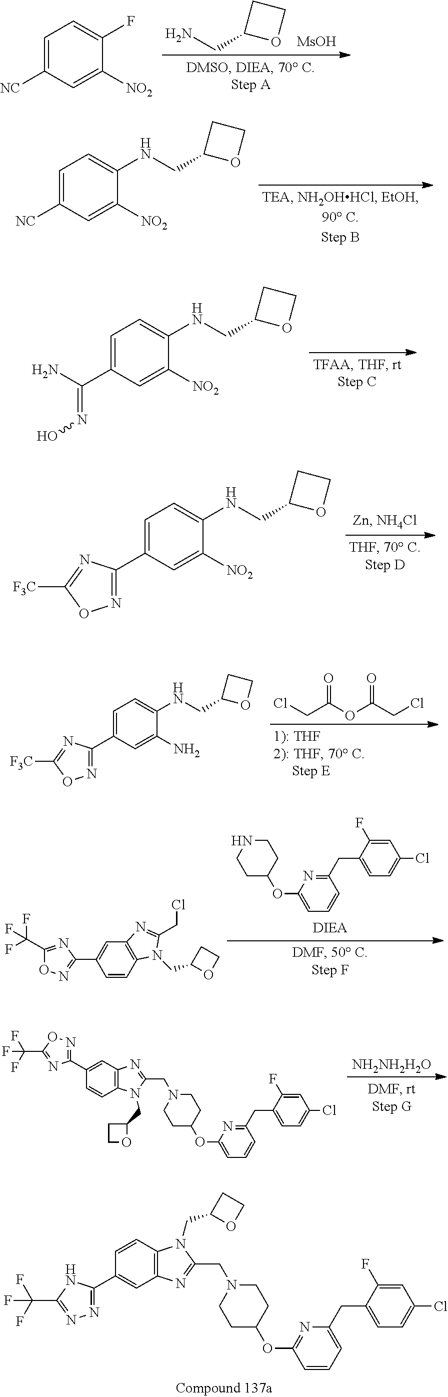

### Step A: (S)-3-nitro-4-((oxetan-2-ylmethyl)amino)benzonitrile

To a solution of 4-fluoro-3-nitrobenzonitrile (5 g, 30.1 mmol) and (S)-oxetan-2-ylmethanamine MsOH salt (6.1 g, 33.1 mmol) in DMSO (60 mL) was added DIEA (11.6 g, 90.3 mmol) at room temperature. The reaction was stirred at 70° C. for 1 hour. After the reaction was completed, the reaction was quenched with water (50 mL) and extracted with ethyl acetate (200 mL×3). The organic layer was combined and washed with brine (100 mL×2), dried over sodium sulfate, filtered, and concentrated in vacuum. The residue was purified by column chromatography (PE:EA=3:1) to give (S)-3-nitro-4-((oxetan-2-ylmethyl)amino)benzonitrile (4.9 g, 700%) as an orange solid. MS Calcd.: 233.1; MS Found: 234.1 [M+H]⁺.

### Step B: (S′)—N′-hydroxy-3-nitro-4-((oxetan-2-ylmethyl)amino)benzimidamide

To a solution of (S)-3-nitro-4-((oxetan-2-ylmethyl)amino)benzonitrile (1.0 g, 4.3 mmol) in EtOH (10 mL) was added Hydroxylamine hydrochloride (1.8 g, 2.8 mmol) and TEA (1.73 g, 17.1 mmol). The reaction was stirred at 90° C. for 16 hours. After the reaction was completed, the reaction was quenched with water (50 mL) and extracted with ethyl acetate (100 mL×3). The organic layer was combined and washed with brine (50 mL×2), dried over sodium sulfate, filtered, and concentrated in vacuum. The residue was purified by column chromatography (DCM:MeOH=10:1) to give (S)—N′-hydroxy-3-nitro-4-((oxetan-2-ylmethyl)amino)benzimidamide (840 mg, 73% yield) as red solid. MS Calcd.: 266.1; MS Found: 267.1 [M+H]⁺.

### Step C: (S)-2-nitro-N-(oxetan-2-ylmethyl)-4-(5-(trifluoromethyl)-1,2,4-oxadiazol-3-yl)aniline

To a solution of (S)—N′-hydroxy-3-nitro-4-((oxetan-2-ylmethyl)amino)benzimidamide (300 mg, 1.13 mmol) in THE (5 mL) was added TFAA (945 g, 4.5 mmol) at room temperature. The reaction was stirred at room temperature for 2 hours. After the reaction was completed, the reaction was quenched with NaHCO₃ solution (50 mL) and extracted with ethyl acetate (50 mL×3). The organic layer was combined and washed with brine (50 mL×2), dried over sodium sulfate, filtered and concentrated in vacuum. The residue was purified by column chromatography (PE:EA=5:1) to give (S)-2-nitro-N-(oxetan-2-ylmethyl)-4-(5-(trifluoromethyl)-1,2,4-oxadiazol-3-yl)aniline (300 mg, 77% yield) as yellow solid. MS Calcd.: 344.1; MS Found: 345.1 [M+H]⁺.

### Step D: (S)—N1-(oxetan-2-ylmethyl)-4-(5-(trifluoromethyl)-1,2,4-oxadiazol-3-yl)benzene-1,2-diamine

To a solution of (S)-2-nitro-N-(oxetan-2-ylmethyl)-4-(5-(trifluoromethyl)-1,2,4-oxadiazol-3-yl)aniline (300 mg, 0.87 mmol) in THF/H₂O (10 mL/1 ml) was added Zn (1.8 g, 27.5 mmol) and NH₄Cl (2.9 g, 55.0 mmol) at room temperature. The reaction was stirred at 70° C. for 2 hours. After the reaction was completed, the reaction was filtered and concentrated in vacuum. The residue was purified by column chromatography (PE:EA=1:1) to give (S)—N1-(oxetan-2-ylmethyl)-4-(5-(trifluoromethyl)-1,2,4-oxadiazol-3-yl)benzene-1,2-diamine (230 mg, 85% yield) as yellow solid. MS Calcd.: 314.1; MS Found: 315.1 [M+H]⁺.

### Step E: (S)-3-(2-(chloromethyl)-1-(oxetan-2-ylmethyl)-1H-benzo[d]imidazol-5-yl)-5-(trifluoromethyl)-1,2,4-oxadiazole

To a solution of (S)—N1-(oxetan-2-ylmethyl)-4-(5-(trifluoromethyl)-1,2,4-oxadiazol-3-yl)benzene-1,2-diamine (230 mg, 0.73 mmol) in THF (6 mL) was added 2-chloroacetic anhydride (137 mg, 0.80 mmol) at room temperature. The reaction was stirred at room temperature for 2 hours. Then the reaction was stirred at 70° C. for 3 days. After the reaction was completed, the reaction was concentrated in vacuum. The residue was purified by column chromatography (PE:EA=2:1) to give (S)-3-(2-(chloromethyl)-1-(oxetan-2-ylmethyl)-1H-benzo[d]imidazol-5-yl)-5-(trifluoromethyl)-1,2,4-oxadiazole (120 mg, 34% yield) as yellow solid. MS Calcd.: 372.1; MS Found: 373.1 [M+H]⁺.

### Step F: (S)-3-(2-((4-((6-(4-chloro-2-fluorobenzyl)pyridin-2-yl)oxy)piperidin-1-yl)methyl)-1-(oxetan-2-ylmethyl)-1H-benzo[d]imidazol-5-yl)-5-(trifluoromethyl)-1,2,4-oxadiazole

A mixture of 2-(4-chloro-2-fluorobenzyl)-6-(piperidin-4-yloxy)pyridine (43 mg) and DIEA (50 mg, 0.39 mmol) in DMF (1 mL) was stirred at room temperature for 10 minutes. (S)-3-(2-(chloromethyl)-1-(oxetan-2-ylmethyl)-1H-benzo[d]imidazol-5-yl)-5-(trifluoromethyl)-1,2,4-oxadiazole (50 mg, 0.13 mmol) was added and the mixture was stirred at 50° C. for 3 hours. After the reaction was completed, the mixture was concentrated in vacuum. The crude product was purified by column chromatography on silica gel (PE:EA=1:1) to afford (S)-3-(2-((4-((6-(4-chloro-2-fluorobenzyl)pyridin-2-yl)oxy)piperidin-1-yl)methyl)-1-(oxetan-2-ylmethyl)-1H-benzo[d]imidazol-5-yl)-5-(trifluoromethyl)-1,2,4-oxadiazole (70 mg, Yield: 84%) as white solid. MS Calcd: 656.2; MS Found: 657.2 [M+H]⁺.

### Step G: 2-{[4-({6-[(4-chloro-2-fluorophenyl)methyl]pyridin-2-yl}oxy)piperidin-1-yl]methyl}-1-{[(2S)-oxetan-2-yl]methyl}-5-[5-(trifluoromethyl)-4H-1,2,4-triazol-3-yl]-1H-1,3-benzodiazole

To a mixture of (S)-3-(2-((4-((6-(4-chloro-2-fluorobenzyl)pyridin-2-yl)oxy)piperidin-1-yl)methyl)-1-(oxetan-2-ylmethyl)-1H-benzo[d]imidazol-5-yl)-5-(trifluoromethyl)-1,2,4-oxadiazole (70 mg, 0.11 mmol) in DMF (2 mL) was added NH₂NH₂·H₂O (16 mg, 0.33 mmol), the mixture was stirred at room temperature for 2 hours. After the reaction was completed, the reaction mixture was purified directly by Prep-HPLC to give 2-{[4-({6-[(4-chloro-2-fluorophenyl)methyl]pyridin-2-yl}oxy)piperidin-1-yl]methyl}-1-{[(2S)-oxetan-2-yl]methyl}-5-[5-(trifluoromethyl)-4H-1,2,4-triazol-3-yl]-1H-1,3-benzodiazole (Compound 137a) (5 mg, yield: 7%) as a white solid. MS Calcd: 655.21; MS Found: 656.4 [M+H]⁺.

¹H NMR (400 MHz, DMSO-d₆): δ 8.24 (s, 1H), 7.92 (dd, J=8.4 Hz, 1.6 Hz, 1H), 7.77 (d, J=8.8 Hz, 1H), 7.60 (t, J=8.0 Hz, 1H), 7.35-7.40 (m, 2H), 7.24 (dd, J=8.4 Hz, 2.0 Hz, 1H), 6.82 (d, J=7.6 Hz, 1H), 6.58 (d, J=8.0 Hz, 1H), 5.07-5.14 (m, 1H), 4.71-4.86 (m, 2H), 4.61 (dd, J=15.2 Hz, 2.8 Hz, 1H), 4.47-4.53 (m, 1H), 4.37-4.42 (m, 1H), 4.01 (s, 2H), 3.93 (d, J=13.6 Hz, 1H), 3.78 (d, J=13.6 Hz, 1H), 2.78-2.83 (m, 1H), 2.65-2.78 (m, 2H), 2.35-2.51 (m, 1H), 2.20-2.35 (m, 2H), 1.80-1.91 (m, 2H), 1.48-1.60 (m, 2H). ¹⁹F-NMR (377 MHz): −63.05, −63.10, −63.11, −114.58.

### Example 32: 2-{[4-({6-[(4-chlorophenyl)methyl]pyridin-2-yl}oxy)piperidin-1-yl]methyl}-1-{[(2S)-oxetan-2-yl]methyl}-5-[5-(trifluoromethyl)-4H-1,2,4-triazol-3-yl]-1H-1,3-benzodiazole (Compound 136a)

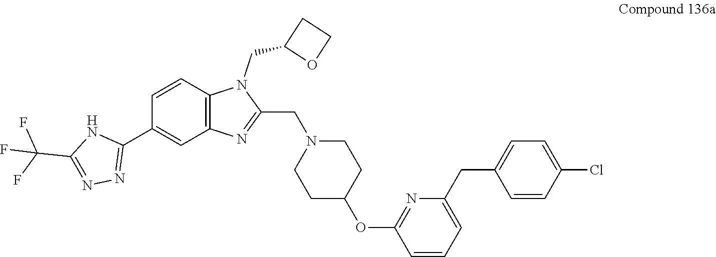 [SMILES: FC(F)(F)c1nnc(-c2ccc3c(c2)nc(CN2CCC(Oc4cccc(Cc5ccc(Cl)cc5)n4)CC2)n3C[C@@H]2CCO2)[nH]1]

2-{[4-({6-[(4-chlorophenyl)methyl]pyridin-2-yl}oxy)piperidin-1-yl]methyl}-1-{[(2S)-oxetan-2-yl]methyl}-5-[5-(trifluoromethyl)-4H-1,2,4-triazol-3-yl]-1H-1,3-benzodiazole Compound 136a (17 mg) was obtained with the similar method of Compound 137a as a white solid.

MS Calcd: 637.2; MS Found: 638.4 [M+H]⁺.

¹H NMR (400 MHz, DMSO-d₆): δ 15.05 (brs, 1H), 8.26 (s, 1H), 7.92 (dd, J=8.4 Hz, 1.2 Hz, 1H), 7.81 (d, J=8.4 Hz, 1H), 7.59 (t, J=7.6 Hz, 1H), 7.28-7.37 (m, 4H), 6.82 (d, J=7.2 Hz, 1H), 6.58 (d, J=8.0 Hz, 1H), 5.08-5.15 (m, 1H), 4.90-5.00 (m, 1H), 4.77 (dd, J=15.2 Hz, 7.2 Hz, 1H), 4.59-4.66 (m, 1H), 4.47-4.54 (m, 1H), 4.37-4.44 (m, 1H), 3.90-3.95 (m, 3H), 3.80 (d, J=13.6 Hz, 1H), 2.65-2.85 (m, 3H), 2.40-2.51 (m, 1H), 2.30-2.40 (m, 2H), 1.86-1.97 (m, 2H), 1.53-1.66 (m, 2H). ¹⁹F-NMR (377 MHz): −63.58.

### Example 33: 2-{[4-({2-[(4-chlorophenyl)methyl]pyrimidin-4-yl}oxy)piperidin-1-yl]methyl}-1-{[(3R)-oxolan-3-yl]methyl}-5-[5-(trifluoromethyl)-4H-1,2,4-triazol-3-yl]-1H-1,3-benzodiazole (Compound 148a)

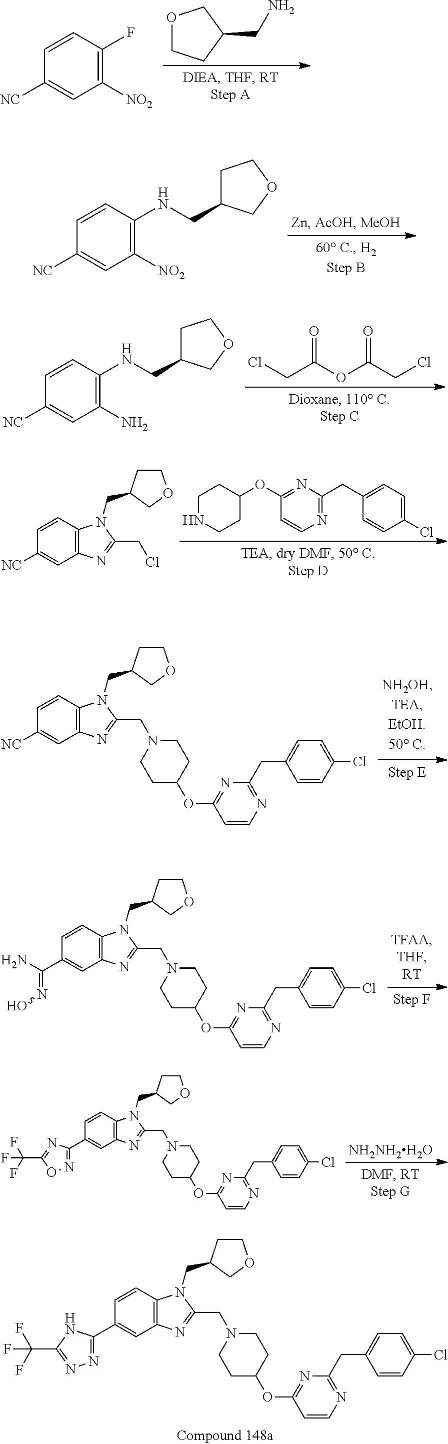

### Step A: (R)-3-nitro-4-(((tetrahydrofuran-3-yl)methyl)amino)benzonitrile

A mixture of 4-fluoro-3-nitrobenzonitrile (300 mg, 1.8 mmol), (R)-(tetrahydrofuran-3-yl)methanamine (182 mg, 1.8 mmol), N,N-Diisopropylethylamine (708 mg, 5.4 mmol) in THF (10 mL) was stirred at room temperature for 4 hrs. After the reaction was completed, the mixture was diluted with ethyl acetate (30 mL), washed with brine (20 mL×2). The organic layer was dried over sodium sulfate, filtered, and concentrated in vacuum. The residue was purified by column chromatography on silica (PE/EA=4/1) gel to give (R)-3-nitro-4-(((tetrahydrofuran-3-yl)methyl)amino)benzonitrile (440 mg, yield: 98%). MS Calcd.: 247.1; MS Found: 248.1 [M+H]⁺.

### Step B: (R)-3-amino-4-(((tetrahydrofuran-3-yl)methyl)amino)benzonitrile

A mixture of (R)-3-nitro-4-(((tetrahydrofuran-3-yl)methyl)amino)benzonitrile (400 mg, 1.3 mmol), Zinc powder (1.1 g, 16.2 mmol), acetic acid (292 mg, 4.9 mmol) in MeOH (15 mL) was stirred at 60° C. for 6 hours under H₂. After the reaction was completed, the mixture was filtered. The filtrate was diluted with EA (30 mL), then washed with Sat. NaHCO₃ (25 mL) and water (30 mL×2) successively. The organic layer was dried over sodium sulfate, filtered, and concentrated in vacuum. The residue was purified by column chromatography on silica gel (PE/EA=4/1) to give (R)-3-amino-4-(((tetrahydrofuran-3-yl)methyl)amino)benzonitrile (340 mg, yield: 96%). MS Calcd.: 217.1; MS Found: 218.1 [M+H]⁺.

### Step C: (R)-2-(chloromethyl)-1-((tetrahydrofuran-3-yl)methyl)-1H-benzo[d]imidazole-5-carbonitrile

A mixture of (R)-3-amino-4-(((tetrahydrofuran-3-yl)methyl)amino)benzonitrile (260 mg, 1.2 mmol), 2-chloroacetic anhydride (244.0 mg, 1.4 mmol) in dioxane (5 mL) was stirred at 110° C. for 8 h. After the reaction was completed, the mixture was diluted with EA (25 mL), then washed with water (25 mL×2). The organic layer was dried over sodium sulfate, filtered, and concentrated in vacuum. The residue was purified by column chromatography on silica gel (PE/EA=3/1) to give (R)-2-(chloromethyl)-1-((tetrahydrofuran-3-yl)methyl)-1H-benzo[d]imidazole-5-carbonitrile (300 mg, yield: 90%). MS Calcd.: 275.1; MS Found: 276.0 [M+H]⁺.

### Step D: (R)-2-((4-((2-(4-chlorobenzyl)pyrimidin-4-yl)oxy)piperidin-1-yl)methyl)-1-((tetrahydrofuran-3-yl)methyl)-1H-benzo[d]imidazole-5-carbonitrile

A mixture of (R)-2-(chloromethyl)-1-((tetrahydrofuran-3-yl)methyl)-1H-benzo[d]imidazole-5-carbonitrile (104 mg, 0.38 mmol), 2-(4-chlorobenzyl)-4-(piperidin-4-yloxy)pyrimidine (115 mg, 0.38 mmol) and Et₃N (230 mg, 2.28 mmol) in dry DMF (6 mL) was stirred at 50° C. for 7 h. After the reaction was completed, the mixture was diluted with EA (15 mL), then washed with water (15 mL×2). The organic layer was dried over sodium sulfate, filtered and concentrated in vacuum. The residue was purified by Prep-TLC (DCM/MeOH=10/1) to give (R)-2-((4-((2-(4-chlorobenzyl)pyrimidin-4-yl)oxy)piperidin-1-yl)methyl)-1-((tetrahydrofuran-3-yl)methyl)-1H-benzo[d]imidazole-5-carbonitrile (56 mg, yield: 27%). MS Calcd.: 542.2; MS Found: 543.2 [M+H]⁺.

### Step E: (R)-2-((4-((2-(4-chlorobenzyl)pyrimidin-4-yl)oxy)piperidin-1-yl)methyl)-N′-hydroxy-1-((tetrahydrofuran-3-yl)methyl)-1H-benzo[d]imidazole-5-carboximidamide

A mixture of (R)-2-((4-((2-(4-chlorobenzyl)pyrimidin-4-yl)oxy)piperidin-1-yl)methyl)-1-((tetrahydrofuran-3-yl)methyl)-1H-benzo[d]imidazole-5-carbonitrile (50 mg, 0.092 mmol), NH₂OH (50% aq., 18.3 mg, 0.55 mmol) and Et₃N (55 mg, 0.54 mmol) in EtOH (3 mL) was stirred at 50° C. for 3 h. After the reaction was completed, the mixture was diluted with EA (10 mL), then washed with brine (10 mL). The organic layer was dried over sodium sulfate, filtered and concentrated in vacuum to afford (R)-2-((4-((2-(4-chlorobenzyl)pyrimidin-4-yl)oxy)piperidin-1-yl)methyl)-N′-hydroxy-1-((tetrahydrofuran-3-yl)methyl)-1H-benzo[d]imidazole-5-carboximidamide (65 mg, crude, yield: 100%). MS Calcd.: 575.2; MS Found: 576.3 [M+H]⁺.

### Step F: (R)-3-(2-(4-(2-(4-chlorobenzyl)pyrimidin-4-yl)oxy)piperidin-1-yl)methyl)-1-((tetrahydrofuran-3-yl)methyl)-1H-benzo[d]imidazol-5-yl)-5-(trifluoromethyl)-1,2,4-oxadiazole

A mixture of (R)-2-((4-((2-(4-chlorobenzyl)pyrimidin-4-yl)oxy)piperidin-1-yl)methyl)-N′-hydroxy-1-((tetrahydrofuran-3-yl)methyl)-1H-benzo[d]imidazole-5-carboximidamide (65.0 mg), Trifluoroacetic anhydride (60.0 mg, 0.29 mmol) in THE (4 mL) was stirred at room temperature for 2 h. After the reaction was completed, the mixture was diluted with EA (10 mL) and washed with Sat. NaHCO₃ (5 mL). The organic layer was dried over sodium sulfate, filtered and concentrated in vacuum. The residue was purified by Prep-TLC (DCM/MeOH=10/1) to give (R)-3-(2-((4-((2-(4-chlorobenzyl)pyrimidin-4-yl)oxy)piperidin-1-yl)methyl)-1-((tetrahydrofuran-3-yl)methyl)-1H-benzo[d]imidazol-5-yl)-5-(trifluoromethyl)-1,2,4-oxadiazole (39 mg, yield: 62%). MS Calcd.: 653.2; MS Found: 654.2 [M+H]⁺.

### Step G: 2-{[4-({2-[(4-chlorophenyl)methyl]pyrimidin-4-yl}oxy)piperidin-1-yl]methyl}-1-{[(3R)-oxolan-3-yl]methyl}-5-[5-(trifluoromethyl)-4H-1,2,4-triazol-3-yl]-1H-1,3-benzodiazole (Compound 148a)

A mixture of (R)-3-(2-((4-((2-(4-chlorobenzyl)pyrimidin-4-yl)oxy)piperidin-1-yl)methyl)-1-((tetrahydrofuran-3-yl)methyl)-1H-benzo[d]imidazol-5-yl)-5-(trifluoromethyl)-1,2,4-oxadiazole (39 mg, 0.06 mmol), hydrazine hydrate (17 mg, 0.36 mmol) in DMF (2 mL) was stirred at room temperature for 2 h. After the reaction was completed, the reaction mixture was purified directly by prep-HPLC to give 2-{[4-({2-[(4-chlorophenyl)methyl]pyrimidin-4-yl}oxy)piperidin-1-yl]methyl}-1-{[(3R)-oxolan-3-yl]methyl}-5-[5-(trifluoromethyl)-4H-1,2,4-triazol-3-yl]-1H-1,3-benzodiazole Compound 148a (1 mg, yield: 2%) as white solid.

MS Calcd.: 652.2; MS Found: 653.2 [M+H]⁺.

¹H NMR (400 MHz, CD₃OD): δ 8.34 (d, J=6.0 Hz, 1H), 8.30 (d, J=0.8 Hz, 1H), 8.00 (dd, J=8.4 Hz, 1.6 Hz, 1H), 7.76 (d, J=8.4 Hz, 1H), 7.25-7.32 (m, 4H), 6.66 (d, J=5.6 Hz, 1H), 5.07-5.16 (m, 1H), 4.45 (d, J=7.6 Hz, 2H), 4.11 (s, 2H), 4.00-4.09 (m, 1H), 3.85-3.94 (m, 2H), 3.77-3.83 (m, 1H), 3.62-3.72 (m, 2H), 3.05-3.15 (m, 1H), 2.77-2.86 (m, 2H), 2.40-2.50 (m, 2H), 1.93-2.12 (m, 3H), 1.70-1.90 (m, 3H). ¹⁹F-NMR (377 MHz): −66.53.

### Example 34: 2-{[4-({2-[(4-chlorophenyl)methyl]-5-fluoropyrimidin-4-yl}oxy)piperidin-1-yl]methyl}-1-{[(3R)-oxolan-3-yl]methyl}-5-[5-(trifluoromethyl)-4H-1,2,4-triazol-3-yl]-1H-1,3-benzodiazole (Compound 147a)

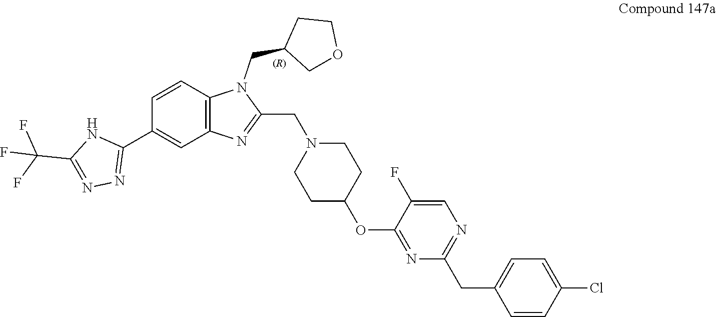

MS Calcd.: 670.2; MS Found: 671.2 [M+H]⁺.

¹H NMR (400 MHz, CD₃OD): δ 8.30-3.35 (m, 2H), 8.01 (dd, J=8.4 Hz, 1.6 Hz, 1H), 7.81 (d, J=8.4 Hz, 1H), 7.25-7.32 (m, 4H), 5.20-5.32 (m, 1H), 4.43 (d, J=8 Hz, 2H), 4.18-4.22 (m, 2H), 4.11 (s, 2H), 4.00-4.09 (m, 1H), 3.77-3.85 (m, 1H), 3.67-3.74 (m, 1H), 3.60-3.67 (m, 1H), 3.00-3.18 (m, 3H), 2.70-2.90 (m, 2H), 2.00-2.17 (m. 3H), 1.87-2.01 (s, 2H), 1.78-1.87 (m, 1H)¹⁹F-NMR (377 MHz): −66.78, −158.71.

### Example 35: 2-{[4-({2-[(4-chlorophenyl)methyl]pyrimidin-4-yl}oxy)piperidin-1-yl]methyl}-1-[(1H-pyrazol-4-yl)methyl]-5-(1H-1,2,3,4-tetrazol-5-yl)-1H-1,3-benzodiazole (Compound 153)

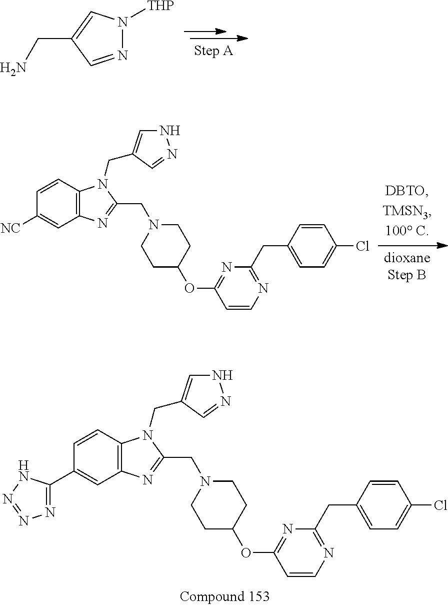

### Step A: 1-((1H-pyrazol-4-yl)methyl)-2-((4-((2-(4-chlorobenzyl)pyrimidin-4-yl)oxy)piperidin-1-yl)methyl)-1H-benzo[d]imidazole-5-carbonitrile

Starting from (1-(tetrahydro-2H-pyran-2-yl)-1H-pyrazol-4-yl)methanamine, 1-((1H-pyrazol-4-yl)methyl)-2-((4-((2-(4-chlorobenzyl)pyrimidin-4-yl)oxy)piperidin-1-yl)methyl)-1H-benzo[d]imidazole-5-carbonitrile (120 mg) was obtained with the similar method of Compound 106a as a brown solid. MS Calcd.: 538.2; MS Found: 539.2 [M+H]⁺.

### Step B: 2-{[4-({2-[(4-chlorophenyl)methyl]pyrimidin-4-yl}oxy)piperidin-1-yl]methyl}-1-[(1H-pyrazol-4-yl)methyl]-5-(1H-1,2,3,4-tetrazol-5-yl)-1H-1,3-benzodiazole (Compound 153)

To a 5 mL microwave tube was added 1-((1H-pyrazol-4-yl)methyl)-2-((4-((2-(4-chlorobenzyl)pyrimidin-4-yl)oxy)piperidin-1-yl)methyl)-1H-benzo[d]imidazole-5-carbonitrile (60 mg, 0.11 mmol) in dioxane (2 mL), DBTO (55 mg, 0.22 mmol) and TMSN₃ (38 mg, 0.33 mmol). The reaction mixture was stirred at 100° C. for 4 hours under N₂ atmosphere. The solvent was removed in vacuo. The residue was purified by Prep-HPLC to afford 2-{[4-({2-[(4-chlorophenyl)methyl]pyrimidin-4-yl}oxy)piperidin-1-yl]methyl}-1-[(1H-pyrazol-4-yl)methyl]-5-(1H-1,2,3,4-tetrazol-5-yl)-1H-1,3-benzodiazole Compound 153 (21 mg, yield: 32%) as a white solid.

MS Calcd.: 581.2; MS Found: 582.3 [M+H]⁺.

¹H NMR (400 MHz, DMSO-d₆): δ 8.50 (d, J=6.0 Hz, 1H), 8.38 (s, 1H), 7.98-8.06 (m, 2H), 7.76 (s, 2H), 7.33-7.38 (m, 4H), 6.82 (d, J=6.0 Hz, 1H), 5.50 (s, 2H), 5.26-5.35 (m, 1H), 4.97 (s, 2H), 4.11 (s, 2H), 3.45-3.70 (m, 4H), 2.20-2.32 (m, 2H), 2.00-2.14 (m, 2H).

### Example 36: 1-[(2-{[4-({2-[(4-chlorophenyl)methyl]pyrimidin-4-yl}oxy)piperidin-1-yl]methyl}-5-[5-(trifluoromethyl)-4H-1,2,4-triazol-3-yl]-1H-1,3-benzodiazol-1-yl)methyl]cyclopropane-1-carbonitrile (Compound 157)

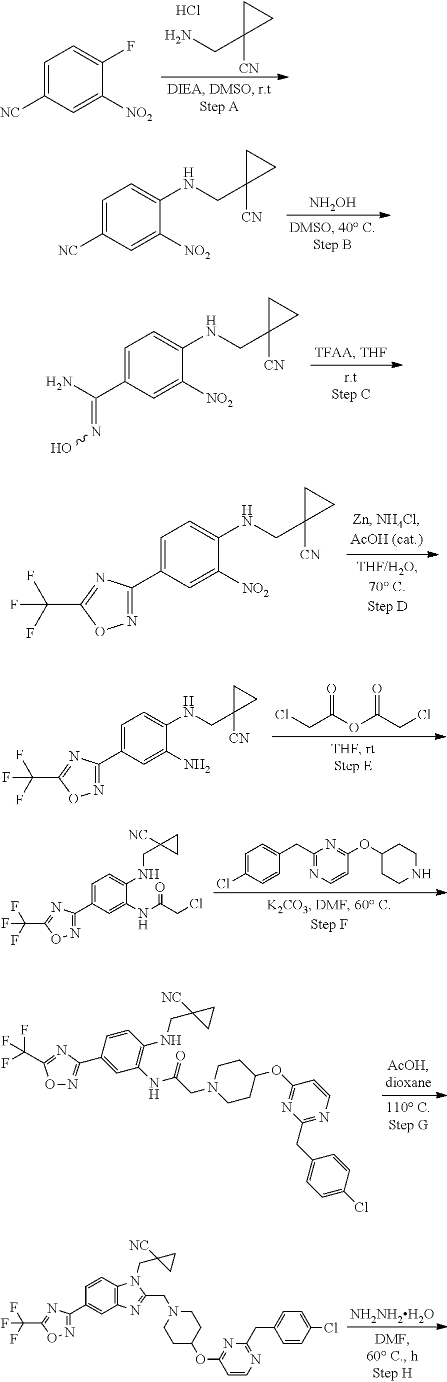

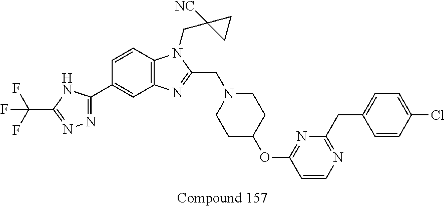 [SMILES: N#CC1(Cn2c(CN3CCC(Oc4ccnc(Cc5ccc(Cl)cc5)n4)CC3)nc3cc(-c4nnc(C(F)(F)F)[nH]4)ccc32)CC1]

### Step A: 4-(((1-cyanocyclopropyl)methyl)amino)-3-nitrobenzonitrile

A mixture of 4-fluoro-3-nitrobenzonitrile (1.0 g, 6.1 mmol), 1-(aminomethyl)cyclopropane-1-carbonitrile (0.8 g, 6.1 mmol, HCl salt) and DIEA (2.3 g, 18.2 mmol) in DMSO (15 mL) was stirred at rt for 2 hours. After the reaction was completed. The mixture was poured into water. The solid was collected by filtration and dried to give 4-(((1-cyanocyclopropyl)methyl)amino)-3-nitrobenzonitrile (1.3 g, 89% yield) as a yellow solid. MS Calcd.: 242.1; MS Found: 241.1 [M+H]⁻.

### Step B: 4-(((1-cyanocyclopropyl)methyl)amino)-N′-hydroxy-3-nitrobenzimidamide

To a solution of 4-(((1-cyanocyclopropyl)methyl)amino)-3-nitrobenzonitrile (1.3 g) in DMSO (15 mL) was added NH₂OH (50 wt. % in water, 1.1 g, 16.1 mmol). The resulting mixture was heated to 40° C. for 2 hours. The reaction was cooled to room temperature. The reaction was diluted with water, extracted with EA. The organic layer was concentrated under vacuum. The residue was purified by column chromatography on silica gel (DCM/MeOH=40/1) to give 4-(((1-cyanocyclopropyl)methyl)amino)-N′-hydroxy-3-nitrobenzimidamide (0.6 g, 40% yield) as a yellow solid. MS Calcd.: 275.1; MS Found: 276.0 [M+H]⁺.

### Step C: 1-(((2-nitro-4-(5-(trifluoromethyl)-1,2,4-oxadiazol-3-yl)phenyl)amino)methyl) cyclopropane-1-carbonitrile

To a solution of 4-(((1-cyanocyclopropyl)methyl)amino)-N′-hydroxy-3-nitrobenzimidamide (270 mg, 0.98 mmol) in THE (10 mL) was added TFAA (618 mg, 2.9 mmol). The solution was stirred at room temperature for 1 hour. The reaction was quenched with Sat. sodium bicarbonate aqueous solution, extracted with ethyl acetate. The organic layers were combined, dried over Na₂SO₄, filtered, and concentrated under vacuum. The residue was purified by column chromatography on silica gel (PE/EA=3/1) to give 1-(((2-nitro-4-(5-(trifluoromethyl)-1,2,4-oxadiazol-3-yl)phenyl)amino)methyl) cyclopropane-1-carbonitrile (200 mg, 58% yield) as a yellow solid. MS Calcd.: 353.1; MS Found: 352.1 [M+H]⁺.

### Step D: 1-(((2-amino-4-(5-(trifluoromethyl)-1,2,4-oxadiazol-3-yl)phenyl)amino)methyl) cyclopropane-1-carbonitrile

To a mixture of 1-(((2-nitro-4-(5-(trifluoromethyl)-1,2,4-oxadiazol-3-yl)phenyl)amino)methyl) cyclopropane-1-carbonitrile (180 mg, 0.51 mmol) in THE (6 mL) and water (1 mL) was added NH₄Cl (275 mg, 5.1 mmol), Zinc powder (166 mg, 2.6 mmol) and AcOH (cat.). The resulting mixture was stirred at 70° C. for 2 hours. The reaction mixture was filtered through celite. The filtrate was diluted with Sat. NaHCO₃ (5 mL) and extracted with ethyl acetate (20 mL*3). The organic layers were combined, dried over Na₂SO₄, filtered, and concentrated to give 1-(((2-amino-4-(5-(trifluoromethyl)-1,2,4-oxadiazol-3-yl)phenyl)amino)methyl) cyclopropane-1-carbonitrile (170 mg, 92% yield) as a yellow solid. MS Calcd.: 323.1; MS Found: 324.1 [M+H]⁺.

### Step E: 2-chloro-N-(2-(((1-cyanocyclopropyl)methyl)amino)-5-(5-(trifluoromethyl)-1,2,4-oxadiazol-3-yl)phenyl)acetamide

A mixture of 1-(((2-amino-4-(5-(trifluoromethyl)-1,2,4-oxadiazol-3-yl)phenyl)amino)methyl) cyclopropane-1-carbonitrile (180 mg, 0.56 mmol) and 2-chloroacetic anhydride (105 mg, 0.61 mmol) in THE (5 mL) was stirred at room temperature for 12 hours. The reaction was diluted with water, extracted with EA. The organic layers were combined, dried over Na₂SO₄, filtered, and concentrated to give 2-chloro-N-(2-(((1-cyanocyclopropyl)methyl)amino)-5-(5-(trifluoromethyl)-1,2,4-oxadiazol-3-yl)phenyl)acetamide (190 mg, 81% yield) as a yellow solid. MS Calcd.: 399.1; MS Found: 400.1 [M+H]⁺.

### Step F: 2-(4-((2-(4-chlorobenzyl)pyrimidin-4-yl)oxy)piperidin-1-yl)-N-(2-(((1-cyanocyclopropyl)methyl)amino)-5-(5-(trifluoromethyl)-1,2,4-oxadiazol-3-yl)phenyl)acetamide

To a solution of 2-chloro-N-(2-(((1-cyanocyclopropyl)methyl)amino)-5-(5-(trifluoromethyl)-1,2,4-oxadiazol-3-yl)phenyl)acetamide (90 mg, 0.23 mmol) in DMF (5 mL) was added K₂CO₃ (190 mg, 1.4 mmol) and 2-(4-chlorobenzyl)-4-(piperidin-4-yloxy)pyrimidine (310 mg, 0.90 mmol). The resulting mixture was heated to 60° C. for 1 hour. The reaction was quenched with water, extracted with ethyl acetate. The organic layers were combined, dried over Na₂SO₄, filtered, and concentrated under vacuum. The residue was purified by column chromatography on silica gel (DCM/MeOH=30/1) to give 2-(4-((2-(4-chlorobenzyl)pyrimidin-4-yl)oxy)piperidin-1-yl)-N-(2-(((1-cyanocyclopropyl)methyl)amino)-5-(5-(trifluoromethyl)-1,2,4-oxadiazol-3-yl)phenyl)acetamide (110 mg, 73% yield) as a yellow oil. MS Calcd.: 666.2; MS Found: 667.1 [M+H]⁺.

### Step G: 1-((2-((4-((2-(4-chlorobenzyl)pyrimidin-4-yl)oxy)piperidin-1-yl)methyl)-5-(5-(trifluoromethyl)-1,2,4-oxadiazol-3-yl)-1H-benzo[d]imidazol-1-yl)methyl)cyclopropane-1-carbonitrile

A mixture of 2-(4-((2-(4-chlorobenzyl)pyrimidin-4-yl)oxy)piperidin-1-yl)-N-(2-(((1-cyanocyclopropyl) methyl)amino)-5-(5-(trifluoromethyl)-1,2,4-oxadiazol-3-yl)phenyl)acetamide (90 mg, 0.14 mmol) in dioxane (4.0 mL) and AcOH (0.1 mL) was heated to 110° C. for 4 hours, quenched with Sat. sodium bicarbonate aqueous solution (3 mL) and extracted with ethyl acetate. The organic layers were combined, dried over Na₂SO₄, filtered, and concentrated under vacuum. The residue was purified by column chromatography on silica gel (DCM/MeOH=30/1) to give 1-((2-((4-((2-(4-chlorobenzyl)pyrimidin-4-yl)oxy)piperidin-1-yl)methyl)-5-(5-(trifluoromethyl)-1,2,4-oxadiazol-3-yl)-1H-benzo[d]imidazol-1-yl)methyl)cyclopropane-1-carbonitrile (50 mg, 57% yield) as a white solid. MS Calcd.: 648.2; MS Found: 649.2 [M+H]⁺.

### Step H: 1-[(2-{[4-({2-[(4-chlorophenyl)methyl]pyrimidin-4-yl}oxy)piperidin-1-yl]methyl}-5-[5-(trifluoromethyl)-4H-1,2,4-triazol-3-yl]-1H-1,3-benzodiazol-1-yl)methyl]cyclopropane-1-carbonitrile

To a solution of 1-((2-((4-((2-(4-chlorobenzyl)pyrimidin-4-yl)oxy)piperidin-1-yl)methyl)-5-(5-(trifluoromethyl)-1,2,4-oxadiazol-3-yl)-1H-benzo[d]imidazol-1-yl)methyl)cyclopropane-1-carbonitrile (50 mg, 0.077 mmol) in DMF (2.0 mL) was added hydrazine hydrate (12 mg, 0.38 mmol). The solution was stirred at 60° C. for 1 hour. The reaction solution was filtered through the filter membrane, the filtrate was purified by Prep-HPLC (0.1% TFA) to give 1-[(2-{[4-({2-[(4-chlorophenyl)methyl]pyrimidin-4-yl}oxy)piperidin-1-yl]methyl}-5-[5-(trifluoromethyl)-4H-1,2,4-triazol-3-yl]-1H-1,3-benzodiazol-1-yl)methyl]cyclopropane-1-carbonitrile Compound 157 (9 mg, 19% yield) as a white solid. MS Calcd.: 647.2; MS Found: 648.3 [M+H]⁺.

¹H NMR (400 MHz, DMSO-d₆): δ 15.34 (s, 1H), 8.48 (d, J=5.6 Hz, 1H), 8.41 (s, 1H), 8.00-8.10 (m, 2H), 7.30-7.40 (m, 4H), 6.79 (d, J=5.6 Hz, 1H), 5.32-5.21 (m, 1H), 4.85-4.95 (m, 2H), 4.70 (s, 2H), 4.10 (m, 2H), 3.29-3.52 (m, 4H), 2.15-2.28 (m, 2H), 1.93-2.12 (m, 2H), 1.40-1.45 (m, 4H). ¹⁹F-NMR (377 MHz): −63.78.

### Example 37: 1-[(2-{[4-({2-[(4-chlorophenyl)methyl]pyrimidin-4-yl}oxy)piperidin-1-yl]methyl}-5-(5-oxo-4,5-dihydro-1,2,4-oxadiazol-3-yl)-1H-1,3-benzodiazol-1-yl)methyl]cyclopropane-1-carbonitrile (Compound 155)

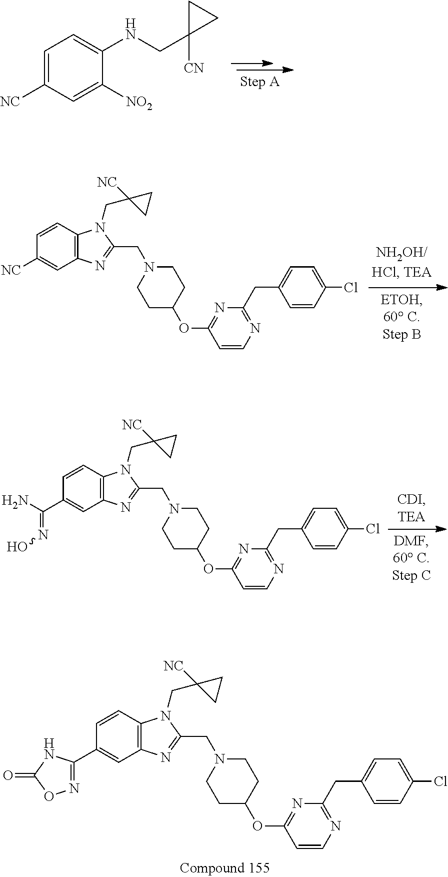 [SMILES: N#Cc1ccc(NCC2(C#N)CC2)c([N+](=O)[O-])c1 ; N#Cc1ccc2c(c1)nc(CN1CCC(Oc3ccnc(Cc4ccc(Cl)cc4)n3)CC1)n2CC1(C#N)CC1 ; N#CC1(Cn2c(CN3CCC(Oc4ccnc(Cc5ccc(Cl)cc5)n4)CC3)nc3cc(C(N)=NO)ccc32)CC1 ; N#CC1(Cn2c(CN3CCC(Oc4ccnc(Cc5ccc(Cl)cc5)n4)CC3)nc3cc(-c4noc(=O)[nH]4)ccc32)CC1]

### Step A: 2-((4-((2-(4-chlorobenzyl)pyrimidin-4-yl)oxy)piperidin-1-yl)methyl)-1-((1-cyanocyclopropyl)methyl)-1H-benzo[d]imidazole-5-carbonitrile

Starting from 4-(((1-cyanocyclopropyl)methyl)amino)-3-nitrobenzonitrile, 2-((4-((2-(4-chlorobenzyl)pyrimidin-4-yl)oxy)piperidin-1-yl)methyl)-1-((1-cyanocyclopropyl)methyl)-1H-benzo[d]imidazole-5-carbonitrile (300 mg) was obtained with the similar method for Compound 106a as a yellow solid. MS Calcd.: 537.2; MS Found: 538.2 [M+H]⁺.

### Step B: 2-((4-((2-(4-chlorobenzyl)pyrimidin-4-yl)oxy)piperidin-1-yl)methyl)-1-((1-cyanocyclopropyl)methyl)-N′-hydroxy-1H-benzo[d]imidazole-5-carboximidamide

A mixture of 2-((4-((2-(4-chlorobenzyl)pyrimidin-4-yl)oxy)piperidin-1-yl)methyl)-1-((1-cyanocyclopropyl)methyl)-1H-benzo[d]imidazole-5-carbonitrile (200.1 mg, 0.37 mmol), NH₂OH·HCl (13.6 mg, 0.44 mmol), TEA (112.1 mg, 1.11 mmol) in EtOH (2 mL) was stirred at 60° C. for 3 hours. After the reaction was completed, the mixture was concentrated. The residue was purified by column chromatography on silica gel (DCM/MeOH=12/1) to give 2-((4-((2-(4-chlorobenzyl)pyrimidin-4-yl)oxy)piperidin-1-yl)methyl)-1-((1-cyanocyclopropyl)methyl)-N′-hydroxy-1H-benzo[d]imidazole-5-carboximidamide (60 mg, yield: 28%). MS Calcd.: 570.2; MS Found: 571.2 [M+H]⁺.

### Step C: 1-[(2-{[4-({2-[(4-chlorophenyl)methyl]pyrimidin-4-yl}oxy)piperidin-1-yl]methyl}-5-(5-oxo-4,5-dihydro-1,2,4-oxadiazol-3-yl)-1H-1,3-benzodiazol-1-yl)methyl]cyclopropane-1-carbonitrile

A mixture of 2-((4-((2-(4-chlorobenzyl)pyrimidin-4-yl)oxy)piperidin-1-yl)methyl)-1-((1-cyanocyclopropyl)methyl)-N′-hydroxy-1H-benzo[d]imidazole-5-carboximidamide (60 mg, 0.11 mmol), CDI (27.5 mg, 0.17 mmol) and TEA (33.3 mg, 0.33 mmol) in DMF (1 mL) was stirred at 60° C. for 3 hours. After the reaction was completed, the reaction mixture was purified directly by Prep-HPLC to give 1-[(2-{[4-({2-[(4-chlorophenyl)methyl]pyrimidin-4-yl}oxy)piperidin-1-yl]methyl}-5-(5-oxo-4,5-dihydro-1,2,4-oxadiazol-3-yl)-1H-1,3-benzodiazol-1-yl)methyl]cyclopropane-1-carbonitrile Compound 155 (4 mg, yield: 7%).

MS Calcd.: 596.2; MS Found: 597.3 [M+H]+

¹H NMR (400 MHz, DMSO-d₆): δ 8.41 (d, J=6.0 Hz, 1H), 8.07 (s, 1H), 7.86 (d, J=8.0 Hz, 1H), 7.75 (d, J=8.8 Hz, 1H), 7.30-7.36 (m, 4H), 6.73 (d, J=6.0 Hz, 1H), 4.97-5.08 (m, 1H), 4.69 (s, 2H), 4.08 (s, 2H), 3.92 (s, 2H), 2.65-2.80 (m, 2H), 2.31-2.40 (m, 2H), 1.88-1.97 (m, 2H), 1.60-1.70 (m, 2H), 1.38-1.42 (m, 4H).

### Example 38: 3-(2-{[4-({2-[(4-chlorophenyl)methyl]pyrimidin-4-yl}oxy)piperidin-1-yl]methyl}-3-{[(2S)-oxetan-2-yl]methyl}-3H-imidazo[4,5-c]pyridin-6-yl)-4,5-dihydro-1,2,4-oxadiazol-5-one (Compound 145a)

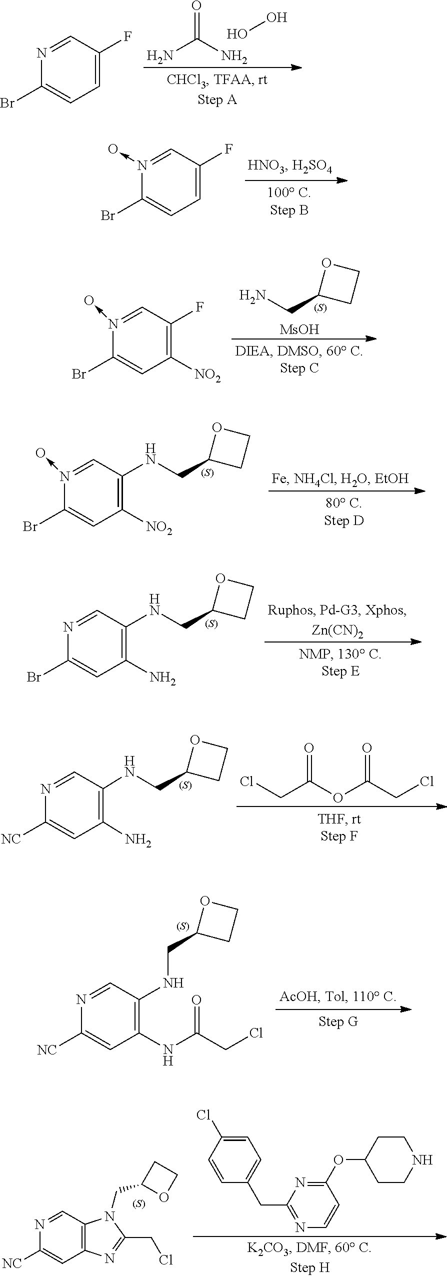

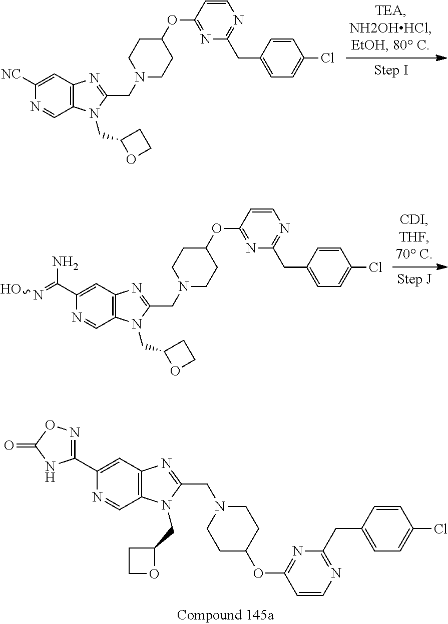 [SMILES: N#Cc1cc2nc(CN3CCC(Oc4ccnc(Cc5ccc(Cl)cc5)n4)CC3)n(C[C@@H]3CCO3)c2cn1 ; NC(=NO)c1cc2nc(CN3CCC(Oc4ccnc(Cc5ccc(Cl)cc5)n4)CC3)n(C[C@@H]3CCO3)c2cn1 ; O=c1[nH]c(-c2cc3nc(CN4CCC(Oc5ccnc(Cc6ccc(Cl)cc6)n5)CC4)n(C[C@@H]4CCO4)c3cn2)no1]

### Step A: 2-bromo-5-fluoropyridine 1-oxide

To a solution of 2-bromo-5-fluoropyridine (5.0 g, 28.4 mmol), Urea peroxide (3.5 g, 36.9 mmol) in CHCl₃ (50 mL) was added TFAA (11.9 g, 56.8 mmol) at 0° C. The reaction mixture was heated to room temperature for 3 hours. The reaction was quenched with NaHSO₃, extracted with ethyl acetate. The organic layers were combined, dried over Na₂SO₄, filtered, and concentrated under vacuum. The residue was purified by silica gel column and eluted with PE:EtOAc=10:1 to give 2-bromo-5-fluoropyridine 1-oxide (4.9 mg, 91% yield) as a white solid. MS Calcd.: 190.9; MS Found: 191.8 [M+H]⁺.

### Step B: 2-bromo-5-fluoro-4-nitropyridine 1-oxide

To a solution of 2-bromo-5-fluoropyridine 1-oxide (3.0 g, 15.7 mmol) in H₂SO₄ (52 mL) was added HNO₃ (26 mL) at room temperature. The reaction mixture was heated to 100° C. for 4 hours. The reaction was poured into ice water slowly, pH value was adjusted to 7 with Na₂CO₃ and extracted with EA. The organic layers were combined, dried over Na₂SO₄, filtered, and concentrated under vacuum. The residue was purified by silica gel column and eluted with PE:EtOAc=5:1 to give 2-bromo-5-fluoro-4-nitropyridine 1-oxide (3.0 g, 93% yield) as a yellow solid.

### Step C: (S)-2-bromo-4-nitro-5-((oxetan-2-ylmethyl)amino)pyridine 1-oxide

To a solution of 2-bromo-5-fluoro-4-nitropyridine 1-oxide (500 mg, 2.1 mmol) in DMSO (8 mL) was added (S)-oxetan-2-ylmethanamine methanesulfonate (465 mg, 2.5 mmol) and DIEA (546 mg, 4.2 mmol). The reaction mixture was stirred at 60° C. for 2 hours. The reaction was quenched with water, extracted with ethyl acetate. The organic layers were combined, dried over Na₂SO₄, filtered, and concentrated under vacuum. The residue was purified by silica gel column and eluted with PE:EtOAc=1:1 to give (S)-2-bromo-4-nitro-5-((oxetan-2-ylmethyl)amino)pyridine 1-oxide (510 mg, 79% yield) as a yellow solid. MS Calcd.: 303.0; MS Found: 303.9 [M+H]⁺.

### Step D: (S)-6-bromo-N³-(oxetan-2-ylmethyl)pyridine-3,4-diamine

To a solution of (S)-2-bromo-4-nitro-5-((oxetan-2-ylmethyl)amino)pyridine 1-oxide (510 mg, 1.65 mmol) in EtOH (10 mL) and water (2 mL) was added ammonium chloride (875 mg, 16.5 mmol) and iron powder (924 mg, 16.5 mmol). The solution was stirred at 80° C. for 4 hours. The reaction mixture was filtered through a celite pad. The filtrate was concentrated under vacuum. The residue was washed with water and ethyl acetate. The organic layer was dried over Na₂SO₄, filtered, and concentrated under vacuum to give (S)-6-bromo-N³-(oxetan-2-ylmethyl)pyridine-3,4-diamine (300 mg, 70% yield). MS Calcd.: 257.0; MS Found: 299.0 [M+H+41]⁺.

### Step E: (S)-4-amino-5-((oxetan-2-ylmethyl)amino)picolinonitrile

A mixture of (S)-6-bromo-N3-(oxetan-2-ylmethyl)pyridine-3,4-diamine (300 mg, 1.17 mmol), Ruphos Pd-G3 (195 mg, 0.23 mmol), X-phos (112 mg, 0.23 mmol) and Zn(CN)₂ (1.64 g, 14.04 mmol) in NMP (6.0 mL) was stirred at 130° C. for 5 minutes. After the reaction was completed, the mixture was filtered and diluted with with EA (50 mL)/brine (50 mL). The organic layer was concentrated under vacuum. The residue was purified by column chromatography on silica gel (DCM/MeOH=30/1) to give (S)-4-amino-5-((oxetan-2-ylmethyl)amino)picolinonitrile (260 mg, crude) as colorless oil. MS Calcd.: 204.1; MS Found: 205.2[M+H]⁺.

### Step F: (S)-2-chloro-N-(2-cyano-5-((oxetan-2-ylmethyl)amino)pyridin-4-yl)acetamide

A mixture of (S)-4-amino-5-((oxetan-2-ylmethyl)amino)picolinonitrile (260 mg, crude) and 2-chloroacetic anhydride (238 mg, 1.4 mmol) in THE (3.0 mL) was stirred at room temperature for 16 hours. The reaction mixture was directly purified by column chromatography on silica gel (PE/EA=1/3) to give (S)-2-chloro-N-(2-cyano-5-((oxetan-2-ylmethyl)amino)pyridin-4-yl)acetamide (200 mg, 56% yield) as a white solid. MS Calcd.: 280.1; MS Found: 281.1 [M+H]⁺.

### Step G: (S)-2-(chloromethyl)-3-(oxetan-2-ylmethyl)-3H-imidazo[4,5-c]pyridine-6-carbonitrile

A mixture of (S)-2-(chloromethyl)-3-(oxetan-2-ylmethyl)-3H-imidazo[4,5-c]pyridine-6-carbonitrile (180 mg, 0.64 mmol) in toluene (5.0 mL) and AcOH (0.2 mL) was heated to 110° C. for 8 hours. After the reaction was cooled to room temperature, NaHCO₃ (sat.) (4.0 mL) was added and extracted with ethyl acetate. The organic layers were combined, dried over Na₂SO₄, filtered and concentrated under vacuum. The residue was purified by column chromatography on silica gel (DCM/MeOH=50/1) to give (S)-2-(chloromethyl)-3-(oxetan-2-ylmethyl)-3H-imidazo[4,5-c]pyridine-6-carbonitrile (110 mg, 65% yield) as a yellow solid. MS Calcd.: 262.2; MS Found: 263.1 [M+H]⁺.

### Step H: (S)-2-((4-((2-(4-chlorobenzyl)pyrimidin-4-yl)oxy)piperidin-1-yl)methyl)-6-isocyano-3-(oxetan-2-ylmethyl)-3H-imidazo[4,5-c]pyridine

To a solution of 2-(4-chlorobenzyl)-4-(piperidin-4-yloxy)pyrimidine (185 mg, 0.61 mmol) in DMF (5 mL) was added K₂CO₃ (128 mg, 0.93 mmol) and (S)-2-(chloromethyl)-6-isocyano-3-(oxetan-2-ylmethyl)-3H-imidazo[4,5-c]pyridine (80 mg, 0.31 mmol). The resulting mixture was stirred at 60° C. for 1 hour. After the reaction was completed, the mixture was diluted with EA (50 mL), washed with H₂O (40 mL). The organic layer was dried over anhydrous sodium sulfate, filtered, and evaporated to dryness. The residue was purified by column chromatography on silica gel (DCM/MeOH=1/50) to give (S)-2-((4-((2-(4-chlorobenzyl)pyrimidin-4-yl)oxy)piperidin-1-yl)methyl)-6-isocyano-3-(oxetan-2-ylmethyl)-3H-imidazo[4,5-c]pyridine (120 mg, yield: 75%) as yellow oil. MS Calcd.: 529.2; MS Found: 530.2 [M+H]⁺.

### Step I: (S)-2-((4-((2-(4-chlorobenzyl)pyrimidin-4-yl)oxy)piperidin-1-yl)methyl)-N′-hydroxy-3-(oxetan-2-ylmethyl)-3H-imidazo[4,5-c]pyridine-6-carboximidamide

To a solution of (S)-2-((4-((2-(4-chlorobenzyl)pyrimidin-4-yl)oxy)piperidin-1-yl)methyl)-6-isocyano-3-(oxetan-2-ylmethyl)-3H-imidazo[4,5-c]pyridine (120 mg, 0.23 mmol) in EtOH (3 mL) was added TEA (91.8 mg, 0.91 mmol) and NH₂OH HCl (95.95 mg, 1.38 mmol). The resulting mixture was stirred at 80° C. for 1 hour. After the reaction was completed, the mixture was diluted with EA (30 mL), washed with H₂O (20 mL). The organic layer was dried over anhydrous sodium sulfate, filtered, and evaporated to dryness. The reaction mixture was concentrated to give (S)-2-((4-((2-(4-chlorobenzyl)pyrimidin-4-yl)oxy)piperidin-1-yl)methyl)-N′-hydroxy-3-(oxetan-2-ylmethyl)-3H-imidazo[4,5-c]pyridine-6-carboximidamide (90 mg, yield: 70%) as a white solid. MS Calcd.: 562.2; MS Found: 563.2 [M+H]⁺.

### Step J: 3-(2-{[4-({2-[(4-chlorophenylmethyl]pyrimidin-4-yl}oxy)piperidin-1-yl]methyl}-3-{1[(2S)-oxetan-2-yl]methyl}-3H-imidazo[4,5-c]pyridin-6-yl)-4,5-dihydro-1,2,4-oxadiazol-5-one (Compound 145a)

To a solution of (S)-2-((4-((2-(4-chlorobenzyl)pyrimidin-4-yl)oxy)piperidin-1-yl)methyl)-N′-hydroxy-3-(oxetan-2-ylmethyl)-3H-imidazo[4,5-c]pyridine-6-carboximidamide (30 mg) in THE (2 mL) was added TEA (16 mg, 0.16 mmol) and CDI (13 mg, 0.08 mmol). The resulting mixture was stirred at 70° C. for 12 hours. The solvent was removed in vacuo. The residue was purified by Prep-HPLC to give 3-(2-{[4-({2-[(4-chlorophenyl)methyl]pyrimidin-4-yl}oxy)piperidin-1-yl]methyl}-3-{[(2S)-oxetan-2-yl]methyl}-3H-imidazo[4,5-c]pyridin-6-yl)-4,5-dihydro-1,2,4-oxadiazol-5-one Compound 145a (11 mg, yield: 35%) as a white solid.

MS Calcd.: 588.2; MS Found: 589.2 [M+H]⁺.

¹H NMR (400 MHz, DMSO-d₆): δ 12.99 (br s, 1H), 9.16 (s, 1H), 8.41 (d, J=6.0 Hz, 1H), 8.18 (s, 1H), 7.31-7.38 (m, 4H), 6.72 (d, J=6.0 Hz, 1H), 5.08-5.17 (m, 1H), 4.98-5.07 (m, 1H), 4.85-4.95 (m, 1H), 4.70-4.76 (m, 1H), 4.47-4.55 (m, 1H), 4.38-4.44 (m, 1H), 4.08 (s, 2H), 3.95-4.10 (m, 1H), 3.80-3.95 (m, 1H), 2.65-2.85 (m, 3H), 2.35-2.50 (m, 3H), 1.88-1.97 (m, 2H), 1.58-1.70 (m, 2H).

### Example 39: 2-[(4-chlorophenyl)methyl]-5-fluoro-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine (Compound 149a)

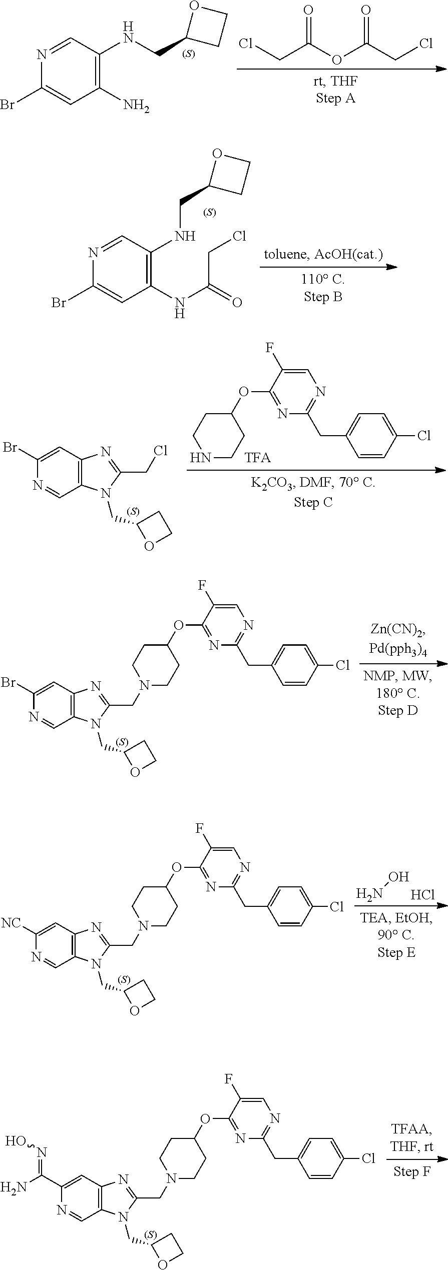

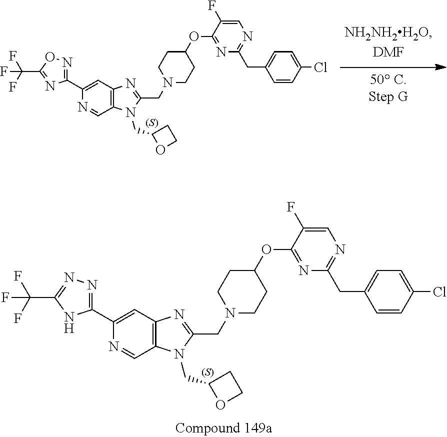

### Step A: (S)—N-(2-bromo-5-((oxetan-2-ylmethyl)amino)pyridin-4-yl)-2-chloroacetamide

To a solution of (S)-6-bromo-N³-(oxetan-2-ylmethyl)pyridine-3,4-diamine (600 mg, 2.3 mmol) in THF (10 mL) was added 2-chloroacetic anhydride (439 mg, 2.6 mmol). The resulting mixture was stirred at room temperature for 12 hours. The solvent was removed in vacuo. The residue was purified by silica gel column and eluted with PE:EtOAc=1:2 to give (S)—N-(2-bromo-5-((oxetan-2-ylmethyl)amino)pyridin-4-yl)-2-chloroacetamide (500 mg, 64% yield) as a white solid. MS Calcd.: 333.0; MS Found: 334.0 [M+H]⁺.

### Step B: (S)-6-bromo-2-(chloromethyl)-3-(oxetan-2-ylmethyl)-3H-imidazo[4,5-c]pyridine

A mixture of (S)—N-(2-bromo-5-((oxetan-2-ylmethyl)amino)pyridin-4-yl)-2-chloroacetamide (1.5 g, 4.5 mmol) in toluene (20 mL) and AcOH (0.2 mL) was heated to 110° C. for 16 hours. After cooled down to room temperature, the reaction mixture was quenched with Sat. aqueous NaHCO₃ (1 mL), diluted with water (10 mL). The organic layer was separated and evaporated in vacuo to give (S)-6-bromo-2-(chloromethyl)-3-(oxetan-2-ylmethyl)-3H-imidazo[4,5-c]pyridine (1.1 g, 85% purity, 65% yield) as a brown solid. The crude product was used in the next step directly without purification. MS Calcd.: 315.0; MS Found: 316.0 [M+H]⁺.

### Step C: (S)-6-bromo-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine

To a solution of 2-(4-chlorobenzyl)-5-fluoro-4-(piperidin-4-yloxy)pyrimidine (410 mg, 0.95 mmol) in DMF (6 mL) was added K₂CO₃ (391 mg, 2.8 mmol). After a few minutes, (S)-6-bromo-2-(chloromethyl)-3-(oxetan-2-ylmethyl)-3H-imidazo[4,5-c]pyridine (300 mg, 0.95 mmol) was added. The resulting mixture was heated to 70° C. for an hour. The reaction was quenched with water, extracted with ethyl acetate (20 mL×3). The organic layers were combined, dried over Na₂SO₄, filtered, and concentrated under vacuum. The residue was purified by silica column chromatography, eluting with DCM:MeOH=20:1 to give (S)-6-bromo-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine (500 mg, 88% yield) as an off-white solid. MS Calcd.: 600.1; MS Found: 601.1 [M+H]⁺.

### Step D: (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine-6-carbonitrile

To a solution of (S)-6-bromo-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine (500 mg, 0.83 mmol) in NMP (8 mL) was added Zn(CN)₂ (58 mg, 0.50 mmol) and Pd(PPh₃)₄ (144 mg, 0.12 mmol). The reaction mixture was irradiated with microwave radiation for 30 minutes at 180° C. The reaction was quenched with water, extracted with ethyl acetate (20 mL×3). The organic layers were combined, dried over Na₂SO₄, filtered, and concentrated under vacuum. The residue was purified by silica gel chromatography, eluting with DCM:MeOH=25:1 to give (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine-6-carbonitrile (280 mg, 61% yield) as brown oil. MS Calcd.: 547.2; MS Found: 548.2 [M+H]⁺.

### Step E: (S)-2-((4-((2-(4-chlorobenzyl)-5-fluoropyrimidin-4-yl)oxy)piperidin-1-yl)methyl)-N′-hydroxy-3-(oxetan-2-ylmethyl)-3H-imidazo[4,5-c]pyridine-6-carboximidamide

To a solution of hydroxylamine hydrochloride (71 mg, 1.0 mmol) in EtOH (5 mL) was added TEA (155 mg, 1.5 mmol). After 2 minutes, (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine-6-carbonitrile (280 mg, 0.51 mmol) was added. The resulting mixture was heated to 90° C. for 2 hours. The reaction was cooled to room temperature. The solid was collected by filtration and dried to give (S)-2-((4-((2-(4-chlorobenzyl)-5-fluoropyrimidin-4-yl)oxy)piperidin-1-yl)methyl)-N′-hydroxy-3-(oxetan-2-ylmethyl)-3H-imidazo[4,5-c]pyridine-6-carboximidamide (130 mg, 43% yield) as a white solid. MS Calcd.: 580.2; MS Found: 581.2 [M+H]⁺.

### Step F: (S)-3-(2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridin-6-yl)-5-(trifluoromethyl)-1,2,4-oxadiazole

To a solution of (S)-2-((4-((2-(4-chlorobenzyl)-5-fluoropyrimidin-4-yl)oxy)piperidin-1-yl)methyl)-N′-hydroxy-3-(oxetan-2-ylmethyl)-3H-imidazo[4,5-c]pyridine-6-carboximidamide (70 mg) in THE (1.5 mL) was added TFAA (101 mg, 0.48 mmol). The solution was stirred at room temperature for an hour. The reaction was quenched with sodium bicarbonate aqueous solution, extracted with ethyl acetate. The organic layers were combined, dried over Na₂SO₄, filtered and concentrated under vacuum to give (S)-3-(2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridin-6-yl)-5-(trifluoromethyl)-1,2,4-oxadiazole (58 mg, 87% purity, 63% yield) as a brown solid. MS Calcd.: 658.2; MS Found: 659.2 [M+H]⁺.

### Step G: 2-[(4-chlorophenyl)methyl]-5-fluoro-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine

To a solution of (S)-3-(2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridin-6-yl)-5-(trifluoromethyl)-1,2,4-oxadiazole (58 mg, 0.088 mmol) in DMF (1.5 mL) was added hydrazine hydrate (18 mg, 0.35 mmol). The solution was stirred at 50° C. for 2 hours. The reaction solution was filtered, the filtrate was purified by Prep-HPLC to give 2-[(4-chlorophenyl)methyl]-5-fluoro-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine (Compound 149a) (14 mg, yield: 24%) as a white solid.

MS Calcd.: 657.2; MS Found: 658.3 [M+H]⁺.

¹H NMR (400 MHz, DMSO-d₆): δ 15.52 (s, 1H), 9.31 (d, J=0.8 Hz, 1H), 8.57 (d, J=3.2 Hz, 1H), 8.39 (d, J=0.8 Hz, 1H), 7.30-7.38 (m, 4H), 5.28-5.41 (m, 1H), 5.07-5.15 (m, 1H), 4.80-4.97 (m, 3H), 4.73-4.80 (m, 1H), 4.48-4.56 (m, 1H), 4.38-4.45 (m, 1H), 4.10 (s, 2H), 3.25-3.60 (m, 4H), 2.72-2.82 (m, 1H), 2.31-2.42 (m, 1H), 2.18-2.30 (m, 2H), 1.95-2.17 (m, 2H). ¹⁹F-NMR (377 MHz): −63.71, −157.07.

### Example 40: 2-[(4-chlorophenyl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 140a)

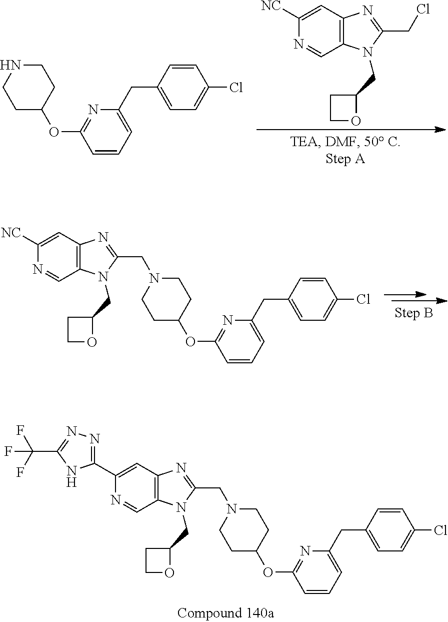 [SMILES: Clc1ccc(Cc2cccc(OC3CCNCC3)n2)cc1 ; N#Cc1cc2nc(CCl)n(C[C@@H]3CCO3)c2cn1 ; N#Cc1cc2nc(CN3CCC(Oc4cccc(Cc5ccc(Cl)cc5)n4)CC3)n(C[C@@H]3CCO3)c2cn1 ; FC(F)(F)c1nnc(-c2cc3nc(CN4CCC(Oc5cccc(Cc6ccc(Cl)cc6)n5)CC4)n(C[C@@H]4CCO4)c3cn2)[nH]1]

### Step A: (S)-2-((4-((6-(4-chlorobenzyl)pyridin-2-yl)oxy)piperidin-1-yl)methyl)-3-(oxetan-2-ylmethyl)-3H-imidazo[4,5-c]pyridine-6-carbonitrile

A mixture of 2-(4-chlorobenzyl)-6-(piperidin-4-yloxy)pyridine (52 mg, 0.15 mmol), (S)-2-(chloromethyl)-3-(oxetan-2-ylmethyl)-3H-imidazo[4,5-c]pyridine-6-carbonitrile (40 mL, 0.15 mmol) and TEA (46 mg, 0.46 mmol) in DMF (1 mL) was stirred at 50° C. for 2 hours. The reaction mixture was quenched with H₂O (25 mL) and extracted with EA (30 mL×3). The combined organic layer was dried over anhydrous Na₂SO₄, filtered and concentrated. The residue was purified by column chromatography on silica gel (DCM:MeOH=20:1) to give (S)-2-((4-((6-(4-chlorobenzyl)pyridin-2-yl)oxy)piperidin-1-yl)methyl)-3-(oxetan-2-ylmethyl)-3H-imidazo[4,5-c]pyridine-6-carbonitrile (80 mg) as a white solid. MS Calcd.: 528.2; MS Found: 529.2 [M+H]⁺.

### Step B: 2-[(4-chlorophenyl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine

2-[(4-chlorophenyl)methyl]-6-({1-[(3-{[(2S)-oxetan-2-yl]methyl}-6-[5-(trifluoro methyl)-4H-1,2,4-triazol-3-yl]-3H-imidazo[4,5-c]pyridin-2-yl)methyl]piperidin-4-yl}oxy)pyridine (Compound 140a) (21 mg) was obtained with the similar procedure of Compound 149a as a white solid.

MS Calcd.: 638.2; MS Found: 639.4 [M+H]⁺.

¹H NMR (400 MHz, DMSO-d₆): δ 9.18 (s, 1H), 8.30 (s, 1H), 7.56-7.64 (m, 1H), 7.28-7.40 (m, 4H), 6.83 (d, J=6.8 Hz, 1H), 6.59 (d, J=8.8 Hz, 1H), 5.10-5.20 (m, 1H), 4.88-5.04 (m, 2H), 4.73-4.80 (m, 1H), 4.48-4.57 (m, 1H), 4.38-4.47 (m, 1H), 3.80-4.08 (m, 4H), 2.60-2.90 (m, 3H), 2.30-2.51 (m, 3H), 1.85-2.04 (m, 2H), 1.50-1.70 (m, 2H). ¹⁹F-NMR (377 MHz): −63.72.

### Example 41: 2-[(4-chlorophenyl)methyl]-5-fluoro-4-[(1-{[6-(5-methyl-4H-1,2,4-triazol-3-yl)-3-{[(2S)-oxetan-2-yl]methyl}-3H-imidazo[4,5-b]pyridin-2-yl]methyl}piperidin-4-yl)oxy]pyrimidine (Compound 162a)

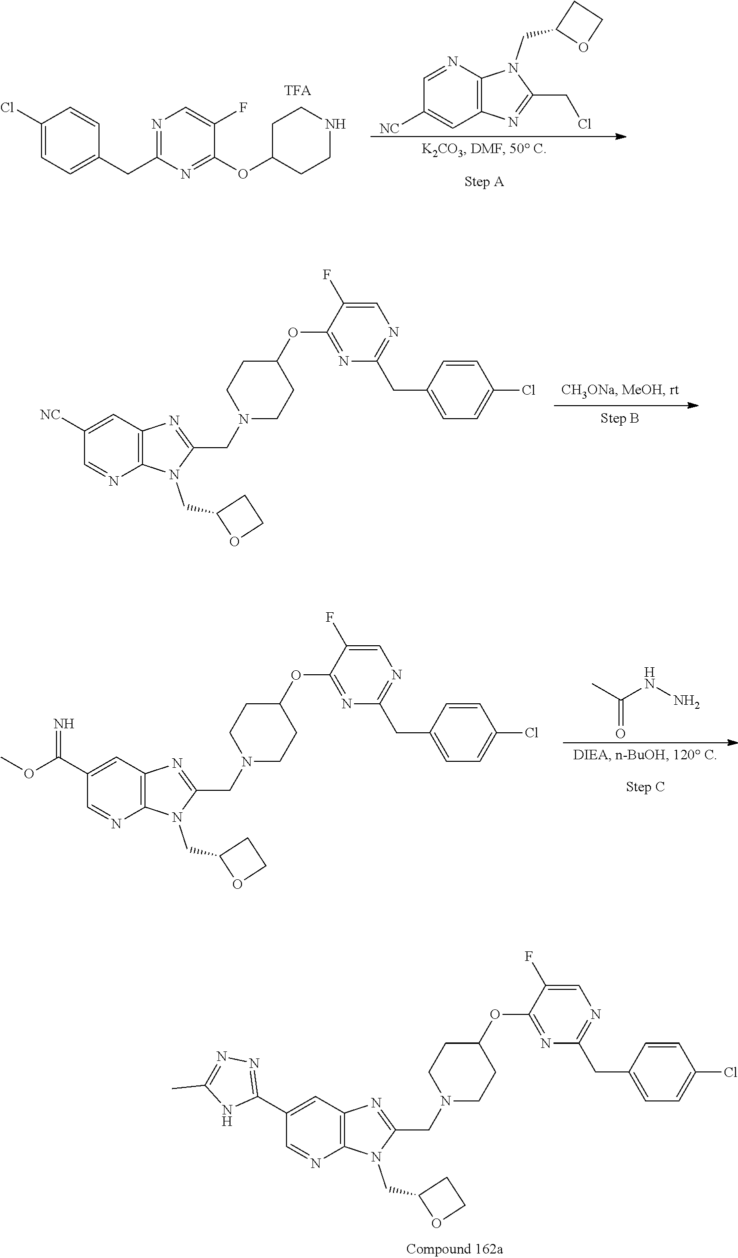

### Step A: (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-b]pyridine-6-carbonitrile

To a solution of 2-(4-chlorobenzyl)-5-fluoro-4-(piperidin-4-yloxy)pyrimidine (196 mg, 0.15 mmol) in DMF (2 mL) was added K₂CO₃ (62.1 mg, 0.45 mmol) and (S)-2-(chloromethyl)-3-(oxetan-2-ylmethyl)-3H-imidazo[4,5-b]pyridine-6-carbonitrile (40 mg, 0.15 mmol). The resulting mixture was stirred at 50° C. for 1 hour. The reaction was filtered, and the filtrate was concentrated to give (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-b]pyridine-6-carbonitrile (70 mg, yield: 84%) as yellow oil. MS Calcd.: 547.2; MS Found: 548.3 [M+H]⁺.

### Step B: methyl (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-b]pyridine-6-carbimidate

A mixture of (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-b]pyridine-6-carbonitrile (60 mg), MeONa (60 mg, 1.1 mmol) in MeOH (2 mL) was stirred at room temperature for 5 h. After the reaction was completed, the mixture was concentrated in vacuum. The residue was purified by Prep-TLC (MeOH/DCM=1/20) to afford methyl (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-b]pyridine-6-carbimidate (20 mg, yield: 32%) as yellow oil. MS Calcd.: 579.2; MS Found: 580.2 [M+H]+.

### Step C: 2-[(4-chlorophenyl)methyl]-5-fluoro-4-[(1-{[6-(5-methyl-4H-1,2,4-triazol-3-yl)-3-{[(2S)-oxetan-2-yl]methyl}-3H-imidazo[4,5-b]pyridin-2-yl]methyl}piperidin-4-yl)oxy]pyrimidine

A mixture of methyl (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-b]pyridine-6-carbimidate (20 mg, 0.03 mmol), acetohydrazide (5.1 mg, 0.07 mmol), DIEA (25 mg, 0.1 mmol) in n-BuOH (1 mL) was stirred at 120° C. for 16 h. After the reaction was completed, the reaction mixture was concentrated and the residue purified by Prep-HPLC to give 2-[(4-chlorophenyl)methyl]-5-fluoro-4-[(1-{[6-(5-methyl-4H-1,2,4-triazol-3-yl)-3-{[(2S)-oxetan-2-yl]methyl}-3H-imidazo[4,5-b]pyridin-2-yl]methyl}piperidin-4-yl)oxy]pyrimidine Compound 162a (3 mg, yield: 15%) as a white solid.

MS Calcd.: 603.2; MS Found: 604.2 [M+H]⁺.

¹H NMR (400 MHz, DMSO-d₆): δ 8.95 (d, J=1.6 Hz, 1H), 8.50 (d, J=3.2 Hz, 1H), 8.46 (s, 1H), 7.30-7.40 (m, 4H), 5.05-5.21 (m, 2H), 4.77-4.85 (m, 1H), 4.65-4.72 (m, 1H), 4.46-4.53 (m, 1H), 4.34-4.40 (m, 1H), 4.09 (s, 2H), 3.89-3.98 (m, 2H), 2.65-2.83 (m, 5H), 2.38-2.46 (m, 4H), 1.93-2.04 (m, 2H), 1.69-1.80 (m, 2H). ¹⁹F-NMR (377 MHz): −157.5.

### Example 42: 2-[(4-chlorophenyl)methyl]-5-fluoro-4-[(1-{[6-(5-methyl-4H-1,2,4-triazol-3-yl)-3-{[(2S)-oxetan-2-yl]methyl}-3H-imidazo[4,5-c]pyridin-2-yl]methyl}piperidin-4-yl)oxy]pyrimidine (Compound 163a)

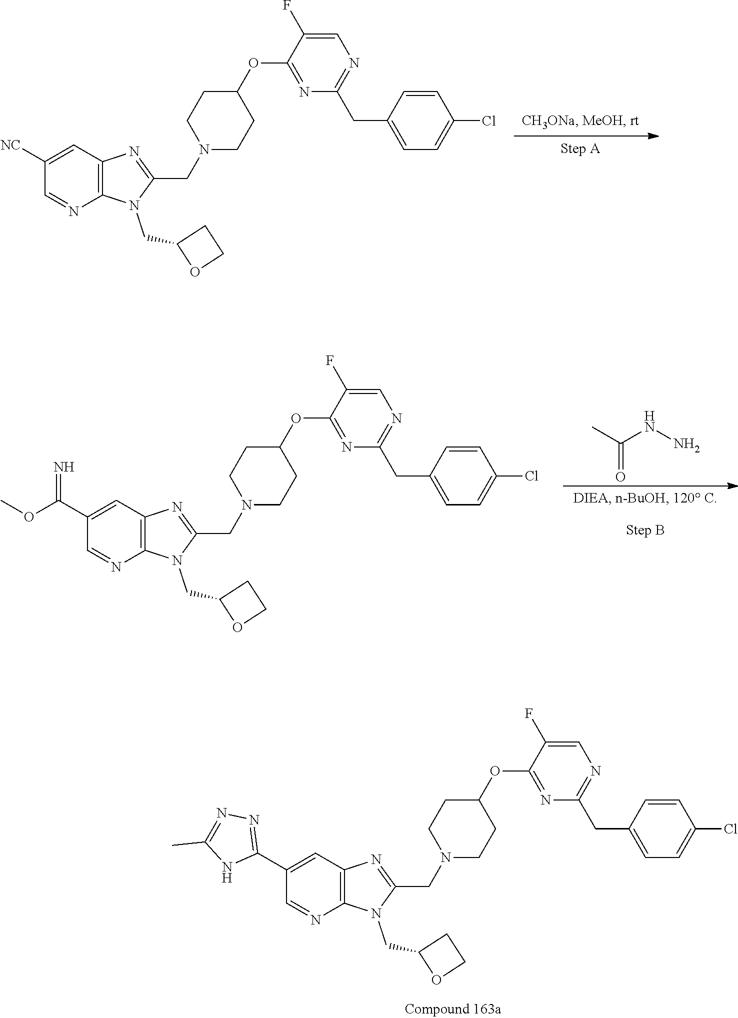

### Step A: methyl (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine-6-carbimidate

A mixture of (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine-6-carbonitrile (40 mg, 0.07 mmol), MeONa (39 mg, 0.73 mmol) in MeOH (2 mL) was stirred at room temperature for 3 h. After the reaction was completed, the mixture was concentrated in vacuum. The residue was purified by Prep-TLC (MeOH/DCM=1/20) to afford methyl (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine-6-carbimidate (39 mg, yield: 92%) as yellow oil. MS Calcd.: 579.2; MS Found: 580.2 [M+H]+.

### Step B: 2-[(4-chlorophenyl)methyl]-5-fluoro-4-[(1-{[6-(5-methyl-4H-1,2,4-triazol-3-yl)-3-{[(2S)-oxetan-2-yl]methyl}-3H-imidazo[4,5-c]pyridin-2-yl]methyl}piperidin-4-yl)oxy]pyrimidine

To a solution of (S)-2-((4-((2-(4-chlorobenzyl)-5-fluoropyrimidin-4-yl)oxy)piperidin-1-yl)methyl)-3-(oxetan-2-ylmethyl)-3H-imidazo[4,5-c]pyridine-6-carbimidate (39 mg, 0.07 mmol), acetohydrazide (9.9 mg, 0.13 mmol), DIEA (26 mg, 0.20 mmol) in n-BuOH (1 mL) was stirred at 120° C. for 5 h. After the reaction was completed, the reaction mixture was concentrated and purified by Prep-HPLC to give 2-[(4-chlorophenyl)methyl]-5-fluoro-4-[(1-{[6-(5-methyl-4H-1,2,4-triazol-3-yl)-3-{[(2S)-oxetan-2-yl]methyl}-3H-imidazo[4,5-c]pyridin-2-yl]methyl}piperidin-4-yl)oxy]pyrimidine Compound 163a (8 mg, yield: 22%) as a white solid.

MS Calcd.: 603.2; MS Found: 604.2 [M+H]⁺.

¹H NMR (400 MHz, DMSO-d₆): δ 9.21 (s, 1H), 8.56 (d, J=3.2 Hz, 1H), 8.31 (s, 1H), 7.30-7.38 (m, 4H), 5.25-5.40 (m, 1H), 5.05-5.13 (m, 1H), 4.70-4.93 (m, 3H), 4.48-4.55 (m, 1H), 4.33-4.43 (m, 1H), 4.10 (s, 2H), 3.01-3.11 (m, 2H), 2.63-2.82 (m, 2H), 2.32-2.45 (m, 5H), 1.95-2.30 (m, 5H). ¹⁹F-NMR (377 MHz): −156.97.

### Example 43: 2-{[4-({2-[(4-chlorophenyl)methyl]pyrimidin-4-yl}oxy)piperidin-1-yl]methyl}-5-(6-methylpyridazin-3-yl)-1-{[(2S)-oxetan-2-yl]methyl}-1H-1,3-benzodiazole (Compound 124a)

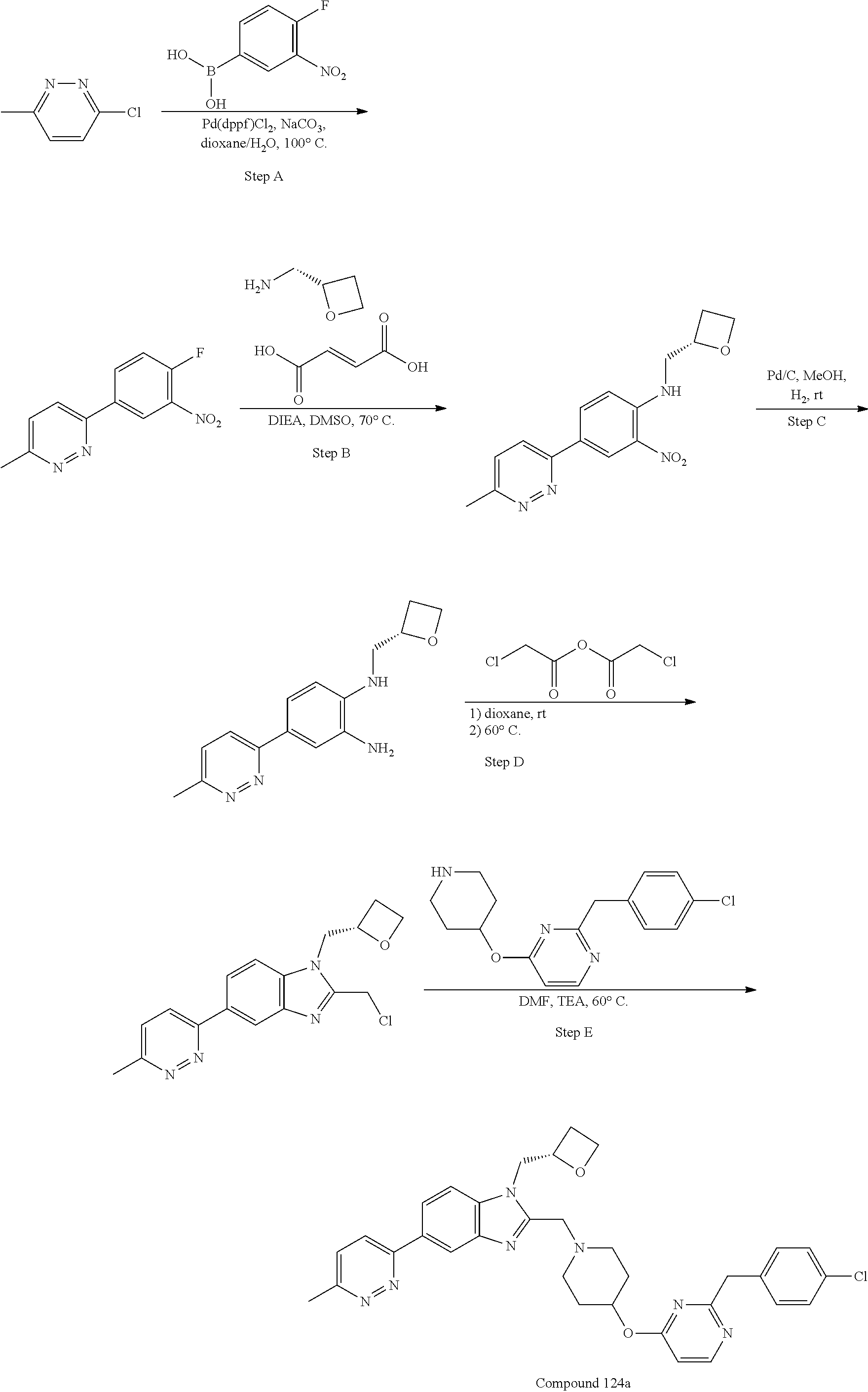

### Step A: 3-(4-fluoro-3-nitrophenyl)-6-methylpyridazine

To a solution of 3-chloro-6-methylpyridazine (600 mg, 4.7 mmol) and (4-fluoro-3-nitrophenyl)boronic acid (1.3 g, 7.0 mmol) in dioxane/H₂O (20 mL/5 mL) was added Na₂CO₃ (1.5 g, 14.0 mmol) and Pd(dppf)Cl₂ (341 mg, 0.47 mmol). The mixture was stirred at 100° C. for 16 hours under N₂. After the reaction was completed, the mixture was filtered. The filtrate was concentrated, dissolved in EA (50 mL), washed with brine (20 mL×3). The organic layer was dried over anhydrous sodium sulfate, filtered, and evaporated to dryness. The organic layer was purified by column chromatography on silica gel (PE/EA=1/1) to give 3-(4-fluoro-3-nitrophenyl)-6-methylpyridazine (820 mg, 75% yield) as an orange solid. MS Calcd.: 233.1; MS Found: 234.1 [M+H]⁺.

### Step B: (S)-2-nitro-N-(oxetan-2-ylmethyl)-4-(pyridazin-3-yl)aniline

A mixture of 3-(4-fluoro-3-nitrophenyl)-6-methylpyridazine (400 mg, 1.7 mmol), (S)-oxetan-2-ylmethanamine fumarate (299 mg, 2.1 mmol) and DIEA (664 mg, 5.2 mmol) in DMSO (6 mL) was stirred at 70° C. for 2 hours. After the reaction was completed, the mixture was diluted with EA (50 mL), washed with brine (20 mL×3). The organic layer was dried over anhydrous sodium sulfate, filtered, and evaporated to dryness. The residue was purified by column chromatography on silica gel (DCM/MeOH=10/1) to give (S)-2-nitro-N-(oxetan-2-ylmethyl)-4-(pyridazin-3-yl)aniline (450 mg, 87% yield) as an orange solid. MS Calcd.: 300.1; MS Found: 301.1 [M+H]⁺.

### Step C: (S)-4-(6-methylpyridazin-3-yl)-N1-(oxetan-2-ylmethyl)benzene-1,2-diamine

A mixture of (S)-2-nitro-N-(oxetan-2-ylmethyl)-4-(pyridazin-3-yl)aniline (450 mg, 1.5 mmol) and Pd/C (200 mg, 10% on carbon, wetted with ca. 55% water) in MeOH (5 mL) was stirred at room temperature under H₂ (1 atm) for 2 hours. After the reaction was completed, the mixture was filtered and the filtrate evaporated to dryness to give (S)-4-(6-methylpyridazin-3-yl)-N1-(oxetan-2-ylmethyl)benzene-1,2-diamine (370 mg) as a dark brown solid. MS Calcd.: 270.1; MS Found: 271.3 [M+H]⁻.

### Step D: (S)-2-(chloromethyl)-5-(6-methylpyridazin-3-yl)-1-(oxetan-2-ylmethyl)-1H-benzo[d]imidazole

To a mixture of (S)-4-(6-methylpyridazin-3-yl)-N1-(oxetan-2-ylmethyl)benzene-1,2-diamine (200 mg) in dioxane (5 mL) was added chloroacetic anhydride (152 mg, 0.89 mmol). The resulting mixture was stirred at room temperature for 2 hours, then heated to 60° C. for 16 hours. After the reaction was completed, the mixture was diluted with EA (50 mL), washed with sat NaHCO₃ (10 mL×1) and H₂O (10 mL×2). The organic layer was dried over anhydrous sodium sulfate, filtered and evaporated to dryness. The residue was purified by column chromatography on silica gel (DCM/MeOH=10/1) to give (S)-2-(chloromethyl)-5-(6-methylpyridazin-3-yl)-1-(oxetan-2-ylmethyl)-1H-benzo[d]imidazole (70 mg, 29% yield) as a yellow solid. MS Calcd.: 328.1; MS Found: 329.1 [M+H]⁺.

### Step E: 2-{[4-({2-[(4-chlorophenyl)methyl]pyrimidin-4-yl}oxy)piperidin-1-yl]methyl}-5-(6-methylpyridazin-3-yl)-1-{[(2S)-oxetan-2-yl]methyl}-1H-1,3-benzodiazole (Compound 124a)

A mixture of (S)-2-(chloromethyl)-5-(6-methylpyridazin-3-yl)-1-(oxetan-2-ylmethyl)-1H-benzo[d]imidazole (70 mg, 0.21 mmol), 2-(4-chlorobenzyl)-4-(piperidin-4-yloxy)pyrimidine (65 mg, 0.21 mmol) and TEA (65 mg, 0.64 mmol) in DMF (2 mL) was stirred at 60° C. for 2 hours. After the reaction was completed, the reaction mixture was purified directly by Prep-HPLC to give 2-{[4-({2-[(4-chlorophenyl)methyl]pyrimidin-4-yl}oxy)piperidin-1-yl]methyl}-5-(6-methylpyridazin-3-yl)-1-{[(2S)-oxetan-2-yl]methyl}-1H-1,3-benzodiazole Compound 124a (18 mg, yield: 14%).

MS Calcd.: 595.2; MS Found: 596.2 [M+H]⁺.

¹H NMR (400 MHz, DMSO-d₆): δ 8.41 (d, J=5.6 Hz, 1H), 8.33 (d, J=1.2 Hz, 1H), 8.17 (d, J=8.4 Hz, 1H), 8.06 (dd, J=8.8, 1.6 Hz, 1H), 7.78 (d, J=8.4 Hz, 1H), 7.63 (d, J=8.8 Hz, 1H), 7.30-7.38 (m, 4H), 6.73 (d, J=6.0 Hz, 1H), 4.98-5.18 (m, 2H), 4.70-4.80 (m, 1H), 4.58-4.67 (m, 1H), 4.47-4.55 (m, 1H), 4.37-4.44 (m, 1H), 4.09 (s, 2H), 3.94 (d, J=13.6 Hz, 1H), 3.82 (d, J=14.0 Hz, 1H), 2.62-2.82 (m, 3H), 2.66 (s, 3H), 2.30-2.52 (m, 3H), 1.90-2.00 (m, 2H), 1.58-1.70 (m, 2H).

### Example 44: 2-{[4-({2-[(4-chlorophenyl)methyl]pyrimidin-4-yl}oxy)piperidin-1-yl]methyl}-5-(3-methyl-1H-1,2,4-triazol-5-yl)-1-{[(2S)-oxetan-2-yl]methyl}-1H-1,3-benzodiazole (Compound 126a)

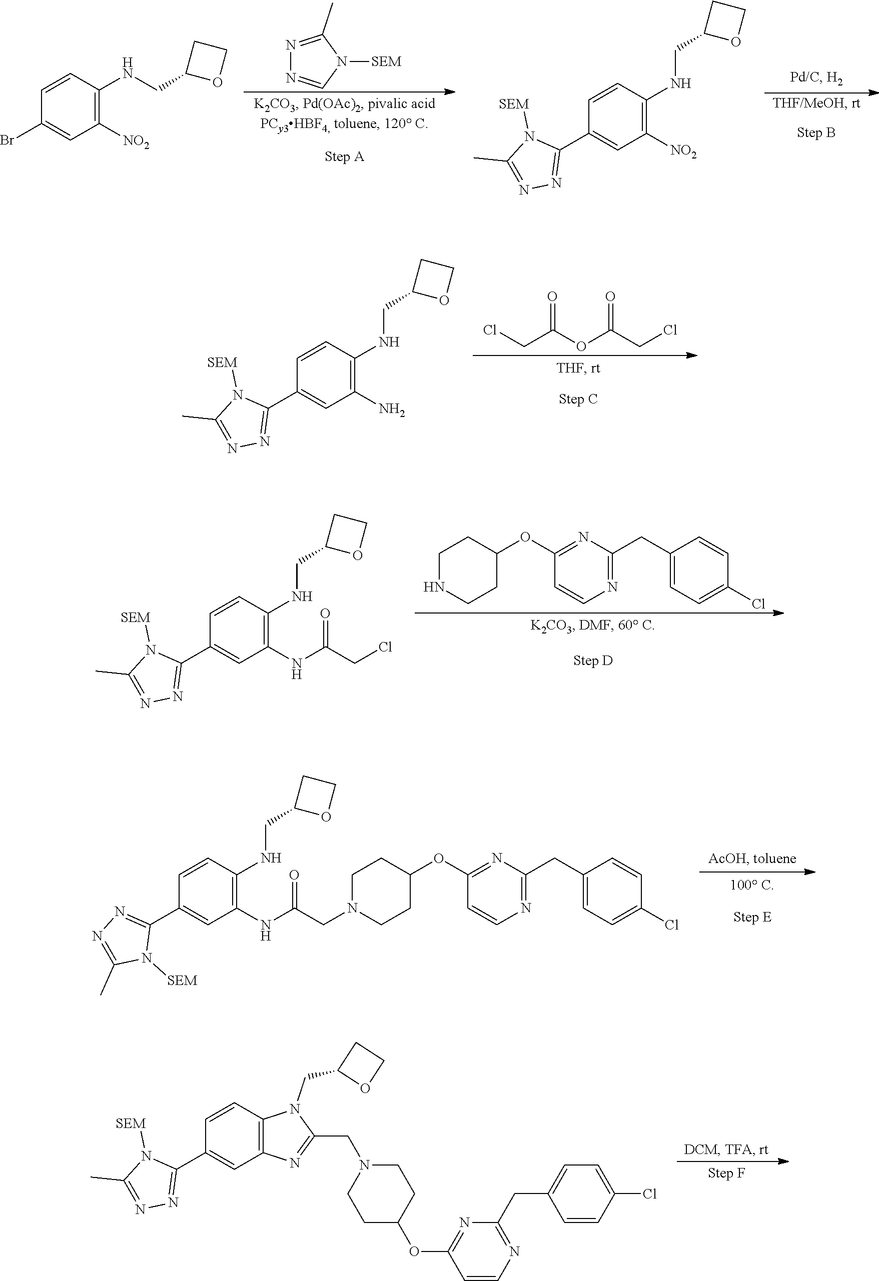

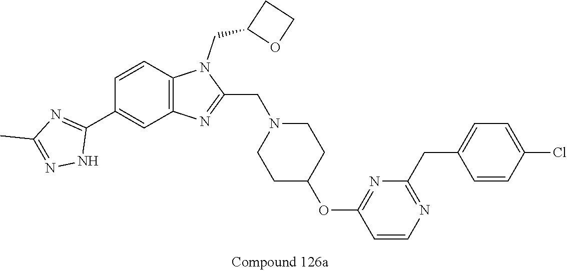 [SMILES: Cc1n[nH]c(-c2ccc3c(c2)nc(CN2CCC(Oc4ccnc(Cc5ccc(Cl)cc5)n4)CC2)n3C[C@@H]2CCO2)n1]

### Step A: (S)-4-(5-methyl-4-((2-(trimethylsilyl)ethoxy)methyl)-4H-1,2,4-triazol-3-yl)-2-nitro-N-(oxetan-2-ylmethyl)aniline

A mixture of (S)-4-bromo-2-nitro-N-(oxetan-2-ylmethyl)aniline (1.0 g, 3.5 mmol), 3-methyl-4-((2-(trimethylsilyl)ethoxy)methyl)-4H-1,2,4-triazole (2.2 g, 10.5 mmol), K₂CO₃ (1.45 mg, 10.5 mmol), Pd(OAc)₂ (79 mg, 0.35 mmol), pivalic acid (236 mg, 2.3 mmol) and PCy₃·HBF₄ (258 mg, 0.70 mmol) in Toluene (12 mL) was stirred at 120° C. for 16 hours. After the reaction was completed, the mixture was filtered, and the filtrate evaporated to dryness. The residue was purified by column chromatography on silica gel (PE/EA=1/2) to give (S)-4-(5-methyl-4-((2-(trimethylsilyl)ethoxy)methyl)-4H-1,2,4-triazol-3-yl)-2-nitro-N-(oxetan-2-ylmethyl)aniline (800 mg, 55% yield) as yellow oil. MS Calcd.: 419.2; MS Found: 420.2 [M+H]⁺.

Note: 3-methyl-4-((2-(trimethylsilyl)ethoxy)methyl)-4H-1,2,4-triazole was prepared by reacting 3-methyl-4H-1,2,4-triazole with SEM-Cl in presence of NaH, the product might be a mixture of the following three structures. We choose b as the representative for subsequent reaction:

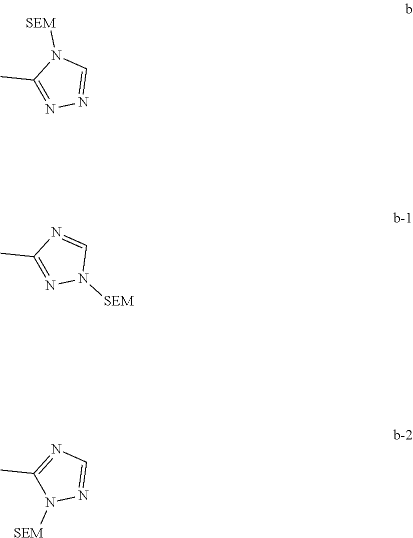

### Step B: (S)-4-(5-methyl-4-((2-(trimethylsilyl)ethoxy)methyl)-4H-1,2,4-triazol-3-yl)-N1-(oxetan-2-ylmethyl)benzene-1,2-diamine

To a solution of (S)-4-(5-methyl-4-((2-(trimethylsilyl)ethoxy)methyl)-4H-1,2,4-triazol-3-yl)-2-nitro-N-(oxetan-2-ylmethyl)aniline (800 mg, 1.9 mmol) and Pd/C (80 mg, 10% on carbon, wetted with ca. 55% water) in THF/MeOH (5.0 mL/5.0 mL) was stirred at rt under H₂ (1 atm) for 2 hours. After the reaction was completed, the mixture was filtered and the filtrate evaporated to dryness to give (S)-4-(5-methyl-4-((2-(trimethylsilyl)ethoxy)methyl)-4H-1,2,4-triazol-3-yl)-N1-(oxetan-2-ylmethyl)benzene-1,2-diamine (700 mg, 94% yield) as yellow oil. MS Calcd.: 389.2; MS Found: 390.1 [M+H]⁻.

### Step C: (S)-2-chloro-N-(5-(5-methyl-4-((2-(trimethylsilyl)ethoxy)methyl)-4H-1,2,4-triazol-3-yl)-2-((oxetan-2-ylmethyl)amino)phenyl)acetamide

To a mixture of (S)-4-(5-methyl-4-((2-(trimethylsilyl)ethoxy)methyl)-4H-1,2,4-triazol-3-yl)-N1-(oxetan-2-ylmethyl)benzene-1,2-diamine (300 mg) in THE (6.0 mL) was added chloroacetic anhydride (132 mg, 0.8 mmol). The resulting mixture was stirred at rt for 2 hours. After the reaction was completed, the mixture was diluted with EA (30.0 mL), washed with H₂O (20.0 mL×2). The organic layer was dried over anhydrous sodium sulfate, filtered, and evaporated to dryness. The residue was purified by column chromatography on silica gel (PE/EA=1/2) to give (S)-2-chloro-N-(5-(5-methyl-4-((2-(trimethylsilyl)ethoxy)methyl)-4H-1,2,4-triazol-3-yl)-2-((oxetan-2-ylmethyl)amino)phenyl)acetamide (60 mg, 17% yield) as yellow oil. MS Calcd.: 465.2; MS Found: 466.1 [M+H]⁺.

### Step D: (S)-2-(4-((2-(4-chlorobenzyl)pyrimidin-4-yl)oxy)piperidin-1-yl)-N-(5-(5-methyl-4-((2-(trimethylsilyl)ethoxy)methyl)-4H-1,2,4-triazol-3-yl)-2-((oxetan-2-ylmethyl)amino)phenyl) acetamide

A mixture of (S)-2-chloro-N-(5-(5-methyl-4-((2-(trimethylsilyl)ethoxy)methyl)-4H-1,2,4-triazol-3-yl)-2-((oxetan-2-ylmethyl)amino)phenyl)acetamide (60 mg, 0.11 mmol), 2-(4-chlorobenzyl)-4-(piperidin-4-yloxy)pyrimidine (39 mg, 0.13 mmol) and K₂CO₃ (53 mg, 0.39 mmol) in DMF (2.0 mL) was stirred at 60° C. for 1 hour. After the reaction was completed, the mixture was filtered and the filtrate evaporated to dryness, The residue was purified by Prep-TLC (DCM/MeOH=20/1) to give (S)-2-(4-((2-(4-chlorobenzyl)pyrimidin-4-yl)oxy)piperidin-1-yl)-N-(5-(5-methyl-4-((2-(trimethylsilyl)ethoxy)methyl)-4H-1,2,4-triazol-3-yl)-2-((oxetan-2-ylmethyl)amino)phenyl)acetamide (70 mg, yield: 74%). MS Calcd.: 732.3; MS Found: 733.2 [M+H]⁺.

### Step E: (S)-2-((4-((2-(4-chlorobenzyl)pyrimidin-4-yl)oxy)piperidin-1-yl)methyl)-5-(5-methyl-4-((2-(trimethylsilyl)ethoxy)methyl)-4H-1,2,4-triazol-3-yl)-1-(oxetan-2-ylmethyl)-1H-benzo[d]imidazole

A mixture of (S)-2-(4-((2-(4-chlorobenzyl)pyrimidin-4-yl)oxy)piperidin-1-yl)-N-(5-(5-methyl-4-((2-(trimethylsilyl)ethoxy)methyl)-4H-1,2,4-triazol-3-yl)-2-((oxetan-2-ylmethyl)amino)phenyl)acetamide (70 mg, 0.1 mmol) in Toluene/AcOH (4.0 mL/0.3 mL) was stirred at 100° C. for 16 hours. After the reaction was completed, the mixture was diluted with EA (20 mL) and washed with Sat. NaHCO₃ (5 mL). The organic layer was washed with brine (10 mL), then dried over anhydrous sodium sulfate, filtered and evaporated to dryness. The residue was purified by Prep-TLC (DCM/MeOH=25/1) to give (S)-2-((4-((2-(4-chlorobenzyl)pyrimidin-4-yl)oxy)piperidin-1-yl)methyl)-5-(5-methyl-4-((2-(trimethylsilyl)ethoxy)methyl)-4H-1,2,4-triazol-3-yl)-1-(oxetan-2-ylmethyl)-1H-benzo[d]imidazole (40 mg, yield: 59%) as yellow oil. MS Calcd.: 714.3; MS Found: 715.2 [M+H]⁺.

### Step F: 2-{[4-({2-[(4-chlorophenyl)methyl]pyrimidin-4-yl}oxy)piperidin-1-yl]methyl}-5-(3-methyl-1H-1,2,4-triazol-5-yl)-1-{[(2S)-oxetan-2-yl]methyl}-1H-1,3-benzodiazole (Compound 126a)

A mixture of (S)-2-((4-((2-(4-chlorobenzyl)pyrimidin-4-yl)oxy)piperidin-1-yl)methyl)-5-(5-methyl-4-((2-(trimethylsilyl)ethoxy)methyl)-4H-1,2,4-triazol-3-yl)-1-(oxetan-2-ylmethyl)-1H-benzo[d]imidazole (25 mg, 0.035 mmol) and TFA (12 mg, 0.11 mmol) in DCM (2.0 mL) was stirred at rt for 2 hours. After the reaction was completed, the mixture was diluted with EA (20.0 mL) and washed with Sat. NaHCO₃ (10.0 mL). The organic layer was dried over anhydrous sodium sulfate, filtered, and evaporated to dryness. The residue was purified by Prep-HPLC to give 2-{[4-({2-[(4-chlorophenyl)methyl]pyrimidin-4-yl}oxy)piperidin-1-yl]methyl}-5-(3-methyl-1H-1,2,4-triazol-5-yl)-1-{[(2S)-oxetan-2-yl]methyl}-1H-1,3-benzodiazole Compound 126a (2 mg, yield: 11%). MS Calcd.: 584.2; MS Found: 585.3 [M+H]+

¹H NMR (400 MHz, CD₃OD): δ 8.41 (d, J=6.0 Hz, 1H), 8.36 (d, J=0.8 Hz, 1H), 8.05 (dd, J=8.8 Hz, 1.2 Hz, 1H), 7.74 (d, J=8.4 Hz, 1H), 7.26-7.33 (m, 4H), 6.76 (d, J=6.0 Hz, 1H), 5.33-5.40 (m, 1H), 5.19-5.27 (m, 1H), 4.60-4.75 (m, 5H), 4.40-4.47 (m, 1H), 4.13 (s, 2H), 3.33-3.60 (m, 4H), 2.75-2.85 (m, 1H), 2.45-2.57 (m, 4H), 2.00-2.33 (m, 4H).

### Example 45: 2-{[4-({2-[(4-chlorophenyl)methyl]pyrimidin-4-yl}oxy)piperidin-1-yl]methyl}-5-(2-methyl-1,3-thiazol-5-yl)-1-{[(2S)-oxetan-2-yl]methyl}-1H-1,3-benzodiazole (Compound 129a)

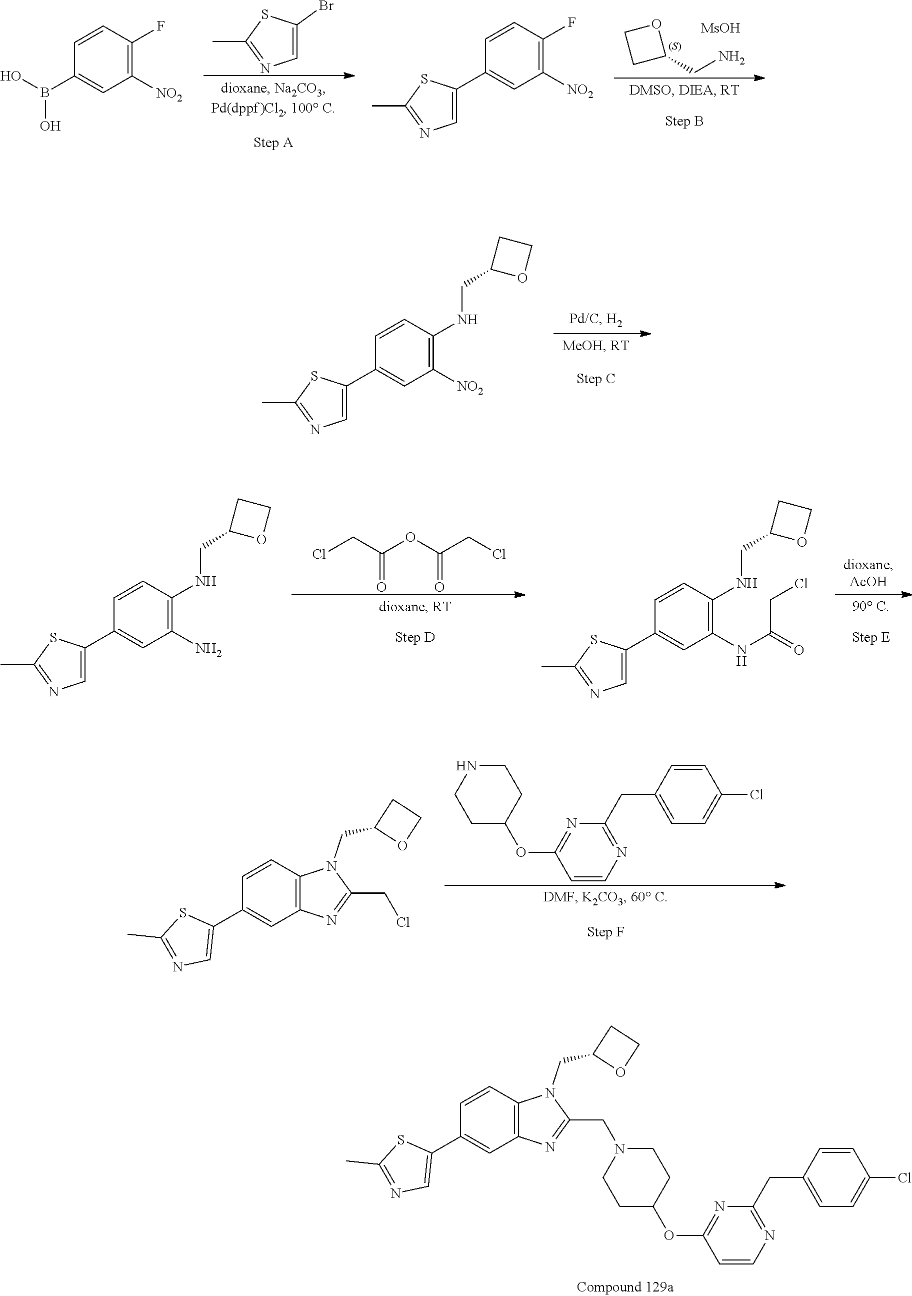

### Step A: 5-(4-fluoro-3-nitrophenyl)-2-methylthiazole

A mixture of (4-fluoro-3-nitrophenyl)boronic acid (472 mg, 2.5 mmol), 5-bromo-2-methylthiazole (300 mg, 1.7 mmol), Pd(dppf)Cl₂ (155 mg, 0.17 mmol) and Na₂CO₃ (538 mg, 5.1 mmol) in dioxane (8 mL) and water (2 mL) was stirred at 100° C. for 5 hours. After the reaction was completed, the mixture was diluted with DCM (50 ml), washed with H₂O (20 ml×3). The organic layer was dried over anhydrous sodium sulfate, filtered, and evaporated to dryness. The residue was purified by column chromatography on silica gel (PE/EA=4/1) to give 5-(4-fluoro-3-nitrophenyl)-2-methylthiazole (350 mg, 87% yield) as a yellow solid. MS Calcd.: 238.0; MS Found: 239.0 [M+H]⁺.

### Step B: (S)-4-(2-methylthiazol-5-yl)-2-nitro-N-(oxetan-2-ylmethyl)aniline

To a solution of 5-(4-fluoro-3-nitrophenyl)-2-methylthiazole (350 mg, 1.5 mmol), (S)-oxetan-2-ylmethanamine MsOH salt (322 mg, 1.8 mmol) and DIEA (581 mg, 4.5 mmol) in DMSO (6 mL) was stirred at room temperature for 3 hours. After the reaction was completed, the mixture was diluted with DCM (50 mL), washed with H₂O (20 mL×3). The organic layer was dried over anhydrous sodium sulfate, filtered, and evaporated to dryness. The residue was purified by column chromatography on silica gel (PE/EA=4/1) to give (S)-4-(2-methylthiazol-5-yl)-2-nitro-N-(oxetan-2-ylmethyl)aniline (400 mg, 89% yield) as a yellow solid. MS Calcd.: 305.1; MS Found: 306.0 [M+H]⁺.

### Step C: (S)-4-(2-methylthiazol-5-yl)-N1-(oxetan-2-ylmethyl)benzene-1,2-diamine

To a solution of (S)-4-(2-methylthiazol-5-yl)-2-nitro-N-(oxetan-2-ylmethyl)aniline (400 mg, 1.3 mmol) and Pd/C (760 mg, 10% on carbon, wetted with ca. 55% water) in MeOH (5 mL) was stirred at rt under H₂ (1 atm) for 1 hour. After the reaction was completed, the mixture was filtered and the filtrate evaporated to dryness to give (S)-4-(2-methylthiazol-5-yl)-N1-(oxetan-2-ylmethyl)benzene-1,2-diamine (300 mg, 83% yield) as a yellow solid. MS Calcd.: 275.1; MS Found: 276.0 [M+H]⁻.

### Step D: (S)-2-chloro-N-(5-(2-methylthiazol-5-yl)-2-((oxetan-2-ylmethyl)amino)phenyl) acetamide

To a mixture of (S)-4-(2-methylthiazol-5-yl)-N1-(oxetan-2-ylmethyl)benzene-1,2-diamine (300 mg) in dioxane (3 mL) was added chloroacetic anhydride (224 mg, 1.3 mmol). The resulting mixture was stirred at R^{T} for 12 hours. After the reaction was completed, the mixture was diluted with EA (50 ml), washed with H₂O (10 ml×3). The organic layer was dried over anhydrous sodium sulfate, filtered, and evaporated to dryness. The residue was purified by column chromatography on silica gel (PE/EA=1/1) to give (S)-2-chloro-N-(5-(2-methylthiazol-5-yl)-2-((oxetan-2-ylmethyl)amino)phenyl)acetamide (200 mg, 52% yield) as a yellow oil. MS Calcd.: 351.1; MS Found: 352.1 [M+H]⁺.

### Step E: (S)-5-(2-(chloromethyl)-1-(oxetan-2-ylmethyl)-1H-benzo[d]imidazol-5-yl)-2-methylthiazole

A mixture of (S)-2-chloro-N-(5-(2-methylthiazol-5-yl)-2-((oxetan-2-ylmethyl)amino)phenyl)acetamide (200 mg, 0.57 mmol) and AcOH (6.6 mg, 0.11 mmol) in dioxane (3 mL) was stirred at 90° C. for 4 hours. After the reaction was completed, the reaction was quenched with Sat. NaHCO₃ (1 mL). The mixture was diluted with EA (50 ml), washed with H₂O (10 ml×3). The organic layer was dried over anhydrous sodium sulfate, filtered, and evaporated to dryness. The residue was purified by column chromatography on silica gel (DCM/MeOH=20/1) to give (S)-5-(2-(chloromethyl)-1-(oxetan-2-ylmethyl)-1H-benzo[d]imidazol-5-yl)-2-methylthiazole (150 mg, yield: 79%) as a yellow solid. MS Calcd.: 333.1; MS Found: 334.0 [M+H]⁺.

### Step F: 2-{[4-({2-[(4-chlorophenyl)methyl]pyrimidin-4-yl}oxy)piperidin-1-yl]methyl}-5-(2-methyl-1,3-thiazol-5-yl)-1-{[(2S)-oxetan-2-yl]methyl}-1H-1,3-benzodiazole (Compound 129a)

A mixture of (S)-5-(2-(chloromethyl)-1-(oxetan-2-ylmethyl)-1H-benzo[d]imidazol-5-yl)-2-methylthiazole (150 mg, 0.45 mmol), 2-(4-chlorobenzyl)-4-(piperidin-4-yloxy)pyrimidine (206 mg, 0.68 mmol) and K₂CO₃ (73 mg, 0.69 mmol) in DMF (2 mL) was stirred at 60° C. for 1 hour. After the reaction was completed, the reaction mixture was purified directly by Prep-HPLC to give 2-{[4-({2-[(4-chlorophenyl)methyl]pyrimidin-4-yl}oxy)piperidin-1-yl]methyl}-5-(2-methyl-1,3-thiazol-5-yl)-1-{[(2S)-oxetan-2-yl]methyl}-1H-1,3-benzodiazole Compound 129a (25 mg, yield: 9%).

MS Calcd.: 600.2; MS Found: 601.3 [M+H]⁺.

¹H NMR (400 MHz, DMSO-d₆): δ 8.41 (d, J=6.0 Hz, 1H), 7.97 (s, 1H), 7.81 (s, 1H), 7.67 (d, J=8.8 Hz, 1H), 7.48 (dd, J=8.6 Hz, 1.4 Hz, 1H), 7.30-7.38 (m, 4H), 6.72 (d, J=5.6 Hz, 1H), 4.97-5.13 (m, 2H), 4.66-4.75 (m, 1H), 4.53-4.62 (m, 1H), 4.45-4.53 (m, 1H), 4.33-4.40 (m, 1H), 4.08 (s, 2H), 3.91 (d, J=19.6 Hz, 1H), 3.78 (d, J=13.6 Hz, 1H), 2.63-2.82 (m, 5H), 2.30-2.48 (m, 3H), 1.88-1.97 (m, 2H), 1.55-1.67 (m, 2H).

### Example 46: 2-{[4-({2-[(4-chlorophenyl)methyl]pyrimidin-4-yl}oxy)piperidin-1-yl]methyl}-5-(1-methyl-1H-1,2,3-triazol-4-yl)-1-{[(2S)-oxetan-2-yl]methyl}-1H-1,3-benzodiazole (Compound 130a)

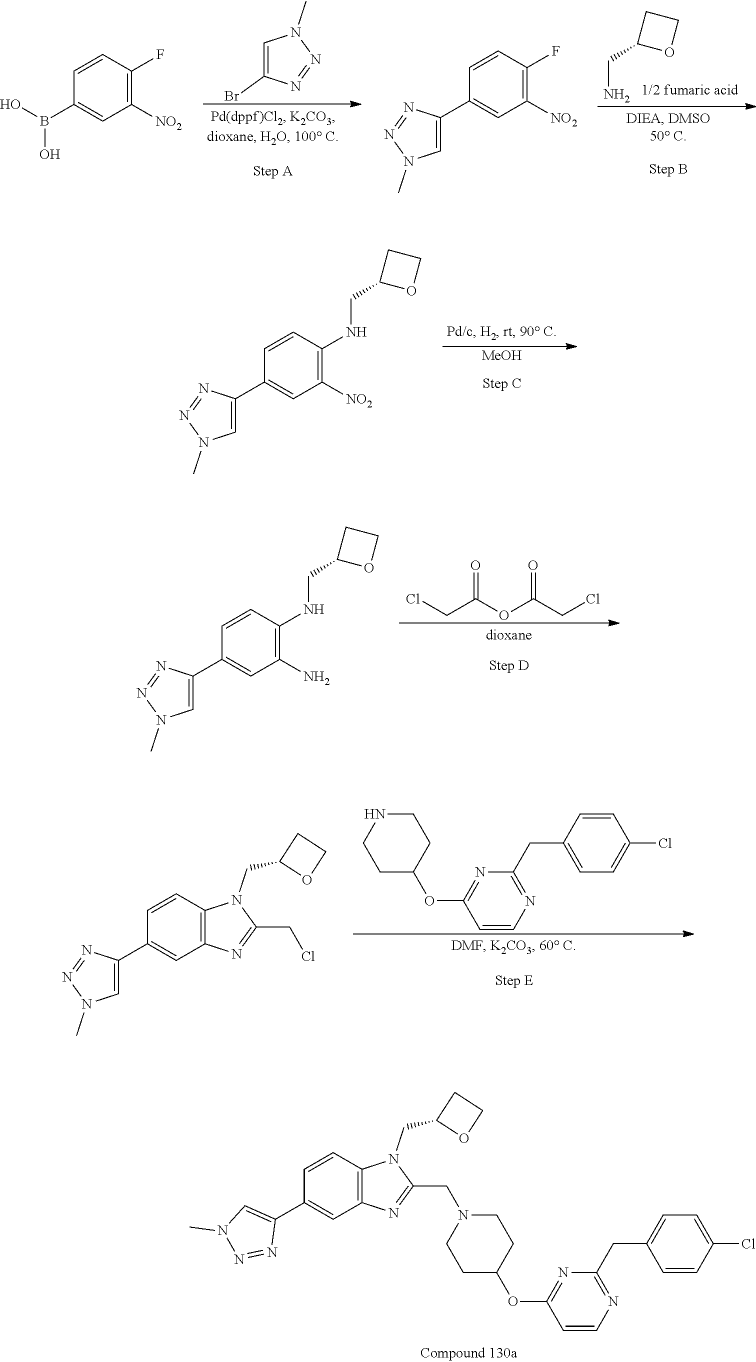

### Step A: 4-(4-fluoro-3-nitrophenyl)-1-methyl-1H-1,2,3-triazole

A mixture of 4-bromo-1-methyl-1H-1,2,3-triazole (300 mg, 1.89 mmol), (4-fluoro-3-nitrophenyl)boronic acid (379 mg, 2.0 mmol), Pd(dppf)Cl₂ (130 mg, 0.19 mmol) and K₂CO₃ (770 mg, 5.6 mmol) in dioxane (10 mL) and water (1 mL) was stirred at 100° C. for 16 hours under N₂. After the reaction was completed, the mixture was diluted with EA (70 mL), washed with H₂O (40 mL×3). The organic layer was dried over anhydrous sodium sulfate, filtered, and evaporated to dryness. The residue was purified by Prep-TLC (PE/EA=2/1) to give 4-(4-fluoro-3-nitrophenyl)-1-methyl-1H-1,2,3-triazole (280 mg, 67% yield) as a yellow solid. MS Calcd.: 222.1; MS Found: 223.3 [M+H]⁺.

### Step B: (S)-4-(1-methyl-1H-1,2,3-triazol-4-yl)-2-nitro-N-(oxetan-2-ylmethyl)aniline

To a solution of 4-(4-fluoro-3-nitrophenyl)-1-methyl-1H-1,2,3-triazole (260 mg, 1.17 mmol), (S)-oxetan-2-ylmethanamine (0.5 fumaric acid salt) (203 mg, 1.4 mmol) and DIEA (453 mg, 3.5 mmol) in DMSO (5 mL) was stirred at 50° C. for 2 hours. After the reaction was completed, the mixture was diluted with DCM (50 mL), washed with H₂O (20 mL×3). The organic layer was dried over anhydrous sodium sulfate, filtered, and evaporated to dryness. The residue was purified by column chromatography on silica gel (PE/EA=1/1) to give (S)-4-(1-methyl-1H-1,2,3-triazol-4-yl)-2-nitro-N-(oxetan-2-ylmethyl)aniline (270 mg, 79% yield) as a yellow solid. MS Calcd.: 289.1; MS Found: 290.5 [M+H]⁺.

### Step C: (S)-4-(1-methyl-1H-1,2,3-triazol-4-yl)-N1-(oxetan-2-ylmethyl)benzene-1,2-diamine

To a mixture of (S)-4-(1-methyl-1H-1,2,3-triazol-4-yl)-2-nitro-N-(oxetan-2-ylmethyl)aniline (260 mg, 0.9 mmol) and Pd/C (30 mg, 10% on carbon, wetted with ca. 55% water) in MeOH (6 mL) was stirred at rt under H₂ (1 atm) for 90 mins. After the reaction was completed, the mixture was filtered and the filtrate evaporated to dryness to give (S)-4-(1-methyl-1H-1,2,3-triazol-4-yl)-N1-(oxetan-2-ylmethyl)benzene-1,2-diamine (190 mg, 81% yield) as a dark brown solid. MS Calcd.: 259.1; MS Found: 260.1 [M+H]⁻.

### Step D: (S)-2-(chloromethyl)-5-(1-methyl-1H-1,2,3-triazol-4-yl)-1-(oxetan-2-ylmethyl)-1H-benzo[d]imidazole

To a mixture of (S)-4-(1-methyl-1H-1,2,3-triazol-4-yl)-N1-(oxetan-2-ylmethyl)benzene-1,2-diamine (50 mg) in dioxane (4 mL) was added 2-chloroacetic anhydride (40 mg, 0.37 mmol). The resulting mixture was stirred at R^{T} for 5 hours and at 60° C. for 16 h. After the reaction was completed, the mixture was dried under reduced pressure to give (S)-2-(chloromethyl)-5-(1-methyl-1H-1,2,3-triazol-4-yl)-1-(oxetan-2-ylmethyl)-1H-benzo[d]imidazole (50 mg, crude) as a white solid. MS Calcd.: 317.1; MS Found: 318.1 [M+H]⁺.

### Step E: 2-{[4-({2-[(4-chlorophenyl)methyl]pyrimidin-4-yl}oxy)piperidin-1-yl]methyl}-5-(1-methyl-1H-1,2,3-triazol-4-yl)-1-{[(2S)-oxetan-2-yl]methyl}-1H-1,3-benzodiazole (Compound 130a)

A mixture of (S)-2-(chloromethyl)-5-(1-methyl-1H-1,2,3-triazol-4-yl)-1-(oxetan-2-ylmethyl)-1H-benzo[d]imidazole (50 mg, 0.16 mmol), 2-(4-chlorobenzyl)-4-(piperidin-4-yloxy)pyrimidine (57 mg, 0.18 mmol) and K₂CO₃ (62 mg, 0.45 mmol) in DMF (3 mL) was stirred at 60° C. for 3 hours. After the reaction was completed, the reaction mixture was purified directly by Prep-HPLC to give 2-{[4-({2-[(4-chlorophenyl)methyl]pyrimidin-4-yl}oxy)piperidin-1-yl]methyl}-5-(1-methyl-1H-1,2,3-triazol-4-yl)-1-{[(2S)-oxetan-2-yl]methyl}-1H-1,3-benzodiazole Compound 130a (17 mg, yield: 19%) as a yellow solid.

MS Calcd.: 584.2; MS Found: 585.2 [M+H]⁺.

¹H NMR (400 MHz, DMSO-d₆): δ 8.49 (s, 1H), 8.41 (d, J=6.0 Hz, 1H), 8.00-8.04 (m, 1H), 7.66-7.75 (m, 2H), 7.30-7.37 (m, 4H), 6.73 (d, J=5.6 Hz, 1H), 4.98-5.13 (m, 2H), 4.67-4.75 (m, 1H), 4.54-4.62 (m, 1H), 4.47-4.54 (m, 1H), 4.36-4.42 (m, 1H), 4.09 (s, 3H), 4.08 (s, 2H), 3.91 (d, J=13.2 Hz, 1H), 3.78 (d, J=13.6 Hz, 1H), 2.66-2.83 (m, 3H), 2.40-2.50 (m, 1H), 2.30-2.40 (m, 2H), 1.87-1.98 (m, 2H), 1.56-1.69 (m, 2H).

### Example 47: 2-[(4-chlorophenyl)methyl]-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine (Compound 150a)

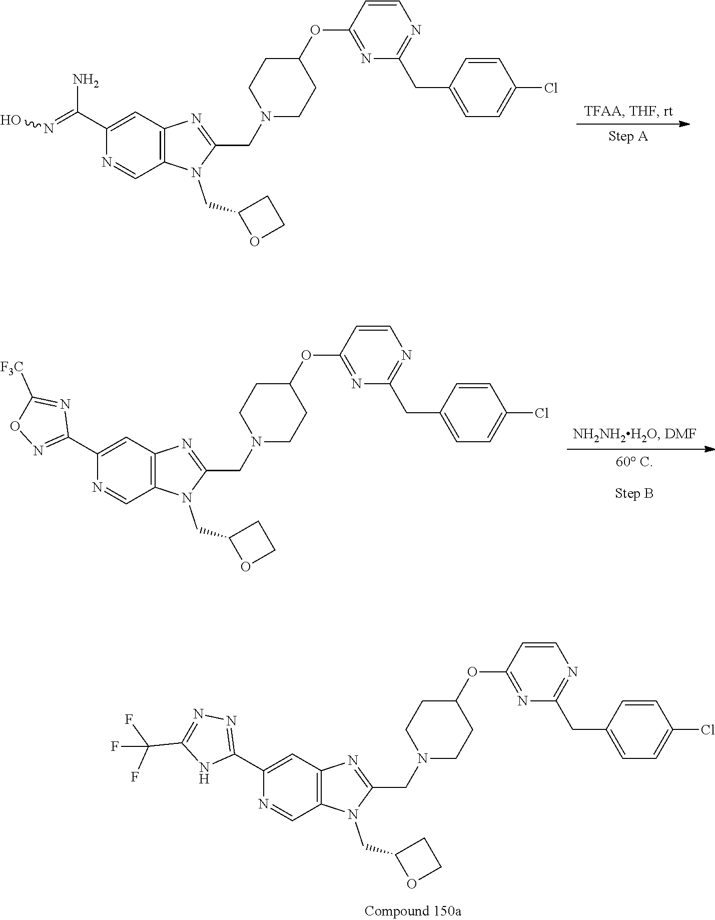 [SMILES: NC(=NO)c1cc2nc(CN3CCC(Oc4ccnc(Cc5ccc(Cl)cc5)n4)CC3)n(C[C@@H]3CCO3)c2cn1 ; FC(F)(F)c1nc(-c2cc3nc(CN4CCC(Oc5ccnc(Cc6ccc(Cl)cc6)n5)CC4)n(C[C@@H]4CCO4)c3cn2)no1 ; FC(F)(F)c1nnc(-c2cc3nc(CN4CCC(Oc5ccnc(Cc6ccc(Cl)cc6)n5)CC4)n(C[C@@H]4CCO4)c3cn2)[nH]1]

### Step A: (S)-3-(2-((4-((2-(4-chlorobenzyl)pyrimidin-4-yl)oxy)piperidin-1-yl)methyl)-3-(oxetan-2-ylmethyl)-3H-imidazo[4,5-c]pyridin-6-yl)-5-(trifluoromethyl)-1,2,4-oxadiazole

A mixture of (S)-2-((4-((2-(4-chlorobenzyl)pyrimidin-4-yl)oxy)piperidin-1-yl)methyl)-N′-hydroxy-3-(oxetan-2-ylmethyl)-3H-imidazo[4,5-c]pyridine-6-carboximidamide (50 mg) and TFAA (37 mg, 0.18 mmol) in THF (2 mL) was stirred at rt for 1 hour. After the reaction was completed, the reaction was quenched with sodium bicarbonate aqueous solution (8 mL), extracted with ethyl acetate (10 mL×2). The organic layers were combined, dried over Na₂SO₄, filtered, and concentrated under vacuum to dryness. The residue was purified by column chromatography on silica gel (DCM/MeOH=50/1) to give (S)-3-(2-((4-((2-(4-chlorobenzyl)pyrimidin-4-yl)oxy)piperidin-1-yl)methyl)-3-(oxetan-2-ylmethyl)-3H-imidazo[4,5-c]pyridin-6-yl)-5-(trifluoromethyl)-1,2,4-oxadiazole (20 mg, yield: 35%) as a white solid. MS Calcd.: 640.2; MS Found: 641.4 [M+H]⁺.

### Step B: 2-[(4-chlorophenyl)methyl]-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine (Compound 150a)

A mixture of (S)-3-(2-((4-((2-(4-chlorobenzyl)pyrimidin-4-yl)oxy)piperidin-1-yl)methyl)-3-(oxetan-2-ylmethyl)-3H-imidazo[4,5-c]pyridin-6-yl)-5-(trifluoromethyl)-1,2,4-oxadiazole (20 mg, 0.03 mmol) and N₂H₄·H₂O (2 mg, 0.04 mmol) in DMF (1.5 mL) was stirred at 60° C. for 1 hour. After the reaction was completed, the mixture was purified directly by Prep-HPLC (0.1% FA) to give 2-[(4-chlorophenyl)methyl]-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine Compound 150a (8 mg, yield: 42%).

MS Calcd.: 639.2; MS Found: 640.2 [M+H]⁺.

¹H NMR (400 MHz, DMSO-d₆): δ 9.17 (s, 1H), 8.41 (d, J=5.6 Hz, 1H), 8.29 (s, 1H), 7.30-7.40 (m, 4H), 6.73 (d, J=5.6 Hz, 1H), 5.10-5.20 (m, 1H), 4.96-5.07 (m, 1H), 4.87-4.95 (m, 1H), 4.70-4.78 (m, 1H), 4.47-4.56 (m, 1H), 4.38-4.46 (m, 1H), 4.08 (s, 2H), 4.01 (d, J=13.2 Hz, 1H), 3.87 (d, J=13.2 Hz, 1H), 2.65-2.85 (m, 3H), 2.30-2.52 (m, 3H), 1.87-1.99 (m, 2H), 1.56-1.70 (m, 2H).

### Example 48: 6-[(4-chloro-2-fluorophenyl)methyl]-3-fluoro-2-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 164a)

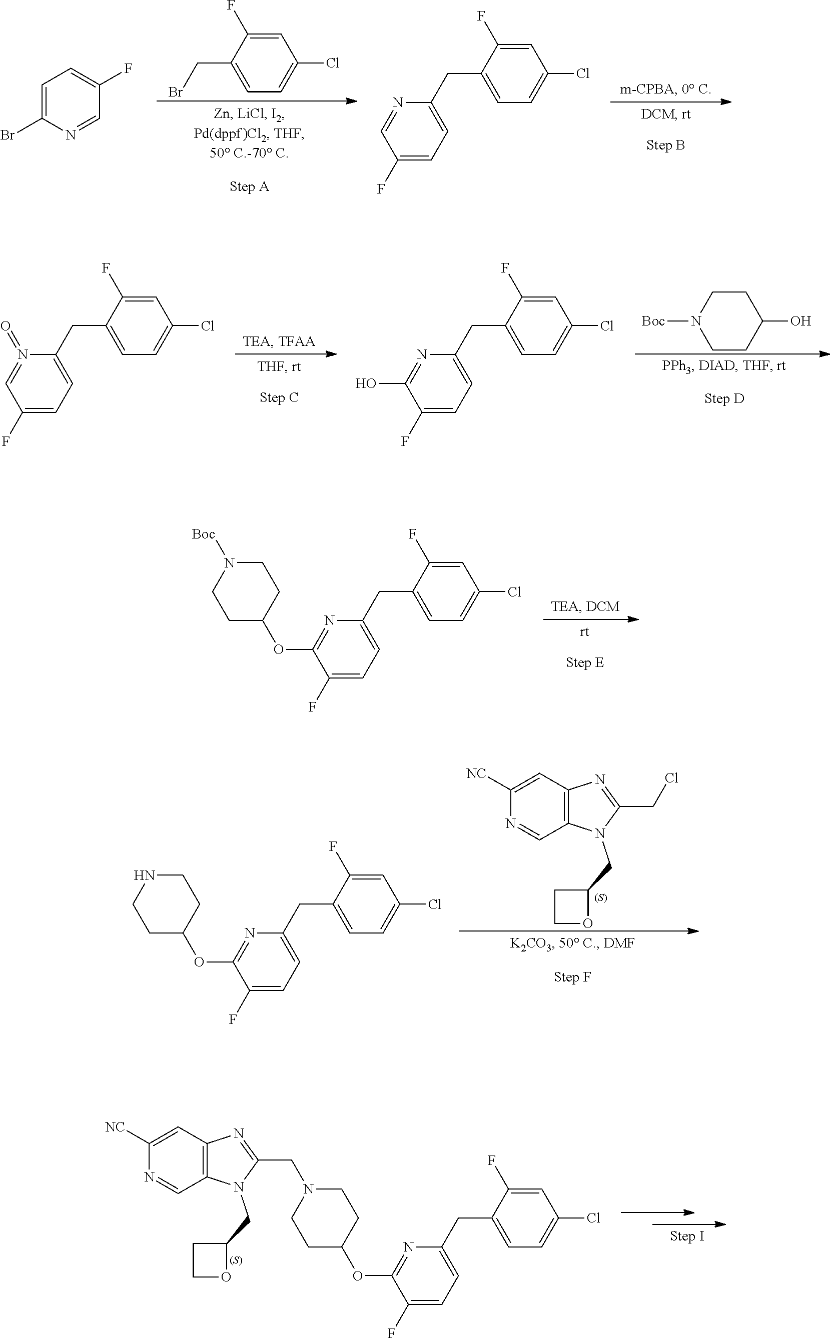

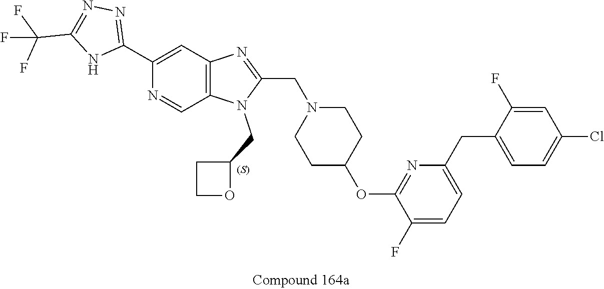

### Step A: 2-(4-chloro-2-fluorobenzyl)-5-fluoropyridine

To a solution of 1-(bromomethyl)-4-chloro-2-fluorobenzene (762 mg, 3.4 mmol) in THE (5 mL) was added Zn (676 mg, 10.3 mmol), LiCl (14 mg, 0.34 mmol) and I₂ (44 mg, 1.7 mmol) under N₂. The reaction mixture was stirred at 50° C. for 0.5 hour. To the mixture was added Pd(dppf)Cl₂ (119 mg, 0.17 mmol), 2-bromo-5-fluoropyridine (300 mg, 1.7 mmol), The reaction mixture was stirred at 70° C. for 2.5 hour. After the reaction was completed, the mixture was filtered and evaporated to dryness. The residue was purified by column chromatography on silica gel (PE/EA=5/1) to give 2-(4-chloro-2-fluorobenzyl)-5-fluoropyridine (310 mg, yield: 75%) as yellow oil. MS Calcd.: 239.0; MS Found: 240.2 [M+H]⁺.

### Step B: 2-(4-chloro-2-fluorobenzyl)-5-fluoropyridine 1-oxide

A mixture of 2-(4-chloro-2-fluorobenzyl)-5-fluoropyridine (310 mg, 1.3 mmol), m-CPBA (557 mg, 3.2 mmol) in DCM (3 mL) at 0° C. The mixture was stirred at room temperature for 16 hours. After the reaction was completed, the mixture was quenched with H₂O (100 mL) and extracted with EA (100 mL*2). The combined organic layer was dried over anhydrous sodium sulfate, filtered, and evaporated to dryness. The residue was purified by column chromatography on silica gel (PE/EA=1/1) to give 2-(4-chloro-2-fluorobenzyl)-5-fluoropyridine 1-oxide (267 mg, yield: 81%) as yellow oil. MS Calcd.: 255.0; MS Found: 256.2 [M+H]⁺.

### Step C: 6-(4-chloro-2-fluorobenzyl)-3-fluoropyridin-2-ol

To a mixture of 2-(4-chloro-2-fluorobenzyl)-5-fluoropyridine 1-oxide (267 mg, 1.0 mmol) in THE (5 mL) was added TFAA (2.2 g, 10.4 mmol) and Et₃N (211 mg, 2.1 mmol). The mixture was stirred at room temperature for 3 hours. After the reaction was completed, the mixture was quenched with H₂O (200 mL) and extracted with DCM (100 mL*3). The extracts were wash with brine (200 mL*3). The organic layer was dried over anhydrous sodium sulfate, filtered, and evaporated to dryness. The residue was purified by column chromatography on silica gel (PE/EA=1/2) to give 6-(4-chloro-2-fluorobenzyl)-3-fluoropyridin-2-ol (130 mg, yield: 48%) as yellow oil. MS Calcd.: 255.0; MS Found: 256.2 [M+H]⁺.

### Step D: tert-butyl 4-((6-(4-chloro-2-fluorobenzyl)-3-fluoropyridin-2-yl)oxy)piperidine-1-carboxylate

To a solution of 6-(4-chloro-2-fluorobenzyl)-3-fluoropyridin-2-ol (130 mg, 0.51 mmol) in THE (5 mL) was added tert-butyl 4-hydroxypiperidine-1-carboxylate (153 mg, 0.76 mmol), PPh₃ (200 mg, 0.76 mmol) and DIAD (154 mg, 0.76 mmol). The mixture was stirred at room temperature for 4 h. After the reaction was completed, the mixture was quenched with H₂O (100 mL) and extracted with EA (100 mL*2). The combined organic layer was dried over anhydrous sodium sulfate, filtered, and evaporated to dryness. The residue was purified by column chromatography on silica gel (PE/EA=1/1) to give tert-butyl 4-((6-(4-chloro-2-fluorobenzyl)-3-fluoropyridin-2-yl)oxy)piperidine-1-carboxylate (120 mg, yield: 53%) as yellow oil. MS Calcd.: 438.1; MS Found: 439.3 [M+H]⁺.

### Step E: 6-(4-chloro-2-fluorobenzyl)-3-fluoro-2-(piperidin-4-yloxy)pyridine

To a mixture of tert-butyl 4-((6-(4-chloro-2-fluorobenzyl)-3-fluoropyridin-2-yl)oxy)piperidine-1-carboxylate (140 mg, 0.27 mmol) in DCM (1.5 mL) was added TFA (0.5 mL). The mixture was stirred at R^{T} for 1 h. After the reaction was completed, the mixture was evaporated to afford 6-(4-chloro-2-fluorobenzyl)-3-fluoro-2-(piperidin-4-yloxy)pyridine (150 mg, crude) as yellow oil. MS Calcd.: 338.1; MS Found: 339.2 [M+H]⁺.

### Step F: (S)-2-((4-((6-(4-chloro-2-fluorobenzyl)-3-fluoropyridin-2-yl)oxy)piperidin-1-yl)methyl)-3-(oxetan-2-ylmethyl)-3H-imidazo[4,5-c]pyridine-6-carbonitrile

A mixture of 6-(4-chloro-2-fluorobenzyl)-3-fluoro-2-(piperidin-4-yloxy)pyridine (150 mg), (S)-2-(chloromethyl)-3-(oxetan-2-ylmethyl)-3H-imidazo[4,5-c]pyridine-6-carbonitrile (60 mg, 0.22 mmol) and K₂CO₃ (500 mg, 3.6 mmol) in DMF (3 mL) was stirred at 50° C. for 1 hours. After the reaction was completed, the mixture was quenched with H₂O (50 mL) and extracted with EA (50 mL*2). The combined organic layer was dried over anhydrous sodium sulfate, filtered, and evaporated to dryness. The residue was purified by column chromatography on silica gel (PE/EA=1/1) to give (S)-2-((4-((6-(4-chloro-2-fluorobenzyl)-3-fluoropyridin-2-yl)oxy)piperidin-1-yl)methyl)-3-(oxetan-2-ylmethyl)-3H-imidazo[4,5-c]pyridine-6-carbonitrile (100 mg, yield: 80%) as yellow oil. MS Calcd.: 564.2; MS Found: 565.3 [M+H]⁺.

### Step I: 6-[(4-chloro-2-fluorophenyl)methyl]-3-fluoro-2-({1-[(3-{1[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 164a)

6-[(4-chloro-2-fluorophenyl)methyl]-3-fluoro-2-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine Compound 164a (15 mg) was obtained with the similar procedure of Compound 149a. MS Calcd.: 674.2; MS Found: 675.1 [M+H]⁺.

¹H NMR (400 MHz, DMSO-d₆): δ 9.16 (s, 1H), 8.28 (s, 1H), 7.53-7.60 (m, 1H), 7.32-7.40 (m, 2H), 7.23 (dd, J=8.4 Hz, 2.0 Hz, 1H), 6.87 (dd, J=8.0 Hz, 2.8 Hz, 1H), 5.11-5.20 (m, 1H), 4.84-4.95 (m, 2H), 4.71-4.78 (m, 1H), 4.38-4.56 (m, 2H), 3.96-4.03 (m, 3H), 3.86 (d, J=13.6 Hz, 1H), 2.65-2.85 (m, 3H), 2.39-2.52 (m, 1H), 2.26-2.37 (m, 2H), 1.84-1.94 (m, 2H), 1.53-1.68 (m, 2H). ¹⁹F-NMR (377 MHz): −63.56, −114.67, −143.61.

### Example 49: 2-{[4-({2-[(4-chloro-2-fluorophenyl)methyl]-5-fluoropyrimidin-4-yl}oxy)piperidin-1-yl]methyl}-1-{[(2S)-oxetan-2-yl]methyl}-1H-1,3-benzodiazole-5-carboxylic acid (Compound 165a)

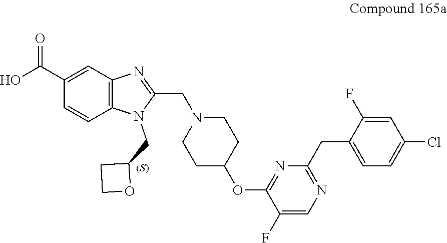

2-{[4-({2-[(4-chloro-2-fluorophenyl)methyl]-5-fluoropyrimidin-4-yl}oxy)piperidin-1-yl]methyl}-1-{[(2S)-oxetan-2-yl]methyl}-1H-1,3-benzodiazole-5-carboxylic acid Compound 165a (19 mg) was obtained with the similar method of Compound 134a as a white solid.

MS Calcd.: 583.2; MS Found: 584.2 [M+H]⁺.

¹H NMR (400 MHz, DMSO-d₆): 8.50 (d, J=2.8 Hz, 1H), 8.17 (s, 1H), 7.85 (d, J=8.4 Hz, 1H), 7.69 (d, J=8.4 Hz, 1H), 7.36-7.44 (m, 2H), 7.25 (d, J=8.8 Hz, 1H), 5.05-5.15 (m, 1H), 4.93-5.04 (m, 1H), 4.70-4.78 (m, 1H), 4.57-4.64 (m, 1H), 4.46-4.54 (m, 1H), 4.34-4.43 (m, 1H), 4.15 (s, 2H), 3.92 (d, J=13.2 Hz, 1H), 3.79 (d, J=13.2 Hz, 1H), 2.65-2.82 (m, 3H), 2.38-2.52 (m, 1H), 2.25-2.38 (m, 2H), 1.87-1.97 (m, 2H), 1.57-1.73 (m, 2H). ¹⁹F-NMR (377 MHz): −114.14, −157.35.

### Example 50: 2-[(4-chloro-2-fluorophenyl)methyl]-4-({1-[(3-{[(2S)-oxetan-2-yl]methyl}-6-(4H-1,2,4-triazol-3-yl)-3H-imidazo[4,5-c]pyridin-2-yl)methyl]piperidin-4-yl}oxy)pyrimidine (Compound 166a)

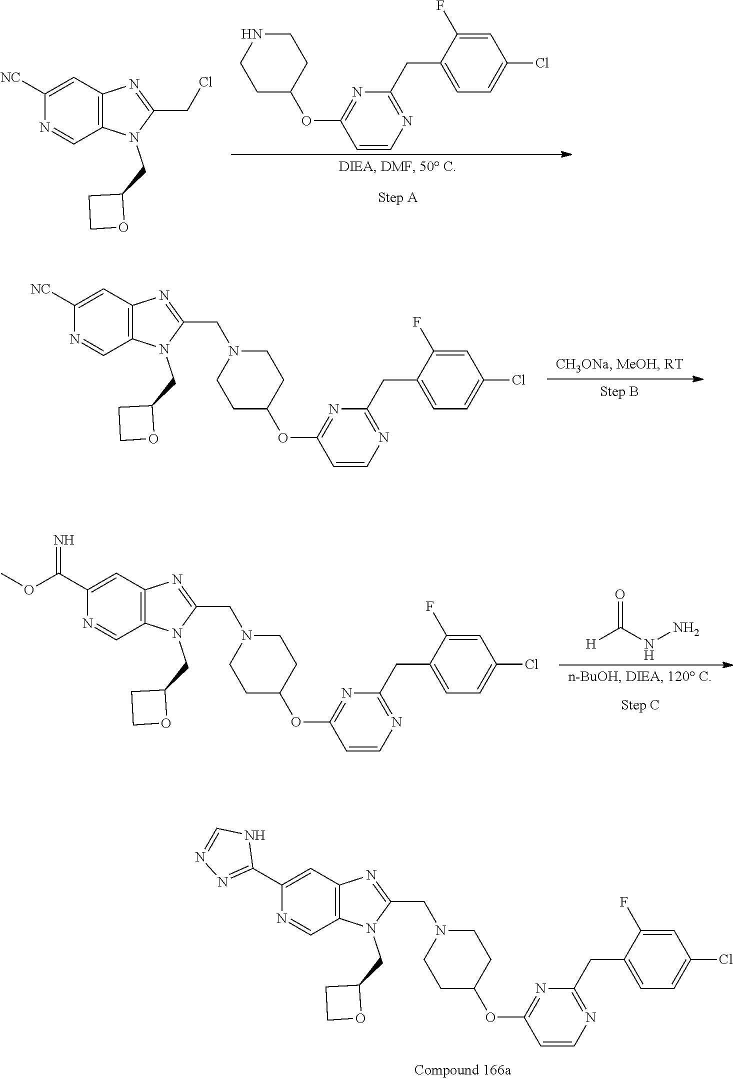

### Step A: (S)-2-((4-((2-(4-chloro-2-fluorobenzyl)pyrimidin-4-yl)oxy)piperidin-1-yl)methyl)-3-(oxetan-2-ylmethyl)-3H-imidazo[4,5-c]pyridine-6-carbonitrile

A mixture of (S)-2-(chloromethyl)-3-(oxetan-2-ylmethyl)-3H-imidazo[4,5-c]pyridine-6-carbonitrile (125 mg, 0.48 mmol), 2-(4-chloro-2-fluorobenzyl)-4-(piperidin-4-yloxy)pyrimidine (190 mg, 0.53 mmol) and DIEA (186 mg, 1.4 mmol) in DMF (5 mL) was stirred at 50° C. for 3 hours. After the reaction was completed, the mixture was diluted with ethyl acetate (30 mL), washed with brine (25 ml×2). The organic layer was dried over sodium sulfate, filtered, and concentrated in vacuum. The residue was purified by column chromatography on silica gel (DCM/MeOH=20/1) to give (S)-2-((4-((2-(4-chloro-2-fluorobenzyl) pyrimidin-4-yl)oxy)piperidin-1-yl)methyl)-3-(oxetan-2-ylmethyl)-3H-imidazo[4,5-c]pyridine-6-carbonitrile (170 mg, yield: 66%) as yellow oil. MS Calcd.: 547.2; MS Found: 548.0 [M+H]⁺.

### Step B: methyl (S)-2-((4-((2-(4-chloro-2-fluorobenzyl)pyrimidin-4-yl)oxy)piperidin-1-yl)methyl)-3-(oxetan-2-ylmethyl)-3H-imidazo[4,5-c]pyridine-6-carbimidate

A mixture of (S)-2-((4-((2-(4-chloro-2-fluorobenzyl)pyrimidin-4-yl)oxy)piperidin-1-yl)methyl)-3-(oxetan-2-ylmethyl)-3H-imidazo[4,5-c]pyridine-6-carbonitrile (100 mg, 0.18 mmol) and CH₃ONa (99 mg, 1.8 mmol) in MeOH (3 mL) was stirred at room temperature for 4 hours. The mixture was poured into cold water and extracted with EtOAc (20 ml×2), washed with water (25 ml×2). The organic layer was dried over sodium sulfate, filtered, and concentrated under reduced pressure. The residue was purified by Prep-TLC (DCM/MeOH=10/1) to give methyl (S)-2-((4-((2-(4-chloro-2-fluorobenzyl)pyrimidin-4-yl)oxy)piperidin-1-yl)methyl)-3-(oxetan-2-ylmethyl)-3H-imidazo[4,5-c]pyridine-6-carbimidate (120 mg, crude, ca.100%) as brown oil. MS Calcd.: 579.2; MS Found: 580.4 [M+H]⁺.

### Step C: 2-[(4-chloro-2-fluorophenyl)methyl]-4-({1-[(3-{[(2S)-oxetan-2-yl]methyl}-6-(4H-1,2,4-triazol-3-yl)-3H-imidazo[4,5-c]pyridin-2-yl)methyl]piperidin-4-yl}oxy)pyrimidine (Compound 166a)

A mixture of methyl (S)-2-((4-((2-(4-chloro-2-fluorobenzyl)pyrimidin-4-yl)oxy)piperidin-1-yl)methyl)-3-(oxetan-2-ylmethyl)-3H-imidazo[4,5-c]pyridine-6-carbimidate (120 mg, 0.20 mmol), DIEA (66 mg, 0.51 mmol) and formohydrazide (37 mg, 0.62 mmol) in n-BuOH (5 mL) was stirred at 120° C. for 3 hours. The reaction mixture was purified directly by prep-HPLC to give 2-[(4-chloro-2-fluorophenyl)methyl]-4-({1-[(3-{[(2S)-oxetan-2-yl]methyl}-6-(4H-1,2,4-triazol-3-yl)-3H-imidazo[4,5-c]pyridin-2-yl)methyl]piperidin-4-yl}oxy)pyrimidine Compound 166a (9 mg, yield: 7%) as a white solid.

MS Calcd.: 589.2; MS Found: 590.2 [M+H]⁺.

¹H NMR (400 MHz, DMSO-d₆): δ 9.10 (s, 1H), 8.40 (d, J=6.0 Hz, 1H), 8.24 (s, 1H), 8.03 (brs, 1H), 7.35-7.44 (m, 2H), 7.22-7.27 (m, 1H), 6.73 (d, J=5.6 Hz, 1H), 5.08-5.17 (m, 1H), 4.85-4.93 (m, 2H), 4.68-4.76 (m, 1H), 4.47-4.55 (m, 1H), 4.36-4.44 (m, 1H), 4.15 (s, 2H), 3.99 (d, J=14.0 Hz, 1H), 3.85 (d, J=14.0 Hz, 1H), 2.65-2.83 (m, 3H), 2.38-2.52 (m, 1H), 2.26-2.36 (m, 2H), 1.82-1.94 (m, 2H), 1.53-1.67 (m, 2H). ¹⁹F-NMR (377 MHz): −114.06.

### Example 51: 3-(2-{[4-({2-[(4-chloro-2-fluorophenyl)methyl]pyrimidin-4-yl}oxy)piperidin-1-yl]methyl}-3-{[(2S)-oxetan-2-yl]methyl}-3H-imidazo[4,5-c]pyridin-6-yl)-4,5-dihydro-1,2,4-oxadiazol-5-one (Compound 167a)

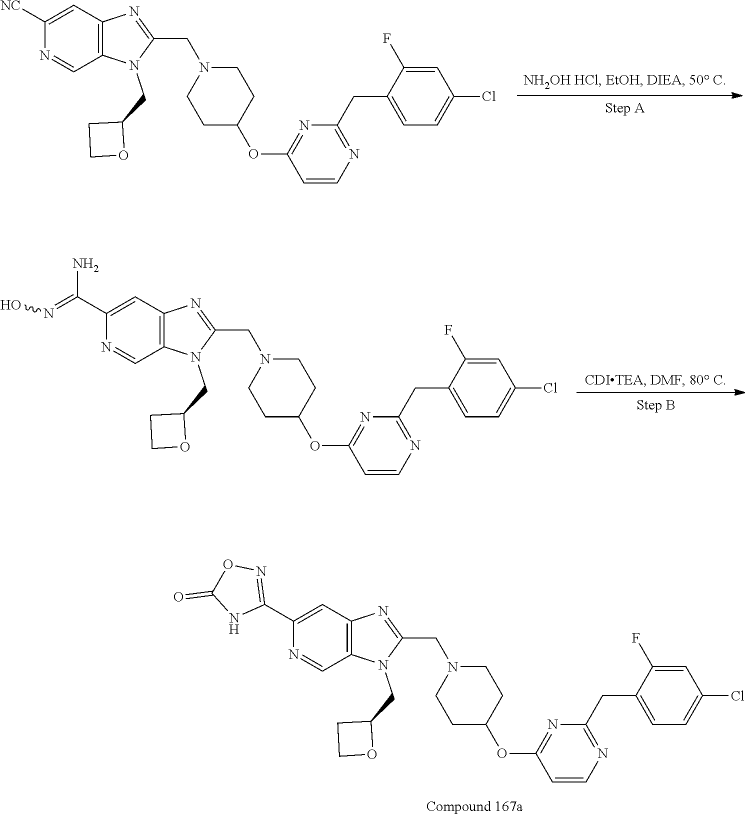 [SMILES: N#Cc1cc2nc(CN3CCC(Oc4ccnc(Cc5ccc(Cl)cc5F)n4)CC3)n(C[C@@H]3CCO3)c2cn1 ; NC(=NO)c1cc2nc(CN3CCC(Oc4ccnc(Cc5ccc(Cl)cc5F)n4)CC3)n(C[C@@H]3CCO3)c2cn1 ; O=c1[nH]c(-c2cc3nc(CN4CCC(Oc5ccnc(Cc6ccc(Cl)cc6F)n5)CC4)n(C[C@@H]4CCO4)c3cn2)no1]

### Step A: (S)-2-((4-((2-(4-chloro-2-fluorobenzyl)pyrimidin-4-yl)oxy)piperidin-1-yl)methyl)-N′-hydroxy-3-(oxetan-2-ylmethyl)-3H-imidazo[4,5-c]pyridine-6-carboximidamide

A mixture of (S)-2-((4-((2-(4-chloro-2-fluorobenzyl)pyrimidin-4-yl)oxy)piperidin-1-yl)methyl)-3-(oxetan-2-ylmethyl)-3H-imidazo[4,5-c]pyridine-6-carbonitrile (50 mg, 0.09 mmol), hydroxylamine hydrochloride (19 mg, 0.27 mmol) and DIEA (37 mg, 0.27 mmol) in EtOH (4 mL) was stirred at 50° C. for 4 hours. The mixture was diluted with EtOAc (10 mL), washed with water (10 mL×2). The organic layer was dried over sodium sulfate, filtered, and concentrated under reduced pressure to get (S)-2-((4-((2-(4-chloro-2-fluorobenzyl)pyrimidin-4-yl)oxy)piperidin-1-yl)methyl)-N′-hydroxy-3-(oxetan-2-ylmethyl)-3H-imidazo[4,5-c]pyridine-6-carboximidamide (56 mg, crude) as a white solid. MS Calcd.: 580.2; MS Found: 580.9 [M+H]⁺.

### Step B: 3-(2-{[4-({2-[(4-chloro-2-fluorophenyl)methyl]pyrimidin-4-yl}oxy)piperidin-1-yl]methyl}-3-{[(2S)-oxetan-2-yl]methyl}-3H-imidazo[4,5-c]pyridin-6-yl)-4,5-dihydro-1,2,4-oxadiazol-5-one (Compound 167a)

A mixture of (S)-2-((4-((2-(4-chloro-2-fluorobenzyl)pyrimidin-4-yl)oxy)piperidin-1-yl)methyl)-N′-hydroxy-3-(oxetan-2-ylmethyl)-3H-imidazo[4,5-c]pyridine-6-carboximidamide (56 mg), CDI (46 mg, 0.29 mmol) and TEA (30 mg, 0.29 mmol) in DMF (3 ml) was stirred at 80° C. for 3 hours. The reaction mixture was purified directly by prep-HPLC to give 3-(2-{[4-({2-[(4-chloro-2-fluorophenyl)methyl]pyrimidin-4-yl}oxy)piperidin-1-yl]methyl}-3-{[(2S)-oxetan-2-yl]methyl}-3H-imidazo[4,5-c]pyridin-6-yl)-4,5-dihydro-1,2,4-oxadiazol-5-one Compound 167a (7 mg, yield: 13%) as a white solid.

MS Calcd.: 606.2; MS Found: 607.2 [M+H]⁺.

¹H NMR (400 MHz, DMSO-d₆): δ 9.15 (s, 1H), 8.40 (d, J=6.0 Hz, 1H), 8.16 (s, 1H), 7.35-7.44 (m, 2H), 7.21-7.26 (m, 1H), 6.72 (d, J=6.0 Hz, 1H), 5.10-5.18 (m, 1H), 4.83-4.94 (m, 2H), 4.67-4.75 (m, 1H), 4.47-4.55 (m, 1H), 4.37-4.45 (m, 1H), 4.15 (s, 2H), 3.94-4.02 (m, 1H), 3.81-3.87 (m, 1H), 2.63-2.82 (m, 3H), 2.38-2.52 (m, 1H), 2.27-2.38 (m, 2H), 1.82-1.94 (m, 2H), 1.52-1.67 (m, 2H). ¹⁹F-NMR (377 MHz): −114.07.

### Example 52: 2-[(4-chloro-2-fluorophenyl)methyl]-5-fluoro-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine (Compound 168a)

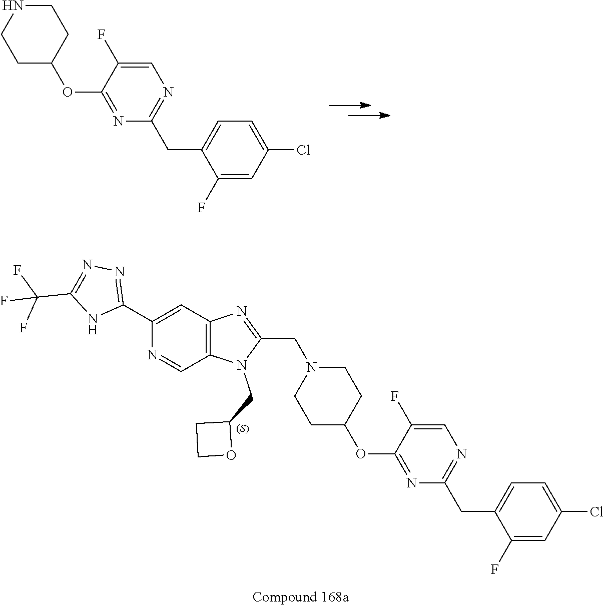

2-[(4-chloro-2-fluorophenyl)methyl]-5-fluoro-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine Compound 168a (32 mg) was obtained with the similar procedure of Compound 149a as a white solid.

MS Calcd.: 675.2; MS Found: 676.1 [M+H]⁺.

¹H NMR (400 MHz, DMSO-d₆): δ 9.17 (s, 1H), 8.51 (d, J=3.2 Hz, 1H), 8.29 (s, 1H), 7.36-7.43 (m, 2H), 7.25 (dd, J=8.4 Hz, 1.6 Hz, 1H), 5.11-5.20 (m, 1H), 4.95-5.04 (m, 1H), 4.87-4.95 (m, 1H), 4.71-4.77 (m, 1H), 4.48-4.56 (m, 1H), 4.38-4.45 (m, 1H), 4.15 (s, 2H), 3.94-4.07 (m, 1H), 3.79-3.94 (m, 1H), 2.65-2.87 (m, 3H), 2.27-2.52 (m, 3H), 1.88-2.00 (m, 2H), 1.60-1.75 (s, 2H). ¹⁹F-NMR (377 MHz): −63.73, −114.14, 157.35.

### Example 53: 2-[(4-chloro-2-fluorophenyl)methyl]-5-fluoro-4-({1-[(3-{[(2S)-oxetan-2-yl]methyl}-6-(4H-1,2,4-triazol-3-yl)-3H-imidazo[4,5-c]pyridin-2-yl)methyl]piperidin-4-yl}oxy)pyrimidine (Compound 169a)

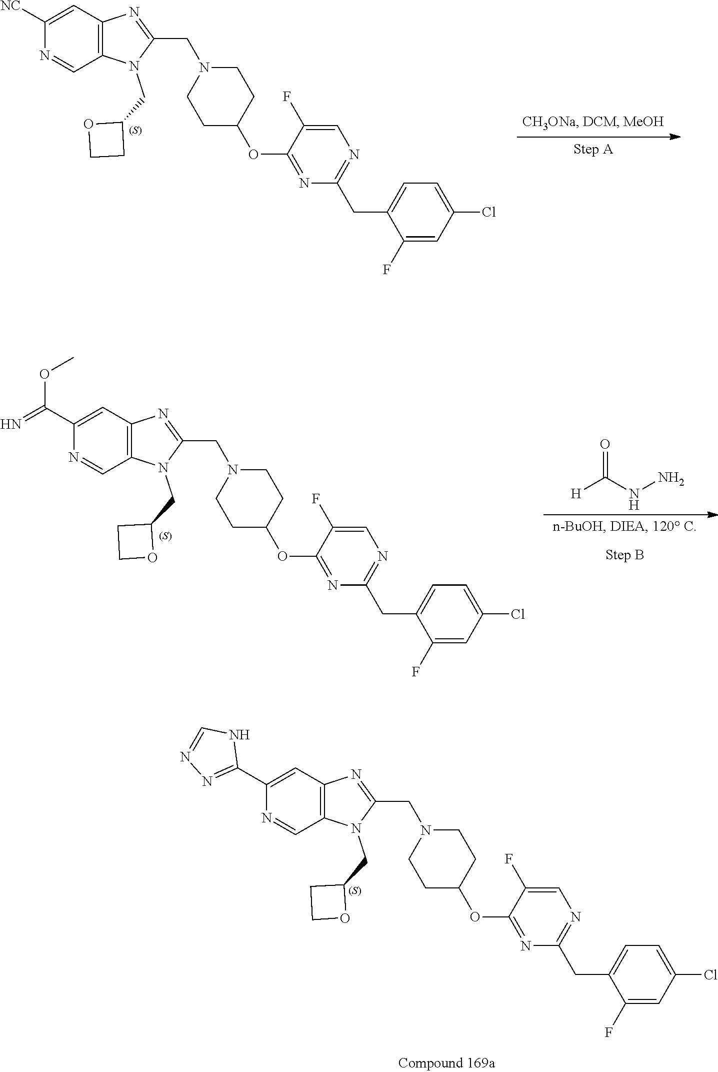

### Step A: methyl (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbimidate

To a solution of (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbonitrile (170 mg, 0.30 mmol) in DCM (2 mL) and MeOH (2 mL) was added sodium methanolate (162 mg, 3.0 mmol). The solution was stirred at room temperature for 4 hours. The reaction was quenched with water (10 mL), extracted with ethyl acetate (10 mL×2). The organic layers were combined, dried over Na₂SO₄, filtered and concentrated under vacuum. The residue was purified by Prep-TLC (DCM/MeOH=20/1) to give methyl (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbimidate (90 mg, yield: 50%) as a white solid. MS Calcd.: 597.2; MS Found: 598.7 [M+H]⁺.

### Step B: 2-[(4-chloro-2-fluorophenyl)methyl]-5-fluoro-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine (Compound 169a)

To a solution of methyl (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbimidate (45 mg, 0.075 mmol) in n-BuOH (1.5 mL) was added formohydrazide (9.0 mg, 0.15 mmol) and DIEA (29 mg, 0.23 mmol). The solution was stirred at 120° C. for 16 hours. The solvent was removed in vacuo. The residue was purified by Prep-HPLC to give 2-[(4-chloro-2-fluorophenyl)methyl]-5-fluoro-4-({1-[(3-{[(2S)-oxetan-2-yl]methyl}-6-(4H-1,2,4-triazol-3-yl)-3H-imidazo[4,5-c]pyridin-2-yl)methyl]piperidin-4-yl}oxy)pyrimidine Compound 169a (10 mg, yield: 22%) as a white solid.

MS Calcd.: 607.2; MS Found: 608.1 [M+H]⁺.

¹H NMR (400 MHz, DMSO-d₆): δ 9.26 (s, 1H), 8.57 (d, J=2.8 Hz, 1H), 8.37 (s, 1H), 8.25 (brs, 1H), 7.37-7.45 (m, 2H), 7.25 (dd, J=8.0 Hz, 2.0 Hz, 1H), 5.20-5.32 (m, 1H), 5.07-5.15 (m, 1H), 4.81-5.00 (m, 3H), 4.72-4.79 (m, 1H), 4.48-4.56 (m, 1H), 4.36-4.43 (m, 1H), 4.16 (s, 2H), 3.35-3.59 (m, 4H), 2.70-2.82 (m, 1H), 2.30-2.41 (m, 1H), 2.18-2.30 (m, 2H), 2.00-2.15 (m, 2H). ¹⁹F-NMR (377 MHz): −114,087, −156.78.

### Example 54: 2-[(4-chloro-2-fluorophenyl)methyl]-5-fluoro-4-[(1-{[6-(5-methyl-4H-1,2,4-triazol-3-yl)-3-{[(2S)-oxetan-2-yl]methyl}-3H-imidazo[4,5-c]pyridin-2-yl]methyl}piperidin-4-yl)oxy]pyrimidine (Compound 170a)

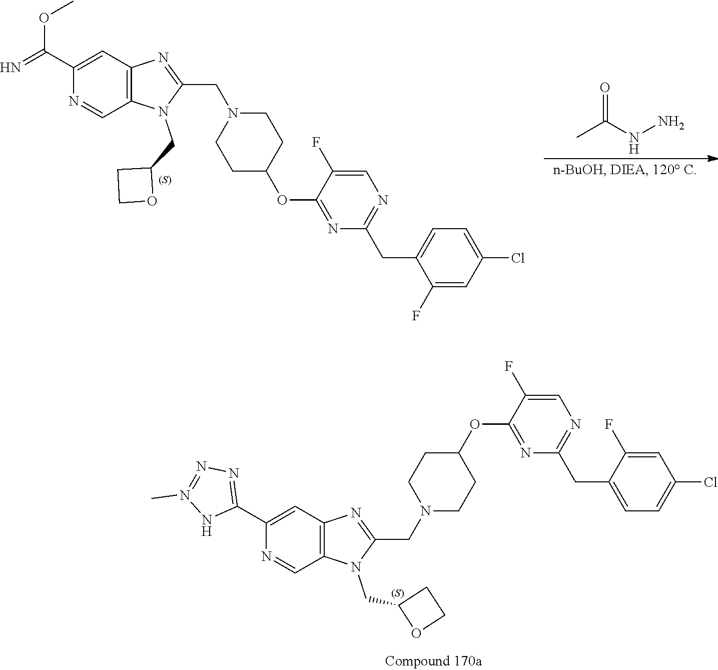

To a solution of methyl (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbimidate (45 mg, 0.075 mmol) in n-BuOH (1.5 mL) was added acetohydrazide (11 mg, 0.15 mmol) and DIEA (29 mg, 0.23 mmol). The solution was stirred at 120° C. for 16 hours. The solvent was removed in vacuo. The residue was purified by Prep-HPLC to give 2-[(4-chloro-2-fluorophenyl)methyl]-5-fluoro-4-[(1-{[6-(5-methyl-4H-1,2,4-triazol-3-yl)-3-f{[(2S)-oxetan-2-yl]methyl}-3H-imidazo[4,5-c]pyridin-2-yl]methyl}piperidin-4-yl)oxy]pyrimidine Compound 170a (9.5 mg, yield: 20%) as a white solid.

MS Calcd.: 621.2; MS Found: 622.0 [M+H]⁺.

¹H NMR (400 MHz, DMSO-d₆): δ14.08 (s, 1H), 9.07 (s, 1H), 8.51 (d, J=3.2 Hz, 1H), 8.18 (s, 1H), 7.37-7.43 (m, 2H), 7.25 (dd, J=8.4 Hz, 2.0 Hz, 1H), 5.10-5.18 (m, 1H), 4.94-5.03 (m, 1H), 4.82-4.91 (m, 1H), 4.68-4.75 (m, 1H), 4.48-4.56 (m, 1H), 4.38-4.47 (m, 1H), 4.15 (s, 2H), 3.98 (d, J=13.6 Hz, 1H), 3.84 (d, J=14.0 Hz, 1H), 2.67-2.84 (m, 3H), 2.25-2.52 (m, 6H), 1.86-1.97 (m, 2H), 1.60-1.73 (m, 2H). ¹⁹F-NMR (377 MHz): −114.14, −157.36.

### Example 55: 3-(2-{[4-({2-[(4-chloro-2-fluorophenyl)methyl]-5-fluoropyriimidin-4-yl}oxy)piperidin-1-yl]methyl}-3-{[(2S)-oxetan-2-yl]methyl}-3H-imidazo[4,5-c]pyridin-6-yl)-4,5-dihydro-1,2,4-oxadiazol-5-one (Compound 171a)

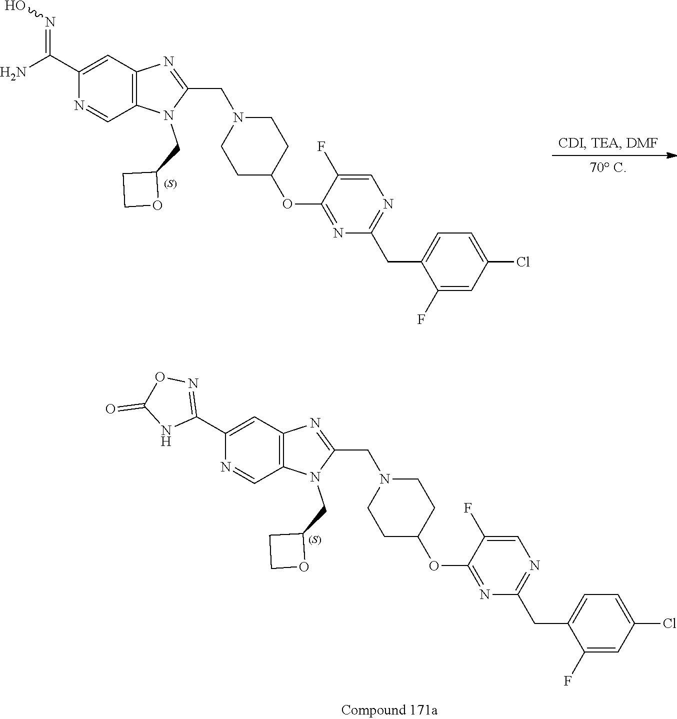

To a solution of (S)-2-((4-((2-(4-chloro-2-fluorobenzyl)-5-fluoropyrimidin-4-yl)oxy)piperidin-1-yl)methyl)-N′-hydroxy-3-(oxetan-2-ylmethyl)-3H-imidazo[4,5-c]pyridine-6-carboximidamide (35 mg) in DMF (1.5 mL) was added CDI (19 mg, 0.12 mmol) and TEA (17 mg, 0.17 mmol). The solution was stirred at 70° C. for an hour. The solvent was removed in vacuo. The residue was purified by Prep-HPLC to afford 3-(2-{[4-({2-[(4-chloro-2-fluorophenyl)methyl]-5-fluoropyrimidin-4-yl}oxy)piperidin-1-yl]methyl}-3-{[(2S)-oxetan-2-yl]methyl}-3H-imidazo[4,5-c]pyridin-6-yl)-4,5-dihydro-1,2,4-oxadiazol-5-one Compound 171a (7 mg) as a white solid.

MS Calcd.: 624.2; MS Found: 625.0 [M+H]⁺.

¹H NMR (400 MHz, DMSO-d₆): δ 9.14 (s, 1H), 8.50 (d, J=3.2 Hz, 1H), 8.16 (s, 1H), 7.36-7.42 (m, 2H), 7.25 (dd, J=8.0 Hz, 1.6 Hz, 1H), 5.09-5.18 (m, 1H), 4.94-5.04 (m, 1H), 4.85-4.93 (m, 1H), 4.69-4.76 (m, 1H), 4.46-4.55 (m, 1H), 4.38-4.44 (m, 1H), 4.15 (s, 2H), 3.99 (d, J=14.0 Hz, 1H), 3.86 (d, J=14.0 Hz, 1H), 2.65-2.84 (m, 3H), 2.29-2.52 (m, 3H), 1.86-1.98 (m, 2H), 1.59-1.73 (m, 2H). ¹⁹F-NMR (377 MHz): −114.13, −157.36.

### Example 56: 2-[(4-chloro-2-fluorophenyl)methyl]-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine (Compound 138a)

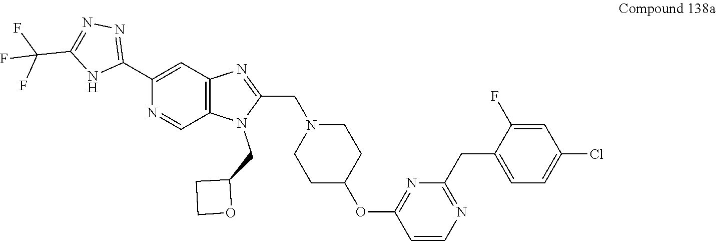 [SMILES: Fc1cc(Cl)ccc1Cc1nccc(OC2CCN(Cc3nc4cc(-c5nnc(C(F)(F)F)[nH]5)ncc4n3C[C@@H]3CCO3)CC2)n1]

2-[(4-chloro-2-fluorophenyl)methyl]-4-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyrimidine Compound 138a (37 mg) was obtained with the similar procedure of Compound 149a.

MS Calcd.: 657.2; MS Found: 658.3 [M+H]⁺.

¹H NMR (400 MHz, DMSO-d₆): δ 9.16 (s, 1H), 8.41 (d, J=5.6 Hz, 1H), 8.28 (d, J=0.8 Hz, 1H), 7.36-7.43 (m, 2H), 7.25 (dd, J=8.0, 1.6 Hz, 1H), 6.73 (d, J=4.8 Hz, 1H), 5.10-5.20 (m, 1H), 4.86-4.95 (m, 2H), 4.70-4.80 (m, 1H), 4.47-4.57 (m, 1H), 4.37-4.46 (m, 1H), 4.15 (s, 2H), 3.99 (d, J=13.6 Hz, 1H), 3.85 (d, J=14.0 Hz, 1H), 2.64-2.82 (m, 3H), 2.40-2.53 (m, 1H), 2.27-2.36 (m, 2H), 1.83-1.95 (m, 2H), 1.54-1.67 (m, 2H). ¹⁹F-NMR (377 MHz): −63.70, −114.06.

### Example 57: 3-(1-{[(2S)-oxetan-2-yl]methyl}-2-{[(1r,4r)-4-({2-[(4-chlorophenyl)methyl]-5-fluoropyrimidin-4-yl}oxy)cyclohexyl]methyl}-1H-1,3-benzodiazol-5-yl)-4,5-dihydro-1,2,4-oxadiazol-5-one (Compound 172a)

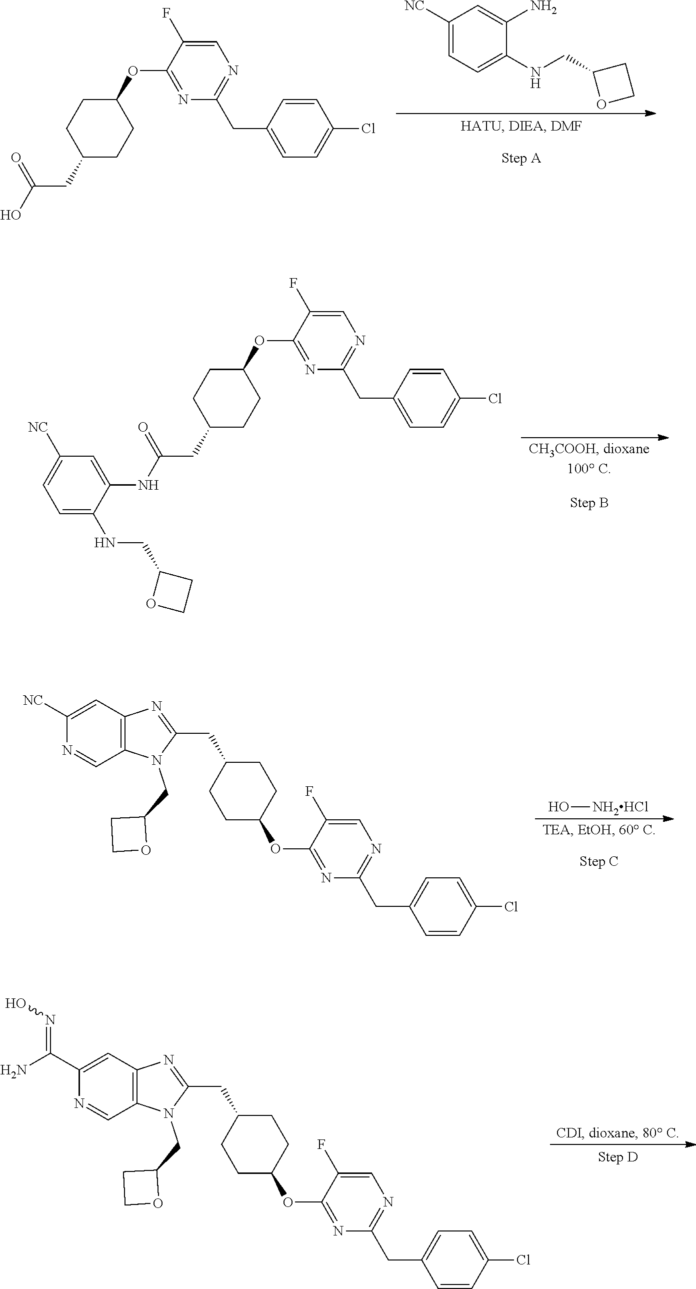

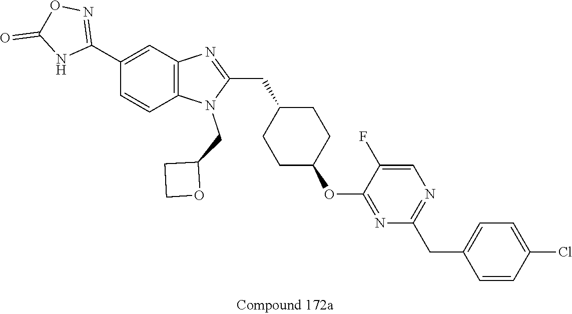 [SMILES: O=c1[nH]c(-c2ccc3c(c2)nc(C[C@H]2CC[C@H](Oc4nc(Cc5ccc(Cl)cc5)ncc4F)CC2)n3C[C@@H]2CCO2)no1]

### Step A: 2-((1r,4s)-4-((2-(4-chlorobenzyl)-5-fluoropyrimidin-4-yl)oxy)cyclohexyl)-N-(5-cyano-2-((((S)-oxetan-2-yl)methyl)amino)phenyl)acetamide

To a solution of 2-((1r,4r)-4-((2-(4-chlorobenzyl)-5-fluoropyrimidin-4-yl)oxy)cyclohexyl)acetic acid (205 mg, 0.54 mmol), (S)-3-amino-4-((oxetan-2-ylmethyl)amino)benzonitrile (100 mg, 0.49 mmol) and DIEA (127 mg, 0.98 mmol) in DMF (3 mL) was added HATU (224.6 mg, 0.59 mmol). The reaction was stirred at room temperature for 1 hour. After the reaction was completed, the reaction was quenched with water (30 mL) and extracted with ethyl acetate (50 mL×3). The organic layer was combined and washed with brine (30 mL×2), dried over sodium sulfate, filtered, and concentrated in vacuum. The residue was purified by column chromatography (PE:EA=1:1) to give 2-((1r,4s)-4-((2-(4-chlorobenzyl)-5-fluoropyrimidin-4-yl)oxy)cyclohexyl)-N-(5-cyano-2-((((S)-oxetan-2-yl)methyl)amino)phenyl)acetamide (240 mg, 87% yield) as yellow oil. MS Calcd.: 563.2; MS Found: 586.2 [M+Na]⁺.

### Step B: 2-(((1r,4s)-4-((2-(4-chlorobenzyl)-5-fluoropyrimidin-4-yl)oxy)cyclohexyl)methyl)-1-(((S)-oxetan-2-yl)methyl)-1H-benzo[d]imidazole-5-carbonitrile

To a solution of 2-((1r,4s)-4-((2-(4-chlorobenzyl)-5-fluoropyrimidin-4-yl)oxy) cyclohexyl)-N-(5-cyano-2-((((S)-oxetan-2-yl)methyl)amino)phenyl)acetamide (240 mg, 0.43 mmol) in dioxane (9 mL) was added CH₃COOH (3 mL). The reaction was stirred at 100° C. for 40 hours. After the reaction was completed, the reaction was concentrated in vacuum. The residue was purified by column chromatography (PE:EA=1:1) to give 2-(((1r,4s)-4-((2-(4-chlorobenzyl)-5-fluoropyrimidin-4-yl)oxy)cyclohexyl)methyl)-1-(((S)-oxetan-2-yl)methyl)-1H-benzo[d]imidazole-5-carbonitrile (180 mg, 78% yield) as a yellow solid. MS Calcd.: 545.2; MS Found: 546.2 [M+H]⁺.

### Step C: 2-(((1r,4s)-4-((2-(4-chlorobenzyl)-5-fluoropyrimidin-4-yl)oxy)cyclohexyl)methyl)-N′-hydroxy-1-(((S)-oxetan-2-yl)methyl)-1H-benzo[d]imidazole-5-carboximidamide

To a solution of 2-(((1r,4s)-4-((2-(4-chlorobenzyl)-5-fluoropyrimidin-4-yl)oxy)cyclohexyl)methyl)-1-(((S)-oxetan-2-yl)methyl)-1H-benzo[d]imidazole-5-carbonitrile (180 mg, 0.33 mmol) in EtOH (3 mL) was added TEA (100 mg, 0.99 mmol) and hydroxylamine hydrochloride (46 mg, 0.66 mmol) at room temperature. The reaction was stirred at 60° C. for 1 hour. After the reaction was completed, the reaction was filtered and the solid phase was collected to give 2-(((1r,4s)-4-((2-(4-chlorobenzyl)-5-fluoropyrimidin-4-yl)oxy)cyclohexyl)methyl)-N′-hydroxy-1-(((S)-oxetan-2-yl)methyl)-1H-benzo[d]imidazole-5-carboximidamide (170 mg, 89% yield) as a white solid. MS Calcd.: 578.2; MS Found: 579.2 [M+H]⁺.

### Step D: 3-(1-{[(2S)-oxetan-2-yl]methyl}-2-{[(1r,4r)-4-({2-[(4-chlorophenyl)methyl]-5-fluoropyrimidin-4-yl}oxy)cyclohexyl]methyl}-1H-1,3-benzodiazol-5-yl)-4,5-dihydro-1,2,4-oxadiazol-5-one (Compound 172a)

To a solution of 2-(((1r,4s)-4-((2-(4-chlorobenzyl)-5-fluoropyrimidin-4-yl)oxy)cyclohexyl)methyl)-N′-hydroxy-1-(((S)-oxetan-2-yl)methyl)-1H-benzo[d]imidazole-5-carboximidamide (70 mg) in dioxane (2 mL) was added TEA (24 mg, 0.24 mmol) and CDI (29 mg, 0.18 mmol) at room temperature. The reaction was stirred at 80° C. for 3 hours. After the reaction was completed, the reaction was filtered and purified by Prep-HPLC to give 3-(1-{[(2S)-oxetan-2-yl]methyl}-2-{[(1r,4r)-4-({2-[(4-chlorophenyl)methyl]-5-fluoropyrimidin-4-yl}oxy)cyclohexyl]methyl}-1H-1,3-benzodiazol-5-yl)-4,5-dihydro-1,2,4-oxadiazol-5-one Compound 172a (22 mg, 76% yield) as yellow solid.

MS Calcd.: 604.2; MS Found: 605.1 [M+H]⁺.

¹H NMR (400 MHz, DMSO-d₆): δ 8.48 (d, J=3.6 Hz, 1H), 8.01 (d, J=1.2 Hz, 1H), 7.75 (d, J=8.4 Hz, 1H), 7.66 (dd, J=8.4 Hz, J=1.2 Hz, 1H), 7.30-7.37 (m, 4H), 4.96-5.07 (m, 2H), 4.58-4.66 (m, 1H), 4.44-4.53 (m, 2H), 4.30-4.36 (m, 1H), 4.09 (s, 2H), 2.82-2.96 (m, 2H), 2.65-2.75 (m, 1H), 2.31-2.41 (m, 1H), 1.97-2.08 (m, 3H), 1.83-1.91 (m, 2H), 1.39-1.51 (m, 2H), 1.15-1.31 (m, 2H). ¹⁹F-NMR (377 MHz): −157.55.

### Example 58: 1-{[(2S)-oxetan-2-yl]methyl}-2-{[(1r,4r)-4-({2-[(4-chlorophenyl)methyl]-5-fluoropyrimidin-4-yl}oxy)cyclohexyl]methyl}-5-[5-(trifluoromethyl)-4H-1,2,4-triazol-3-yl]-1H-1,3-benzodiazole (Compound 173a)

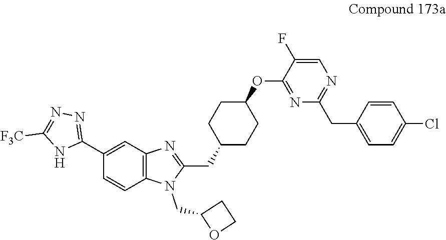 [SMILES: Fc1cnc(Cc2ccc(Cl)cc2)nc1O[C@H]1CC[C@H](Cc2nc3cc(-c4nnc(C(F)(F)F)[nH]4)ccc3n2C[C@@H]2CCO2)CC1]

1-{[(2S)-oxetan-2-yl]methyl}-2-{[(1r,4r)-4-({2-[(4-chlorophenyl)methyl]-5-fluoropyrimidin-4-yl}oxy)cyclohexyl]methyl}-5-[5-(trifluoromethyl)-4H-1,2,4-triazol-3-yl]-1H-1,3-benzodiazole Compound 173a (39 mg) was obtained with the similar procedure of Compound 149a as a white solid.

MS Calcd.: 655.2; MS Found: 656.1 [M+H]⁺.

¹H NMR (400 MHz, DMSO-d₆): δ 8.48 (d, J=3.2 Hz, 1H), 8.24 (d, J=0.8 Hz, 1H), 7.88 (dd, J=8.4 Hz, J=1.6 Hz, 1H), 7.76 (d, J=8.4 Hz, 1H), 7.30-7.38 (m, 4H), 4.96-5.08 (m, 2H), 4.58-4.66 (m, 1H), 4.45-4.53 (m, 2H), 4.31-4.38 (m, 1H), 4.09 (s, 2H), 2.83-2.97 (m, 2H), 2.65-2.76 (m, 1H), 2.30-2.46 (m, 1H), 2.00-2.10 (m, 3H), 1.84-1.92 (m, 2H), 1.40-1.52 (m, 2H), 1.19-1.32 (m, 2H). ¹⁹F-NMR (377 MHz): −63.66, −157.55.

### Example 59: 6-[(4-chloro-2-fluorophenyl)methoxy]-N-[(3R)-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]pyrrolidin-3-yl]pyridin-2-amine (Compound 174a)

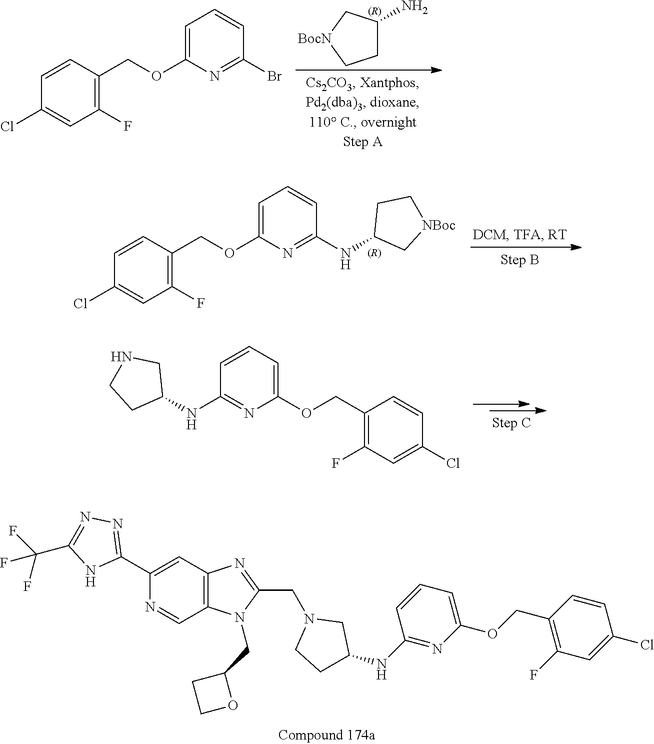

### Step A: tert-butyl (R)-3-((6-((4-chloro-2-fluorobenzyl)oxy)pyridin-2-yl)amino)pyrrolidine-1-carboxylate

First Batch: A mixture of 2-bromo-6-((4-chloro-2-fluorobenzyl)oxy)pyridine (50 mg, 0.16 mmol), tert-butyl (R)-3-aminopyrrolidine-1-carboxylate (30 mg, 0.16 mmol), Pd₂(dba)₃ (8 mg, 0.05 mmol), Cs₂CO₃ (104 mg, 0.32 mmol) and Xantphos (10 mg, 0.016 mmol) in Dioxane (3 ml) was stirred at 110° C. overnight under N₂ atmosphere.

Second Batch: A mixture of 2-bromo-6-((4-chloro-2-fluorobenzyl)oxy)pyridine (250 mg, 0.79 mmol), tert-butyl (R)-3-aminopyrrolidine-1-carboxylate (158 mg, 0.79 mmol), Pd(dba)₃ (36 mg, 0.04 mmol), CsCO₃ (514 mg, 2.4 mmol) and Xantphos (46 mg, 0.08 mmol) in Dioxane (5 ml) was stirred at 110° C. overnight under N₂ atmosphere. After the reaction was completed, the mixture was combined with the first batch, filtered. The filtrate was diluted with ethyl acetate (20 mL), washed with brine (20 mL×2). The organic layer was dried over sodium sulfate, filtered and concentrated in vacuum. The residue was purified by column chromatography on silica gel (PE/EA=8/1) to give tert-butyl (R)-3-((6-((4-chloro-2-fluorobenzyl)oxy)pyridin-2-yl)amino)pyrrolidine-1-carboxylate (400 mg, crude) as yellow oil. MS Calcd.: 421.2; MS Found: 422.3 [M+H]⁺.

### Step B: (R)-6-((4-chloro-2-fluorobenzyl)oxy)-N-(pyrrolidin-3-yl)pyridin-2-amine

First Batch: A mixture of tert-butyl (R)-3-((6-((4-chloro-2-fluorobenzyl)oxy)pyridin-2-yl)amino)pyrrolidine-1-carboxylate (50 mg, 0.12 mmol), and TFA (420 mg, 2.0 mmol) in DCM (2 mL) was stirred at room temperature for 1 hour.

Second Batch: A mixture of tert-butyl (R)-3-((6-((4-chloro-2-fluorobenzyl)oxy)pyridin-2-yl)amino)pyrrolidine-1-carboxylate (350 mg, 0.85 mmol), and TFA (840 mg, 4.0 mmol) in DCM (4 mL) was stirred at room temperature for 1 hour.

After the reaction was completed, the reaction mixture of the two batches was combined and concentrated in vacuum to give (R)-6-((4-chloro-2-fluorobenzyl)oxy)-N-(pyrrolidin-3-yl)pyridin-2-amine TFA salt (480 mg, crude) as yellow oil. MS Calcd.: 321.1. MS Found: 322.0 [M+H]⁺.

### Step C: 6-[(4-chloro-2-fluorophenyl)methoxy]-N-[(3R)-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]pyrrolidin-3-yl]pyridin-2-amine (Compound 174a)

6-[(4-chloro-2-fluorophenyl)methoxy]-N-[(3R)-1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]pyrrolidin-3-yl]pyridin-2-amine Compound 174a (27 mg) was obtained with the similar procedure of Compound 149a as a white solid.

MS Calcd.: 657.2; MS Found: 658.2 [M+H]⁺.

¹H NMR (400 MHz, DMSO-d6): δ 9.13 (d, J=0.8 Hz, 1H), 8.26 (d, J=0.8 Hz, 1H), 7.45 (t, J=8.0 Hz, 1H), 7.37 (dd, J=10.0 Hz, J=2.0 Hz, 1H), 7.29 (t, J=8.0 Hz, 1H), 7.24 (dd, J=8.0 Hz, J=1.6 Hz, 1H), 6.68 (d, J=6.4 Hz, 1H), 6.03 (d, J=8.0 Hz, 1H), 5.90 (d, J=7.6 Hz, 1H), 5.21-5.31 (m, 2H), 5.06-5.17 (m, 1H), 4.85-4.92 (m, 1H), 4.68-4.75 (m, 1H), 4.45-4.52 (m, 1H), 4.34-4.41 (m, 1H), 4.14-4.25 (m, 1H), 4.11 (d, J=13.6 Hz, 1H), 3.93 (d, J=13.6 Hz, 1H), 2.87-2.93 (m, 1H), 2.64-2.76 (m, 2H), 2.56-2.64 (m, 1H), 2.35-2.55 (m, 2H), 2.13-2.22 (m, 1H), 1.60-1.70 (m, 1H). ¹⁹F-NMR (377 MHz): −63.67, −115.64.

### Example 60: 2-[(4-chloro-2-fluorophenyl)methyl]-5-fluoro-4-({1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}oxy)pyrimidine (Compound 175a)

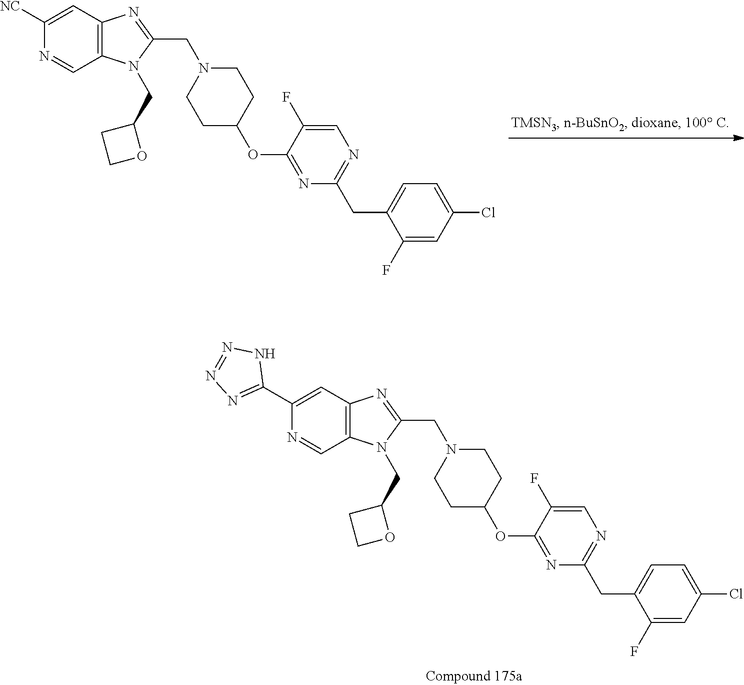 [SMILES: N#Cc1cc2nc(CN3CCC(Oc4nc(Cc5ccc(Cl)cc5F)ncc4F)CC3)n(C[C@@H]3CCO3)c2cn1 ; Fc1cc(Cl)ccc1Cc1ncc(F)c(OC2CCN(Cc3nc4cc(-c5nnn[nH]5)ncc4n3C[C@@H]3CCO3)CC2)n1]

A mixture of (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbonitrile (120 mg, 0.21 mmol), TMSN₃ (122 mg, 1.1 mmol) and n-BuSnO₂ (104.55 mg, 0.42 mmol) in dioxane (2.0 mL) was heated at 100° C. for 2 hours under an atmosphere of nitrogen. The solvent was removed in vacuo. The crude was purified by Prep-HPLC (0.1% FA/H₂O/CH₃CN) to give 2-[(4-chloro-2-fluorophenyl)methyl]-5-fluoro-4-({1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}oxy)pyrimidine Compound 175a (29 mg) as a white solid.

MS Calcd.: 608.2; MS Found: 609.2 [M+H]⁺.

¹H NMR (400 MHz, DMSO-d6): δ 9.20 (s, 1H), 8.50 (d, J=2.8 Hz, 1H), 8.38 (s, 1H), 7.36-7.43 (m, 2H), 7.24 (dd, J=8.0 Hz, 2.0 Hz, 1H), 5.10-5.20 (m, 1H), 4.94-5.04 (m, 1H), 4.86-4.94 (m, 1H), 4.71-4.78 (m, 1H), 4.47-4.56 (m, 1H), 4.36-4.45 (m, 1H), 4.15 (s, 2H), 4.01 (d, J=14.4 Hz, 1H), 3.88 (d, J=14.0 Hz, 1H), 2.68-2.85 (m, 3H), 2.30-2.51 (m, 3H), 1.86-1.97 (m, 2H), 1.60-1.74 (m, 2H). ¹⁹F-NMR (377 MHz): −114.14, −157.36.

### Example 61: 2-[(4-chloro-2-fluorophenyl)methyl]-6-({1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}oxy)pyridine (Compound 176a)

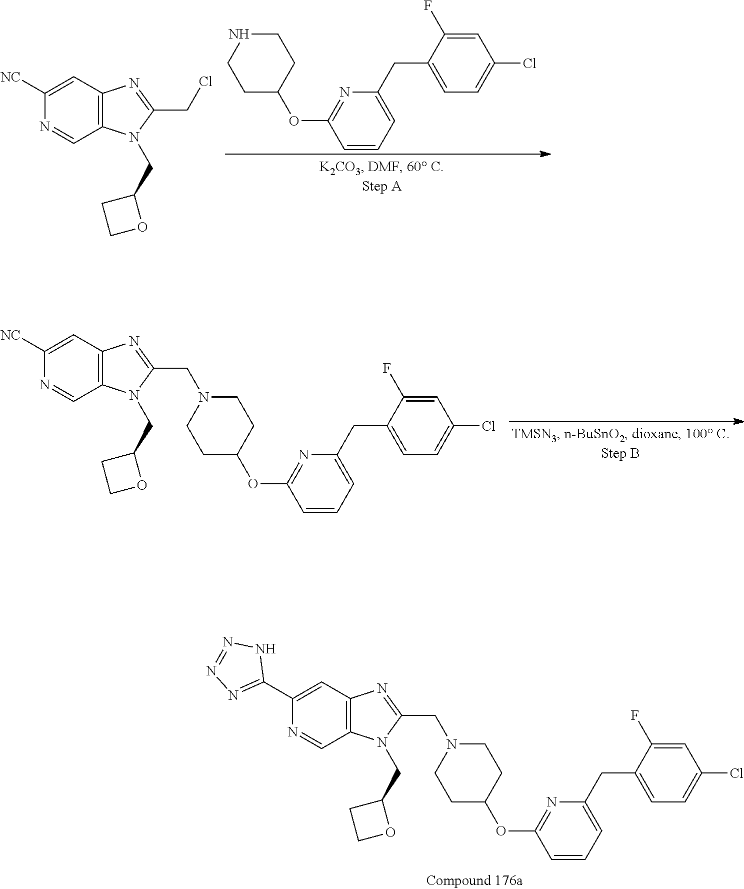

### Step A: (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbonitrile

A mixture of 2-(4-chloro-2-fluorobenzyl)-6-(piperidin-4-yloxy)pyridine (184 mg, 0.57 mmol), (S)-2-(chloromethyl)-3-(oxetan-2-ylmethyl)-3H-imidazo[4,5-c]pyridine-6-carbonitrile (60 mg, 0.23 mmol), K₂CO₃ (127 mg, 0.92 mmol) in DMF (4 mL) was stirred at 60° C. for 1 hour. After the reaction was completed, the mixture was diluted with EA (40 mL), washed with H₂O (30 mL*2). The combined organic layer was dried over anhydrous sodium sulfate, filtered, and evaporated to dryness. The residue was purified by column chromatography on silica gel (DCM/MeOH=80/1) to give (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbonitrile (95 mg, yield: 93%) as yellow oil. MS Calcd.: 546.2; MS Found: 547.2 [M+H]⁺.

### Step B: 2-[(4-chloro-2-fluorophenyl)methyl]-6-({1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}oxy)pyridine (Compound 176a)

A mixture of (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbonitrile (115 mg, 0.21 mmol), TMSN₃ (121 mg, 1.05 mmol) and Dibutyl oxostannane (104.55 mg, 0.42 mmol) in dioxane (2.0 mL) was heated at 100° C. for 2 hours under an atmosphere of nitrogen. The solvent was removed in vacuo. The crude was purified by Prep-HPLC (0.1% FA/H₂O/CH₃CN) to give 2-[(4-chloro-2-fluorophenyl)methyl]-6-({1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}oxy)pyridine Compound 176a (15 mg) as a white solid.

MS Calcd.: 589.2; MS Found: 590.2 [M+H]⁺.

¹H NMR (400 MHz, DMSO-d6): δ 9.20 (s, 1H), 8.40 (s, 1H), 7.60 (t, J=7.6 Hz, 1H), 7.32-7.43 (m, 2H), 7.23 (d, J=8.4 Hz, 1H), 6.82 (d, J=7.2 Hz, 1H), 6.58 (d, J=8.0 Hz, 1H), 5.12-5.23 (m, 1H), 4.73-4.95 (m, 3H), 4.47-4.56 (m, 1H), 4.38-4.45 (m, 1H), 3.97-4.07 (m, 3H), 3.87 (d, J=13.6 Hz, 1H), 2.70-2.85 (m, 3H), 2.27-2.53 (m, 3H), 1.82-1.92 (m, 2H), 1.50-1.63 (m, 2H). ¹⁹F-NMR (377 MHz): −114.57.

### Example 62: 2-[(4-chloro-2-fluorophenyl)methyl]-4-({1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}oxy)pyrimidine (Compound 177a)

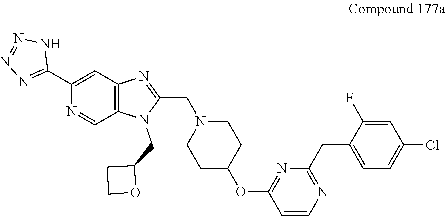 [SMILES: Fc1cc(Cl)ccc1Cc1nccc(OC2CCN(Cc3nc4cc(-c5nnn[nH]5)ncc4n3C[C@@H]3CCO3)CC2)n1]

2-(4-chloro-2-fluorobenzyl)-6-(piperidin-4-yloxy)pyridine. 2-[(4-chloro-2-fluorophenyl)methyl]-4-({1-[(3-{[(2S)-oxetan-2-yl]methyl}-6-(1H-1,2,3,4-tetrazol-5-yl)-3H-imidazo[4,5-c]pyridin-2-yl)methyl]piperidin-4-yl}oxy)pyrimidine Compound 177a (26 mg) was obtained as a white solid.

MS Calcd.: 590.2; MS Found: 591.3 [M+H]⁺.

¹H NMR (400 MHz, DMSO-d₆): δ 9.20 (s, 1H), 8.39-8.42 (m, 2H), 7.36-7.43 (m, 2H), 7.22-7.26 (m, 1H), 6.73 (d, J=5.6 Hz, 1H), 5.10-5.20 (m, 1H), 4.85-4.96 (m, 2H), 4.70-4.78 (m, 1H), 4.46-4.57 (m, 1H), 4.38-4.45 (m, 1H), 4.15 (s, 2H), 4.01 (d, J=13.2 Hz, 1H), 3.87 (d, J=13.6 Hz, 1H), 2.65-2.85 (m, 4H), 2.27-2.51 (m, 2H), 1.85-1.94 (m, 2H), 1.53-1.68 (m, 2H). ¹⁹F-NMR (377 MHz): −114.06.

### Example 63: 1-[(2-{[4-({2-[(4-chlorophenyl)methyl]-5-fluoropyrimidin-4-yl}oxy)piperidin-1-yl]methyl}-6-[5-(trifluoromethyl)-4H-1,2,4-triazol-3-yl]-3H-imidazo[4,5-c]pyridin-3-yl)methyl]cyclopropane-1-carbonitrile (Compound 178)

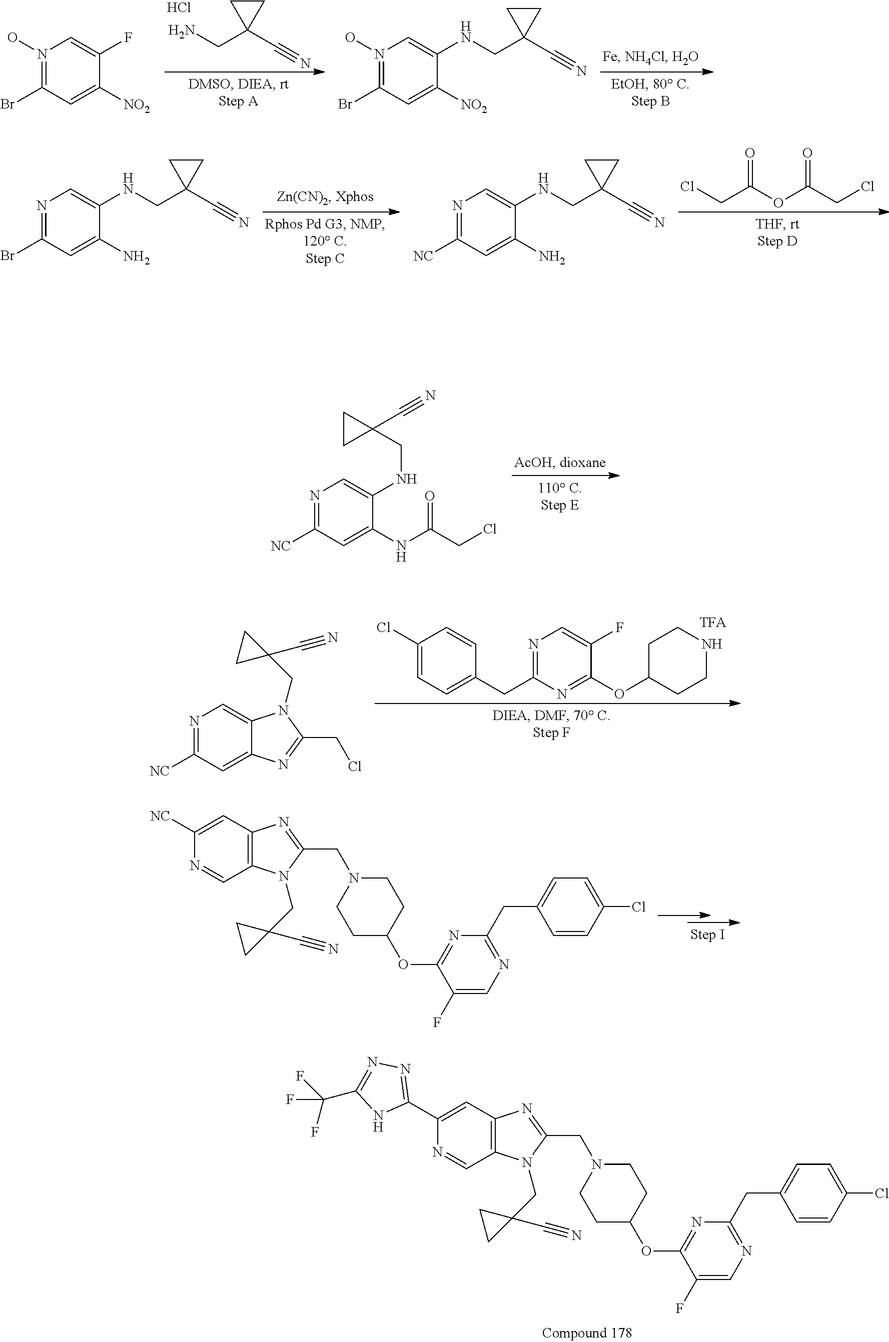

### Step A: 2-bromo-5-(((1-cyanocyclopropyl)methyl)amino)-4-nitropyridine 1-oxide

To a solution of 2-bromo-5-fluoro-4-nitropyridine 1-oxide (979 mg, 4.1 mmol) in DMSO (10 mL) was added 1-(aminomethyl)cyclopropane-1-carbonitrile (500 mg, HCl salt, 3.8 mmol) and DIEA (1.46 g, 11.3 mmol). The solution was stirred at room temperature for an hour. The reaction was quenched with water (20 mL), extracted with ethyl acetate (30 mL×2). The organic layers were combined, dried over Na₂SO₄, filtered and concentrated under vacuum. The residue was purified by silica gel column chromatography, eluting with PE:EA=2:1 to give 2-bromo-5-(((1-cyanocyclopropyl)methyl)amino)-4-nitropyridine 1-oxide (1.0 g, yield: 77%) as yellow oil. MS Calcd.: 312.0; MS Found: 313.0 [M+H]⁺.

### Step B: 1-(((4-amino-6-bromopyridin-3-yl)amino)methyl)cyclopropane-1-carbonitrile

To a mixture of 2-bromo-5-(((1-cyanocyclopropyl)methyl)amino)-4-nitropyridine 1-oxide (1.0 g, 3.2 mmol) in EtOH (8 mL) and H₂O (2 mL) was added NH₄Cl (857 mg, 16.0 mmol) and Fe (897 mg, 16.0 mmol). The mixture was stirred at 80° C. for 3 hours. The mixture was filtered. The filtrate was diluted with EA (50 mL) and washed with H₂O (15 mL). The organic layer was dried over Na₂SO₄, filtered, and concentrated under vacuum. The residue was purified by silica gel column chromatography, eluting with PE:EA=1:1 to give 1-(((4-amino-6-bromopyridin-3-yl)amino)methyl)cyclopropane-1-carbonitrile (350 mg, yield: 40%) as a purple solid. MS Calcd.: 266.0; MS Found: 266.9 [M+H]⁺.

### Step C: 4-amino-5-(((1-cyanocyclopropyl)methyl)amino)picolinonitrile

A mixture of 1-(((4-amino-6-bromopyridin-3-yl)amino)methyl)cyclopropane-1-carbonitrile (350 mg, 1.3 mmol), Zn(CN)₂ (462 mg, 3.9 mmol), Xphos (61 mg, 0.13 mmol) and Ruhos Pd G3 (110 mg, 0.13 mmol) in NMP (4 mL) was heated at 120° C. for 2 hours under an atmosphere of nitrogen. After the reaction was completed, the mixture was diluted with water (10 mL) and extracted with ethyl acetate (20 mL×2). The organic layers were combined, dried over Na₂SO₄, filtered, and concentrated under vacuum. The residue was purified by silica gel column chromatography, eluting with DCM:MeOH=10:1 to give 4-amino-5-(((1-cyanocyclopropyl)methyl)amino)picolinonitrile (200 mg, yield: 70%) as a white solid. MS Calcd.: 213.1; MS Found: 214.1 [M+H]⁺.

### Step D: 2-chloro-N-(2-cyano-5-(((1-cyanocyclopropyl)methyl)amino)pyridin-4-yl)acetamide

A mixture of 4-amino-5-(((1-cyanocyclopropyl)methyl)amino)picolinonitrile (200 mg, 0.94 mmol) and 2-chloroacetic anhydride (176.6 mg, 1.0 mmol) in THE (5 mL) was stirred at rt for 16 hours. The mixture was diluted with EA (15 mL), washed with saturated sodium bicarbonate solution (10 mL×2). The organic layer was dried over anhydrous sodium sulfate, filtered, and concentrated under vacuum to give 2-chloro-N-(2-cyano-5-(((1-cyanocyclopropyl)methyl)amino)pyridin-4-yl)acetamide (180 mg, yield: 66%) as a light-yellow solid. MS Calcd.: 289.1; MS Found: 290.1 [M+H]⁺.

### Step E: 2-(chloromethyl)-3-((1-cyanocyclopropyl)methyl)-3H-imidazo[4,5-c]pyridine-6-carbonitrile

To a solution of 2-chloro-N-(2-cyano-5-(((1-cyanocyclopropyl)methyl)amino)pyridin-4-yl)acetamide (170 mg, 0.39 mmol) in dioxane (6 mL) was added AcOH (0.8 mL). The solution was stirred at 110° C. for 16 hours. The reaction mixture was diluted with EA (20 mL), washed with saturated sodium bicarbonate solution (10 mL×2). The organic layer was dried over anhydrous sodium sulfate, filtered, and concentrated under vacuum to give 2-(chloromethyl)-3-((1-cyanocyclopropyl)methyl)-3H-imidazo[4,5-c]pyridine-6-carbonitrile (160 mg, yield: 95%, purity: 95%) as a brown solid. MS Calcd.: 271.1; MS Found: 272.0 [M+H]⁺.

### Step F: 2-((4-((2-(4-chlorobenzyl)-5-fluoropyrimidin-4-yl)oxy)piperidin-1-yl)methyl)-3-((1-cyanocyclopropyl)methyl)-3H-imidazo[4,5-c]pyridine-6-carbonitrile

To a solution of 2-(4-chlorobenzyl)-5-fluoro-4-(piperidin-4-yloxy)pyrimidine (153.5 mg, 0.35 mmol, TFA salt) in DMF (2 mL) was added DIEA (114 mg, 0.88 mmol). After 2 minutes, 2-(chloromethyl)-3-((1-cyanocyclopropyl)methyl)-3H-imidazo[4,5-c]pyridine-6-carbonitrile (80 mg, 0.294 mmol) was added. The resulting mixture was heated at 70° C. for an hour. The reaction was quenched with water (15 mL), extracted with ethyl acetate (20 mL×2). The organic layers were combined, dried over Na₂SO₄, filtered, and concentrated under vacuum. The residue was purified by Prep-TLC (DCM:MeOH=20:1) to give 2-((4-((2-(4-chlorobenzyl)-5-fluoropyrimidin-4-yl)oxy)piperidin-1-yl)methyl)-3-((1-cyanocyclopropyl)methyl)-3H-imidazo[4,5-c]pyridine-6-carbonitrile (100 mg, 61% yield) as a light-yellow solid. MS Calcd.: 556.2; MS Found: 557.2 [M+H]⁺.

### Step I: 1-[(2-{[4-({2-[(4-chlorophenyl)methyl]-5-fluoropyrimidin-4-yl}oxy)piperidin-1-yl]methyl}-6-[5-(trifluoromethyl)-4H-1,2,4-triazol-3-yl]-3H-imidazo[4,5-c]pyridin-3-yl)methyl]cyclopropane-1-carbonitrile (Compound 178)

1-[(2-{[4-({2-[(4-chlorophenyl)methyl]-5-fluoropyrimidin-4-yl}oxy)piperidin-1-yl]methyl}-6-[5-(trifluoromethyl)-4H-1,2,4-triazol-3-yl]-3H-imidazo[4,5-c]pyridin-3-yl)methyl]cyclopropane-1-carbonitrile Compound 178 (28 mg) was obtained with the similar procedure of Compound 149a as a white solid.

MS Calcd.: 666.2; MS Found: 667.0 [M+H]⁺.

¹H NMR (400 MHz, DMSO-d6): δ 9.29 (s, 1H), 8.51 (d, J=3.2 Hz, 1H), 8.34 (s, 1H), 7.29-7.38 (m, 4H), 5.06-5.18 (m, 1H), 4.84 (s, 2H), 4.09 (s, 2H), 4.00 (s, 2H), 2.72-2.87 (m, 2H), 2.33-2.50 (m, 2H), 1.91-2.05 (m, 2H), 1.65-1.80 (m, 2H), 1.42-1.54 (m, 4H). ¹⁹F-NMR (377 MHz): −63.72, −157.49.

### Example 64: 1-[(2-{[4-({6-[(4-chloro-2-fluorophenyl)methyl]pyridin-2-yl}oxy)piperidin-1-yl]methyl}-6-[5-(trifluoromethyl)-4H-1,2,4-triazol-3-yl]-3H-imidazo[4,5-c]pyridin-3-yl)methyl]cyclopropane-1-carbonitrile (Compound 179)

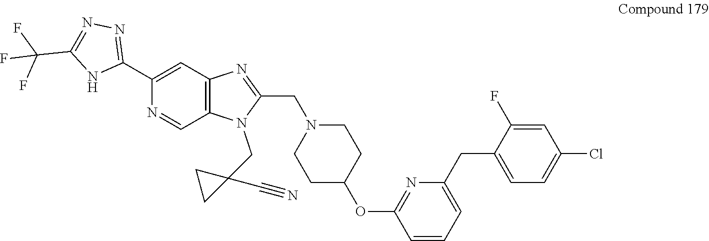 [SMILES: N#CC1(Cn2c(CN3CCC(Oc4cccc(Cc5ccc(Cl)cc5F)n4)CC3)nc3cc(-c4nnc(C(F)(F)F)[nH]4)ncc32)CC1]

2-(4-chlorobenzyl)-5-fluoro-4-(piperidin-4-yloxy)pyrimidine. 1-[(2-{[4-({6-[(4-chloro-2-fluorophenyl)methyl]pyridin-2-yl}oxy)piperidin-1-yl]methyl}-6-[5-(trifluoromethyl)-4H-1,2,4-triazol-3-yl]-3H-imidazo[4,5-c]pyridin-3-yl)methyl]cyclopropane-1-carbonitrile Compound 179 (31 mg) was obtained as a white solid.

MS Calcd.: 665.2; MS Found: 666.0 [M+H]⁺.

¹H NMR (400 MHz, DMSO-d6): δ 9.42 (s, 1H), 8.43 (s, 1H), 7.67 (t, J=7.6 Hz, 1H), 7.37-7.42 (m, 2H), 7.24 (dd, J=8.0 Hz, 2.0 Hz, 1H), 6.88 (d, J=7.2 Hz, 1H), 6.67 (d, J=8.4 Hz, 1H), 5.10 (brs, 1H), 4.98 (s, 2H), 4.84 (s, 2H), 4.02 (s, 2H), 3.23-3.65 (m, 4H), 2.10-2.24 (m, 2H), 1.84-2.08 (m, 2H), 1.48 (s, 4H). ¹⁹F-NMR (377 MHz): −63.71, −114.54.

### Example 65: 2-[(4-chloro-2-fluorophenyl)methyl]-5-fluoro-4-({1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}oxy)pyrimidine (Compound 180a)

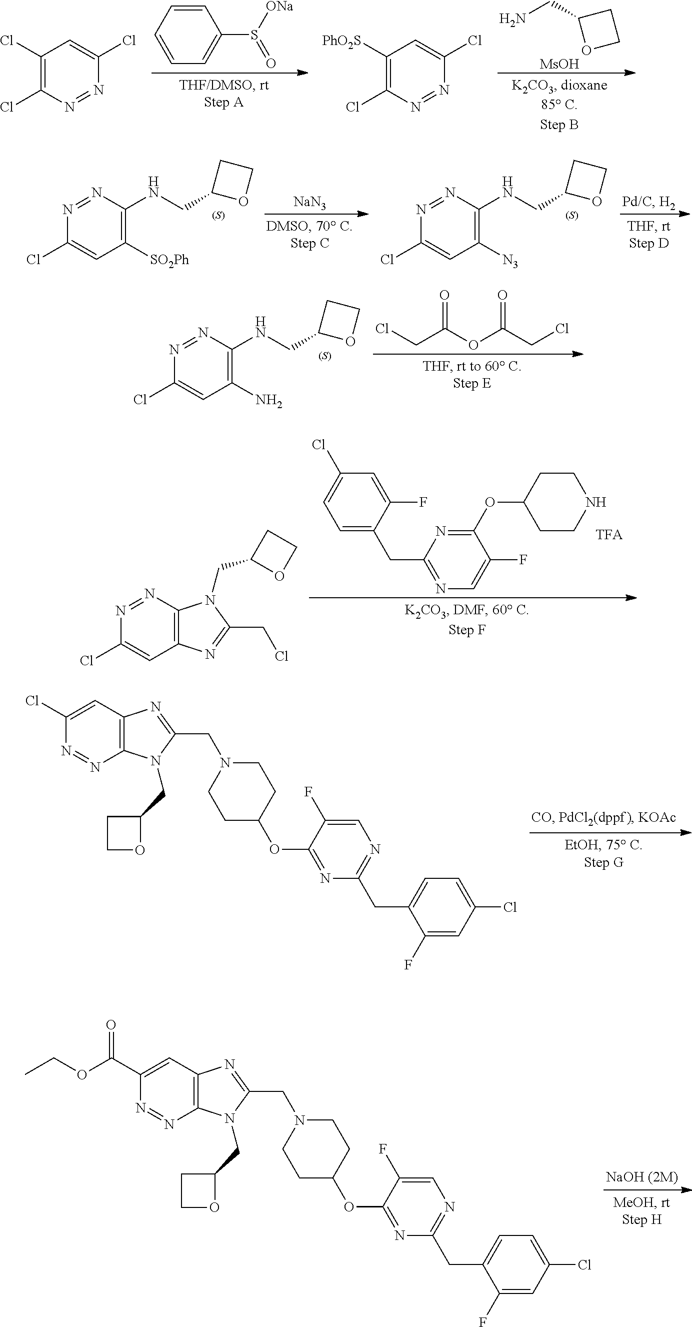

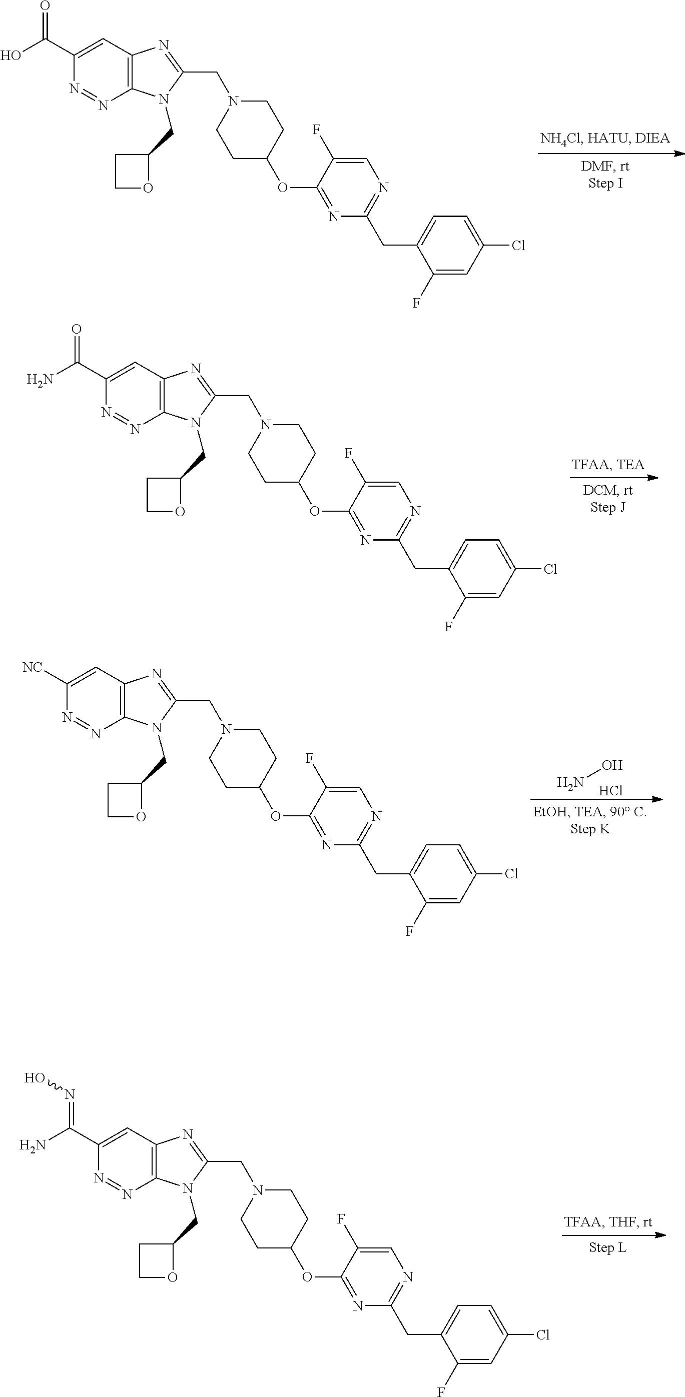

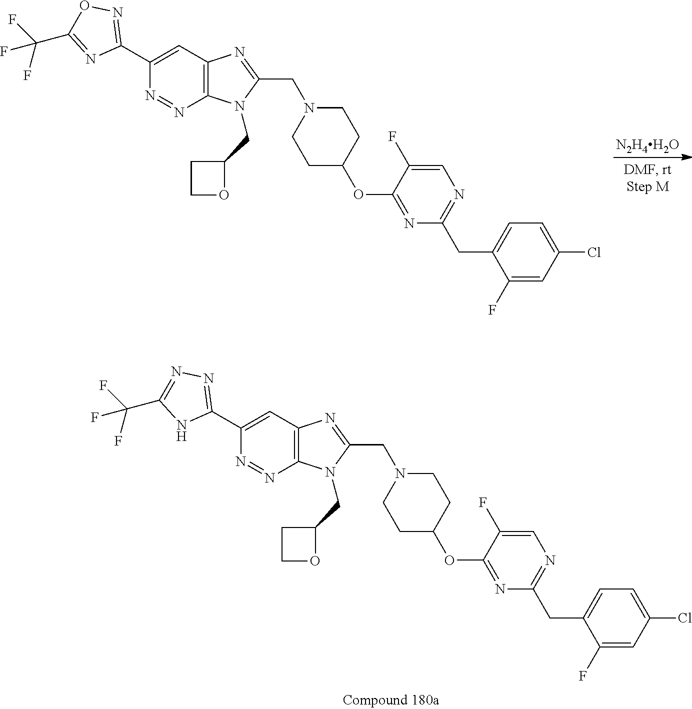 [SMILES: Fc1cc(Cl)ccc1Cc1ncc(F)c(OC2CCN(Cc3nc4cc(-c5noc(C(F)(F)F)n5)nnc4n3C[C@@H]3CCO3)CC2)n1 ; "N2H4·H2O" ; Fc1cc(Cl)ccc1Cc1ncc(F)c(OC2CCN(Cc3nc4cc(-c5nnc(C(F)(F)F)[nH]5)nnc4n3C[C@@H]3CCO3)CC2)n1]

### Step A: 3,6-dichloro-4-(phenylsulfonyl)pyridazine

To a solution of 3,4,6-trichloropyridazine (15.0 g, 81.8 mmol) in THF/DMSO (5:1) (120 mL) was added sodium benzenesulfinate (18.0 g, 90.0 mmol). The reaction mixture was stirred at room temperature for 1 hour. After the reaction was completed, the mixture was quenched with H₂O (100 mL) and extracted with EA (200 mL*2). The combined organic layers were dried over anhydrous sodium sulfate, filtered, and concentrated to dryness. The residue was purified by column chromatography on silica gel (PE/EA=10/1) to give 3,6-dichloro-4-(phenylsulfonyl)pyridazine (16.0 g, yield: 68%) as a white solid. MS Calcd.: 288.0; MS Found: 289.0 [M+H]⁺.

### Step B: (S)-6-chloro-N-(oxetan-2-ylmethyl)-4-(phenylsulfonyl)pyridazin-3-amine

A mixture of 3,6-dichloro-4-(phenylsulfonyl)pyridazine (5.0 g, 17.4 mmol), (S)-oxetan-2-ylmethanamine. MsOH salt (3.5 g, 19.1 mmol), K₂CO₃ (9.6 g, 69.4 mmol) in dioxane (100 mL) was stirred at 85° C. for 3 hours. After the reaction was completed, the reaction mixture was filtered, and the filtrate concentrated to dryness. The residue was purified by column chromatography on silica gel (PE/EA=3/1) to give (S)-6-chloro-N-(oxetan-2-ylmethyl)-4-(phenylsulfonyl)pyridazin-3-amine (4.3 g, yield: 73%) as yellow solid. MS Calcd.: 339.04; MS Found: 340.0 [M+H]⁺.

### Step C: (S)-4-azido-6-chloro-N-(oxetan-2-ylmethyl)pyridazin-3-amine

To a mixture of (S)-6-chloro-N-(oxetan-2-ylmethyl)-4-(phenylsulfonyl)pyridazin-3-amine (4.3 g, 12.7 mmol) in DMSO (70 mL) was added NaN₃ (3.3 g, 50.7 mmol) and the mixture stirred at 70° C. for 2 hours. After the reaction was completed, the mixture was quenched with H₂O (100 mL) and extracted with EA (200 mL*2). The extracts were wash with brine (200 mL*3). The organic layer was dried over anhydrous sodium sulfate, filtered, and concentrated to dryness. The residue was purified by column chromatography on silica gel (PE/EA=1/1) to give (S)-4-azido-6-chloro-N-(oxetan-2-ylmethyl)pyridazin-3-amine (2.3 g, yield: 74%) as brown oil. MS Calcd.: 240.0; MS Found: 241.2 [M+H]⁺.

### Step D: (S)-6-chloro-N3-(oxetan-2-ylmethyl)pyridazine-3,4-diamine

To a solution of (S)-4-azido-6-chloro-N-(oxetan-2-ylmethyl)pyridazin-3-amine (2.2 g, 10.3 mmol) in THE (40 mL) was added Pd/C (440 mg, 10% on Carbon, wetted with ca. 55% water). The mixture was stirred at rt for 2 hours under H₂ (1 atm). The reaction mixture was filtered through a celite pad. The filtrate was concentrated to give (S)-6-chloro-N3-(oxetan-2-ylmethyl)pyridazine-3,4-diamine (2.0 g crude) as a brown solid. MS Calcd.: 214.1; MS Found: 215.1 [M+H]⁺.

### Step E: (S)-3-chloro-6-(chloromethyl)-7-(oxetan-2-ylmethyl)-7H-imidazo[4,5-c]pyridazine

To a mixture of (S)-6-chloro-N3-(oxetan-2-ylmethyl)pyridazine-3,4-diamine (2.0 g) in THE (50 mL) was added 2-chloroacetic anhydride (1.6 g, 9.3 mmol). The mixture was stirred at rt for 1 h and then heated to 60° C. for 7 hours. The reaction mixture was concentrated, and the residue purified by column chromatography on silica gel (PE/EA=1/1) to give (S)-3-chloro-6-(chloromethyl)-7-(oxetan-2-ylmethyl)-7H-imidazo[4,5-c]pyridazine (1.1 g, yield: 43%) as yellow oil. MS Calcd.: 272.0; MS Found: 272.9 [M+H]⁺.

### Step F: (S)-3-chloro-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine

To a mixture of (S)-3-chloro-6-(chloromethyl)-7-(oxetan-2-ylmethyl)-7H-imidazo[4,5-c]pyridazine (300 mg, 1.1 mmol) in DMF (6 mL) was added 2-(4-chloro-2-fluorobenzyl)-5-fluoro-4-(piperidin-4-yloxy)pyrimidine (530 mg, TFA salt, 1.2 mmol) and K₂CO₃ (455 mg, 3.3 mmol). The mixture was stirred at 60° C. for 2 hours. The reaction was quenched with water (20 mL), extracted with ethyl acetate (30 mL×2). The organic layers were combined, dried over Na₂SO₄, filtered, and concentrated under vacuum. The residue was purified by silica gel chromatography, eluting with DCM:MeOH=30:1 to give (S)-3-chloro-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine (500 mg, yield: 79%) as a brown solid. MS Calcd.: 575.1; MS Found: 576.0 [M+H]⁺.

### Step G: ethyl (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxylate

To a mixture of (S)-3-chloro-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine (450 mg, 0.78 mmol) in EtOH (8 mL) was added Pd(dppf)Cl₂ (114.5 mg, 0.16 mmol) and KOAc (230 mg, 2.3 mmol). The mixture was stirred at 75° C. for 4 hours under an atmosphere of CO (balloon). The solvent was removed in vacuo. The residue was purified by silica gel chromatography, eluting with DCM:MeOH=20:1 to give ethyl (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxylate (460 mg, yield: 96%) as a brown solid. MS Calcd.: 613.2; MS Found: 614.2 [M+H]⁺.

### Step H: (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxylic acid

To a mixture of ethyl (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxylate (460 mg, 0.75 mmol) in MeOH (6 mL) was added aqueous NaOH (2 M, 2 mL). The mixture was stirred at rt for 2 hours. After the reaction was completed, the pH value was adjusted to 5-6 with 1 N HCl (aq.) and extracted with ethyl acetate (15 mL×2). The combined organic layer was dried over anhydrous sodium sulfate, filtered, and evaporated to give (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxylic acid (390 mg, yield: 89%) as an off-white solid. MS Calcd.: 585.2; MS Found: 586.2 [M+H]⁺.

### Step I: (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxamide

A mixture of (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxylic acid (370 mg), NH₄Cl (50 mg, 0.95 mmol), HATU (360 mg, 0.95 mmol) and DIEA (163 mg, 1.3 mmol) in DMF (6 mL) was stirred at rt for 2 hours. After the reaction was completed, the mixture was diluted with EA (15 mL), washed with H₂O (10 mL×2). The organic layer was dried over anhydrous sodium sulfate, filtered, and evaporated to dryness. The residue was purified by silica gel chromatography, eluting with DCM:MeOH=20:1 to give (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxamide (250 mg, yield: 68%) as a brown solid. MS Calcd.: 584.2; MS Found: 585.2 [M+H]⁺.

### Step J: (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carbonitrile

To a solution of (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carboxamide (230 mg, 0.39 mmol) in DCM (4 mL) was added TEA (158 mg, 1.57 mmol) and TFAA (248 mg, 1.18 mmol). The solution was stirred at room temperature for 3 hours. After the reaction was completed, the mixture was diluted with EA (30 mL), washed with H₂O (15 mL×2). The combined organic layer was dried over anhydrous sodium sulfate, filtered, and evaporated to dryness. The residue was purified by silica gel chromatography, eluting with DCM:MeOH=20:1 to give (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carbonitrile (140 mg, yield: 63%) as a brown solid. MS Calcd.: 566.2; MS Found: 567.2 [M+H]⁺.

### Step K: (S)-6-((4-((2-(4-chloro-2-fluorobenzyl)-5-fluoropyrimidin-4-yl)oxy)piperidin-1-yl)methyl)-N′-hydroxy-7-(oxetan-2-ylmethyl)-7H-imidazo[4,5-c]pyridazine-3-carboximidamide

A solution of (S)-6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazine-3-carbonitrile (140 mg, 0.25 mmol), HONH₂·HCl (35 mg, 0.49 mmol) and TEA (75 mg, 0.74 mmol) in EtOH (3 mL) was stirred at 90° C. for 1 h. After the reaction was completed, the solid precipitated was collected, dried to give (S)-6-((4-((2-(4-chloro-2-fluorobenzyl)-5-fluoropyrimidin-4-yl)oxy)piperidin-1-yl)methyl)-N′-hydroxy-7-(oxetan-2-ylmethyl)-7H-imidazo[4,5-c]pyridazine-3-carboximidamide (90 mg, yield: 61%) as a white solid. MS Calcd.: 599.2; MS Found: 600.2 [M+H]⁺.

### Step L: (S)-3-(6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazin-3-yl)-5-(trifluoromethyl)-1,2,4-oxadiazole

To a solution of (S)-6-((4-((2-(4-chloro-2-fluorobenzyl)-5-fluoropyrimidin-4-yl)oxy)piperidin-1-yl)methyl)-N′-hydroxy-7-(oxetan-2-ylmethyl)-7H-imidazo[4,5-c]pyridazine-3-carboximidamide (90 mg) in THE (3 mL) was added TFAA (126 mg, 0.6 mmol) and the mixture stirred at rt for 2 hours. After the reaction was completed, the reaction was quenched with sodium bicarbonate aqueous solution (10 mL), extracted with ethyl acetate (15 mL×3). The organic layers were combined, dried over Na₂SO₄, filtered and concentrated under vacuum to give (S)-3-(6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazin-3-yl)-5-(trifluoromethyl)-1,2,4-oxadiazole (74 mg, 85% purity, yield: 62%) as a light yellow solid. MS Calcd.: 677.2; MS Found: 678.2 [M+H]⁺.

### Step M: 2-[(4-chloro-2-fluorophenyl)methyl]-5-fluoro-4-({1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}oxy)pyrimidine (Compound 180a)

A mixture of (S)-3-(6-((4-((2-(4-chloro-2-fluorobenzyl)-5-fluoropyrimidin-4-yl)oxy)piperidin-1-yl)methyl)-7-(oxetan-2-ylmethyl)-7H-imidazo[4,5-c]pyridazin-3-yl)-5-(trifluoromethyl)-1,2,4-oxadiazole (74 mg, 0.11 mmol) and N₂H₄·H₂O (22 mg, 0.44 mmol) in DMF (1.5 mL) was stirred at rt for 2 hours. After the reaction was completed, the mixture was purified directly by Prep-HPLC (0.1% FA/H₂O/CH₃CN) to give 2-[(4-chloro-2-fluorophenyl)methyl]-5-fluoro-4-({1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}oxy)pyrimidine (Compound 180a) (40 mg, yield: 54%) as a white solid.

MS Calcd.: 676.2; MS Found: 677.0 [M+H]⁺.

¹H NMR (400 MHz, DMSO-d6): δ 8.48-8.53 (m, 2H), 7.36-7.44 (m, 2H), 7.22-7.27 (m, 1H), 5.20-5.30 (m, 1H), 4.95-5.04 (m, 2H), 4.83-4.92 (m, 1H), 4.48-4.56 (m, 1H), 4.38-4.44 (m, 1H), 4.15 (s, 2H), 4.05 (s, 2H), 2.70-2.87 (m, 3H), 2.32-2.60 (m, 3H), 1.89-2.01 (m, 2H), 1.65-1.78 (m, 2H). ¹⁹F-NMR (377 MHz): −63.17, −114.13, −157.36.

### Example 66: 2-[(4-chloro-2-fluorophenyl)methyl]-6-({1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}oxy)pyridine (Compound 181a)

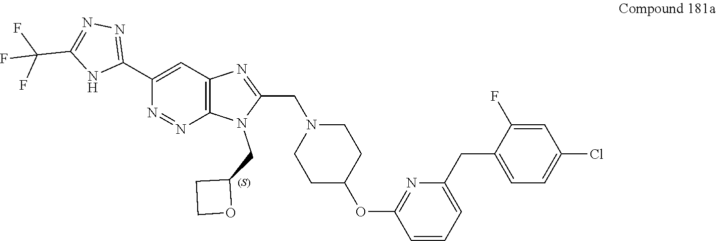

2-(4-chloro-2-fluorobenzyl)-6-(piperidin-4-yloxy)pyridine. 2-[(4-chloro-2-fluorophenyl)methyl]-6-({1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}oxy)pyridine Compound 181a (41 mg) was obtained as a white solid.

MS Calcd.: 657.2; MS Found: 658.0 [M+H]⁺.

¹H NMR (400 MHz, DMSO-d6): δ 8.50 (s, 1H), 7.58-7.63 (m, 1H), 7.35-7.40 (m, 2H), 7.23 (dd, J=8.4 Hz, 2.0 Hz, 1H), 6.82 (d, J=7.2 Hz, 1H), 6.59 (d, J=8.0 Hz, 1H), 5.21-5.30 (m, 1H), 4.98-5.05 (m, 1H), 4.79-4.92 (m, 2H), 4.49-4.56 (m, 1H), 4.37-4.45 (m, 1H), 4.04 (s, 2H), 4.01 (s, 2H), 2.70-2.85 (m, 3H), 2.50-2.60 (m, 1H), 2.32-2.40 (m, 2H), 1.83-1.94 (m, 2H), 1.53-1.67 (m, 2H). ¹⁹F-NMR (377 MHz): −63.31, −114.57.

### Example 67: 2-[(4-chloro-2-fluorophenyl)methyl]-4-({1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}oxy)pyrimidine (Compound 182a)

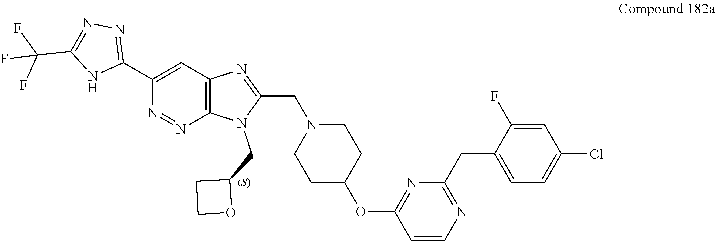

2-(4-chloro-2-fluorobenzyl)-4-(piperidin-4-yloxy)pyrimidine. 2-[(4-chloro-2-fluorophenyl)methyl]-4-({1-[(7-{[(2S)-oxetan-2-yl]methyl}-3-[5-(trifluoromethyl)-4H-1,2,4-triazol-3-yl]-7H-imidazo[4,5-c]pyridazin-6-yl)methyl]piperidin-4-yl}oxy)pyrimidine Compound 182a (27 mg) was obtained as a white solid.

MS Calcd.: 658.2; MS Found: 659.0 [M+H]⁺.

¹H NMR (400 MHz, DMSO-d6): δ 8.52 (s, 1H), 8.41 (d, J=5.6 Hz, 1H), 7.36-7.43 (m, 2H), 7.24 (dd, J=8.0 Hz, 1.6 Hz, 1H), 6.74 (d, J=5.6 Hz, 1H), 5.21-5.30 (m, 1H), 4.97-5.05 (m, 1H), 4.85-4.95 (m, 2H), 4.47-4.55 (m, 1H), 4.37-4.44 (m, 1H), 4.15 (s, 2H), 4.05 (s, 2H), 2.70-2.85 (m, 3H), 2.48-2.60 (m, 1H), 2.31-2.42 (m, 2H), 1.86-1.96 (m, 2H), 1.59-1.71 (m, 2H). ¹⁹F-NMR (377 MHz): −63.62, −114.06.

### Example 68: 2-[(4-chloro-2-fluorophenyl)methyl]-6-{[1-({6-[5-(methoxymethyl)-4H-1,2,4-triazol-3-yl]-3-{[(2S)-oxetan-2-yl]methyl}-3H-imidazo[4,5-c]pyridin-2-yl}methyl)piperidin-4-yl]oxy}pyridine (Compound 183a)

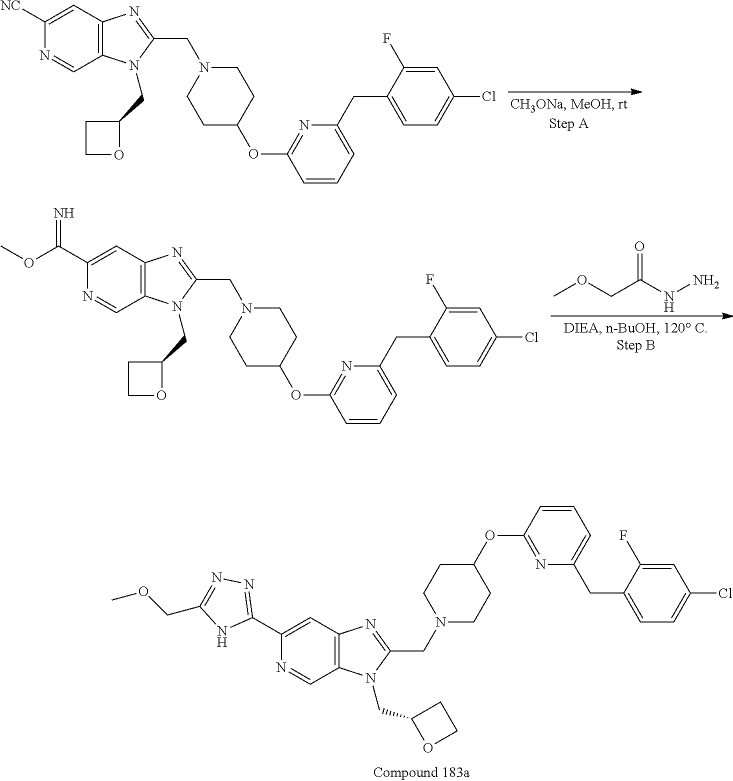

### Step A: methyl (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbimidate

A mixture of (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbonitrile (300 mg, 0.55 mmol), Sodium methoxide (297 mg, 5.5 mmol) in MeOH (5.0 mL) was stirred at room temperature for 1 hour. After the reaction was completed, the mixture was concentrated in vacuum. The residue was purified by column chromatography on silica gel (DCM/MeOH=40/1) to give methyl (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbimidate (166 mg, yield: 52%) as yellow oil. MS Calcd.: 578.2; MS Found: 579.2 [M+H]⁺.

### Step B: 2-[(4-chloro-2-fluorophenyl)methyl]-6-{[1-({6-[5-(methoxymethyl)-4H-1,2,4-triazol-3-yl]-3-{[(2S)-oxetan-2-yl]methyl}-3H-imidazo[4,5-c]pyridin-2-yl}methyl)piperidin-4-yl]oxy}pyridine (Compound 183a)

A mixture of methyl (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbimidate (80 mg, 0.14 mmol), 2-methoxyacetohydrazide (29 mg, 0.28 mmol), DIEA (54 mg, 0.42 mmol) in n-BuOH (2.0 mL) was stirred at 120° C. for 12 hours. After the reaction was completed, the reaction mixture was concentrated and the residue purified by Prep-HPLC (0.1% TFA/H₂O/CH₃CN) to give 2-[(4-chloro-2-fluorophenyl)methyl]-6-{[1-({6-[5-(methoxymethyl)-4H-1,2,4-triazol-3-yl]-3-{[(2S)-oxetan-2-yl]methyl}-3H-imidazo[4,5-c]pyridin-2-yl}methyl)piperidin-4-yl]oxy}pyridine Compound 183a (30 mg, yield: 34%) as a white solid.

MS Calcd.: 632.2; MS Found: 633.1 [M+H]⁺.

¹H NMR (400 MHz, DMSO-d₆): 9.24 (s, 1H), 8.34 (s, 1H), 7.67 (t, J=7.6 Hz, 1H), 7.35-7.42 (m, 2H), 7.24 (dd, J=8.4 Hz, 2.0 Hz, 1H), 6.88 (d, J=7.2 Hz, 1H), 6.67 (d, J=8.4 Hz, 1H), 5.05-5.16 (m, 2H), 4.84-4.95 (m, 3H), 4.70-4.77 (m, 1H), 4.47-4.56 (m, 3H), 4.36-4.44 (m, 1H), 4.02 (s, 3H), 2.65-2.85 (m, 3H), 2.30-2.45 (m, 1H), 2.05-2.24 (m, 6H), 1.85-2.05 (m, 2H). ¹⁹F-NMR (377 MHz): −114.52.

### Example 69: 2-[(4,4-difluoropiperidin-1-yl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 184a)

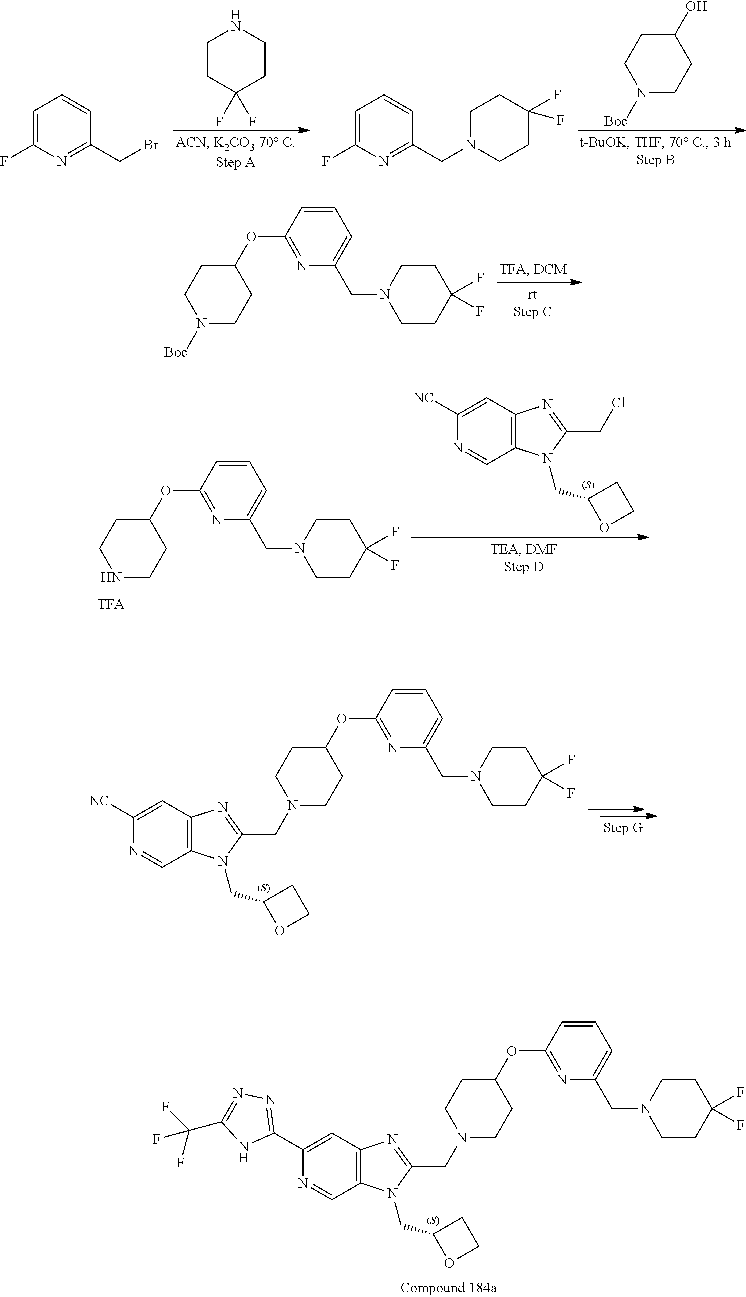

### Step A: 2-((4,4-difluoropiperidin-1-yl)methyl)-6-fluoropyridine

A mixture of 2-(bromomethyl)-6-fluoropyridine (700 mg, 3.7 mmol), 4,4-difluoropiperidine (446 mg, 3.7 mmol) and K₂CO₃ in ACN (5 mL) The reaction was stirred at 70° C. for 4 hours. After the reaction was completed, the reaction was quenched with H₂O (20 mL) and extracted with ethyl acetate (25 mL×3). The organic layer was combined and washed with brine (20 mL×2), dried over sodium sulfate, filtered and concentrated in vacuum. The crude product was purified by column chromatography (PE:EA=5:1) to give 2-((4,4-difluoropiperidin-1-yl)methyl)-6-fluoropyridine (374 mg, 57% yield) as a yellow oil. MS Calcd.: 230.1; MS Found: 231.1 [M+H]⁺.

### Step B: tert-butyl 4-((6-((4,4-difluoropiperidin-1-yl)methyl)pyridin-2-yl)oxy)piperidine-1-carboxylate

A mixture of 2-((4,4-difluoropiperidin-1-yl)methyl)-6-fluoropyridine (520 mg, 2.3 mmol) and tert-butyl 4-hydroxypiperidine-1-carboxylate (1.4 g, 6.8 mmol) in THE (5 mL) was added t-BuOK stirred at 70° C. for 3 hours. After the reaction was completed, the reaction was quenched with H₂O (20 mL) and extracted with ethyl acetate (25 mL×3). The organic layer was combined and washed with brine (20 mL×2), dried over sodium sulfate, filtered, and concentrated in vacuum. The reaction was purified by column chromatography (PE:EA=5:1) to give tert-butyl 4-((6-((4,4-difluoropiperidin-1-yl)methyl)pyridin-2-yl)oxy)piperidine-1-carboxylate (496 mg, 53% yield) as yellow oil. MS Calcd.: 411.2; MS Found: 412.2 [M+H]⁺.

### Step C: 2-((4,4-difluoropiperidin-1-yl)methyl)-6-(piperidin-4-yloxy)pyridine TFA salt

A mixture of tert-butyl 4-((6-((4,4-difluoropiperidin-1-yl)methyl)pyridin-2-yl)oxy)piperidine-1-carboxylate (496 mg, 1.21 mmol) in DCM (3 mL) was added TFA (1 mL). The reaction was stirred at room temperature for 1 hour. After the reaction was completed, the reaction was concentrated in vacuum to give 2-((4,4-difluoropiperidin-1-yl)methyl)-6-(piperidin-4-yloxy)pyridine TFA salt (420 mg, crude) as yellow oil. MS Calcd.: 311.2; MS Found: 312.2 [M+H]⁺.

### Step D: (S)-2-((4-((6-((4,4-difluoropiperidin-1-yl)methyl)pyridin-2-yl)oxy)piperidin-1-yl)methyl)-3-(oxetan-2-ylmethyl)-3H-imidazo[4,5-c]pyridine-6-carbonitrile

2-((4,4-difluoropiperidin-1-yl)methyl)-6-(piperidin-4-yloxy)pyridine TFA salt (181 mg), (S)-2-(chloromethyl)-3-(oxetan-2-ylmethyl)-3H-imidazo[4,5-c]pyridine-6-carbonitrile (100 mg, 0.38 mmol) and TEA (2 mL) in DMF (2 mL) was stirred at 50° C. for 2 hours. After the reaction was completed, the reaction was quenched with H₂O (20 mL) and extracted with ethyl acetate (25 mL×3). The organic layer was combined and washed with brine (20 mL×2), dried over sodium sulfate, filtered and concentrated in vacuum. The reaction was purified by column chromatography EA to give (S)-2-((4-((6-(4-chloro-3-fluorobenzyl)pyridin-2-yl)oxy)piperidin-1-yl)methyl)-3-(oxetan-2-ylmethyl)-3H-imidazo[4,5-c]pyridine-6-carbonitrile (139 mg, 44% yield) as yellow oil. MS Calcd.: 537.3; MS Found: 538.2 [M+H]⁺.

### Step G: 2-[(4,4-difluoropiperidin-1-yl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 184a)

2-[(4,4-difluoropiperidin-1-yl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 184a) (21 mg) was obtained with the similar procedure of Compound 149a as a white solid.

MS Calcd.: 647.3; MS Found: 648.4 [M+H]⁺.

¹H NMR (400 MHz, DMSO-d₆): 9.16 (s, 1H), 8.28 (s, 1H), 7.65 (t, J=8.0 Hz, 1H), 6.98 (d, J=7.6 Hz, 1H), 6.64 (d, J=8.0 Hz, 1H), 5.11-5.20 (m, 1H), 4.97-5.06 (m, 1H), 4.87-8.97 (m, 1H), 4.70-4.79 (m, 1H), 4.47-4.56 (m, 1H), 4.38-4.46 (m, 1H), 4.01 (d, J=13.6 Hz, 1H), 3.87 (d, J=13.6 Hz, 1H), 3.59 (s, 2H), 2.67-2.87 (m, 3H), 2.54-2.60 (m, 4H), 2.31-2.51 (m, 3H), 1.89-2.03 (m, 6H), 1.58-1.72 (m, 2H).

### Example 70: 2-[(4-chloro-3-fluorophenyl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 185a)

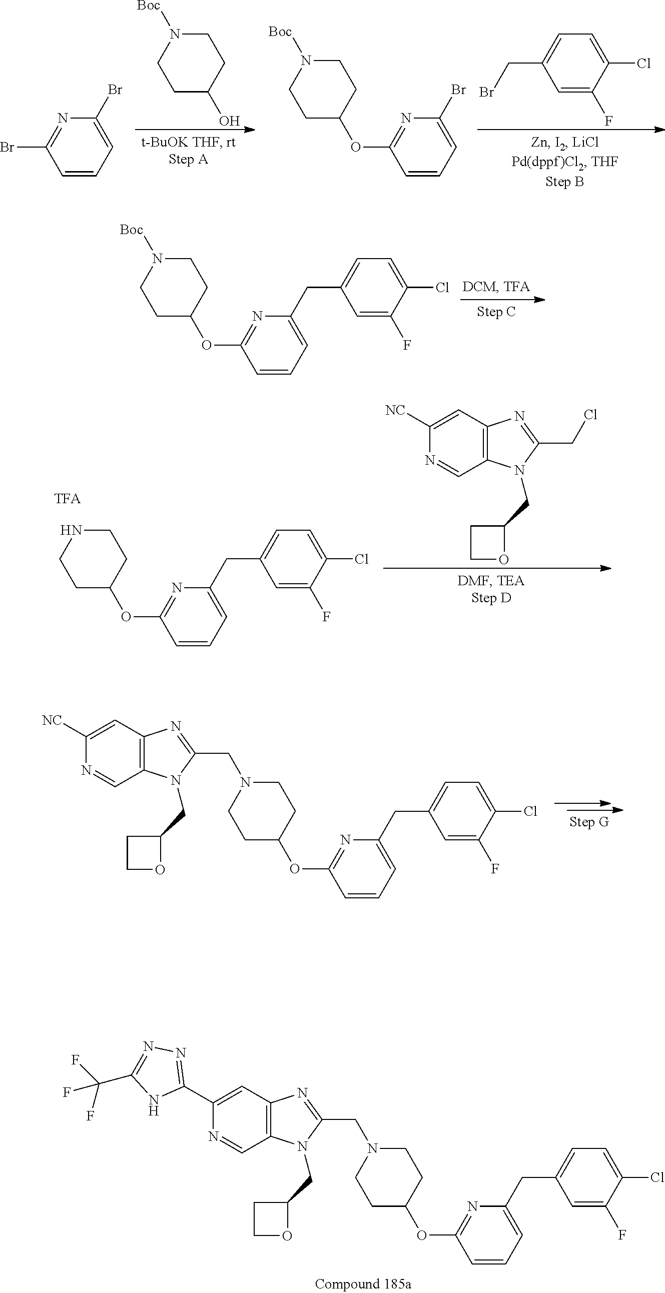

### Step A: tert-butyl 4-((6-bromopyridin-2-yl)oxy)piperidine-1-carboxylate

To a solution of 2,6-dibromopyridine (10.0 g, 42.2 mmol) and tert-butyl 4-hydroxypiperidine-1-carboxylate (2.0 g, 42.2 mmol) in THE (100 mL) was added t-BuOK in THF (50.6 mL, 1.0 mol/L, 50.6 mmol) at 0° C. The reaction was stirred at room temperature for 1 hour. After the reaction was completed, the reaction was quenched with H₂O (100 mL) and extracted with ethyl acetate (200 mL×3). The organic layer was combined and washed with brine (100 mL×2), dried over sodium sulfate, filtered, and concentrated in vacuum. The crude product was purified by column chromatography (PE:EA=5:1) to give tert-butyl 4-((6-bromopyridin-2-yl)oxy)piperidine-1-carboxylate (15.2 g, 100% yield) as colorless oil. MS Calcd.: 356.1; MS Found: 301.0 [M−56+H]⁺.

### Step B: tert-butyl 4-((6-(4-chloro-3-fluorobenzyl)pyridin-2-yl)oxy)piperidine-1-carboxylate

To a solution of 4-(bromomethyl)-1-chloro-2-fluorobenzene (500 mg, 2.2 mmol) in THE (5 mL) was added Zn (437 mg, 6.6 mmol), LiCl (47 mg, 1.1 mmol) and I₂ (57 mg, 0.22 mmol) at room temperature. The reaction was degassed with N₂ and stirred at 50° C. for 0.5 hour. Then tert-butyl 4-((6-bromopyridin-2-yl)oxy)piperidine-1-carboxylate (319 mg, 0.90 mmol) and Pd(dppf)Cl₂ (164 mg, 0.22 mmol) was added. The reaction was stirred at 70° C. for 1 hour. After the reaction was completed, the reaction was filtered and concentrated in vacuum. The residue was purified by column chromatography (PE:EA=5:1) to give tert-butyl 4-((6-(4-chloro-3-fluorobenzyl)pyridin-2-yl)oxy)piperidine-1-carboxylate (230 mg, 61% yield) as yellow oil. MS Calcd.: 420.2; MS Found: 421.0 [M+H]⁺.

### Step C: of 2-(4-chloro-3-fluorobenzyl)-6-(piperidin-4-yloxy)pyridine TFA salt

To a solution of tert-butyl 4-((6-(4-chloro-3-fluorobenzyl)pyridin-2-yl)oxy)piperidine-1-carboxylate (130 g, 0.24 mmol) in DCM (3 mL) was added TFA (1 mL). The reaction was stirred at room temperature for 1 hour. After the reaction was completed, the reaction was concentrated in vacuum to give 2-(4-chloro-3-fluorobenzyl)-6-(piperidin-4-yloxy)pyridine TFA salt (100 mg, crude) as yellow oil.

MS Calcd.: 320.1; MS Found: 321.1 [M+H]⁺.

### Step D: (S)-2-((4-((6-(4-chloro-3-fluorobenzyl)pyridin-2-yl)oxy)piperidin-1-yl)methyl)-3-(oxetan-2-ylmethyl)-3H-imidazo[4,5-c]pyridine-6-carbonitrile

A mixture of 2-(4-chloro-3-fluorobenzyl)-6-(piperidin-4-yloxy)pyridine TFA salt (100 mg), (S)-2-(chloromethyl)-3-(oxetan-2-ylmethyl)-3H-imidazo[4,5-c]pyridine-6-carbonitrile (60 mg, 0.23 mmol) and TEA (70 mg, 0.69 mmol) in DMF (2 mL) was stirred at 60° C. for 2 hours. After the reaction was completed, the reaction mixture was concentrated in vacuum. The residue was purified by column chromatography (PE:EA=1:1) to give (S)-2-((4-((6-(4-chloro-3-fluorobenzyl)pyridin-2-yl)oxy)piperidin-1-yl)methyl)-3-(oxetan-2-ylmethyl)-3H-imidazo[4,5-c]pyridine-6-carbonitrile (80 mg, 63% yield) as yellow oil. MS Calcd.: 546.2; MS Found: 547.1 [M+H]⁺.

### Step G: 2-[(4-chloro-3-fluorophenyl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine

2-[(4-chloro-3-fluorophenyl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 185a) (11 mg) was obtained with the similar procedure of Compound 149a as a white solid.

MS Calcd.: 656.2; MS Found: 657.3 [M+H]⁺.

¹H NMR (400 MHz, DMSO-d₆): δ 9.16 (s, 1H), 8.28 (s, 1H), 7.60 (t, J=7.6 Hz, 1H), 7.49 (t, J=8.0 Hz, 1H), 7.36 (d, J=10.0 Hz, 1H), 7.15 (d, J=8.0 Hz, 1H), 6.86 (d, J=7.2 Hz, 1H), 6.59 (d, J=8.4 Hz, 1H), 5.12-5.20 (m, 1H), 4.88-5.00 (m, 2H), 4.71-4.79 (m, 1H), 4.49-4.56 (m, 1H), 4.38-4.48 (m, 1H), 3.95-4.05 (m, 3H), 3.86 (d, J=14.0 Hz, 1H), 2.66-2.88 (m, 3H), 2.40-2.51 (m, 1H), 2.31-2.40 (m, 2H), 1.88-1.97 (m, 2H), 1.51-1.68 (m, 2H). ¹⁹F-NMR (377 MHz): −63.68, −116.99.

### Example 71: 2-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)-6-{[4-(trifluoromethyl)-1H-pyrazol-1-yl]methyl}pyridine (Compound 186a)

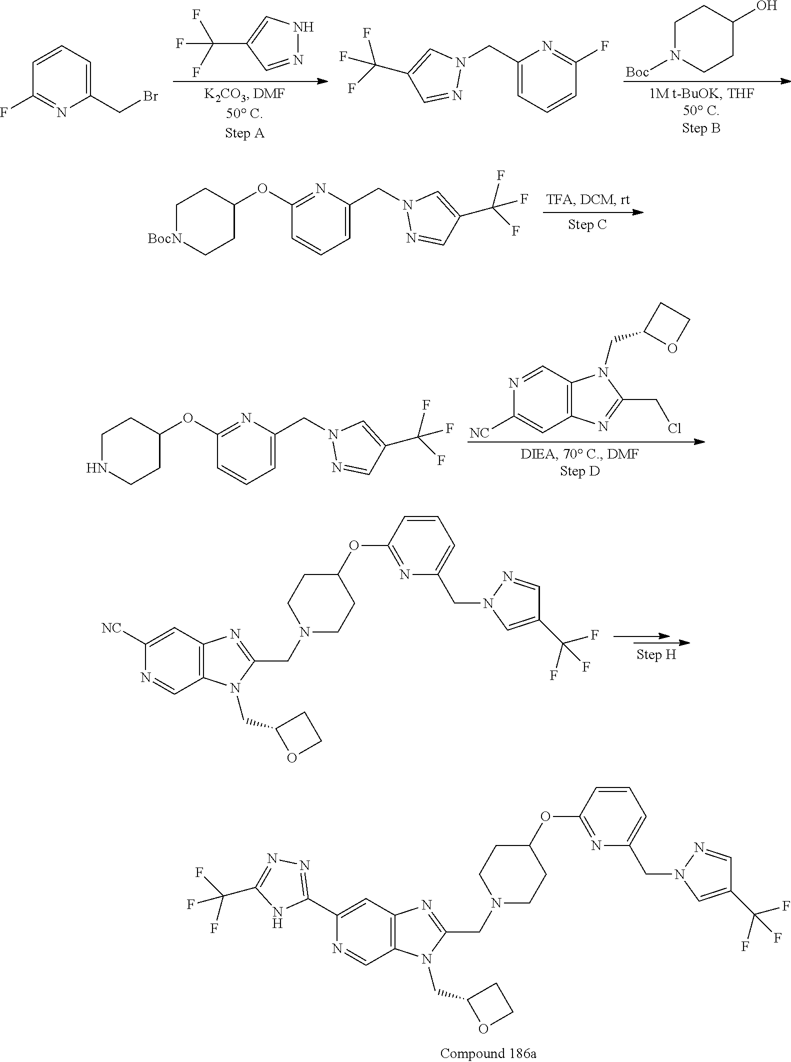

### Step A: 2-fluoro-6-((4-(trifluoromethyl)-1H-pyrazol-1-yl)methyl)pyridine

A mixture of 2-(bromomethyl)-6-fluoropyridine (900 mg, 4.7 mmol), 4-(trifluoromethyl)-1H-pyrazole (709 mg, 5.2 mmol), K₂CO₃ (1307 mg, 9.5 mmol) in DMF (15 mL) was stirred at 50° C. for 2 hours. After the reaction was completed, the mixture was diluted with EA (60 mL), washed with H₂O (30 mL*2). The combined organic layer was dried over anhydrous sodium sulfate, filtered, and evaporated to dryness. The residue was purified by column chromatography on silica gel (PE/EA=10/1) to give 2-fluoro-6-((4-(trifluoromethyl)-1H-pyrazol-1-yl)methyl)pyridine (713 mg, yield: 61%) as a yellow oil. MS Calcd.: 245.1; MS Found: 246.0 [M+H]⁺.

### Step B: tert-butyl 4-((6-((4-(trifluoromethyl)-1H-pyrazol-1-yl)methyl)pyridin-2-yl)oxy)piperidine-1-carboxylate

To a solution of 2-fluoro-6-((4-(trifluoromethyl)-1H-pyrazol-1-yl)methyl)pyridine (713 mg, 2.9 mmol), tert-butyl 4-hydroxypiperidine-1-carboxylate (1170 mg, 5.8 mmol) in DMF (15 mL) was added t-BuOK (4.4 mL, 1 mol/L in THF) at 0° C. The solution was stirred at 50° C. for 2 hours. After the reaction was completed, the mixture was diluted with EA (60 mL), washed with H₂O (30 mL*2). The combined organic layer was dried over anhydrous sodium sulfate, filtered, and evaporated to dryness. The residue was purified by column chromatography on silica gel (PE/EA=10/1) to give tert-butyl 4-((6-((4-(trifluoromethyl)-1H-pyrazol-1-yl)methyl)pyridin-2-yl)oxy)piperidine-1-carboxylate (1.0 g, yield: 81%) as a yellow solid. MS Calcd.: 426.2; MS Found: 449.1 [M+23]⁺.

### Step C: 2-(piperidin-4-yloxy)-6-((4-(trifluoromethyl)-1H-pyrazol-1-yl)methyl)pyridine

To a solution of tert-butyl 4-((6-((4-(trifluoromethyl)-1H-pyrazol-1-yl)methyl)pyridin-2-yl)oxy)piperidine-1-carboxylate (127 mg, 0.3 mmol) in DCM (2 mL) was added TFA (0.5 mL). The solution was stirred at rt for 1 hour. The solvent was removed in vacuo to give 2-(piperidin-4-yloxy)-6-((4-(trifluoromethyl)-1H-pyrazol-1-yl)methyl)pyridine TFA salt (125 mg, crude) as a yellow oil. MS Calcd.: 326.1; MS Found: 327.1 [M+H]⁺.

### Step D: (S)-3-(oxetan-2-ylmethyl)-2-((4-((6-((4-(trifluoromethyl)-1H-pyrazol-1-yl)methyl)pyridin-2-yl)oxy)piperidin-1-yl)methyl)-3H-imidazo[4,5-c]pyridine-6-carbonitrile

A mixture of 2-(piperidin-4-yloxy)-6-((4-(trifluoromethyl)-1H-pyrazol-1-yl)methyl)pyridine TFA salt (125 mg), (S)-2-(chloromethyl)-3-(oxetan-2-ylmethyl)-3H-imidazo[4,5-c]pyridine-6-carbonitrile (60 mg, 0.23 mmol), DIEA (295 mg, 2.3 mmol) in DMF (2 mL) was stirred at 70° C. for 2 hours. After the reaction was completed, the mixture was diluted with EA (60 mL), washed with H₂O (30 mL*2). The combined organic layer was dried over anhydrous sodium sulfate, filtered, and evaporated to dryness. The residue was purified by column chromatography on silica gel (PE/EA=1/1) to give (S)-3-(oxetan-2-ylmethyl)-2-((4-((6-((4-(trifluoromethyl)-1H-pyrazol-1-yl)methyl)pyridin-2-yl)oxy)piperidin-1-yl)methyl)-3H-imidazo[4,5-c]pyridine-6-carbonitrile (95 mg, yield: 75%) as a yellow oil. MS Calcd.: 552.2; MS Found: 553.2 [M+H]⁺.

### Step H: 2-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)-6-{[4-(trifluoromethyl)-1H-pyrazol-1-yl]methyl}pyridine (Compound 186a)

2-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)-6-{[4-(trifluoromethyl)-1H-pyrazol-1-yl]methyl}pyridine Compound 186a (20 mg) was obtained with the similar procedure of Compound 149a as a white solid.

MS Calcd.: 662.2; MS Found: 663.1 [M+H]⁺.

¹H NMR (400 MHz, DMSO-d6): δ 9.15 (s, 1H), 8.53 (s, 1H), 8.27 (s, 1H), 7.90 (s, 1H), 7.65-7.71 (m, 1H), 6.80 (d, J=7.2 Hz, 1H), 6.69 (d, J=8.0 Hz, 1H), 5.41 (s, 2H), 5.11-5.19 (m, 1H), 4.78-4.95 (m, 2H), 4.69-4.78 (m, 1H), 4.48-4.56 (m, 1H), 4.37-4.47 (m, 1H), 3.98 (d, J=13.6 Hz, 1H), 3.84 (d, J=13.6 Hz, 1H), 2.66-2.85 (m, 3H), 2.40-2.53 (m, 1H), 2.25-2.35 (m, 2H), 1.82-1.92 (m, 2H), 1.50-1.63 (m, 2H). ¹⁹F-NMR (377 MHz): −54.31, −63.52.

### Example 72: 2-(2-{[4-({6-[(4-chloro-2-fluorophenyl)methyl]pyridin-2-yl}oxy)piperidin-1-yl]methyl}-3-{[(2S)-oxetan-2-yl]methyl}-3H-imidazo[4,5-c]pyridin-6-yl)-6-methoxypyrazine (Compound 187a)

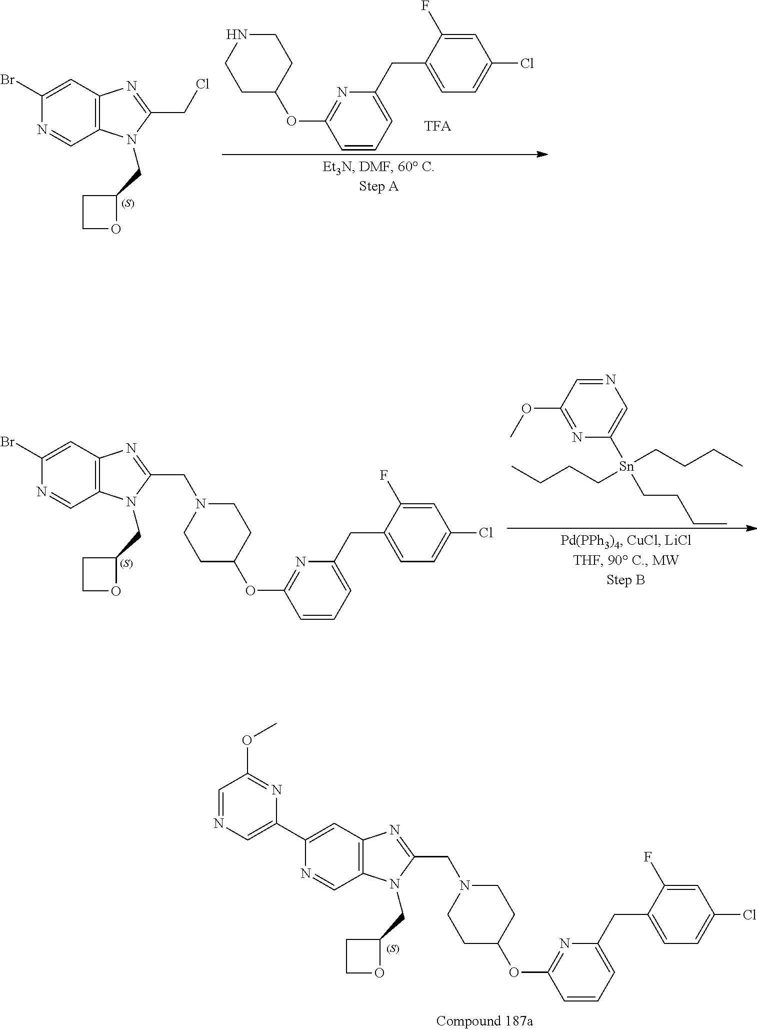

### Step A: (S)-6-bromo-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine

A mixture of (S)-6-bromo-2-(chloromethyl)-3-(oxetan-2-ylmethyl)-3H-imidazo[4,5-c]pyridine (400 mg, 1.27 mmol), 2-(4-chloro-2-fluorobenzyl)-6-(piperidin-4-yloxy)pyridine TFA salt (crude 815 mg, 1.9 mmol), Et₃N (384 mg, 3.8 mmol) in DMF (4 mL) was stirred at 60° C. for 4 h. After the reaction was completed, the mixture was diluted with EA (50 mL), washed with water (40 mL*3). The organic layer was dried over anhydrous sodium sulfate, filtered, and evaporated to dryness. The residue was purified by column chromatography on silica gel (PE/EA=2/1) to give (S)-6-bromo-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine (300 mg, yield: 39%) as yellow oil. MS Calcd.: 599.1; MS Found: 600.1 [M+H]⁺.

### Step B: 2-(2-{[4-({6-[(4-chloro-2-fluorophenyl)methyl]pyridin-2-yl}oxy)piperidin-1-yl]methyl}-3-{[(2S)-oxetan-2-yl]methyl}-3H-imidazo[4,5-c]pyridin-6-yl)-6-methoxypyrazine (Compound 187a)

A mixture of (S)-6-bromo-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine (100 mg, 0.16 mmol), 2-methoxy-6-(tributylstannyl)pyrazine (200 mg, 0.50 mmol), Pd(PPh₃)₄ (18 mg, 0.016 mmol), CuCl (4.2 mg, 0.032 mmol), LiCl (6.8 mg, 0.16 mmol) in THE (2 mL) was stirred under microwave radiation for 4 hours at 90° C. After the reaction was completed, the mixture was filtered and the filtrate was purified by Prep-HPLC to afford 2-(2-{[4-({6-[(4-chloro-2-fluorophenyl)methyl]pyridin-2-yl}oxy)piperidin-1-yl]methyl}-3-{[(2S)-oxetan-2-yl]methyl}-3H-imidazo[4,5-c]pyridin-6-yl)-6-methoxypyrazine Compound 187a (24 mg, yield: 23%) as a white solid.

MS Calcd.: 629.2; MS Found: 630.1 [M+H]⁺.

¹H NMR (400 MHz, DMSO-d₆): δ 9.12 (s, 2H), 8.56 (s, 1H), 8.30 (s, 1H), 7.60 (t, J=7.6 Hz, 1H), 7.32-7.42 (m, 2H), 7.20-7.26 (m, 1H), 6.82 (d, J=6.8 Hz, 1H), 6.58 (d, J=8.0 Hz, 1H), 5.10-5.20 (m, 1H), 4.75-4.89 (m, 2H), 4.66-4.75 (m, 1H), 4.48-4.55 (m, 1H), 3.98-4.46 (m, 1H), 4.08 (s, 3H), 3.93-4.03 (m, 3H), 3.84 (d, J=14.0 Hz, 1H), 2.67-2.84 (m, 3H), 2.40-2.53 (m, 1H), 2.25-2.36 (m, 2H), 1.80-1.92 (m, 1H), 1.48-1.63 (m, 2H). ¹⁹F-NMR (377 MHz): −114.57.

### Example 73: 2-{[4-({6-[(4-chloro-2-fluorophenyl)methyl]pyridin-2-yl}oxy)piperidin-1-yl]methyl}-1-{[(2S)-oxetan-2-yl]methyl}-1H-1,3-benzodiazole-5-carboxylic acid (Compound 188a)

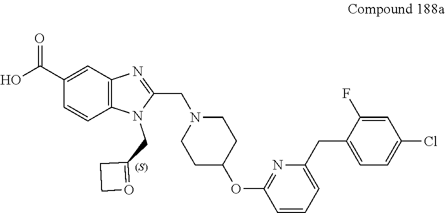

2-{[4-({6-[(4-chloro-2-fluorophenyl)methyl]pyridin-2-yl}oxy)piperidin-1-yl]methyl}-1-{[(2S)-oxetan-2-yl]methyl}-1H-1,3-benzodiazole-5-carboxylic acid Compound 188a (76 mg) was obtained with the similar procedure of Compound 134a as a white solid.

MS Calcd.: 564.2; MS Found: 565.4 [M+H]⁺.

¹H NMR (400 MHz, DMSO-d₆): δ 8.17 (s, 1H), 7.84 (dd, J=8.4 Hz, 1.2 Hz, 1H), 7.70 (d, J=8.4 Hz, 1H), 7.60 (t, J=8.0 Hz, 1H), 7.35-7.40 (m, 2H), 7.23 (dd, J=8.4 Hz, 2.0 Hz, 1H), 6.82 (d, J=7.2 Hz, 1H), 6.58 (d, J=8.0 Hz, 1H), 5.06-5.13 (m, 1H), 4.71-4.86 (m, 2H), 4.57-4.64 (m, 1H), 4.46-4.53 (m, 1H), 4.34-4.42 (m, 1H), 4.01 (s, 2H), 3.92 (d, J=13.6 Hz, 1H), 3.78 (d, J=13.6 Hz, 1H), 2.62-2.82 (m, 3H), 2.37-2.48 (m, 1H), 2.21-2.31 (m, 2H), 1.81-1.90 (m, 2H), 1.48-1.61 (m, 2H). ¹⁹F-NMR (377 MHz): −114.58.

### Example 74: 2-[(4-chloro-2-fluorophenyl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine (Compound 189a)

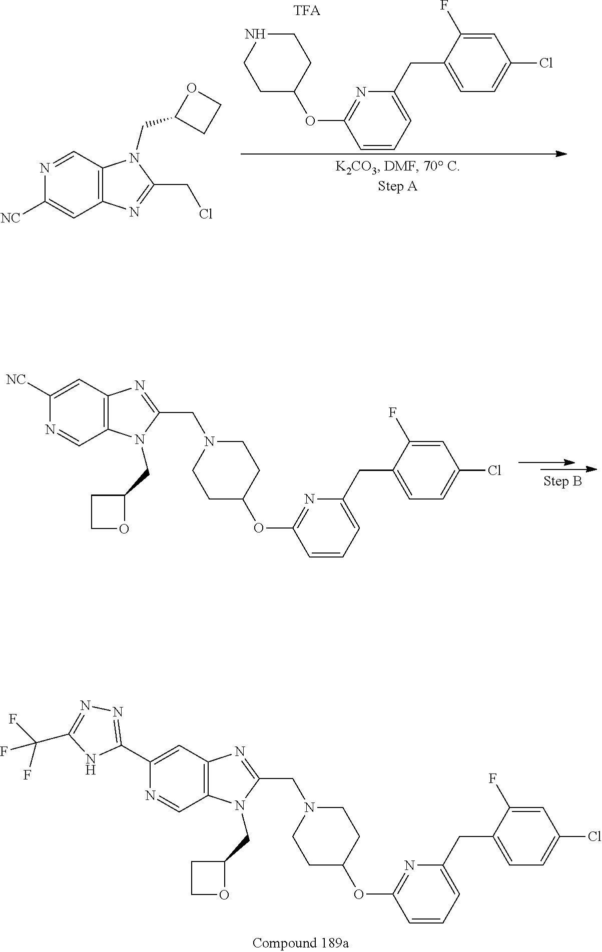

### Step A: (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbonitrile

A mixture of 2-(4-chloro-2-fluorobenzyl)-6-(piperidin-4-yloxy)pyridine TFA salt (159 mg, 0.5 mmol), (S)-2-(chloromethyl)-3-(oxetan-2-ylmethyl)-3H-imidazo[4,5-c]pyridine-6-carbonitrile (100 mg, 0.4 mmol), K₂CO₃ (157 mg, 1.1 mmol) in DMF (4 mL) was stirred at 70° C. for 1 hour. After the reaction was completed, the mixture was diluted with EA (30 mL), washed with H₂O (20 mL*2). The combined organic layer was dried over anhydrous sodium sulfate, filtered, and evaporated to dryness. The residue was purified by column chromatography on silica gel (DCM/MeOH=50/1) to give (S)-2-((4-((6-(4-chloro-2-fluorobenzyl)pyridin-2-yl)oxy)piperidin-1-yl)methyl)-3-(oxetan-2-ylmethyl)-3H-imidazo[4,5-c]pyridine-6-carbonitrile (100 mg, yield: 48%) as yellow oil. MS Calcd.: 546.2; MS Found: 547.2 [M+H]⁺.

### Step B: 2-[(4-chloro-2-fluorophenyl)methyl]-6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridine

(S)-2-((4-((6-(4-chloro-2-fluorobenzyl)pyridin-2-yl)oxy)piperidin-1-yl)methyl)-3-(oxetan-2-ylmethyl)-6-(5-(trifluoromethyl)-4H-1,2,4-triazol-3-yl)-3H-imidazo[4,5-c]pyridine Compound 189a (70 mg) was obtained with the similar procedure of Compound 149a. MS Calcd.: 656.2; MS Found: 657.2 [M+H]⁺.

### Example 75: 3-fluoro-4-{[6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridin-2-yl]methyl}benzonitrile (Compound 190a)

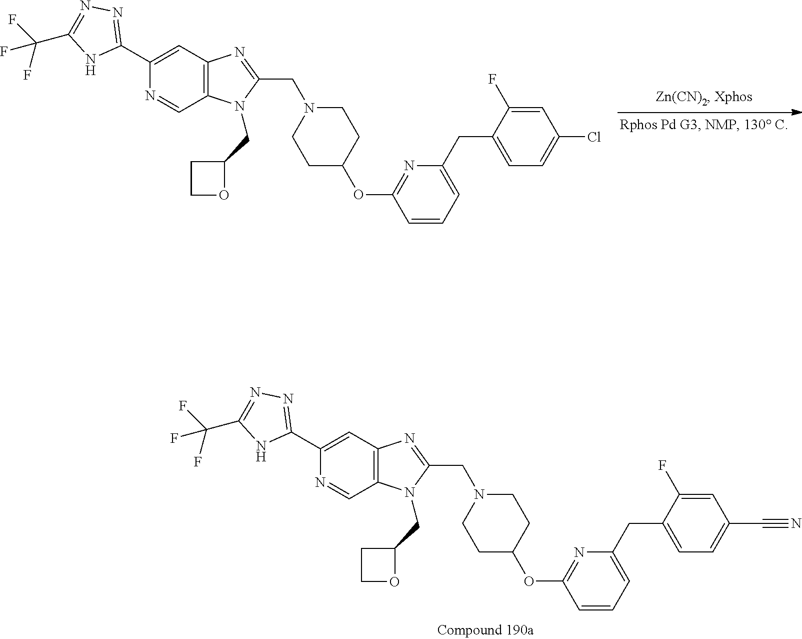 [SMILES: Fc1cc(Cl)ccc1Cc1cccc(OC2CCN(Cc3nc4cc(-c5nnc(C(F)(F)F)[nH]5)ncc4n3C[C@@H]3CCO3)CC2)n1 ; N#Cc1ccc(Cc2cccc(OC3CCN(Cc4nc5cc(-c6nnc(C(F)(F)F)[nH]6)ncc5n4C[C@@H]4CCO4)CC3)n2)c(F)c1]

A mixture of (S)-2-((4-((6-(4-chloro-2-fluorobenzyl)pyridin-2-yl)oxy)piperidin-1-yl)methyl)-3-(oxetan-2-ylmethyl)-6-(5-(trifluoromethyl)-4H-1,2,4-triazol-3-yl)-3H-imidazo[4,5-c]pyridine (50 mg, 0.08 mmol), Zn(CN)₂ (26.8 mg, 0.23 mmol), RuPhos Pd G₃ (12.7 mg, 0.015 mmol) and X-Phos (10.9 mg, 0.023 mmol) in anhydrous NMP (1.0 mL) was stirred at 130° C. for 2 h under Ar. The reaction mixture was filtered and the filtrate purified by Prep-HPLC (0.1% FA/H₂O/CH₃CN) to give 3-fluoro-4-{[6-({1-[(3-{[(2S)-oxetan-2-yl]methyl}-6-[5-(trifluoromethyl)-4H-1,2,4-triazol-3-yl]-3H-imidazo[4,5-c]pyridin-2-yl)methyl]piperidin-4-yl}oxy)pyridin-2-yl]methyl}benzonitrile Compound 190a (8 mg) as a white solid.

MS Calcd.: 647.2; MS Found: 648.1 [M+H]⁺.

¹H NMR (400 MHz, DMSO-d6): δ 9.15 (s, 1H), 8.28 (s, 1H), 7.81 (d, J=9.2 Hz, 1H), 7.53-7.68 (m, 3H), 6.87 (d, J=7.2 Hz, 1H), 6.59 (d, J=8.4 Hz, 1H), 5.10-5.20 (m, 1H), 4.87-4.94 (m, 1H), 4.70-4.83 (m, 2H), 4.48-4.55 (m, 1H), 4.38-4.45 (m, 1H), 4.11 (s, 2H), 3.99 (d, J=14.0 Hz, 1H), 3.85 (d, J=13.2 Hz, 1H), 2.65-2.83 (m, 3H), 2.40-2.55 (m, 1H), 2.23-2.35 (m, 2H), 1.78-1.87 (m, 2H), 1.47-1.60 (m, 2H). ¹⁹F-NMR (377 MHz): −63.61, −114.70.

### Example 76: 2-[(4-chloro-2-fluorophenyl)methyl]-6-[(1-{[3-(oxan-4-yl)-6-[5-(trifluoromethyl)-4H-1,2,4-triazol-3-yl]-3H-imidazo[4,5-c]pyridin-2-yl]methyl}piperidin-4-yl)oxy]pyridine (Compound 191)

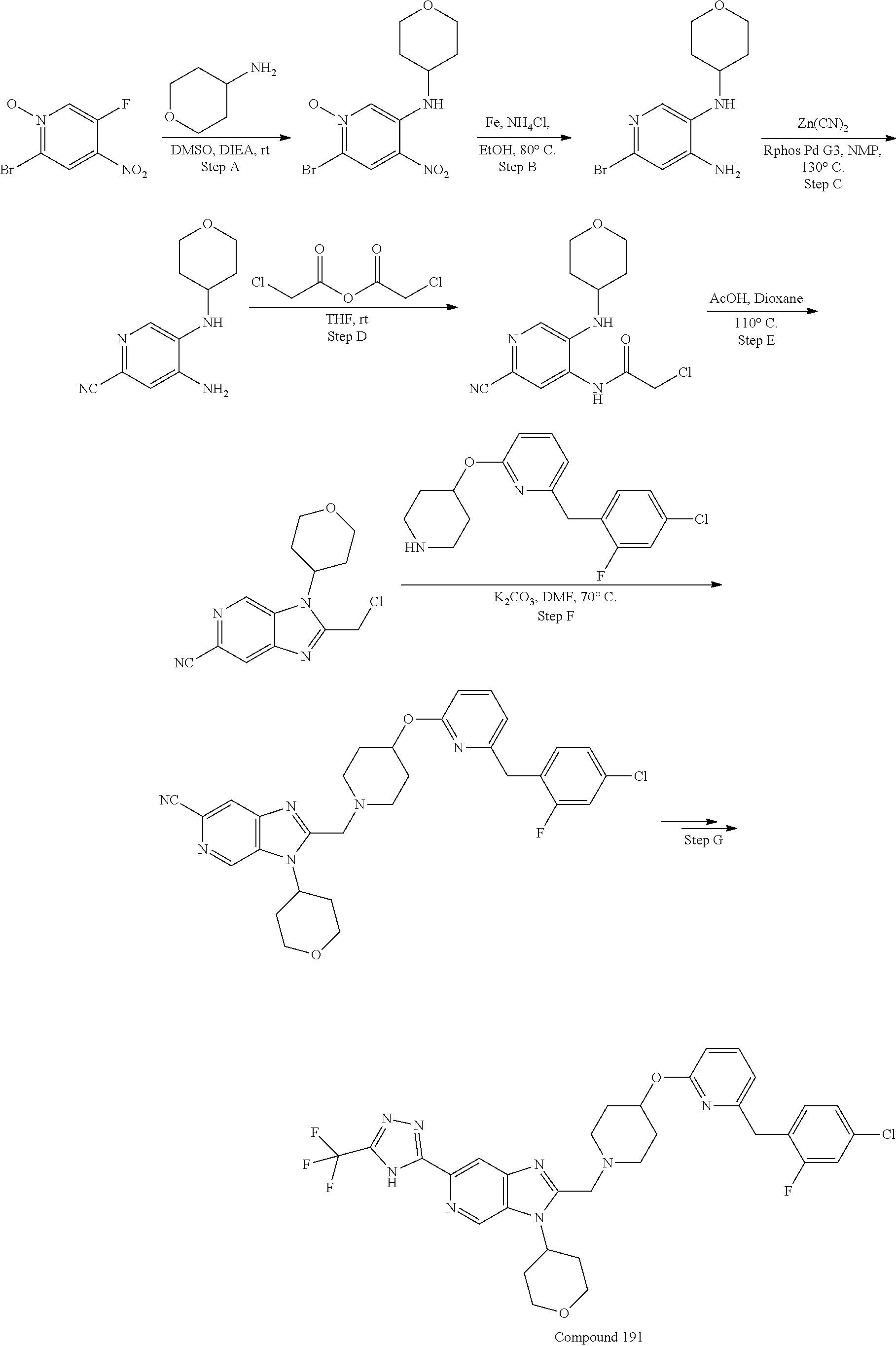

### Step A: 2-bromo-4-nitro-5-((tetrahydro-2H-pyran-4-yl)amino)pyridine 1-oxide

To a mixture of 2-bromo-5-fluoro-4-nitropyridine 1-oxide (2.0 g, 8.5 mmol) and tetrahydro-2H-pyran-4-amine (778 mg, 7.7 mmol) in DMSO (20.0 mL) was added DIEA (3.0 g, 23.1 mmol). The mixture was stirred at rt for 2 hours. After the reaction was completed, the mixture was diluted with EA (100 mL), washed with H₂O (50 mL*2). The organic layer was evaporated to give 2-bromo-4-nitro-5-((tetrahydro-2H-pyran-4-yl)amino)pyridine 1-oxide (2.0 g, yield: 82%) as yellow solid. MS Calcd.: 317.0; MS Found: 318.0 [M+H]⁺.

### Step B: 6-bromo-N3-(tetrahydro-2H-pyran-4-yl)pyridine-3,4-diamine

To a mixture of 2-bromo-4-nitro-5-((tetrahydro-2H-pyran-4-yl)amino)pyridine 1-oxide (2.0 g, 6.3 mmol) in EtOH (20.0 mL) and H₂O (4.0 mL) was added NH₄Cl (1.7 g, 31.5 mmol) and Fe (1.8 g, 31.5 mmol). The mixture was stirred at 80° C. for 3 hours. After the reaction was completed, the mixture was filtered, and the filtrate was quenched with H₂O (15.0 mL) and extracted with EA (40 mL*2). The combined organic layer was dried over anhydrous sodium sulfate, filtered, and concentrated. The residue was purified by column chromatography on silica gel (0.1% NH₃·H₂O/PE/EA=1:1) to give crude 6-bromo-N3-(tetrahydro-2H-pyran-4-yl)pyridine-3,4-diamine (0.7 g, yield: 41%) as a purple solid. MS Calcd.: 271.0; MS Found: 272.1 [M]⁺.

### Step C: 4-amino-5-((tetrahydro-2H-pyran-4-yl)amino)picolinonitrile

A mixture of 6-bromo-N3-(tetrahydro-2H-pyran-4-yl)pyridine-3,4-diamine (700 mg, 2.6 mmol) and Zn(CN)₂ (903 mg, 7.7 mmol), RuPhos Pd G₃ (215 mg, 0.26 mmol) and X-Phos (122 mg, 0.26 mmol) in anhydrous NMP (7.0 mL) was stirred at 130° C. for 30 mins under N₂. After the reaction was completed, the mixture was quenched with H₂O (10.0 mL) and extracted with EA (20 mL*2). The combined organic layer was dried over anhydrous sodium sulfate, filtered and evaporated to dryness. The residue was purified by column chromatography on silica gel (DCM/MeOH=10:1) to give 4-amino-5-((tetrahydro-2H-pyran-4-yl)amino)picolinonitrile (NMP residual, 600 mg, crude) as yellow oil. MS Calcd.: 218.1; MS Found: 219.1 [M+H]⁺.

### Step D: 2-chloro-N-(2-cyano-5-((tetrahydro-2H-pyran-4-yl)amino)pyridin-4-yl)acetamide

A mixture of 4-amino-5-((tetrahydro-2H-pyran-4-yl)amino)picolinonitrile (600 mg, 2.7 mmol) and 2-chloroacetic anhydride (611 mg, 3.6 mmol) in THE (6 mL) was stirred at rt for 16 hours. After the reaction was completed, the mixture was quenched with H₂O (10.0 mL) and extracted with EA (20 mL*2). The combined organic layer was dried over anhydrous sodium sulfate, filtered to give 2-chloro-N-(2-cyano-5-((tetrahydro-2H-pyran-4-yl)amino)pyridin-4-yl)acetamide (636 mg, yield: 80%) as yellow oil. MS Calcd: 294.1; MS Found: 295.1 [M+H]⁺.

### Step E: 2-(chloromethyl)-3-(tetrahydro-2H-pyran-4-yl)-2,3-dihydro-1H-imidazo[4,5-c]pyridine-6-carbonitrile

To a solution of (2-cyano-5-((tetrahydro-2H-pyran-4-yl)amino)pyridin-4-yl)glycinoyl chloride (626 mg, 2.1 mmol) in AcOH (10 mL) and dioxane (2 mL) was stirred at 110° C. for 3 hours. The clear reaction solution was then quenched with saturated aqueous sodium bicarbonate solution, diluted with EtOAc (20 mL), washed with water (15 mL). The organic layers were combined, dried over Na₂SO₄, filtered and concentrated under vacuum to give 2-(chloromethyl)-3-(tetrahydro-2H-pyran-4-yl)-2,3-dihydro-1H-imidazo[4,5-c]pyridine-6-carbonitrile (367 mg, 62% yield) as a white solid. MS Calcd.: 276.1; MS Found: 277.1 [M+H]⁺.

### Step F: 2-((4-((6-(4-chloro-2-fluorobenzyl)pyridin-2-yl)oxy)piperidin-1-yl)methyl)-3-(tetrahydro-2H-pyran-4-yl)-3H-imidazo[4,5-c]pyridine-6-carbonitrile

To a mixture of 2-(chloromethyl)-3-(tetrahydro-2H-pyran-4-yl)-2,3-dihydro-1H-imidazo[4,5-c]pyridine-6-carbonitrile (200 mg, 0.7 mmol) in DMF (5 mL) was added 2-(4-chloro-2-fluorobenzyl)-6-(piperidin-4-yloxy)pyridine (690 mg, 2.2 mmol). The resulting mixture was stirred at 70° C. for 1 hour under an atmosphere of nitrogen (balloon). The reaction mixture was diluted with EtOAc (20 mL), washed with water (15 mL). The organic layers were combined, dried over Na₂SO₄, filtered and concentrated under vacuum. The residue was purified by silica gel chromatography, eluting with PE:EA=1:1 to give 2-((4-((6-(4-chloro-2-fluorobenzyl)pyridin-2-yl)oxy)piperidin-1-yl)methyl)-3-(tetrahydro-2H-pyran-4-yl)-3H-imidazo[4,5-c]pyridine-6-carbonitrile (295 mg, 73% yield) as a brown solid. MS Calcd.: 560.2; MS Found: 561.0 [M+H]⁺.

### Step G: 2-[(4-chloro-2-fluorophenyl)methyl]-6-[(1-{[3-(oxan-4-yl)-6-[5-(trifluoromethyl)-4H-1,2,4-triazol-3-yl]-3H-imidazo[4,5-c]pyridin-2-yl]methyl}piperidin-4-yl)oxy]pyridine

2-[(4-chloro-2-fluorophenyl)methyl]-6-[(1-{[3-(oxan-4-yl)-6-[5-(trifluoromethyl)-4H-1,2,4-triazol-3-yl]-3H-imidazo[4,5-c]pyridin-2-yl]methyl}piperidin-4-yl)oxy]pyridine (Compound 191) (5 mg) was obtained with the similar procedure of Compound 149a as a white solid.

MS Calcd.: 670.22; MS Found: 671.1 [M+H]⁺.

¹H NMR (400 MHz, DMSO-d₆): δ 9.18 (s, 1H), 8.31 (s, 1H), 7.59 (d, J=8.0 Hz, 1H), 7.35-7.40 (m, 2H), 7.23 (d, J=7.6 Hz, 1H), 6.82 (d, J=7.2 Hz, 1H), 6.57 (d, J=8.4 Hz, 1H), 4.78-5.01 (m, 2H), 4.08-4.16 (m, 2H), 3.95-4.03 (m, 4H), 3.50-3.60 (m, 2H), 2.70-2.78 (m, 2H), 2.40-2.53 (m, 2H), 2.23-2.34 (m, 2H), 1.81-1.98 (m, 4H), 1.43-1.55 (m, 2H). ¹⁹F-NMR (377 MHz): −63.71, −114.59.

### Example 77: 2-[(4-chloro-2-fluorophenyl)methyl]-6-{[1-({3-[(1-methoxycyclopropyl)methyl]-6-[5-(trifluoromethyl)-4H-1,2,4-triazol-3-yl]-3H-imidazo[4,5-c]pyridin-2-yl}methyl)piperidin-4-yl]oxy}pyridine (Compound 192)

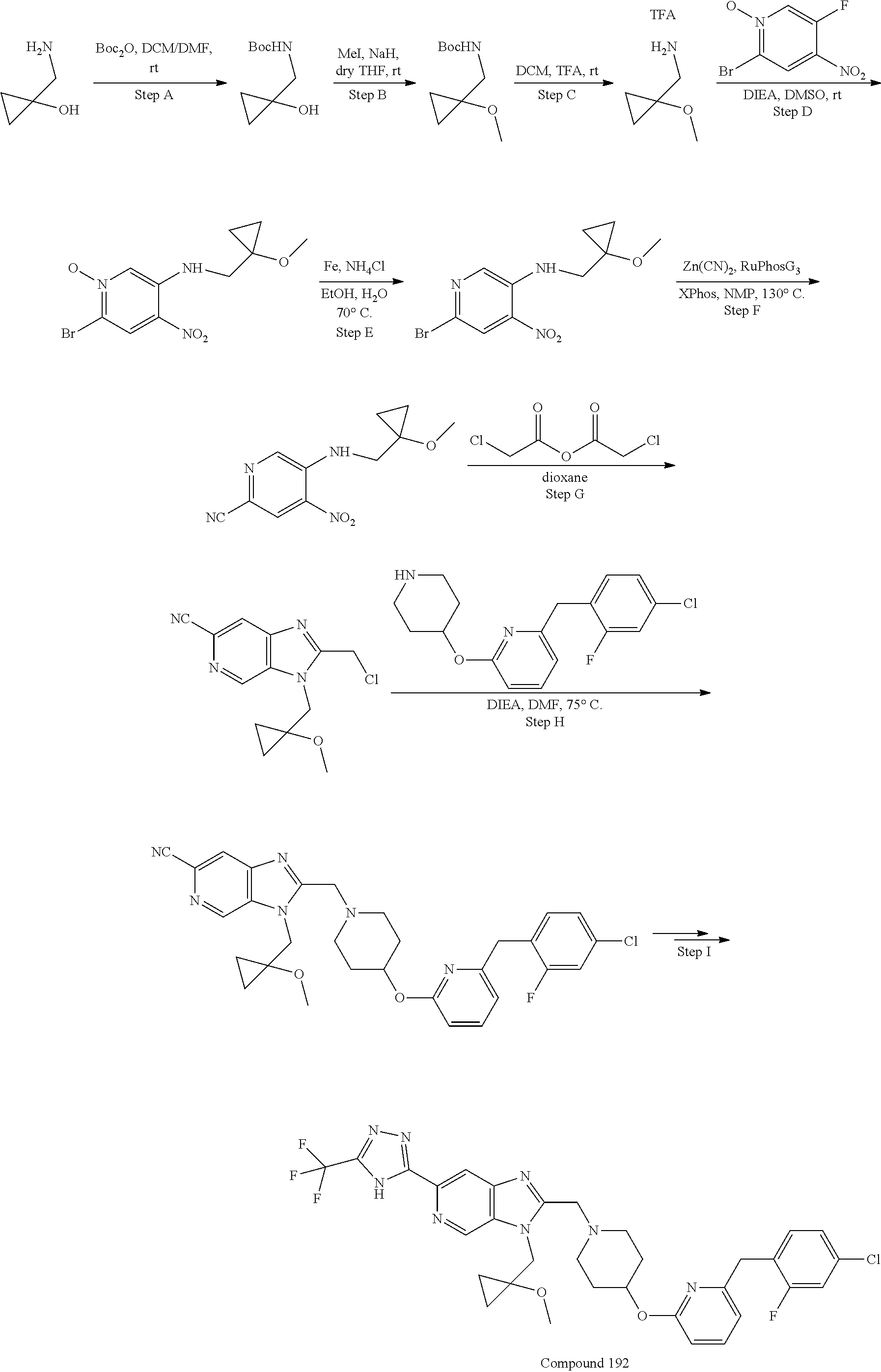

### Step A: tert-butyl ((1-hydroxycyclopropyl)methyl)carbamate

To a solution of 1-(aminomethyl)cyclopropan-1-ol (1.0 g, 11.5 mmol) in DCM/DMF (8 mL/2 mL) was added Boc₂O (2.5 g, 11.5 mmol). The mixture was stirred at rt for 16 hours. After the reaction was completed, the mixture was concentrated, diluted with EA (20 mL), washed with brine (20 mL×3). The organic layer was dried over anhydrous sodium sulfate, filtered, and evaporated to dryness. The residue was purified by column chromatography on silica gel (PE/EA=3/1) to give tert-butyl ((1-hydroxycyclopropyl)methyl)carbamate (1.6 g, 74% yield) as a white solid.

¹H NMR (400 MHz, DMSO-d₆): δ 6.66 (t, J=4.8 Hz, 1H), 5.20 (s, 1H), 3.08 (d, J=5.6 Hz, 2H), 1.37 (s, 9H), 0.40-0.45 (m, 4H).

### Step B: tert-butyl ((1-methoxycyclopropyl)methyl)carbamate

To a solution of tert-butyl ((1-hydroxycyclopropyl)methyl)carbamate (1.6 g, 8.7 mmol) in dry THF (10 mL) was added NaH (411 mg, 60% purity, 10.3 mmol) at 0° C. 0.5 h later, MeI (1.46 g, 10.27 mmol) was added. The mixture was stirred at rt for 1 hour. After the reaction was completed, the mixture was diluted with EA (20 mL), washed with brine (20 mL×3). The organic layer was dried over anhydrous sodium sulfate, filtered, and evaporated to dryness. The residue was purified by column chromatography on silica gel (PE/EA=4/1) to give tert-butyl ((1-methoxycyclopropyl)methyl)carbamate (1.2 g, 70% yield) as a yellow oil.

¹H NMR (400 MHz, DMSO-d₆): δ 6.85 (t, J=6.0 Hz, 1H), 3.18 (s, 3H), 3.15 (d, J=6.4 Hz, 2H), 1.38 (s, 9H), 0.43-0.62 (m, 4H).

### Step C: (1-methoxycyclopropyl)methanamine

To a solution of tert-butyl ((1-methoxycyclopropyl)methyl)carbamate (1.2 g, 6.0 mmol) in DCM (3 mL) was added TFA (1 mL). The mixture was stirred at room temperature for 1 hour. After the reaction was completed, the mixture was concentrated to give (1-methoxycyclopropyl)methanamine (1.6 g, crude, TFA salt) as a brown oil.

### Step D: 2-bromo-5-(((1-methoxycyclopropyl)methyl)amino)-4-nitropyridine 1-oxide

To a mixture of (1-methoxycyclopropyl)methanamine TFA salt (1.6 g, 7.4 mmol) in DMSO (10 mL) was added DIEA (2.6 g, 20.3 mmol). 5 min later, 2-bromo-5-fluoro-4-nitropyridine 1-oxide (1.6 g, 6.8 mmol) was added. The mixture was stirred at room temperature for 2 hours. After the reaction was completed, the mixture was diluted with EA (50 mL), washed with brine (50 mL×3). The organic layer was dried over anhydrous sodium sulfate, filtered, and evaporated to dryness. The residue was purified by column chromatography on silica gel (MeOH/DCM=1/20) to give 2-bromo-5-(((1-methoxycyclopropyl)methyl)amino)-4-nitropyridine-1-oxide (276 mg, 12% yield) as a yellow solid. MS Calcd.: 317.0; MS Found: 318.0 [M+H]⁺.

### Step E: 6-bromo-N3-((1-methoxycyclopropyl)methyl)pyridine-3,4-diamine

To a mixture of 2-bromo-5-(((1-methoxycyclopropyl)methyl)amino)-4-nitropyridine 1-oxide (256 mg, 0.80 mmol) in EtOH (10 mL) and H₂O (0.5 mL) was added Fe (230 mg, 4.0 mmol) and NH₄Cl (230 mg, 4.0 mmol). The mixture was stirred at 70° C. for 2 hours. After the reaction was completed, the mixture was filtrated. The filtrate was purified by column chromatography on silica gel (MeOH/DCM=1/15) to give 6-bromo-N3-((1-methoxycyclopropyl)methyl)pyridine-3,4-diamine (130 mg, 60% yield) as a purple solid. MS Calcd.: 271.0; MS Found: 272.0 [M+H]⁺.

### Step F: 4-amino-5-(((1-methoxycyclopropyl)methyl)amino)picolinonitrile

A mixture of 6-bromo-N3-((1-methoxycyclopropyl)methyl)pyridine-3,4-diamine (110 mg, 0.40 mmol), Zn(CN)₂ (95 mg, 0.81 mmol), RuPhos Pd G₃ (68 mg, 0.081 mmol) and XPhos (39 mg, 0.081 mmol) in NMP (2 mL) was stirred at 130° C. for 20 min. After the reaction was completed, the mixture was diluted with EA (10 mL×3), washed with brine (10 mL×5). The organic layer was dried over anhydrous sodium sulfate, filtered, and evaporated in vacuo to dryness. The residue was concentrated and purified by column chromatography (MeOH/DCM=1/10) to give 4-amino-5-(((1-methoxycyclopropyl)methyl)amino)picolinonitrile (100 mg, impure) as a yellow solid. MS Calcd.: 218.1; MS Found: 219.1 [M+H]⁺.

### Step G: 2-(chloromethyl)-3-((1-methoxycyclopropyl)methyl)-3H-imidazo[4,5-c]pyridine-6-carbonitrile

A mixture of 4-amino-5-(((1-methoxycyclopropyl)methyl)amino) picolinonitrile (100 mg, impure) and 2-chloroacetic anhydride (156 mg, 0.92 mmol) in dioxane (10 mL) was stirred at rt for 48 hours. Then, the mixture was heated to 70° C. for 48 hours. After the reaction was completed, the mixture was diluted with EA (10 ml×3), washed with saturated NaHSO₃ (5 mL). The organic layer was dried over anhydrous sodium sulfate, filtered, and evaporated in vacuo to dryness. The residue was purified by column chromatography (MeOH/DCM=1/20) to give 2-(chloromethyl)-3-((1-methoxycyclopropyl)methyl)-3H-imidazo[4,5-c]pyridine-6-carbonitrile (40 mg, 32% yield) as a yellow solid. MS Calcd.: 276.1; MS Found: 277.1 [M+H]⁺.

### Step H: 2-((4-((6-(4-chloro-2-fluorobenzyl)pyridin-2-yl)oxy)piperidin-1-yl)methyl)-3-((1-methoxycyclopropyl)methyl)-3H-imidazo[4,5-c]pyridine-6-carbonitrile

A mixture of 2-(chloromethyl)-3-((1-methoxycyclopropyl)methyl)-3H-imidazo[4,5-c]pyridine-6-carbonitrile (40 mg, 0.145 mmol), 2-(4-chloro-2-fluorobenzyl)-6-(piperidin-4-yloxy)pyridine (144 mg, 0.435 mmol) and DIEA (57 mg, 0.435 mmol) in DMF (2 mL) was stirred at 75° C. for 1 hour. After the reaction was completed, the mixture was diluted with EA (20 ml×3), washed with brine (5 ml×5). The organic layer was dried over anhydrous sodium sulfate and evaporated in vacuo to dryness. The residue was purified by column chromatography (MeOH/DCM=1/10) to give 2-((4-((6-(4-chloro-2-fluorobenzyl)pyridin-2-yl)oxy)piperidin-1-yl)methyl)-3-((1-methoxycyclopropyl)methyl)-3H-imidazo[4,5-c]pyridine-6-carbonitrile (60 mg, 75% yield) as a yellow oil. MS Calcd.: 560.2; MS Found: 561.3 [M+H]⁺.

### Step I: 2-[(4-chloro-2-fluorophenyl)methyl]-6-{[1-({3-[(1-methoxycyclopropyl)methyl]-6-[5-(trifluoromethyl)-4H-1,2,4-triazol-3-yl]-3H-imidazo[4,5-c]pyridin-2-yl}methyl)piperidin-4-yl]oxy}pyridine

2-[(4-chloro-2-fluorophenyl)methyl]-6-{[1-({3-[(1-methoxycyclopropyl)methyl]-6-[5-(trifluoromethyl)-4H-1,2,4-triazol-3-yl]-3H-imidazo[4,5-c]pyridin-2-yl}methyl)piperidin-4-yl]oxy}pyridine Compound 192 (2 mg) was obtained with the similar procedure of Compound 149a as a white solid.

MS Calcd.: 670.2; MS Found: 671.1 [M+H]⁺.

¹H NMR (400 MHz, DMSO-d₆): δ 9.14 (s, 1H), 8.28 (s, 1H), 7.60 (t, J=7.6 Hz, 1H), 7.32-7.41 (m, 2H), 7.23 (dd, J=1.6 Hz, J=8.0 Hz, 1H), 6.82 (d, J=7.2 Hz, 1H), 6.58 (d, J=8.4 Hz, 1H), 4.76-4.87 (m, 3H), 4.00 (s, 2H), 3.92 (s, 2H), 3.26 (s, 3H), 2.70-2.81 (m, 2H), 2.22-2.35 (m, 2H), 1.82-1.92 (m, 2H), 1.49-1.61 (m, 2H), 0.85-0.94 (m, 4H). ¹⁹F-NMR (377 MHz): −63.47, −114.58.

### Example 78: 5-(2-{[4-({2-[(4-chloro-2-fluorophenyl)methyl]-5-fluoropyrimidin-4-yl}oxy)piperidin-1-yl]methyl}-3-{[(2S)-oxetan-2-yl]methyl}-3H-imidazo[4,5-c]pyridin-6-yl)-4H-1,2,4-triazole-3-carbonitrile (Compound 193a)

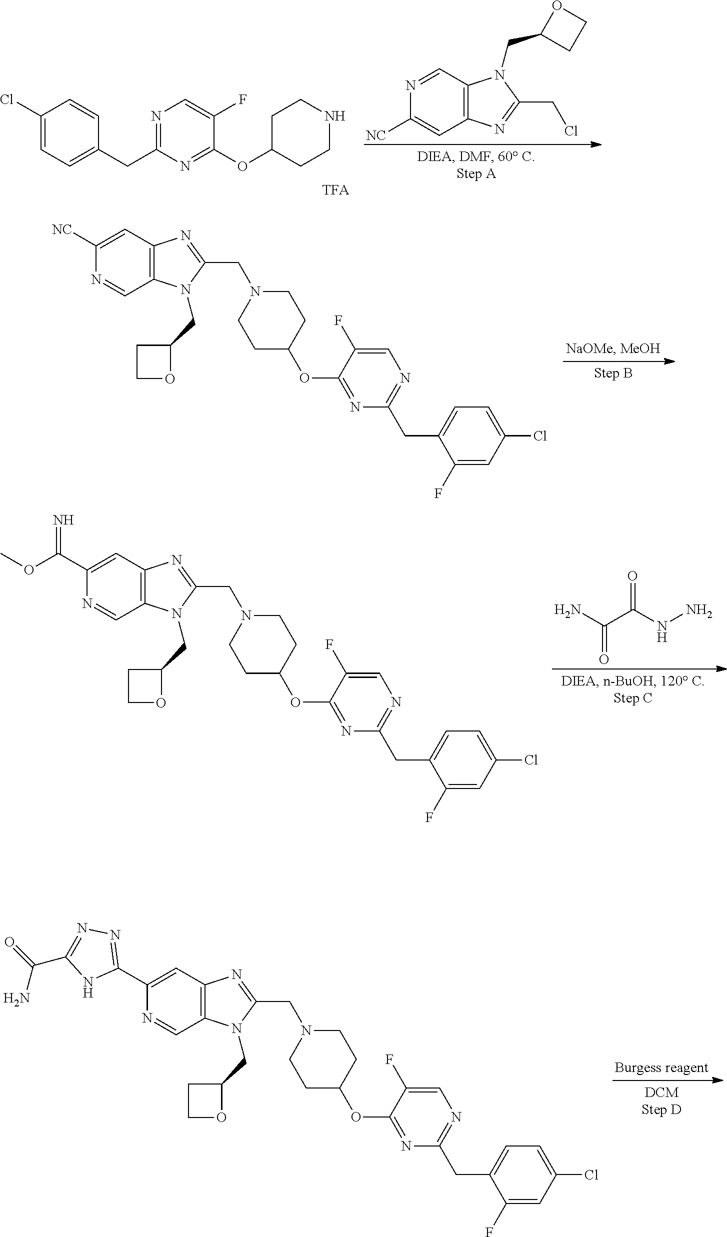

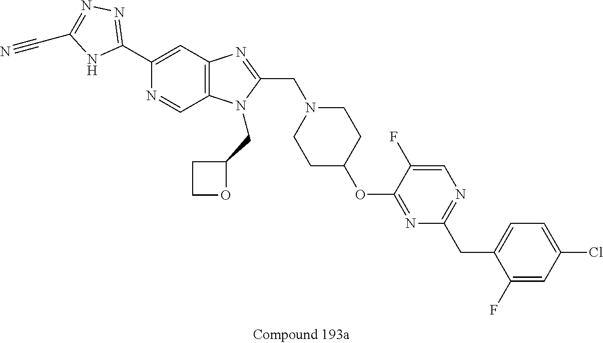 [SMILES: N#Cc1nnc(-c2cc3nc(CN4CCC(Oc5nc(Cc6ccc(Cl)cc6F)ncc5F)CC4)n(C[C@@H]4CCO4)c3cn2)[nH]1]

### Step A: The synthesis of (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbonitrile

A mixture of 2-(4-chloro-2-fluorobenzyl)-5-fluoro-4-(piperidin-4-yloxy)pyrimidine TFA salt (750 mg, crude, 1.14 mmol), (S)-2-(chloromethyl)-3-(oxetan-2-ylmethyl)-3H-imidazo[4,5-c]pyridine-6-carbonitrile (150 mg, 0.57 mmol), DIEA (738.55 mg, 5.73 mmol) in DMF (3.0 mL) was stirred at 60° C. for 2 hours. After the reaction was completed, the mixture was diluted with EA (40 mL), washed with H2O (30 mL×2). The combined organic layer was dried over anhydrous sodium sulfate, filtered and evaporated to dryness. The residue was purified by column chromatography on silica gel (DCM/MeOH=80/1) to give (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbonitrile (270 mg, yield: 84%) as a brown solid. MS Calcd.: 565.2; MS Found: 566.1 [M+H]⁺.

### Step B: The synthesis of methyl (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbimidate

A mixture of (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbonitrile (270 mg, 0.48 mmol), Sodium methoxide (258 mg, 4.78 mmol) in MeOH (4.0 mL) was stirred at room temperature for 1 hour. After the reaction was completed, the mixture was diluted with EA (40 mL), washed with H2O (30 mL×2). The combined organic layer was dried over anhydrous sodium sulfate, filtered and evaporated to dryness. The mixture was concentrated in vacuum to give methyl (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbimidate (250 mg, yield: 88%) as a brown solid. MS Calcd.: 597.2; MS Found: 598.2 [M+H]⁺.

### Step C: The synthesis of (S)-5-(2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridin-6-yl)-4H-1,2,4-triazole-3-carboxamide

To a solution of methyl (S)-2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridine-6-carbimidate (250 mg, 0.42 mmol) in n-BuOH (4 mL) was added DIEA (170 mg, 1.26 mmol) and 2-hydrazineyl-2-oxoacetamide (86 mg, 0.84 mmol) at room temperature. The reaction was stirred at 120° C. for 16 hours. After the reaction was completed, the reaction was concentrated, quenched with water (5 mL) and extracted with ethyl acetate (10 mL×3). The organic layer was combined and washed with brine (5 mL), dried over sodium sulfate, filtered and concentrated in vacuum. The residue was purified by column chromatography on silica gel (MeOH/DCM=20/1) to give (S)-5-(2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridin-6-yl)-4H-1,2,4-triazole-3-carboxamide (67 mg, yield: 25%) as a white solid. MS Calcd.: 650.2; MS Found: 651.2 [M+H]⁺.

### Step D: The synthesis of 5-(2-{[4-({2-[(4-chloro-2-fluorophenyl)methyl]-5-fluoropyrimidin-4-yl}oxy)piperidin-1-yl]methyl}-3-{[(2S)-oxetan-2-yl]methyl}-3H-imidazo[4,5-c]pyridin-6-yl)-4H-1,2,4-triazole-3-carbonitrile (Compound 193a)

To a mixture of (S)-5-(2-((4-((2-(4-chloro-2-fluorobenzyl)-5-fluoropyrimidin-4-yl)oxy)piperidin-1-yl)methyl)-3-(oxetan-2-ylmethyl)-3H-imidazo[4,5-c]pyridin-6-yl)-4H-1,2,4-triazole-3-carboxamide (67 mg, 0.10 mmol), Burgess reagent (98 mg, 0.41 mmol) in DCM (2.0 mL) was stirred at rt for 1 hour. After the reaction was completed, the mixture was diluted with EA (20 mL), washed with H2O (10 mL×2). The combined organic layer was dried over anhydrous sodium sulfate, filtered, and evaporated to dryness. The organic layer was concentrated and purified by Prep-HPLC (0.1% FA/H2O/CH3CN) to give 5-(2-{[4-({2-[(4-chloro-2-fluorophenyl)methyl]-5-fluoropyrimidin-4-yl}oxy)piperidin-1-yl]methyl}-3-{[(2S)-oxetan-2-yl]methyl}-3H-imidazo[4,5-c]pyridin-6-yl)-4H-1,2,4-triazole-3-carbonitrile Compound 193a (23 mg, yield: 36%) as a white solid. MS Calcd.: 632.2; MS Found: 633.1 [M+H]+.

¹H NMR (400 MHz, DMSO-d₆): δ 9.17 (s, 1H), 8.50 (d, J=3.2 Hz, 1H), 8.31 (s, 1H), 7.33-7.44 (m, 2H), 7.25 (dd, J=8.4 Hz, 1.6 Hz, 1H), 5.11-5.19 (m, 1H), 4.86-5.03 (m, 2H), 4.71-4.78 (m, 1H), 4.48-4.56 (m, 1H), 4.37-4.45 (m, 1H), 4.15 (s, 2H), 4.01 (d, J=14.0 Hz, 1H), 3.87 (d, J=13.6 Hz, 1H), 2.68-2.85 (m, 3H), 2.42-2.47 (m, 1H), 2.29-2.41 (m, 2H), 1.87-1.96 (m, 2H), 1.61-1.73 (m, 2H). ¹⁹F NMR (400 MHz, DMSO-d₆): δ −114.14, −157.34.

### Example A: cAMP Assays

Activation of GLP-1 receptor is known to stimulate cyclic AMP (cAMP) production in cells which indicates primary coupling to the G_{αs} subunit of the G protein heterotrimeric complex. Evidence suggests signaling through G_{αs} induced cAMP stimulation elicits the desired pharmacological response regarding insulin release from pancreatic β-cells.

To optimize functional activity directed toward G_{αs} coupling, a HEK293/CRE-Luc cell line developed by HDB stably expressing the GLP-1 Receptor was used. 200× concentration of compound working solutions were prepared (Agilent Technologies Bravo) with ½ log serial dilution in 384-well Echo LDV plate (Labcyte, Cat #LP-0200). 50 nL/well 200× concentration of compound working solutions were moved to 384-well white low volume plate (Greiner, Cat #784075) using Labcyte ECHO550. 1×10⁵ cells/mL HEK293/GLP1R/CRE-LUC(HD Biosciences) cell suspensions prepared with assay buffer[DPBS containing 0.5 mM IBMX(Sigma, Cat #15879) and 0.1% BSA (GENVIEW, Cat #FA016-100 g)], 10 uL cell suspensions were added to each well of previous generated assay plate which already contains 50 nl compound at 200×concentration using ThermoFisher Multidrop Combi (1000 cells/well). Seal the plate and incubate at 37° C. with 5% CO₂ for 30 min.

After incubation the cAMP assay signal was generated using cAMP dynamic 2 Kit (Cisbio). 5 μL cAMP-d2 working solution was added to each well, followed with 5 μL Anti-cAMP antibody-cryptate working solution added to each well using ThermoFisher Multidrop Combi. Incubate at room temperature for 1 hour protected from light. Read the fluorescence at 665 and 615 nm with Reader PerkinElmer EnVision.

% Activity=100%×(mean RLU of test sample−mean RLU of vehicle control)/(mean RLU of MAX control−mean RLU of vehicle control))

Table 1 shows the biological activity of compounds in GLP-1R agonist cAMP stimulation assay (EC₅₀)

| Compound No. | EC₅₀ (nM) |
|---|---|
| 101a | 0.081 |
| 102a | 0.31 |
| 103a | 0.093 |
| 104a | 1.1 |
| 105a | 5.8 |
| 106a | 0.098 |
| 107a | 0.2 |
| 108a | 0.38 |
| 109a | 15 |
| 110a | 0.38 |
| 111a | 0.089 |
| 112a | 0.37 |
| 113a | 0.18 |
| 114a | 1.6 |
| 115a | 2.7 |
| 116a | 11 |
| 117a | 5.9 |
| 118a | 44 |
| 119a | 43 |
| 120a | 0.17 |
| 121a | 0.5 |
| 122a | 0.54 |
| 123 | 5.7 |
| 124a | 0.59 |
| 126a | 0.32 |
| 129a | 7.6 |
| 130a | 0.79 |
| 131a | 1.8 |
| 134a | 0.053 |
| 135a | 0.19 |
| 136a | 0.22 |
| 137a | 0.14 |
| 138a | 0.03 |
| 140a | 1.7 |
| 141a | 0.13 |
| 142a | 0.048 |
| 143a | 0.22 |
| 144a | 1.8 |
| 145a | 0.082 |
| 147a | 11 |
| 148a | 1.4 |
| 149a | 0.056 |
| 150a | 0.036 |
| 153 | 0.18 |
| 155 | 0.32 |
| 157 | 1.1 |
| 160a | 12.2 |
| 162a | 2.9 |
| 163a | 0.25 |
| 164a | 0.35 |
| 165a | 12 |
| 166a | 1.8 |
| 167a | 0.23 |
| 168a | 0.081 |
| 169a | 1.5 |
| 170a | 0.13 |
| 171a | 0.17 |
| 172a | 1.8 |
| 173a | 0.81 |
| 174a | 3.4 |
| 175a | 0.17 |
| 176a | 0.2 |
| 177a | 0.13 |
| 178 | 0.2 |
| 179 | 0.49 |
| 180a | 0.65 |
| 181a | 0.58 |
| 182a | 0.2 |
| 183a | 3.3 |
| 184a | 13 |
| 185a | 0.37 |
| 186a | 4.4 |
| 187a | >300 |
| 188a | 16 |
| 189a | / |
| 190a | 0.24 |
| 191 | 41 |
| 192 | 4 |
| 193a | 0.024 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A compound of Formula I: 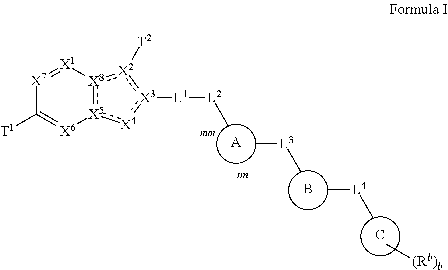
or a pharmaceutically acceptable salt or solvate thereof, wherein: [symbol]
indicates an optional single or double bond, as allowed by valence;
each of X¹, X², X³, X⁴, X⁵, X⁶, X⁷, and X⁸ is independently selected from the group consisting of C, CH, CR^{w}, and N, provided that at least two and no more than four of X¹, X², X³, X⁴, X⁵, X⁶, X⁷, and X⁸ are N;
each R^{w} is independently selected from the group consisting of: halogen; cyano; (C₁-C₆)alkyl; (C₁-C₆)haloalkyl; (C₁-C₆)alkoxy; and (C₁-C₃)haloalkoxy;
T¹ is selected from the group consisting of: -T³ and -L^{a}-(CR^{x}R^{x})_{q}-T³;
T³ is selected from the group consisting of:
—N(R^{s})C(═O)R^{z};
—N(R^{s})C(═O)OR^{z};
—N(R^{s})C(═O)N(R^{s})R^{z};
—N(R^{s})S(O)₁₋₂—R^{z};
—N(R^{s})S(═NR^{s})(═O)R^{z};
—S(O)₁₋₂R^{z};
—P(═O)R^{z1}R^{z2};
—C(═O)OH;
—C(═O)N(R^{s})R^{z};
—S(O)₁₋₂N(R^{s})R^{z};
—S(═NR^{s})(═O)N(R^{s})R^{z};
5- to 10-membered heteroaryl optionally substituted with 1-4 R^{v}, and wherein the heteroaryl optionally comprises an endocyclic group selected from the group consisting of: 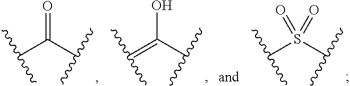
5- to 10-membered heterocycloalkyl, wherein the heterocycloalkyl comprises an endocyclic group selected from the group consisting of: 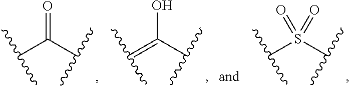
wherein the heterocycloalkyl is optionally substituted with 1-4 R^{v}; and
(C₁-C₆)haloalkyl substituted with —OH and further optionally substituted with 1-2 R^{v};
L^{a} is a bond, —NH—, —N(C₁₋₃ alkyl)-, O, or S(O)₀₋₂;
q is 0, 1, 2, or 3, provided that when q is 0, then L^{a} is other than a bond;
each R^{s} is independently selected from the group consisting of: hydrogen, (C₁-C₆)alkyl, and (C₃-C₈)cycloalkyl;
each R^{x} is independently selected from the group consisting of: hydrogen, halogen, (C₁-C₆)alkyl, and (C₁-C₃)haloalkyl; or
a pair of R^{x} taken together with the carbon atom to which each is attached forms a (C₃-C₈)cycloalkyl ring;
or when q is 2 or 3, a pair of R^{x} on adjacent carbon atoms, taken together, form a double bond between the adjacent carbon atoms;
R^{z}, R^{z1}, and R^{z2} are each independently selected from the group consisting of: hydrogen; (C₁-C₆)alkyl optionally substituted with 1-4 R^{v}; —R^{z3}; and -L^{b}-R^{z3}; or
R^{z1} and R^{z2} taken together with the phosphorous atom to which each is attached forms a ring including from 5-8 ring atoms, wherein from 0-2 ring atoms (in addition to the phosphorous attached to R^{z1} and R^{z2}) are heteroatoms each independently selected from the group consisting of: O, S, and N, wherein the ring is optionally substituted with 1-3 independently selected (C₁-C₆)alkyl;
L^{b} is C₁₋₃ alkylene optionally substituted with 1-4 R^{v};
R^{z3} is selected from the group consisting of: (C₃-C₆)cycloalkyl, 3- to 6-membered heterocycloalkyl, (C₆-C₁₀)aryl, and 5- to 10-membered heteroaryl, each of which is optionally substituted with 1-4 R^{v};
each occurrence of R^{v} is independently selected from the group consisting of: (C₁-C₆)alkyl, (C₁-C₆)haloalkyl, (C₁-C₆)alkoxy, (C₁-C₆)haloalkoxy, CN, —O(C₁-C₆)alkylene-O(C₁-C₆)alkyl, phenyl, —S(O)₀₋₂—C₁-C₆ alkyl, and halogen;
T² is hydrogen or (C₁-C₆)alkyl which is optionally substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T};
each R^{T} is independently selected from the group consisting of: OH, SH, CN, NO₂, halogen, (C₁-C₆)alkyl, (C₂-C₆)alkenyl, (C₂-C₆)alkynyl, (C₁-C₆)haloalkyl, (C₁-C₆)cyanoalkyl, (C₁-C₆)hydroxyalkyl, (C₁-C₆)alkoxy, (C₁-C₆)haloalkoxy, (C₃-C₆)cycloalkyl, amino, (C₁-C₆)alkylamino, and di(C₁-C₆)alkylamino;
L¹ is a bond or (C₁-C₃)alkylene which is optionally substituted with 1-3 R^{L};
L² is a bond, —O—, —S(O)₀₋₂—, or —NH—;
each R^{L} is independently selected from the group consisting of: halogen, (C₁-C₃)alkyl, and (C₁-C₃)haloalkyl; or a pair of R^{L} on the same or on adjacent carbon atoms, taken together with the atom(s) to which each is attached, forms a (C₃-C₆)cycloalkyl ring;
Ring A is selected from the group consisting of: 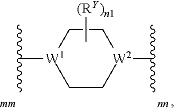
wherein n1 is 0, 1, or 2; W¹ is CR^{Y1} or N; and W² is CR^{Y2} or N; 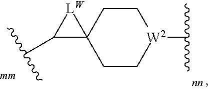
where W² is CR^{Y2} or N, L^{w} is (C₁-C₃)alkylene;
phenylene optionally substituted with 1-4 R^{Y};
5- to 6-membered heteroarylene optionally substituted with 1-3 R^{Y};
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y}; 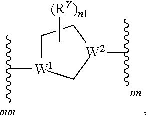
wherein n1 is 0, 1, or 2; W¹ is CR^{Y1} or N; and W² is CR^{Y2} or N; wherein mm represents the point of attachment to L², and nn represents the point of attachment to L³;
each occurrence of R^{Y} is independently selected from the group consisting of halogen, CN, —OH, oxo, (C₁-C₆)alkyl, (C₁-C₃)haloalkyl, (C₁-C₃)alkoxy, and (C₁-C₃)haloalkoxy;
R^{Y1} and R^{Y2} are each independently selected from the group consisting of hydrogen, halogen, CN, —OH, (C₁-C₆)alkyl, (C₁-C₃)haloalkyl, (C₁-C₃)alkoxy, and (C₁-C₃)haloalkoxy; or
when W¹ is CR^{Y1} and W² is CR^{Y2}, the R^{Y1} and R^{Y2} groups taken together form (C₁-C₄)alkylene, wherein one of the CH₂ units of the (C₁-C₄)alkylene is optionally replaced by a heteroatom selected from the group consisting of O, S, NH, and N(C₁₋₃)alkyl;
L³ is selected from the group consisting of: —O—; —S—; —C(R^{a}R^{a})—; —N(H)—; —N(C₁₋₃ alkyl)-; —C(═O)—; and —S(O)₁₋₂—,
each occurrence of R^{a} is independently selected from the group consisting of: hydrogen, halogen, CN, —OH, (C₁-C₆)alkyl, (C₁-C₃)haloalkyl, (C₃-C₈)cycloalkyl, (C₁-C₃)alkoxy, and (C₁-C₃)haloalkoxy; or
a pair of R^{a} taken together with the carbon atom to which each is attached forms a (C₃-C₈)cycloalkyl ring; or
when Ring A is 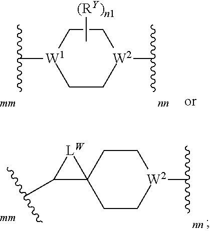
W² is CR^{Y2}; and L³ is —C(R^{a}R^{a})—, one R^{a} combines with R^{Y2} to form a double bond between W² and L³, wherein the remaining R^{a} is selected from the group consisting of: hydrogen, halogen, CN, (C₁-C₆)alkyl, (C₁-C₃)haloalkyl, and (C₃-C₈)cycloalkyl;
Ring B is selected from the group consisting of: (B-I), (B-II), (B-III), (B-IV), and (B-V): 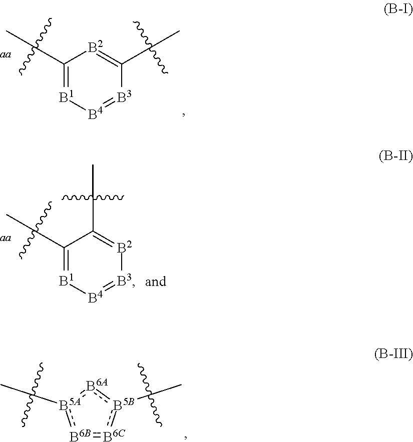
wherein aa represents the point of attachment to L³;
each of B¹, B², B³, and B⁴ is independently selected from the group consisting of CR¹ and N;
each of B^{5A} and B^{5B} is independently selected from the group consisting of: C and N,
each of B^{6A}, B^{6B}, and B^{6C} is independently selected from the group consisting of: O, S, CR¹, NR^{N}, and N,
each [symbol] in (B-III) is independently a single bond or a double bond,
provided that at least one of B^{5A}, B^{5B}, B^{6A}, B^{6B}, and B^{6C} is an independently selected heteroatom, at least one of B^{5A}, B^{5B}, B^{6A}, B^{6B}, and B^{6C} is C or CR¹, and the ring including B^{5A}, B^{5B}, B^{6A}, B^{6B}, and B^{6C} is heteroaryl; 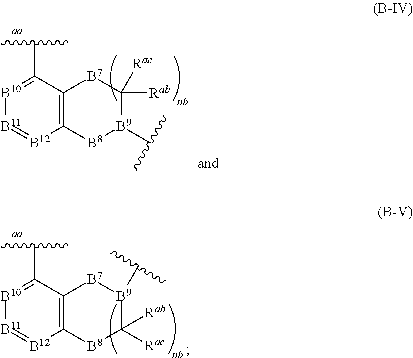
wherein aa represents the point of attachment to L³;
B⁷ and B⁸ are independently selected from the group consisting of: —O—, —NR^{N}—, and —C(R¹)₂—;
B⁹ is N or CR^{aa};
nb is 0 or 1;
B¹⁰, B¹¹, and B¹² are independently selected from the group consisting of CR¹ and N;
each R¹ is selected from the group consisting of: hydrogen, halogen, CN, (C₁-C₆)alkyl, (C₁-C₆)haloalkyl; (C₁-C₃)alkyl(C₃-C₆)cycloalkyl, (C₁-C₃)alkyl(3- to 5-membered heterocycloalkyl), and —C(O)NR²R³;
each R² and R³ is independently selected from the group consisting of: H and (C₁-C₆)alkyl;
each R^{N} is selected from the group consisting of: hydrogen, (C₁-C₆)alkyl, (C₁-C₆)haloalkyl, C(═O)(C₁-C₆)alkyl, S(O)₂(C₁-C₆)alkyl, and C(═O)O(C₁-C₆)alkyl;
R^{aa}, R^{ab}, and R^{ac} are each independently selected from the group consisting of H, (C₁-C₆)alkyl, and (C₁-C₆)haloalkyl;
L⁴ is a bond or —Z¹—Z²—*, wherein * represents the point of attachment to Ring C;
Z¹ and Z² are independently selected from the group consisting of: a bond, NH, N(C₁-C₆ alkyl), O, C(═O), S(O)₀₋₂, and C₁₋₃ alkylene optionally substituted with 1-2 R^{c};
provided that Z¹ and Z² are not simultaneously a bond;
further provided that when Z¹ is NH, N(C₁-C₆ alkyl), —O—, or —S—, then Z² is a bond, C(═O), S(O)₁₋₂, or C₁₋₃ alkylene optionally substituted with 1-2 R^{c}; and
when Z² is NH, N(C₁-C₆ alkyl), —O—, or —S—, then Z¹ is a bond, C(═O), S(O)₁₋₂, or C₁₋₃ alkylene optionally substituted with 1-3 R^{c};
each R^{c} is independently selected from the group consisting of halogen, (C₁-C₆)alkyl, and (C₁-C₃)haloalkyl, or a pair of R^{c} taken together with the carbon atom to which each is attached forms a (C₃-C₈)cycloalkyl ring;
Ring C is selected from the group consisting of phenyl, 5- to 6-membered heteroaryl, (C₃-C₆)cycloalkyl, (C₅-C₁₀)bicycloalkyl, 5- to 10-membered bicycloheteroaryl, and 3- to 6-membered heterocycloalkyl;
each R^{b} is independently selected from the group consisting of (C₁-C₆)alkyl, (C₁-C₆)haloalkyl, (C₁-C₆)alkoxy, (C₁-C₆)haloalkoxy, halogen, (C₃-C₆)cycloalkyl, and CN; and
b is an integer selected from 0-3.

2. The compound of claim 1, wherein T¹ is -T³.

3. The compound of claim 1, wherein T¹ is -L^{a}-(CR^{x}R^{x})_{q}-T³.

4. The compound of claims 1 or 3, wherein -L^{a} is a bond.

5. The compound of any one of claims 1 or 3-4, wherein -L^{a} is —NH—, —N(C₁₋₃ alkyl)-, —O—, or —S—.

6. The compound of any one of claims 1 or 3-5, wherein -L^{a} is —O—.

7. The compound of any one of claims 1 or 3-6, wherein q is 1.

8. The compound of any one of claims 1 or 3-6, wherein q is 2 or 3.

9. The compound of any one of claims 1 or 3-8, wherein each R^{x} is hydrogen or (C₁-C₆)alkyl.

10. The compound of any one of claims 1 or 3-9, wherein each R^{x} is hydrogen.

11. The compound of any one of claims 1 or 3-8, wherein one pair of R^{x} on the same carbon, taken together with the carbon atom to which each is attached, forms a (C₃-C₈)cycloalkyl ring.

12. The compound of any one of claims 1, 3-8, or 11, wherein one pair of R^{x} on the same carbon, taken together with the carbon atom to which each is attached, forms a cyclopropyl.

13. The compound of claims 11 or 12, wherein each remaining R^{x} is hydrogen.

14. The compound of claims 1 or 3, wherein T¹ is —(CR^{x}R^{x})-T³.

15. The compound of claim 14, wherein each R^{x} is hydrogen.

16. The compound of claim 14, wherein the pair of R^{x} taken together with the carbon atom to which each is attached, forms a (C₃-C₈)cycloalkyl ring.

17. The compound of claims 1 or 3, wherein T¹ is —(CR^{x}R^{x})_{q}-T³; and q is 2 or 3.

18. The compound of claim 17, wherein each R^{x} is hydrogen.

19. The compound of claims 1 or 3, wherein T¹ is —O—(CR^{x}R^{x})_{q}-T³; and q is 1, 2, or 3.

20. The compound of claim 19, wherein q is 1.

21. The compound of claims 19 or 20, wherein each R^{x} is hydrogen.

22. The compound of any one of claims 1-21, wherein T³ is selected from the group consisting of: —N(R^{s})C(═O)R^{z}, —N(R^{s})C(═O)OR^{z}, —N(R^{s})C(═O)N(R^{s})R^{z}, —N(R^{s})S(O)₁₋₂—R^{z}, and —N(R^{s})S(═NR^{s})(═O)R^{z}.

23. The compound of any one of claims 1-22, wherein T3 is —N(R^{s})C(═O)R^{z}.

24. The compound of any one of claims 1-23, wherein T³ is —NHC(═O)R^{z}.

25. The compound of any one of claims 1-22, wherein T³ is —N(R^{s})S(O)₁₋₂—R^{z}.

26. The compound of any one of claims 1-22 or 25, wherein T³ is —NHS(O)₂R^{z}.

27. The compound of any one of claims 1-21, wherein T³ is —S(O)₁₋₂R^{z}.

28. The compound of any one of claims 1-21 or 27, wherein T³ is —S(O)₂R^{z}.

29. The compound of any one of claims 1-28, wherein R^{z} is (C₁-C₆)alkyl optionally substituted with 1-4 R^{v}.

30. The compound of any one of claims 1-29, wherein R^{z} is (C₁-C₃)alkyl optionally substituted with 1-4 R^{v}.

31. The compound of any one of claims 1-30, wherein R^{z} is (C₁-C₃)alkyl.

32. The compound of any one of claims 1-31, wherein R^{z} is methyl, ethyl, or isopropyl.

33. The compound of any one of claims 1-30, wherein R^{z} is (C₁-C₃)alkyl substituted with 1-3 R^{v}.

34. The compound of any one of claims 1-30 or 33, wherein R^{z} is (C₁-C₃)alkyl substituted with from 1-3 substituents each independently selected from the group consisting of halo and (C₁-C₃)alkoxy, such as wherein R^{z} is —CH₂CF₃ or —CH₂CH₂OMe.

35. The compound of any one of claims 1-28, wherein R^{z} is —R^{z3} or -L^{b}-R^{z3}.

36. The compound of any one of claims 1-28 or 35, wherein R^{z} is —R^{z3} or —CH₂—R^{z3}.

37. The compound of any one of claims 1-28 or 35-36, wherein R^{z3} is selected from the group consisting of: (C₃-C₆)cycloalkyl and 3- to 6-membered heterocycloalkyl, each of which is optionally substituted with 1-4 R^{v}.

38. The compound of any one of claims 1-28 or 35-37, wherein R^{z3} is selected from the group consisting of: (C₃-C₆)cycloalkyl and 3- to 6-membered heterocycloalkyl, each optionally substituted with 1-2 R^{v}.

39. The compound of any one of claims 1-28 or 35-38, wherein R^{z3} is cyclopropyl, cyclobutyl, difluorocyclobutyl, cycopentyl, oxetanyl, tetrahydropyranyl, or tetrahydro-2H-thiopyran 1,1-dioxide.

40. The compound of any one of claims 1-28 or 35-36, wherein R^{z3} is selected from the group consisting of: (C₆-C₁₀)aryl and 5- to 10-membered heteroaryl, each of which is optionally substituted with 1-4 R^{v}.

41. The compound of any one of claims 1-28, 35-36, or 40, wherein R^{z3} is phenyl optionally substituted with 1-2 R^{v}.

42. The compound of any one of claims 1-21, wherein T³ is —NHC(═O)R^{z} or —NHS(O)₁₋₂—R^{z}; and R^{z} is (C₁-C₆)alkyl optionally substituted with 1-4 R^{v}.

43. The compound of claim 40, wherein R^{z} is (C₁-C₃)alkyl optionally substituted with (C₁-C₃)alkoxy; or wherein R^{z} is (C₁-C₃)alkyl substituted with 1-3 halo.

44. The compound of any one of claims 1-21, wherein T³ is —NHC(═O)R^{z} or —NHS(O)₁₋₂—R^{z}; and R^{z} is —R^{z3} or —CH₂—R^{z3}.

45. The compound of claim 42, wherein R^{z3} is selected from the group consisting of: phenyl, (C₃-C₆)cycloalkyl, and 3- to 6-membered heterocycloalkyl, each optionally substituted with 1-2 R^{v}, such as wherein R^{z3} is phenyl, cyclopropyl, cyclobutyl, difluorocyclobutyl, cycopentyl, oxetanyl, tetrahydropyranyl, or tetrahydro-2H-thiopyran 1,1-dioxide.

46. The compound of any one of claims 1-21, wherein T³ is —S(O)₂R^{z}; and R^{z} is (C₁-C₆)alkyl optionally substituted with 1-4 R^{v}.

47. The compound of any one of claims 1-21, wherein T³ is —C(═O)OH.

48. The compound of any one of claims 1-21, wherein T³ is —P(═O)R^{z1}R^{z2}.

49. The compound of any one of claims 1-21 or 48, wherein R^{z1} and R^{z2} are each independently selected (C₁-C₃)alkyl.

50. The compound of any one of claims 1-21, wherein T³ is 5- to 10-membered heteroaryl optionally substituted with 1-4 R^{v}, and wherein the heteroaryl optionally comprises an endocyclic group selected from the group consisting of:

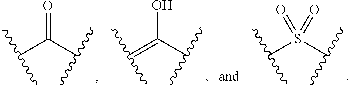

51. The compound of any one of claims 1-21 or 50, wherein T³ is 5- to 6-membered heteroaryl optionally substituted with 1-4 R^{v}.

52. The compound of any one of claims 1-21 or 50-51, wherein T³ is 5-membered heteroaryl having 2-4 ring heteroatoms selected from the group consisting of N, O, and S, wherein the heteroaryl is optionally substituted with 1-2 R^{v}.

53. The compound of any one of claims 1-21 or 50-52, wherein T³ is selected from the group consisting of:

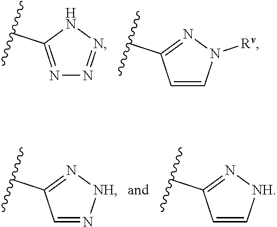

54. The compound of any one of claims 1-21 or 50, wherein T³ is 5- to 6-membered heteroaryl that comprises an endocyclic group selected from the group consisting of: 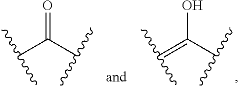
wherein the heteroaryl is further optionally substituted with 1-4 R^{v}.

55. The compound of any one of claims 1-21, 50, or 54, wherein T³ is selected from the group consisting of:

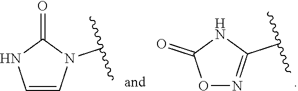 [SMILES: *n1cc[nH]c1=O ; *c1noc(=O)[nH]1]

56. The compound of any one of claims 1-21, wherein T³ is 5- to 10-membered heterocycloalkyl, wherein the heterocycloalkyl comprises an endocyclic group selected from the group consisting of: 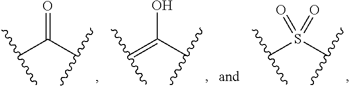
wherein the heterocycloalkyl is optionally substituted with 1-4 R^{v}.

57. The compound of any one of claims 1-21 or 56, wherein T³ is 5- to 6-membered heterocycloalkyl which comprises an endocyclic group selected from the group consisting of: 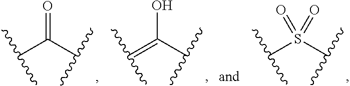
wherein the heterocycloalkyl is optionally substituted with 1-4 R^{v}.

58. The compound of any one of claims 1-21 or 56-57, wherein T³ is 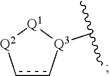
Q¹ is C(═O) or S(O)₂; Q² is O, NH, —CH₂—, or —CH₂—CH₂—; Q³ is N or CH; and [symbol] is a single bond or a double bond, provided that T³ is non-aromatic.

59. The compound of any one of claims 1-21 or 56-58, wherein T³ is selected from the group consisting of:

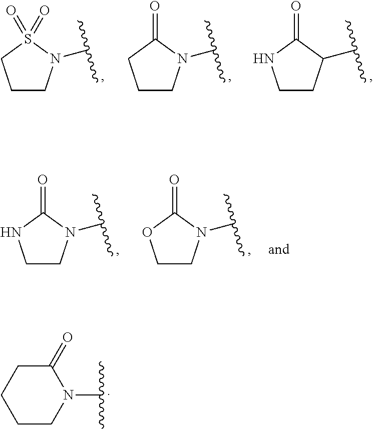

60. The compound of claim 1, wherein T is —N(R^{s})C(═O)R^{z} or —N(R^{s})S(O)₁₋₂—R^{z}; and R^{z} is (C₁-C₆)alkyl optionally substituted with 1-4 R^{v}.

61. The compound of claim 60, wherein R^{z} is (C₁-C₃)alkyl.

62. The compound of claim 60, wherein R^{z} is (C₁-C₃)alkyl substituted with from 1-3 substituents each independently selected from the group consisting of halo and (C₁-C₃)alkoxy, such as wherein R^{z} is —CH₂CF₃ or —CH₂CH₂OMe.

63. The compound of claim 1, wherein T¹ is —N(R^{s})C(═O)R^{z} or —N(R^{s})S(O)₁₋₂—R^{z}; and R^{z} is —R^{z3} or —CH₂—R^{z3}.

64. The compound of claim 63, wherein R^{z3} is selected from the group consisting of: phenyl, (C₃-C₆)cycloalkyl, and 3- to 6-membered heterocycloalkyl, each optionally substituted with 1-2 R^{v}, such as wherein R^{z3} is phenyl, cyclopropyl, cyclobutyl, difluorocyclobutyl, cycopentyl, oxetanyl, tetrahydropyranyl, or tetrahydro-2H-thiopyran 1,1-dioxide.

65. The compound of any one of claims 60-64, wherein T¹ is —NHC(═O)R^{z}.

66. The compound of any one of claims 60-64, wherein T¹ is —NHS(O)₂—R^{z}.

67. The compound of claim 1, wherein T¹ is —(CR^{x}R^{x})_{q}—S(O)₂R^{z}; and q is 1, 2, or 3.

68. The compound of claim 67, wherein R^{z} is (C₁-C₆)alkyl optionally substituted with 1-4 R^{v}.

69. The compound of claims 67 or 68, wherein R^{z} is (C₁-C₃)alkyl.

70. The compound of any one of claims 67-69, wherein q is 1.

71. The compound of any one of claims 67-70, wherein each R^{x} is hydrogen.

72. The compound of claim 1, wherein T¹ is —(CR^{x}R^{x})_{q}—C(═O)OH or —C(═O)OH; and q is 1, 2, or 3.

73. The compound of claim 72, wherein q is 1.

74. The compound of claim 72, wherein q is 2.

75. The compound of any one of claims 72-74, wherein each R^{x} is hydrogen.

76. The compound of any one of claims 72-74, wherein one pair of R^{x} on the same carbon, taken together with the carbon atom to which each is attached, forms a (C₃-C₈)cycloalkyl ring; and each remaining R^{x} when present is hydrogen.

77. The compound of claim 76, wherein the pair of R^{x} on the same carbon, taken together with the carbon atom to which each is attached, forms a cyclopropyl ring.

78. The compound of claims 1 or 72, wherein T¹ is C(═O)OH,

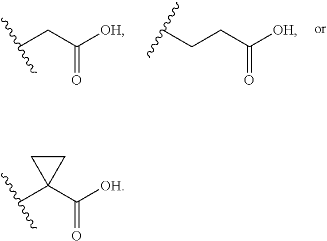 [SMILES: *CC(=O)O ; *CCC(=O)O ; "or" ; *C1(C(=O)O)CC1]

79. The compound of claim 1, wherein T¹ is —O—(CR^{x}R^{x})_{q}—C(═O)OH; and q is 1, 2, or 3.

80. The compound of claim 79, wherein q is 1.

81. The compound of claims 79 or 80, wherein each R^{x} is hydrogen.

82. The compound of claim 1, wherein T¹ is —P(═O)R^{z1}R^{z2}.

83. The compound of claim 82, wherein R^{z1} and R^{z2} are independently selected (C₁-C₃)alkyl.

84. The compound of claim 1, wherein T¹ is 5-membered heteroaryl having 2-4 ring heteroatoms selected from the group consisting of N, O, and S, wherein the heteroaryl is optionally substituted with 1-2 R^{v}.

85. The compound of claims 1 or 84, wherein T¹ is selected from the group consisting of:

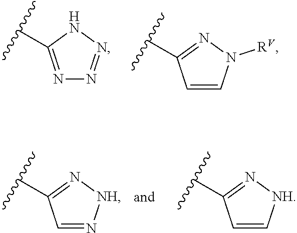

86. The compound of claim 1, wherein T¹ is 5- to 6-membered heteroaryl that comprises an endocyclic group selected from the group consisting of: 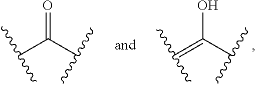
wherein the heteroaryl is further optionally substituted with 1-4 R^{v}.

87. The compound of claims 1 or 86, wherein T¹ is selected from the group consisting of:

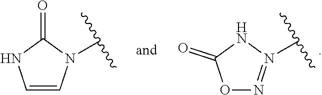

88. The compound of claim 1, wherein T¹ is 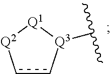
Q¹ is C(═O) or S(O)₂; Q² is O, NH, —CH₂—, or —CH₂—CH₂—; Q³ is N or CH; and [symbol] is a single bond or a double bond, provided that T¹ is non-aromatic.

89. The compound of claims 1 or 88, wherein T¹ is selected from the group consisting of:

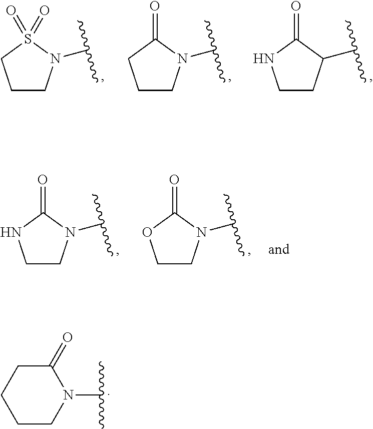

90. The compound of any one of claims 1-89, wherein X² is N; and X⁴ is N.

91. The compound of any one of claims 1-90, wherein X⁸ is C; and X⁵ is C.

92. The compound of any one of claims 1-91, wherein X³ is C.

93. The compound of any one of claims 1-92, wherein X² is N; X³ is C; X⁴ is N; X⁵ is C; and X⁸ is C.

94. The compound of any one of claims 1-93, wherein each of X¹, X⁷, and X⁶ is independently CH or CR^{w}.

95. The compound of any one of claims 1-94, wherein each of X¹, X⁷, and X⁶ is CH.

96. The compound of any one of claims 1-94, wherein one of X¹, X⁷, and X⁶ is CR^{w}; and each remaining of X¹, X⁷, and X⁶ is CH.

97. The compound of any one of claims 1-94 or 96, wherein X⁶ is CR^{w}; and X¹ and X⁷ are CH.

98. The compound of claim 97, wherein X⁶ is C—F.

99. The compound of any one of claims 1-93, wherein one of X¹, X⁷, and X⁶ is N; and each remaining of X¹, X⁷, and X⁶ is CH or CR^{w}.

100. The compound of any one of claims 1-93 or 99, wherein X¹ is N; and X⁶ and X⁷ are CH.

101. The compound of any one of claims 1-93 or 99, wherein X⁶ is N; and X¹ and X⁷ are CH.

102. The compound of any one of claims 1-93 or 99, wherein X⁷ is N; and X¹ and X⁶ are CH.

103. The compound of any one of claims 1-89, wherein the 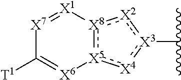
moiety is

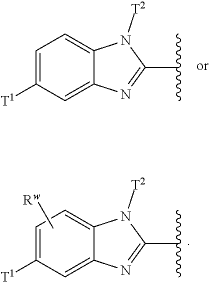

104. The compound of any one of claims 1-89, wherein the 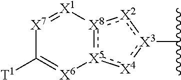
moiety is

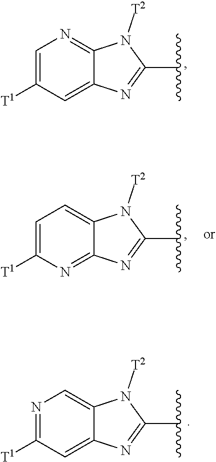

105. The compound of any one of claims 1-104, wherein T² is (C₁-C₆)alkyl which is substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

106. The compound of any one of claims 1-105, wherein T² is (C₁-C₆)alkyl which is substituted with 3- to 6-membered heterocycloalkyl.

107. The compound of any one of claims 1-106, wherein T² is (C₁-C₃)alkyl which is substituted with 4- to 6-membered heterocycloalkyl.

108. The compound of any one of claims 1-107, wherein T² is (C₁-C₃)alkyl which is substituted with oxetanyl.

109. The compound of any one of claims 1-108, wherein T² is

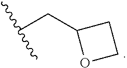 [SMILES: *CC1CCO1]

110. The compound of claim 109, wherein the stereogenic center in 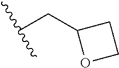 [SMILES: *CC1CCO1]
has (S)-configuration.

111. The compound of any one of claims 1-110, wherein L¹ is (C₁-C₃)alkylene which is optionally substituted with 1-3 R^{L}.

112. The compound of any one of claims 1-111, wherein L¹ is CH₂.

113. The compound of any one of claims 1-110, wherein L¹ is a bond.

114. The compound of any one of claims 1-113, wherein L² is a bond.

115. The compound of any one of claims 1-110, wherein L¹ is CH₂; and L² is a bond.

116. The compound of any one of claims 1-110, wherein L¹ is a bond; and L² is a bond.

117. The compound of any one of claims 1-116, wherein Ring A is

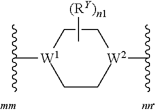

118. The compound of claim 117, wherein W¹ is N.

119. The compound of claims 117 or 118, wherein W² is CR^{Y2}.

120. The compound of claim 119, wherein R^{Y2} is hydrogen.

121. The compound of claims 117 or 118, wherein W² is N.

122. The compound of any one of claims 117-121, wherein n1 is 0.

123. The compound of any one of claims 117-121, wherein n1 is 1.

124. The compound of any one of claims 1-117, wherein Ring A is

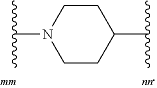 [SMILES: *C1CCN(*)CC1]

125. The compound of any one of claims 1-116, wherein Ring A is

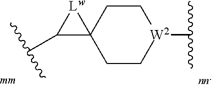

126. The compound of claim 125, wherein L^{w} is CH₂.

127. The compound of claims 125 or 126, wherein W² is N.

128. The compound of any one of claims 1-116 or 125-127, wherein Ring A is

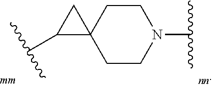 [SMILES: *C1CC12CCN(*)CC2]

129. The compound of any one of claims 1-116, wherein Ring A is selected from the group consisting of:
partially unsaturated monocyclic (C₅-C₈)cycloalkylene optionally substituted with 1-4 R^{Y}; and
partially unsaturated monocyclic 5- to 8-membered heterocycloalkylene optionally substituted with 1-4 R^{Y}.

130. The compound of any one of claims 1-116 or 129, wherein Ring A is 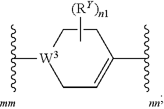
W³ is N or CH; and n1 is 0, 1, or 2.

131. The compound of claim 130, wherein n1 is 0.

132. The compound of claims 130 or 131, wherein W³ is N.

133. The compound of claims 130 or 131, wherein W³ is CH.

134. The compound of any one of claims 1-116 or 130, wherein Ring A is

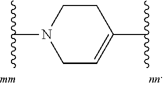 [SMILES: *C1=CCN(*)CC1]

135. The compound of any one of claims 1-116 or 130, wherein Ring A is

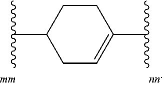 [SMILES: *C1=CCC(*)CC1]

136. The compound of any one of claims 1-110, wherein L² is a bond; L¹ is CH₂; and Ring A is

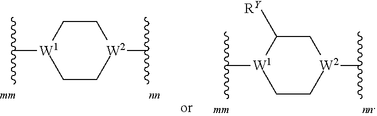

137. The compound of claim 136, wherein Ring A is

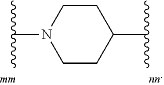 [SMILES: *C1CCN(*)CC1]

138. The compound of any one of claims 1-110, wherein L² is a bond; L¹ is CH₂; and Ring A is

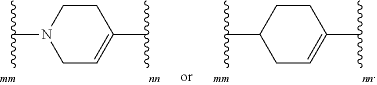 [SMILES: *C1=CCN(*)CC1 ; *C1=CCC(*)CC1]

139. The compound of any one of claims 1-110, wherein L² is a bond; L¹ is a bond; and Ring A is

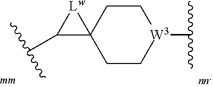

140. The compound of claim 139, wherein Ring A is

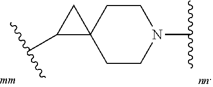 [SMILES: *C1CC12CCN(*)CC2]

141. The compound of any one of claims 1-140, wherein L³ is —O—; —S—; —C(RaRa)—; —N(H)—; —N(C1-3 alkyl)-; —C(═O)—; and —S(O)1-2-.

142. The compound of any one of claims 1-141, wherein L³ is —O—.

143. The compound of any one of claims 1-142, wherein Ring B is

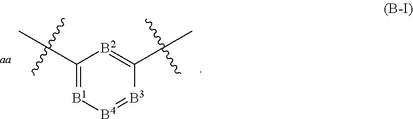

144. The compound of any one of claims 1-143, wherein B⁴ is CR¹.

145. The compound of any one of claims 1-144, wherein B⁴ is CH.

146. The compound of any one of claims 1-145, wherein B¹ is CR¹.

147. The compound of any one of claims 1-146, wherein B¹ is CH.

148. The compound of any one of claims 1-147, wherein B¹ is CR.

149. The compound of any one of claims 1-148, wherein B³ is CH.

150. The compound of any one of claims 1-149, wherein B² is N.

151. The compound of any one of claims 1-143, wherein Ring B is

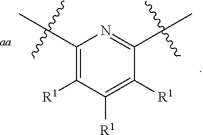

152. The compound of any one of claims 1-143 or 151, wherein Ring B is

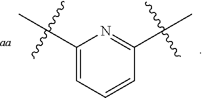 [SMILES: *c1cccc(*)n1]

153. The compound of any one of claims 1-142, wherein Ring B is

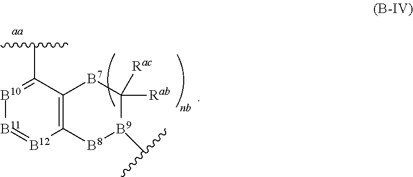

154. The compound of any one of claims 1-142 or 153, wherein Ring B is

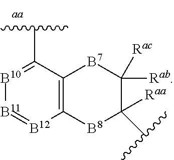

155. The compound of any one of claims 1-142 or 153, wherein Ring B is

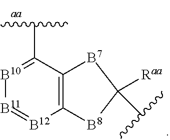

156. The compound of any one of claims 1-142, wherein Ring B is

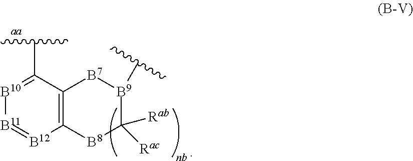

157. The compound of any one of claims 1-142 or 156, wherein Ring B is

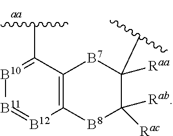

158. The compound of any one of claims 153-157, wherein B⁷ is —O—.

159. The compound of any one of claims 153-158, wherein B⁸ is —O—.

160. The compound of any one of claims 153-159, wherein B⁷ is —O—; and B⁸ is —O—.

161. The compound of any one of claims 153-160, wherein R^{aa} is H.

162. The compound of any one of claims 153-160, wherein R^{aa} is (C₁-C₃)alkyl.

163. The compound of any one of claims 153-160 or 162, wherein R^{aa} is methyl.

164. The compound of any one of claims 153-154 or 156-163, wherein R^{ab} is H.

165. The compound of any one of claims 153-154 or 156-164, wherein R^{ac} is H.

166. The compound of any one of claims 153-154 or 156-160, wherein R^{aa}, R^{ab}, and R^{ac} are each H.

167. The compound of any one of claims 153-154 or 156-160, wherein R^{aa} is (C₁-C₃)alkyl; and R^{ab} and R^{ac} are H.

168. The compound of any one of claims 153-167, wherein B¹⁰ is CR¹.

169. The compound of any one of claims 153-168, wherein B¹⁰ is CH.

170. The compound of any one of claims 153-169, wherein B¹¹ is CR¹.

171. The compound of any one of claims 153-170, wherein B¹¹ is CH.

172. The compound of any one of claims 153-171, wherein B¹² is CR¹.

173. The compound of any one of claims 153-172, wherein B¹² is CH.

174. The compound of any one of claims 1-142 or 153, wherein Ring B is 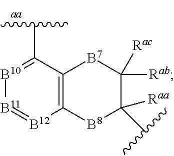
B⁷ and B⁸ are —O—; and R^{aa} is H or (C₁-C₃)alkyl.

175. The compound of any one of claims 1-142 or 153, wherein Ring B is 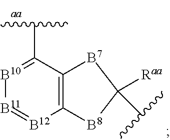
B⁷ and B⁸ are —O—; and R^{aa} is H or (C₁-C₃)alkyl.

176. The compound of any one of claims 1-142 or 156, wherein Ring B is 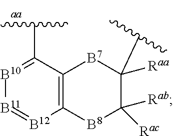
B⁷ and B⁸ are —O—; and R^{aa} is H or (C₁-C₃)alkyl.

177. The compound of any one of claims 174-176, wherein R^{aa} is H.

178. The compound of any one of claims 174-176, wherein R^{aa} is (C₁-C₃)alkyl.

179. The compound of any one of claims 174 or 176-178, wherein R^{ab} and R^{ac} are H.

180. The compound of any one of claims 174-179, wherein B¹⁰, B¹¹, and B¹² are each independently selected CR¹.

181. The compound of any one of claims 174-180, wherein B¹⁰, B¹¹, and B¹² are CH.

182. The compound of any one of claims 154-155 or 157-181, wherein the carbon atom to which L⁴ and R^{aa} are both attached has (R)-configuration.

183. The compound of any one of claims 154-155 or 157-181, wherein the carbon atom to which L⁴ and R^{aa} are both attached has (S)-configuration.

184. The compound of any one of claims 1-142, wherein Ring B is 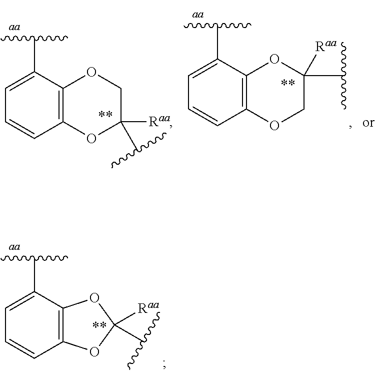
and the carbon atom labelled with ** has (R)-configuration; or wherein Ring B is 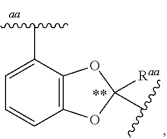
and the carbon atom labelled with ** has (S)-configuration.

185. The compound of any one of claims 1-184, wherein L⁴ is a bond.

186. The compound of any one of claims 153-184, wherein L⁴ is a bond.

187. The compound of any one of claims 1-184, wherein L⁴ is —Z¹—Z²—*, wherein * represents the point of attachment to Ring C.

188. The compound of any one of claims 1-184 or 187, wherein Z¹ is —O—.

189. The compound of any one of claims 1-184 or 187, wherein Z¹ is a bond.

190. The compound of any one of claims 1-184 or 187-189, wherein Z² is —CH₂— optionally substituted with 1-2 R^{c}.

191. The compound of any one of claims 1-184 or 187-190, wherein Z² is —CH₂—.

192. The compound of any one of claims 1-184, wherein L⁴ is —O—CH₂—*, wherein * represents the point of attachment to Ring C.

193. The compound of any one of claims 1-184, wherein L⁴ is —CH₂—.

194. The compound of any one of claims 1-141 or 143-184, wherein L⁴ is a bond.

195. The compound of any one of claims 1-141 or 143-184, wherein L⁴ is —O—Z²—*, wherein * represents the point of attachment to Ring C; and Z² is —CH₂-optionally substituted with 1-2 R^{c}, such as wherein Z² is —CH₂—.

196. The compound of any one of claims 1-140 or 142-184, wherein L³ is —O—; and L⁴ is —CH₂—.

197. The compound of any one of claims 1-196, wherein Ring C is selected from the group consisting of: phenyl, 5- to 6-membered heteroaryl, and 5- to 10-membered bicycloheteroaryl.

198. The compound of any one of claims 1-197, wherein Ring C is selected from the group consisting of: phenyl and 6-membered heteroaryl.

199. The compound of any one of claims 1-198, wherein Ring C is phenyl.

200. The compound of any one of claims 1-199, wherein b is 1-3.

201. The compound of any one of claims 1-200, wherein b is 2.

202. The compound of any one of claims 1-200, wherein b is 1.

203. The compound of any one of claims 1-199, wherein b is 0.

204. The compound of any one of claims 1-199, wherein Ring C is phenyl; and b is 2.

205. The compound of any one of claims 1-199 or 204, wherein 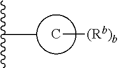
is

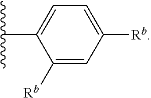

206. The compound of any one of claims 1-199, wherein Ring C is phenyl; and b is 1.

207. The compound of any one of claims 1-199 or 206, wherein 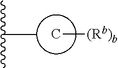
is

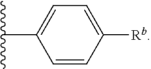

208. The compound of any one of claims 1-199, wherein Ring C is phenyl; and b is 0.

209. The compound of any one of claims 1-207, wherein each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN.

210. The compound of any one of claims 1-207 or 209, wherein each occurrence of R^{b} is independently selected from the group consisting of —F, —Cl, CF₃, and CN.

211. The compound of claim 1, wherein the compound is a compound of Formula (I-A2) or a pharmaceutically acceptable salt thereof: 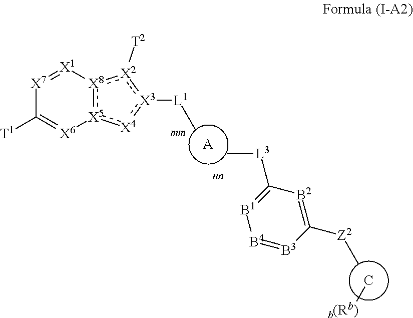
wherein L³ is selected from the group consisting of: —O—; —S—; —C(R^{a}R^{a})—; —N(H)—; —N(C₁₋₃ alkyl)-; —C(═O)—; and —S(O)₁₋₂—; and
Z² is selected from the group consisting of: NH, N(C₁-C₆ alkyl), O, C(═O), S(O)₀₋₂, and —CR^{cA}R^{cB}—,
wherein R^{cA} and R^{cB} are independently selected from the group consisting of H and R^{c}.

212. The compound of claim 211, wherein L³ is —O—.

213. The compound of claims 212 or 216, wherein Z² is —CR^{cA}R^{cB}—.

214. The compound of any one of claims 211-213, wherein Z² is —CH₂—.

215. The compound of any one of claims 211-214, wherein the ring containing B¹, B², B³, and B⁴ is

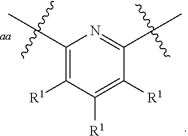

216. The compound of any one of claims 211-215, wherein the ring containing B¹, B², B³, and B⁴ is

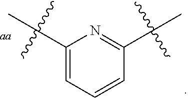 [SMILES: *c1cccc(*)n1]

217. The compound of any one of claims 211-216, wherein X³ is C; X² and X⁴ are N; and X⁵ and X⁸ are C.

218. The compound of any one of claims 211-217, wherein the 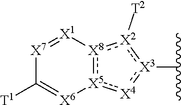
moiety is

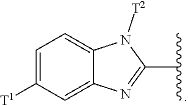

219. The compound of any one of claims 211-217, wherein the 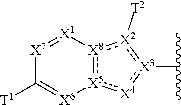
moiety is

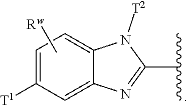

220. The compound of any one of claims 211-217, wherein the 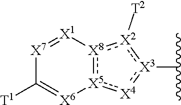
moiety is

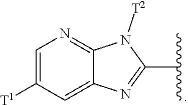

221. The compound of any one of claims 211-217, wherein the 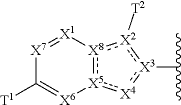
moiety is

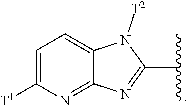

222. The compound of any one of claims 211-217, wherein the 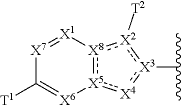
moiety is

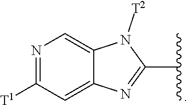

223. The compound of any one of claims 211-222, wherein T¹ is —NR^{s}C(═O)R^{z} or —NR^{s}S(O)₁₋₂—R^{z}; and R^{z} is (C₁-C₆)alkyl optionally substituted with 1-4 R^{v}.

224. The compound of claim 223, wherein R^{z} is (C₁-C₃)alkyl.

225. The compound of claim 223, wherein R^{z} is (C₁-C₃)alkyl substituted with (C₁-C₃)alkoxy; or wherein R^{z} is (C₁-C₃)alkyl substituted with 1-3 independently selected halo, such as wherein R^{z} is —CH₂CF₃.

226. The compound of any one of claims 211-222, wherein T¹ is —NR^{s}C(═O)R^{z} or —NR^{s}S(O)₁₋₂—R^{z}; and R^{z} is —R^{z3} or —CH₂—R^{z3}.

227. The compound of claim 226, wherein R^{z3} is selected from the group consisting of: phenyl, (C₃-C₆)cycloalkyl, and 3- to 6-membered heterocycloalkyl, each optionally substituted with 1-2 R^{v}, such as wherein R^{z3} is phenyl, cyclopropyl, cyclobutyl, difluorocyclobutyl, cycopentyl, oxetanyl, tetrahydropyranyl, or tetrahydro-2H-thiopyran 1,1-dioxide.

228. The compound of any one of claims 223-227, wherein T¹ is —NHC(═O)R^{z}.

229. The compound of any one of claims 223-227, wherein T¹ is —NHS(O)₂—R^{z}.

230. The compound of any one of claims 211-222, wherein T¹ is —(CR^{x}R^{x})_{q}—S(O)₂R^{z}; and q is 1, 2, or 3.

231. The compound of claim 230, wherein R^{z} is (C₁-C₆)alkyl optionally substituted with 1-4 R^{v}.

232. The compound of claims 230 or 231, wherein R^{z} is (C₁-C₃)alkyl.

233. The compound of any one of claims 230-232, wherein q is 1.

234. The compound of any one of claims 230-233, wherein each R^{x} is hydrogen.

235. The compound of any one of claims 211-222, wherein T¹ is —(CR^{x}R^{x})_{q}—C(═O)OH; and q is 1, 2, or 3.

236. The compound of claim 235, wherein q is 1.

237. The compound of claim 235, wherein q is 2.

238. The compound of any one of claims 235-237, wherein each R^{x} is hydrogen.

239. The compound of any one of claims 235-237, wherein one pair of R^{x} on the same carbon, taken together with the carbon atom to which each is attached, forms a (C₃-C₈)cycloalkyl ring; and each remaining R^{x} when present is hydrogen.

240. The compound of claim 239, wherein the pair of R^{x} on the same carbon, taken together with the carbon atom to which each is attached, forms a cyclopropyl ring.

241. The compound of any one of claims 211-222 or 235, wherein T¹ is

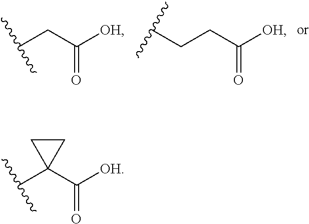

242. The compound of any one of claims 211-222, wherein T¹ is —O—(CR^{x}R^{x})_{q}—C(═O)OH; and q is 1, 2, or 3.

243. The compound of claim 242, wherein q is 1.

244. The compound of claims 242 or 243, wherein each R^{x} is hydrogen.

245. The compound of any one of claims 211-222, wherein T¹ is —P(═O)R^{z1}R^{z2}.

246. The compound of claim 245, wherein R^{z1} and R^{z2} are independently selected (C₁-C₃)alkyl.

247. The compound of any one of claims 211-222, wherein T¹ is 5-membered heteroaryl having 2-4 ring heteroatoms selected from the group consisting of N, O, and S, wherein the heteroaryl is optionally substituted with 1-2 R^{v}.

248. The compound of any one of claims 211-222 or 247, wherein T¹ is selected from the group consisting of:

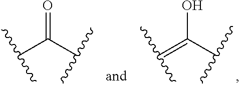

249. The compound of any one of claims 211-222, wherein T¹ is 5- to 6-membered heteroaryl that comprises an endocyclic group selected from the group consisting of: 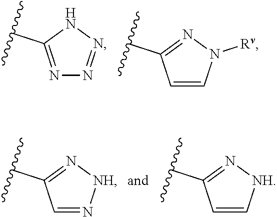
wherein the heteroaryl is further optionally substituted with 1-4 R^{v}.

250. The compound of any one of claims 211-222 or 249, wherein T¹ is selected from the group consisting of:

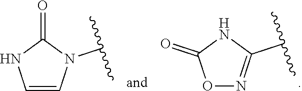 [SMILES: *n1cc[nH]c1=O ; *c1noc(=O)[nH]1]

251. The compound of any one of claims 211-222, wherein T¹ is 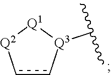
Q¹ is C(═O) or S(O)₂; Q² is O, NH, —CH₂—, or —CH₂—CH₂—; Q³ is N or CH; and [symbol] is a single bond or a double bond, provided that T³ is non-aromatic.

252. The compound of any one of claims 211-222 or 251, wherein T¹ is selected from the group consisting of:

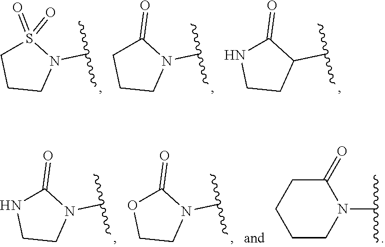

253. The compound of any one of claims 211-252, wherein T² is (C₁-C₆)alkyl which is substituted with (C₁-C₆)alkoxy, (C₁-C₆)thioalkoxy, (C₁-C₆)haloalkoxy, S(O)₂(C₁-C₆ alkyl), (C₃-C₆)cycloalkyl, 3- to 6-membered heterocycloalkyl, phenyl, or 5- to 6-membered heteroaryl, wherein each of the (C₃-C₆)cycloalkyl, 3- to 6-membered heterocycloalkyl, phenyl, or 5- to 6-membered heteroaryl is optionally substituted with 1-4 R^{T}.

254. The compound of any one of claims 211-253, wherein T² is (C₁-C₃)alkyl which is substituted with 4- to 6-membered heterocycloalkyl.

255. The compound of any one of claims 211-254, wherein T² is (C₁-C₃)alkyl which is substituted with oxetanyl.

256. The compound of any one of claims 211-255, wherein T² is 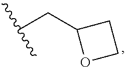 [SMILES: *CC1CCO1]
optionally wherein the stereogenic center in T² has (S)-configuration.

257. The compound of any one of claims 211-256, wherein L¹ is CH₂; and Ring A is

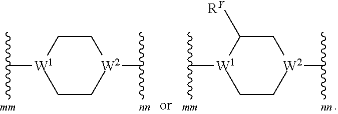

258. The compound of any one of claims 211-257, wherein Ring A is

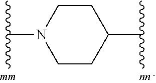 [SMILES: *C1CCN(*)CC1]

259. The compound of any one of claims 211-256, wherein L¹ is a bond; and Ring A is

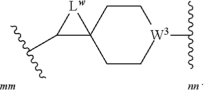

260. The compound of any one of claims 211-256 or 259, wherein Ring A is

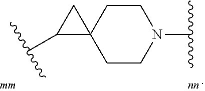 [SMILES: *C1CC12CCN(*)CC2]

261. The compound of any one of claims 211-256, wherein L¹ is CH₂; and Ring A is 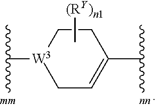
W³ is N or CH; and n1 is 0, 1, or 2.

262. The compound of any one of claims 211-256 or 261, wherein Ring A is

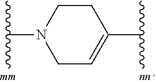 [SMILES: *C1=CCN(*)CC1]

263. The compound of any one of claims 211-256 or 262, wherein Ring A is

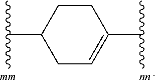 [SMILES: *C1=CCC(*)CC1]

264. The compound of any one of claims 211-263, wherein Ring C is selected from the group consisting of: phenyl and 6-membered heteroaryl; and b is 1 or 2.

265. The compound of any one of claims 211-264, wherein 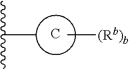
is

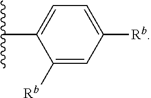

266. The compound of any one of claims 211-264, wherein 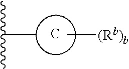
is

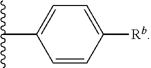

267. The compound of any one of claims 211-264, wherein 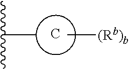
is

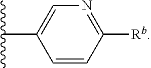

268. The compound of any one of claims 211-267, wherein each occurrence of R^{b} is independently selected from the group consisting of: (C₁-C₆)alkyl, (C₁-C₆)haloalkyl, (C₁-C₆)alkoxy, (C₁-C₆)haloalkoxy, halogen, and CN.

269. The compound of any one of claims 211-268, wherein each occurrence of R^{b} is independently selected from the group consisting of —F, —Cl, CF₃, and CN.

270. The compound of any one of claims 1-269, wherein the compound of Formula I is selected from the group consisting of the compounds in Table C1 or Table C2, or a pharmaceutically acceptable salt or solvate thereof.

271. A pharmaceutical composition comprising a compound of any one of claims 1-270, or a pharmaceutically acceptable salt or solvate thereof, and a pharmaceutically acceptable excipient.

272. A method of treating type 2 diabetes mellitus in a patient in need thereof, the method comprising administering to the patient a therapeutically effective amount of a compound of any one of claims 1-270, or a pharmaceutically acceptable salt or solvate thereof, or a pharmaceutical composition according to claim 271.

273. A method for treating type 2 diabetes mellitus in a patient, the method comprising administering to a patient identified or diagnosed as having type 2 diabetes mellitus a therapeutically effective amount of a compound of any one of claims 1-270, or a pharmaceutically acceptable salt or solvate thereof, or a pharmaceutical composition according to claim 271.

274. A method of treating diabetes mellitus in a patient, the method comprising:
a) determining that the patient has type 2 diabetes mellitus; and
b) administering to the patient a therapeutically effective amount of a compound of any one of claims 1-270, or a pharmaceutically acceptable salt or solvate thereof, or a pharmaceutical composition according to claim 271.

275. The method of any one of claims 272-274, wherein the step of determining that the patient has type 2 diabetes mellitus includes performing an assay to determine the level of an analyte in a sample from the patient, wherein the analyte is selected from the group consisting of hemoglobin A1c (HbA1c), fasting plasma glucose, non-fasting plasma glucose, or any combination thereof.

276. The method of claim 275, wherein the level of HbA1c is greater than or about 6.5%.

277. The method of claims 275 or 276, wherein the level of fasting plasma glucose is greater than or about 126 mg/dL.

278. The method of claims 275 or 276, wherein the level of non-fasting plasma glucose is greater than or about 200 mg/dL.

279. The method of any one of claims 272-278, further comprising obtaining a sample from the patient.

280. The method of claim 279, wherein the sample is a body fluid sample.

281. The method of any one of claims 272-280, wherein the patient is about 40 to about 70 years old and is overweight or obese.

282. The method of any one of claims 272-281, wherein the patient has a body mass index (BMI) greater than or about 22 kg/m².

283. The method of any one of claims 272-282, wherein the patient has a BMI greater than or about 30 kg/m².

284. The method of any one of claims 272-283, wherein the treatment of type 2 diabetes mellitus comprises a reduction in fasting plasma glucose levels.

285. The method of claim 284, wherein the fasting plasma glucose levels are reduced to about or below 100 mg/dL.

286. The method of any one of claims 272-285, wherein the treatment of type 2 diabetes mellitus comprises a reduction in HbA1c levels.

287. The method of claim 286, wherein the HbA1c levels are reduced to about or below 5.7%.

288. The method of any one of claims 272-287, wherein the treatment of type 2 diabetes mellitus comprises a reduction in glucagon levels.

289. The method of any one of claims 272-288, wherein the treatment of type 2 diabetes mellitus comprises an increase in insulin levels.

290. The method of any one of claims 272-289, wherein the treatment of type 2 diabetes mellitus comprises a decrease in BMI.

291. The method of claim 290, wherein the BMI is decreased to about or below 25 kg/m².

292. The method of any of one of claims 272-291, wherein the compound of any one of claims 1-270, or a pharmaceutically acceptable salt or solvate thereof, or a pharmaceutical composition according to claim 271, is administered orally.

293. The method of any one of claims 272-292, further comprising administering an additional therapy or therapeutic agent to the patient.

294. The method of claim 293, wherein the additional therapy or therapeutic agent is selected from the group consisting of an antidiabetic agent, an anti-obesity agent, a GLP-1 receptor agonist, an anti-emetic agent, an agent to treat non-alcoholic steatohepatitis (NASH), gastric electrical stimulation, dietary monitoring, physical activity, or any combinations thereof.

295. The method of claim 294, wherein the antidiabetic agent is selected from the group consisting of a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor, or any combinations thereof.

296. The method of claim 295, wherein the biguanide is metformin.

297. The method of claim 294, wherein the anti-obesity agent is selected from the group consisting of neuropeptide Y receptor type 2 (NPYR2) agonist, a NPYR1 or NPYR5 antagonist, a human proislet peptide (HIP), a cannabinoid receptor type 1 (CB1R) antagonist, a lipase inhibitor, a melanocortin receptor 4 agonist, a farnesoid X receptor (FXR) agonist, phentermine, zonisamide, a norepinephrine/dopamine reuptake inhibitor, a GDF-15 analog, an opioid receptor antagonist, a cholecystokinin agonist, a serotonergic agent, a methionine aminopeptidase 2 (MetAP2) inhibitor, diethylpropion, phendimetrazine, benzphetamine, a fibroblast growth factor receptor (FGFR) modulator, an AMP-activated protein kinase (AMPK) activator, or any combinations thereof.

298. The method of claim 294, wherein the GLP-1 receptor agonist is selected from the group consisting of liraglutide, exenatide, dulaglutide, albiglutide, taspoglutide, lixisenatide, semaglutide, or any combinations thereof.

299. The method of claim 294, wherein the agent to treat NASH is selected from the group consisting of an FXR agonist, PF-05221304, a synthetic fatty acid-bile conjugate, an anti-lysyl oxidase homologue 2 (LOXL2) monoclonal antibody, a caspase inhibitor, a MAPK5 inhibitor, a galectin 3 inhibitor, a fibroblast growth factor 21 (FGF21) agonist, a niacin analogue, a leukotriene D4 (LTD4) receptor antagonist, an acetyl-CoA carboxylase (ACC) inhibitor, a ketohexokinase (KHK) inhibitor, an ileal bile acid transporter (IBAT) inhibitor, an apoptosis signal-regulating kinase 1 (ASK1) inhibitor, or any combinations thereof.

300. The method of any one of claims 293-299, wherein the compound of any one of claims 1-270 or a pharmaceutically acceptable salt or solvate thereof, or a pharmaceutical composition according to claim 271, and the additional therapeutic agent are administered as separate dosages sequentially in any order.

301. A method for modulating insulin levels in a patient in need of such modulating, the method comprising administering to the patient an effective amount of a compound as claimed in any one of claims 1-270, or a pharmaceutically acceptable salt or solvate thereof, or a pharmaceutical composition according to claim 271.

302. The method of claim 301, wherein the modulation results in an increase of insulin levels.

303. A method for modulating glucose levels in a patient in need of such modulating, the method comprising administering to the patient an effective amount of a compound as claimed in any one of claims 1-270, or a pharmaceutically acceptable salt or solvate thereof, or a pharmaceutical composition according to claim 271.

304. The method of claim 303, wherein the modulation results in a decrease of glucose levels.

305. A method for treating a GLP-1 associated disease, disorder, or condition, the method comprising administering to a patient in need thereof an effective amount of a compound as claimed in any one of claims 1-270, or a pharmaceutically acceptable salt or solvate thereof, or a pharmaceutical composition according to claim 271.

306. The method of claim 305, wherein the disease, disorder, or condition is selected from the group consisting of type 1 diabetes mellitus, type 2 diabetes mellitus, early onset type 2 diabetes mellitus, idiopathic type 1 diabetes mellitus (Type 1b), youth-onset atypical diabetes (YOAD), maturity onset diabetes of the young (MODY), latent autoimmune diabetes in adults (LADA), obesity, weight gain from use of other agents, gout, excessive sugar craving, hypertriglyceridemia, dyslipidemia, malnutrition-related diabetes, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, traumatic brain injury, peripheral vascular disease, endothelial dysfunction, impaired vascular compliance, vascular restenosis, thrombosis, hypertension, pulmonary hypertension, restenosis after angioplasty, intermittent claudication, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, macular degeneration, cataract, glomerulosclerosis, arthritis, osteoporosis, treatment of addiction, cocaine dependence, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), ulcerative colitis, inflammatory bowel disease, colitis, irritable bowel syndrome, Crohn's disease, short bowel syndrome, Parkinson's, Alzheimer's disease, impaired cognition, schizophrenia, Polycystic Ovary Syndrome (PCOS), or any combination thereof.

307. The method of claim 306, wherein the disease, disorder, or condition is selected from the group consisting of type 2 diabetes mellitus, early onset type 2 diabetes mellitus, obesity, weight gain from use of other agents, gout, excessive sugar craving, hypertriglyceridemia, dyslipidemia, gestational diabetes, kidney disease, adipocyte dysfunction, sleep apnea, visceral adipose deposition, eating disorders, cardiovascular disease, congestive heart failure, myocardial infarction, left ventricular hypertrophy, peripheral arterial disease, stroke, hemorrhagic stroke, ischemic stroke, transient ischemic attacks, atherosclerotic cardiovascular disease, hyperglycemia, post-prandial lipemia, metabolic acidosis, ketosis, hyperinsulinemia, impaired glucose metabolism, insulin resistance, hepatic insulin resistance, alcohol use disorder, chronic renal failure, metabolic syndrome, syndrome X, smoking cessation, premenstrual syndrome, angina pectoris, diabetic nephropathy, impaired glucose tolerance, diabetic neuropathy, diabetic retinopathy, bipolar disorder/major depressive disorder, skin and connective tissue disorders, foot ulcerations, psoriasis, primary polydipsia, non-alcoholic steatohepatitis (NASH), non-alcoholic fatty liver disease (NAFLD), short bowel syndrome, Parkinson's disease, Polycystic Ovary Syndrome (PCOS), or any combination thereof.

308. The method of claim 307, wherein the disease, disorder, or condition includes, but is not limited to type 2 diabetes mellitus, early onset type 2 diabetes mellitus, obesity, weight gain from use of other agents, gout, excessive sugar craving, hypertriglyceridemia, dyslipidemia, gestational diabetes, adipocyte dysfunction, visceral adipose deposition, myocardial infarction, peripheral arterial disease, stroke, transient ischemic attacks, hyperglycemia, post-prandial lipemia, metabolic acidosis, ketosis, hyperinsulinemia, impaired glucose metabolism, insulin resistance, hepatic insulin resistance, chronic renal failure, syndrome X, angina pectoris, diabetic nephropathy, impaired glucose tolerance, diabetic neuropathy, diabetic retinopathy, skin and connective tissue disorders, foot ulcerations, or any combination thereof.
